(19) United States
(12) Patent Application Publication
(10) Pub. No.: US 2023/0202989 A1
(43) Pub. Date: Jun. 29, 2023

(54) COMPOUNDS

(71) Applicant: JANSSEN PHARMACEUTICA NV, Beerse (BE)

(72) Inventors: Michael Eric MURATORE, Beerse (BE); Nina VAN OPDENBOSCH, Beerse (BE); Joseph Elisabeth LEENAERTS, Beerse (BE); Mohamed LAMKANFI, Beerse (BE); Gary John TRESADERN, Beerse (BE); Daniel OEHLRICH, Beerse (BE); Michiel Luc Maria VAN GOOL, Madrid (ES); Laura PEREZ BENITO, Beerse (BE)

(21) Appl. No.: 17/999,627

(22) PCT Filed: May 27, 2021
(86) PCT No.: PCT/EP2021/064221

§ 371 Date: Nov. 22, 2022

(30) Foreign Application Priority Data
May 28, 2020 (EP) 20382456.0

(51) Int. Cl. C07D 237/32; C07D 471/04; C07D 403/12; C07D 487/04; C07D 405/14

(52) U.S. Cl. CPC C07D 237/32; C07D 471/04; C07D 403/12; C07D 487/04; C07D 405/14

(57) ABSTRACT

The invention relates to novel compounds for use as inhibitors of NLRP3 inflammasone production, wherein such compounds are as defined by compounds of formula (I) and wherein the integers R¹, R² and R³ are defined in the description, and where the compounds may be useful as medicaments, for instance for use in the treatment of a disease or disorder that is associated with NLRP3 inflammasome activity.

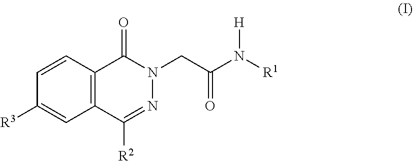

### FIELD OF THE INVENTION

The present invention relates to novel triazinones that are useful as inhibitors of NOD-like receptor protein 3 (NLRP3) inflammasome pathway. The present invention also relates to processes for the preparation of said compounds, pharmaceutical compositions comprising said compounds, methods of using said compounds in the treatment of various diseases and disorders, and medicaments containing them, and their use in diseases and disorders mediated by NLRP3.

### BACKGROUND OF THE INVENTION

Inflammasomes, considered as central signalling hubs of the innate immune system, are multi-protein complexes that are assembled upon activation of a specific set of intracellular pattern recognition receptors (PRRs) by a wide variety of pathogen- or danger-associated molecular patterns (PAMPs or DAMPs). To date, it was shown that inflammasomes can be formed by nucleotide-binding oligomerization domain (NOD)-like receptors (NLRs) and Pyrin- and HIN200-domain-containing proteins (Van Opdenbosch N and Lamkanfi M. Immunity, 2019 Jun. 18; 50(6):1352-1364). The NLRP3 inflammasome is assembled upon detection of environmental crystals, pollutants, host-derived DAMPs and protein aggregates (Tartey S and Kanneganti T D. Immunology, 2019 April; 156(4):329-338). Clinically relevant DAMPs that engage NLRP3 include uric acid and cholesterol crystals that cause gout and atherosclerosis, amyloid-β fibrils that are neurotoxic in Alzheimer's disease and asbestos particles that cause mesothelioma (Kelley et al., Int J Mol Sci, 2019 Jul. 6; 20(13)). Additionally, NLRP3 is activated by infectious agents such as Vibrio cholerae; fungal pathogens such as Aspergillus fumigatus and Candida albicans; adenoviruses, influenza A virus and SARS-CoV-2 (Tartey and Kanneganti, 2019 (see above); Fung et al. Emerg Microbes Infect, 2020 Mar. 14; 9(1):558-570).

Although the precise NLRP3 activation mechanism remains unclear, for human monocytes, it has been suggested that a one-step activation is sufficient while in mice a two-step mechanism is in place. Given the multitude in triggers, the NLRP3 inflammasome requires add-on regulation at both transcriptional and post-transcriptional level (Yang Y et al., Cell Death Dis, 2019 Feb. 12; 10(2):128).

The NLRP3 protein consists of an N-terminal pyrin domain, followed by a nucleotide-binding site domain (NBD) and a leucine-rich repeat (LRR) motif on C-terminal end (Sharif et al., Nature, 2019 June; 570(7761):338-343). Upon recognition of PAMP or DAMP, NLRP3 aggregates with the adaptor protein, apoptosis-associated speck-like protein (ASC), and with the protease caspase-1 to form a functional inflammasome. Upon activation, procaspase-1 undergoes autoproteolysis and consequently cleaves gasdermin D (Gsdmd) to produce the N-terminal Gsdmd molecule that will ultimately lead to pore-formation in the plasma membrane and a lytic form of cell death called pyroptosis. Alternatively, caspase-1 cleaves the pro-inflammatory cytokines pro-IL-1β and pro-IL-18 to allow release of its biological active form by pyroptosis (Kelley et al., 2019—see above).

Dysregulation of the NLRP3 inflammasome or its downstream mediators are associated with numerous pathologies ranging from immune/inflammatory diseases, auto-immune/auto-inflammatory diseases (Cryopyrin-associated Periodic Syndrome (Miyamae T. Paediatr Drugs, 2012 Apr. 1; 14(2):109-17); sickle cell disease; systemic lupus erythematosus (SLE)) to hepatic disorders (eg. non-alcoholic steatohepatitis (NASH), chronic liver disease, viral hepatitis, alcoholic steatohepatitis, and alcoholic liver disease) (Szabo G and Petrasek J. Nat Rev Gastroenterol Hepatol, 2015 July; 12(7):387-400) and inflammatory bowel diseases (eg. Crohn's disease, ulcerative colitis) (Zhen Y and Zhang H. Front Immunol, 2019 Feb. 28; 10:276). Also, inflammatory joint disorders (eg. gout, pseudogout (chondrocalcinosis), arthropathy, osteoarthritis, and rheumatoid arthritis (Vande Walle L et al., Nature, 2014 Aug. 7; 512(7512):69-73) were linked to NLRP3. Additionally, kidney related diseases (hyperoxaluria (Knauf et al., Kidney Int, 2013 November; 84(5):895-901), lupus nephritis, hypertensive nephropathy (Krishnan et al., Br J Pharmacol, 2016 February; 173(4):752-65), hemodialysis related inflammation and diabetic nephropathy which is a kidney-related complication of diabetes (Type 1, Type 2 and mellitus diabetes), also called diabetic kidney disease (Shahzad et al., Kidney Int, 2015 January; 87(1):74-84) are associated to NLRP3 inflammasome activation. Reports link onset and progression of neuroinflammation-related disorders (eg. brain infection, acute injury, multiple sclerosis, Alzheimer's disease) and neurodegenerative diseases (Parkinsons disease) to NLRP3 inflammasome activation (Sarkar et al., NPJ Parkinsons Dis, 2017 Oct. 17; 3:30). In addition, cardiovascular or metabolic disorders (eg. cardiovascular risk reduction (CvRR), atherosclerosis, type I and type II diabetes and related complications (e.g. nephropathy, retinopathy), peripheral artery disease (PAD), acute heart failure and hypertension (Ridker et al., CANTOS Trial Group. N Engl J Med, 2017 Sep. 21; 377(12):1119-1131; and Toldo S and Abbate A. Nat Rev Cardiol, 2018 April; 15(4):203-214) have recently been associated to NLRP3. Also, skin associated diseases were described (eg. wound healing and scar formation; inflammatory skin diseases, eg. acne, hidradenitis suppurativa (Kelly et al., Br J Dermatol, 2015 December; 173(6)). In addition, respiratory conditions have been associated with NLRP3 inflammasome activity (eg. asthma, sarcoidosis, Severe Acute Respiratory Syndrome (SARS) (Nieto-Torres et al., Virology, 2015 November; 485:330-9)) but also age-related macular degeneration (Doyle et al., Nat Med, 2012 May; 18(5):791-8). Several cancer related diseases/disorders were described linked to NLRP3 (eg. myeloproliferative neoplasms, leukemias, myelodysplastic syndromes (MOS), myelofibrosis, lung cancer, colon cancer (Ridker et al., Lancet, 2017 Oct. 21; 390(10105):1833-1842; Derangere et al., Cell Death Differ. 2014 December; 21(12):1914-24; Basiorka et al., Lancet Haematol, 2018 September; 5(9): e393-e402, Zhang et al., Hum Immunol, 2018 January; 79(1):57-62).

Several patent applications describe NLRP3 inhibitors, with recent ones including for instance international patent application WO 2020/018975, WO 2020/037116, WO 2020/021447, WO 2020/010143, WO 2019/079119, WO 2019/0166621 and WO 2019/121691, which disclose a range of specific compounds.

There is a need for inhibitors of the NLRP3 inflammasome pathway to provide new and/or alternative treatments for the diseases/disorders mentioned herein.

### SUMMARY OF THE INVENTION

The invention provides compounds which inhibit the NLRP3 inflammasome pathway.

Thus, in an aspect of the invention, there is now provided a compound of formula (I),

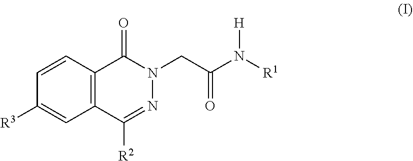

or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) C₁₋₃ alkyl optionally substituted with one or more substituents independently selected from halo, —OH and —OC₁₋₃ alkyl;
(ii) C₃₋₆ cycloalkyl;
(iii) C₂₋₄ alkenyl optionally substituted with —OC₁₋₃ alkyl; or
(iv) —N(R^{2a})R^{2b};
   R^{2a} and R^{2b} each represent hydrogen or C₁₋₄ alkyl, or R^{2a} and R^{2b} may be linked together to form a 3- to 4-membered ring optionally substituted by one or more fluoro atoms;
   R³ represents:
(i) hydrogen;
(ii) halo;
(iii) C₁₋₄ alkyl optionally substituted with one or more substituents independently selected from halo, —OH and —OC₁₋₃ alkyl;
(iv) C₂₋₄ alkenyl optionally substituted with —OC₁₋₃ alkyl;
(v) C₃₋₆ cycloalkyl; or
(vi) —OC₁₋₃ alkyl,
   which compounds may be referred to herein as “compounds of the invention”.

In an embodiment, compounds of the invention that may be mentioned include those in which:
(i) when R³ represents hydrogen, R² represents methyl, then R¹ does not represent 4-methylphenyl;
(ii) when R³ represents hydrogen, R² represents cyclohexyl, then R¹ does not represent 2-indanyl (2,3-dihydro-1H-indene linked at the 2-position), which may be referred to herein as “the provisos”.

For instance, there is provided a compound of formula (I) as hereinbefore defined, or a pharmaceutically acceptable salt thereof, for use as an NLRP3 inhibitor (e.g. in the treatment of a disease or disorder that is associated with NLRP3 inflammasome activity), provided that it is not a compound of the provisos. There is also provided a compound of formula (I) as hereinbefore defined, or a pharmaceutically acceptable salt thereof, for use as an NLRP3 inhibitor in the treatment of a cancer, provided that it is not compound (i) of the provisos. There is also provided a compound of formula (I) as hereinbefore defined, or a pharmaceutically acceptable salt thereof, for use as an NLRP3 inhibitor in the treatment of Alzeheimer's disease, provided that it is not compound (ii) of the provisos

In an aspect of the invention, there is provided a compound of formula (I) as hereinbefore defined, or a pharmaceutically acceptable salt thereof, wherein: R¹ represents:
(i) C₃₋₆ cycloalkyl optionally substituted with one or more substituents independently selected from halo, —OH, —C₁₋₃ alkyl (itself optionally substituted by one or more substituents selected from fluoro and —OH) and —OC₁₋₃alkyl;
(ii) aryl or heteroaryl, each of which is optionally substituted with 1 to 3 substituents independently selected from halo, —CN, —OH, —O—C₁₋₃ alkyl, —C₁₋₆ alkyl (e.g. —C₁₋₃ alkyl), haloC₁₋₃alkyl, hydroxyC₁₋₃ alkyl, C₁₋₃ alkoxyC₁₋₃alkyl, haloC₁₋₃alkoxy, aminoC₁₋₃alkyl (e.g. H₂N—C₁₋₃alkyl or (CH₃)₂N—C₁₋₃ alkyl), C₃₋₆ cycloalkyl or aryl/heteroaryl (wherein such latter groups are themselves optionally substituted by one or more substituents selected from halo, C₁₋₃ alkyl and —OC₁₋₃ alkyl); or
(iii) heterocyclyl, optionally substituted with 1 to 3 substituents independently selected from halo, ═O, —OH, —C₁₋₄ alkyl (itself optionally substituted by one or more substituents selected from fluoro, ═O and —OH), —OC₁₋₃alkyl, C₃₋₆ cycloalkyl and a 3-6 membered heterocyclyl ring;
   R² represents:
(i) C₁₋₆ alkyl (e.g C₁₋₄ alkyl or C₁₋₃ alkyl) optionally substituted with one or more substituents independently selected from halo, ═O, —OH and —OC₁₋₃ alkyl;
(ii) C₃₋₆ cycloalkyl optionally substituted by one or more substituents selected from halo (e.g. fluoro), C₁₋₃ alkyl and —OC₁₋₃ alkyl;
(iii) C₂₋₄ alkenyl optionally substituted with —OC₁₋₃ alkyl; or
(iv) —N(R^{2a})R^{2b};
   R^{2a} and R^{2b} each represent hydrogen or C₁₋₄ alkyl, or R^{2a} and R^{2b} may be linked together to form a 3- to 4-membered ring optionally substituted by one or more fluoro atoms;
   R³ represents:
(i) hydrogen;
(ii) halo or —CN;
(iii) C₁₋₆ alkyl (e.g. C₁₋₄ alkyl) optionally substituted with one or more substituents independently selected from halo, —OH and —OC₁₋₃ alkyl;
(iv) C₂₋₄ alkenyl optionally substituted with —OC₁₋₃ alkyl;
(v) C₃₋₆ cycloalkyl optionally substituted by one or more fluoro atoms;
(vi) —NH₂, —N(H)(C₁₋₃alkyl) or N(C₁₋₃alkyl)₂; or
(vii) —OC₁₋₃ alkyl optionally substituted by one or more fluoro atoms;
   and wherein the R³ containing benzene ring may also be optionally substituted (at the three relevant positions) with one substituent selected from halo (e.g. fluoro), —OH and —CN,
   which compounds may also be referred to herein as “compounds of the invention”.

In an embodiment, there is provided a compound of formula (I), as hereinbefore defined, or a pharmaceutically acceptable salt thereof, wherein R³ does not represent hydrogen.

In another aspect, there is provided compounds of the invention for use as a medicament. In another aspect, there is provided a pharmaceutical composition comprising a therapeutically effective amount of a compound of the invention.

In a further aspect, there is provided compounds of the invention (and/or pharmaceutical compositions comprising such compounds) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor. Specific diseases or disorders may be mentioned herein, and may for instance be selected from inflammasome-related diseases or disorders, immune diseases, inflammatory diseases, auto-immune diseases, or auto-inflammatory diseases.

In another aspect, there is provided a use of compounds of the invention (and/or pharmaceutical compositions comprising such compounds): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In another aspect, there is provided use of compounds of the invention (and/or pharmaceutical compositions comprising such compounds) in the manufacture of a medicament for: the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In another aspect, there is provided a method of treating a disease or disorder in which the NLRP3 signalling contributes to the pathology, and/or symptoms, and/or progression, of said disease/disorder, comprising administering a therapeutically effective amount of a compound of the invention, for instance to a subject (in need thereof). In a further aspect there is provided a method of inhibiting the NLRP3 inflammasome activity in a subject (in need thereof), the method comprising administering to the subject in need thereof a therapeutically effective amount of a compound of the invention.

In further aspect, there is a provided a compound of the invention in combination (including a pharmaceutical combination) with one or more therapeutic agents (for instance as described herein). Such combination may also be provided for use as described herein in respect of compounds of the invention, or, a use of such combination as described herein in respect of compounds of the invention. There may also be provided methods as described herein in respect of compounds of the invention, but wherein the method comprises administering a therapeutically effective amount of such combination.

### DETAILED DESCRIPTION OF THE INVENTION

The invention provides a compound of formula (I),

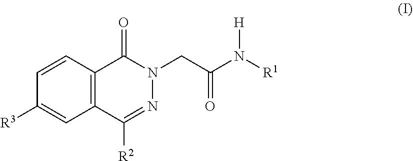

or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
   R² represents:
(i) C₁₋₃ alkyl optionally substituted with one or more substituents independently selected from halo, —OH and —OC₁₋₃ alkyl;
(ii) C₃₋₆ cycloalkyl;
(iii) C₂₋₄ alkenyl optionally substituted with —OC₁₋₃ alkyl; or
(iv) —N(R^{2a})R^{2b};
   R^{2a} and R^{2b} each represent hydrogen or C₁₋₄ alkyl, or R^{2a} and R^{2b} may be linked together to form a 3- to 4-membered ring optionally substituted by one or more fluoro atoms;
   R³ represents:
(i) hydrogen;
(ii) halo;
(iii) C₁₋₄ alkyl optionally substituted with one or more substituents independently selected from halo, —OH and —OC₁₋₃ alkyl;
(iv) C₂₋₄ alkenyl optionally substituted with —OC₁₋₃ alkyl;
(v) C₃₋₆ cycloalkyl; or
(vi) —OC₁₋₃ alkyl.

As indicated above, such compounds may be referred to herein as “compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

When an absolute configuration is specified, it is according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (3H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain. Such a group is attached to the rest of the molecule by a single bond.

C_{2-q} alkenyl when used herein (again where q is the upper limit of the range) refers to an alkyl group that contains unsaturation, i.e. at least one double bond.

C_{3-q} cycloalkyl (where q is the upper limit of the range) refers to an alkyl group that is cyclic, for instance cycloalkyl groups may be monocyclic or, if there are sufficient atoms, bicyclic. In an embodiment, such cycloalkyl groups are monocyclic. Such cycloalkyl groups are unsaturated. Substituents may be attached at any point on the cycloalkyl group.

The term “halo”, when used herein, preferably includes fluoro, chloro, bromo and iodo.

C_{1-q} alkoxy groups (where q is the upper limit of the range) refers to the radical of formula —OR^{a}, where R^{a} is a C_{1-q} alkyl group as defined herein. HaloC_{1-q} alkyl (where q is the upper limit of the range) groups refer to C_{1-q} alkyl groups, as defined herein, where such group is substituted by one or more halo. HydroxyC_{1-q} alkyl (where q is the upper limit of the range) refers to C_{1-q} alkyl groups, as defined herein, where such group is substituted by one or more (e.g. one) hydroxy (—OH) groups (or one or more, e.g. one, of the hydrogen atoms is replaced with —OH). Similarly, haloC_{1-q} alkoxy and hydroxyC_{1-q} alkoxy represent corresponding —OC_{1-q} alkyl groups that are substituted by one or more halo, or, substituted by one or more (e.g. one) hydroxy, respectively.

Heterocyclyl groups that may be mentioned include non-aromatic monocyclic and bicyclic heterocyclyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocyclyl groups may also be bridged. Such heterocyclyl groups are saturated. C_{2-q} heterocyclyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]-heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocyclyl groups may, where appropriate, be located on any atom in the ring system including a heteroatom. The point of attachment of heterocyclyl groups may be via any atom in the ring system including (where appropriate) a heteroatom (such as a nitrogen atom), or an atom on any fused carbocyclic ring that may be present as part of the ring system. Heterocyclyl groups may also be in the N- or S-oxidised form. In an embodiment, heterocyclyl groups mentioned herein are monocyclic.

Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydronaphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. When aryl groups are polycyclic, in an embodiment, each ring is aromatic. In an embodiment, aryl groups mentioned herein are monocyclic or bicyclic. In a further embodiment, aryl groups mentioned herein are monocyclic.

“Heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. In an embodiment, when heteroaryl groups are polycyclic, then each ring is aromatic. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. When heteroaryl groups are polycyclic in which there is a non-aromatic ring present, then that non-aromatic ring may be substituted by one or more ═O group. In an embodiment, heteroaryl groups mentioned herein may be monocyclic or bicyclic. In a further embodiment, heteroaryl groups mentioned herein are monocyclic.

Heteroatoms that may be mentioned include phosphorus, silicon, boron and, preferably, oxygen, nitrogen and sulfur.

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

Various embodiments of the invention will now be described, including embodiments of the compounds of the invention.

In an embodiment, there is provided a compound of formula (I), as hereinbefore defined, or a pharmaceutically acceptable salt thereof, wherein R³ does not represent hydrogen.

In an embodiment, there is provided a compound of formula (I), as hereinbefore defined, or a pharmaceutically acceptable salt thereof, wherein R³ represents:
(i) halo;
(ii) C₁₋₄ alkyl optionally substituted with one or more substituents independently selected from halo, —OH and —OC₁₋₃ alkyl;
(iii) C₂₋₄ alkenyl optionally substituted with —OC₁₋₃ alkyl;
(iv) C₃₋₆ cycloalkyl; or
(v) —OC₁₋₃ alkyl.

In an embodiment, compounds of the invention include those in which R¹ represents: (i) C₃₋₆ cycloalkyl; (ii) aryl or heteroaryl; or (iii) or heterocyclyl, all of which are optionally substituted as herein defined. In a particular embodiment, R¹ represents: (i) C₃₋₆ cycloalkyl; or (ii) aryl or heteroaryl, all of which are optionally substituted as herein defined.

In an embodiment when R¹ represents optionally substituted C₃₋₆ cycloalkyl, then it represents C₃₋₆ cycloalkyl (or, in an embodiment, C₃₋₄ cycloalkyl) optionally substituted by one or two substituents selected from C₁₋₃ alkyl (e.g. methyl) and —OH. In a further embodiment, R¹ represents cyclopropyl (e.g. unsubstituted) or cyclobutyl. In a further embodiment, R¹ represents cyclohexyl. In yet a further embodiment, R¹ represents unsubstituted cyclopropyl or cyclobutyl substituted by —OH and methyl (e.g. at the same carbon atom). In yet a further embodiment, R¹ represents cyclohexyl, for instance substituted by —OH (e.g. by one —OH group). In an embodiment therefore, R¹ represents:

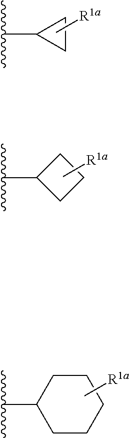

where each R^{1a} represents one or two optional substituents selected from —OH and C₁₋₃ alkyl (e.g. methyl). In a particular embodiment of this aspect, R¹ represents C₃₋₆ cyclolkyl, such as optionally substituted cyclohexyl, optionally substituted cyclobutyl or unsubstituted (or optionally substituted) cyclopropyl, for instance:

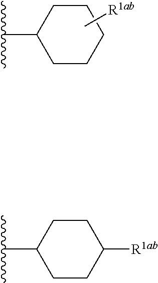

where each R^{1ab} represents one or two optional substituents selected from those defined by R^{1a}, and in an embodiment, represents one optional substituent selected from —OH;

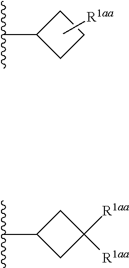

where each R^{1aa} represents one or two optional substituents selected from those defined by R^{1a}, and in an embodiment represents two substituents, methyl and —OH; or

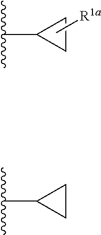

where R^{1a} is as defined above, but where, in a particular embodiment, it is not present.

In an embodiment where R¹ represents aryl or heteroaryl, optionally substituted as defined herein, then it may represent: (i) phenyl; (ii) a 5- or 6-membered mono-cyclic heteroaryl group; or (iii) a 9- or 10-membered bicyclic heteroaryl group, all of which are optionally substituted by one to three substituents as defined herein. In an embodiment, the aforementioned aryl and heteroaryl groups are optionally substituted with one or two (e.g. one) substituent(s) selected from halo (e.g. fluoro), —OH, C₁₋₃ alkyl and —OC₁₋₃ alkyl. In one embodiment, R¹ represents phenyl or a mono-cyclic 6-membered heteroaryl group and in another embodiment it may represent a 9- or 10-membered (e.g. 9-membered) bicyclic heteroaryl group. Hence, in an embodiment, R¹ may represent:

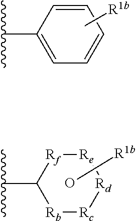

wherein R^{1b} represents one or two optional substituents selected from halo, —CH₃, —OH and —OCH₃ (and in a further embodiment, such optional substituents are selected from fluoro and methoxy), and at least one of R_{b}, Rₑ, R_{d}, Rₑ and R_{f} represents a nitrogen heteroatom (and the others represent CH). In an embodiment, either one or two of R_{b}, Rₑ, R_{d}, Rₑ and R_{f} represent(s) a nitrogen heteroatom, for instance, R_{d} represents nitrogen and, optionally, R_{b} represents nitrogen, or, Rₑ represents nitrogen. In an aspect: (i) R_{b} and R_{d} represent nitrogen; (ii) R_{d} represents nitrogen; or (iii) Rₑ represents nitrogen. Hence, R¹ may represent 3-pyridyl or 4-pyrimidinyl, both of which are optionally substituted as herein defined; however, in an embodiment, such groups are unsubstituted.

In another embodiment, R¹ may represent:

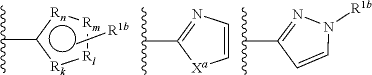

wherein R^{1b} is as defined above (i.e. represents one or two optional substituents) but in an aspect, is preferably not present (and, as such, in an embodiment, represents an unsubstituted 5-membered heteroaryl group), and at least one of Rₖ, Rₗ, Rₘ and Rₙ represents a heteroatom, and in an embodiment, at least one of these represents N and the others are independently selected from CH, N, O and S (provided that the rules of valency are adhered to); for instance, in an embodiment, one of Rₖ and Rₙ represents N, the other represents N, O, S or CH, and Rₗ and Rₘ each represent CH, and, in a further particular embodiment, X^{a} represents N, O, S or CH, for instance X^{a} represents O, so forming a 2-oxazolyl group. As such, in a particular embodiment, R¹ represents unsubstituted 2-oxazolyl. In another particular embodiment, R¹ represents a 3-pyrazolyl group (for instance in which Rₖ and Rₗ represents N, Rₙ and Rₘ represent CH, and R^{1b} represents a C₁₋₄ alkyl (e.g. isopropyl) that is on the 1-(N) atom).

In another embodiment, R¹ may represent:

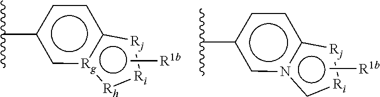

wherein R^{1b} is as defined above (i.e. represents one or two optional substituents as defined above), each ring of the bicyclic system is aromatic, R_{g} represents a N or C atom and any one or two of Rₕ, Rᵢ and Rⱼ (for instance, one or two of Rᵢ and Rⱼ) represents N and the other(s) represent(s) C (provided that, as the skilled person would understand, the rules of valency are adhered to; for instance when one of the atoms of the (hetero)aromatic ring represents C, then it is understood that it may bear a H atom).

In an embodiment R¹ represents:

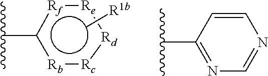

in which R_{b} and R_{d} represent a nitrogen atom, and, in an embodiment, there is no R^{1b} substituent present.

In another embodiment, R¹ represents:

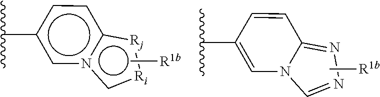

in which one of Rᵢ and Rⱼ represents N and the other represents C, or, both Rᵢ and Rⱼ represent N, and, in an embodiment, there is no R^{1b} substituent present.

In a further embodiment, R¹ represents phenyl or a 6-membered heteroaryl group (containing between one and three heteroatoms) and which is optionally substituted as defined herein. In an embodiment, R¹ represents a 6,5-fused bicyclic ring containing one to five heteroatoms (wherein at least two are nitrogen) and which group is optionally substituted as herein defined.

In a further embodiment, R¹ represents:

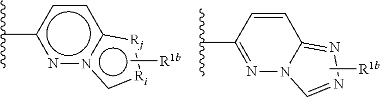

in which Rⁱ, R^{j} and R^{1b} are as hereinbefore defined.

In an embodiment where R¹ represents heterocyclyl, optionally substituted as defined herein, such group is in a further aspect a 5- or 6-membered heterocyclyl group, for instance containing at least one nitrogen or oxygen heteroatom; for instance, in a particular embodiment, in this instance R¹ may represent a 6-membered nitrogen-containing heterocyclyl group optionally substituted by one substituent selected from C₁₋₃ alkyl and C₃₋₆ cycloalkyl. In an aspect of this embodiment, the 6-membered heterocyclyl group may be piperidinyl (e.g. 3-piperidinyl) optionally substituted by C₃₋₄ cycloalkyl (e.g. cyclobutyl) or the 6-membered heterocyclyl group may be tetrahydropyran, e.g. 4-tetrahydropyranyl (which is preferably unsubstituted).

In an embodiment R² represents: (i) C₁₋₃ alkyl optionally substituted with one or more substituents independently selected from halo (e.g. fluoro), —OH and —OC₁₋₂ alkyl; (ii) C₃₋₆ cycloalkyl; or (iii) C₂₋₄ alkenyl optionally substituted by —OC₁₋₂ alkyl. In a further embodiment, R² represents C₁₋₃ alkyl optionally substituted with one or more substituents independently selected from halo, —OH and —OC₁₋₂ alkyl. In yet a further embodiment, R² represents unsubstituted C₁₋₃ alkyl.

In a particular embodiment R² represents unsubstituted isopropyl or unsubstituted ethyl.

In an embodiment, R³ represents (i) hydrogen; (ii) halo (e.g. bromo); (iii) C₁₋₄ alkyl optionally substituted with one or more substituents independently selected from halo, —OH and —OC₁₋₂ alkyl; (iv) C₃₋₆ cycloalkyl (e.g. cyclopropyl); or (v) —OC₁₋₃ alkyl. In an embodiment when R³ represents optionally substituted C₁₋₄ alkyl, then it represents C₁₋₃ alkyl optionally substituted by one or more fluoro atoms. In an embodiment when R³ represents C₃₋₆ cycloalkyl, then it represents cyclopropyl. In an embodiment when R³ represents —OC₁₋₃ alkyl, then it represents —OC₁₋₂ alkyl (e.g. —OCH₃).

In a particular embodiment, R³ represents hydrogen, bromo, methyl, ethyl, isopropyl —CF₃, —CHF₂, cyclopropyl or methoxy.

The names of the compounds of the present invention were generated according to the nomenclature rules agreed upon by the Chemical Abstracts Service (CAS) using Advanced Chemical Development, Inc., software (ACD/Name product version 10.01; Build 15494, 1 Dec. 2006) or according to the nomenclature rules agreed upon by the International Union of Pure and Applied Chemistry (IUPAC) using Advanced Chemical Development, Inc., software (ACD/Name product version 10.01.0.14105, October 2006). In case of tautomeric forms, the name of the depicted tautomeric form of the structure was generated. The other non-depicted tautomeric form is also included within the scope of the present invention.

### Preparation of the Compounds

In an aspect of the invention, there is provided a process for the preparation of compounds of the invention, where reference here is made to compounds of formula (I) as defined herein.

Compounds of formula (I) may be prepared by:
(i) reaction of a compound of formula (II),

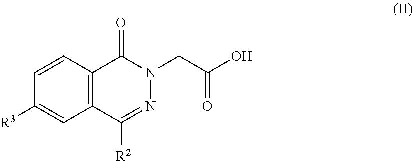

or a derivative thereof (e.g. a salt), wherein R² and R³ are as hereinbefore defined, with a compound of formula (III),

H₂N—R¹ (III)

or a derivative thereof, wherein R¹ is as hereinbefore defined, under amide-forming reaction conditions (also referred to as amidation), for example in the presence of a suitable coupling reagent (e.g. propylphosphonic anhydride, 1-[bis(dimethyl-amino)methylene]-1H-1,2,3-triazolo[4,5-b]pyridinium 3-oxide hexafluorophosphate (O-(7-azabenzotriazol-1-yl)-N,N,N′,N′-tetramethyluronium hexafluorophosphate), 1,1′-carbonyldiimidazole, N,N′-dicyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexa-fluorophosphate (i.e. O-(1H-benzotriazol-1-yl)-N,N,N′,N′-tetramethyluronium hexafluorophosphate), benzotriazol-1-yloxytris-pyrrolidinophosphonium hexa-fluorophosphate, bromo-tris-pyrrolidinophosponium hexafluorophosphate, 2-(1H-benzotriazol-1-yl)-1,1,3,3-tetramethyluronium tetra-fluorocarbonate, 1-cyclohexylcarbodiimide-3-propyloxy-methyl polystyrene, O-benzotriazol-1-yl-N,N,N′,N′-tetramethyluronium tetrafluoroborate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Such reactions may be performed in the presence of a further additive such as 1-hydroxybenzotriazole hydrate. Alternatively, a carboxylic acid group may be converted under standard conditions to the corresponding acyl chloride (e.g. in the presence of SOCl₂ or oxalyl chloride), which acyl chloride is then reacted with a compound of formula (II), for example under similar conditions to those mentioned above;
(ii) reaction of a compound of formula (IV),

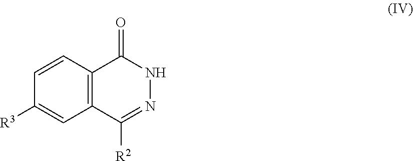

wherein R² and R³ are as hereinbefore defined, with a compound of formula (V),

LG^{a}-CH₂—C(O)—N(H)R¹ (V)

wherein LG^{a} represents a suitable leaving group (e.g. halo, such as chloro) and R¹ is as defined herein, under suitable reaction conditions, e.g. in the presence of an appropriate base, e.g. Cs₂CO₃, K₂CO₃ or LiHMDS, or the like, or alternative alkylation reaction conditions;
(iii) by transformation (such transformation steps may also take place on intermediates) of a certain compound of formula (I) into another, for example:
   for compounds of formula (I) in which R² represents —N(R^{2a})R^{2b}, reaction of a corresponding compound of formula (I) in which R² represents halo, with an appropriate amine HN(R^{2a})R^{2b} (wherein R^{2a} and R^{2b} are as herein defined), in an amination reaction under appropriate conditions, e.g. using under standard coupling conditions, in the presence of a catalyst, e.g. CuI, a ligand, e.g. D/L-proline and a base, e.g. K₂CO₃; similar transformations may be performed on compounds in which another group represents halo, and an amine is desired at another position;
   for compounds of formula (I) containing an alkene, reduction to a corresponding compound of formula (I) containing an alkane, under reduction conditions, e.g. with hydrogen in the presence of a suitable catalyst such as, for example, palladium on carbon, in a suitable reaction-inert solvent, such as, for example, ethanol or methanol;
   coupling to convert a halo or triflate group to e.g. an alkyl, alkenyl or cycloalkyl group, for example in the presence of a suitable coupling reagent, e.g. where the reagent comprises the appropriate alkyl, alkenyl or aryl/heteroaryl group attached to a suitable group such as —B(OH)₂, —B(OR^{WX})₂, zincates (e.g. including —Zn(R^{WX})₂, —ZnBrR^{WX}) or —Sn(R^{WX})₃, in which each R^{WX} independently represents a C₁₋₆ alkyl group, or, in the case of —B(OR^{WX})₂, the respective R^{WX} groups may be linked together to form a 4- to 6-membered cyclic group (such as a 4,4,5,5-tetramethyl-1,3,2-dioxaborolan-2-yl group), thereby forming e.g. a pinacolato boronate ester group. The reaction may be performed in the presence of a suitable catalyst system, e.g. a metal (or a salt or complex thereof) such as Pd, CuI, Pd/C, PdCl₂, Pd(OAc)₂, Pd(Ph₃P)₂Cl₂, Pd(Ph₃P)₄ (i.e. palladium tetrakistriphenyl-phosphine), Pd₂(dba)₃ and/or NiCl₂ (preferred catalysts include RuPhos Pd G3, XPhos Pd and bis(tri-tert-butylphosphine)-palladium(0)) and optionally a ligand such as PdCl₂(dppf).DCM, t-Bu₃P, (C₆H₁₁)₃P, Ph₃P, AsPh₃, P(o-Tol)₃, 1,2-bis(diphenyl-phosphino)ethane, 2,2′-bis(di-tert-butylphosphino)-1,1′-biphenyl, 2,2′-bis(diphenylphosphino)-1,1′-bi-naphthyl, 1,1′-bis(diphenyl-phosphino-ferrocene), 1,3-bis(diphenylphosphino)propane, xantphos, or a mixture thereof, together with a suitable base, such as Na₂CO₃, K₃PO₄, Cs₂CO₃, NaOH, KOH, K₂CO₃, CsF, Et₃N, (i-Pr)₂NEt, t-BuONa or t-BuOK (or mixtures thereof; preferred bases include Na₂CO₃ and K₂CO₃) in a suitable solvent such as dioxane, toluene, ethanol, dimethylformamide, dimethoxyethane, ethylene glycol dimethyl ether, water, dimethylsulfoxide, acetonitrile, dimethylacetamide, N-methylpyrrolidinone, tetrahydrofuran or mixtures thereof (preferred solvents include dimethylformamide and dimethoxyethane);
   reduction of a ketone to an alcohol, in the presence of suitable reducing conditions, e.g. NaBH₄ or the like;
   conversion of a —C(O)alkyl moiety to a —C(OH)(alkyl)(alkyl) moiety by reaction of an appropriate Grignard reagent, e.g. alkylMgBr;
   transformation of a alkene ═CH₂ moiety to a carbonyl ═O moiety, for instance, in the presence of AD-mix-Alpha and methane-sulfonamide, for instance a —CH═CH₂ moiety may be converted to a —C(O)H moiety (e.g. by reaction with osmium tetraoxide), which in turn may be converted to a —CHF₂ group by reaction with DAST;
   transformation of a ketone to an alcohol —OH moiety;
   alkylation of a —OH moiety (to —O-alkyl), under appropriate reaction conditions.

The compound of formula (II) may be prepared by hydrolysis of the corresponding carboxylic acid ester (for example under standard hydrolysis conditions, e.g. base hydrolysis in the presence of an alkali metal hydroxide (such as lithium hydroxide)), which in turn is prepared by reaction of a compound of formula (IV),

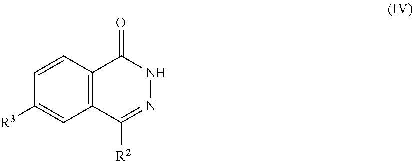

wherein R² and R³ are as hereinbefore defined, with a compound of formula (VI),

LG-CH₂—C(O)O—R^{aa} (VI)

wherein R^{aa} represents C₁₋₆ alkyl (e.g ethyl) and LG represents a suitable leaving group, such as halo (e.g. chloro), for instance under reaction conditions and using reagent such as those described herein.

In general the compounds of the invention can therefore be made with reference to the procedures above. However, in the interests of versatility, further schemes are provided below in order to provide intermediate and final compounds of the invention. Further details are provided in the schemes below (as well as in the specific details of the experimental described hereinafter).

In this respect, Scheme 1 outlines a typical synthesis:

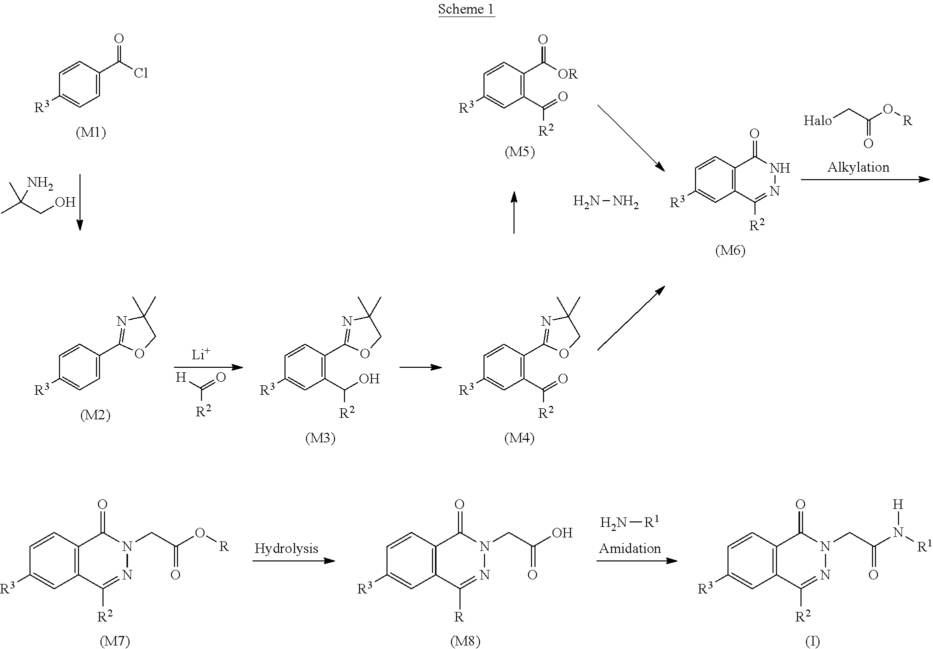

Compounds of the invention, as described herein, can be prepared by a reaction sequence shown in Scheme 1 (above), whereby an appropriate acyl chloride (M1), wherein R³ is as defined herein, is reacted with 2-amino-2-methyl-1-propanol to obtain the corresponding oxazolyl compound (M2), which is reacted with an organometal (e.g. organolithum) to provide a corresponding compound with an ortho-metal substituent (e.g. ortho-lithiated intermediate), which is quenched with an appropriate compound such as an appropriate aldehyde to provide the compound (M3). (M3) is in turn oxidized, e.g. with Dess-Martin reagent, to provide the corresponding ketone (M4). The oxazolyl moiety of (M4) may be hydrolysed to the corresponding ester (M5), e.g. in the presence of a corresponding acid (such as H₂SO₄), however either (M4) or (M5) may be reacted with hydrazine (e.g. in the form of a hydrate) under appropriate conditions to provide compound (M6) (also referred to herein as the compound of formula (IV)). That compound is then alkylated with an appropriate alkyl haloacetate, wherein R is C₁₋₄ alkyl, in the presence of a base, e.g. K₂CO₃, a nucleophilic catalyst, e.g. KI and a crown ether, e.g. 18-crown-6, to provide ester (M7) which is typically cleaved e. g. under basic conditions, e.g. aqueous LiOH in THF or NaOH in MeOH to yield the acid intermediate (M8) (also referred to herein as compound of formula (II)), followed by amidation with R¹—NH₂ (wherein if R¹ has a functional group such as OH, NH₂, CO₂H, such group is optionally protected) using standard coupling conditions, e.g. 1-propanephosphonic anhydride and a base, e.g. triethylamine, optionally followed by an additional deprotection step to provide a compound of Formula (I), or a pharmaceutically acceptable salt thereof.

Further the following transformations, depicted in Schemes 2 and 3 below, show versatility in allowing introduction of other substituents at the R² position of such intermediates too (as well as for final compounds).

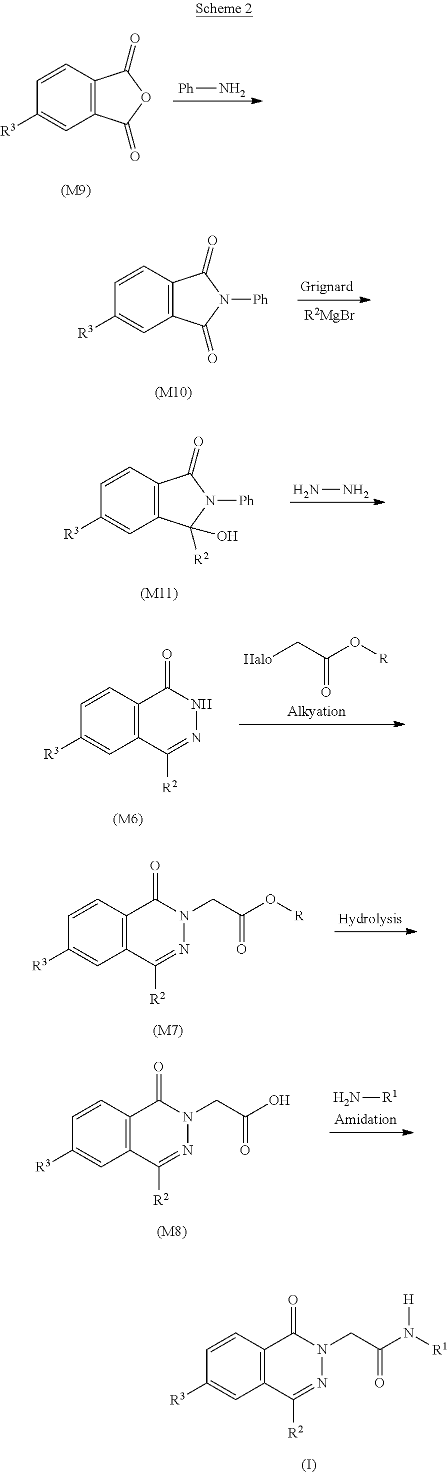

In Scheme 2 (above), an alternative approach to compound (M6) is provided. Starting from (M9), reaction with aniline provides (M10), which may undergo a Grignard reaction to provide (M11)—in this case the Grignard reagent may represent one in which R² represents an appropriate alkyl group—and which intermediate may then undergo reaction with hydrazine (e.g. in the form of a hydrate) to provide (M6). Thereafter transformation may take place for instance in accordance with the procedures outlined by Scheme 1.

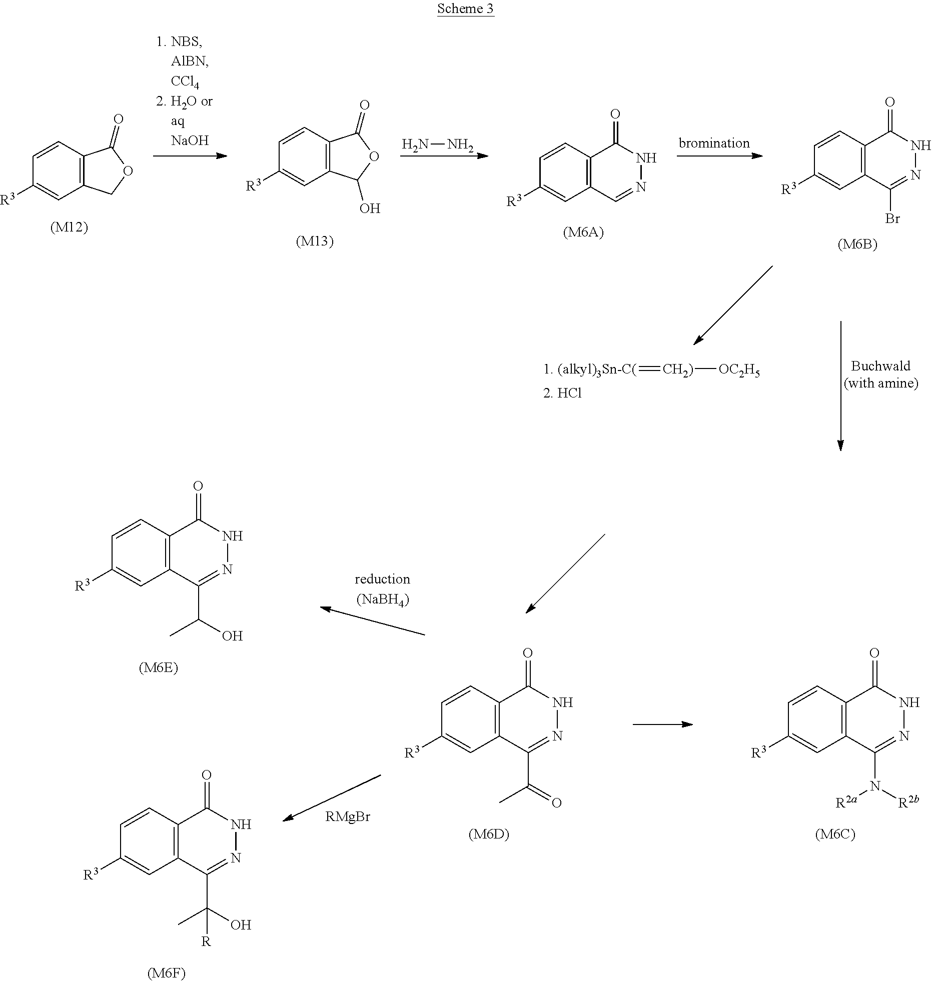

Alternatively, Scheme 3 provides other routes to compounds of formula (IV), also referred to above as compounds (M6). For instance, as per the scheme, compounds of formula (M6A) may undergo bromination to provide a compound of formula (IV) but in which R² represents bromo (M6B). Thereafter further variations of R² groups in downstream products may be obtained. For instance, from (M6B), a Buchwald coupling may provide further compounds such as those of formula (IV) in which R² represents an amino (e.g. —N(R^{2a})(R^{2b}) group, or an another amine group which may be converted to such a group), for instance by reaction in the presence of an amine (e.g. HN(R^{2a})R^{2b}) and an appropriate catalyst (e.g. Pd-based catalyst or another as described herein), optionally with a suitable base and ligand (for example one as described herein, in respect of preparations of compounds of formula (I)). Alternatively, the compound (M6B) may be converted to (M6D), for example in the presence of an appropriate tin-based reagent. That compound (M6D) may then be further converted to either (M6E) or (M6F) by reduction or Grignard reaction, providing alternative R² groups, e.g. optionally substituted alkyl groups (as depicted).

Certain intermediate compounds may be commercially available, may be known in the literature, or may be obtained either by analogy with the processes described herein, or by conventional synthetic procedures, in accordance with standard techniques, from available starting materials using appropriate reagents and reaction conditions.

Certain substituents on/in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations, nitrations or couplings.

Compounds of the invention may be isolated from their reaction mixtures using conventional techniques (e.g. recrystallisations, where possible under standard conditions).

It will be appreciated by those skilled in the art that, in the processes described above and hereinafter, the functional groups of intermediate compounds may need to be protected by protecting groups.

The need for such protection will vary depending on the nature of the remote functionality and the conditions of the preparation methods (and the need can be readily determined by one skilled in the art). Suitable amino-protecting groups include acetyl, trifluoroacetyl, t-butoxycarbonyl (BOC), benzyloxycarbonyl (CBz), 9-fluorenyl-methyleneoxycarbonyl (Fmoc) and 2,4,4-trimethylpentan-2-yl (which may be deprotected by reaction in the presence of an acid, e.g. HCl in water/alcohol (e.g. MeOH)) or the like. The need for such protection is readily determined by one skilled in the art. For example the a —C(O)O-tert-butyl ester moiety may serve as a protecting group for a —C(O)OH moiety, and hence the former may be converted to the latter for instance by reaction in the presence of a mild acid (e.g. TFA, or the like).

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

The compounds of the invention as prepared in the hereinabove described processes may be synthesized in the form of racemic mixtures of enantiomers which can be separated from one another following art-known resolution procedures. Those compounds of the invention that are obtained in racemic form may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of the invention involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

### Pharmacology

There is evidence for a role of NLRP3-induced IL-1 and IL-18 in the inflammatory responses occurring in connection with, or as a result of, a multitude of different disorders (Menu et al., Clinical and Experimental Immunology, 2011, 166, 1-15; Strowig et al., Nature, 2012, 481, 278-286). NLRP3 mutations have been found to be responsible for a set of rare autoinflammatory diseases known as CAPS (Ozaki et al., J. Inflammation Research, 2015, 8, 15-27; Schroder et al., Cell, 2010, 140: 821-832; Menu et al., Clinical and Experimental Immunology, 2011, 166, 1-15). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1 beta. NLRP3 has also been implicated in a number of autoinflammatory diseases, including pyogenic arthritis, pyoderma gangrenosum and acne (PAPA), Sweet's syndrome, chronic nonbacterial osteomyelitis (CNO), and acne vulgaris (Cook et al., Eur. J. Immunol., 2010, 40, 595-653).

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Braddock et al., Nat. Rev. Drug Disc. 2004, 3, 1-10; Inoue et al., Immunology, 2013, 139, 11-18; Coll et al., Nat. Med. 2015, 21(3), 248-55; Scott et al., Clin. Exp. Rheumatol. 2016, 34(1), 88-93), systemic lupus erythematosus and its complications such as lupus nephritis (Lu et al., J. Immunol., 2017, 198(3), 1119-29), and systemic sclerosis (Artlett et al., Arthritis Rheum. 2011, 63(11), 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 2014, 184: 42-54; Kim et al., Am. J. Respir. Crit. Care Med, 2017, 196(3), 283-97). NLRP3 has also been suggested to have a role in a number of central nervous system conditions, including Multiple Sclerosis (MS), Parkinson's disease (PD), Alzheimer's disease (AD), dementia, Huntington's disease, cerebral malaria, brain injury from pneumococcal meningitis (Walsh et al., Nature Reviews, 2014, 15, 84-97; and Dempsey et al., Brain. Behav. Immun. 2017, 61, 306-16), intracranial aneurysms (Zhang et al., J Stroke and Cerebrovascular Dis., 2015, 24, 5, 972-9), and traumatic brain injury (Ismael et al., J. Neurotrauma., 2018, 35(11), 1294-1303). NLRP3 activity has also been shown to be involved in various metabolic diseases including type 2 diabetes (T2D) and its organ-specific complications, atherosclerosis, obesity, gout, pseudo-gout, metabolic syndrome (Wen et al., Nature Immunology, 2012, 13, 352-357; Duewell et al., Nature, 2010, 464, 1357-1361; Strowig et al., Nature, 2014, 481, 278-286), and non-alcoholic steatohepatitis (Mridha et al., J. Hepatol. 2017, 66(5), 1037-46). A role for NLRP3 via IL-1 beta has also been suggested in atherosclerosis, myocardial infarction (van Hout et al., Eur. Heart J. 2017, 38(11), 828-36), heart failure (Sano et al., J. Am. Coll. Cardiol. 2018, 71(8), 875-66), aortic aneurysm and dissection (Wu et al., Arteriosc/er. Thromb. Vase. Biol., 2017, 37(4), 694-706), and other cardiovascular events (Ridker et al., N. Engl. J. Med., 2017, 377(12), 1119-31).

Other diseases in which NLRP3 has been shown to be involved include: ocular diseases such as both wet and dry age-related macular degeneration (Doyle et al., Nature Medicine, 2012, 18, 791-798; Tarallo et al., Cell 2012, 149(4), 847-59), diabetic retinopathy (Loukovaara et al., Acta Ophthalmol., 2017, 95(8), 803-8), non-infectious uveitis and optic nerve damage (Puyang et al., Sci. Rep. 2016, 6, 20998); liver diseases including non-alcoholic steatohepatitis (NASH) and acute alcoholic hepatitis (Henao-Meija et al., Nature, 2012, 482, 179-185); inflammatory reactions in the lung and skin (Primiano et al., J Immunol. 2016, 197(6), 2421-33) including contact hypersensitivity (such as bullous pemphigoid (Fang et al., J Dermatol Sci. 2016, 83(2), 116-23)), atopic dermatitis (Niebuhr et al., Allergy, 2014, 69(8), 1058-67), Hidradenitis suppurativa (Alikhan et al., J. Am. Acad. Dermatol., 2009, 60(4), 539-61), and sarcoidosis (Jager et al., Am. J Respir. Crit. Care Med., 2015, 191, A5816); inflammatory reactions in the joints (Braddock et al., Nat. Rev. Drug Disc, 2004, 3, 1-10); amyotrophic lateral sclerosis (Gugliandolo et al., Int. J. Mol. Sci., 2018, 19(7), E1992); cystic fibrosis (lannitti et al., Nat. Commun., 2016, 7, 10791); stroke (Walsh et al., Nature Reviews, 2014, 15, 84-97); chronic kidney disease (Granata et al., PLoS One 2015, 10(3), eoi22272); and inflammatory bowel diseases including ulcerative colitis and Crohn's disease (Braddock et al., Nat. Rev. Drug Disc, 2004, 3, 1-10; Neudecker et al., J. Exp. Med. 2017, 214(6), 1737-52; Lazaridis et al., Dig. Dis. Sci. 2017, 62(9), 2348-56). The NLRP3 inflammasome has been found to be activated in response to oxidative stress. NLRP3 has also been shown to be involved in inflammatory hyperalgesia (Dolunay et al., Inflammation, 2017, 40, 366-86).

Activation of the NLRP3 inflammasome has been shown to potentiate some pathogenic infections such as influenza and Leishmaniasis (Tate et al., Sci Rep., 2016, 10(6), 27912-20; Novias et al., PLOS Pathogens 2017, 13(2), e1006196).

NLRP3 has also been implicated in the pathogenesis of many cancers (Menu et al., Clinical and Experimental Immunology, 2011, 166, 1-15). For example, several previous studies have suggested a role for IL-1 beta in cancer invasiveness, growth and metastasis, and inhibition of IL-1 beta with canakinumab has been shown to reduce the incidence of lung cancer and total cancer mortality in a randomised, double-blind, placebo-controlled trial (Ridker et al., Lancet., 2017, 390(10105), 1833-42). Inhibition of the NLRP3 inflammasome or IL-1 beta has also been shown to inhibit the proliferation and migration of lung cancer cells in vitro (Wang et al., Oncol Rep., 2016, 35(4), 2053-64). A role for the NLRP3 inflammasome has been suggested in myelodysplastic syndromes, myelofibrosis and other myeloproliferative neoplasms, and acute myeloid leukemia (AML) (Basiorka et al., Blood, 2016, 128(25), 2960-75) and also in the carcinogenesis of various other cancers including glioma (Li et al., Am. J. Cancer Res. 2015, 5(1), 442-9), inflammation-induced tumors (Allen et al., J. Exp. Med. 2010, 207(5), 1045-56; Hu et al., PNAS., 2010, 107(50), 21635-40), multiple myeloma (Li et al., Hematology, 2016 21(3), 144-51), and squamous cell carcinoma of the head and neck (Huang et al., J. Exp. Clin. Cancer Res., 2017, 36(1), 116). Activation of the NLRP3 inflammasome has also been shown to mediate chemoresistance of tumor cells to 5-Fluorouracil (Feng et al., J Exp. Clin. Cancer Res., 2017, 36(1), 81), and activation of NLRP3 inflammasome in peripheral nerve contributes to chemotherapy-induced neuropathic pain (Jia et al., Mol. Pain., 2017, 13, 1-11). NLRP3 has also been shown to be required for the efficient control of viruses, bacteria, and fungi.

The activation of NLRP3 leads to cell pyroptosis and this feature plays an important part in the manifestation of clinical disease (Yan-gang et al., Cell Death and Disease, 2017, 8(2), 2579; Alexander et al., Hepatology, 2014, 59(3), 898-910; Baldwin et al., J. Med. Chem., 2016, 59(5), 1691-1710; Ozaki et al., J. Inflammation Research, 2015, 8, 15-27; Zhen et al., Neuroimmunology Neuroinflammation, 2014, 1(2), 60-65; Mattia et al., J. Med. Chem., 2014, 57(24), 10366-82; Satoh et al., Cell Death and Disease, 2013, 4, 644). Therefore, it is anticipated that inhibitors of NLRP3 will block pyroptosis, as well as the release of pro-inflammatory cytokines (e.g. IL-1 beta) from the cell.

Hence, the compounds of the invention, as described herein (e.g. in any of the embodiments described herein, including by the examples, and/or in any of the forms described herein, e.g. in a salt form or free form, etc) exhibit valuable pharmacological properties, e.g. NLRP3 inhibiting properties on the NLRP3 inflammasome pathway e.g. as indicated in vitro tests as provided herein, and are therefore indicated for therapy or for use as research chemicals, e.g. as tool compounds. Compounds of the invention may be useful in the treatment of an indication selected from: inflammasome-related diseases/disorders, immune diseases, inflammatory diseases, auto-immune diseases, or auto-inflammatory diseases, for example, of diseases, disorders or conditions in which NLRP3 signaling contributes to the pathology, and/or symptoms, and/or progression, and which may be responsive to NLRP3 inhibition and which may be treated or prevented, according to any of the methods/uses described herein, e.g. by use or administration of a compound of the invention, and, hence, in an embodiment, such indications may include:
I. Inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity. Examples of inflammation that may be treated or prevented include inflammatory responses occurring in connection with, or as a result of:
   a. a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
   b. a joint condition such as osteoarthritis, systemic juvenile idiopathic arthritis, adult-onset Still's disease, relapsing polychondritis, rheumatoid arthritis, juvenile chronic arthritis, crystal induced arthropathy (e.g. pseudo-gout, gout), or a seronegative spondyloarthropathy (e.g. ankylosing spondylitis, psoriatic arthritis or Reiter's disease);
   c. a muscular condition such as polymyositis or myasthenia gravis;
   d. a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
   e. a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
   f. a vascular condition such as atherosclerosis, Behcet's disease, vasculitides, or Wegener's granulomatosis;
   g. an immune condition, e.g. autoimmune condition, such as systemic lupus erythematosus (SLE), Sjogren's syndrome, systemic sclerosis, Hashimoto's thyroiditis, type I diabetes, idiopathic thrombocytopenia purpura, or Graves disease;
   h. an ocular condition such as uveitis, allergic conjunctivitis, or vernal conjunctivitis;
   i. a nervous condition such as multiple sclerosis or encephalomyelitis;
   j. an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
   k. a renal condition such as mesangial proliferative glomerulonephritis, nephrotic syndrome, nephritis, glomerular nephritis, acute renal failure, uremia, or nephritic syndrome;
   l. a lymphatic condition such as Castleman's disease;
   m. a condition of, or involving, the immune system, such as hyper lgE syndrome, lepromatous leprosy, familial hemophagocytic lymphohistiocytosis, or graft versus host disease;
   n. a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
   o. a cancer, including those cancers listed herein below;
   p. a burn, wound, trauma, haemorrhage or stroke;
   q. radiation exposure;
   r. obesity; and/or
   s. pain such as inflammatory hyperalgesia;
II. Inflammatory disease, including inflammation occurring as a result of an inflammatory disorder, e.g. an autoinflammatory disease, such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD);
III. Immune diseases, e.g. auto-immune diseases, such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barre syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjogren's syndrome, systemic sclerosis a systemic connective tissue disorder, Takayasu's arteritis, temporal arteritis, warm autoimmune hemolytic anemia, Wegener's granulomatosis, alopecia universalis, Beliefs disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, giant cell arteritis, vitiligo or vulvodynia;
IV. Cancer including lung cancer, renal cell carcinoma, non-small cell lung carcinoma (NSCLC), Langerhans cell histiocytosis (LCH), myeloproliferative neoplams (MPN), pancreatic cancer, gastric cancer, myelodysplastic syndrome (MOS), leukaemia including acute lymphocytic leukaemia (ALL) and acute myeloid leukaemia (AML), promyelocytic leukemia (APML, or APL), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
V. Infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), papillomavirus, or SARS-CoV-2) bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes), and prion infections;
VI. Central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, multiple sclerosis, and amyotrophic lateral sclerosis;
VII. Metabolic diseases such as type 2 diabetes (T2D), atherosclerosis, obesity, gout, and pseudo-gout;
VIII. Cardiovascular diseases such as hypertension, ischaemia, reperfusion injury including post-M1 ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, cardiovascular risk reduction (CvRR), and pericarditis including Dressler's syndrome;
IX. Respiratory diseases including chronic obstructive pulmonary disorder (COPD), asthma such as allergic asthma and steroid-resistant asthma, asbestosis, silicosis, nanoparticle induced inflammation, cystic fibrosis, and idiopathic pulmonary fibrosis;
X. Liver diseases including non-alcoholic fatty liver disease (NAFLD) and nonalcoholic steatohepatitis (NASH) including advanced fibrosis stages F3 and F4, alcoholic fatty liver disease (AFLD), and alcoholic steatohepatitis (ASH);
XI. Renal diseases including acute kidney disease, hyperoxaluria, chronic kidney disease, oxalate nephropathy, nephrocalcinosis, glomerulonephritis, and diabetic nephropathy;
XII. Ocular diseases including those of the ocular epithelium, age-related macular degeneration (AMO) (dry and wet), uveitis, corneal infection, diabetic retinopathy, optic nerve damage, dry eye, and glaucoma;
XIII. Skin diseases including dermatitis such as contact dermatitis and atopic dermatitis, contact hypersensitivity, sunburn, skin lesions, hidradenitis suppurativa (HS), other cyst-causing skin diseases, and acne conglobata;
XIV. Lymphatic conditions such as lymphangitis, and Castleman's disease;
XV. Psychological disorders such as depression, and psychological stress;
XVI. Graft versus host disease;
XVII. Bone diseases including osteoporosis, osteopetrosis;
XVIII. Blood disease including sickle cell disease;
XIX. Allodynia including mechanical allodynia; and
XX. Any disease where an individual has been determined to carry a germline or somatic non-silent mutation in NLRP3.

More specifically the compounds of the invention may be useful in the treatment of an indication selected from: inflammasome-related diseases/disorders, immune diseases, inflammatory diseases, auto-immune diseases, or auto-inflammatory diseases, for example, autoinflammatory fever syndromes (e.g., cryopyrin-associated periodic syndrome), sickle cell disease, systemic lupus erythematosus (SLE), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukemias, myelodysplastic syndromes (MOS), myelofibrosis). In particular, autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis).

In particular, compounds of the invention, may be useful in the treatment of a disease or disorder selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). Thus, as a further aspect, the present invention provides the use of a compound of the invention (hence, including a compound as defined by any of the embodiments/forms/examples herein) in therapy. In a further embodiment, the therapy is selected from a disease, which may be treated by inhibition of NLRP3 inflammasome. In another embodiment, the disease is as defined in any of the lists herein. Hence, there is provided any one of the compounds of the invention described herein (including any of the embodiments/forms/examples) for use in the treatment of any of the diseases or disorders described herein (e.g. as described in the aforementioned lists).

### Pharmaceutical Compositions and Combinations

In an embodiment, the invention also relates to a composition comprising a pharmaceutically acceptable carrier and, as active ingredient, a therapeutically effective amount of a compound of the invention. The compounds of the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

In an embodiment, and depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof. The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

In an embodiment, there is provided a combination comprising a therapeutically effective amount of a compound of the invention, according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention, e.g. for use in the treatment of a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention, but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein), for instance, in an embodiment, the combination may be a kit of parts. Such combinations may be referred to as “pharmaceutical combinations”. The route of administration for a compound of the invention as a component of a combination may be the same or different to the one or more other therapeutic agent(s) with which it is combined. The other therapeutic agent is, for example, a chemical compound, peptide, antibody, antibody fragment or nucleic acid, which is therapeutically active or enhances the therapeutic activity when administered to a patient in combination with a compound of the invention.

The weight ratio of (a) the compound according to the invention and (b) the other therapeutic agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The pharmaceutical composition or combination of the present invention can be in unit dosage of about 1-1000 mg of active ingredient(s) for a subject of about 50-70 kg, or about 1-500 mg, or about 1-250 mg, or about 1-150 mg, or about 1-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

As used herein, term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “subject” as used herein, refers to an animal, preferably a mammal, most preferably a human, for example who is or has been the object of treatment, observation or experiment.

The term “therapeutically effective amount” as used herein, means that amount of compound of the invention (including, where applicable, form, composition, combination comprising such compound of the invention) elicits the biological or medicinal response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by NLRP3, or (ii) associated with NLRP3 activity, or (iii) characterised by activity (normal or abnormal) of NLRP3; or (2) reduce or inhibit the activity of NLRP3; or (3) reduce or inhibit the expression of NLRP3. In another non-limiting embodiment, the term “a therapeutically effective amount” refers to the amount of the compound of the present invention that, when administered to a cell, or a tissue, or a non-cellular biological material, or a medium, is effective to at least partially reduce or inhibit the activity of NLRP3; or at least partially reduce or inhibit the expression of NLRP3.

As used herein, the term “inhibit”, “inhibition” or “inhibiting” refers to the reduction or suppression of a given condition, symptom, or disorder, or disease, or a significant decrease in the baseline activity of a biological activity or process. Specifically, inhibiting NLRP3 or inhibiting NLRP3 inflammasome pathway comprises reducing the ability of NLRP3 or NLRP3 inflammasome pathway to induce the production of IL-1 and/or IL-18. This can be achieved by mechanisms including, but not limited to, inactivating, destabilizing, and/or altering distribution of NLRP3.

As used herein, the term “NLRP3” is meant to include, without limitation, nucleic acids, polynucleotides, oligonucleotides, sense and anti-sense polynucleotide strands, complementary sequences, peptides, polypeptides, proteins, homologous and/or orthologous NLRP molecules, isoforms, precursors, mutants, variants, derivatives, splice variants, alleles, different species, and active fragments thereof.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “pharmaceutical combination” as used herein means a product that results from the mixing or combining of more than one therapeutic agent and includes both fixed and non-fixed combinations of the therapeutic agents. The term “pharmaceutical combination” as used herein refers to either a fixed combination in one dosage unit form, or non-fixed combination or a kit of parts for the combined administration where two or more therapeutic agents may be administered independently at the same time or separately within time intervals. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Summary of Pharmacology, Uses, Compositions and Combinations

In an embodiment, there is provided a pharmaceutical composition comprising a therapeutically effective amount of a compound of the invention, according to any one of the embodiments described herein, and a pharmaceutically acceptable carrier (including one or more pharmaceutically acceptable carriers).

In an embodiment, there is provided a compound of the invention, according to any one of the embodiments described herein, for use as a medicament.

In an embodiment, there is provided a compound of the invention, according to any one of the embodiments described herein (and/or pharmaceutical compositions comprising such compound of the invention, according to any one of the embodiment described herein) for use: in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided a use of compounds of the invention, according to any one of the embodiments described herein (and/or pharmaceutical compositions comprising such compound of the invention, according to any one of the embodiment described herein): in the treatment of a disease or disorder associated with NLRP3 activity (including inflammasome activity); in the treatment of a disease or disorder in which the NLRP3 signalling contributes to the pathology, and/or symptoms, and/or progression, of said disease/disorder; in inhibiting NLRP3 inflammasome activity (including in a subject in need thereof); and/or as an NLRP3 inhibitor.

In an embodiment, there is provided use of compounds of the invention, according to any one of the embodiments described herein (and/or pharmaceutical compositions comprising such compound of the invention, according to any one of the embodiment described herein), in the manufacture of a medicament for: the treatment of a disease or disorder associated with NLRP3 activity (including inflammasome activity); the treatment of a disease or disorder in which the NLRP3 signalling contributes to the pathology, and/or symptoms, and/or progression, of said disease/disorder; and/or inhibiting NLRP3 inflammasome activity (including in a subject in need thereof).

In an embodiment, there is provided a method of treating a disease or disorder in which the NLRP3 signalling contributes to the pathology, and/or symptoms, and/or progression, of said disease/disorder, comprising administering a therapeutically effective amount of a compound of the invention, according to any one of the embodiments described herein (and/or pharmaceutical compositions comprising such compound of the invention, according to any one of the embodiment described herein), for instance to a subject (in need thereof). In a further embodiment, there is provided a method of inhibiting the NLRP3 inflammasome activity in a subject (in need thereof), the method comprising administering to the subject in need thereof a therapeutically effective amount of a compound of the invention, according to any one of the embodiments described herein (and/or pharmaceutical compositions comprising such compound of the invention, according to any one of the embodiment described herein).

In all relevant embodiment of the invention, where a disease or disorder is mentioned (e.g. hereinabove), for instance a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, then such disease may include inflammasome-related diseases or disorders, immune diseases, inflammatory diseases, auto-immune diseases, or auto-inflammatory diseases. In a further embodiment, such disease or disorder may include autoinflammatory fever syndromes (e.g cryopyrin-associated periodic syndrome), liver related diseases/disorders (e.g. chronic liver disease, viral hepatitis, non-alcoholic steatohepatitis (NASH), alcoholic steatohepatitis, and alcoholic liver disease), inflammatory arthritis related disorders (e.g. gout, pseudogout (chondrocalcinosis), osteoarthritis, rheumatoid arthritis, arthropathy e.g acute, chronic), kidney related diseases (e.g. hyperoxaluria, lupus nephritis, Type I/Type II diabetes and related complications (e.g. nephropathy, retinopathy), hypertensive nephropathy, hemodialysis related inflammation), neuroinflammation-related diseases (e.g. multiple sclerosis, brain infection, acute injury, neurodegenerative diseases, Alzheimer's disease), cardiovascular/metabolic diseases/disorders (e.g. cardiovascular risk reduction (CvRR), hypertension, atherosclerosis, Type I and Type II diabetes and related complications, peripheral artery disease (PAD), acute heart failure), inflammatory skin diseases (e.g. hidradenitis suppurativa, acne), wound healing and scar formation, asthma, sarcoidosis, age-related macular degeneration, and cancer related diseases/disorders (e.g. colon cancer, lung cancer, myeloproliferative neoplasms, leukaemia, myelodysplastic syndromes (MOS), myelofibrosis). In a particular aspect, such disease or disorder is selected from autoinflammatory fever syndromes (e.g. CAPS), sickle cell disease, Type I/Type II diabetes and related complications (e.g. nephropathy, retinopathy), hyperoxaluria, gout, pseudogout (chondrocalcinosis), chronic liver disease, NASH, neuroinflammation-related disorders (e.g. multiple sclerosis, brain infection, acute injury, neurodegenerative diseases, Alzheimer's disease), atherosclerosis and cardiovascular risk (e.g. cardiovascular risk reduction (CvRR), hypertension), hidradenitis suppurativa, wound healing and scar formation, and cancer (e.g. colon cancer, lung cancer, myeloproliferative neoplasms, leukemias, myelodysplastic syndromes (MOS), myelofibrosis). In a particular embodiment, the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

In an embodiment, there is provided a combination comprising a therapeutically effective amount of a compound of the invention, according to any one of the embodiments described herein, and another therapeutic agent (including one or more therapeutic agents). In a further embodiment, there is provided such a combination wherein the other therapeutic agent is selected from (and where there is more than one therapeutic agent, each is independently selected from): farnesoid X receptor (FXR) agonists; anti-steatotics; anti-fibrotics; JAK inhibitors; checkpoint inhibitors including anti-PD1 inhibitors, anti-LAG-3 inhibitors, anti-TIM-3 inhibitors, or anti-POL 1 inhibitors; chemotherapy, radiation therapy and surgical procedures; urate-lowering therapies; anabolics and cartilage regenerative therapy; blockade of IL-17; complement inhibitors; Bruton's tyrosine Kinase inhibitors (BTK inhibitors); Toll Like receptor inhibitors (TLR7/8 inhibitors); CAR-T therapy; anti-hypertensive agents; cholesterol lowering agents; leukotriene A4 hydrolase (LTAH4) inhibitors; SGLT2 inhibitors; 132-agonists; anti-inflammatory agents; nonsteroidal anti-inflammatory drugs (“NSAIDs”); acetylsalicylic acid drugs (ASA) including aspirin; paracetamol; regenerative therapy treatments; cystic fibrosis treatments; or atherosclerotic treatment. In a further embodiment, there is also provided such (a) combination(s) for use as described herein in respect of compounds of the invention, e.g. for use in the treatment of a disease or disorder in which the NLRP3 signalling contributes to the pathology, and/or symptoms, and/or progression, of said disease/disorder, or, a disease or disorder associated with NLRP3 activity (including NLRP3 inflammasome activity), including inhibiting NLRP3 inflammasome activity, and in this respect the specific disease/disorder mentioned herein apply equally here. There may also be provided methods as described herein in respect of compounds of the invention, but wherein the method comprises administering a therapeutically effective amount of such combination (and, in an embodiment, such method may be to treat a disease or disorder mentioned herein in the context of inhibiting NLRP3 inflammasome activity). The combinations mentioned herein may be in a single preparation or they may be formulated in separate preparations so that they can be administered simultaneously, separately or sequentially. Thus, in an embodiment, the present invention also relates to a combination product containing (a) a compound according to the invention, according to any one of the embodiments described herein, and (b) one or more other therapeutic agents (where such therapeutic agents are as described herein), as a combined preparation for simultaneous, separate or sequential use in the treatment of a disease or disorder associated with inhibiting NLRP3 inflammasome activity (and where the disease or disorder may be any one of those described herein).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

For instance, compounds of the invention may have the advantage that they have a good or an improved thermodynamic solubility (e.g. compared to compounds known in the prior art; and for instance as determined by a known method and/or a method described herein). Compounds of the invention may have the advantage that they will block pyroptosis, as well as the release of pro-inflammatory cytokines (e.g. IL-1β) from the cell. Compounds of the invention may also have the advantage that they avoid side-effects, for instance as compared to compounds of the prior art, which may be due to selectivity of NLRP3 inhibition. Compounds of the invention may also have the advantage that they have good or improved in vivo pharmacokinetics and oral bioavailability. They may also have the advantage that they have good or improved in vivo efficacy. Specifically, compounds of the invention may also have advantages over prior art compounds when compared in the tests outlined hereinafter (e.g. in Examples C and D).

### General Preparation and Analytical Processes

The compounds according to the invention can generally be prepared by a succession of steps, each of which may be known to the skilled person or described herein.

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SFC).

The starting materials and the intermediates are compounds that are either commercially available or may be prepared according to conventional reaction procedures generally known in the art.

### Analytical Part

### LC-MS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

Table: LCMS Method codes (Flow expressed in m/min; column temperature (T) in ° C.; Run time in minutes).

| Method | | | | | Flow | Run |
|---|---|---|---|---|---|---|
| code | Instrument | column | mobile phase | gradient | Col T | time |
| Method 1 | Agilent | YMC-pack | A: 0.1% HCOOH in H₂O | From 95% A to | 2.6 | 6.2 |
| | 1100 HPLC | ODS-AQ C18 | B: CH₃CN | 5% A in 4.8 min, | 35 | |
| | DAD LC/MS | (50 × 4.6 mm, | | held for 1.0 min, | | |
| | G1956A | 3 μm) | | to 95% A in 0.2 min. | | |
| Method 2 | Waters: Acquity ® | Waters: BEH | A: 10 mM NH₄HCO₃ in | From 100% A to | 0.6 | 3.5 |
| | UPLC ® - DAD | (1.8 μm, | 95% H₂O + 5% CH₃CN | 5% A in 2.10 min, | 55 | |
| | and SQD2 | 2.1*100 mm) | B: MeOH | to 0% A in 1.4 min | | |
| Method 3 | Waters: Acquity ® | Waters: BEH | A: 10 mM CH₃COONH₄ in | From 100% A to | 0.6 | 3.5 |
| | UPLC ® - DAD | (1.8 μm, | 95% H₂O + 5% CH₃CN | 5% A in 2.10 min, | 55 | |
| | and SQD | 2.1*100 mm) | B: CH₃CN | to 0% A in0.9 min, | | |
| | | | | to 5% A in 0.5 min | | |
| Method 4 | Waters: Acquity ® | Waters: BEH | A: 10 mM NH₄HCO₃ in | From 100% A to | 0.6 | 3.5 |
| | UPLC ® - DAD | (1.8 μm, | 95% H₂O + 5% CH₃CN | 5% A in 2.10 min, | 55 | |
| | and SQD | 2.1*100 mm) | B: CH₃CN | to 0% A in 0.9 min, | | |
| | | | | to 5% A in 0.4 min | | |
| Method 5 | Waters: Acquity ® | Waters: BEH | A: 95% CH₃COONH₄ | From 95% A to | 1 | 5 |
| | IClass UPLC ® - | C18 (1.7 μm, | 6.5 mM + 5% CH₃CN, | 5% A in 4.6 min, | 50 | |
| | DAD and Xevo | 2.1 × 50 mm) | B: CH₃CN | held for 0.4 min | | |
| | G2-S QTOF | | | | | |
| Method 6 | Waters: Acquity ® | Waters: BEH | A: 10 mM CH₃COONH₄ in | From 100% A to | 0.6 | 3.5 |
| | UPLC ® - DAD | (1.8 μm, | 95% H₂O + 5% CH₃CN | 5% A in 2.10 min, | 55 | |
| | and SQD2 | 2.1*100 mm) | B: MeOH | to 0% A in 1.4 min | | |
| Method 7 | Waters: Acquity ® | Waters: BEH | A: 95% CH₃COONH₄ | From 95% A to | 0.8 | 2.5 |
| | UPLC ® - DAD | C18 (1.7 μm, | 6.5 mM + 5% CH₃CN, | 5% A in 2.0 min, | 50 | |
| | and SQD2 | 2.1 × 50 mm) | B: CH₃CN | held for 0.5 min | | |
| Method 8 | Waters: Acquity ® | Waters: BEH | A: 95% CH₃COONH₄ | From 95% A to | 1 | 2 |
| | IClass UPLC ® - | C18 (1.7 μm, | 6.5 mM + 5% CH₃CN, | 40% A in 1.2 min, | 50 | |
| | DAD and Xevo | 2.1 × 50 mm) | B: CH₃CN | to 5% A in 0.6 min, | | |
| | G2-S QTOF | | | held for 0.2 min | | |

### NMR

For a number of compounds, ¹H NMR spectra were recorded on a Bruker Avance III spectrometer operating at 300 or 400 MHz, on a Bruker Avance III-HD operating at 400 MHz, on a Bruker Avance NEO spectrometer operating at 400 MHz, on a Bruker Avance Neo spectrometer operating at 500 MHz, or on a Bruker Avance 600 spectrometer operating at 600 MHz, using CHLOROFORM-d (deuterated chloroform, CDCl₃), DMSO-d₆ (deuterated DMSO, dimethyl-d6 sulfoxide), METHANOL-d₄ (deuterated methanol), BENZENE-d₆ (deuterated benzene, C₆D₆) or ACETONE-d₆ (deuterated acetone, (CD₃)₂CO) as solvents. Chemical shifts (□) are reported in parts per million (ppm) relative to tetramethylsilane (TMS), which was used as internal standard.

### Melting Points

Values are either peak values or melt ranges, and are obtained with experimental uncertainties that are commonly associated with this analytical method.

Method A: For a number of compounds, melting points were determined in open capillary tubes on a Mettler Toledo MP50. Melting points were measured with a temperature gradient of 10° C./minute. Maximum temperature was 300° C. The melting point data was read from a digital display and checked from a video recording system

Method B: For a number of compounds, melting points were determined with a DSC823e (Mettler Toledo) apparatus. Melting points were measured with a temperature gradient of 10° C./minute. Standard maximum temperature was 300° C.

### Experimental Part

Hereinafter, the term “m.p.” means melting point, “aq.” means aqueous, “r.m.” means reaction mixture, “rt” means room temperature, ‘DIPEA’ means N,N-diiso-propylethylamine, “DIPE” means diisopropylether, ‘THF’ means tetrahydrofuran, ‘DMF’ means dimethylformamide, ‘DCM’ means dichloromethane, “EtOH” means ethanol ‘EtOAc’ means ethyl acetate, “AcOH” means acetic acid, “iPrOH” means isopropanol, “iPrNH₂” means isopropylamine, “MeCN” or “ACN” means acetonitrile, “MeOH” means methanol, “Pd(OAc)₂” means palladium(II)diacetate, “rac” means racemic, ‘sat.’ means saturated, ‘SFC’ means supercritical fluid chromatography, ‘SFC-MS’ means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rₜ” (or “RT”) means retention time (in minutes), “[M+H]+” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate (1-[bis(dimethylamino)methylene]-1H-1,2,3-triazolo[4,5-b]pyridinium 3-oxide hexafluorophosphate), “Xantphos” means (9,9-dimethyl-9H-xanthene-4,5-diyl)bis[diphenylphosphine], “TBAT” means tetrabutyl ammonium triphenyldifluorosilicate, “TFA” means trifuoroacetic acid, “Et₂O” means diethylether, “DMSO” means dimethylsulfoxide, “SiO₂” means silica, “XPhos Pd G3” means (2-dicyclohexylphosphino-2′,4′,6′-triisopropyl-1,1′-biphenyl)[2-(2′-amino-1,1′-biphenyl)]palladium(II) ethanesulfonate, “CDCl₃” means deuterated chloroform, “MW” means microwave or molecular weight, “min” means minutes, “h” means hours, “rt” means room temperature, “quant” means quantitative, “n.t.” means not tested, “Cpd” means compound, “POCl₃” means phosphorus(V) oxychloride.

For key intermediates, as well as some final compounds, the absolute configuration of chiral centers (indicated as R and/or S) were established via comparison with samples of known configuration, or the use of analytical techniques suitable for the determination of absolute configuration, such as VCD (vibrational circular dichroism) or X-ray crystallography. When the absolute configuration at a chiral center is unknown, it is arbitrarily designated R*.

### Examples—Example A

### Preparation of Intermediates

### Synthesis of 6-chloro-1-methyl-1H-pyrazolo[3,4-b]pyridine I-133

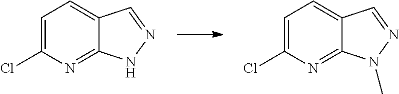

To a solution of 6-chloro-1H-pyrazolo[3,4-b]pyridine [63725-51-9] (1 g, 6.51 mmol) in acetonitrile (10 mL) was added Cs₂CO₃ [534-17-8] (4.24 g, 13 mmol) and the mixture was stirred for 30 min at rt. The reaction mixture was then cooled to 0° C. and iodomethane [74-88-4] (1.63 mL, 2.28 g/mL, 26.15 mmol) was added dropwise. After addition, the reaction mixture was stirred at rt for 1 hour and then heated in the MW at 150° C. for 10 minutes and then left stirring at rt for two days. The reaction was quenched with ice water and extracted with EtOAc. The combined organic extracts were washed with brine, dried over MgSO₄, filtered and concentrated in vacuo. The crude residue was purified by FCC (Hept/EtOAc 0 to 30%) to give I-133 (500 mg, 46%) as a yellow solid.

### Synthesis of N-(1-methyl-1H-pyrazolo[3,4-b]pyridin-6-yl)acetamide I-132

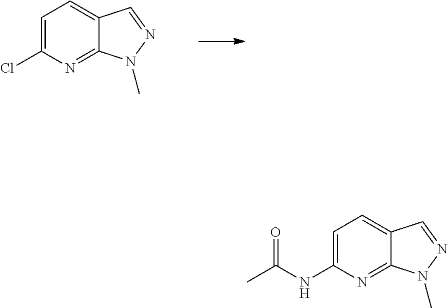

A solution of 6-chloro-1-methyl-1H-pyrazolo[3,4-b]pyridine I-133 (500 mg, 2.98 mmol) in 1,4-dioxane (5 mL) was sparged with nitrogen for 15 min. Acetamide [60-35-5] (212 mg, 3.59 mmol), Pd(OAc)₂ [3375-31-3] (67 mg, 0.3 mmol), xanthphos [161265-03-8] (86.5 mg, 0.15 mmol) and cesium carbonate [534-17-8] (1.95 g, 5.97 mmol) were added. The reaction mixture was heated at 100° C. for 5 hours. The solvent was concentrated under vacuum and the crude mixture was suspended in DCM and filtered. The filtrate was concentrated under vacuum and the obtained solid was purified by FCC (Hept/EtOAc 0 to 20%) to obtain I-132 (458 mg, 81%) as a solid.

### Synthesis of 1-methyl-1H-pyrazolo[3,4-b]pyridin-6-amine I-131

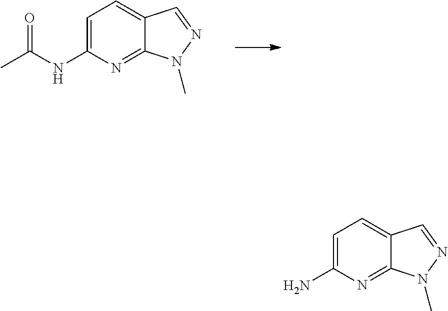

A 37% aqueous solution of HCl [7647-01-0] (23 mL, 1.18 g/mL, 274.8 mmol) was added to a suspension of N-(1-methyl-1H-pyrazolo[3,4-b]pyridin-6-yl)acetamide I-132 (458 mg, 2.41 mmol) in water (25 mL). The resulting solution was heated at reflux for 3 hours. The volatiles were evaporated under vacuum to crude I-131 (444 mg, 100%) as a yellow solid that was used without further purification.

### Synthesis of 5-chloro-3-methyl-3H-imidazo[4,5-b]pyridine I-135 and 5-chloro-1-methyl-1H-imidazo[4,5-b]pyridine I-134

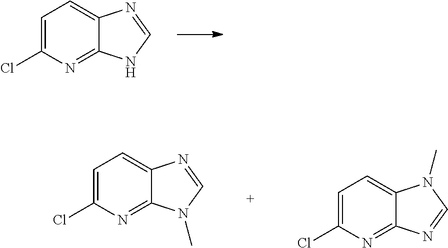

NaH [7646-69-7] (60% dispersion in mineral oil, 1.5 g, 37.5 mmol) was added portionwise to a stirred mixture of 5-chloro-3H-imidazo[4,5-b]pyridine [52090-89-8] (5 g, 32.56 mmol) in anhydrous DMF (70 mL) at 0° C. The resulting mixture was allowed to warm to rt and stirred 30 min before dropwise addition of MeI [74-88-4] (2.3 mL, 2.28 g/mL, 36.945 mmol). After stirring for 2 hours at rt, the mixture was carefully quenched with water. EtOAc and more water were added. The organic layer was separated, washed with brine (×5), dried (MgSO₄), filtered and evaporated under reduced pressure. The crude mixture was purified by FCC (DCM/MeOH 1 to 3%) to afford I-135 (3.7 g, 68%) as a pale orange solid and I-134 (880 mg, 16%) as a white solid.

### Synthesis of 1-ethyl-5-nitroindoline I-148

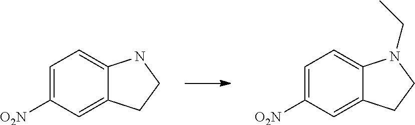

5-nitroindoline [32692-19-6] (3 g, 18.27 mmol) was dissolved in dry DMF (60 mL) while stirring under a nitrogen atmosphere. NaH [7646-69-7] (60% suspension in mineral oil, 1.46 g, 36.55 mmol) was added portionwise at rt over a 10 min period. The reaction mixture was stirred at rt for 20 min. A solution iodoethane [75-03-6] (4.66 g, 29.88 mmol) in DMF (10 mL) was added dropwise over a 10 min period and then the mixture was heated at 75° C. and stirred overnight at this temperature. It was allowed to cool to rt, quenched by addition of water and extracted with EtOAc. The organic extracts were combined and washed with brine, dried over MgSO₄ and evaporated. The residue was purified by FCC (Hept/DCM 0 to 80% yielding I-148 (2.24 g, 64%) as a orange solid.

Structural analogues were synthesized according to the above procedure.

| Alkylating agent | Intermediate |
|---|---|
| 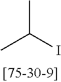 | 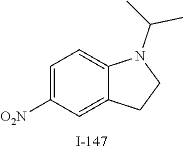 |
| 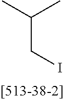 | 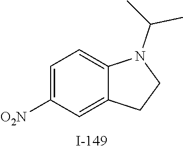 |

### Synthesis of 1-ethylindolin-5-amine I-145

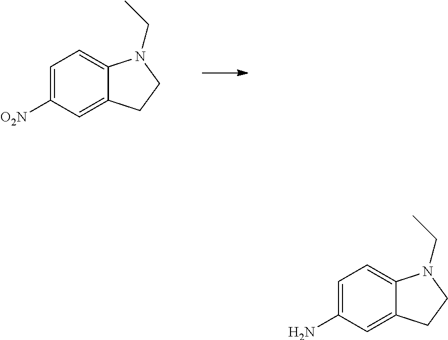

A mixture of 1-ethyl-5-nitroindoline I-148 (1 g, 5.2 mmol) and Pd/C (10% wt. Pd, 0.1 g, 0.094 mmol) in MeOH (25 mL) was placed under hydrogen atmosphere at rt and stirred until uptake of 3 equivalents of hydrogen was observed. The catalyst was filtered and the filtrate concentrated under reduced pressure. The obtained residue was purified by FCC (DCM/MeOH 0 to 5%) yielding I-145 (440 mg, 52%) as a purple oil.

| Substrate | Intermediate |
|---|---|
| 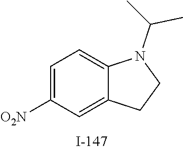 | 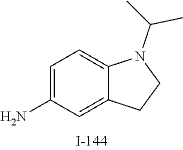 |
| 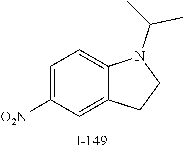 | 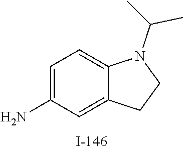 |

### Synthesis of 1-(difluoromethyl)-6-nitro-1H-indazole I-157

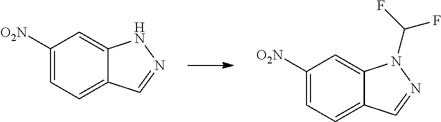 [SMILES: O=[N+]([O-])c1ccc2cn[nH]c2c1 ; O=[N+]([O-])c1ccc2cnn(C(F)F)c2c1]

NaH [7646-69-7] (60% dispersion in mineral oil, 0.1 g, 2.54 mmol) was added to a solution of 6-nitro-2H-indazole [65750-02-9] (500 mg, 3.06 mmol) and sodium chlorodifluoroacetate [1895-39-2] (0.78 g, 5.09 mmol) in N-methylpyrrolidinone (8.5 ml). Thereafter, the reaction mixture was stirred at rt for 15 min, and further stirred at 100° C. for 30 min. It was diluted with ethyl acetate, and washed successively with water and brine. The combined organic extracts were dried over MgSO₄, filtered and the solvent was evaporated under vacuum. The residue was purified by FCC (Hept/EtOAc 0 to 20%) to obtain I-157 (130 mg, 20%) as a solid.

### Synthesis of 1-(difluoromethyl)-1H-indazol-6-amine I-156

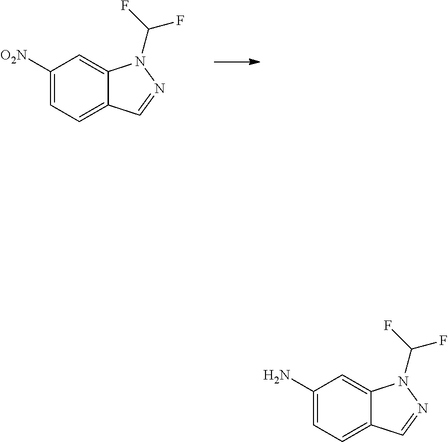 [SMILES: O=[N+]([O-])c1ccc2cnn(C(F)F)c2c1 ; Nc1ccc2cnn(C(F)F)c2c1]

Pd/C (10% wt. Pd, 68.09 mg, 0.064 mmol) was added to a solution of I-157 (120 mg, 0.56 mmol) in ethyl acetate (5 mL) in a 100-mL hydrogenation flask. The reaction was purged three times (hydrogen/vacuum) and placed under hydrogen atmosphere. The reaction was stirred overnight at rt. It was filtered over Decalite, washing thoroughly with EtOAc and the solvent was concentrated under reduced pressure to afford I-156 (103 mg, 100%) as a pale pink solid.

### Synthesis of 1-(difluoromethyl)-1H-indazol-5-amine I-158

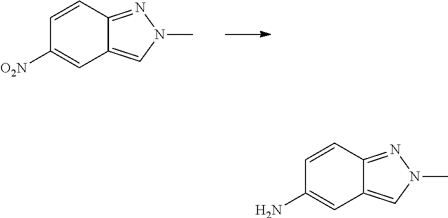

Iron powder [7439-89-6] (513.4 mg, 9.19 mmol) was added to a mixture of 2-methyl-5-nitro-2H-indazole [5228-48-8] (228.5 mg, 1.29 mmol), ammonium chloride [12125-02-9] (209.5 mg, 3.92 mmol) in a mixture of EtOH (11 mL) and DI water (11 mL) in a MW vial. The vial was sealed and the reaction mixture was stirred vigorously and heated at 100° C. for 1 hour. The crude mixture was filtered over Celite and the cake was washed with EtOAc (˜30 mL. The filtrate was filtered again over a millipore filter and the cake was washed with EtOAc (˜20 mL). The filtrate was washed with water (˜10 mL), dried over MgSO₄ and concentrated under reduced pressure at 50° C. to afford I-158 (193 mg, 97%) as a brown oil which was used in the next step without further purification.

### Synthesis of 3-bromo-2-hydrazineyl-5-nitropyridine I-161

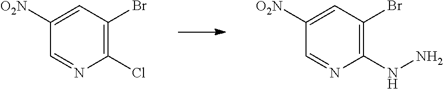 [SMILES: O=[N+]([O-])c1cnc(Cl)c(Br)c1 ; NNc1ncc([N+](=O)[O-])cc1Br]

3-Bromo-2-chloro-5-nitropyridine [5470-17-7] (1.5 g, 6.32 mmol) was dissolved in 1,4-dioxane (81 mL), the solution cooled to 0° C. and hydrazine hydrate [7803-57-8](9.2 mL, 1.03 g/mL, 0.19 mol) was added quickly (<15 seconds) at 0° C. After addition, the mixture was stirred vigorously at 0° C. for 1.5 hours then allowed to warm to r.t. and stirred for a further hour. The mixture was concentrated on a rotary evaporator to about 20 mL of dark red mixture. It was then cooled to 0° C. and DI water (150 mL) was added. A solid precipitated and was filtered off on a sinter funnel, washing the flask and solid with ca. 5+5 mL of DI water. After drying in the oven at 50° C. under vacuum for 16 hours, I-161 (1.21 g, 82%, ca. 96-97% purity) was isolated as a greyish solid.

### Synthesis of 8-bromo-6-nitro-[1,2,4]triazolo[4,3-a]pyridine I-160

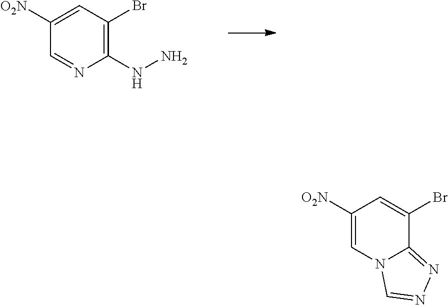 [SMILES: NNc1ncc([N+](=O)[O-])cc1Br ; O=[N+]([O-])c1cc(Br)c2nncn2c1]

3-Bromo-2-hydrazinyl-5-nitropyridine I-161 (4 g, 17.2 mmol) was suspended in trimethyl orthoformate [149-73-5] (28.2 mL, 0.97 g/mL, 0.26 mol) in an EasyMax pressure tube. The tube was sealed with a screw-cap and the mixture heated at 100° C. for 2.5 hours. The reaction was allowed to cool to rt, then cooled to 0° C. for ca. 30 min and the suspension was filtered off washing the reaction vial and filtered solid with a 1:1 mixture of Heptane/EtOAc (10 mL) to yield I-160 (3.66 g, >98% purity, 88%) as a pale brown solid.

### Synthesis of 8-bromo-[1,2,4]triazolo[1,5-a]pyridin-6-amine I-159

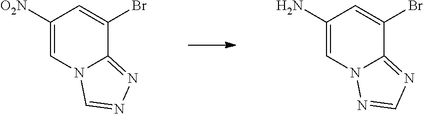 [SMILES: O=[N+]([O-])c1cc(Br)c2nncn2c1 ; Nc1cc(Br)c2ncnn2c1]

8-bromo-6-nitro-[1,2,4]triazolo[4,3-a]pyridine pyridine I-160 (500 mg, 2.06 mmol), NH₄Cl [12125-02-9] (880.4 mg, 16.5 mmol) and iron powder [7439-89-6] (804.3 mg, 14.4 mmol) were placed in a screw-cap vial equipped with a magnetic stir bar and suspended in EtOH (8 mL). The suspension was stirred vigorously and heated at 85° C. for 64 hours. The suspension was filtered on a sinter funnel and the filtrate concentrated in vacuo, retaken in MeCN (ca. 20 mL) and concentrated again to give a tan solid (423 mg, contains large amount of salts/iron). 147 mg of this material was partitioned between a saturated aqueous solution of NaHCO₃aq. (50 mL) and DCM/MeOH 95:5 (25 mL). The organic layer was collected and the aqueous layer re-extracted with DCM/MeOH 95:5 (15+10 mL). The combined organic layers were concentrated under reduced pressure to give I-159 (78 mg, 18%) as a greenish solid.

### Synthesis of 8-bromo-[1,2,4]triazolo[4,3-a]pyridin-6-amine I-162

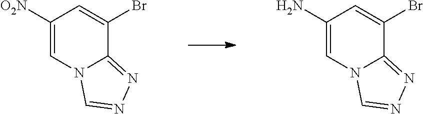 [SMILES: O=[N+]([O-])c1cc(Br)c2nncn2c1 ; Nc1cc(Br)c2nncn2c1]

8-Bromo-6-nitro-[1,2,4]triazolo[4,3-a]pyridine I-160 (1 g, 4.11 mmol, 1 equiv) and iron powder [7439-89-6] (1.38 g, 24.7 mmol) were placed in a screw-cap tube and AcOH [64-19-7] (18.8 mL) was added. The mixture was stirred vigorously at rt for 3 hours. The green thick suspension was diluted with DI water (30-40 mL). This thus obtained dark mixture was concentrated in vacuo down to ca. 10 mL of volume left. The residue was neutralised by slow addition of 80 mL of a 1:1 mixture of saturated aqueous NaHCO₃ and K₂CO₃ (effervescence ceased after addition of ca. 10-15 mL, then a solid formed that redissolved upon addition of more basic solution). The mixture was then extracted with DCM/MeOH 95:5 (5×150 mL). The combined organic extracts were dried over Na₂SO₄, filtered and the filtrate concentrated in vacuo to afford I-162 (450 mg, 51%) as a pale tan solid.

### Synthesis of tert-butyl (5-hydroxy-1-methylpiperidin-3-yl)carbamate I-164

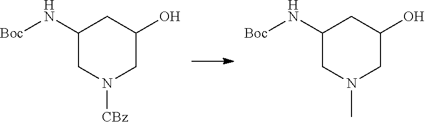

A mixture of benzyl 3-((tert-butoxycarbonyl)amino)-5-hydroxypiperidine-1-carboxylate [1785642-46-7] (1 g, 2.85 mmol) and Pd/C (10% wt. Pd, 3.04 g, 2.85 mmol) in MeOH (20 ml) was hydrogenated at rt under atmospheric pressure of hydrogen until full conversion was observed. The suspension was used in the next step without purification. To this suspension, were added Pd/C (10% wt. Pd, 3.04 g, 2.85 mmol) and paraformaldehyde [30525-89-4] (0.5 g, 16.67 mmol) and the mixture was placed under hydrogen until full conversion was observed. The catalyst was filtered and the filtrate was concentrated in vacuo. The obtained residue was purified by FCC (DCM/MeOH:NH₃ (7N) 0 to 7% yielding I-164 (537 mg, 82%) as a white solid.

### Synthesis of tert-butyl (R)-(1-ethylpiperidin-3-yl)carbamate I-1001

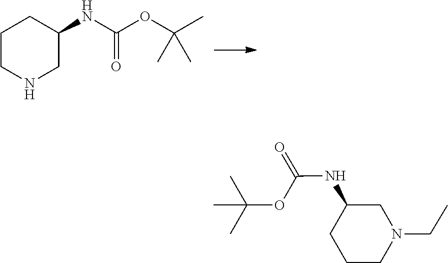 [SMILES: CC(C)(C)OC(=O)N[C@@H]1CCCNC1 ; CCN1CCC[C@@H](NC(=O)OC(C)(C)C)C1]

Sodium triacetoxyborohydride [56553-60-7] (19.84 g, 93.62 mmol) was added portionwise to a mixture of (R)-3-(BOC-amino)piperidine [309956-78-3] (12.5 g, 62.41 mmol) and acetaldehyde, 5 M in THF [75-07-0] (14.98 mL, 5 M, 74.89 mmol). The mixture was stirred at rt for 4 h. Water and NaHCO₃ were added and the mixture mixture was extracted with DCM. The combined organic extracts were dried over MgSO4, filtered and concentrated in vacuum. The crude was purified by flash column chromatography (silica; MeOH—NH3 in DCM, 0 to 4%). The desired fractions were collected and concentrated in vacuo to yield tert-butyl (R)-(1-ethylpiperidin-3-yl)carbamate I-1001 as a white solid.

### Synthesis of 3-amino-5-hydroxy-1-methylpiperidine hydrochloride I-163

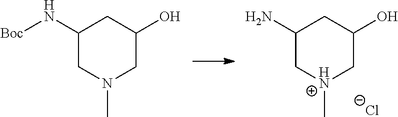

A 4M solution HCl in 1,4-dioxane (6.89 mL, 27.6 mmol) was added to a solution of (5-hydroxy-1-methylpiperidin-3-yl)carbamate I-164 (520 mg, 2.26 mmol) in 1,4-dioxane (6.9 mL). The resulting mixture was stirred at rt for 3 hours. The volatiles were concentrated to yield I-163 (461 mg, quantitative) as a white solid that was used without further purification.

Structural analogues were synthesized according to the above procedure.

| Substrate | Intermediate |
|---|---|
| 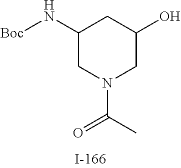 [SMILES: CC(=O)N1CC(O)CC(NC(=O)OC(C)(C)C)C1] | 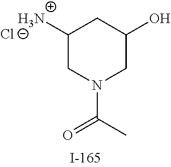 |
| 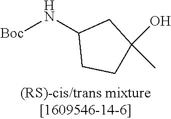 | 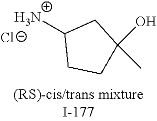 |
| 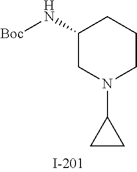 [SMILES: CC(C)(C)OC(=O)N[C@@H]1CCCN(C2CC2)C1] | 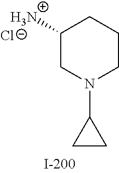 |
| 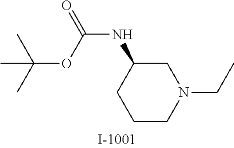 [SMILES: CCN1CCC[C@@H](NC(=O)OC(C)(C)C)C1] | 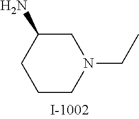 |

### Synthesis of tert-butyl (1-acetyl-5-hydroxypiperidin-3-yl)carbamate I-166

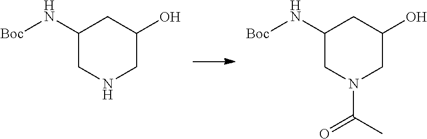 [SMILES: CC(C)(C)OC(=O)NC1CNCC(O)C1 ; CC(=O)N1CC(O)CC(NC(=O)OC(C)(C)C)C1]

Ac₂O [108-24-7] (229 μL, 2.43 mmol) was added dropwise to a solution of tert-butyl N-(5-hydroxypiperidin-3-yl)carbamate [1502766-14-4] (0.5 g, 2.31 mmol) and triethylamine [121-44-8] (417 μL, 3.01 mmol) in dry DCM (10 mL) and the mixture was stirred at rt overnight The reaction mixture was washed with a saturated solution of NaHCO₃, dried over MgSO₄ and evaporated to yield I-165 (525 mg, 88%) as a white foam.

### Synthesis of tert-butyl (R)-(1-cyclopropylpiperidin-3-yl)carbamate I-201

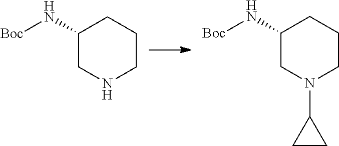 [SMILES: CC(C)(C)OC(=O)N[C@@H]1CCCNC1 ; CC(C)(C)OC(=O)N[C@@H]1CCCN(C2CC2)C1]

Sodium cyanoborohydride [25895-60-7] (667 mg, 10.61 mmol) was added to a suspension of (R)-3-Boc-aminopiperidine [309956-78-3] (1 g, 4.99 mmol), (1-ethoxycyclopropoxy)trimethylsilane [27374-25-0] (1 mL, 4.99 mmol) and acetic acid (3 mL, 52.4 mmol) in MeOH (40 mL) in a pressure tube under nitrogen. The reaction mixture was stirred overnight at 70° C. The crude mixture was purified by preparative HPLC (Stationary phase: RP XBridge Prep C18 OBD-10 μm, 50×250 mm, Mobile phase: 0.25% NH₄HCO₃ solution in water, MeOH) to afford I-201 (600 mg, 50%) as a white solid.

### Synthesis of 3-chloro-1-(difluoromethyl)-5-nitropyridin-2(1H)-one I-168

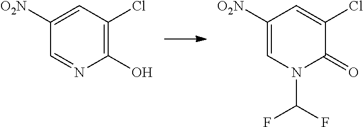 [SMILES: O=[N+]([O-])c1cnc(O)c(Cl)c1 ; O=c1c(Cl)cc([N+](=O)[O-])cn1C(F)F]

A solution of 3-chloro-2-hydroxy-5-nitropyridine [22353-38-4] (2 g, 11.46 mmol) in DMSO (20 mL) was placed in an EasyMax pressure tube under an inert atmosphere of nitrogen at room temperature. NaH [7646-69-7] (60% dispersion in mineral oil, 0.5 g, 12.5 mmol) was added to this mixture which was allowed to react for 15 min at rt. Sodium chlorodifluoroacetate [1895-39-2] (2 g, 13.12 mmol) was added to the mixture and the resulting solution was stirred overnight at 60° C. The reaction mixture was allowed to cool to rt and quenched by addition of DI water. The resulting solution was extracted three times with EtOA.c The combined organic extracts were dried over MgSO₄, filtered and concentrated under vacuum. The residue was purified by FCC (Hept/EtOAc 0 to 40%) to obtain I-168 (390 mg, 15%) as a white solid.

### Synthesis of 5-amino-3-chloro-1-(difluoromethyl)pyridin-2(1H)-one I-167

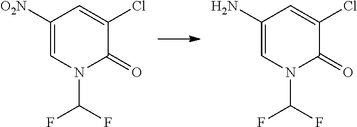 [SMILES: O=c1c(Cl)cc([N+](=O)[O-])cn1C(F)F ; Nc1cc(Cl)c(=O)n(C(F)F)c1]

A mixture of 3-chloro-1-(difluoromethyl)-5-nitropyridin-2(1H)-one I-168 (100 mg, 0.45 mmol), iron powder [7439-89-6] (73.8 mg, 1.32 mmol) and a saturated aqueous solution of ammonium chloride [12125-02-9] (0.42 mL, ca. 7.2 M, ca. 3.01 mmol) in EtOH (1.7 mL) in a sealed MW vial under nitrogen was heated at 80° C. overnight. The reaction mixture was allowed to cool to room temperature and filtered over dicalite, washing thoroughly with EtOH. The filtrate was evaporated under reduced pressure, suspended in DCM and filtered again. The filtrate was concentrated and purified by FCC (DCM/MeOH 0 to 5%) to obtain I-167 (37 mg, 43%) as a dark film.

### Synthesis of 6-chloroimidazo[1,2-b]pyridazine-2-carbaldehyde I-172

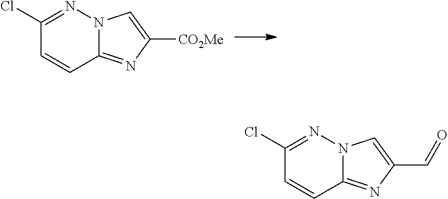 [SMILES: COC(=O)c1cn2nc(Cl)ccc2n1 ; O=Cc1cn2nc(Cl)ccc2n1]

Methyl 6-chloroimidazo[1,2-b]pyridazine-2-carboxylate [572910-59-9] (2 g, 9.45 mmol) was placed in a 100-mL RB flask equipped with a magnetic stir bar. The flask was placed under nitrogen (3 vacuum/nitrogen cycles). Anhydrous DCM (26 mL) was added and the mixture stirred vigorously and cooled to −78° C. Then, a 1M solution of DIBAL in cyclohexane [1191-15-7] (15.6 mL, 15.6 mmol) was added dropwise over 10 minutes. The resulting mixture was stirred at −78° C. for 1 hour. It was then quenched by addition of a saturated aqueous solution of NH₄Cl (35 mL), DI water was added (15 mL) and the mixture was extracted with DCM (5×60 mL). The combined organic extracts were dried over Na₂SO₄, filtered and concentrated in vacuo. The crude solid was purified by FCC (Hept/EtOAc 4:1 to 1:4) to give I-172 (795 mg, 46%) as a colorless solid.

### Synthesis of 6-chloro-2-(difluoromethyl)imidazo[1,2-b]pyridazine I-171

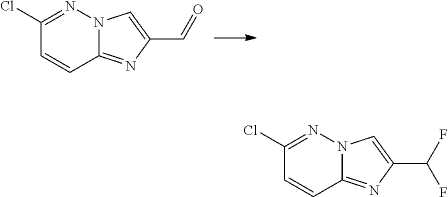 [SMILES: O=Cc1cn2nc(Cl)ccc2n1 ; FC(F)c1cn2nc(Cl)ccc2n1]

6-Chloroimidazo[1,2-b]pyridazine-2-carbaldehyde I-172 (795 mg, 4.38 mmol) was suspended in anhydrous DCM (15 mL), the suspension cooled to 0° C. and DAST [38078-09-0] (1.74 mL, 13.1 mmol) was added dropwise at 0° C. The resulting mixture was stirred at 0° C. for 5 min then allowed to warm to rt and stirred for 2 hours. Another portion of DAST [38078-09-0] (0.87 mL, 6.57 mmol) was added dropwise and the resulting suspension stirred at rt for 16 hours. The crude mixture was cooled to 0° C. and quenched by slow addition of a saturated aqueous solution of NaHCO₃ (50 mL) The biphasic mixture was stirred at rt until effervescence had ceased and the product was extracted with DCM (3×20 mL). The combined organic extracts were dried over Na₂SO₄, filtered and concentrated in vacuo. The obtained residue was purified by FCC (Hept/EtOAc 9:1 to 1:1) to afford I-171 (790 mg, 89%) as a colorless crystalline solid.

### Synthesis of N-(2-(difluoromethyl)imidazo[1,2-b]pyridazin-6-yl)-1,1-diphenylmethanimine I-170

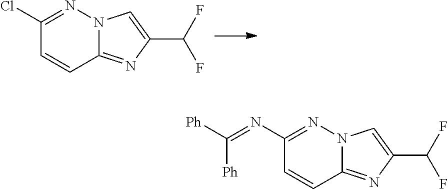 [SMILES: FC(F)c1cn2nc(Cl)ccc2n1 ; FC(F)c1cn2nc(N=C(c3ccccc3)c3ccccc3)ccc2n1]

6-Chloro-2-(difluoromethyl)imidazo[1,2-b]pyridazine I-171 (250 mg, 1.23 mmol), Pd₂dba₃ [51364-51-3] (112.5 mg, 0.12 mmol), BINAP [98327-87-8] (152.9 mg, 0.25 mmol), and NaO^{t}Bu [865-48-5] (188.8 mg, 1.96 mmol) were placed in a 20-mL MW vial. The vial was sealed and placed under nitrogen (3 vacuum/nitrogen cycles). Then, benzophenonimine [1013-88-3] (309 μL, 1.08 g/mL, 1.84 mmol) was added as a solution in degassed 1,4-dioxane [123-91-1] (9 mL). The mixture was then stirred vigorously and heated at 80° C. for 3 hours. The mixture was concentrated in vacuo and the residue purified by FCC (Hept/EtOAc 3:1 to 3:7) to afford I-170 (300 mg, ca. 95% purity, 67%) as an orange solid that was used without further purification.

Structural analogues were synthesized according to the above procedure.

| Substrate | Intermediate |
|---|---|
| 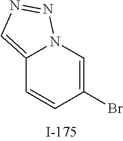 | 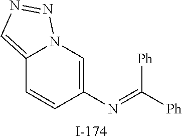 [SMILES: c1ccc(C(=Nc2ccc3cnnn3c2)c2ccccc2)cc1] |
| 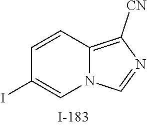 [SMILES: N#Cc1ncn2cc(I)ccc12] | 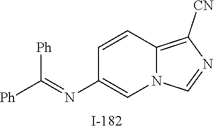 [SMILES: N#Cc1ncn2cc(N=C(c3ccccc3)c3ccccc3)ccc12] |
| 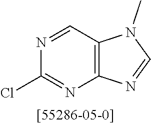 | 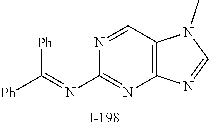 [SMILES: Cn1cnc2nc(N=C(c3ccccc3)c3ccccc3)ncc21] |

### Synthesis of 2-(difluoromethyl)imidazo[1,2-b]pyridazin-6-amine hydrochloride I-169

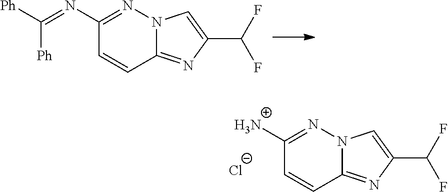 [SMILES: FC(F)c1cn2nc(N=C(c3ccccc3)c3ccccc3)ccc2n1 ; [Cl-] ; [NH3+]c1ccc2nc(C(F)F)cn2n1]

N-(2-(Difluoromethyl)imidazo[1,2-b]pyridazin-6-yl)-1,1-diphenylmethanimine I-170 (300 mg, ca. 95% purity, 67%) was dissolved in THF (5 mL) and a 1M aqueous solution of HCl [7647-01-0] (7.5 mL, 7.5 mmol) was added. The mixture was stirred vigorously at rt for 2 hours. The mixture was concentrated in vacuo at 50° C. and retaken in acetonitrile (3×15 mL) and concentrated (3×) to afford a yellow solid that was triturated with MeCN (1.5 mL) to give I-169 (135 mg, ca. 85-90% purity, ca. 45%) a pale yellow solid that was used without further purification.

Structural analogues were synthesized according to the above procedure.

| Substrate | Intermediate |
|---|---|
| 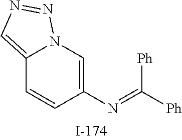 [SMILES: c1ccc(C(=Nc2ccc3cnnn3c2)c2ccccc2)cc1] | 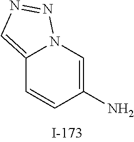 |
| 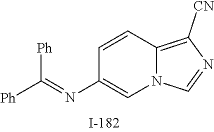 [SMILES: N#Cc1ncn2cc(N=C(c3ccccc3)c3ccccc3)ccc12] | 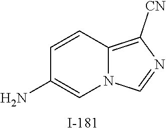 [SMILES: N#Cc1ncn2cc(N)ccc12] |
| 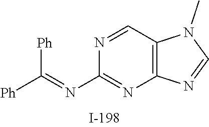 [SMILES: Cn1cnc2nc(N=C(c3ccccc3)c3ccccc3)ncc21] | 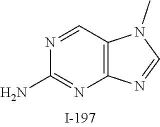 |

### Synthesis of (EZ)-N′-((5-bromopyridin-2-yl)methylene)-4-methylbenzenesulfonohydrazide I-176

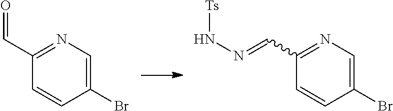 [SMILES: O=Cc1ccc(Br)cn1 ; Cc1ccc(S(=O)(=O)NN=Cc2ccc(Br)cn2)cc1]

4-Methylbenzenesulfonylhydrazide [1576-35-8] (1.0 g, 5.38 mmol) was added to a solution of 5-bromopyridine-2-carbaldehyde [31181-90-5] (1.0 g, 5.376 mmol) in DCM (10 mL) and MeOH (10 mL). The reaction was stirred at rt for 1 hour. Volatiles were removed under reduced pressure and the obtained solid I-176 (1.90 g, quantitative) was used as such in the following step.

### Synthesis of 6-bromo-[1,2,3]triazolo[1,5-a]pyridine I-175

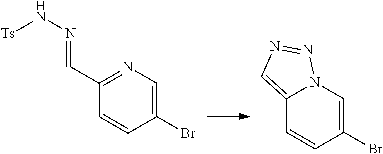 [SMILES: Cc1ccc(S(=O)(=O)N/N=C/c2ccc(Br)cn2)cc1 ; Brc1ccc2cnnn2c1]

A mixture of crude EZ)-N′-((5-bromopyridin-2-yl)methylene)-4-methylbenzenesulfonohydrazide I-176 (1.90 g, 5.36 mmol) and morpholine [110-91-8](10 mL, 115.9 mmol) were stirred at 90° C. for 1 hour. The reaction was cooled to rt and then cooled to 0° C. and treated with DIPE until a precipitate formed. The solid was discarded and the filtrate was evaporated under reduced pressure. The crude product was purified by FCC (Hept/EtOAc 0 to 60%) to obtain I-175 (970 mg, 91%) as a white solid.

### Synthesis of 6-aminoimidazo[1,2-a]pyridine-2-carboxamide I-180

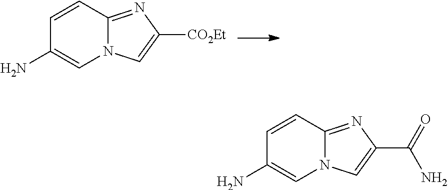 [SMILES: CCOC(=O)c1cn2cc(N)ccc2n1 ; NC(=O)c1cn2cc(N)ccc2n1]

A mixture of ethyl 6-aminoimidazo[1,2-a]pyridine-2-carboxylate [158980-21-3] (1 g, 4.87 mmol) in aqueous ammonia [7664-41-7] (28% in H₂O, 20 mL) was stirred and heated in a pressure tube at 90° C. for 3 hours. Volatiles were evaporated under vacuum and the crude product I-180 (0.86 g, quantitative) was used without any purification in the next step.

### Synthesis of N-(2-cyanoimidazo[1,2-a]pyridin-6-yl)-2,2,2-trifluoroacetamide I-179

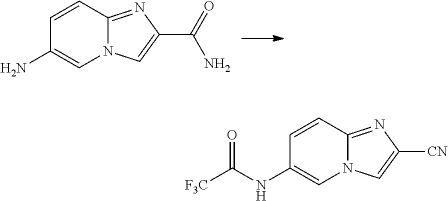 [SMILES: NC(=O)c1cn2cc(N)ccc2n1 ; N#Cc1cn2cc(NC(=O)C(F)(F)F)ccc2n1]

TFAA [407-25-0] (0.28 mL, 1.51 g/mL, 1.99 mmol) was added to a solution of 6-aminoimidazo[1,2-a]pyridine-2-carboxamide I-180 (100 mg, 0.57 mmol) and triethylamine [121-44-8] (0.39 mL, 0.73 g/mL, 2.84 mmol) in dry THF (3 mL) under nitrogen at 0° C. The reaction was stirred at 0° C. for another hour and then at rt for 2 hours. The reaction mixture was quenched by addition of water and extracted with DCM. The combined organic extracts was dried on MgSO₄, filtered and evaporated in vacuo. The obtained solid I-179 (140 mg, quantitative) was used without further purification in the next step.

### Synthesis of 6-aminoimidazo[1,2-a]pyridine-2-carbonitrile I-178

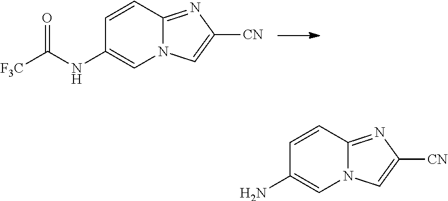 [SMILES: N#Cc1cn2cc(NC(=O)C(F)(F)F)ccc2n1 ; N#Cc1cn2cc(N)ccc2n1]

A solution of N-(2-cyanoimidazo[1,2-a]pyridin-6-yl)-2,2,2-trifluoroacetamide I-179 (150 mg, 0.59 mmol) and K₂CO₃ [584-08-7] (163.1 mg, 1.18 mmol) in DI water (3.11 mL) and MeOH (3.11 mL) was stirred at rt overnight. The reaction mixture was diluted with water (20 mL) and it was extracted with 2-MeTHF, washed with brine, dried on MgSO₄, filtered and concentrated under vacuum to yield I-178 (93 mg, quantitative) as a brown/green solid.

### Synthesis of ethyl 6-iodoimidazo[1,5-a]pyridine-1-carboxylate I-186

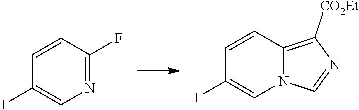 [SMILES: Fc1ccc(I)cn1 ; CCOC(=O)c1ncn2cc(I)ccc12]

Anhydrous DMF (50 mL) was added to a vial charged with NaH [7646-69-7] (60% dispersion in mineral oil, 2.24 g, 56.06 mmol) under nitrogen. The mixture was cooled to 0° C. and ethyl isocyanoacetate [2999-46-4] (6.13 mL, 56.06 mmol) was added dropwise. After 30 min at 0° C., 2-fluoro-5-iodo-pyridine [171197-80-1] (10.0 g, 44.85 mmol) was added in three portions. The reaction was allowed to warm to rt and then heated at 60° C. for 16 hour. The reaction was cooled to rt and diluted with EtOAc (500 mL) and water (300 mL). The organic layer was separated and washed with brine (2×100 mL). The combined aqueous layers were extracted with EtOAc (200 mL). The combined organic layers were dried over MgSO₄, filtered and concentrated in vacuo. The crude product was purified by FCC (Hept/EtOAc 0 to 70%) to obtain I-186 (2.94 g, 21%) as an off-white solid.

### Synthesis of 6-iodoimidazo[1,5-a]pyridine-1-carboxylic acid I-185

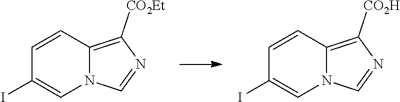 [SMILES: CCOC(=O)c1ncn2cc(I)ccc12 ; O=C(O)c1ncn2cc(I)ccc12]

A 1M aqueous solution of NaOH [1310-73-2] (36 mL, 36 mmol) was added to a solution of ethyl 6-iodoimidazo[1,5-a]pyridine-1-carboxylate I-186 (3.75 g, 11.86 mmol) in THF (35 mL). The reaction mixture was stirred at 60° C. for 2 hours. Volatiles were concentrated under reduced pressure and the aqueous leftovers were treated with 1M aqueous HCl until pH slightly acidic. A solid precipitated and was filtered off, washed with water and then dried at 50° C. under vacuum to yield I-185 (3.25 g, 95%) as an off-white solid.

### Synthesis of 6-iodoimidazo[1,5-a]pyridine-1-carboxamide I-184

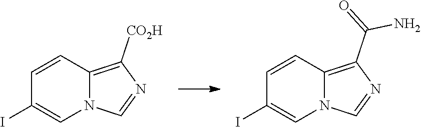 [SMILES: O=C(O)c1ncn2cc(I)ccc12 ; NC(=O)c1ncn2cc(I)ccc12]

SOCl₂ [7719-09-7] (4.1 mL, 56.5 mmol) was added dropwise to a suspension of 6-iodoimidazo[1,5-a]pyridine-1-carboxylic acid I-185 (3.25 g, 11.28 mmol) in dry acetonitrile (30 mL). The reaction was stirred at 60° C. for 1 hour. Volatiles were removed under reduced pressure. The crude product was dissolved in dry DCM (50 mL), the solution cooled to 0° C. and an aqueous solution of NH₃ [7664-41-7] (28% in water, 50 mL, 740 mmol) was added portionwise and the mixture was allowed to warm to rt and stirred at this temperature for 1 hour. The reaction mixture was filtered and the solid cake was washed with water and dried to obtain I-184 (2.29 g, 71%) as a brownish solid.

### Synthesis of 6-iodoimidazo[1,5-a]pyridine-1-carbonitrile I-183

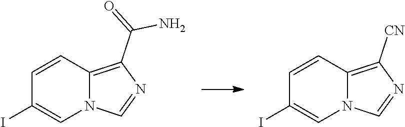 [SMILES: NC(=O)c1ncn2cc(I)ccc12 ; N#Cc1ncn2cc(I)ccc12]

POCl₃ [10025-87-3] (0.82 mL, 8.78 mmol) was added dropwise to a solution of 6-iodoimidazo[1,5-a]pyridine-1-carboxamide I-184 (2.29 g, 7.98 mmol) in anhydrous DMF (23 mL) stirring at 0° C. The reaction was allowed to warm to rt and stirred for 30 min. The reaction was quenched with ice (˜50 mL) and diluted with EtOAc (400 mL) and water (150 mL). The organic layer was separated and the aqueous layer was extracted with EtOAc (100 mL). The combined organic extracts were dried over MgSO₄, filtered and concentrated under reduced pressure to yield desired I-183 (2.15 g, 97%) as a brown solid.

### Synthesis of 6-chloro-2-(2-methoxyethyl)pyridazin-3(2H)-one I-189

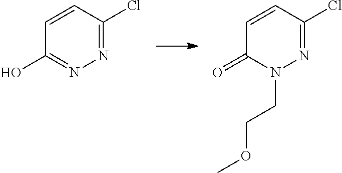

6-Chloropyridazin-3-ol [19064-67-6] (1 g, 7.66 mmol), K₂CO₃ [584-08-7] (1.27 g, 9.18 mmol) and tetrabutylammonium bromide [1643-19-2] (0.049 g, 0.15 mmol) were placed in a 50-mL pressure tube and acetonitrile (12.5 mL) and 2-bromoethyl methyl ether [6482-24-2] (1.08 mL, 1.48 g/mL, 11.49 mmol) were added. The reaction medium was stirred at 115° C. for 5 hours then at rt for 16 hours. It was then poured onto DI water (50 mL) and extracted with DCM (2×). The combined organic sextracts were dried over MgSO₄, filtered and concentrated under vacuum. The obtained crude material was purified by FCC (Hept/EtOAc 0 to 60%) to obtain I-189 (1.13 g, 78%) as a white solid.

### Synthesis of tert-butyl (1-(2-methoxyethyl)-6-oxo-1,6-dihydropyridazin-3-yl)carbamate I-188

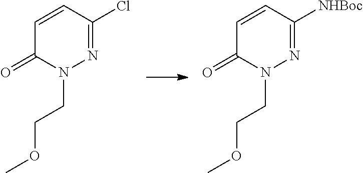 [SMILES: COCCn1nc(Cl)ccc1=O ; COCCn1nc(NC(=O)OC(C)(C)C)ccc1=O]

Two identical reactions were run in parallel wherein bis(dibenzylideneacetone)palladium [32005-36-0] (86.12 mg, 0.15 mmol) was added to a stirred suspension of 6-chloro-2-(2-methoxyethyl)pyridazin-3(2H)-one I-189 (565 mg, 3 mmol), tert-butyl carbamate [4248-19-5] (421 mg, 3.59 mmol), 4,5-bis(diphenylphosphino)-9,9-dimethylxanthene [161265-03-8] (173.3 mg, 0.3 mmol) and cesium carbonate [534-17-8] (2.44 g, 7.49 mmol) in degassed 1,4-dioxane (20 mL) under nitrogen. The mixture was stirred in a sealed tube at 95° C. for two days. The two reaction mixtures were combined and concentrated under vacuum. The crude material was partitioned between DCM and water, the organic layer was separated and the aqueous layer back-extracted with DCM. The combined organic layers were dried over MgSO₄, filtered and evaporated under vacuum. The obtained crude solid was suspended in DCM (20 mL), filtered and the filtrate was concentrated under vacuum and the obtained residue purified by FCC (Hept/EtOAc 100:0 to 0:100) to obtain I-188 (842 mg, 52%) as a yellow solid.

Structural analogues were synthesized according to the above procedure.

| Substrate | Intermediate |
|---|---|
| 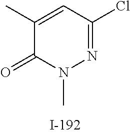 | 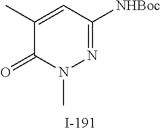 [SMILES: Cc1cc(NC(=O)OC(C)(C)C)nn(C)c1=O] |
| 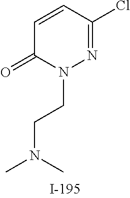 | 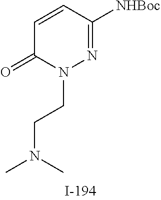 [SMILES: CN(C)CCn1nc(NC(=O)OC(C)(C)C)ccc1=O] |

### Synthesis of 6-amino-2-(2-methoxyethyl)pyridazin-3(2H)-one hydrochloride I-187

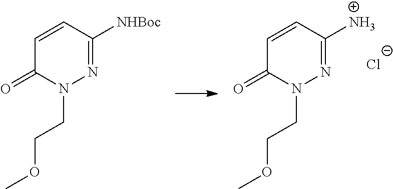 [SMILES: COCCn1nc(NC(=O)OC(C)(C)C)ccc1=O ; COCCn1nc([NH3+])ccc1=O ; [Cl-]]

A 4M HCl solution in 1,4-dioxane [7647-01-0] (14.25 mL, 57 mmol) was added to a solution of tert-butyl (1-(2-methoxyethyl)-6-oxo-1,6-dihydropyridazin-3-yl)carbamate I-188 (842 mg, 3 mmol) in 1,4-dioxane (14.5 mL) and the mixture was stirred at rt for one day The mixture was concentrated in vacuo to yield I-187 (617 mg, 95%) as a dark oil that was used without further purification.

Structural analogues were synthesized according to the above procedure.

| Substrate | Intermediate |
|---|---|
| 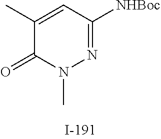 [SMILES: Cc1cc(NC(=O)OC(C)(C)C)nn(C)c1=O] | 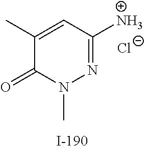 |
| 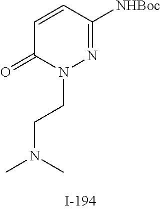 [SMILES: CN(C)CCn1nc(NC(=O)OC(C)(C)C)ccc1=O] | 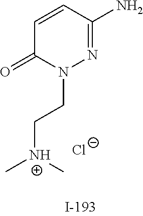 |

### Synthesis of 6-amino-2-(2-methoxyethyl)pyridazin-3(2H)-one hydrochloride I-192

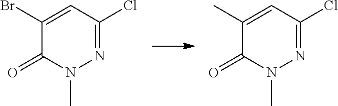

4-Bromo-6-chloro-2-methylpyridazin-3(2H)-one [1178884-53-1] (1.024 g, 4.58 mmol), Cs₂CO₃ [534-17-8] (2.481 g, 7.62 mmol) and 1,1′-bis(diphenylphosphino)ferrocene-palladium(II)dichloroide dichloromethane complex [95464-05-4] (88 mg, 0.108 mmol) were placed in a VLT tube and left under nitrogen (3 vacuum/nitrogen cycles). Trimethylboroxine [823-96-1] (0.5 mL, 3.58 mmol), 1,4-dioxane (17 mL) and DI water (1 mL) were then added. The reaction mixture was stirred at 110° C. for 2.5 hours. The reaction mixture was allowed to cool to rt, diluted with DI water (˜50 mL) and the crude material was extracted with DCM (3×20 mL). The combined organic layers were dried over MgSO₄, filtered and concentrated under reduced pressure. A purification by FCC (Hept/EtOAc 0 to 50%) afforded the I-192 (532 mg, 73%) as a white powder.

### Synthesis of 6-chloro-2-(2-(dimethylamino)ethyl)pyridazin-3(2H)-one I-195

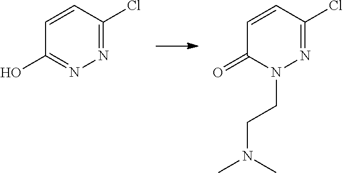

6-Chloropyridazin-3-ol [19064-67-6] (500 mg, 3.83 mmol) was added to a stirring solution of DMEA [108-01-0] (0.46 mL, 0.89 g/mL, 4.6 mmol) in dry toluene (10 mL) under nitrogen. Tsunoda Reagent [157141-27-0] (1.41 mL, 0.92 g/mL, 5.36 mmol) was added and the resulting brown solution was heated at 100° C. for 4 hours. The reaction mixture was allowed to cool to rt and the solvent was evaporated. The residue was purified by FCC (DCM/NH₃ (10% in MeOH) 0 to 5%) to afford I-195 (660 mg, 80%) as a dark brown oil.

### Synthesis of 9-methyl-9H-purin-2-amine I-196

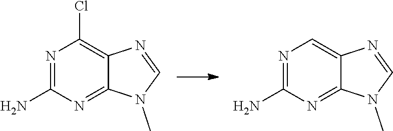

A mixture of 6-chloro-9-methyl-9H-purin-2-amine [3035-73-2] (500 mg, 2.72 mmol), Pd/C (10% wt. Pd, 289.8 mg, 0.27 mmol) in MeOH (30 mL) and THF (50 mL) was hydrogenated at rt overnight. The catalyst was filtered and the filtrate was concentrated to obtain I-196 (780 mg, 85%) as a red solid that was used without further purification.

### Synthesis of 9-methyl-9H-purin-2-amine I-199

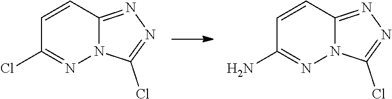 [SMILES: Clc1ccc2nnc(Cl)n2n1 ; Nc1ccc2nnc(Cl)n2n1]

Aqueous ammonia [7664-41-7] (28% in water, 10.53 mL, 0.9 g/mL, 155.76 mmol) was added to a mixture of 3,6-dichloro-[1,2,4]triazolo[4,3-b]pyridazine [33050-38-3] (2 g, 10.58 mmol) in 1,4-dioxane (10.5 mL) was stirred and heated in a pressure tube at 90° C. for 4 hours. The reaction mixture was allowed to cool to rt, the solids were filtered, washed with water and heptane and dried to yield I-199 (1.6 g, yield 89%) as a brown solid.

### Synthesis of 3-oxocyclobutane-1-carboxylate I-1003

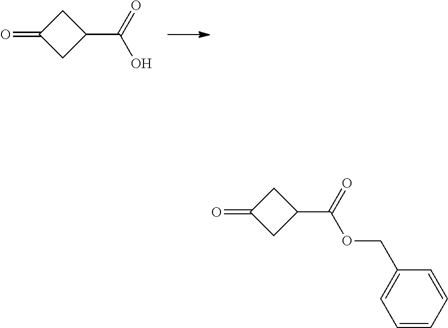 [SMILES: O=C1CC(C(=O)O)C1 ; O=C1CC(C(=O)OCc2ccccc2)C1]

To a mixture of 3-oxocyclobutane-1-carboxylic acid [23761-23-1] (10 g, 87.64 mmol) in DCM (400 ml), Et3N [121-44-8] (18.3 mL, 0.728 g/mL, 131.46 mmol) and DMAP [1122-58-3] (1.07 g, 8.764 mmol) were added at rt. Then the mixture was cooled at 0° C. and benzyl chloroformate [501-53-1] (13.76 mL, 1.195 g/mL, 96.4 mmol) was added dropwise. The mixture was stirred for 24 h at rt. Water was added and the mixture was extracted with DCM, the organic layer was separated. The combined organic layers were dried (Na2SO4), filtered and the solvents evaporated in vacuo. The crude product was purified by flash column chromatography (MeOH in DCM 0/100 to 3/97). The desired fractions were collected, the solvent evaporated in vacuo to yield 3-oxocyclobutane-1-carboxylate I-1003 (9 g, yield 50%).

### Synthesis of benzyl 3-ethyl-3-hydroxycyclobutane-1-carboxylate I-1004

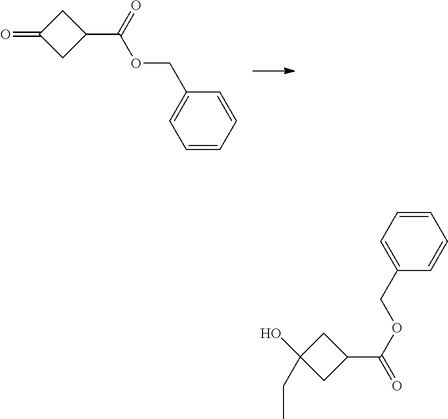 [SMILES: O=C1CC(C(=O)OCc2ccccc2)C1 ; CCC1(O)CC(C(=O)OCc2ccccc2)C1]

To a mixture of benzyl 3-oxocyclobutane-1-carboxylate I-1003 (1500 mg, 7.345 mmol) in THF (20 mL), ethylmagnesium bromide [925-90-6] (3 mL, 3 M, 9 mmol) was added at −60° C. The mixture was stirred at −50° C. for 2 hours. A saturated solution of NH4Cl was added and the crude was extracted with AcOEt (2×10 ml), the combined organic layers were dried and evaporated in vacuo to afford P1. The crude was purified by flash chromatography (silica AcOEt in Hept 0/100 to 20/80), the corresponding layers were evaporated in vacuo to yield benzyl 3-ethyl-3-hydroxycyclobutane-1-carboxylate I-1004 (750 mg, yield 44%) as oil.

1H NMR (400 MHz, CHLOROFORM-d) δ 7.31-7.40 (m, 1H), 5.14 (s, 1H), 2.73 (quin, J=8.38 Hz, 1H), 2.20-2.45 (m, 5H), 1.62 (q, J=7.40 Hz, 2H), 1.28 (br s, 1H), 0.95 (t, J=7.40 Hz, 3H)

### Synthesis of benzyl 3-((tert-butyldimethylsilyl)oxy)-3-ethylcyclobutane-1-carboxylate I-1005

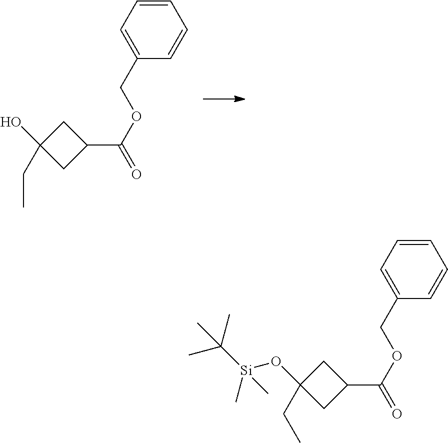 [SMILES: CCC1(O)CC(C(=O)OCc2ccccc2)C1 ; CCC1(O[Si](C)(C)C(C)(C)C)CC(C(=O)OCc2ccccc2)C1]

To a mixture of benzyl 3-ethyl-3-hydroxycyclobutane-1-carboxylate I-1004 (750 mg, 3.2011 mmol) in DCM (25 ml), tert-butyldimethylsilyl trifluoromethanesulphonate [69739-34-0] (1015 mg, 3.84 mmol), DIPEA [7087-68-5], (0.82 mL, 0.75 g/mL, 4.8 mmol) and 4-dimethylaminopyridine [1122-58-3] (40 mg, 0.32 mmol) were added. The mixture was stirred for 24 h at rt. Water was added at rt, and the crude was extracted with DCM (2×10 mL), the combined organic layers were dried and evaporated in vacuo. The crude was purified by flash chromatography (silica AcOEt in Hept 0/100 to 10/90), the corresponding layers were evaporated in vacuo to yield 3-((tert-butyldimethylsilyl)oxy)-3-ethylcyclobutane-1-carboxylate I-1005 (750 mg, yield 67%) as oil.

### Synthesis of benzyl ((1r,3s)-3-((tert-butyldimethylsilyl)oxy)-3-ethylcyclobutyl)carbamate I-1006

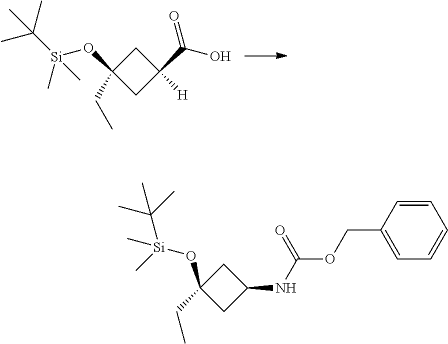 [SMILES: CC[C@]1(O[Si](C)(C)C(C)(C)C)C[C@@H](C(=O)O)C1 ; CC[C@]1(O[Si](C)(C)C(C)(C)C)C[C@@H](NC(=O)OCc2ccccc2)C1]

To a mixture of (1r,3s)-3-((tert-butyldimethylsilyl)oxy)-3-ethylcyclobutane-1-carboxylic acid I-1007 (500 mg, 1.935 mmol) in toluene (20 mL) was added triethylamine [121-44-8] (0.7 mL, 5.036 mmol), followed by diphenylphosphoryl azide [26386-88-9] (800 mg, 2.9 mmol). The reaction mixture was stirred at 80° C. for 3 h. The reaction mixture was then cooled to room temperature and benzyl alcohol [100-51-6](251 mg, 2.3 mmol) was added. The resulting solution was heated to reflux 10 h. The crude was cooled and evaporated in vacuo and treated with a saturated solution of NaHCO₃ and extracted with AcOEt (2×5 ml), the combined organic layers were evaporated to afford an oil. The crude was purified by columm chromatograpy (silica, AcOEt in heptane 0/100 to 20/80), the corresponding fractions were evaporated in vacuo to yield ((1r,3s)-3-((tert-butyldimethylsilyl)oxy)-3-ethylcyclobutyl)carbamate I-1006 (400 mg, yield 57%) as oil which solidified upon standing.

1H NMR (400 MHz, CHLOROFORM-d) δ ppm 0.06 (s, 6H) 0.88 (s, 9H) 0.88-0.93 (m, 3H) 1.48-1.61 (m, 2H) 1.81-1.95 (m, 2H) 2.42-2.61 (m, 2H) 3.65-3.80 (m, 1H) 4.83 (br d, J=5.1 Hz, 1H) 5.08 (s, 2H) 7.28-7.43 (m, 5H)

### Synthesis of (1r,3s)-3-((tert-butyldimethylsilyl)oxy)-3-ethylcyclobutan-1-amine I-1008

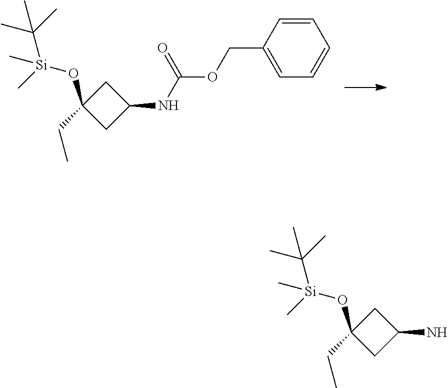 [SMILES: CC[C@]1(O[Si](C)(C)C(C)(C)C)C[C@@H](NC(=O)OCc2ccccc2)C1 ; CC[C@]1(O[Si](C)(C)C(C)(C)C)C[C@@H](N)C1]

To a mixture of benzyl ((1r,3s)-3-((tert-butyldimethylsilyl)oxy)-3-ethylcyclobutyl)carbamate I-1006 (400 mg, 1.1 mmol) in THF (40 mL), Pd/C (10%) (120 mg, 0.112 mmol) was added under N2 atmosphere, the mixture was hydrogenated with balloon at rt for 16 h. The crude was filtered over celite and evaporated in vacuo. The crude product was purified by flash column chromatography (silica, MeOH in DCM 0/100 to 10/90), the corresponding fractions were evaporated in vacuo to yield (1r,3s)-3-((tert-butyldimethylsilyl)oxy)-3-ethylcyclobutan-1-amine I-1008 (200 mg, yield 79%) as transparent oil.

1H NMR (400 MHz, CHLOROFORM-d) δ 2.86-2.99 (m, 1H), 2.39-2.51 (m, 2H), 1.69-1.82 (m, 2H), 1.50 (q, J=7.24 Hz, 4H), 0.81-0.93 (m, 12H), 0.05-0.12 (m, 6H)

Structure analogs were synthesized using the same procedure.

| Starting material | Intermediate |
|---|---|
| 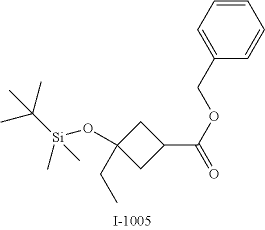 [SMILES: CCC1(O[Si](C)(C)C(C)(C)C)CC(C(=O)OCc2ccccc2)C1] | 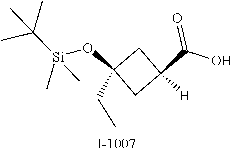 [SMILES: CC[C@]1(O[Si](C)(C)C(C)(C)C)C[C@@H](C(=O)O)C1] |

### Synthesis of 2-(benzyloxy)acetyl chloride I-1009

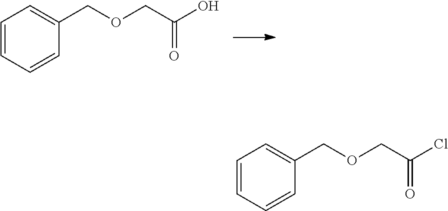 [SMILES: O=C(O)COCc1ccccc1 ; O=C(Cl)COCc1ccccc1]

Thionyl chloride [7719-09-7] (588 μL, 7.82 mmol) was added to a stirred solution of benzyloxyacetic acid [30379-55-6] (1 g, 6.02 mmol) in DCM anhydrous (20 mL) at 0° C. The mixture was stirred from 45° C. to rt for 3 h. The solvent was evaporated in vacuo to yield 2-(benzyloxy)acetyl chloride I-1009 (1.12 g, quantitative) as a beige solid.

### Synthesis of 1-fluorocyclopropane-1-carbonyl chloride I-111

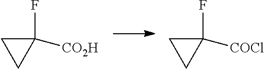 [SMILES: O=C(O)C1(F)CC1 ; O=C(Cl)C1(F)CC1]

Oxalyl chloride [79-37-8] (3.08 mL, 1.48 g/mL, 35.9 mmol) was added dropwise to a solution of 1-fluorocyclopropanecarboxylic acid [137081-41-5] (4.14 g, 39.8 mmol) and DMF (100 μL, 0.94 g/mL, 1.29 mmol) in dry DCM (150 mL). The reaction mixture was stirred at rt for 16 hours and then evaporated under vacuum (36° C., 400 mbars) and the obtained crude acyl chloride I-111 used without further purification. The crude was used without further purification in the next step.

### Synthesis of (tributylstannyl)methanol I-117

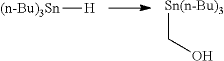

A 2.5M solution of n-BuLi in hexanes [109-72-8] (5.4 mL, 13.5 mmol) was added dropwise to a stirred solution of diisipropylamine [108-18-9] (2 mL, 0.72 g/mL, 14.26 mmol) in dry THF (45 mL) at −20° C. The reaction mixture was stirred at −20° C. for 30 min. Then was cooled to −78° C. and tributyltin hydride [688-73-3] (3.53 mL, 1.08 g/mL, 12.73 mmol) was added dropwise. The mixture was allowed to warm to 0° C. for 30 min. Then was cooled to −78° C. and paraformaldehyde [30525-89-4] (463 mg, 5.09 mmol) was added portionwise. After addition, the reaction was allowed to warm slowly from −78° C. to rt over 30 min and stirred at rt for further 30 min. The mixture was diluted with water and extracted with Et₂O. The organic layer was separated, dried (MgSO₄), filtered and the volatiles evaporated in vacuo. The crude product was purified by FCC (Hept/EtOAc 0 to 10%) to yield I-117 (2.8 g, 68%) as a colorless oil.

### Synthesis of methyl 4-(1-fluorocyclopropyl)benzoate I-1010

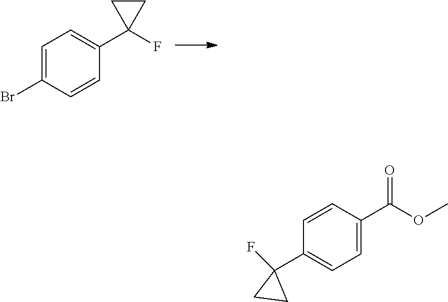

A mixture of MeOH (10 mL) and THF, dry (30 mL) was degassed with nitrogen. Triethylamine [121-44-8] (6.48 mL, 46.5 mmol), 4,5-bis(diphenylphosphino)-9,9-dimethylxanthene [161265-03-8] (430.48 mg, 0.74 mmol), 1-bromo-4-(1-fluorocyclopropyl)benzene [1783975-92-7] (4 g, 18.6 mmol) and palladium(II) acetate [3375-31-3] (83.51 mg, 0.37 mmol) were added. Then the mixture was stirred at 120° C. under 20 bar carbon monoxide in an autoclave for 24 h. The solvent was evaporated, taken up in sat NaHCO₃ solution, extracted with DCM, dried on MgSO4 and evaporated. The residue was purified on a column with silicagel, eluent. EtOAc in Heptane, from 0 to 10%. The pure fractions were evaporated, yielding methyl 4-(1-fluorocyclopropyl)benzoate I-1010 (2.92 g, yield 81%) as colorless oil which solidified to a white solid upon cooling to rt.

### Synthesis of 3-fluoro-4-(trifluoromethyl)benzoic acid I-93

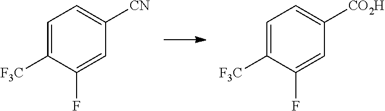 [SMILES: N#Cc1ccc(C(F)(F)F)c(F)c1 ; O=C(O)c1ccc(C(F)(F)F)c(F)c1]

To a mixture of 3-fluoro-4-(trifluoromethyl)benzonitrile [231953-38-1] (1 g, 5.29 mmol) in EtOH (20 ml) was added a 2M aqueous solution NaOH [1310-73-2] (4 mL, 8 mmol). The mixture was heated for at 80° C. for 16 hours. The mixture was cooled at rt and aqueous HCl (2M) was added until pH=2. The mixture was extracted with EtOAc (2×5 ml), the combined organic layers were dried over MgSO4 and evaporated in vacuo to afford I-93 (0.9 g, 82%) as a white solid.

### Synthesis of 2-fluoro-6-iodo-4-(trifluoromethyl)benzoic acid I-97

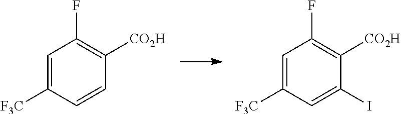 [SMILES: O=C(O)c1ccc(C(F)(F)F)cc1F ; O=C(O)c1c(F)cc(C(F)(F)F)cc1I]

Iodine [7553-56-2] (2.68 g, 10.6 mmol) was added to a stirred solution of 2-fluoro-4-(trifluoromethyl)benzoic acid [115029-24-8] (2 g, 9.61 mmol)), PIDA [3240-34-4](3.4 g, 10.6 mmol) and Pd(OAc)₂ [3375-31-3] (107.8 mg, 0.48 mmol) in DMF (30 mL) under nitrogen. The mixture was stirred at 100° C. for 16 hours. EtOAc and 1M aqueous HCl were added, the organic layer was separated, dried over MgSO₄, filtered and the volatiles were removed in vacuo to yield crude I-97 as a brown sticky solid that was used without further purification.

The crude was used in the next reaction step without further purification.

### Synthesis of tert-butyl 3-fluoro-4-(trifluoromethyl)benzoate I-94

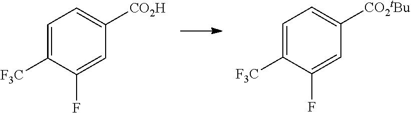 [SMILES: O=C(O)c1ccc(C(F)(F)F)c(F)c1 ; CC(C)(C)OC(=O)c1ccc(C(F)(F)F)c(F)c1]

2-Methyl-2-propanol [75-65-0] (4.6 g, 62.5 mmol), N,N-dicyclohexylcarbodiimide [538-75-0] (13.9 g, 67.3 mmol) and 4-(dimethylamino)pyridine [1122-58-3] (0.587 g, 4.8 mmol) were added to a mixture of 3-fluoro-4-(trifluoromethyl)benzoic acid I-93 (10 g, 48 mmol) in THF (250 mL). The mixture was stirred at rt for 16 hours. The crude mixture was filtered and washed with cold THF. The solid was discarded and the filtrated was evaporated in vacuo. The residue was purified by FCC (DCM) to afford I-94 (9 g, 71%).

### Synthesis of methyl 4-bromo-2-iodobenzoate I-1011

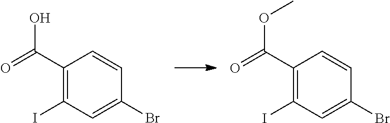

Cesium carbonate [534-17-8] (5.98 g, 18.35 mmol) and methyl iodide [74-88-4] (1.14 mL, 2.28 g/mL, 18.35 mmol) were added to a stirred solution of 4-bromo-2-iodobenzoic acid [1133123-02-0] (5 g, 15.29 mmol) in DMF (9 mL) at rt for 16 h. The reaction mixture was diluted with EtOAc and filtered through a filter paper. The organic layer was concentrated in vacuo. The crude was purified by flash column chromatography (silica; EtOAc in Heptane from 0/100 to 20/80). The desired fractions were collected and concentrated in vacuo to yield methyl 4-bromo-2-iodobenzoate I-1011 (4.27 g, 80%) as a colorless oil.

Structural analogues were synthesized according to the above procedure.

| Reagent | Intermediate |
|---|---|
| 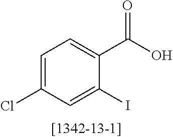 [SMILES: O=C(O)c1ccc(Cl)cc1I] | 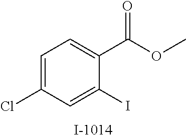 |
| 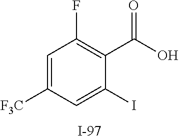 | 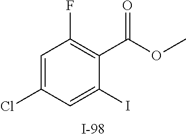 |

### Synthesis of tert-butyl 3-fluoro-2-isobutyryl-4-(trifluoromethyl)benzoate I-95

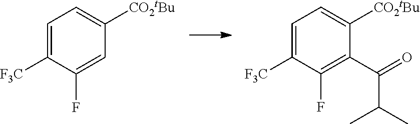 [SMILES: CC(C)(C)OC(=O)c1ccc(C(F)(F)F)c(F)c1 ; CC(C)C(=O)c1c(C(=O)OC(C)(C)C)ccc(C(F)(F)F)c1F]

A 2M solution of LDA (in THF/n-heptane) [4111-54-0] (18.9 mL, 2 M, 37.8 mmol) was added to a mixture of tert-butyl 3-fluoro-4-(trifluoromethyl)benzoate I-94 (5 g, 18.9 mmol) in dry THF (125 ml) at −78° C. The mixture was stirred at −78° C. for 45 min, then isobutyryl chloride [79-30-1] (2.1 mL, 1.017 g/mL, 20.8 mmol) was added dropwise at −78° C. The reaction was stirred at −78° C. for 2 hours. The reaction was quenched by addition of a saturated aqueous solution of NH₄Cl was added at −78° C. The crude mixture was allowed to warm to rt and extracted with EtOAc (2×25 ml), the organic layer was separated dried and evaporated in vacuo. The residue was purified by FCC (DCM) yielding I-95 (5.2 g, 82%) as an oil.

### Synthesis of methyl 2-(2-(benzyloxy)acetyl)-4-bromobenzoate I-1012

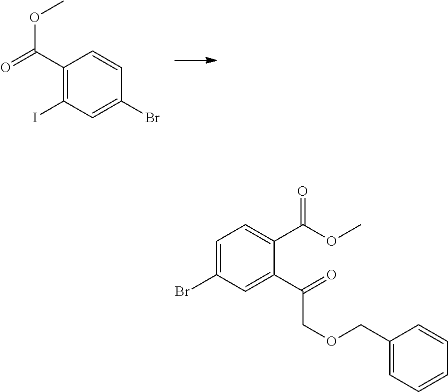 [SMILES: COC(=O)c1ccc(Br)cc1I ; COC(=O)c1ccc(Br)cc1C(=O)COCc1ccccc1]

Isopropylmagnesium chloride solution (2 M in THF) [1068-55-9] (807 μL, 1.61 mmol) was added dropwise to a solution of methyl 4-bromo-2-iodobenzoate I-1011 (500 mg, 1.47 mmol) in THF anhydrous (7.5 mL) at −78° C. and the resulting mixture was stirred at 0° C. for 30 min under nitrogen atmosphere. Then zinc bromide anhydrous [7699-45-8] (367 mg, 1.61 mmol) was added and the reaction mixture was stirred at 0° C. for 15 min. 2-(Benzyloxy)acetyl chloride I-1009 (325 mg, 1.76 mmol) and tetrakis(triphenylphosphine)palladium (0) [14221-01-3] (86 mg, 0.073 mmol) were added and the resulting mixture was stirred at 60° C. for 2 h under nitrogen atmosphere. The reaction mixture was diluted with sat. aqueous NH4Cl and extracted with EtOAc. The organic layer was separated, dried (MgSO4), filtered and the solvents evaporated in vacuo. The crude product was purified by flash column chromatography (silica; DCM in heptane 0/100 to 100/0). The desired fractions were collected and concentrated in vacuo to yield methyl 2-(2-(benzyloxy)acetyl)-4-bromobenzoate I-1012 (468 mg, 82%) as a yellow sticky solid.

Structural analogues were synthesized according to the above procedure.

| Reagent | Intermediate | product |
|---|---|---|
| 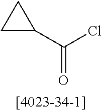 | 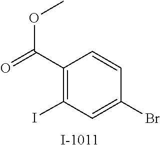 | 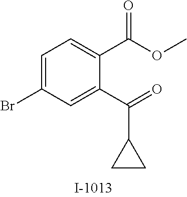 [SMILES: COC(=O)c1ccc(Br)cc1C(=O)C1CC1] |
| 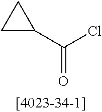 | 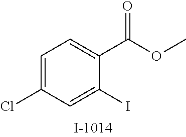 | 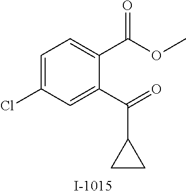 [SMILES: COC(=O)c1ccc(Cl)cc1C(=O)C1CC1] |
| 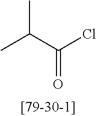 | 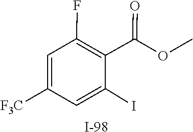 | 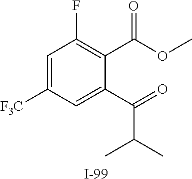 |
| 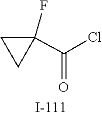 | 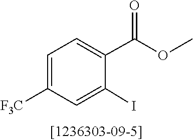 | 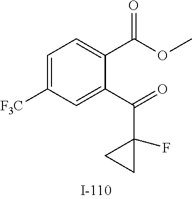 [SMILES: COC(=O)c1ccc(C(F)(F)F)cc1C(=O)C1(F)CC1] |

### Synthesis of 5-(trifluoromethoxy)isobenzofuran-1(3H)-one I-1016

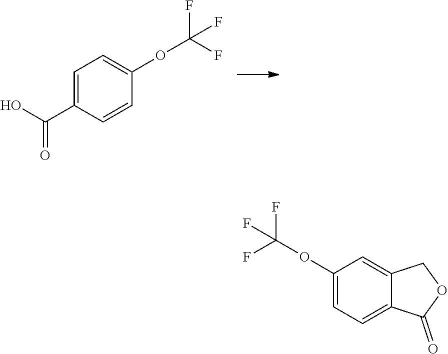 [SMILES: O=C(O)c1ccc(OC(F)(F)F)cc1 ; O=C1OCc2cc(OC(F)(F)F)ccc21]

4-(Trifluoromethoxy)benzoic acid [330-12-1] (2000 mg, 9.7 mmol) and dibromomethane (37 mL) were added to potassium phosphate dibasic [7758-11-4](5070 mg, 29.11 mmol) and Pd(OAc)_{2 [3375}-31-3] (218 mg, 0.97 mmol) in a sealed tube under nitrogen. The mixture was stirred at 140° C. for 24 h. The reaction mixture was filtered through a short pad of celite, and the solvent was removed in vacuo. The crude product was purified by flash column chromatography (silica; EtOAc in heptane 0/100 to 20/80). The desired fractions were collected and concentrated in vacuo to yield 5-(trifluoromethoxy)isobenzofuran-1(3H)-one I-1016 (771 mg, 36%) as a yellowish solid.

Structural analogs were synthesized using the same procedure

| Intermediate | Product |
|---|---|
| 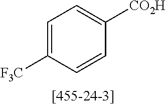 [SMILES: O=C(O)c1ccc(C(F)(F)F)cc1] | 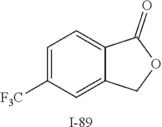 |
| 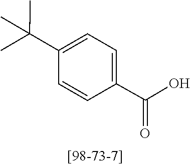 | 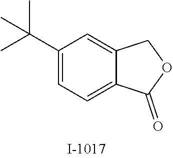 |
| 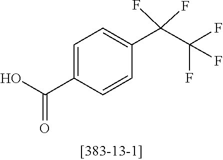 [SMILES: O=C(O)c1ccc(C(F)(F)C(F)(F)F)cc1] | 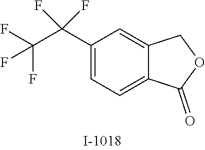 [SMILES: O=C1OCc2cc(C(F)(F)C(F)(F)F)ccc21] |
| 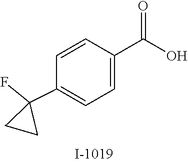 [SMILES: O=C(O)c1ccc(C2(F)CC2)cc1] | 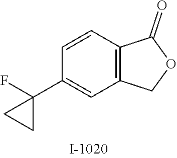 [SMILES: O=C1OCc2cc(C3(F)CC3)ccc21] |

### Synthesis of 3,5-dibromoisobenzofuran-1(3H)-one I-1021

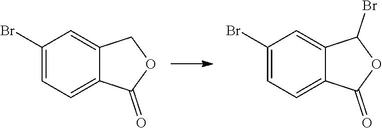 [SMILES: O=C1OCc2cc(Br)ccc21 ; O=C1OC(Br)c2cc(Br)ccc21]

N-Bromosuccinimide [128-08-5] (10 g, 56.33 mmol) and azobisisobutyronitrile (AIBN) [78-67-1] (231 mg, 1.41 mmol) were added to a stirred solution of 5-bromophthalide [64169-34-2] (10 g, 46.94 mmol) in DCE (200 mL). The mixture was stirred at 80° C. for 18 h. The reaction mixture was cooled at rt and the solvent was concentrated. The crude was purified by flash column chromatography (silica; EtOAc in heptane 0/100 to 20/80). The desired fractions were collected and concentrated in vacuo to yield 3,5-dibromoisobenzofuran-1(3H)-one I-1021 (10.9 g, 73%) as a white solid.

Structural analogs were synthesized using the same procedure.

| Intermediate | Product |
|---|---|
| 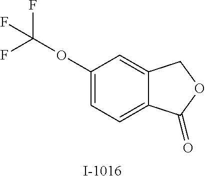 [SMILES: O=C1OCc2cc(OC(F)(F)F)ccc21] | 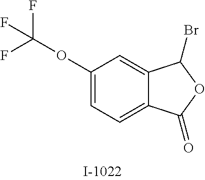 [SMILES: O=C1OC(Br)c2cc(OC(F)(F)F)ccc21] |
| 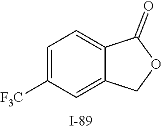 | 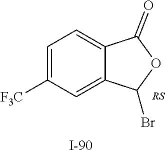 |
| 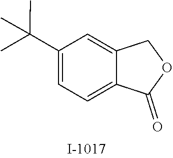 | 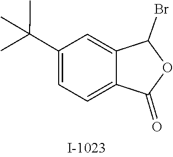 |
| 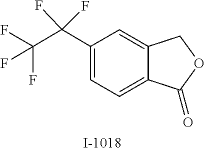 [SMILES: O=C1OCc2cc(C(F)(F)C(F)(F)F)ccc21] | 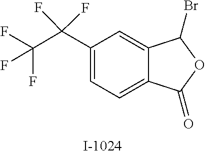 [SMILES: O=C1OC(Br)c2cc(C(F)(F)C(F)(F)F)ccc21] |
| 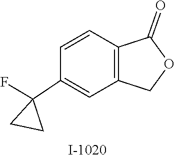 [SMILES: O=C1OCc2cc(C3(F)CC3)ccc21] | 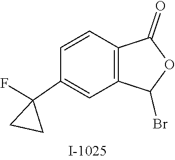 [SMILES: O=C1OC(Br)c2cc(C3(F)CC3)ccc21] |

### Synthesis of 5-bromo-3-hydroxyisobenzofuran-1(3H)-one I-1026

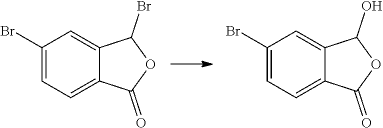 [SMILES: O=C1OC(Br)c2cc(Br)ccc21 ; O=C1OC(O)c2cc(Br)ccc21]

3,5-Dibromoisobenzofuran-1(3H)-one I-1021 (7.34 g, 25.14 mmol) was suspended in water (300 mL) and heated at 100° C. for 1 h. The mixture was cooled at rt and extracted with DCM:MeOH 9:1 three times. The combined organic layers were dried over MgSO4 anh, filtered and concentrated under reduced pressure to yield 5-bromo-3-hydroxyisobenzofuran-1(3H)-one I-1026 (5.9 g, 99%) as a white solid.

Structural analogs were synthesized using the same procedure.

| Intermediate | Product |
|---|---|
| 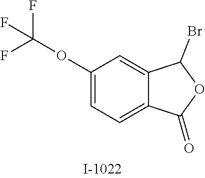 [SMILES: O=C1OC(Br)c2cc(OC(F)(F)F)ccc21] | 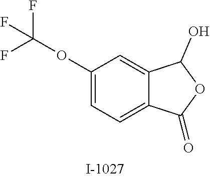 [SMILES: O=C1OC(O)c2cc(OC(F)(F)F)ccc21] |
| 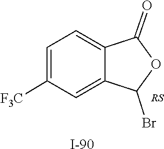 | 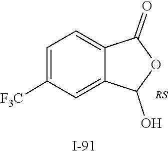 |
| 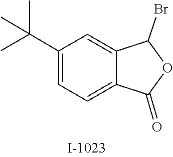 | 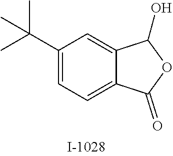 |
| 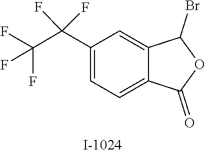 [SMILES: O=C1OC(Br)c2cc(C(F)(F)C(F)(F)F)ccc21] | 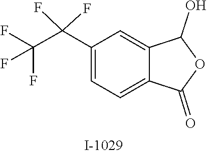 [SMILES: O=C1OC(O)c2cc(C(F)(F)C(F)(F)F)ccc21] |
| 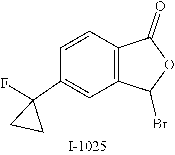 [SMILES: O=C1OC(Br)c2cc(C3(F)CC3)ccc21] | 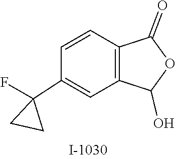 [SMILES: O=C1OC(O)c2cc(C3(F)CC3)ccc21] |

### Synthesis of 2-(4-methoxyphenyl)-4,4-dimethyl-5H-oxazole (I-1)

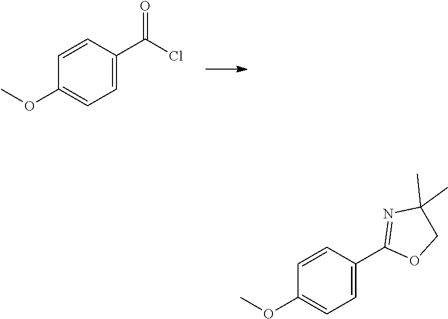

A solution of 2-amino-2-methyl-1-propanol [124-68-5] (53.03 g, 0.59 mol) in anhydrous DCM (200 mL) was dropwise added in 30 min to a stirred solution of ethyl 4-methoxybenzoyl chloride [100-07-2] (51.18 g, 0.3 mol) in anhydrous DCM (254 mL) under nitrogen keeping the temperature at about 18° C. using an ice/water bath. After 3 h under stirring the precipitate was filtered through celite and washed with DCM. The organic phase was stirred under nitrogen at 2° C. and dropwise added to thionyl chloride [7719-09-7] (65.29 ml, 0.9 mol), keeping the temperature below 10° C. At the end of the dropping the reaction mixture was stirred at room temperature for 18 h, then concentrated under vacuo. The residue was purified by column chromatography (silica, MeOH in DCM 0/100 to 1/99). The desired fractions were collected and the solvents evaporated in vacuo to yield 2-(4-methoxyphenyl)-4,4-dimethyl-5H-oxazole (I-1) (42 g, 68%) as yellow oil.

### Synthesis of [2-(4,4-dimethyl-5H-oxazol-2-yl)-5-methoxy-phenyl]-2-methyl-propan-1-ol (I-2)

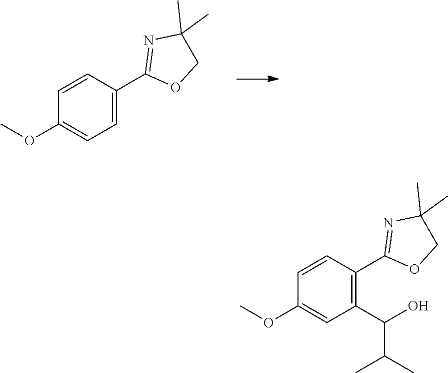

A 1M solution of 2,2,6,6-Tetramethylpiperidinylmagnesium chloride lithium chloride complex in THF/toluene [898838-07-8] (60 ml, 60 mmol) was added dropwise to a stirred solution of 2-(4-methoxyphenyl)-4,4-dimethyl-5H-oxazole (I-1) (5.29 g, 25.78 mmol) in anhydrous THF (100 mL) at room temperature. After 4 h stirring, the reaction mixture was cooled to 0° C. and a solution of isobutyraldehyde [78-84-2] (7 mL, 76.69 mmol) in anhydrous THF (10 mL) was added dropwise. The resulting reaction mixture was stirred at room temperature for 2 h. The solvent was partially removed in vacuo and the residue was diluted with saturated aqueous solution of NH₄Cl and extracted with EtOAc. The combined organic layers were separated, dried (MgSO4), filtered and the solvents evaporated in vacuo. The crude product was purified by flash column chromatography (silica, EtOAc in heptane 0/100 to 50/50). The desired fractions were collected and concentrated in vacuo to yield [2-(4,4-dimethyl-5H-oxazol-2-yl)-5-methoxy-phenyl]-2-methyl-propan-1-ol (I-2) (6.3 g, 89%) as yellow oil.

Structural analogs were synthesized using the same procedure.

| Reagent | Starting material | Intermediate |
|---|---|---|
| 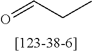 | 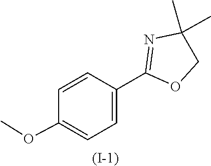 | 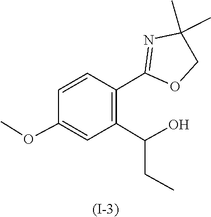 |

### Synthesis of 1-[2-(4,4-dimethyl-5H-oxazol-2-yl)-5-methoxy-phenyl]-2-methyl-propan-1-one (I-4)

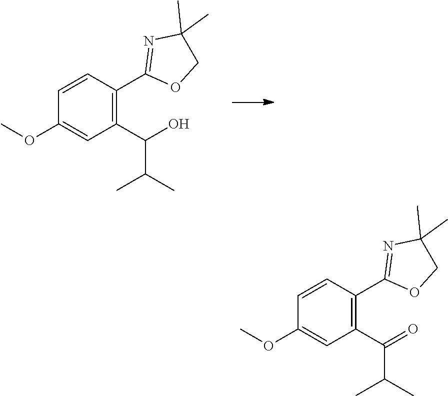

Dess-Martin periodinane [87413-09-0] (4.07 g, 9.59 mmol) was added to a stirred solution of [2-(4,4-dimethyl-5H-oxazol-2-yl)-5-methoxy-phenyl]-2-methyl-propan-1-ol (I-2) (1.98 g, 7.14 mmol) in anhydrous DCM (120 mL) at room temperature. After 2.5 h stirring, additional Dess-Martin periodinane [87413-09-0] (1.35 g, 3.18 mmol) was added and the resulting mixture was stirred for 1 h more. The mixture was filtered and to the filtrate was added 20% aqueous solution of Na₂S₂O₃. The product was extracted with DCM and the organic layer was further washed with saturated aqueous solution of NaHCO₃ and brine. The organic layer was dried (MgSO4), filtered and the solvents evaporated in vacuo to yield 1-[2-(4,4-dimethyl-5H-oxazol-2-yl)-5-methoxy-phenyl]-2-methyl-propan-1-one (I-4) (0.50 g, 48% purity) as yellow solid, that was used in next step without further purification.

Structural analogs were synthesized using the same procedure.

| Starting material | Intermediate |
|---|---|
| 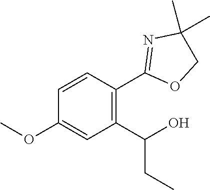 | 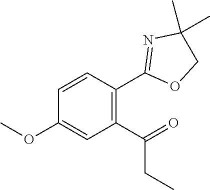 |
| (I-3) | (I-5) |
| 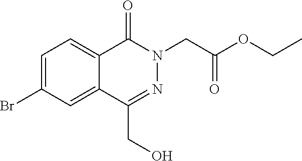 [SMILES: CCOC(=O)Cn1nc(CO)c2cc(Br)ccc2c1=O] | 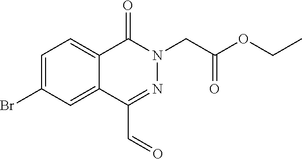 [SMILES: CCOC(=O)Cn1nc(C=O)c2cc(Br)ccc2c1=O] |
| (I-1031) | (I-1032) |

### Synthesis of ethyl 4-methoxy-2-propanoyl-benzoate (I-6)

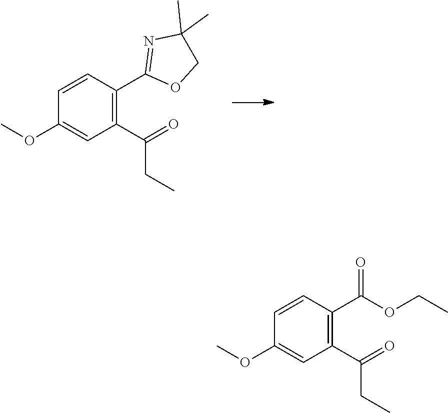

H₂SO₄ [7664-93-9] (2.5 mL, 46.9 mmol) was added dropwise to a stirred solution of 1-[2-(4,4-dimethyl-5H-oxazol-2-yl)-5-methoxy-phenyl]propan-1-one (I-5) (2 g, 7.65 mmol) in a mixture of water (3 mL) and EtOH (57 mL). The mixture was stirred at 90° C. for 20 h. The solvent was concentrated in vacuo and the residue was diluted with water and extracted with Et₂O. The organic layer was separated, dried (MgSO4), filtered and the solvents evaporated in vacuo to yield ethyl 4-methoxy-2-propanoyl-benzoate (I-6) (1.52 g, 84%), that was used in next step without further purification.

### Synthesis of 5-methyl-2-phenyl-isoindoline-1,3-dione (I-7)

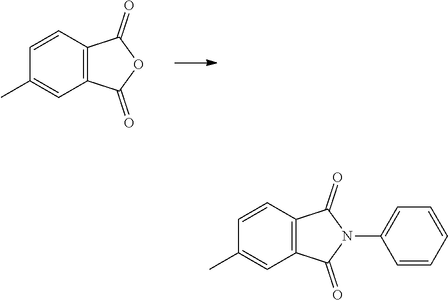 [SMILES: Cc1ccc2c(c1)C(=O)OC2=O ; Cc1ccc2c(c1)C(=O)N(c1ccccc1)C2=O]

Aniline [62-53-3] (1.24 mL, 13.57 mmol) was added dropwise to a stirred solution of 5-methylisobenzofuran-1,3-dione [19438-61-0] (2 g, 12.33 mmol) in AcOH [64-19-7] (12.3 mL). The mixture was stirred at 140° C. for 2 h. To the cooled mixture was added water and the resulting reaction mixture was stirred at room temperature for 2 h. The solid formed was filtered and washed with additional water to yield 5-methyl-2-phenyl-isoindoline-1,3-dione (I-7) (2.77 g, 95%) as a white solid, that was used in next step without further purification.

Structural analogs were synthesized using the same procedure.

| Starting material | Intermediate |
|---|---|
| 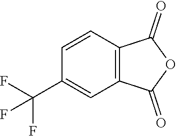 [SMILES: O=C1OC(=O)c2cc(C(F)(F)F)ccc21] | 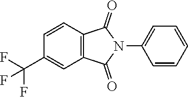 [SMILES: O=C1c2ccc(C(F)(F)F)cc2C(=O)N1c1ccccc1] |
| [26238-14-2] | (I-8) |
| 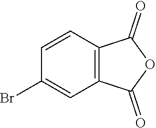 [SMILES: O=C1OC(=O)c2cc(Br)ccc21] | 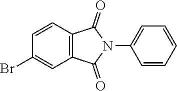 [SMILES: O=C1c2ccc(Br)cc2C(=O)N1c1ccccc1] |
| [86-9-8] | (I-17) |
| 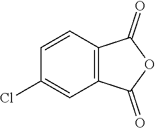 [SMILES: O=C1OC(=O)c2cc(Cl)ccc21] | 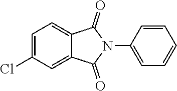 [SMILES: O=C1c2ccc(Cl)cc2C(=O)N1c1ccccc1] |
| [118-45-6] | (I-1033) |

### Synthesis of 5-bromo-3-ethyl-3-hydroxy-2-phenyl-isoindolin-1-one (I-9)

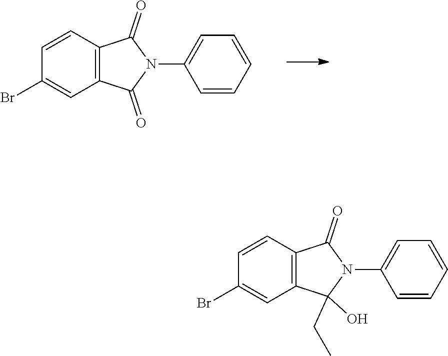 [SMILES: O=C1c2ccc(Br)cc2C(=O)N1c1ccccc1 ; CCC1(O)c2cc(Br)ccc2C(=O)N1c1ccccc1]

A 3M solution of ethylmagnesium bromide [925-90-6] (1.65 mL, 4.96 mmol) was added dropwise to a stirred solution of 5-bromo-2-phenyl-isoindoline-1,3-dione [82104-66-3] (1 g, 3.31 mmol) in THF (20 mL) at 0° C. and under nitrogen. After 5 min stirring, the mixture was quenched by the addition of water. The solvents were evaporated in vacuo to yield 5-bromo-3-ethyl-3-hydroxy-2-phenyl-isoindolin-1-one (I-9) (1.1 g, quant.) as yellowish oil, that was used in next step without further purification.

Structural analogs were synthesized using the same procedure.

| Reagent | Starting material | Intermediate |
|---|---|---|
| 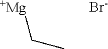 [925-90-6] | 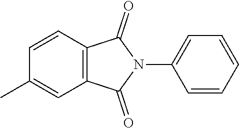 [SMILES: Cc1ccc2c(c1)C(=O)N(c1ccccc1)C2=O] (I-7) | 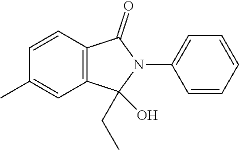 [SMILES: CCC1(O)c2cc(C)ccc2C(=O)N1c1ccccc1] (I-10) |
| 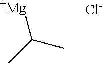 [1068-55-9] | 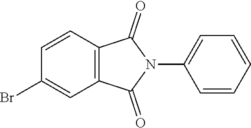 [SMILES: O=C1c2ccc(Br)cc2C(=O)N1c1ccccc1] (I-17) | 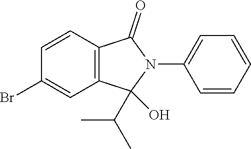 [SMILES: CC(C)C1(O)c2cc(Br)ccc2C(=O)N1c1ccccc1] (I-19) |
| 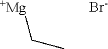 [925-90-6] | 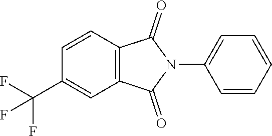 [SMILES: O=C1c2ccc(C(F)(F)F)cc2C(=O)N1c1ccccc1] (I-8) | 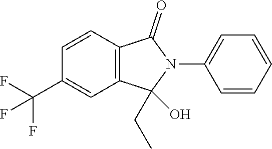 [SMILES: CCC1(O)c2cc(C(F)(F)F)ccc2C(=O)N1c1ccccc1] (I-11) |
| 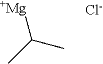 [1068-55-9] | 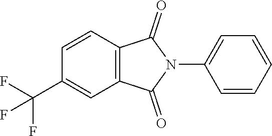 [SMILES: O=C1c2ccc(C(F)(F)F)cc2C(=O)N1c1ccccc1] (I-8) | 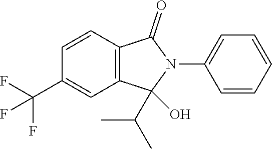 [SMILES: CC(C)C1(O)c2cc(C(F)(F)F)ccc2C(=O)N1c1ccccc1] (I-20) |
| 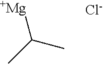 [1068-55-9] | 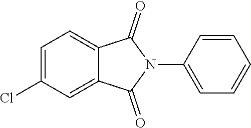 [SMILES: O=C1c2ccc(Cl)cc2C(=O)N1c1ccccc1] (I-1033) | 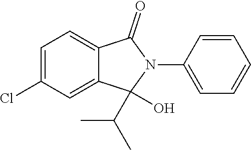 [SMILES: CC(C)C1(O)c2cc(Cl)ccc2C(=O)N1c1ccccc1] (I-1034) |

### Synthesis of 4-isopropyl-6-methoxy-2H-phthalazin-1-one (I-12)

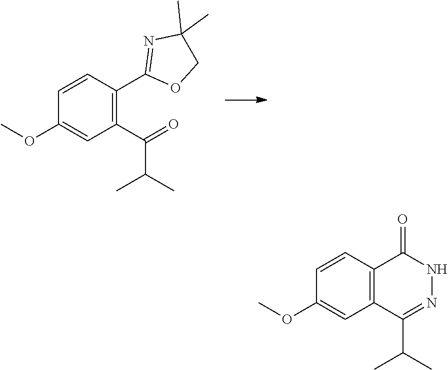

Hydrazine hydrate [7803-57-8] (0.15 mL, 3.09 mmol) was added to a stirred solution of 1-[2-(4,4-dimethyl-5H-oxazol-2-yl)-5-methoxy-phenyl]-2-methyl-propan-1-one (I-4) (0.50 g, 48% purity) in AcOH [64-19-7] (3 mL). The mixture was stirred at 80° C. for 18 h. The mixture was diluted with water and extracted with DCM. The aqueous phase was basified with aqueous saturated solution of NaHCO₃ and extracted with DCM. The combined organic layers were dried (MgSO4), filtered and the solvents evaporated in vacuo. The crude product was purified by flash column chromatography (silica, EtOAc in heptane 0/100 to 50/50). The desired fractions were collected and the solvents concentrated in vacuo to yield 4-isopropyl-6-methoxy-2H-phthalazin-1-one (I-12) (103 mg, 54%).

Structural analogs were synthesized using the same procedure.

| Starting material | Intermediate |
|---|---|
| 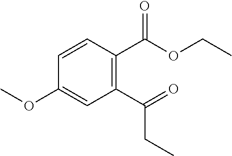 [SMILES: CCOC(=O)c1ccc(OC)cc1C(=O)CC] | 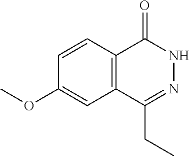 |
| (I-5) | (I-13) |

### Synthesis of 6-bromo-4-ethyl-2H-phthalazin-1-one (I-14)

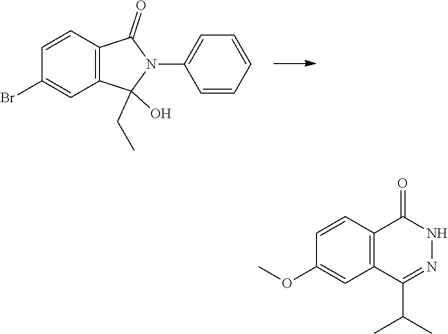 [SMILES: CCC1(O)c2cc(Br)ccc2C(=O)N1c1ccccc1 ; COc1ccc2c(=O)[nH]nc(C(C)C)c2c1]

Hydrazine hydrate [7803-57-8] (0.52 mL, 16.56 mmol) was added to a stirred solution of 5-bromo-3-ethyl-3-hydroxy-2-phenyl-isoindolin-1-one (I-9) (1.1 g, 3.31 mmol) in EtOH (12 mL) in a sealed tube. The mixture was stirred at 80° C. for 16 h. Additional hydrazine hydrate [7803-57-8] (0.52 mL, 16.56 mmol) was added and the mixture was stirred at 80° C. for 5 h. Additional hydrazine hydrate [7803-57-8] (1.03 mL, 33.11 mmol) was added and the mixture was stirred at 80° C. for 3 days. After cooling the mixture, the solid formed was filtered and dried under vacuo to yield 6-bromo-4-ethyl-2H-phthalazin-1-one (1-14) (500 mg, 60%).

Structural analogs were synthesized using the same procedure.

| Starting material | Intermediate |
|---|---|
| 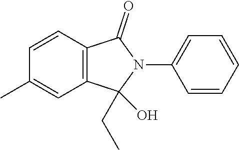 [SMILES: CCC1(O)c2cc(C)ccc2C(=O)N1c1ccccc1] | 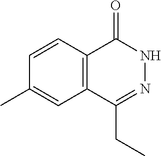 |
| (I-10) | (I-16) |
| 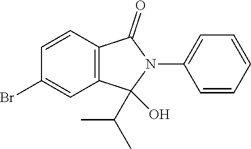 [SMILES: CC(C)C1(O)c2cc(Br)ccc2C(=O)N1c1ccccc1] | 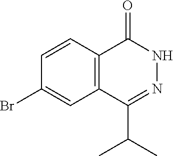 |
| (I-27) | (I-15) |
| 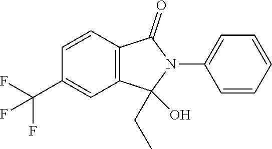 [SMILES: CCC1(O)c2cc(C(F)(F)F)ccc2C(=O)N1c1ccccc1] | 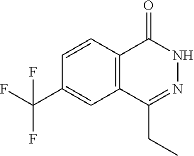 [SMILES: CCc1n[nH]c(=O)c2ccc(C(F)(F)F)cc12] |
| (I-11) | (I-18) |
| 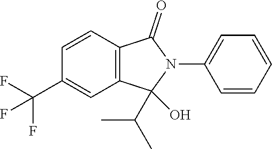 [SMILES: CC(C)C1(O)c2cc(C(F)(F)F)ccc2C(=O)N1c1ccccc1] | 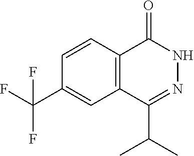 [SMILES: CC(C)c1n[nH]c(=O)c2ccc(C(F)(F)F)cc12] |
| (I-24) | (I-26) |
| 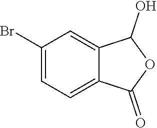 [SMILES: O=C1OC(O)c2cc(Br)ccc21] | 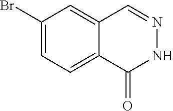 [SMILES: O=c1[nH]ncc2cc(Br)ccc12] |
| I-1026 | (I-1035) |
| 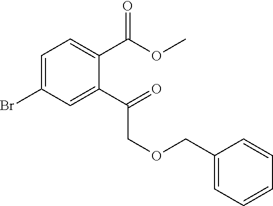 [SMILES: COC(=O)c1ccc(Br)cc1C(=O)COCc1ccccc1] | 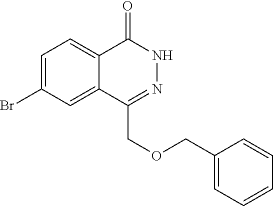 [SMILES: O=c1[nH]nc(COCc2ccccc2)c2cc(Br)ccc12] |
| I-1012 | (I-1036) |
| 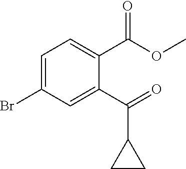 [SMILES: COC(=O)c1ccc(Br)cc1C(=O)C1CC1] | 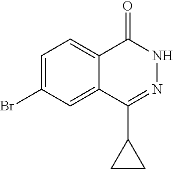 [SMILES: O=c1[nH]nc(C2CC2)c2cc(Br)ccc12] |
| I-1013 | (I-1037) |
| 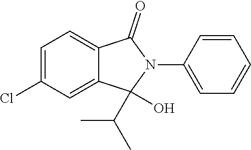 [SMILES: CC(C)C1(O)c2cc(Cl)ccc2C(=O)N1c1ccccc1] | 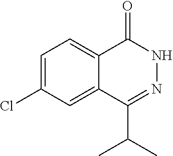 |
| I-1034 | (1038) |
| 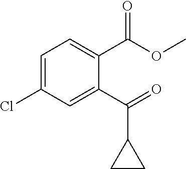 [SMILES: COC(=O)c1ccc(Cl)cc1C(=O)C1CC1] | 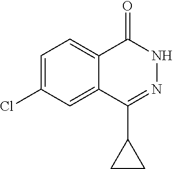 [SMILES: O=c1[nH]nc(C2CC2)c2cc(Cl)ccc12] |
| I-1015 | (I-1039) |
| 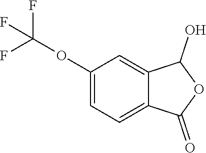 [SMILES: O=C1OC(O)c2cc(OC(F)(F)F)ccc21] | 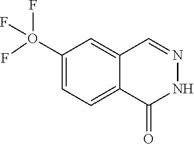 |
| I-1027 | (I-1040) |
| 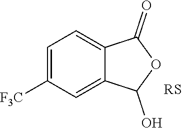 [SMILES: O=C1OC(O)c2cc(C(F)(F)F)ccc21] | 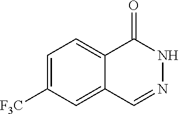 [SMILES: O=c1[nH]ncc2cc(C(F)(F)F)ccc12] |
| I-91 | I-83 |
| 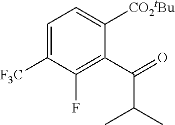 [SMILES: CC(C)C(=O)c1c(C(=O)OC(C)(C)C)ccc(C(F)(F)F)c1F] | 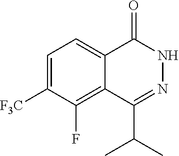 [SMILES: CC(C)c1n[nH]c(=O)c2ccc(C(F)(F)F)c(F)c12] |
| I-95 | I-86 |
| 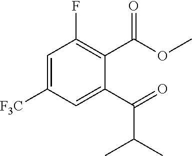 [SMILES: COC(=O)c1c(F)cc(C(F)(F)F)cc1C(=O)C(C)C] | 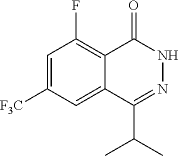 [SMILES: CC(C)c1n[nH]c(=O)c2c(F)cc(C(F)(F)F)cc12] |
| I-99 | I-100 |
| 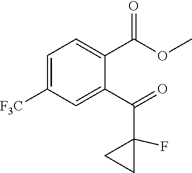 [SMILES: COC(=O)c1ccc(C(F)(F)F)cc1C(=O)C1(F)CC1] | 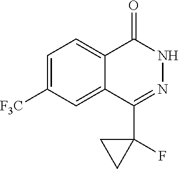 [SMILES: O=c1[nH]nc(C2(F)CC2)c2cc(C(F)(F)F)ccc12] |
| I-110 | I-109 |
| 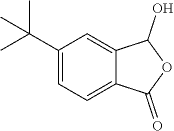 | 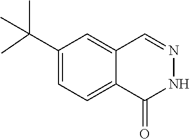 |
| I-1028 | I-1041 |
| 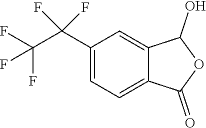 [SMILES: O=C1OC(O)c2cc(C(F)(F)C(F)(F)F)ccc21] | 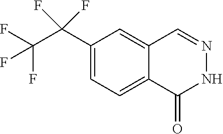 [SMILES: O=c1[nH]ncc2cc(C(F)(F)C(F)(F)F)ccc12] |
| I-1029 | I-1042 |
| 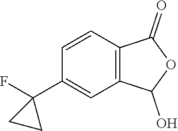 [SMILES: O=C1OC(O)c2cc(C3(F)CC3)ccc21] | 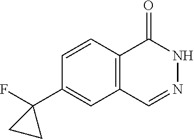 [SMILES: O=c1[nH]ncc2cc(C3(F)CC3)ccc12] |
| I-1030 | I-1043 |

### Synthesis of 4-isopropyl-1-oxo-6-(trifluoromethyl)-1,2-dihydrophthalazine-5-carbonitrile I-85

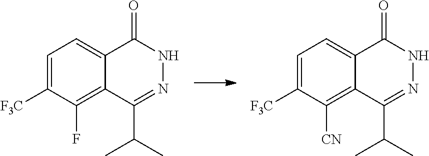 [SMILES: CC(C)c1n[nH]c(=O)c2ccc(C(F)(F)F)c(F)c12 ; CC(C)c1n[nH]c(=O)c2ccc(C(F)(F)F)c(C#N)c12]

KCN [151-50-8] (130.6 mg, 2.01 mmol) was added to a solution of 5-fluoro-4-isopropyl-6-(trifluoromethyl)phthalazin-1(2H)-one (1-86) (500 mg, 1.82 mmol) in DMSO (20 ml) at rt. The mixture was heated under MW irradiation at 150° C. for 40 min. The crude mixture was allowed to cool to rt and diluted with water and extracted with EtOAc (3×5 mL). The organic layer was evaporated in vacuo and purified by FCC (DCM/MeOH 0 to 5%) to afford (I-85) (420 mg, yield 82%) as a solid.

### Synthesis of 4,6-dibromophthalazin-1(2H)-one I-1044

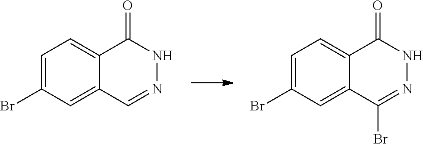 [SMILES: O=c1[nH]ncc2cc(Br)ccc12 ; O=c1[nH]nc(Br)c2cc(Br)ccc12]

K₂CO₃ [584-08-7] (3.07 g, 22.22 mmol) was added to a mixture of 6-bromophthalazin-1(2H)-one I-1035 (2.5 g, 11.11 mmol) in DMF (28 mL). The suspension was stirred at rt for 10 min. Then benzyltrimethylammonium tribromide [111865-47-5] (8.66 g, 22.22 mmol) was added. The reaction mixture was stirred at 40° C. for 5 h. Na2S2O3 sat. aq solution was added (until pH 7). The mixture was extracted with DCM. The organic layer was separated and dried over MgSO4 anh, filtered and solvent was concentrated in vacuo. The product was co-distillated with toluene (×5) to yield 4,6-dibromophthalazin-1(2H)-one I-1044 (685 mg, 19%) as a beige solid. The aq. layer was extracted with DCM:MeOH (9:1). The organic layer was separated and dried over MgSO4 anh, filtered and solvent was concentrated in vacuo. The product was co-distillated with toluene to yield 4,6-dibromophthalazin-1(2H)-one I-1044 (2.32 g, 67%) as a beige solid.

Structural analogs were synthesized using the same procedure.

| Intermediate | Product |
|---|---|
| 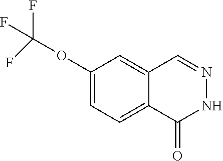 [SMILES: O=c1[nH]ncc2cc(OC(F)(F)F)ccc12] | 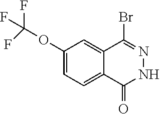 [SMILES: O=c1[nH]nc(Br)c2cc(OC(F)(F)F)ccc12] |
| (I-1040) | I-1045 |
| 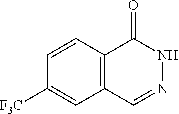 [SMILES: O=c1[nH]ncc2cc(C(F)(F)F)ccc12] (I-83) | 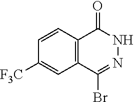 [SMILES: O=c1[nH]nc(Br)c2cc(C(F)(F)F)ccc12] |
| | (I-82) |
| 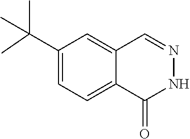 | 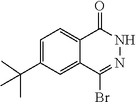 [SMILES: CC(C)(C)c1ccc2c(=O)[nH]nc(Br)c2c1] |
| I-1041 | I-1046 |
| 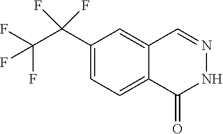 [SMILES: O=c1[nH]ncc2cc(C(F)(F)C(F)(F)F)ccc12] | 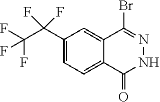 [SMILES: O=c1[nH]nc(Br)c2cc(C(F)(F)C(F)(F)F)ccc12] |
| I-1042 | I-1047 |
| 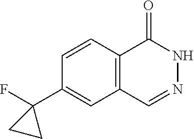 [SMILES: O=c1[nH]ncc2cc(C3(F)CC3)ccc12] | 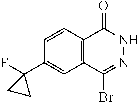 [SMILES: O=c1[nH]nc(Br)c2cc(C3(F)CC3)ccc12] |
| I-1043 | I-1048 |

### Synthesis of 4-isopropyl-1-oxo-6-(trifluoromethyl)-1,2-dihydrophthalazine-5-carbonitrile I-105

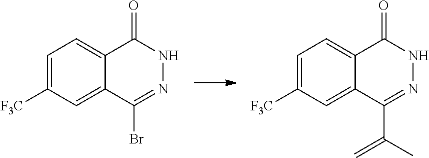 [SMILES: O=c1[nH]nc(Br)c2cc(C(F)(F)F)ccc12 ; C=C(C)c1n[nH]c(=O)c2ccc(C(F)(F)F)cc12]

A solution of 4-bromo-6-(trifluoromethyl)phthalazin-1(2H)-one I-82 (4 g, 13.65 mmol) in 1,4-dioxane (89.7 mL) and DI water (29.9 mL) was placed in an Easymax pressure tube and degassed with nitrogen during 15 min. Then, 4,4,5,5-tetramethyl-2-(prop-1-en-2-yl)-1,3,2-dioxoborolane [126726-62-3] (3.89 mL, 0.89 g/mL, 20.67 mmol)), K₃PO₄ [7778-53-2] (8.85 g, 41.69 mmol) and RuPhos Pd G3 [1445085-77-7] (0.6 g, 0.71 mmol) were added and the reaction mixture was stirred at 100° C. for 4 hours. The reaction was quenched with brine (˜400 mL) and the product was extracted with EtOAc (3×200 mL). The combined organic extracts were dried over MgSO₄, filtered and concentrated under reduced pressure at 40° C. The crude compound was purified by FCC (Het/EtOAc 0 to 30%) to afford I-105 (3 g, 86%) as a white solid.

Structural analogs were synthesized using the same procedure.

| Intermediate | Product |
|---|---|
| 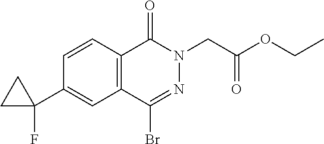 [SMILES: CCOC(=O)Cn1nc(Br)c2cc(C3(F)CC3)ccc2c1=O] | 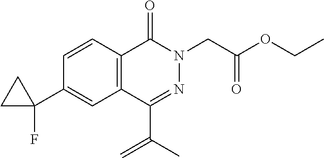 [SMILES: C=C(C)c1nn(CC(=O)OCC)c(=O)c2ccc(C3(F)CC3)cc12] |
| I-1049 | I-1050 |

### Synthesis of 4-isopropyl-1-oxo-6-(trifluoromethyl)-1,2-dihydrophthalazine-5-carbonitrile I-104

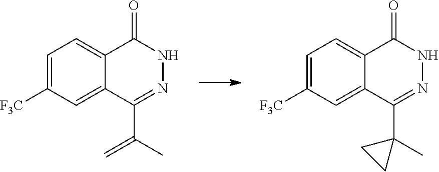 [SMILES: C=C(C)c1n[nH]c(=O)c2ccc(C(F)(F)F)cc12 ; CC1(c2n[nH]c(=O)c3ccc(C(F)(F)F)cc23)CC1]

A 1M solution of diethylzinc in hexanes [557-20-0] (22.6 mL, 22.6 mmol) was added in a flask containing dry DCM (20 mL) under nitrogen atmosphere and it was stirred at 0° C. TFA [76-05-1] (1.73 mL, 1.49 g/mL, 22.6 mmol) was added dropwise over 20 min via syringe pump. The reaction mixture was stirred at 0° C. for 20 min and diiodomethane [75-11-6] (1.82 mL, 3.33 g/mL, 22.6 mmol) was added over 20 min via syringe pump. The mixture was stirred at 0° C. for further 20 min and a solution of 4-(prop-1-en-2-yl)-6-(trifluoromethyl)phthalazin-1(2H)-one I-105 (500 mg, 1.97 mmol) in DCM (10 mL) was added over 20 min via syringe pump. After addition, the reaction mixture was allowed to warm to rt and stirred for additional 2 hours. The reaction was quenched by addition of a saturated aqueous solution of NH₄Cl and the solids were filtered. The organic layer was separated, dried on MgSO₄ and concentrated. A purification was performed via preparative HPLC (Stationary phase: RP XBridge Prep C18 OBD-10 μm, 50×250 mm, Mobile phase: 0.25% NH₄HCO₃ solution in water, CH₃CN) yielding I-104 (302 mg, 57%) as a white solid.

### Synthesis of 4-(1-ethoxyvinyl)-6-(trifluoromethyl)phthalazin-1(2H)-one I-116

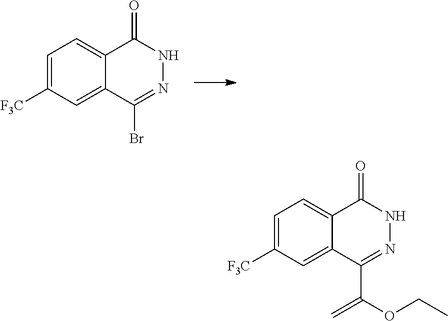 [SMILES: O=c1[nH]nc(Br)c2cc(C(F)(F)F)ccc12 ; C=C(OCC)c1n[nH]c(=O)c2ccc(C(F)(F)F)cc12]

Bis(triphenylphosphine)palladium(II) dichloride [13965-03-2] (244.4 mg, 0.34 mmol) and tributyl(1-ethoxyvinyl)tin [97674-02-7] (1.43 mL, 1.07 g/mL, 4.1 mmol) were added to a stirred solution of 4-bromo-6-(trifluoromethyl)phthalazin-1(2H)-one I-82 (1 g, 3.41 mmol) in dry 1,4-dioxane under nitrogen in a sealed tube. The mixture was stirred at 100° C. for 16 hours. The mixture was diluted with a saturated aqueous solution of NaHCO₃ and extracted with EtOAc. The organic layer was separated, dried (MgSO₄), filtered and the volatiles concentrated under vacuum. The crude product was purified by FCC (Hept/EtOAcO to 30%) to yield I-116 (948 mg, 95% purity, 93%) as a pale yellow solid.

### Synthesis of 4-acetyl-6-(trifluoromethyl)phthalazin-1(2H)-one I-115

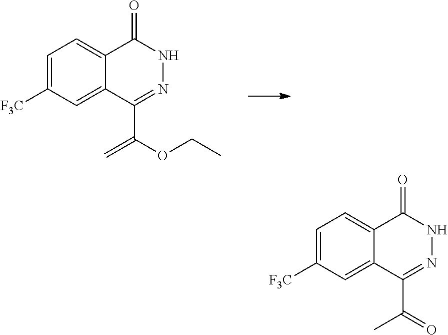 [SMILES: C=C(OCC)c1n[nH]c(=O)c2ccc(C(F)(F)F)cc12 ; CC(=O)c1n[nH]c(=O)c2ccc(C(F)(F)F)cc12]

6M aqueous HCl [7647-01-0] (2.77 mL, 16.6 mmol) was added dropwise to a stirred solution of 4-(1-ethoxyvinyl)-6-(trifluoromethyl)phthalazin-1(2H)-one I-116 (945 mg, 3.32 mmol) in 1,4-dioxane at 0° C. The mixture was stirred at rt for 1 hour. It was then diluted with a saturated aqueous solution of NaHCO₃ and extracted with EtOAc. The organic layer was separated, dried (MgSO₄), filtered and the volatiles evaporated in vacuo to yield I-115 (842 mg, 95% purity, 94%) as a white solid.

### Synthesis of 4-(2-hydroxypropan-2-yl)-6-(trifluoromethyl)phthalazin-1(2H)-one I-114

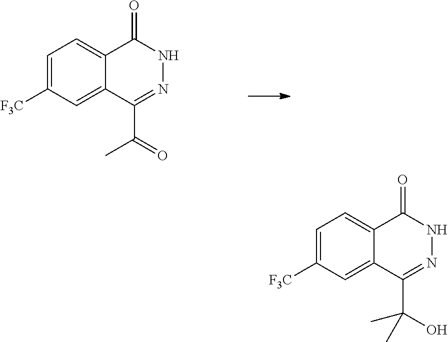 [SMILES: CC(=O)c1n[nH]c(=O)c2ccc(C(F)(F)F)cc12 ; CC(C)(O)c1n[nH]c(=O)c2ccc(C(F)(F)F)cc12]

A 1.4M solution of methylmagnesium bromide in THF [75-16-1] (7 mL, 9.83 mmol) was added dropwise to a stirred solution of 4-acetyl-6-(trifluoromethyl)phthalazin-1(2H)-one I-115 (839 mg, 3.28 mmol) in dry THF (21.3 mL) at 0° C. under nitrogen. The mixture was then stirred at 5° C. for 1 hour. It was diluted with a saturated aqueous solution of NaHCO₃ and extracted with EtOAc. The organic layer was separated, dried (MgSO₄), filtered and the volatiles evaporated in vacuo. The crude product was purified by FCC (Hept/EtOAc 0 to 30%) to yield I-114 (576 mg, 97-98% purity, 64%) as an off-white solid.

| Intermediate | Product |
|---|---|
| 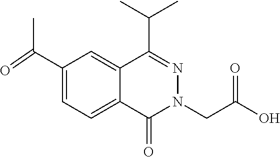 [SMILES: CC(=O)c1ccc2c(=O)n(CC(=O)O)nc(C(C)C)c2c1] | 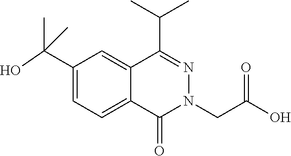 [SMILES: CC(C)c1nn(CC(=O)O)c(=O)c2ccc(C(C)(C)O)cc12] |
| I-1051 | I-1052 |
| 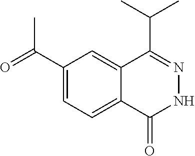 [SMILES: CC(=O)c1ccc2c(=O)[nH]nc(C(C)C)c2c1] | 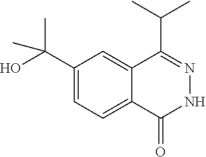 [SMILES: CC(C)c1n[nH]c(=O)c2ccc(C(C)(C)O)cc12] |
| I-1053 | I-1054 |

### Synthesis of 4-(1-methoxycyclopropyl)-6-(trifluoromethyl)phthalazin-1(2H)-one I-126

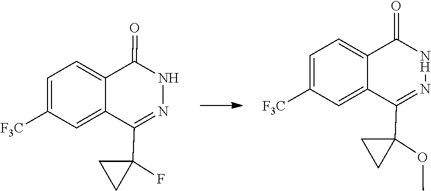 [SMILES: O=c1[nH]nc(C2(F)CC2)c2cc(C(F)(F)F)ccc12 ; COC1(c2n[nH]c(=O)c3ccc(C(F)(F)F)cc23)CC1]

A mixture of 4-(1-fluorocyclopropyl)-6-(trifluoromethyl)phthalazin-1(2H)-one I-109 (300 mg, 1.1 mmol) and 0.5M NaOMe in MeOH [124-41-4] (22 mL, 11 mmol) was stirred under a nitrogen atmosphere and heated in a pressure tube at 120° C. for 12 hours. The solvent was evaporated and the residue taken in DI water. NH₄Cl [12125-02-9] (1 g) was added and the mixture extracted with EtOAc. The combined organic extracts were washed with brine, dried on MgSO₄ and evaporated. The residue was purified by FCC (DCM/MeOH 0 to 5%) yielding I-126 (200 mg, 64%) as a white solid.

### Synthesis of ethyl 2-(4-isopropyl-6-methoxy-1-oxo-phthalazin-2-yl)acetate (1-21)

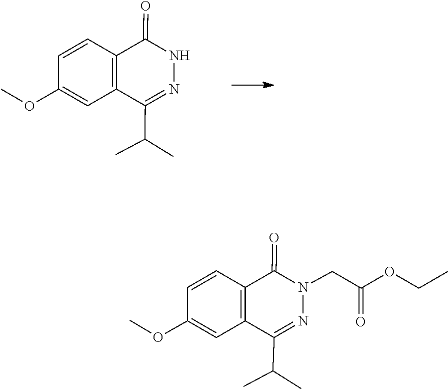 [SMILES: COc1ccc2c(=O)[nH]nc(C(C)C)c2c1 ; CCOC(=O)Cn1nc(C(C)C)c2cc(OC)ccc2c1=O]

Ethyl chloroacetate [105-39-5] (80 μL, 0.85 mmol) was added to a stirred mixture of 4-isopropyl-6-methoxy-2H-phthalazin-1-one (1-12) (148 mg, 0.68 mmol), 18-crown-6 [17455-13-9] (11 mg, 0.042 mmol), potassium iodide [7681-11-0] (17 mg, 0.1 mmol), K₂CO₃ [584-08-7] (118 mg, 0.85 mmol) in anhydrous ACN (6 mL) and DCM (2 mL) in a sealed tube. The mixture was stirred at 90° C. for 5 h. The mixture was treated with water and brine and then extracted with DCM. The organic layer was separated, dried (MgSO4), filtered and the solvents evaporated in vacuo. The crude product was purified by flash column chromatography (silica, EtOAc in heptane 0/100 to 30/70). The desired fractions were collected and concentrated in vacuo to yield ethyl 2-(4-isopropyl-6-methoxy-1-oxo-phthalazin-2-yl)acetate (1-21) (163 mg, 79%) as a white solid.

Structural analogs were synthesized using the same procedure.

| Reagent | Starting material | Intermediate |
|---|---|---|
| 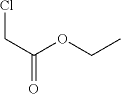 [105-39-5] | 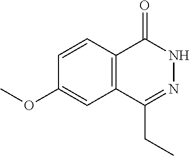 | 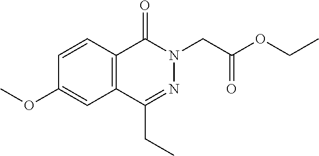 [SMILES: CCOC(=O)Cn1nc(CC)c2cc(OC)ccc2c1=O] |
| | (I-13) | (I-22) |
| 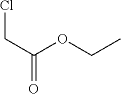 [105-39-5] | 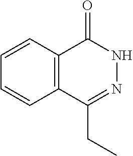 | 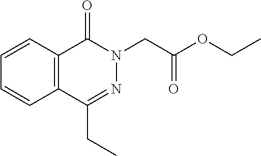 [SMILES: CCOC(=O)Cn1nc(CC)c2ccccc2c1=O] |
| | [54145-30-1] | (I-23) |
| 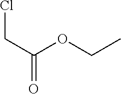 [105-39-5] | 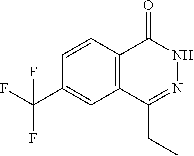 [SMILES: CCc1n[nH]c(=O)c2ccc(C(F)(F)F)cc12] | 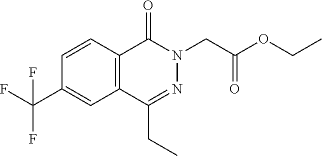 [SMILES: CCOC(=O)Cn1nc(CC)c2cc(C(F)(F)F)ccc2c1=O] |
| | (I-18) | (I-25) |
| 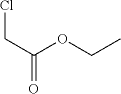 [105-39-5] | 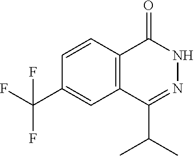 [SMILES: CC(C)c1n[nH]c(=O)c2ccc(C(F)(F)F)cc12] | 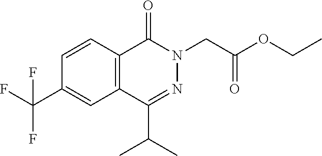 [SMILES: CCOC(=O)Cn1nc(C(C)C)c2cc(C(F)(F)F)ccc2c1=O] |
| | (I-26) | (I-29) |
| 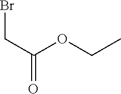 [105-36-2] Cs2CO3 [534-17-8] | 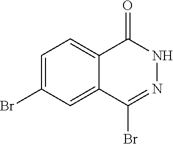 [SMILES: O=c1[nH]nc(Br)c2cc(Br)ccc12] I-1044 | 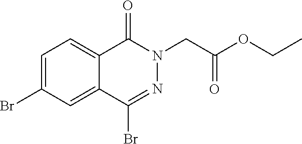 [SMILES: CCOC(=O)Cn1nc(Br)c2cc(Br)ccc2c1=O] I-1055 |
| 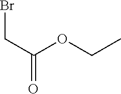 [105-36-2] | 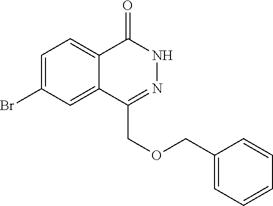 [SMILES: O=c1[nH]nc(COCc2ccccc2)c2cc(Br)ccc12] | 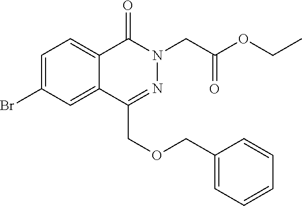 [SMILES: CCOC(=O)Cn1nc(COCc2ccccc2)c2cc(Br)ccc2c1=O] |
| | (I-1036) | I-1056 |
| 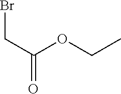 [105-36-2] Cs2CO3 [534-17-8] | 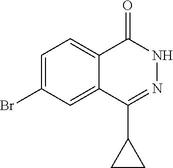 [SMILES: O=c1[nH]nc(C2CC2)c2cc(Br)ccc12] | 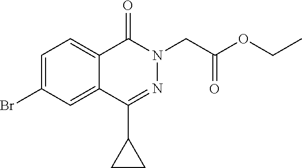 [SMILES: CCOC(=O)Cn1nc(C2CC2)c2cc(Br)ccc2c1=O] |
| | (I-1037) | I-1057 |
| 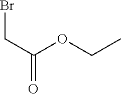 [105-36-2] | 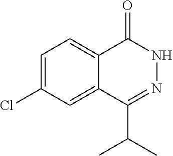 | 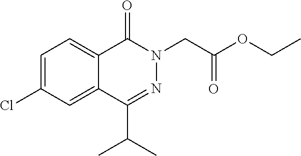 [SMILES: CCOC(=O)Cn1nc(C(C)C)c2cc(Cl)ccc2c1=O] |
| | (I-1038) | I-1058 |
| 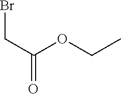 [105-36-2] | 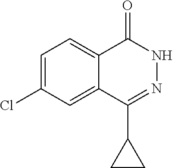 [SMILES: O=c1[nH]nc(C2CC2)c2cc(Cl)ccc12] | 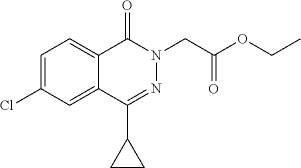 [SMILES: CCOC(=O)Cn1nc(C2CC2)c2cc(Cl)ccc2c1=O] |
| | (I-1039) | I-1059 |
| 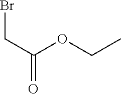 [105-36-2] Cs2CO3 [534-17-8] | 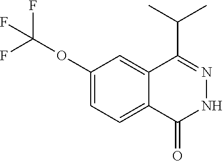 [SMILES: CC(C)c1n[nH]c(=O)c2ccc(OC(F)(F)F)cc12] | 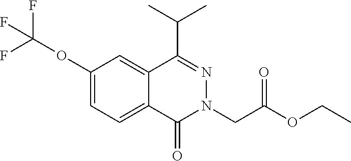 [SMILES: CCOC(=O)Cn1nc(C(C)C)c2cc(OC(F)(F)F)ccc2c1=O] |
| | I-1060 | I-1061 |
| 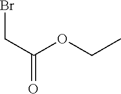 [105-36-2] | 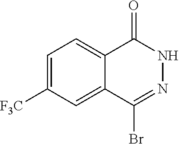 [SMILES: O=c1[nH]nc(Br)c2cc(C(F)(F)F)ccc12] | 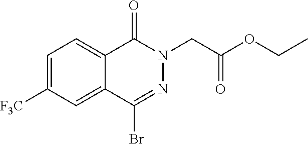 [SMILES: CCOC(=O)Cn1nc(Br)c2cc(C(F)(F)F)ccc2c1=O] |
| | I-82 | I-81 |
| 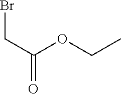 [105-36-2] | 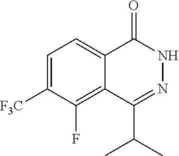 [SMILES: CC(C)c1n[nH]c(=O)c2ccc(C(F)(F)F)c(F)c12] | 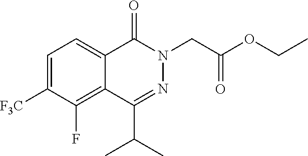 [SMILES: CCOC(=O)Cn1nc(C(C)C)c2c(F)c(C(F)(F)F)ccc2c1=O] |
| | I-86 | I-88 |
| 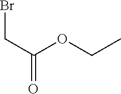 [105-36-2] | 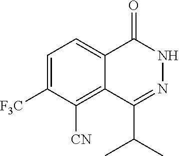 [SMILES: CC(C)c1n[nH]c(=O)c2ccc(C(F)(F)F)c(C#N)c12] | 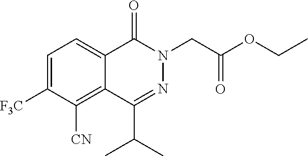 [SMILES: CCOC(=O)Cn1nc(C(C)C)c2c(C#N)c(C(F)(F)F)ccc2c1=O] |
| | I-85 | I-87 |
| 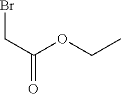 [105-36-2] | 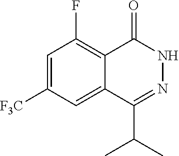 [SMILES: CC(C)c1n[nH]c(=O)c2c(F)cc(C(F)(F)F)cc12] | 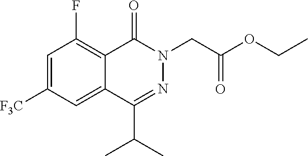 [SMILES: CCOC(=O)Cn1nc(C(C)C)c2cc(C(F)(F)F)cc(F)c2c1=O] |
| | I-100 | I-101 |
| 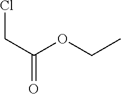 [105-39-5] | 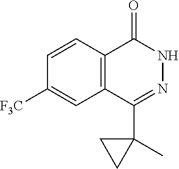 [SMILES: CC1(c2n[nH]c(=O)c3ccc(C(F)(F)F)cc23)CC1] | 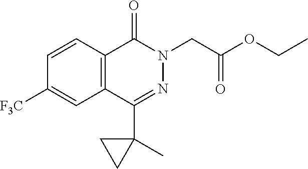 [SMILES: CCOC(=O)Cn1nc(C2(C)CC2)c2cc(C(F)(F)F)ccc2c1=O] |
| | (I-103) | (I-104) |
| 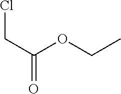 [105-39-5] | 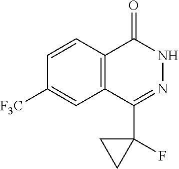 [SMILES: O=c1[nH]nc(C2(F)CC2)c2cc(C(F)(F)F)ccc12] | 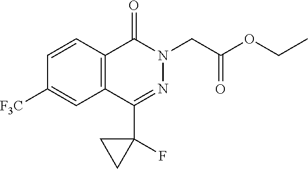 [SMILES: CCOC(=O)Cn1nc(C2(F)CC2)c2cc(C(F)(F)F)ccc2c1=O] |
| | (I-109) | (I-108) |
| 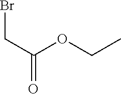 [105-36-2] | 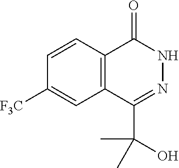 [SMILES: CC(C)(O)c1n[nH]c(=O)c2ccc(C(F)(F)F)cc12] | 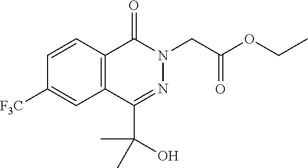 [SMILES: CCOC(=O)Cn1nc(C(C)(C)O)c2cc(C(F)(F)F)ccc2c1=O] |
| | (I-114) | (I-113) |
| 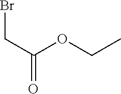 [105-36-2] | 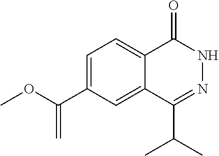 [SMILES: C=C(OC)c1ccc2c(=O)[nH]nc(C(C)C)c2c1] | 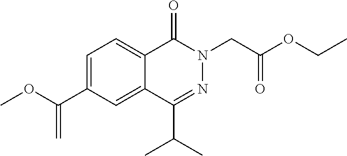 [SMILES: C=C(OC)c1ccc2c(=O)n(CC(=O)OCC)nc(C(C)C)c2c1] |
| | I-1062 | I-1063 |
| 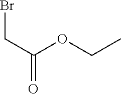 [105-36-2] | 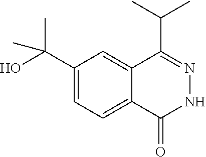 [SMILES: CC(C)c1n[nH]c(=O)c2ccc(C(C)(C)O)cc12] | 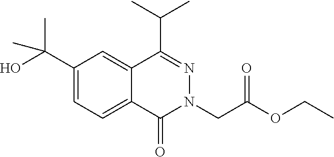 [SMILES: CCOC(=O)Cn1nc(C(C)C)c2cc(C(C)(C)O)ccc2c1=O] |
| | I-1054 | I-1064 |
| 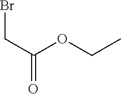 [105-36-2] Cs2CO3 [534-17-8] | 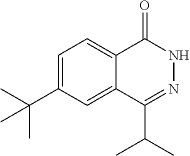 [SMILES: CC(C)c1n[nH]c(=O)c2ccc(C(C)(C)C)cc12] | 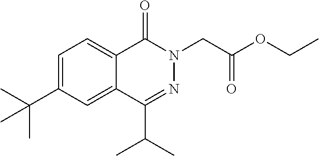 [SMILES: CCOC(=O)Cn1nc(C(C)C)c2cc(C(C)(C)C)ccc2c1=O] |
| | I-1065 | I-1066 |
| 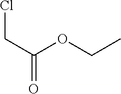 [105-39-5] | 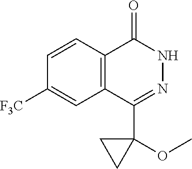 [SMILES: COC1(c2n[nH]c(=O)c3ccc(C(F)(F)F)cc23)CC1] | 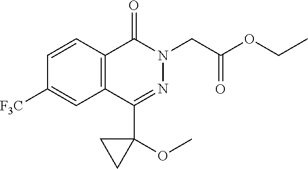 [SMILES: CCOC(=O)Cn1nc(C2(OC)CC2)c2cc(C(F)(F)F)ccc2c1=O] |
| | (I-126) | (I-127) |
| 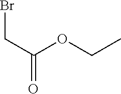 [105-36-2] Cs2CO3 [534-17-8] | 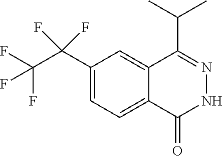 [SMILES: CC(C)c1n[nH]c(=O)c2ccc(C(F)(F)C(F)(F)F)cc12] | 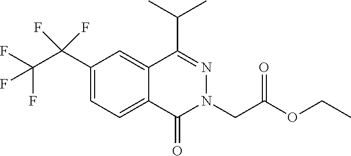 [SMILES: CCOC(=O)Cn1nc(C(C)C)c2cc(C(F)(F)C(F)(F)F)ccc2c1=O] |
| | I-1067 | I-1068 |
| 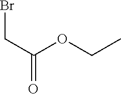 [105-36-2] Cs2CO3 [534-17-8] | 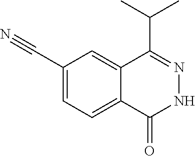 [SMILES: CC(C)c1n[nH]c(=O)c2ccc(C#N)cc12] | 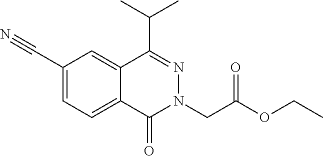 [SMILES: CCOC(=O)Cn1nc(C(C)C)c2cc(C#N)ccc2c1=O] |
| | I-1069 | I-1070 |
| 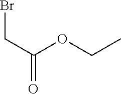 [105-36-2] | 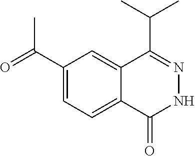 [SMILES: CC(=O)c1ccc2c(=O)[nH]nc(C(C)C)c2c1] | 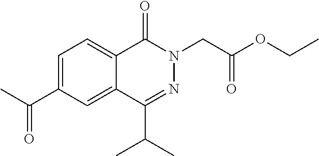 [SMILES: CCOC(=O)Cn1nc(C(C)C)c2cc(C(C)=O)ccc2c1=O] |
| | I-1053 | I-1071 |
| 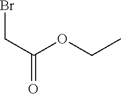 [105-36-2] Cs2CO3 [534-17-8] | 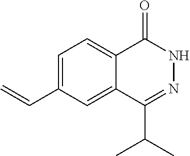 [SMILES: C=Cc1ccc2c(=O)[nH]nc(C(C)C)c2c1] | 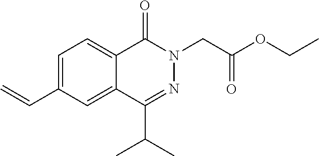 [SMILES: C=Cc1ccc2c(=O)n(CC(=O)OCC)nc(C(C)C)c2c1] |
| | I-1072 | I-1073 |
| 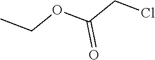 [105-39-5] | 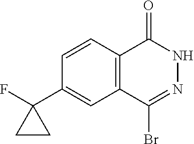 [SMILES: O=c1[nH]nc(Br)c2cc(C3(F)CC3)ccc12] | 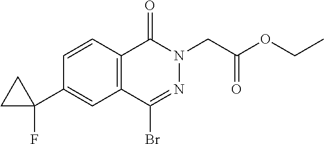 [SMILES: CCOC(=O)Cn1nc(Br)c2cc(C3(F)CC3)ccc2c1=O] |
| | I-1048 | I-1049 |

### Synthesis of ethyl 2-(7-bromo-4-ethyl-1-oxo-phthalazin-2-yl)acetate (I-28)

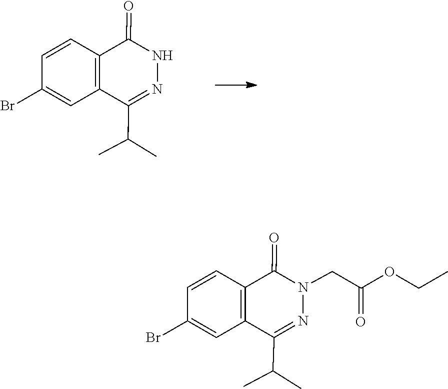 [SMILES: CC(C)c1n[nH]c(=O)c2ccc(Br)cc12 ; CCOC(=O)Cn1nc(C(C)C)c2cc(Br)ccc2c1=O]

Ethyl bromoacetate [105-36-2] (0.82 mL, 7.4 mmol) was added to a stirred suspension of 7-bromo-4-ethyl-2H-phthalazin-1-one (1-14) (2.6 g, 6.16 mmol, 60% purity) and NaH [7646-69-7] (60% dispersion in mineral oil, 0.27 g, 6.78 mmol) in anhydrous DMF (24.6 mL) at 0° C. The mixture was stirred at room temperature for 1 h. The mixture was diluted with water and extracted with EtOAc. The organic layer was separated, dried (MgSO4), filtered and the solvents evaporated in vacuo. The crude product was purified by flash column chromatography (silica, EtOAc in heptane 0/100 to 20/80). The desired fractions were collected and concentrated in vacuo to yield ethyl 2-(7-bromo-4-ethyl-1-oxo-phthalazin-2-yl)acetate (1-28) (1.15 g, 55%) as pale yellow oil.

Structural analogs were synthesized using the same procedure.

| Starting material | Intermediate |
|---|---|
| 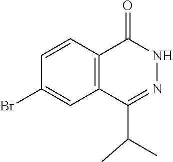 (I-15) | 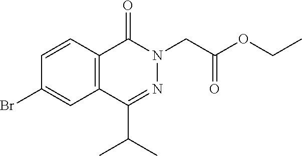 [SMILES: CCOC(=O)Cn1nc(C(C)C)c2cc(Br)ccc2c1=O] (I-31) |
| 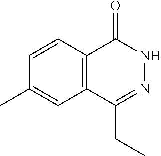 | 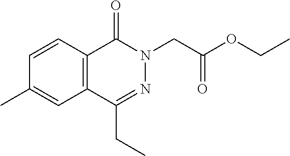 [SMILES: CCOC(=O)Cn1nc(CC)c2cc(C)ccc2c1=O] |
| (I-16) | (I-30) |

### Synthesis of ethyl 2-(6-bromo-4-(hydroxymethyl)-1-oxophthalazin-2(1H)-yl)acetate I-1031

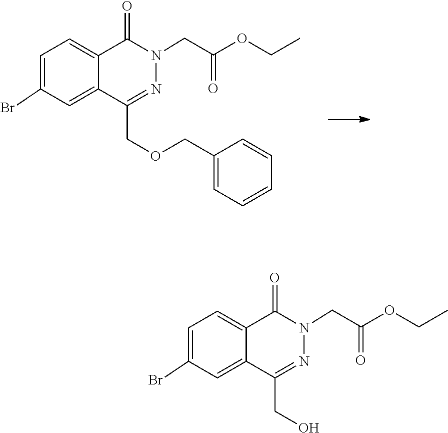 [SMILES: CCOC(=O)Cn1nc(COCc2ccccc2)c2cc(Br)ccc2c1=O ; CCOC(=O)Cn1nc(CO)c2cc(Br)ccc2c1=O]

Chlorotrimethylsilane [75-77-4] (1.37 mL, 0.86 g/mL, 10.7 mmol) and sodium iodide [7681-82-5] (1.62 g, 10.7 mmol) were added to a stirred solution of ethyl 2-(4-((benzyloxy)methyl)-6-bromo-1-oxophthalazin-2(1H)-yl)acetate I-1056 (2.23 g, 4.65 mmol) in acetonitrile anhydrous (24 mL) at rt under nitrogen atmosphere. The mixture was stirred at 80° C. for 7 h. The mixture was diluted with sat. aqueous NaHCO₃ (32 mL) and 10% aqueous Na2S203 (32 mL) and extracted with AcOEt. The organic layer was separated, dried (MgSO4), filtered and the solvents evaporated in vacuo. The crude product was purified by flash column chromatography (silica; AcOEt in heptane 0/100 to 35/65). The desired fractions were collected and concentrated in vacuo to yield ethyl 2-(6-bromo-4-(hydroxymethyl)-1-oxophthalazin-2(1H)-yl)acetate I-1031 (523 mg, 33%) as a white solid.

### Synthesis of ethyl 2-(6-bromo-4-(1-ethoxyvinyl)-1-oxophthalazin-2(1H)-yl)acetate I-1074

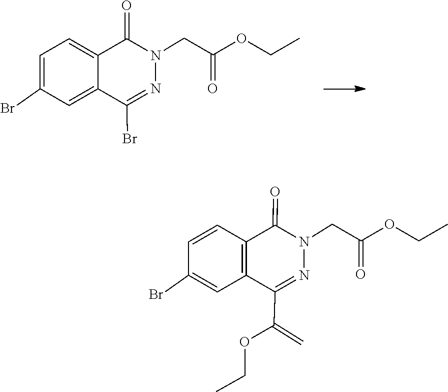 [SMILES: CCOC(=O)Cn1nc(Br)c2cc(Br)ccc2c1=O ; C=C(OCC)c1nn(CC(=O)OCC)c(=O)c2ccc(Br)cc12]

Bis(triphenylphosphine)palladium(II) dichloride [13965-03-2] (92 mg, 0.13 mmol) and tributyl (1-ethoxyvinyl)tin [97674-02-7] (402 μL, 1.07 g/mL, 1.15 mmol) were added to a stirred solution of ethyl 2-(4,6-dibromo-1-oxophthalazin-2(1H)-yl)acetate I-1055 (500 mg, 1.28 mmol) in toluene (10 mL) under nitrogen atmosphere in a sealed tube. The mixture was stirred at 80° C. for 4 h. The mixture was diluted with sat. aqueous NaHCO3 and extracted with AcOEt. The organic layer was separated, dried (MgSO4), filtered and the solvents evaporated in vacuo. The crude product was purified by flash column chromatography (silica, AcOEt in heptane 0/100 to 20/80). The desired fractions were collected and concentrated in vacuo to yield ethyl 2-(6-bromo-4-(1-ethoxyvinyl)-1-oxophthalazin-2(1H)-yl)acetate I-1074 (252 mg, 410%) as a yellow solid.

Structural analogues were synthesized using the same procedure.

| Reagent | Starting material | Intermediate |
|---|---|---|
| 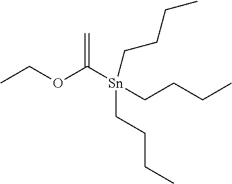 [SMILES: C=[C](OCC)[Sn]([CH2]CCC)([CH2]CCC)[CH2]CCC] [97674-02-7] | 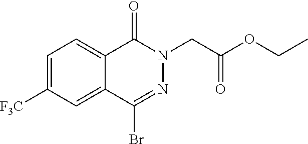 [SMILES: CCOC(=O)Cn1nc(Br)c2cc(C(F)(F)F)ccc2c1=O] I-81 | 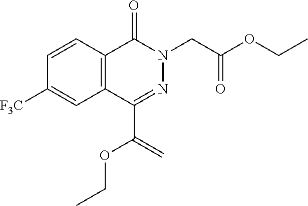 [SMILES: C=C(OCC)c1nn(CC(=O)OCC)c(=O)c2ccc(C(F)(F)F)cc12] I-80 |
| 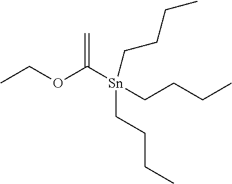 [SMILES: C=[C](OCC)[Sn]([CH2]CCC)([CH2]CCC)[CH2]CCC] | 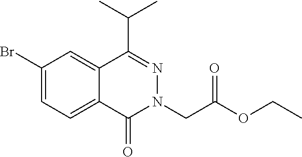 [SMILES: CCOC(=O)Cn1nc(C(C)C)c2cc(Br)ccc2c1=O] (I-31) | 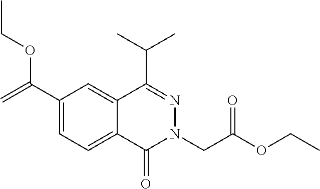 [SMILES: C=C(OCC)c1ccc2c(=O)n(CC(=O)OCC)nc(C(C)C)c2c1] |
| [97674-02-7] | | I-1075 |
| 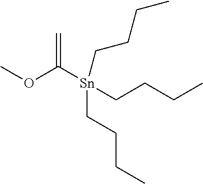 [SMILES: C=[C](OC)[Sn]([CH2]CCC)([CH2]CCC)[CH2]CCC] [81177-90-4] | 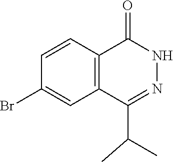 (I-15) | 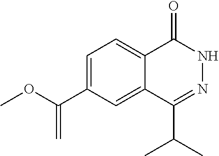 [SMILES: C=C(OC)c1ccc2c(=O)[nH]nc(C(C)C)c2c1] I-1062 |
| 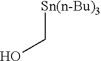 I-117 | 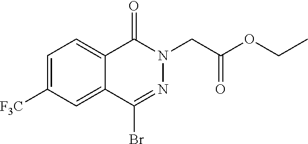 [SMILES: CCOC(=O)Cn1nc(Br)c2cc(C(F)(F)F)ccc2c1=O] I-81 | 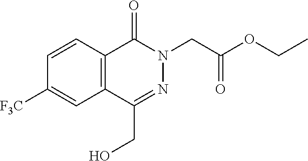 [SMILES: CCOC(=O)Cn1nc(CO)c2cc(C(F)(F)F)ccc2c1=O] |
| | | I-118 |
| 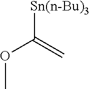 [81177-90-4] | 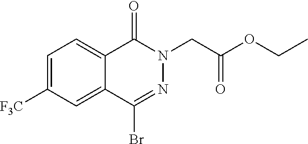 [SMILES: CCOC(=O)Cn1nc(Br)c2cc(C(F)(F)F)ccc2c1=O] I-81 | 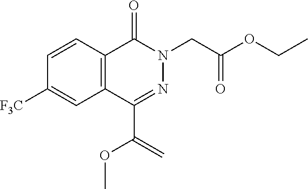 [SMILES: C=C(OC)c1nn(CC(=O)OCC)c(=O)c2ccc(C(F)(F)F)cc12] |
| | | I-121 |
| 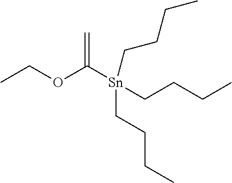 [SMILES: C=[C](OCC)[Sn]([CH2]CCC)([CH2]CCC)[CH2]CCC] [97674-02-7] | 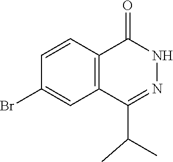 (I-15) | 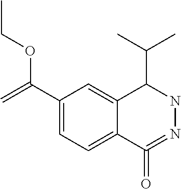 I-1076 |

### Synthesis of ethyl 2-(6-(1-ethoxyethyl)-4-isopropyl-1-oxophthalazin-2(1H)-yl)acetate I-1077

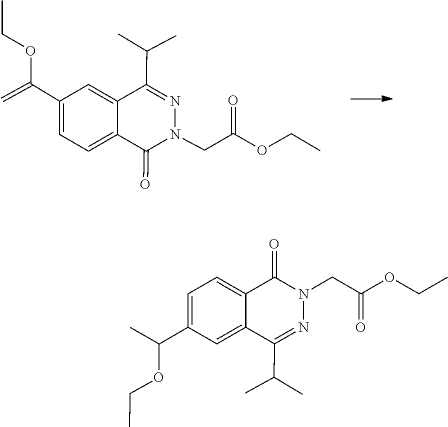

Tris(triphenylphosphine)rhodium(I) chloride [14694-95-2] (31 mg, 0.034 mmol) was added to a stirred solution of ethyl 2-(6-(1-ethoxyvinyl)-4-isopropyl-1-oxophthalazin-2(1H)-yl)acetate I-1075 (116 mg, 0.34 mmol) in ethanol (5 mL) at rt under nitrogen atmosphere. Then, nitrogen atmosphere was replaced by H2 (balloon) and the reaction mixture was stirred at rt for 16 h. Solvent was evaporated in vacuo. The crude product was purified by flash column chromatography (silica 12 g; AcOEt in heptane 0/100 to 20/80). The desired fractions were collected and concentrated in vacuo to yield ethyl 2-(6-(1-ethoxyethyl)-4-isopropyl-1-oxophthalazin-2(1H)-yl)acetate I-1077 (107 mg, 90%) as a colorless sticky solid.

| Intermediate | Product |
|---|---|
| 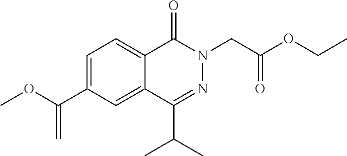 [SMILES: C=C(OC)c1ccc2c(=O)n(CC(=O)OCC)nc(C(C)C)c2c1] | 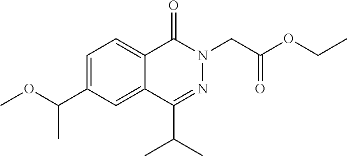 [SMILES: CCOC(=O)Cn1nc(C(C)C)c2cc(C(C)OC)ccc2c1=O] |
| I-1063 | I-1078 |
| 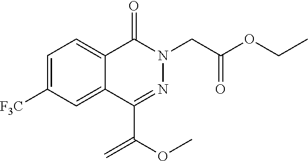 [SMILES: C=C(OC)c1nn(CC(=O)OCC)c(=O)c2ccc(C(F)(F)F)cc12] I-121 | 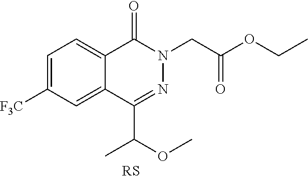 [SMILES: CCOC(=O)Cn1nc(C(C)OC)c2cc(C(F)(F)F)ccc2c1=O] |
| | I-122 |
| 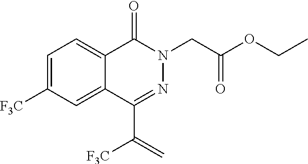 [SMILES: C=C(c1nn(CC(=O)OCC)c(=O)c2ccc(C(F)(F)F)cc12)C(F)(F)F] | 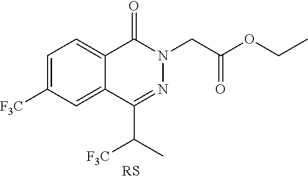 [SMILES: CCOC(=O)Cn1nc(C(C)C(F)(F)F)c2cc(C(F)(F)F)ccc2c1=O] |
| I-129 | I-128 |

### Synthesis of ethyl 2-(6-hydroxy-4-isopropyl-1-oxophthalazin-2(1H)-yl)acetate I-1079

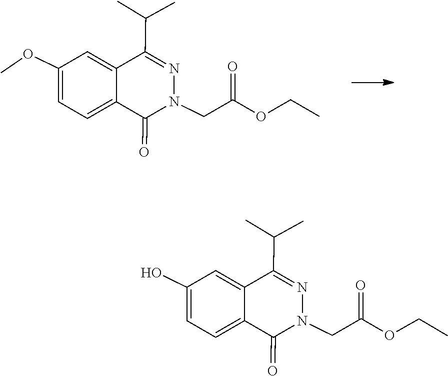 [SMILES: CCOC(=O)Cn1nc(C(C)C)c2cc(OC)ccc2c1=O ; CCOC(=O)Cn1nc(C(C)C)c2cc(O)ccc2c1=O]

Chlorotrimethylsilane [75-77-4] (250 μL, 1.95 mmol) and sodium iodide [7681-82-5](2.96 mg, 1.95 mmol) were added to a stirred solution of ethyl 2-(4-isopropyl-6-methoxy-1-oxophthalazin-2(1H)-yl)acetate (1-21) (300 mg, in acetonitrile anhydrous at rt under nitrogen atmosphere. The mixture was stirred at 80° C. for 5 h. Chlorotrimethylsilane [75-77-4] (250 μL, 1.95 mmol) and sodium iodide [7681-82-5](2.96 mg, 1.95 mmol) were added and the mixture was stirred at 80° C. for 16 h. The mixture was diluted with sat. aqueous NaHCO₃ (18 mL) and 10% aqueous Na2S203 (18 mL) and extracted with AcOEt. The organic layer was separated, dried (MgSO4), filtered and the solvents evaporated in vacuo. The crude product was purified by flash column chromatography (silica 25 g; AcOEt in heptane 0/100 to 100/0). The desired fractions were collected and concentrated in vacuo to yield ethyl 2-(6-hydroxy-4-isopropyl-1-oxophthalazin-2(1H)-yl)acetate I-1079 (55 mg, 21%) as a beige solid.

### Synthesis of ethyl 2-(4-isopropyl-1-oxo-6-(2,2,2-trifluoroethoxy)phthalazin-2(1H)-yl)acetate I-1080

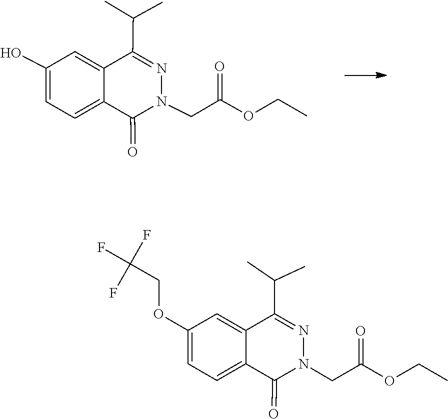 [SMILES: CCOC(=O)Cn1nc(C(C)C)c2cc(O)ccc2c1=O ; CCOC(=O)Cn1nc(C(C)C)c2cc(OCC(F)(F)F)ccc2c1=O]

2,2,2-Trifluoroethyl perfluorobutylsulfonate [79963-95-4] (47 μL, 1.68 g/mL, 0.21 mmol) was added to a stirred solution of ethyl 2-(6-hydroxy-4-isopropyl-1-oxophthalazin-2(1H)-yl)acetate I-1079 (55 mg, 0.19 mmol) and cesium carbonate [534-178] (93 mg, 0.28 mmol) in DMF (2 mL). The mixture was stirred at RT for 4 h. The mixture was diluted with sat. aqueous NaHCO₃ and extracted with AcOEt. The organic layer was separated, dried (MgSO4), filtered and the solvents evaporated in vacuo. The crude product was purified by flash column chromatography (silica 25 g; AcOEt in heptane 0/100 to 15/85). The desired fractions were collected and concentrated in vacuo to yield ethyl 2-(4-isopropyl-1-oxo-6-(2,2,2-trifluoroethoxy)phthalazin-2(1H)-yl)acetate I-1080 (36 mg, 51%) as a yellow solid

### Synthesis of ethyl 2-(4-bromo-6-(dimethylamino)-1-oxophthalazin-2(1H)-yl)acetate I-1081

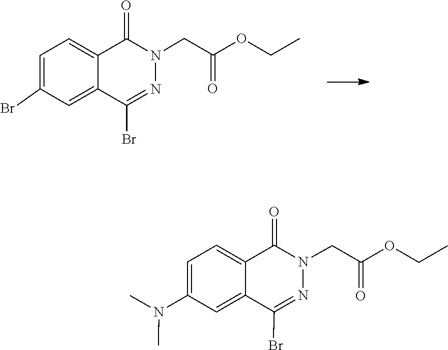

Dimethylamine solution 2M in THF [124-40-3] (1.92 mL, 3.85 mmol) was added to a stirred solution of ethyl 2-(4,6-dibromo-1-oxophthalazin-2(1H)-yl)acetate I-1055 (500 mg, 1.28 mmol) and N,N-diisopropylethylamine [7087-68-5] (1.35 mL, 0.74 g/mL, 7.69 mmol) in DMSO in a sealed tube. The mixture was stirred at 125° C. for 16 h. The mixture was diluted with water and extracted with AcOEt. The organic layer was washed with water (×3), separated, dried (MgSO4), filtered and the solvents evaporated in vacuo. The crude product was purified by flash column chromatography (silica; AcOEt in heptane 0/100 to 50/50). The desired fractions were collected and concentrated in vacuo to ethyl 2-(4-bromo-6-(dimethylamino)-1-oxophthalazin-2(1H)-yl)acetate I-1081 (142 mg, 31%) as a white solid.

Structural analogs were synthesized using the same procedure.

| Starting material | Intermediate |
|---|---|
| 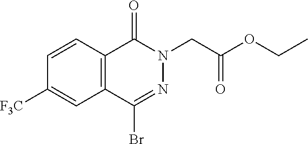 [SMILES: CCOC(=O)Cn1nc(Br)c2cc(C(F)(F)F)ccc2c1=O] (I-81) | 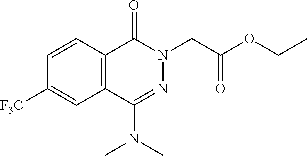 [SMILES: CCOC(=O)Cn1nc(N(C)C)c2cc(C(F)(F)F)ccc2c1=O] (I-155) |

### Synthesis of ethyl 2-(6-cyclopropyl-4-ethyl-1-oxo-phthalazin-2-yl)acetate (1-32)

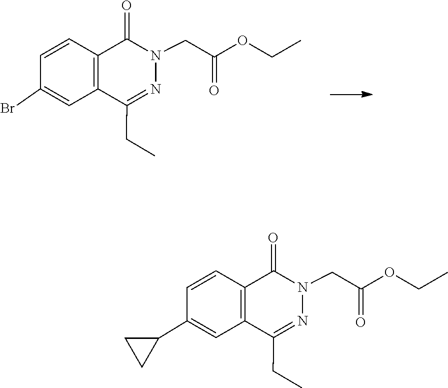 [SMILES: CCOC(=O)Cn1nc(CC)c2cc(Br)ccc2c1=O ; CCOC(=O)Cn1nc(CC)c2cc(C3CC3)ccc2c1=O]

[1,1′-Bis(diphenylphosphino)ferrocene]dichloropalladium(II), complex with dichloromethane [95464-05-4] (109 mg, 0.13 mmol) was added to a stirred suspension of ethyl 2-(6-bromo-4-ethyl-1-oxo-phthalazin-2-yl)acetate (1-28) (300 mg, 0.88 mmol), cyclopropyl boronic acid [411235-57-9] (190 mg, 2.21 mmol) and cesium carbonate [534-17-8] (0.63 g, 1.95 mmol) in a mixture of 1,4-dioxane (4 mL) and water (1 mL) under nitrogen. The mixture was stirred at 90° C. for 16 h. The cooled mixture was diluted with water and extracted with EtOAc. The organic layer was separated, dried (MgSO₄), filtered and the solvents evaporated in vacuo. The crude product was purified by flash column chromatography (silica; EtOAc in heptane 0/100 to 50/50). The desired fractions were collected and the solvents evaporated in vacuo to yield ethyl 2-(6-cyclopropyl-4-ethyl-1-oxo-phthalazin-2-yl)acetate (I-32) (129 mg, 48%) as yellow oil.

### Synthesis of ethyl 2-(4-ethyl-6-isopropenyl-1-oxo-phthalazin-2-yl)acetate (I-33)

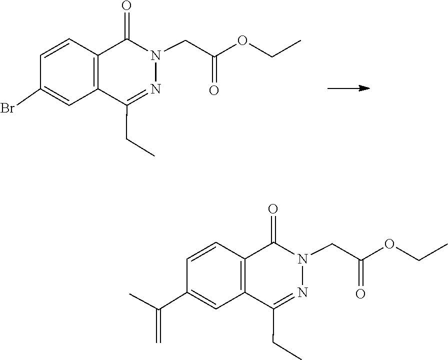

[1,1′-Bis(diphenylphosphino)ferrocene]dichloropalladium(II), complex with dichloromethane [95464-05-4] (0.15 g, 0.18 mmol) was added to a stirred suspension of ethyl 2-(6-bromo-4-ethyl-1-oxo-phthalazin-2-yl)acetate (I-28) (0.4 g, 1.18 mmol), potassium isopropenyltrifluoroborate [395083-14-4] (0.44 g, 2.95 mmol) and cesium carbonate [534-17-8] (0.84 g, 2.59 mmol) in a mixture of 1,4-dioxane (8 mL) and water (2 mL) under nitrogen. The mixture was stirred at 105° C. for 5 h. The cooled mixture was diluted with water and extracted with EtOAc. The organic layer was separated, washed with brine, dried (MgSO₄), filtered and the solvents evaporated in vacuo. The crude product was purified by flash column chromatography (silica; EtOAc in heptane 0/100 to 50/50). The desired fractions were collected and the solvents evaporated in vacuo to yield ethyl 2-(4-ethyl-6-isopropenyl-1-oxo-phthalazin-2-yl)acetate (I-33) (148 mg, 87%) as yellow oil.

Structural analogs were synthesized using the same procedure.

| Reagent | Starting material | Intermediate |
|---|---|---|
| 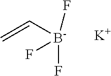 [13682-77-4] | 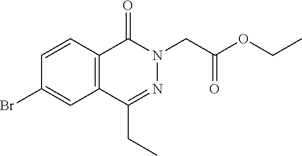 [SMILES: CCOC(=O)Cn1nc(CC)c2cc(Br)ccc2c1=O] | 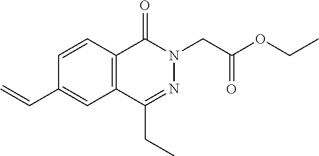 [SMILES: C=Cc1ccc2c(=O)n(CC(=O)OCC)nc(CC)c2c1] |
| | (I-28) | (I-34) |
| 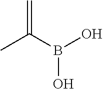 [126726-62-3] | 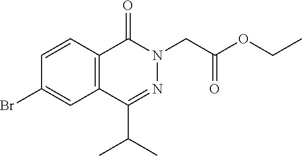 [SMILES: CCOC(=O)Cn1nc(C(C)C)c2cc(Br)ccc2c1=O] | 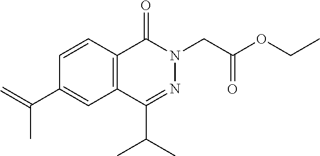 [SMILES: C=C(C)c1ccc2c(=O)n(CC(=O)OCC)nc(C(C)C)c2c1] |
| | (I-31) | (I-43) |
| 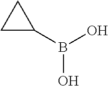 [411235-57-9] | 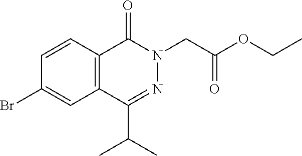 [SMILES: CCOC(=O)Cn1nc(C(C)C)c2cc(Br)ccc2c1=O] | 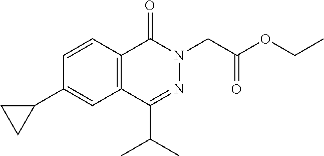 [SMILES: CCOC(=O)Cn1nc(C(C)C)c2cc(C3CC3)ccc2c1=O] |
| | (I-31) | (I-56) |
| 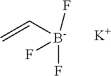 | 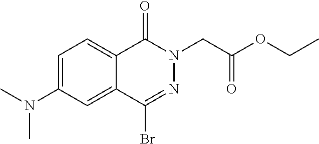 [SMILES: CCOC(=O)Cn1nc(Br)c2cc(N(C)C)ccc2c1=O] | 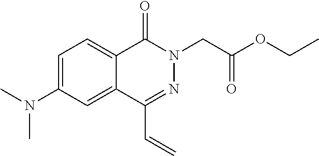 [SMILES: C=Cc1nn(CC(=O)OCC)c(=O)c2ccc(N(C)C)cc12] |
| [13682-77-4] | I-1081 | I-1082 |
| 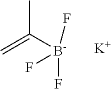 [395083-14-4] | 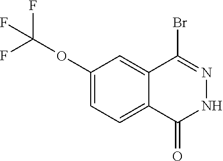 [SMILES: O=c1[nH]nc(Br)c2cc(OC(F)(F)F)ccc12] | 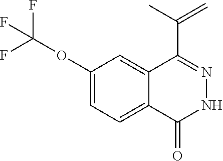 [SMILES: C=C(C)c1n[nH]c(=O)c2ccc(OC(F)(F)F)cc12] |
| | I-1045 | I-1083 |
| 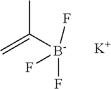 [395083-14-4] | 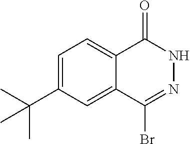 [SMILES: CC(C)(C)c1ccc2c(=O)[nH]nc(Br)c2c1] I-1046 | 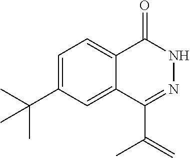 [SMILES: C=C(C)c1n[nH]c(=O)c2ccc(C(C)(C)C)cc12] I-1084 |
| 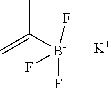 [395083-14-4] | 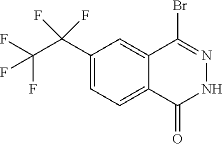 [SMILES: O=c1[nH]nc(Br)c2cc(C(F)(F)C(F)(F)F)ccc12] I-1047 | 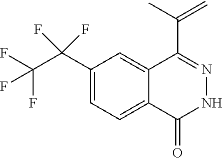 [SMILES: C=C(C)c1n[nH]c(=O)c2ccc(C(F)(F)C(F)(F)F)cc12] I-1085 |
| 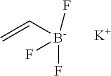 [13682-77-4] | 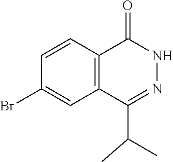 | 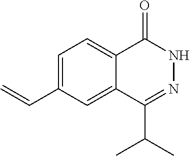 [SMILES: C=Cc1ccc2c(=O)[nH]nc(C(C)C)c2c1] |
| | (I-15) | I-1072 |

### Synthesis of ethyl 2-(1-oxo-6-(trifluoromethyl)-4-(3,3,3-trifluoroprop-1-en-2-yl)phthalazin-2(1H)-yl)acetate (I-129)

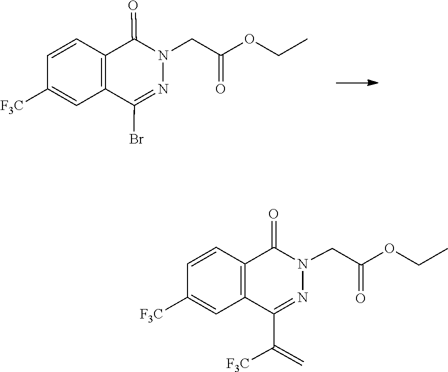 [SMILES: CCOC(=O)Cn1nc(Br)c2cc(C(F)(F)F)ccc2c1=O ; C=C(c1nn(CC(=O)OCC)c(=O)c2ccc(C(F)(F)F)cc12)C(F)(F)F]

Pd(PPh₃)₄[14221-01-3] (76.2 mg, 0.066 mmol) was added to a stirred solution of ethyl 2-(4-bromo-1-oxo-6-(trifluoromethyl)phthalazin-2(1H)-yl)acetate I-81, 1-(trifluoromethyl)vinylboronic acid hexylene glycol ester [1011460-68-6] (307.4 mg, 1.38 mmol) and Na₂CO₃ [497-19-8] (559.1 mg, 5.28 mmol) in DME/DI water (7.6 mL/3.8 mL) at rt under nitrogen. The resulting mixture was stirred at 95° C. for 3.5 hours. The reaction was allowed to cool to rt, diluted with water and extracted with EtOAc. The organic extracts were washed with brine, dried over MgSO₄, filtered and concentrated under vacuum. The crude product was purified by FCC (Hept/EtOAc 0 to 10%) to yield I-129 (287 mg, 52%) as a colorless oil.

### Synthesis of eth 12-(4-ethyl-6-isopropyl-1-oxo-phthalazin-2-yl)acetate (I-35)

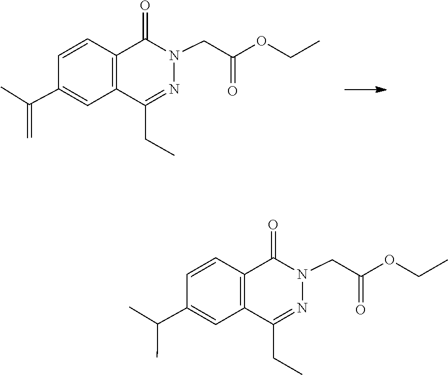

Palladium on active carbon (10%) [7440-05-3] (31 mg) was added to a solution of ethyl 2-(4-ethyl-6-isopropenyl-1-oxo-phthalazin-2-yl)acetate (I-33) (309 mg, 1.03 mmol) in EtOH (13 mL) at 0° C. and under nitrogen. The resulting mixture was stirred under an atmosphere of hydrogen for 16 h. The reaction mixture was filtered through celite, washed with MeOH and the solvents were concentrated in vacuo to yield ethyl 2-(4-ethyl-6-isopropyl-1-oxo-phthalazin-2-yl)acetate (I-35) (300 mg, 95%) as a white solid, that was used in next step without further purification.

Structural analogs were synthesized using the same procedure.

| Starting material | Intermediate |
|---|---|
| 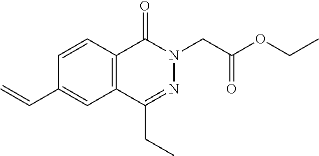 [SMILES: C=Cc1ccc2c(=O)n(CC(=O)OCC)nc(CC)c2c1] | 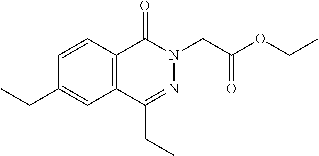 [SMILES: CCOC(=O)Cn1nc(CC)c2cc(CC)ccc2c1=O] |
| (I-34) | (I-36) |
| 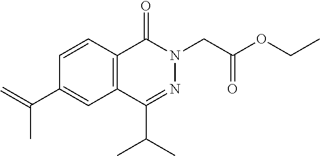 [SMILES: C=C(C)c1ccc2c(=O)n(CC(=O)OCC)nc(C(C)C)c2c1] | 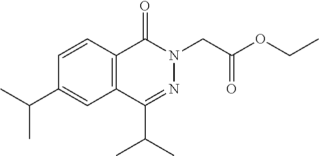 [SMILES: CCOC(=O)Cn1nc(C(C)C)c2cc(C(C)C)ccc2c1=O] |
| (I-43) | (I-45) |
| 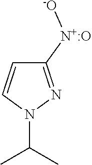 | 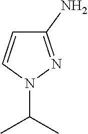 I-1087 |
| I-1086 | |
| 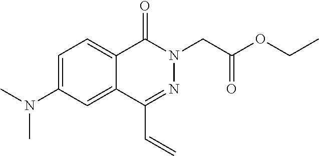 [SMILES: C=Cc1nn(CC(=O)OCC)c(=O)c2ccc(N(C)C)cc12] | 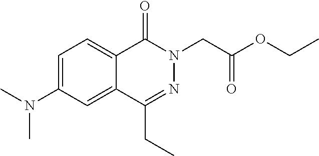 [SMILES: CCOC(=O)Cn1nc(CC)c2cc(N(C)C)ccc2c1=O] |
| I-1082 | I-1088 |
| 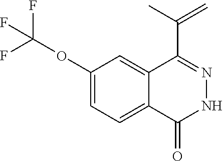 [SMILES: C=C(C)c1n[nH]c(=O)c2ccc(OC(F)(F)F)cc12] | 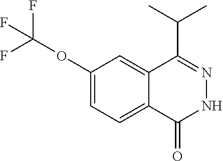 [SMILES: CC(C)c1n[nH]c(=O)c2ccc(OC(F)(F)F)cc12] |
| I-1083 | I-1060 |
| 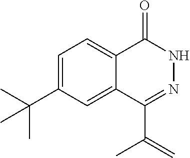 [SMILES: C=C(C)c1n[nH]c(=O)c2ccc(C(C)(C)C)cc12] | 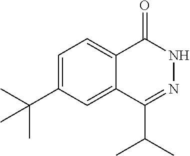 [SMILES: CC(C)c1n[nH]c(=O)c2ccc(C(C)(C)C)cc12] |
| I-1084 | I-1065 |
| 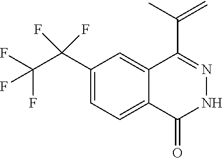 [SMILES: C=C(C)c1n[nH]c(=O)c2ccc(C(F)(F)C(F)(F)F)cc12] | 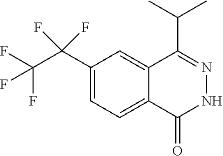 [SMILES: CC(C)c1n[nH]c(=O)c2ccc(C(F)(F)C(F)(F)F)cc12] |
| I-1085 | I-1067 |
| 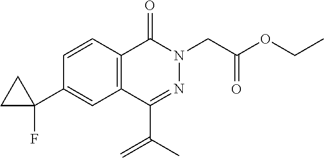 [SMILES: C=C(C)c1nn(CC(=O)OCC)c(=O)c2ccc(C3(F)CC3)cc12] | 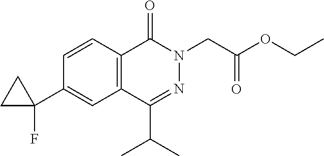 [SMILES: CCOC(=O)Cn1nc(C(C)C)c2cc(C3(F)CC3)ccc2c1=O] |
| I-1089 | I-1090 |

### Synthesis of ethyl 2-(4-ethyl-6-formyl-1-oxo-phthalazin-2-yl)acetate (I-37)

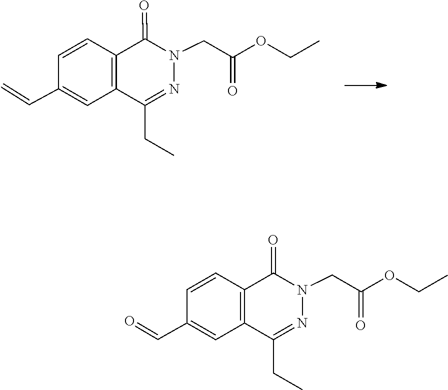 [SMILES: C=Cc1ccc2c(=O)n(CC(=O)OCC)nc(CC)c2c1 ; CCOC(=O)Cn1nc(CC)c2cc(C=O)ccc2c1=O]

Osmium tetroxide [20816-12-0] (2.61 mL, 0.1 mmol) was added to a stirred solution of ethyl 2-(4-ethyl-1-oxo-6-vinyl-phthalazin-2-yl)acetate (1-34) (0.74 g, 2.57 mmol) and sodium periodate [7790-28-5] (1.1 mL, 5.13 mmol) in a mixture of THF (10 mL) and water (10 mL). The mixture was stirred at room temperature for 16 h. The mixture was diluted with water and extracted with EtOAc. The organic layer was separated, dried (MgSO₄), filtered and the solvents evaporated in vacuo. The crude product was purified by flash column chromatography (silica; EtOAc in heptane 0/100 to 15/85). The desired fractions were collected and the solvents evaporated in vacuo to yield (I-37) (0.5 g, 68%) as a white solid.

### Synthesis of ethyl 2-(4-formyl-1-oxo-6-(trifluoromethyl)phthalazin-2(1H)-yl)acetate (I-119)

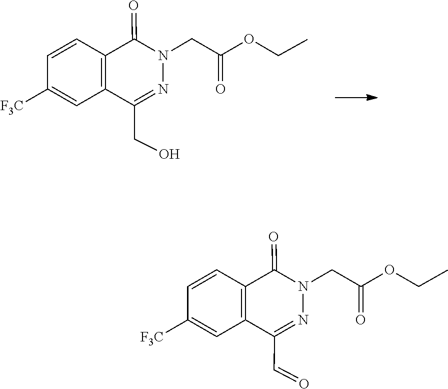 [SMILES: CCOC(=O)Cn1nc(CO)c2cc(C(F)(F)F)ccc2c1=O ; CCOC(=O)Cn1nc(C=O)c2cc(C(F)(F)F)ccc2c1=O]

Dess-Martin periodinane [87413-09-0] (1.1 g, 2.51 mmol) was added portionwise to a stirred solution of ethyl 2-(4-(hydroxymethyl)-1-oxo-6-(trifluoromethyl)phthalazin-2(1H)-yl)acetate I-118 (645 mg, 1.93 mmol) in dry DCM (19.4 mL) at 0° C. The mixture was stirred at rt for 1 hour. The reaction was diluted with a saturated aqueous solution of NaHCO₃ (64 mL) and 10% aqueous Na₂S₂O₃ (10 mL) and extracted with DCM. The organic layer was separated, dried (MgSO₄), filtered and the volátiles concentrated in vacuo. The crude product was purified by FCC (Hept/EtOAc 0 to 42%) to yield I-119 (381 mg, 92% purity, 55%) as a white solid.

### Synthesis of ethyl 2-[6-(difluoromethyl)-4-ethyl-1-oxo-phthalazin-2-yl]acetate (I-38)

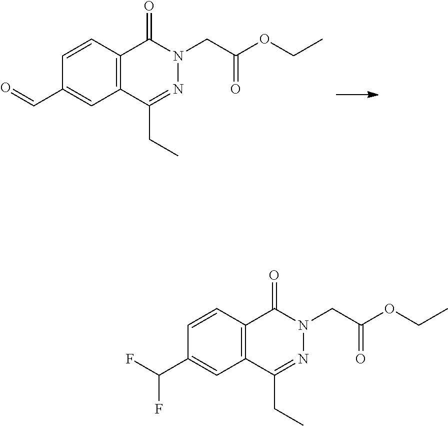 [SMILES: CCOC(=O)Cn1nc(CC)c2cc(C=O)ccc2c1=O ; CCOC(=O)Cn1nc(CC)c2cc(C(F)F)ccc2c1=O]

DAST [38078-09-0] (0.7 mL, 5.27 mmol) was added dropwise to a stirred solution of ethyl 2-(4-ethyl-6-formyl-1-oxo-phthalazin-2-yl)acetate (I-37) (0.5 g, 1.76 mmol) in anhydrous DCM (10 mL) at −10° C. and under nitrogen. The mixture was stirred at room temperature for 16 h. The mixture was diluted with aqueous saturated solution of NaHCO₃ and extracted with DCM. The organic layer was separated, dried (MgSO₄), filtered and the solvents evaporated in vacuo. The crude product was purified by flash column chromatography (silica; EtOAc in heptane 0/100 to 15/85). The desired fractions were collected and the solvents evaporated in vacuo to yield (I-38) (0.39 g, 71%) as a white solid.

Structural analogues were synthesized using the same procedure.

| Starting material | Intermediate |
|---|---|
| 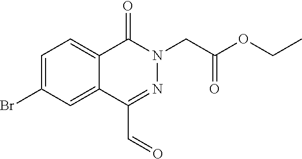 [SMILES: CCOC(=O)Cn1nc(C=O)c2cc(Br)ccc2c1=O] I-1032 | 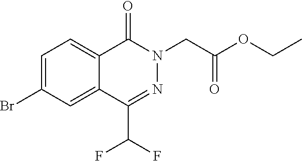 [SMILES: CCOC(=O)Cn1nc(C(F)F)c2cc(Br)ccc2c1=O] I-1091 |
| 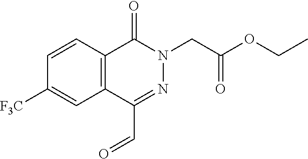 [SMILES: CCOC(=O)Cn1nc(C=O)c2cc(C(F)(F)F)ccc2c1=O] I-119 | 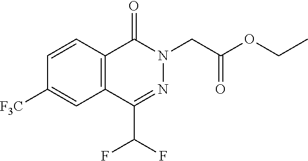 [SMILES: CCOC(=O)Cn1nc(C(F)F)c2cc(C(F)(F)F)ccc2c1=O] I-120 |

### Synthesis of ethyl 2-(1-oxo-4-(2,2,2-trifluoro-1-hydroxyethyl)-6-(trifluoromethyl)phthalazin-2(1H)-yl)acetate (I-125)

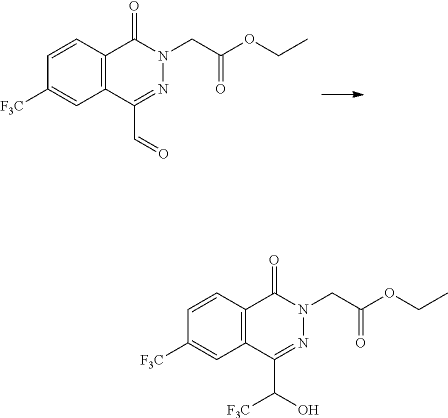 [SMILES: CCOC(=O)Cn1nc(C=O)c2cc(C(F)(F)F)ccc2c1=O ; CCOC(=O)Cn1nc(C(O)C(F)(F)F)c2cc(C(F)(F)F)ccc2c1=O]

A 1M solution of TBAF in THF [429-41-4] (152 μL, 015 mmol) was added dropwise to a stirred solution of ethyl 2-(4-formyl-1-oxo-6-(trifluoromethyl)phthalazin-2(1H)-yl)acetate I-119 (250 mg, 0.76 mmol) and 2-trifluoromethyltrimethylsilane [81290-20-2] (230 μL, 1.52 mmol) in dry THF (6.25 mL) at 0° C. under nitrogen. The reaction was stirred at 0° C. for 15 min, then at rt for 12 hours. The mixture was cooled to 0° C. and a 1M solution of TBAF in THF [429-41-4] (1.5 mL, 1.5 mmol) was added and the mixture was stirred at rt for 20 min. The mixture was diluted with a saturated aqueous solution of NaHCO₃ and extracted with EtOAc. The organic layer was separated, dried (MgSO₄), filtered and the volatiles evaporated in vacuo. The crude product was purified by FCC (Hept/EtOAcO0 to 30%) to yield I-125 (235 mg, 75%) as a white foamy solid.

### Synthesis of ethyl 2-(6-bromo-4-(1-methoxyethyl)-1-oxophthalazin-2(1H)-yl)acetate I-1092

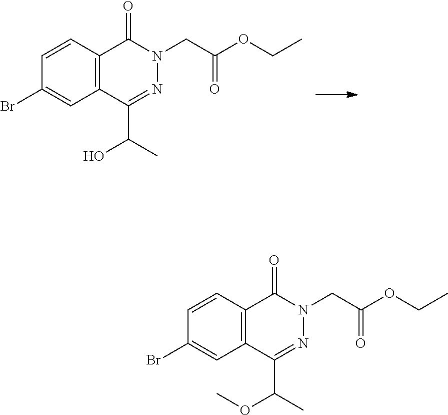 [SMILES: CCOC(=O)Cn1nc(C(C)O)c2cc(Br)ccc2c1=O ; CCOC(=O)Cn1nc(C(C)OC)c2cc(Br)ccc2c1=O]

Trimethyloxonium tetrafluoroborate [420-37-1] (161 mg, 1.09 mmol) and 2,6-di-tert-butyl-4-methylpyridine [38222-83-2] (298 mg, 1.45 mmol) were added in a MW vial, sealed and left under nitrogen (3 vacuum/nitrogen cycles). Then, a suspension of ethyl 2-(6-bromo-4-(1-hydroxyethyl)-1-oxophthalazin-2(1H)-yl)acetate I-1093 (129 mg, 0.36 mmol) in dry dichloromethane (14 mL) was added, and the reaction mixture was stirred at r.t for 1.5 hours. Then, the reaction was quenched by addition of aq. sat. NaHCO₃ and extracted with DCM (×3). The organic layer was separated, dried (MgSO4), filtered and the solvents evaporated in vacuo. The crude product was purified by flash column chromatography (silica, AcOEt in Heptane 0/100 to 50/50). The desired fractions were collected and concentrated in vacuo to yield ethyl 2-(6-bromo-4-(1-methoxyethyl)-1-oxophthalazin-2(1H)-yl)acetate I-1092 (61 mg, 43%) as a yellow oil.

Structural analogs were synthesized using the same procedure.

| Starting material | Intermediate |
|---|---|
| 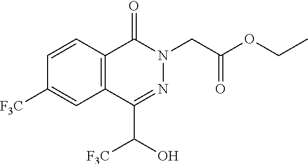 [SMILES: CCOC(=O)Cn1nc(C(O)C(F)(F)F)c2cc(C(F)(F)F)ccc2c1=O] I-124 | 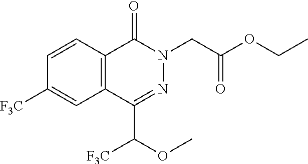 [SMILES: CCOC(=O)Cn1nc(C(OC)C(F)(F)F)c2cc(C(F)(F)F)ccc2c1=O] I-123 |
| 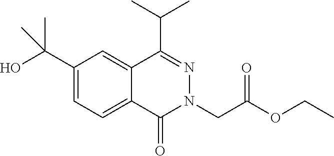 [SMILES: CCOC(=O)Cn1nc(C(C)C)c2cc(C(C)(C)O)ccc2c1=O] I-1064 | I-1094 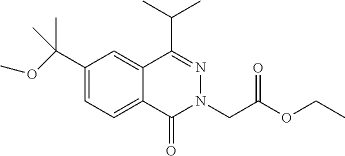 [SMILES: CCOC(=O)Cn1nc(C(C)C)c2cc(C(C)(C)OC)ccc2c1=O] |

### Synthesis of ethyl 2-(6-(dimethylamino)-4-isopropyl-1-oxophthalazin-2(1H)-yl)acetate I-1095

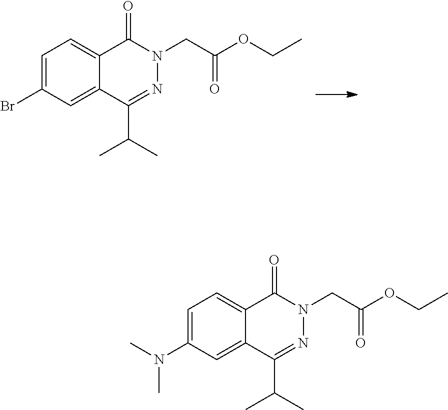

Dimethylamine solution 2M in THF [124-40-3] (0.64 mL, 1.23 mmol) and cesium carbonate [534-17-8] (1107 mg, 3.4 mmol) were added to a stirred solution of ethyl 2-(6-bromo-4-isopropyl-1-oxophthalazin-2(1H)-yl)acetate (1-31) (300 mg, 0.85 mmol) in 1,4-dioxane (10 mL) in a sealed tube. The mixture was bubbled with N2 for 10 min. RuPhos Pd G4 [1599466-85-9] (145 mg, 0.17 mmol) was added to the mixture and the reaction was stirred at 70° C. for 16 h. Then, dimethylamine solution 2M in THF [124-40-3] (0.64 mL, 1.23 mmol), cesium carbonate [534-17-8] (277 mg, 0.85 mmol) and RuPhos Pd G4 [1599466-85-9] (145 mg, 0.17 mmol) were added and the mixture was stirred at 75° C. for 16. The mixture was diluted with brine and extracted with EtOAc. The organic layer was washed with brine, dried (MgSO4), filtered and the solvents evaporated in vacuo. The crude product was purified by flash column chromatography (silica; EtOAc in heptane 0/100 to 50/50). The desired fractions were collected and concentrated in vacuo to yield ethyl 2-(6-(dimethylamino)-4-isopropyl-1-oxophthalazin-2(1H)-yl)acetate I-1095 (208 mg, 73%) as a brown oil.

### Synthesis of ethyl 2-(4-(sec-butyl)-1-oxo-6-(trifluoromethyl)phthalazin-2(1H)-yl)acetate I-102

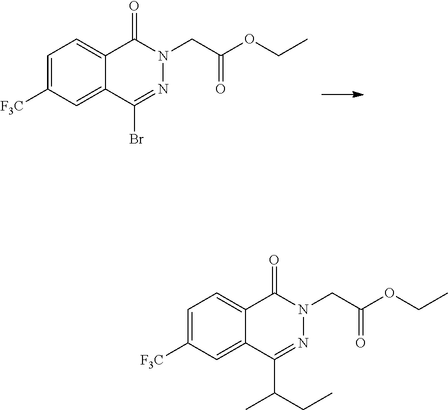 [SMILES: CCOC(=O)Cn1nc(Br)c2cc(C(F)(F)F)ccc2c1=O ; CCOC(=O)Cn1nc(C(C)CC)c2cc(C(F)(F)F)ccc2c1=O]

A 0.5M solution of sec-butylzinc bromide [171860-66-5] (12.7 mL, 6.33 mmol) was added dropwise to a stirred solution of ethyl 2-(4-bromo-1-oxo-6-(trifluoromethyl)phthalazin-2(1H)-yl)acetate I-81 (0.8 g, 2.11 mmol) and bis(tri-tert-butylphosphine)palladium(0) [53199-31-8] (107.8 mg, 0.21 mmol) in dry THF (20 mL). The resulting mixture was stirred at 40° C. for 6 hours. The reaction mixture was quenched by addition of a saturated aqueous solution of NH₄Cl. The volatiles were removed under vacuum and the aqeuous phase was extracted with EtOAc. The organic extracts were washed with brine and dried over MgSO₄. The crude compound was purified by FCC (Hept/EtOAc 0/100 to 25/75) to yield a white solid which was further purified by preparative SFC (Stationary phase: Chiralpak Daicel IC 20×250 mm, Mobile phase: CO₂, EtOH+0.4 iPrNH₂) yielding I-102 (65 mg, 9%) as a white solid.

Structural analogs were synthesized using the same procedure.

| Reagent | Starting material | Intermediate |
|---|---|---|
| 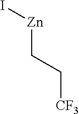 [1174507-16-4] | 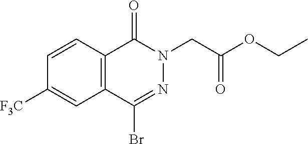 [SMILES: CCOC(=O)Cn1nc(Br)c2cc(C(F)(F)F)ccc2c1=O] (I-81) | 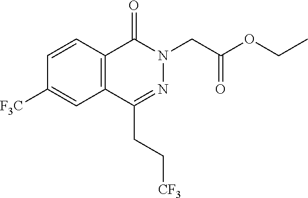 [SMILES: CCOC(=O)Cn1nc(CCC(F)(F)F)c2cc(C(F)(F)F)ccc2c1=O] (I-106) |
| 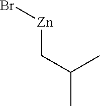 [126403-67-6] | 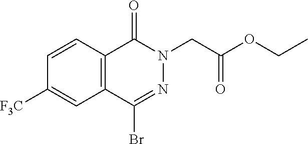 [SMILES: CCOC(=O)Cn1nc(Br)c2cc(C(F)(F)F)ccc2c1=O] (I-81) | 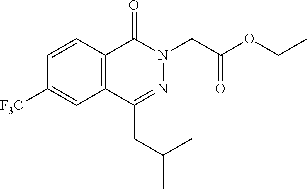 [SMILES: CCOC(=O)Cn1nc(CC(C)C)c2cc(C(F)(F)F)ccc2c1=O] (I-107) |
| 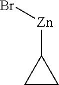 [126403-68-7] | 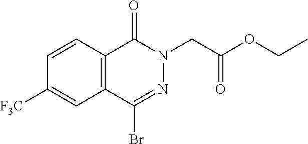 [SMILES: CCOC(=O)Cn1nc(Br)c2cc(C(F)(F)F)ccc2c1=O] (I-81) | 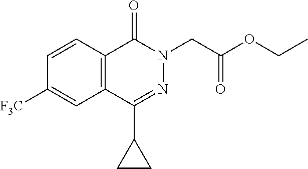 [SMILES: CCOC(=O)Cn1nc(C2CC2)c2cc(C(F)(F)F)ccc2c1=O] (I-130) |

### Synthesis of ethyl 2-(8-iodo-4-isopropyl-1-oxo-6-(trifluoromethyl)phthalazin-2(1H)-yl)acetate I-84

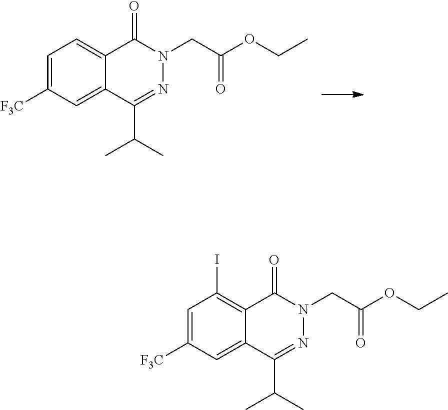 [SMILES: CCOC(=O)Cn1nc(C(C)C)c2cc(C(F)(F)F)ccc2c1=O ; CCOC(=O)Cn1nc(C(C)C)c2cc(C(F)(F)F)cc(I)c2c1=O]

Ethyl 2-(4-isopropyl-1-oxo-6-(trifluoromethyl)phthalazin-2(1H)-yl)acetate (1-29) (600 mg, 1.74 mmol), [Cp*Rh(MeCN)₃](SbF₆)₂[59738-27-1] (144.5 mg, 0.17 mmol), NIS [516-12-1] (780.8 mg, 3.47 mmol) and NaOAc [127-09-3] (28.5 mg, 0.35 mmol) were placed in a 20-mL MW vial. The vial was sealed and dry 1,2-DCE (10 mL) was added and suspension was then heated at 120° C. for 16 hours. The crude mixture was diluted with DCM (30 mL) and quenched by addition of a saturated aqueous solution of Na₂S₂O₃ (50 mL). After stirring the biphasic mixture vigorously at rt for 5 min, the 2 layers were separated and the aqueous layer back-extracted with DCM (2×20 mL). the combined organic extracts were dried over Na₂SO₄, filtered and concentrated in vacuo. The obtained residue was purified by FCC (Hept/EtOAc 93:7 to 7:3) to afford I-84 (360 mg, 44%) as a pale pink solid.

### Synthesis of ethyl 2-(8-hydroxy-4-isopropyl-1-oxo-6-(trifluoromethyl)phthalazin-2(1H)-yl)acetate I-74

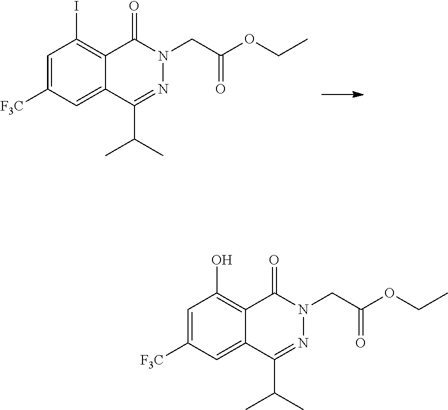 [SMILES: CCOC(=O)Cn1nc(C(C)C)c2cc(C(F)(F)F)cc(I)c2c1=O ; CCOC(=O)Cn1nc(C(C)C)c2cc(C(F)(F)F)cc(O)c2c1=O]

Ethyl 2-(8-iodo-4-isopropyl-1-oxo-6-(trifluoromethyl)phthalazin-2(1H)-yl)acetate (I-74) (200 mg, 0.43 mmol), PdCl₂(dppf) [72287-26-4] (31.3 mg, 0.043 mmol), KOAc [127-08-2] (125.8 mg, 1.28 mmol) and bis(pinacolato)diboron [73183-34-3] (217 mg, 0.85 mmol) were placed in a 20-mL MW vial. The vial was sealed and placed under nitrogen (3 vacuum/nitrogen cycles) and dry DMSO (4 mL) was added. The suspension was then heated at 80° C. for 16 hours. The crude mixture was diluted with brine (30 mL) and extracted with DCM (4×25 mL). The combined organic layers were dried over Na₂SO₄, filtered and concentrated in vacuo. The obtained dark brown residue (crude ethyl 2-(4-isopropyl-1-oxo-8-(4,4,5,5-tetramethyl-1,3,2-dioxaborolan-2-yl)-6-(trifluoromethyl)phthalazin-2(1H)-yl)acetate) and sodium perborate tetrahydrate (265 mg, 1.72 mmol) were placed in a screw-cap tube and a 1:1 mixture of THF/DI water (2 mL) was added and the mixture stirred vigorously at rt for 2 hours. A further portion of sodium perborate tetrahydrate (265 mg, 1.72 mmol, 4 equiv) was added and the mixture stirred for further 14 hours at rt.

The mixture was diluted with DI water (5 mL), the pH acidified to pH<3 and the mixture extracted with DCM (3×15 mL). The combined organic extracts were dried over Na₂SO₄, filtered and concentrated in vacuo. The resulting brown residue was purified by FCC (Hept/EtOAc 95:5 to 4:1) to afford I-74 (85.5 mg, 55%) as a colorless crystalline solid.

### Synthesis of ethyl 2-(4-(2-fluoropropan-2-yl)-1-oxo-6-(trifluoromethyl)phthalazin-2(1H)-yl)acetate I-112

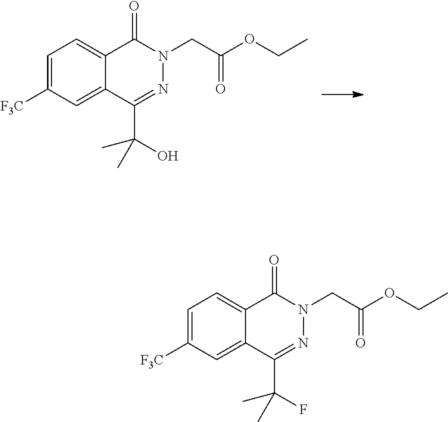 [SMILES: CCOC(=O)Cn1nc(C(C)(C)O)c2cc(C(F)(F)F)ccc2c1=O ; CCOC(=O)Cn1nc(C(C)(C)F)c2cc(C(F)(F)F)ccc2c1=O]

DAST [38078-09-0] (394 μL, 2.84 mmol) was added dropwise to a stirred solution of ethyl 2-(4-(2-hydroxypropan-2-yl)-1-oxo-6-(trifluoromethyl)phthalazin-2(1H)-yl)acetate I-113 (508 mg, 1.42 mmol) in dry DCM (13.7 mL) at −78° C. and under nitrogen. The reaction mixture allowed to warm from −78° C. to 0° C. over 1 hour and stirred at rt for 16 hours. The mixture was diluted with a saturated aqueous solution of NaHCO₃ and extracted with EtOAc. The organic layer was separated, dried (MgSO4), filtered and the solvent evaporated in vacuo. The crude product was purified by FCC (Hept/EtOAc 0 to 16%). The desired fractions were collected and concentrated in vacuo to yield I-112 (458 mg, 89%) as a white solid.

Structural analogs were synthesized using the same procedure.

| Intermediate | Product |
|---|---|
| 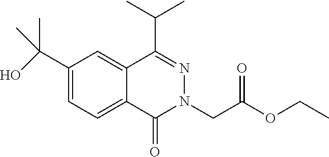 [SMILES: CCOC(=O)Cn1nc(C(C)C)c2cc(C(C)(C)O)ccc2c1=O] I-1064 | 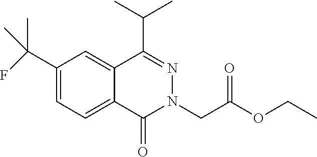 [SMILES: CCOC(=O)Cn1nc(C(C)C)c2cc(C(C)(C)F)ccc2c1=O] I-1096 |

### Synthesis of ethyl 2-(6-cyclobutyl-4-isopropyl-1-oxophthalazin-2(1H)-yl)acetate I-1097

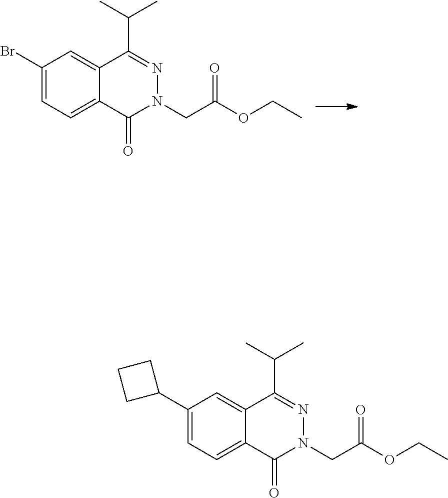 [SMILES: CCOC(=O)Cn1nc(C(C)C)c2cc(Br)ccc2c1=O ; CCOC(=O)Cn1nc(C(C)C)c2cc(C3CCC3)ccc2c1=O]

Ethyl 2-(6-bromo-4-isopropyl-1-oxophthalazin-2(1H)-yl)acetate (I-31) (894 mg, 2.531 mmol) and potassium cyclobutyltrifluoroborate [395083-14-4] (451 mg, 2.784 mmol) were added to a stirred solution of Pd(OAc)_{2 [3375}-31-3] (57 mg, 0.253 mmol), cataCXium A [321921-71-5] (91 mg, 0.253 mmol) and cesium carbonate [534-17-8](2.47 g, 7.593 mmol) in toluene (20 mL) and water (2 mL) while N2 is bubbling. The resulting mixture was heated at 100° C. for 16 h. The reaction mixture was diluted with H2O and the organic layer was extracted with DCM, dried with MgSO4 anh., filtered and the solvents evaporated in vacuo. The crude product was purified by flash column chromatography (silica 25 g; EtOAc in heptane 0/100 to 15/85). The desired fractions were collected and concentrated in vacuo to yield ethyl 2-(6-cyclobutyl-4-isopropyl-1-oxophthalazin-2(1H)-yl)acetate I-1097 (176 mg, 21%) as a yellow oil.

### Synthesis of 4-isopropyl-1-oxo-1,2-dihydrophthalazine-6-carbonitrile I-1069

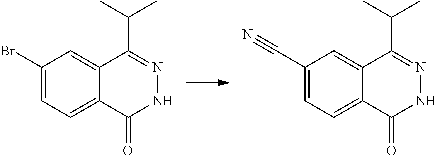 [SMILES: CC(C)c1n[nH]c(=O)c2ccc(Br)cc12 ; CC(C)c1n[nH]c(=O)c2ccc(C#N)cc12]

tBuXPhos [564483-19-8] and Pd2(dba)3 [51364-51-3] were added to DMA while the solvent was degassed by bubbling nitrogen at 45° C. The mixture was stirred under nitrogen at 45° C. for 5 minutes. Zn [7440-66-6] and zinc cyanide [557-21-1] were added under nitrogen at 45° C. 6-Bromo-4-isopropylphthalazin-1(2H)-one (I-15) was added under nitrogen at 45° C. The mixture was stirred in a sealed tube at 120° C. for 16 h. The mixture was cooled down to rt, then was diluted with sat NaHCO₃ and extracted with AcOEt. The organic layer was separated, washed with water, dried (MgSO4), filtered and the solvents evaporated in vacuo. The crude product was purified by flash column chromatography (silica 25 g; AcOEt in heptane 0/100 to 70/30). The desired fractions were collected and concentrated in vacuo to yield 4-isopropyl-1-oxo-1,2-dihydrophthalazine-6-carbonitrile I-1069 (100 mg, 31%) as a white solid.

### Synthesis of ethyl 2-(6-(1,1-difluoroethyl)-4-isopropyl-1-oxophthalazin-2(1H)-yl)acetate I-1098

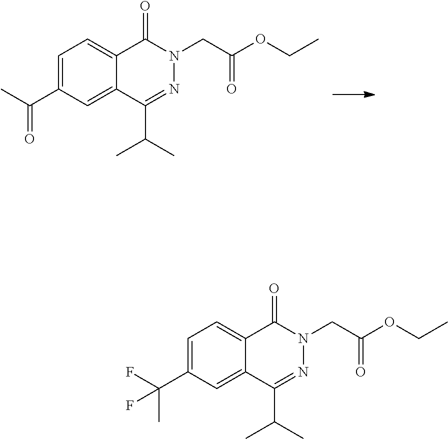 [SMILES: CCOC(=O)Cn1nc(C(C)C)c2cc(C(C)=O)ccc2c1=O ; CCOC(=O)Cn1nc(C(C)C)c2cc(C(C)(F)F)ccc2c1=O]

To a mixture of ethyl 2-(6-acetyl-4-isopropyl-1-oxophthalazin-2(1H)-yl)acetate I-1071 (0.3 g, 0.94 mmol) in DCM (8 ml), diethylaminosulfur trifluoride [38078-09-0] (1 mL, 1.22 g/mL, 7.56 mmol) and triethylamine trihydrofluoride [73602-61-6] (0.47 mL, 0.989 g/mL, 2.85 mmol) were added at rt. The mixture was stirred at 50° C. for two days. The crude was cooled at 0° C. and quenched with a saturated solution of NaHCO₃ (dropwise). The crude was extracted with CH2Cl2 (2×5 ml), the organic layers were dried and evaporated in vacuo to afford an oil which was purified by column chromatography (SiO2, CH2Cl2). The desired fractions were concentrated to yield ethyl 2-(6-(1,1-difluoroethyl)-4-isopropyl-1-oxophthalazin-2(1H)-yl)acetate I-1098 (189 mg, yield 59%) as oil.

### Synthesis of ethyl 2-(6-(2,2-difluorocyclopropyl)-4-isopropyl-1-oxophthalazin-2(1H)-yl)acetate I-1099

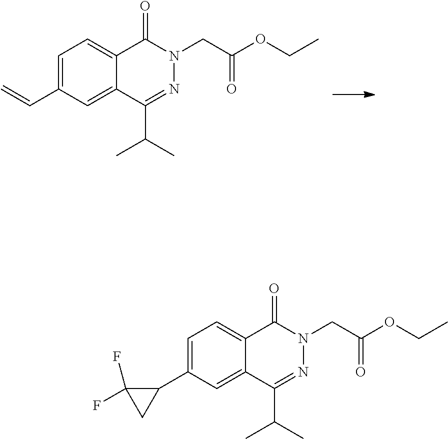 [SMILES: C=Cc1ccc2c(=O)n(CC(=O)OCC)nc(C(C)C)c2c1 ; CCOC(=O)Cn1nc(C(C)C)c2cc(C3CC3(F)F)ccc2c1=O]

Methyl fluorosulfonyldifluoroacetate [680-15-9] (0.99 mL, 1.52 g/mL, 7.84 mmol) was added to a stirred solution of ethyl 2-(4-isopropyl-1-oxo-6-vinylphthalazin-2(1H)-yl)acetate I-1073 (589 mg, 1.96 mmol) and potassium iodide [7681-11-0] (1.3 g, 7.84 mmol) in propionitrile (6 mL) at rt. The mixture was stirred in a sealed tube at 50° C. for 120 h. After cooling to rt, the mixture was quenched water and extracted with heptane (3×). The organic layers were separated, combined, wash with saturated aq NaHCO₃ and brine, dried (MgSO4), filtered and the solvents evaporated in vacuo. The crude product was purified by flash column chromatography (silica 25 g; EtOAc in heptane 0/100 to 40/60). The desired fractions were collected and concentrated in vacuo to yield ethyl 2-(6-(2,2-difluorocyclopropyl)-4-isopropyl-1-oxophthalazin-2(1H)-yl)acetate I-1099 (200 mg, 29%) as yellow oil.

### Synthesis of 2-(6-ethyl-4-isopropyl-1-oxophthalazin-2(1H)-yl)acetic acid I-1100

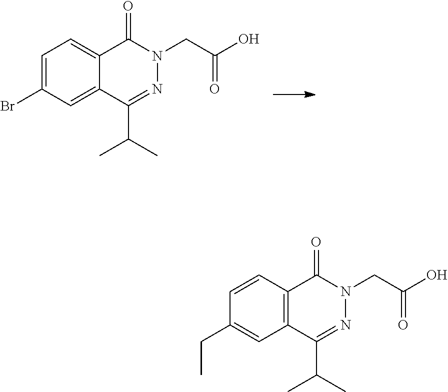 [SMILES: CC(C)c1nn(CC(=O)O)c(=O)c2ccc(Br)cc12 ; CCc1ccc2c(=O)n(CC(=O)O)nc(C(C)C)c2c1]

To a mixture of 2-(6-bromo-4-isopropyl-1-oxophthalazin-2(1H)-yl)acetic acid (I-53) (250 mg, 0.7688 mmol) in THF (7 ml), triethylborane [97-94-9] (2.3 mL, 1 M, 2.306 mmol), Cs2CO3 [534-17-8] (751.5 mg, 2.31 mmol) and [1,1′-bis(diphenylphosphino)ferrocene]dichloropalladium(II) [72287-26-4] (56.65 mg, 0.077 mmol) were added, the mixture was bubbled with N2 for 10 min. The reaction was stirred at 95° C. for 16 h. The crude was treated with water and acidified with HCl (2N to PH=2-3), the organic layer was extracted with AcOEt (2×5 ml), the organic layers were dried and evaporated in vacuo to afford 2-(6-ethyl-4-isopropyl-1-oxophthalazin-2(1H)-yl)acetic acid I-1100 (130 mg, yield 62%) as solid.

### Synthesis of 2-(4-isopropyl-6-methoxy-1-oxo-phthalazin-2-yl)acetic acid (I-39)

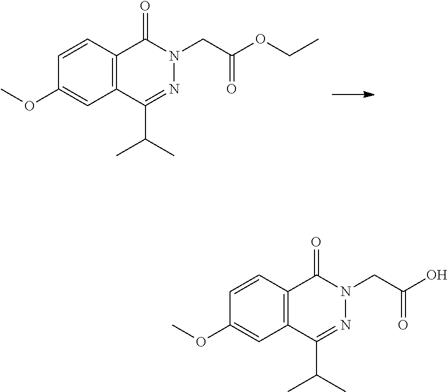

Lithium hydroxide [1310-65-2] (45 mg, 1.88 mmol) was added to a stirred solution of ethyl 2-(4-isopropyl-6-methoxy-1-oxo-phthalazin-2-yl)acetate (I-21) (163 mg, 0.54 mmol) in THF (2 mL) and water (0.5 mL). The mixture was stirred at room temperature for 2 h. The solvent was partially removed in vacuo and the residue was diluted with water and acidified with 1N aqueous solution of HCl until pH=5. The solid formed was filtered and dried under vacuo at 50° C. for 2 h to yield 2-(4-isopropyl-6-methoxy-1-oxo-phthalazin-2-yl)acetic acid (I-39) (116 mg, 78%) as white solid, that was used in next step without further purification.

Structural analogs were synthesized using the same procedure.

| Starting material | Intermediate |
|---|---|
| 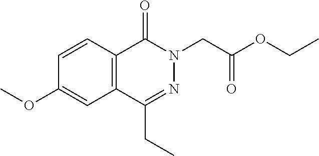 [SMILES: CCOC(=O)Cn1nc(CC)c2cc(OC)ccc2c1=O] (I-22) | 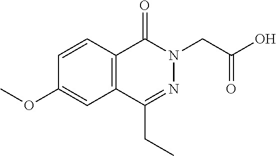 [SMILES: CCc1nn(CC(=O)O)c(=O)c2ccc(OC)cc12] (I-40) |
| 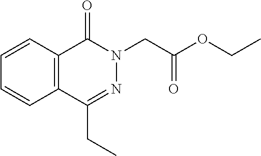 [SMILES: CCOC(=O)Cn1nc(CC)c2ccccc2c1=O] (I-23) | 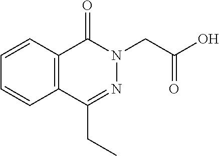 [SMILES: CCc1nn(CC(=O)O)c(=O)c2ccccc12] (I-41) |
| 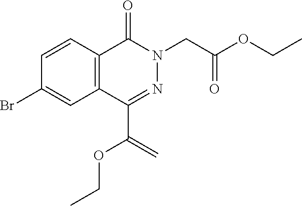 [SMILES: C=C(OCC)c1nn(CC(=O)OCC)c(=O)c2ccc(Br)cc12] I-1074 | 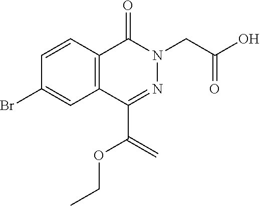 [SMILES: C=C(OCC)c1nn(CC(=O)O)c(=O)c2ccc(Br)cc12] I-1101 |
| 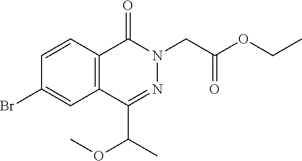 [SMILES: CCOC(=O)Cn1nc(C(C)OC)c2cc(Br)ccc2c1=O] I-1092 | 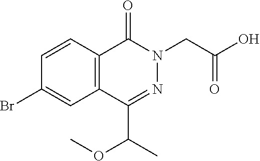 [SMILES: COC(C)c1nn(CC(=O)O)c(=O)c2ccc(Br)cc12] I-1102 |
| 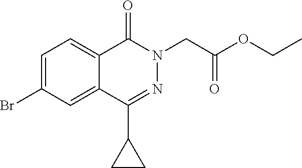 [SMILES: CCOC(=O)Cn1nc(C2CC2)c2cc(Br)ccc2c1=O] I-1057 | 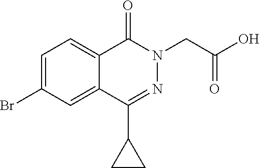 [SMILES: O=C(O)Cn1nc(C2CC2)c2cc(Br)ccc2c1=O] I-1103 |
| 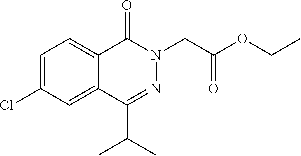 [SMILES: CCOC(=O)Cn1nc(C(C)C)c2cc(Cl)ccc2c1=O] I-1058 | 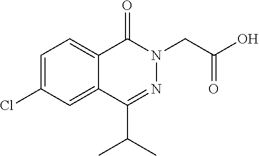 [SMILES: CC(C)c1nn(CC(=O)O)c(=O)c2ccc(Cl)cc12] I-1104 |
| 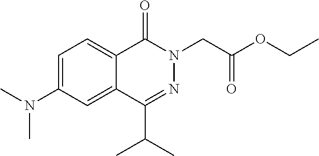 [SMILES: CCOC(=O)Cn1nc(C(C)C)c2cc(N(C)C)ccc2c1=O] I-1095 | 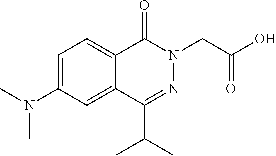 [SMILES: CC(C)c1nn(CC(=O)O)c(=O)c2ccc(N(C)C)cc12] I-1105 |
| 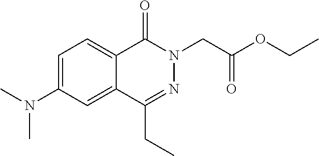 [SMILES: CCOC(=O)Cn1nc(CC)c2cc(N(C)C)ccc2c1=O] I-1088 | 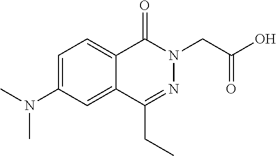 [SMILES: CCc1nn(CC(=O)O)c(=O)c2ccc(N(C)C)cc12] I-1106 |
| 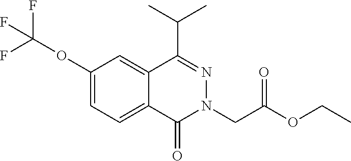 [SMILES: CCOC(=O)Cn1nc(C(C)C)c2cc(OC(F)(F)F)ccc2c1=O] I-1061 | 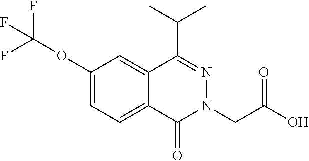 [SMILES: CC(C)c1nn(CC(=O)O)c(=O)c2ccc(OC(F)(F)F)cc12] I-1107 |
| 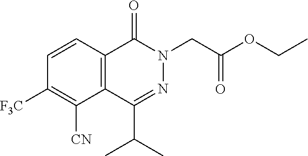 [SMILES: CCOC(=O)Cn1nc(C(C)C)c2c(C#N)c(C(F)(F)F)ccc2c1=O] (I-87) | 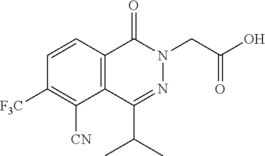 [SMILES: CC(C)c1nn(CC(=O)O)c(=O)c2ccc(C(F)(F)F)c(C#N)c12] (I-73) |
| 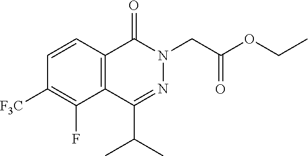 [SMILES: CCOC(=O)Cn1nc(C(C)C)c2c(F)c(C(F)(F)F)ccc2c1=O] (I-88) | 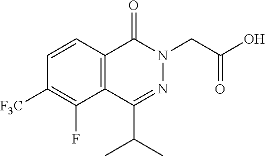 [SMILES: CC(C)c1nn(CC(=O)O)c(=O)c2ccc(C(F)(F)F)c(F)c12] (I-58) |
| 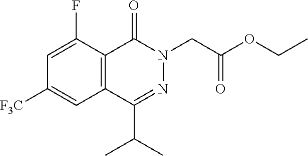 [SMILES: CCOC(=O)Cn1nc(C(C)C)c2cc(C(F)(F)F)cc(F)c2c1=O] (I-101) | 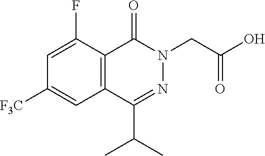 [SMILES: CC(C)c1nn(CC(=O)O)c(=O)c2c(F)cc(C(F)(F)F)cc12] (I-64) |
| 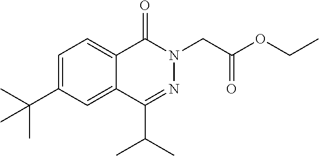 [SMILES: CCOC(=O)Cn1nc(C(C)C)c2cc(C(C)(C)C)ccc2c1=O] I-1066 | 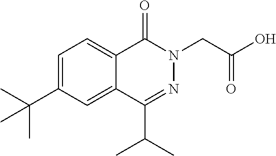 [SMILES: CC(C)c1nn(CC(=O)O)c(=O)c2ccc(C(C)(C)C)cc12] I-1108 |
| 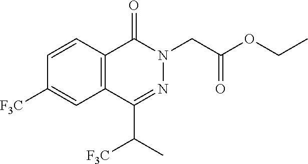 [SMILES: CCOC(=O)Cn1nc(C(C)C(F)(F)F)c2cc(C(F)(F)F)ccc2c1=O] (I-128) | 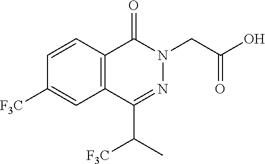 [SMILES: CC(c1nn(CC(=O)O)c(=O)c2ccc(C(F)(F)F)cc12)C(F)(F)F] (I-62) |
| 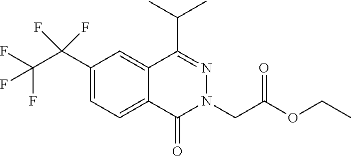 [SMILES: CCOC(=O)Cn1nc(C(C)C)c2cc(C(F)(F)C(F)(F)F)ccc2c1=O] I-1068 | 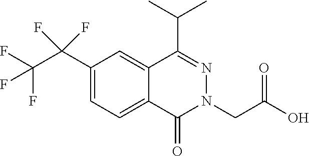 [SMILES: CC(C)c1nn(CC(=O)O)c(=O)c2ccc(C(F)(F)C(F)(F)F)cc12] I-1109 |
| 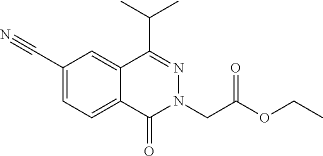 [SMILES: CCOC(=O)Cn1nc(C(C)C)c2cc(C#N)ccc2c1=O] I-1070 | 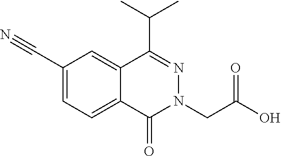 [SMILES: CC(C)c1nn(CC(=O)O)c(=O)c2ccc(C#N)cc12] I-1110 |
| 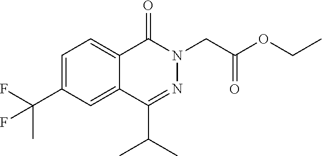 [SMILES: CCOC(=O)Cn1nc(C(C)C)c2cc(C(C)(F)F)ccc2c1=O] I-1098 | 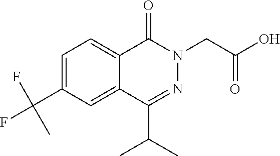 [SMILES: CC(C)c1nn(CC(=O)O)c(=O)c2ccc(C(C)(F)F)cc12] I-1111 |
| 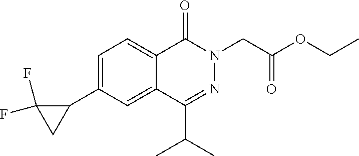 [SMILES: CCOC(=O)Cn1nc(C(C)C)c2cc(C3CC3(F)F)ccc2c1=O] I-1099 | 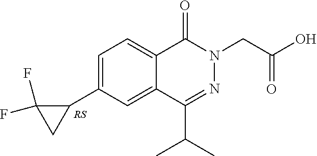 [SMILES: CC(C)c1nn(CC(=O)O)c(=O)c2ccc(C3CC3(F)F)cc12] I-1112 |
| 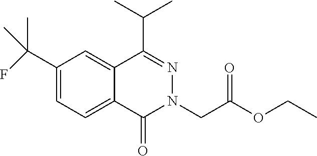 [SMILES: CCOC(=O)Cn1nc(C(C)C)c2cc(C(C)(C)F)ccc2c1=O] I-1096 | 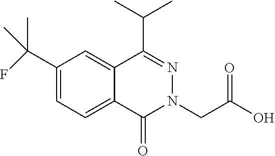 [SMILES: CC(C)c1nn(CC(=O)O)c(=O)c2ccc(C(C)(C)F)cc12] I-1113 |

### Synthesis of 2-(4-ethyl-6-bromo-1-oxo-phthalazin-2-yl)acetic acid (I-44)

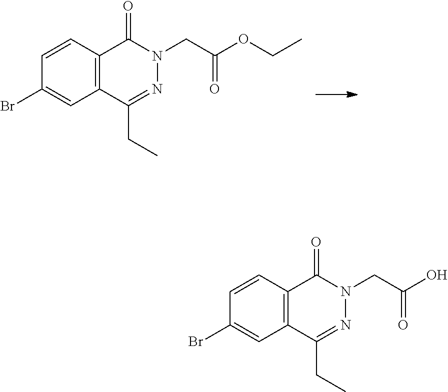 [SMILES: CCOC(=O)Cn1nc(CC)c2cc(Br)ccc2c1=O ; CCc1nn(CC(=O)O)c(=O)c2ccc(Br)cc12]

A 1N aqueous solution of NaOH [1310-73-2] (2 mL, 2 mmol) was added to a stirred solution of ethyl 2-(6-bromo-4-ethyl-1-oxo-phthalazin-2-yl)acetate (1-31) (200 mg, 0.59 mmol) in MeOH (3 mL). The mixture was stirred at 70° C. for 1.5 h. The mixture was acidified with 1N aqueous solution of HCl until pH=1. and then extracted with DCM. The organic layer was separated, dried (Na2SO4), filtered and the solvents evaporated in vacuo to yield 2-(4-ethyl-6-bromo-1-oxo-phthalazin-2-yl)acetic acid (I-44) (150 mg, 82%) as white solid, that was used in next step without further purification.

Note: this reaction can most of the time be performed at rt for 2 hours.

Structural analogues were synthesized using the same procedure.

| Starting Material | Intermediate |
|---|---|
| 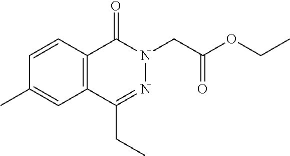 [SMILES: CCOC(=O)Cn1nc(CC)c2cc(C)ccc2c1=O] (I-30) | 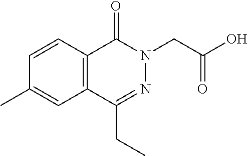 [SMILES: CCc1nn(CC(=O)O)c(=O)c2ccc(C)cc12] (I-46) |
| 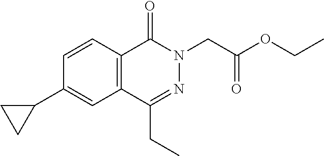 [SMILES: CCOC(=O)Cn1nc(CC)c2cc(C3CC3)ccc2c1=O] (I-32) | 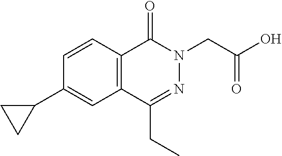 [SMILES: CCc1nn(CC(=O)O)c(=O)c2ccc(C3CC3)cc12] (I-47) |
| 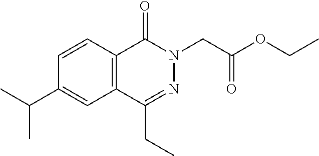 [SMILES: CCOC(=O)Cn1nc(CC)c2cc(C(C)C)ccc2c1=O] (I-35) | 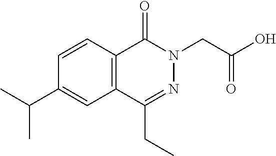 [SMILES: CCc1nn(CC(=O)O)c(=O)c2ccc(C(C)C)cc12] (I-48) |
| 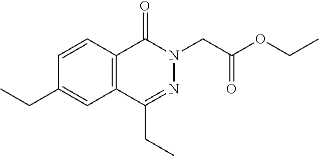 [SMILES: CCOC(=O)Cn1nc(CC)c2cc(CC)ccc2c1=O] (I-36) | 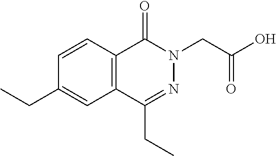 [SMILES: CCc1ccc2c(=O)n(CC(=O)O)nc(CC)c2c1] (I-49) |
| 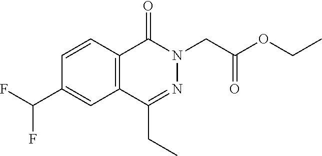 [SMILES: CCOC(=O)Cn1nc(CC)c2cc(C(F)F)ccc2c1=O] (I-38) | 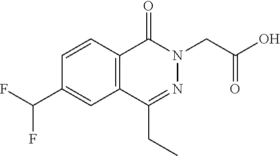 [SMILES: CCc1nn(CC(=O)O)c(=O)c2ccc(C(F)F)cc12] (I-50) |
| 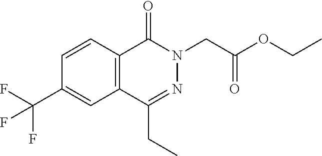 [SMILES: CCOC(=O)Cn1nc(CC)c2cc(C(F)(F)F)ccc2c1=O] (I-25) | 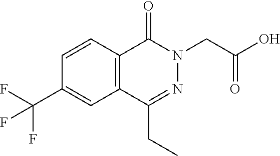 [SMILES: CCc1nn(CC(=O)O)c(=O)c2ccc(C(F)(F)F)cc12] (I-51) |
| 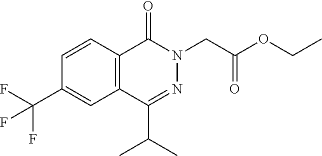 [SMILES: CCOC(=O)Cn1nc(C(C)C)c2cc(C(F)(F)F)ccc2c1=O] (I-29) | 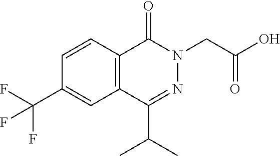 [SMILES: CC(C)c1nn(CC(=O)O)c(=O)c2ccc(C(F)(F)F)cc12] (I-52) |
| 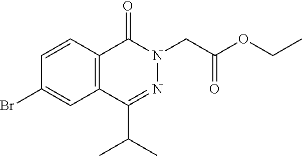 [SMILES: CCOC(=O)Cn1nc(C(C)C)c2cc(Br)ccc2c1=O] (I-31) | 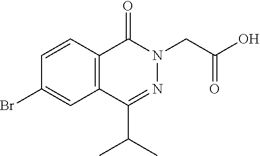 [SMILES: CC(C)c1nn(CC(=O)O)c(=O)c2ccc(Br)cc12] (I-53) |
| 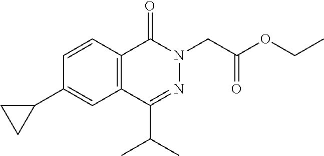 [SMILES: CCOC(=O)Cn1nc(C(C)C)c2cc(C3CC3)ccc2c1=O] (I-56) | 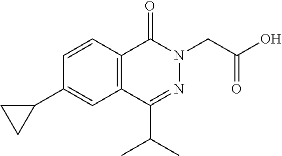 [SMILES: CC(C)c1nn(CC(=O)O)c(=O)c2ccc(C3CC3)cc12] (I-54) |
| 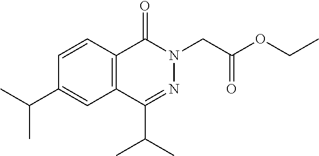 [SMILES: CCOC(=O)Cn1nc(C(C)C)c2cc(C(C)C)ccc2c1=O] (I-45) | 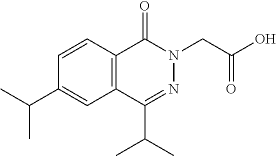 [SMILES: CC(C)c1ccc2c(=O)n(CC(=O)O)nc(C(C)C)c2c1] (I-55) |
| 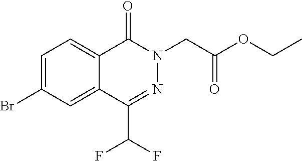 [SMILES: CCOC(=O)Cn1nc(C(F)F)c2cc(Br)ccc2c1=O] I-1091 | 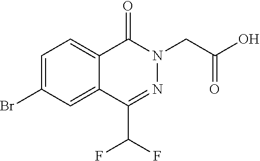 [SMILES: O=C(O)Cn1nc(C(F)F)c2cc(Br)ccc2c1=O] I-1114 |
| 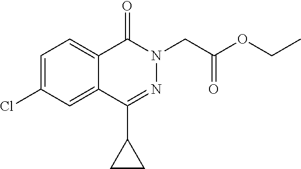 [SMILES: CCOC(=O)Cn1nc(C2CC2)c2cc(Cl)ccc2c1=O] I-1059 | 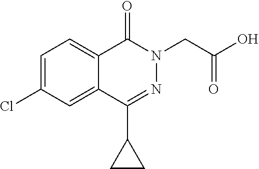 [SMILES: O=C(O)Cn1nc(C2CC2)c2cc(Cl)ccc2c1=O] I-1115 |
| 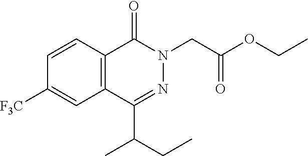 [SMILES: CCOC(=O)Cn1nc(C(C)CC)c2cc(C(F)(F)F)ccc2c1=O] (I-102) | 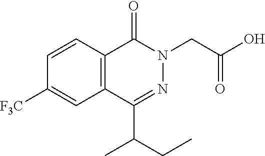 [SMILES: CCC(C)c1nn(CC(=O)O)c(=O)c2ccc(C(F)(F)F)cc12] (I-57) |
| 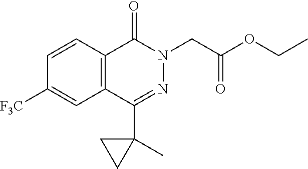 [SMILES: CCOC(=O)Cn1nc(C2(C)CC2)c2cc(C(F)(F)F)ccc2c1=O] (I-104) | 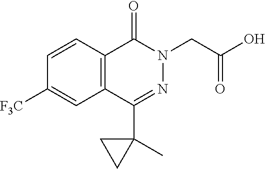 [SMILES: CC1(c2nn(CC(=O)O)c(=O)c3ccc(C(F)(F)F)cc23)CC1] (I-67) |
| 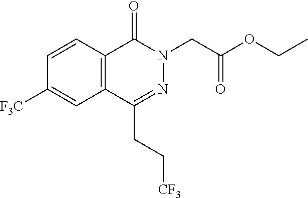 [SMILES: CCOC(=O)Cn1nc(CCC(F)(F)F)c2cc(C(F)(F)F)ccc2c1=O] (I-106) | 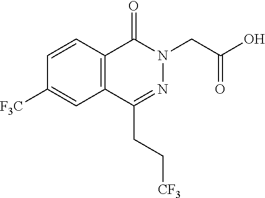 [SMILES: O=C(O)Cn1nc(CCC(F)(F)F)c2cc(C(F)(F)F)ccc2c1=O] (I-65) |
| 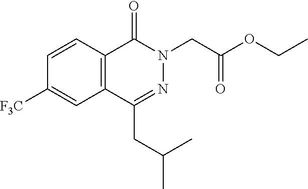 [SMILES: CCOC(=O)Cn1nc(CC(C)C)c2cc(C(F)(F)F)ccc2c1=O] (I-107) | 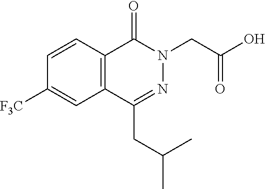 [SMILES: CC(C)Cc1nn(CC(=O)O)c(=O)c2ccc(C(F)(F)F)cc12] (I-71) |
| 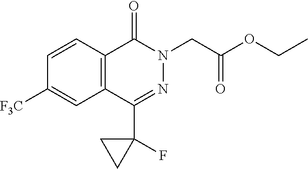 [SMILES: CCOC(=O)Cn1nc(C2(F)CC2)c2cc(C(F)(F)F)ccc2c1=O] (I-108) | 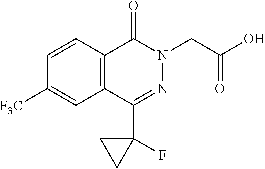 [SMILES: O=C(O)Cn1nc(C2(F)CC2)c2cc(C(F)(F)F)ccc2c1=O] (I-68) |
| 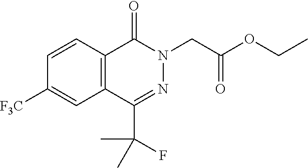 [SMILES: CCOC(=O)Cn1nc(C(C)(C)F)c2cc(C(F)(F)F)ccc2c1=O] (I-112) | 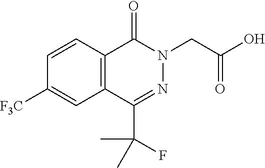 [SMILES: CC(C)(F)c1nn(CC(=O)O)c(=O)c2ccc(C(F)(F)F)cc12] (I-70) |
| 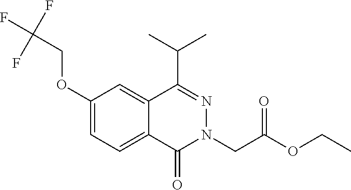 [SMILES: CCOC(=O)Cn1nc(C(C)C)c2cc(OCC(F)(F)F)ccc2c1=O] I-1080 | 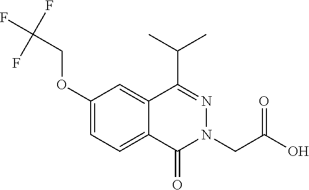 [SMILES: CC(C)c1nn(CC(=O)O)c(=O)c2ccc(OCC(F)(F)F)cc12] I-1116 |
| 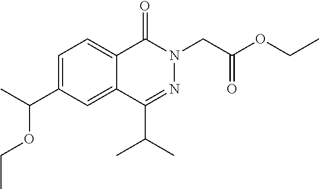 [SMILES: CCOC(=O)Cn1nc(C(C)C)c2cc(C(C)OCC)ccc2c1=O] I-1077 | 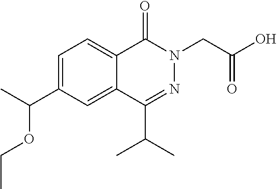 [SMILES: CCOC(C)c1ccc2c(=O)n(CC(=O)O)nc(C(C)C)c2c1] I-1117 |
| 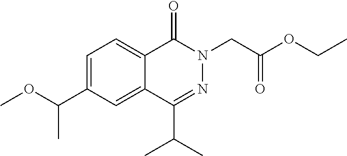 [SMILES: CCOC(=O)Cn1nc(C(C)C)c2cc(C(C)OC)ccc2c1=O] I-1078 | 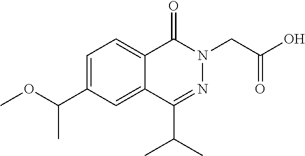 [SMILES: COC(C)c1ccc2c(=O)n(CC(=O)O)nc(C(C)C)c2c1] I-1118 |
| 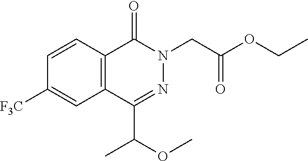 [SMILES: CCOC(=O)Cn1nc(C(C)OC)c2cc(C(F)(F)F)ccc2c1=O] (I-122) | 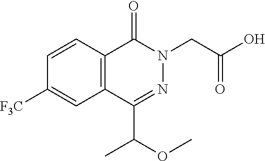 [SMILES: COC(C)c1nn(CC(=O)O)c(=O)c2ccc(C(F)(F)F)cc12] (I-66) |
| 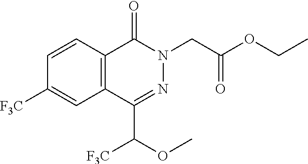 [SMILES: CCOC(=O)Cn1nc(C(OC)C(F)(F)F)c2cc(C(F)(F)F)ccc2c1=O] (I-123) | 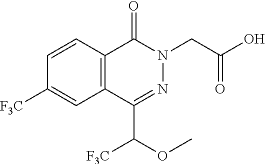 [SMILES: COC(c1nn(CC(=O)O)c(=O)c2ccc(C(F)(F)F)cc12)C(F)(F)F] (I-69) |
| I-1094 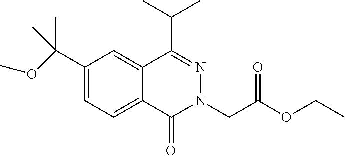 [SMILES: CCOC(=O)Cn1nc(C(C)C)c2cc(C(C)(C)OC)ccc2c1=O] | 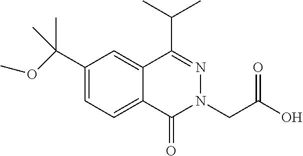 [SMILES: COC(C)(C)c1ccc2c(=O)n(CC(=O)O)nc(C(C)C)c2c1] I-1119 |
| 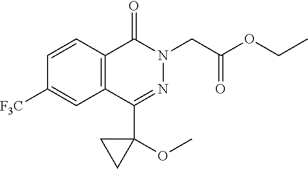 [SMILES: CCOC(=O)Cn1nc(C2(OC)CC2)c2cc(C(F)(F)F)ccc2c1=O] (I-127) | 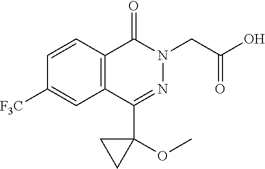 [SMILES: COC1(c2nn(CC(=O)O)c(=O)c3ccc(C(F)(F)F)cc23)CC1] (I-72) |
| 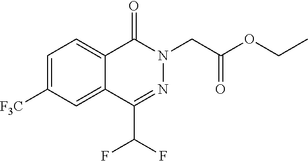 [SMILES: CCOC(=O)Cn1nc(C(F)F)c2cc(C(F)(F)F)ccc2c1=O] I-120 | 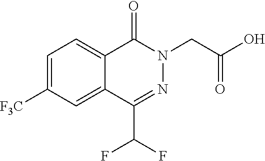 [SMILES: O=C(O)Cn1nc(C(F)F)c2cc(C(F)(F)F)ccc2c1=O] I-60 |
| 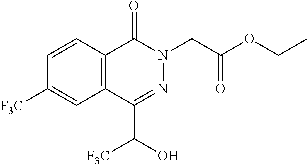 [SMILES: CCOC(=O)Cn1nc(C(O)C(F)(F)F)c2cc(C(F)(F)F)ccc2c1=O] (I-124) | 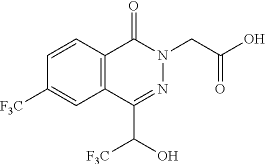 [SMILES: O=C(O)Cn1nc(C(O)C(F)(F)F)c2cc(C(F)(F)F)ccc2c1=O] (I-63) |
| 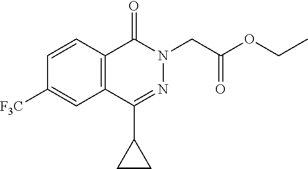 [SMILES: CCOC(=O)Cn1nc(C2CC2)c2cc(C(F)(F)F)ccc2c1=O] (I-130) | 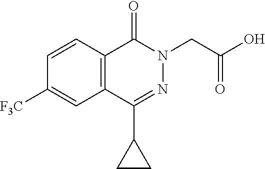 [SMILES: O=C(O)Cn1nc(C2CC2)c2cc(C(F)(F)F)ccc2c1=O] (I-59) |
| 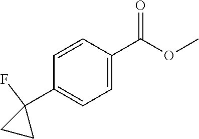 I-1010 | 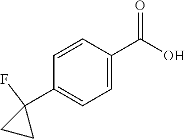 [SMILES: O=C(O)c1ccc(C2(F)CC2)cc1] I-1019 |
| 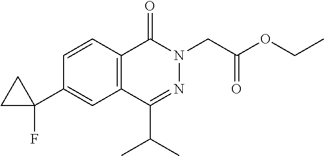 [SMILES: CCOC(=O)Cn1nc(C(C)C)c2cc(C3(F)CC3)ccc2c1=O] I-1090 | 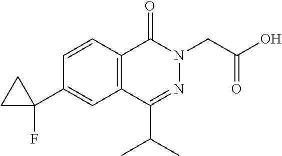 [SMILES: CC(C)c1nn(CC(=O)O)c(=O)c2ccc(C3(F)CC3)cc12] I-1120 |
| 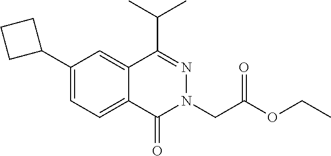 [SMILES: CCOC(=O)Cn1nc(C(C)C)c2cc(C3CCC3)ccc2c1=O] I-1097 | 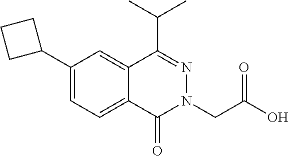 [SMILES: CC(C)c1nn(CC(=O)O)c(=O)c2ccc(C3CCC3)cc12] I-1121 |

### Synthesis of 2-(4-acetyl-1-oxo-6-(trifluoromethyl)phthalazin-2(1H)-yl)acetic acid I-61

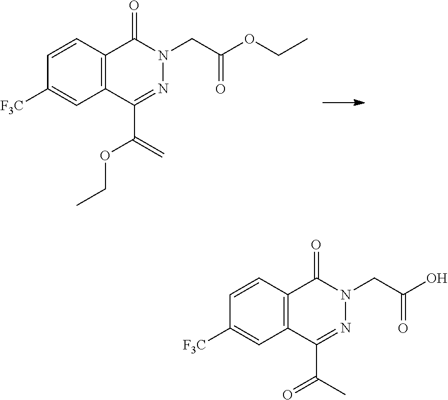 [SMILES: C=C(OCC)c1nn(CC(=O)OCC)c(=O)c2ccc(C(F)(F)F)cc12 ; CC(=O)c1nn(CC(=O)O)c(=O)c2ccc(C(F)(F)F)cc12]

A 1M aqueous solution of NaOH [1310-73-2] (4.3 mL, 4.3 mmol) was added dropwise to a stirred solution of ethyl 2-(4-(1-ethoxyvinyl)-1-oxo-6-(trifluoromethyl)phthalazin-2(1H)-yl)acetate I-80 (797 mg, 2.15 mmol) in MeOH (14.2 mL) and the mixture was stirred at rt for 16 hours. The mixture was acidified with an aqueous HCl solution [7647-01-0] to pH 3-4. It was then extracted with EtOAc and the organic extracts dried over MgSO₄, filtered and concentrated in vacuo. The crude solid was dissolved in 1,4-dioxane (20 mL) and a 6M aqueous solution of HCl [7647-01-0] (1.79 mL, 10.8 mmol) was added dropwise at 10° C. After addition, the mixture was stirred at rt for 1 hour. It was extracted with EtOAc and the organic extracts dried over MgSO₄, filtered and concentrated in vacuo to give I-61 (652 mg, 96%) as a brown solid.

| Intermediate | Product |
|---|---|
| 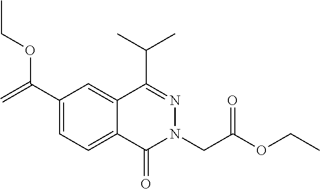 [SMILES: C=C(OCC)c1ccc2c(=O)n(CC(=O)OCC)nc(C(C)C)c2c1] I-1075 | 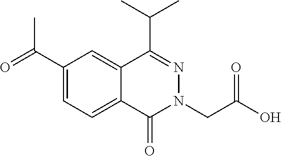 [SMILES: CC(=O)c1ccc2c(=O)n(CC(=O)O)nc(C(C)C)c2c1] I-1051 |

### Synthesis of N-(2,3-dihydro-1H-inden-4-yl)pivalamide I-1122

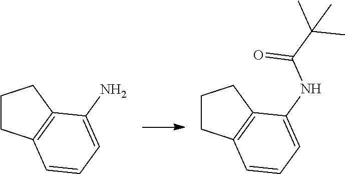 [SMILES: Nc1cccc2c1CCC2 ; CC(C)(C)C(=O)Nc1cccc2c1CCC2]

Pivaloyl chloride [3282-30-2] (1.85 mL, 0.98 g/mL, 15.02 mmol) was added dropwise to a stirred solution of 2,3-dihydro-1H-inden-4-amine [32202-61-2] (2 g, 15.02 mmol) in DCM (25 mL) and triethylamine [121-44-8] (3.14 mL, 0.73 g/mL, 22.52 mmol) at 0° C. Then the reaction was stirred at rt for 1 h. Water was added to the mixture and it was extracted with DCM (×3). The organic layer was separated, dried over MgSO4 anh., filtered and concentrated in vacuo. The product was purified by flash column chromatography (silica; EtOAc in heptane 0/100 to 80/0) to yield N-(2,3-dihydro-1H-inden-4-yl)pivalamide I-1122 (3 g, 91%) as a white solid.

### Synthesis of N-(5-bromo-2,3-dihydro-1H-inden-4-yl)pivalamide I-1123

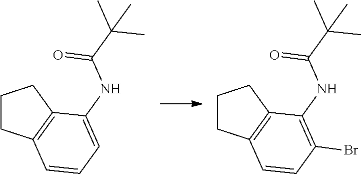 [SMILES: CC(C)(C)C(=O)Nc1cccc2c1CCC2 ; CC(C)(C)C(=O)Nc1c(Br)ccc2c1CCC2]

4-Methylbenzenesulfonic acid [104-15-4] (1.19 g, 6.9 mmol), palladium(II) acetate [3375-31-3] (155 mg, 0.69 mmol) and NBS [128-08-5] (2.21 g, 12.42 mmol) were added to a stirred solution of N-(2,3-dihydro-1H-inden-4-yl)pivalamide I-1122 (3 g, 13.81 mmol) in toluene (23 mL). The mixture was stirred at rt for 16 h. Water was added and the mixture was extracted with DCM (×3). The organic layer was separated, dried over anh. MgSO4, filtered and concentrated in vacuo. The product was purified by flash column chromatography (silica; EtOAc in heptane 0/100 to 80/0). The desired fractions were collected and concentrated in vacuo to yield N-(5-bromo-2,3-dihydro-1H-inden-4-yl)pivalamide I-1123 (4.7 g, 98%) as a white solid.

### Synthesis of 5-bromo-2,3-dihydro-1H-inden-4-amine I-1124

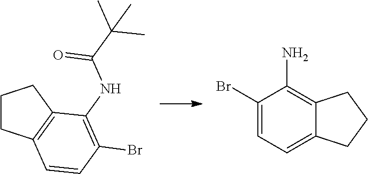 [SMILES: CC(C)(C)C(=O)Nc1c(Br)ccc2c1CCC2 ; Nc1c(Br)ccc2c1CCC2]

N-(5-bromo-2,3-dihydro-1H-inden-4-yl)pivalamide I-1123 (3.47 g, 9.37 mmol) was dissolved in ethanol (22 mL) and stirred at rt. Then sulfuric acid [7664-93-9] (22.05 mL, 390.81 mmol) was slowly added to water (22 mL) and this mixture was added to the reaction mixture. The mixture was stirred at 100° C. over the weekend. The mixture was cooled to rt and then basified with 2M aq. NaOH. The crude mixture was extracted with dichloromethane and then the organic layer was dried over anh. MgSO4, filtered and concentrated in vacuo to yield 5-bromo-2,3-dihydro-1H-inden-4-amine I-1124 (1895 mg, 94%) as a brown oil.

### Synthesis of 5-(2-methoxypyridin-4-yl)-2,3-dihydro-1H-inden-4-amine I-1125

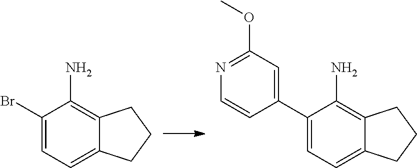 [SMILES: Nc1c(Br)ccc2c1CCC2 ; COc1cc(-c2ccc3c(c2N)CCC3)ccn1]

5-bromo-2,3-dihydro-1H-inden-4-amine I-1124 (1895 mg, 7.15 mmol) was dissolved in dioxane (29 mL) and then potassium carbonate [584-08-7] (2.17 g, 15.73 mmol) in water (5.7 mL) and (2-methoxypyridin-4-yl)boronic acid [762262-09-9] (1.31 g, 8.58 mmol) were added. The mixture was degassed with nitrogen for 15 min and then Pd(dppf)Cl2 CH2Cl2 [95464-05-4] (293 mg, 0.36 mmol) was added. The reaction mixture was heated to 80° C. for 3 hours. The mixture was cooled to room temperature and it was extracted with AcOEt and water. The organic phase was separated, dried over anh. MgSO4, filtered and concentrated in vacuo. The crude product was purified by flash column chromatography (silica; EtOAc in heptane 0/100 to 80/20) to yield 5-(2-methoxypyridin-4-yl)-2,3-dihydro-1H-inden-4-amine I-1125 (1650 mg, 95%) as a beige solid.

### Synthesis of 1-isopropyl-3-nitro-1H-pyrazole I-1086

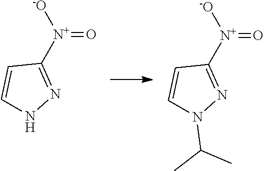

Potassium Carbonate [584-08-7] (59.9 g, 433.34 mmol) and 2-iodopropane [75-30-9](28 mL, 1.7 g/mL, 279.46 mmol) were added to a solution of 3-nitro-1H-pyrazole [26621-44-3] (20 g, 176.87 mmol) in acetonitrile (200 mL). The mixture was stirred at 45° C. for 24 h. The solvent was removed in vacuo (80-90%). Then, a mixture of acetonitrile (31 mL) and MTBE (124 mL) was added and the reaction mixture was stirred for 30 min at rt. The reaction mixture was filtered and washed with acetonitrile and MTBE (2:8). The solvent was removed in vacuo at 45° C. and co-distilled with MTBE at 45° C. The crude product was allowed to stand for 12 h to yield solid crystals. The crystals were dissolved with heptane (124 mL) and stirred for 1.5 h at RT and the mixture was filtered and washed with heptane, and dried 4.5 h to yield 1-isopropyl-3-nitro-1H-pyrazole I-1086 (20.2 g, 73%) as a white solid.

### Synthesis of 2-(4-(1-ethoxyvinyl)-1-oxo-6-(trifluoromethyl)phthalazin-2(1H)-yl)-N-(pyrimidin-4-yl)acetamide I-79

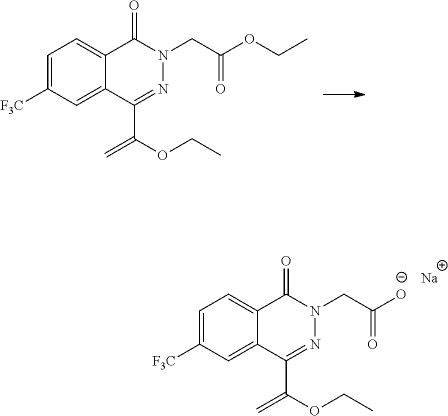 [SMILES: C=C(OCC)c1nn(CC(=O)OCC)c(=O)c2ccc(C(F)(F)F)cc12 ; C=C(OCC)c1nn(CC(=O)[O-])c(=O)c2ccc(C(F)(F)F)cc12 ; [Na+]]

A 1M aqueous solution of NaOH [1310-73-2] (2.33 mL, 2.33 mmol) was added to a solution of ethyl 2-(4-(1-ethoxyvinyl)-1-oxo-6-(trifluoromethyl)phthalazin-2(1H)-yl)acetate (I-80) in MeOH (7.7 mL) and the reaction mixture was stirred at rt for 16 hours. The volatiles were evaporated in vacuo to yield crude sodium 2-(4-(1-ethoxyvinyl)-1-oxo-6-(trifluoromethyl)phthalazin-2(1H)-yl)acetate (I-79) as a bronish solid that was used without further purification.

### Synthesis of 2-(4-(1-ethoxyvinyl)-1-oxo-6-(trifluoromethyl)phthalazin-2(1H)-yl)-N-(pyrimidin-4-yl)acetamide I-78

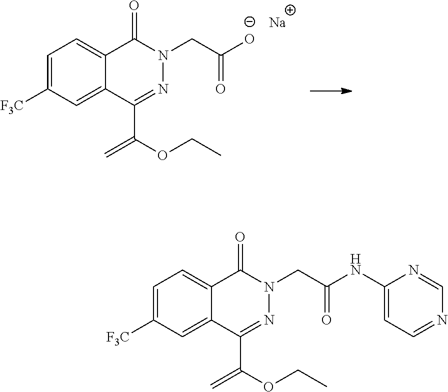 [SMILES: C=C(OCC)c1nn(CC(=O)[O-])c(=O)c2ccc(C(F)(F)F)cc12 ; [Na+] ; C=C(OCC)c1nn(CC(=O)Nc2ccncn2)c(=O)c2ccc(C(F)(F)F)cc12]

Triethylamine [121-44-8] (486 μL, 3.47 mmol) was added to a stirred solution of sodium 2-(4-(1-ethoxyvinyl)-1-oxo-6-(trifluoromethyl)phthalazin-2(1H)-yl)acetate (I-79) (474 mg, 1.16 mmol) and 4-aminopyrimidine [591-54-8] (135 mg, 1.39 mmol) in dry DMF (12.6 mL) at rt under nitrogen. The mixture was stirred at rt for 5 min, then a solution of T3P [68957-94-8] (50% wt in EtOAc, 1.03 mL, 1.74 mmol) was added and the mixture was stirred at rt for 18 h. It was diluted with a saturated aqueous solution of NaHCO₃ and extracted with EtOAc. The organic layer was separated, dried (MgSO4), filtered and the solvents evaporated in vacuo. The crude product was purified by flash column chromatography (DCM/MeOH 0 to 1%) to I-78 (56 mg, 11%) as a yellow solid.

### Synthesis of 2-(4-acetyl-1-oxo-6-(trifluoromethyl)phthalazin-2(1H)-yl)-N-(pyrimidin-4-yl)acetamide I-76

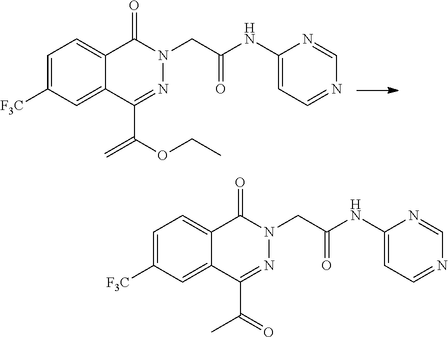

6M aqueous HCl [7647-01-0] (107 μL, 0.64 mmol) was added dropwise to a stirred solution of 2-(4-(1-ethoxyvinyl)-1-oxo-6-(trifluoromethyl)phthalazin-2(1H)-yl)-N-(pyrimidin-4-yl)acetamide (I-78) (54 mg, 0.13 mmol) in 1,4-dioxane (2.9 mL) at 0° C. The mixture was stirred at rt for 1 hour. It was diluted with a saturated aqueous NaHCO₃ solution and extracted with EtOAc. The organic layer was separated, dried (MgSO₄), filtered and the solvents evaporated in vacuo to yield I-76 (42 mg, 82%) as a white solid that was used without further purification.

| Intermediate | Product |
|---|---|
| 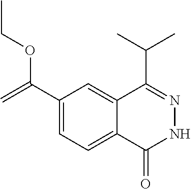 [SMILES: C=C(OCC)c1ccc2c(=O)[nH]nc(C(C)C)c2c1] I-1076 | 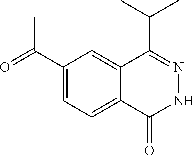 [SMILES: CC(=O)c1ccc2c(=O)[nH]nc(C(C)C)c2c1] I-1053 |
| 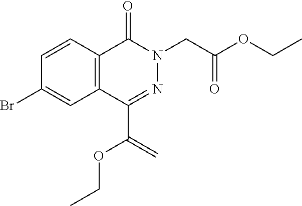 [SMILES: C=C(OCC)c1nn(CC(=O)OCC)c(=O)c2ccc(Br)cc12] I-1074 | 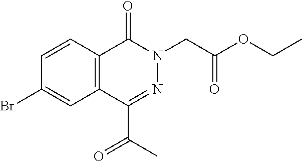 [SMILES: CCOC(=O)Cn1nc(C(C)=O)c2cc(Br)ccc2c1=O] I-1126 |
| 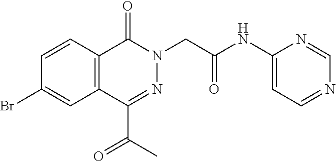 [SMILES: CC(=O)c1nn(CC(=O)Nc2ccncn2)c(=O)c2ccc(Br)cc12] 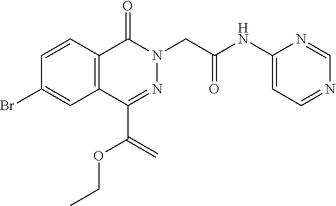 [SMILES: C=C(OCC)c1nn(CC(=O)Nc2ccncn2)c(=O)c2ccc(Br)cc12] I-1128 | 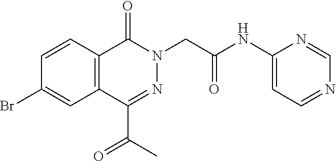 [SMILES: CC(=O)c1nn(CC(=O)Nc2ccncn2)c(=O)c2ccc(Br)cc12] I-1127 |

### Synthesis of 2-(4-isopropyl-1-oxo-6-(trifluoromethyl)phthalazin-2(1H)-yl)acetamide I-77

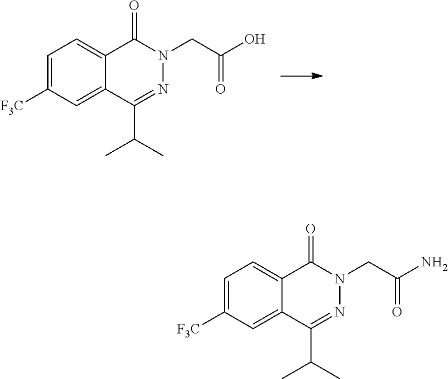 [SMILES: CC(C)c1nn(CC(=O)O)c(=O)c2ccc(C(F)(F)F)cc12 ; CC(C)c1nn(CC(N)=O)c(=O)c2ccc(C(F)(F)F)cc12]

A T3P solution [68957-94-8] (3.61 mL, 50% wt. in EtOAc, 6.05 mmol) was added to a stirred mixture of 2-(4-isopropyl-1-oxo-6-(trifluoromethyl)phthalazin-2(1H)-yl)acetic acid (I-29) (950 mg, 3.02 mmol), 0.5M solution of NH₃ in 1,4-dioxane [7664-41-7] (12 mL, 6.05 mmol) and triethylamine [121-44-8] (1.68 mL, 0.728 g/mL, 12.09 mmol) in 12 mL of anhydrous 1,4-dioxane. After 24 hour at rt, water and ethyl acetate were added. The organic layer was separated, washed with brine, dried (MgSO4), filtered and evaporated under reduced pressure to afford I-77 (948 mg, 87%) as a white solid that was used without further purification.

In general intermediates are labelled with the prefix “I-” and final compounds are indicated as such and may have the prefix “X-”.

### Preparation of Final Compounds

### Example A1

### Synthesis of 2-(6-bromo-4-ethyl-1-oxo-phthalazin-2-yl)-N-pyrimidin-4-yl-acetamide (Final Compound 2)

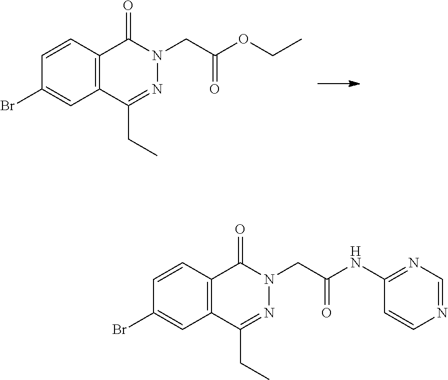 [SMILES: CCOC(=O)Cn1nc(CC)c2cc(Br)ccc2c1=O ; CCc1nn(CC(=O)Nc2ccncn2)c(=O)c2ccc(Br)cc12]

A 2M solution of isopropyl magnesium chloride in THF [1068-55-9] (0.325 mL, 0.65 mmol) was added to a stirred solution of ethyl 2-(6-bromo-4-ethyl-1-oxo-phthalazin-2-yl)acetate (I-28) (0.1 g, 0.29 mmol) and 4-aminopyrimidine [591-54-8] (31 mg, 0.34 mmol) in anhydrous THF (4 mL) at 0° C. under nitrogen. The mixture was stirred at room temperature for 3 h. The mixture was diluted with water at 0° C. and extracted with EtOAc. The organic layer was separated, dried (MgSO4), filtered and the solvents evaporated in vacuo. The crude product was purified by flash column chromatography (silica, MeOH in DCM 0/100 to 10/90). The desired fractions were collected and concentrated in vacuo to yield 2-(6-bromo-4-ethyl-1-oxo-phthalazin-2-yl)-N-pyrimidin-4-yl-acetamide (Final compound 2) (18 mg, 15%) as a white solid.

Additional analogs were accessed using similar reaction conditions, using the appropriate reagent.

| Reagent | Intermediate | Final compound | Number |
|---|---|---|---|
| 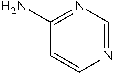 [591-54-8] | 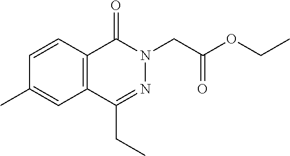 [SMILES: CCOC(=O)Cn1nc(CC)c2cc(C)ccc2c1=O] (I-30) | 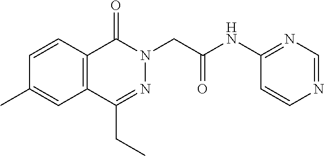 [SMILES: CCc1nn(CC(=O)Nc2ccncn2)c(=O)c2ccc(C)cc12] | 4 |

### Example A2

### Synthesis of N-([1,2,4]triazolo[4,3-b]pyridazin-6-yl)-2-(4-isopropyl-1-oxo-6-(trifluoromethyl)phthalazin-2(1H)-yl)acetamide (Final Compound X2)

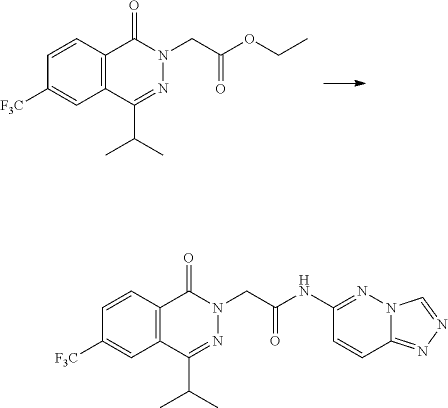 [SMILES: CCOC(=O)Cn1nc(C(C)C)c2cc(C(F)(F)F)ccc2c1=O ; CC(C)c1nn(CC(=O)Nc2ccc3nncn3n2)c(=O)c2ccc(C(F)(F)F)cc12]

[1,2,4]Triazolo[4,3-b]pyridazin-6-amine [19195-46-1] (42.6 mg, 0.32 mmol) was placed in a dry MW vial equipped with a magnetic stir bar and the setup placed under nitrogen (3 vacuum/nitrogen cycles). Anhydrous DMF (0.9 mL) was added and the solution cooled to 0° C. After 10 minutes at 0° C., a solution of LiHMDS (1.0 M in THF, 0.56 mL, 0.56 mmol) was added dropwise and the resulting solution stirred at 0° C. for 15 minutes. Then, a solution of ethyl 2-(4-isopropyl-1-oxo-6-(trifluoromethyl)phthalazin-2(1H)-yl)acetate (1-29) (80 mg, 0.23 mmol) in anhydrous THF (0.76 mL) was added dropwise at 0° C. The resulting mixture was allowed to warm from 0° C. to rt over 1 hour while stirring vigorously and stirred at rt for additional 3 hours. The mixture was concentrated in vacuo (down to 60 mbars, at 50° C.). The obtained glassy residue was purified by preparative HPLC (Stationary phase: RP XBridge Prep C18 OBD-10 μm, 50×250 mm, Mobile phase: 0.25% NH₄HCO₃ solution in water, CH₃CN) to give X2 as a colorless solid (51 mg, 51%).

Note: THF and toluene were used as solvent instead of DMF for the synthesis of some examples, however, DMF is the solvent that generally gives better result with poorly soluble amines.

Additional analogs were accessed using similar reaction conditions, using the appropriate reagent.

| Reagent | Intermediate | Final compound | Number |
|---|---|---|---|
| 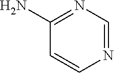 [591-54-8] | 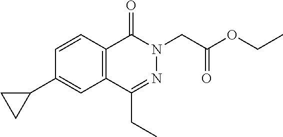 [SMILES: CCOC(=O)Cn1nc(CC)c2cc(C3CC3)ccc2c1=O] (I-32) | 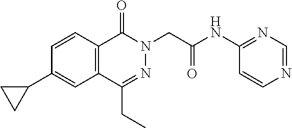 [SMILES: CCc1nn(CC(=O)Nc2ccncn2)c(=O)c2ccc(C3CC3)cc12] | 5 |
| 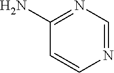 [591-54-8] | 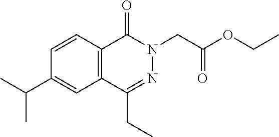 [SMILES: CCOC(=O)Cn1nc(CC)c2cc(C(C)C)ccc2c1=O] (I-35) | 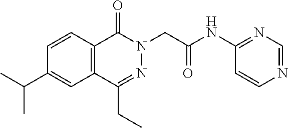 [SMILES: CCc1nn(CC(=O)Nc2ccncn2)c(=O)c2ccc(C(C)C)cc12] | 6 |
| 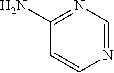 [591-54-8] | 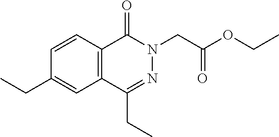 [SMILES: CCOC(=O)Cn1nc(CC)c2cc(CC)ccc2c1=O] (I-36) | 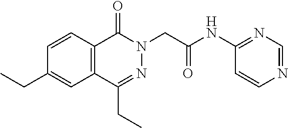 [SMILES: CCc1ccc2c(=O)n(CC(=O)Nc3ccncn3)nc(CC)c2c1] | 7 |
| 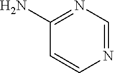 [591-54-8] | 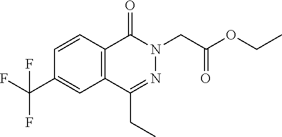 [SMILES: CCOC(=O)Cn1nc(CC)c2cc(C(F)(F)F)ccc2c1=O] (I-25) | 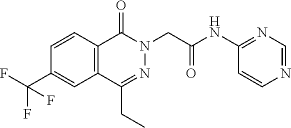 [SMILES: CCc1nn(CC(=O)Nc2ccncn2)c(=O)c2ccc(C(F)(F)F)cc12] | 9 |
| 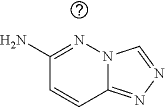 [19195-46-1] | 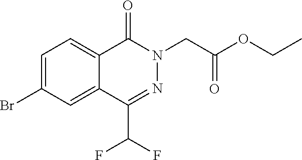 [SMILES: CCOC(=O)Cn1nc(C(F)F)c2cc(Br)ccc2c1=O] I-1091 | 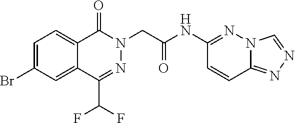 [SMILES: O=C(Cn1nc(C(F)F)c2cc(Br)ccc2c1=O)Nc1ccc2nncn2n1] | X-1000 |
| 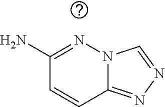 [19195-46-1] | 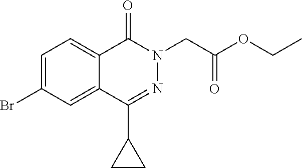 [SMILES: CCOC(=O)Cn1nc(C2CC2)c2cc(Br)ccc2c1=O] I-1057 | 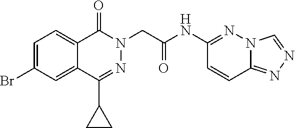 [SMILES: O=C(Cn1nc(C2CC2)c2cc(Br)ccc2c1=O)Nc1ccc2nncn2n1] | X-1001 |
| 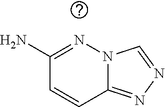 [19195-46-1] | 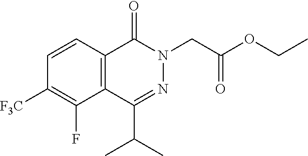 [SMILES: CCOC(=O)Cn1nc(C(C)C)c2c(F)c(C(F)(F)F)ccc2c1=O] (I-88) | 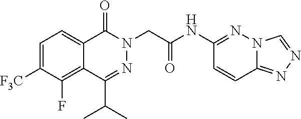 [SMILES: CC(C)c1nn(CC(=O)Nc2ccc3nncn3n2)c(=O)c2ccc(C(F)(F)F)c(F)c12] | X3 |
| 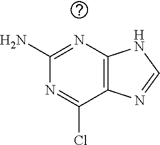 [SMILES: Nc1nc(Cl)c2nc[nH]c2n1] [19195-46-1] | 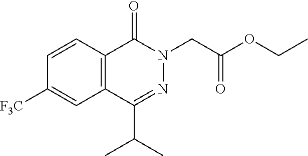 [SMILES: CCOC(=O)Cn1nc(C(C)C)c2cc(C(F)(F)F)ccc2c1=O] (I-29) | 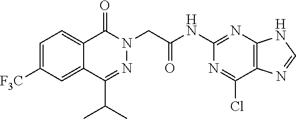 [SMILES: CC(C)c1nn(CC(=O)Nc2nc(Cl)c3nc[nH]c3n2)c(=O)c2ccc(C(F)(F)F)cc12] | X11 |
| 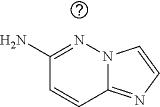 [6653-96-9] | 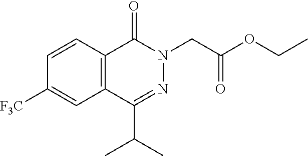 [SMILES: CCOC(=O)Cn1nc(C(C)C)c2cc(C(F)(F)F)ccc2c1=O] (I-29) | 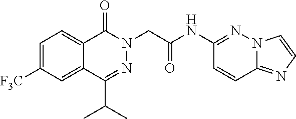 [SMILES: CC(C)c1nn(CC(=O)Nc2ccc3nccn3n2)c(=O)c2ccc(C(F)(F)F)cc12] | X16 |
| 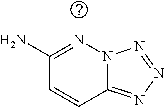 [19195-43-8] | 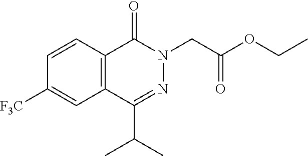 [SMILES: CCOC(=O)Cn1nc(C(C)C)c2cc(C(F)(F)F)ccc2c1=O] (I-29) | 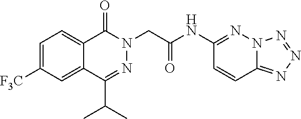 [SMILES: CC(C)c1nn(CC(=O)Nc2ccc3nnnn3n2)c(=O)c2ccc(C(F)(F)F)cc12] | X18 |
| 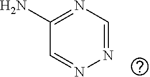 [822-69-5] | 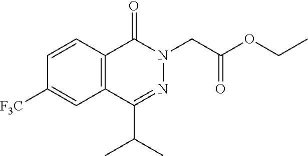 [SMILES: CCOC(=O)Cn1nc(C(C)C)c2cc(C(F)(F)F)ccc2c1=O] (I-29) | 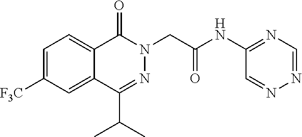 [SMILES: CC(C)c1nn(CC(=O)Nc2cnncn2)c(=O)c2ccc(C(F)(F)F)cc12] | X21 |
| 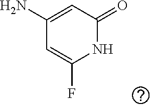 [105252-99-1] | 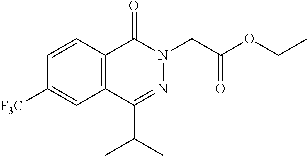 [SMILES: CCOC(=O)Cn1nc(C(C)C)c2cc(C(F)(F)F)ccc2c1=O] (I-29) | 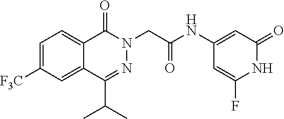 [SMILES: CC(C)c1nn(CC(=O)Nc2cc(F)[nH]c(=O)c2)c(=O)c2ccc(C(F)(F)F)cc12] | X22 |
| 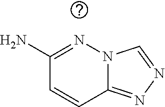 [19195-46-1] | 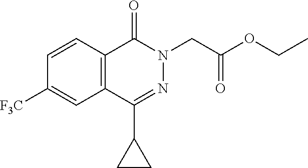 [SMILES: CCOC(=O)Cn1nc(C2CC2)c2cc(C(F)(F)F)ccc2c1=O] (I-130) | 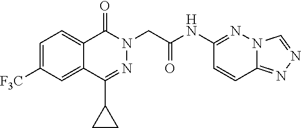 [SMILES: O=C(Cn1nc(C2CC2)c2cc(C(F)(F)F)ccc2c1=O)Nc1ccc2nncn2n1] | X34 |
| 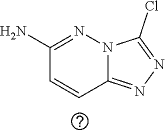 [SMILES: Nc1ccc2nnc(Cl)n2n1] (I-199) | 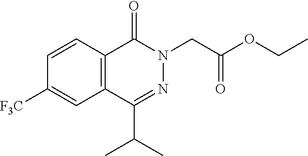 [SMILES: CCOC(=O)Cn1nc(C(C)C)c2cc(C(F)(F)F)ccc2c1=O] (I-29) | 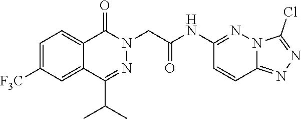 [SMILES: CC(C)c1nn(CC(=O)Nc2ccc3nnc(Cl)n3n2)c(=O)c2ccc(C(F)(F)F)cc12] | X63 |
| 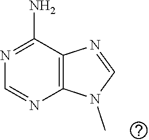 [700-00-5] | 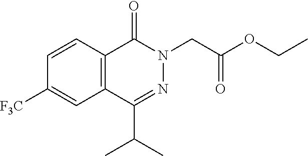 [SMILES: CCOC(=O)Cn1nc(C(C)C)c2cc(C(F)(F)F)ccc2c1=O] (I-29) | 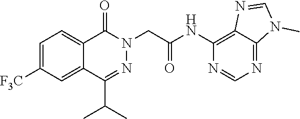 [SMILES: CC(C)c1nn(CC(=O)Nc2ncnc3c2ncn3C)c(=O)c2ccc(C(F)(F)F)cc12] | X69 |
| 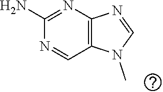 (I-197) | 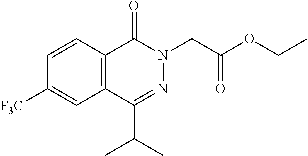 [SMILES: CCOC(=O)Cn1nc(C(C)C)c2cc(C(F)(F)F)ccc2c1=O] (I-29) | 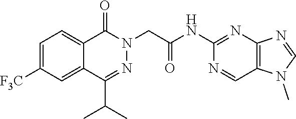 [SMILES: CC(C)c1nn(CC(=O)Nc2ncc3c(ncn3C)n2)c(=O)c2ccc(C(F)(F)F)cc12] | X80 |
| 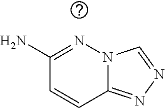 [19195-46-1] | 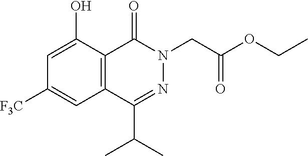 [SMILES: CCOC(=O)Cn1nc(C(C)C)c2cc(C(F)(F)F)cc(O)c2c1=O] (I-74) | 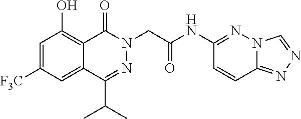 [SMILES: CC(C)c1nn(CC(=O)Nc2ccc3nncn3n2)c(=O)c2c(O)cc(C(F)(F)F)cc12] | X82 |
| 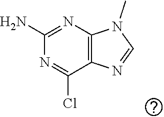 [3035-73-2] | 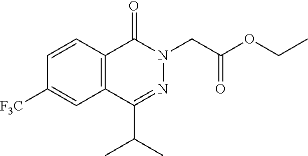 [SMILES: CCOC(=O)Cn1nc(C(C)C)c2cc(C(F)(F)F)ccc2c1=O] (I-29) | 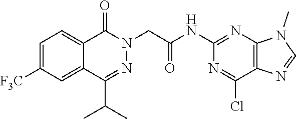 [SMILES: CC(C)c1nn(CC(=O)Nc2nc(Cl)c3ncn(C)c3n2)c(=O)c2ccc(C(F)(F)F)cc12] | X96 |
| 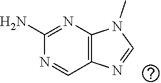 (I-196) | 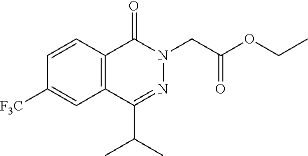 [SMILES: CCOC(=O)Cn1nc(C(C)C)c2cc(C(F)(F)F)ccc2c1=O] (I-29) | 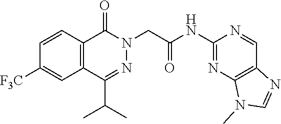 [SMILES: CC(C)c1nn(CC(=O)Nc2ncc3ncn(C)c3n2)c(=O)c2ccc(C(F)(F)F)cc12] | X181 |
| 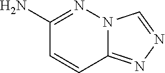 [19195-46-1] | 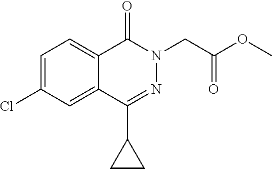 [SMILES: COC(=O)Cn1nc(C2CC2)c2cc(Cl)ccc2c1=O] | 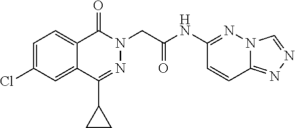 [SMILES: O=C(Cn1nc(C2CC2)c2cc(Cl)ccc2c1=O)Nc1ccc2nncn2n1] | X-1002 |
| 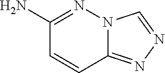 [19195-46-1] | 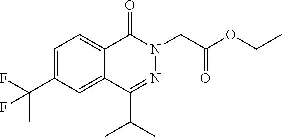 [SMILES: CCOC(=O)Cn1nc(C(C)C)c2cc(C(C)(F)F)ccc2c1=O] I-1098 | 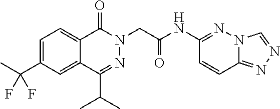 [SMILES: CC(C)c1nn(CC(=O)Nc2ccc3nncn3n2)c(=O)c2ccc(C(C)(F)F)cc12] | X-1003 |
| 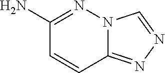 [19195-46-1] | 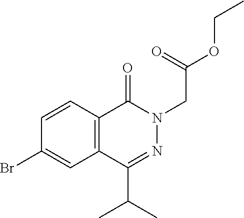 [SMILES: CCOC(=O)Cn1nc(C(C)C)c2cc(Br)ccc2c1=O] | 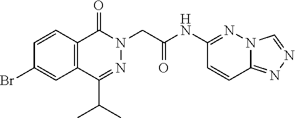 [SMILES: CC(C)c1nn(CC(=O)Nc2ccc3nncn3n2)c(=O)c2ccc(Br)cc12] | X-1004 |
| 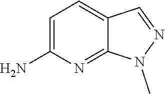 | 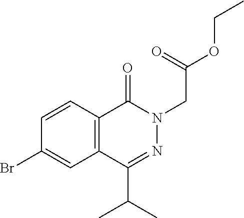 [SMILES: CCOC(=O)Cn1nc(C(C)C)c2cc(Br)ccc2c1=O] | 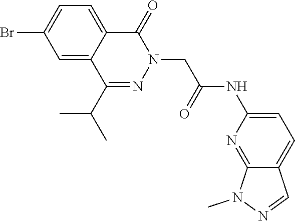 [SMILES: CC(C)c1nn(CC(=O)Nc2ccc3cnn(C)c3n2)c(=O)c2ccc(Br)cc12] | X-1005 |
| 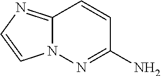 [6653-96-9] | 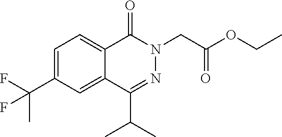 [SMILES: CCOC(=O)Cn1nc(C(C)C)c2cc(C(C)(F)F)ccc2c1=O] I-1098 | 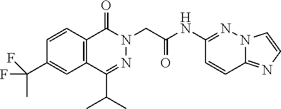 [SMILES: CC(C)c1nn(CC(=O)Nc2ccc3nccn3n2)c(=O)c2ccc(C(C)(F)F)cc12] | X-1006 |
| 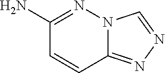 [19195-46-1] | I-1090 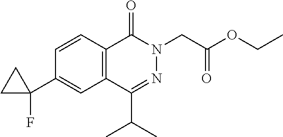 [SMILES: CCOC(=O)Cn1nc(C(C)C)c2cc(C3(F)CC3)ccc2c1=O] | 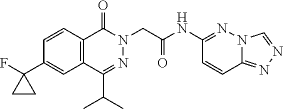 [SMILES: CC(C)c1nn(CC(=O)Nc2ccc3nncn3n2)c(=O)c2ccc(C3(F)CC3)cc12] | X-1007 |
| 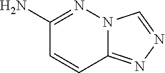 [19195-46-1] | 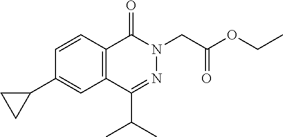 [SMILES: CCOC(=O)Cn1nc(C(C)C)c2cc(C3CC3)ccc2c1=O] | 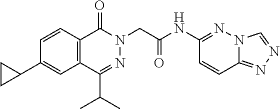 [SMILES: CC(C)c1nn(CC(=O)Nc2ccc3nncn3n2)c(=O)c2ccc(C3CC3)cc12] | X-1008 |
| 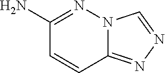 [19195-46-1] | 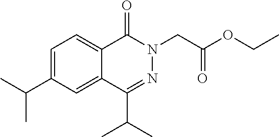 [SMILES: CCOC(=O)Cn1nc(C(C)C)c2cc(C(C)C)ccc2c1=O] | 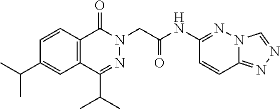 [SMILES: CC(C)c1ccc2c(=O)n(CC(=O)Nc3ccc4nncn4n3)nc(C(C)C)c2c1] | X-1009 |
| 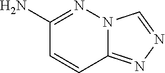 [19195-46-1] | 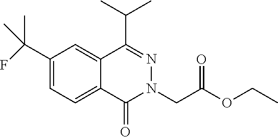 [SMILES: CCOC(=O)Cn1nc(C(C)C)c2cc(C(C)(C)F)ccc2c1=O] I-1096 | 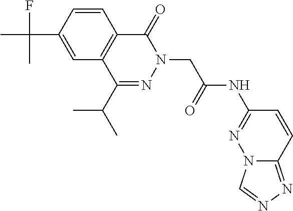 [SMILES: CC(C)c1nn(CC(=O)Nc2ccc3nncn3n2)c(=O)c2ccc(C(C)(C)F)cc12] | X-1010 |
| 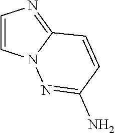 [6653-96-9] | 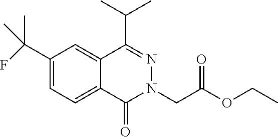 [SMILES: CCOC(=O)Cn1nc(C(C)C)c2cc(C(C)(C)F)ccc2c1=O] I-1096 | 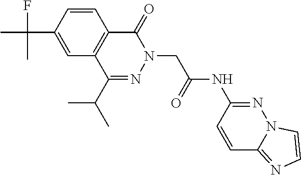 [SMILES: CC(C)c1nn(CC(=O)Nc2ccc3nccn3n2)c(=O)c2ccc(C(C)(C)F)cc12] | X-1011 |
| 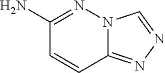 [19195-46-1] | 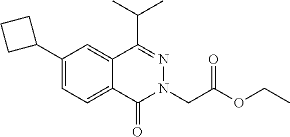 [SMILES: CCOC(=O)Cn1nc(C(C)C)c2cc(C3CCC3)ccc2c1=O] | 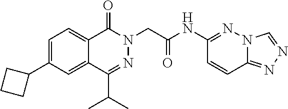 [SMILES: CC(C)c1nn(CC(=O)Nc2ccc3nncn3n2)c(=O)c2ccc(C3CCC3)cc12] | X-1012 |

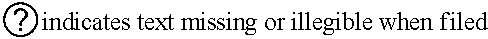

### Example A3

### Synthesis of 2-(4-isopropyl-6-methoxy-1-oxo-phthalazin-2-yl)-N-pyrimidin-4-yl-acetamide (Final Compound 10)

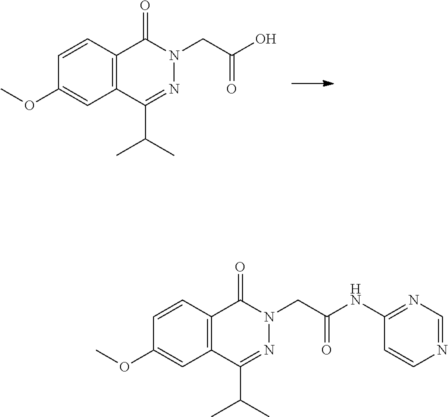 [SMILES: COc1ccc2c(=O)n(CC(=O)O)nc(C(C)C)c2c1 ; COc1ccc2c(=O)n(CC(=O)Nc3ccncn3)nc(C(C)C)c2c1]

4-Aminopyrimidine [591-54-8] (28 mg, 0.29 mmol) was added to a stirred solution of 2-(4-isopropyl-6-methoxy-1-oxo-phthalazin-2-yl)acetic acid (I-39) (56 mg, 0.2 mmol), 1-propanephosphonic anhydride [68957-94-8] (0.3 mL, 0.47 mmol) and triethylamine [121-44-8] (0.1 mL, 0.72 mmol) in anhydrous DCM (3 mL). The mixture was stirred at room temperature for 4 h. The mixture was diluted with saturated aqueous solution of Na₂CO₃ and extracted with DCM. The organic layer was separated, dried (MgSO4), filtered and the solvents evaporated in vacuo. The crude product was purified by flash column chromatography (silica, EtOAc in heptane 0/100 to 100/0). The desired fractions were collected and concentrated in vacuo to yield 2-(4-isopropyl-6-methoxy-1-oxo-phthalazin-2-yl)-N-pyrimidin-4-yl-acetamide (Final compound 10) (45 mg, 63%) as white solid.

Note: DCM and DMF can be used indiscriminately as solvent in this reaction.

Additional analogs were accessed using similar reaction conditions, using the appropriate reagent.

| Reagent | Intermediate | Final compound | Number |
|---|---|---|---|
| 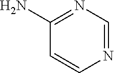 [591-54-8] | 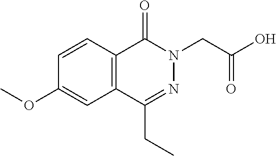 [SMILES: CCc1nn(CC(=O)O)c(=O)c2ccc(OC)cc12] (I-40) | 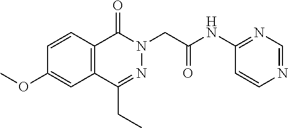 [SMILES: CCc1nn(CC(=O)Nc2ccncn2)c(=O)c2ccc(OC)cc12] | 11 |
| 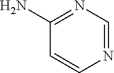 [591-54-8] | 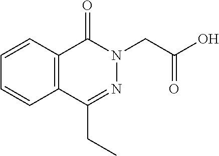 [SMILES: CCc1nn(CC(=O)O)c(=O)c2ccccc12] (I-41) | 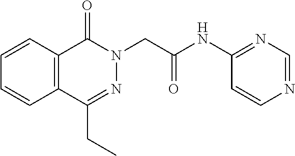 [SMILES: CCc1nn(CC(=O)Nc2ccncn2)c(=O)c2ccccc12] | 12 |
| 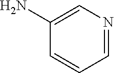 [462-08-8] | 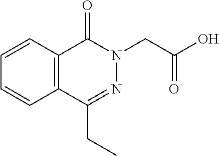 [SMILES: CCc1nn(CC(=O)O)c(=O)c2ccccc12] (I-41) | 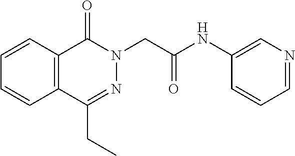 [SMILES: CCc1nn(CC(=O)Nc2cccnc2)c(=O)c2ccccc12] | 13 |
| 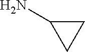 [765-30-0] | 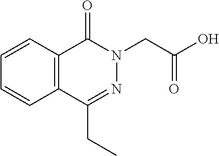 [SMILES: CCc1nn(CC(=O)O)c(=O)c2ccccc12] (I-41) | 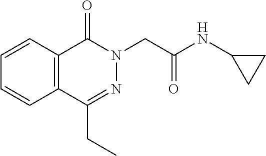 [SMILES: CCc1nn(CC(=O)NC2CC2)c(=O)c2ccccc12] | 14 |
| 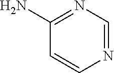 [591-54-8] | 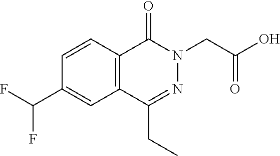 [SMILES: CCc1nn(CC(=O)O)c(=O)c2ccc(C(F)F)cc12] (I-50) | 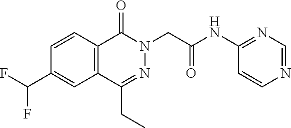 [SMILES: CCc1nn(CC(=O)Nc2ccncn2)c(=O)c2ccc(C(F)F)cc12] | 16 |
| 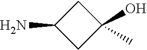 [1363381-58-1] | 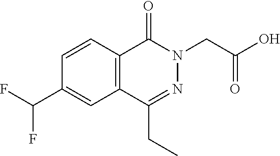 [SMILES: CCc1nn(CC(=O)O)c(=O)c2ccc(C(F)F)cc12] (I-50) | 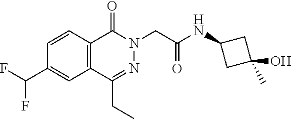 [SMILES: CCc1nn(CC(=O)N[C@H]2C[C@@](C)(O)C2)c(=O)c2ccc(C(F)F)cc12] | 17 |
| 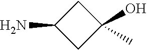 [1363381-58-1] | 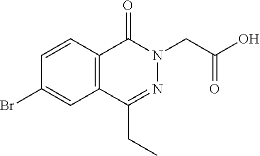 [SMILES: CCc1nn(CC(=O)O)c(=O)c2ccc(Br)cc12] (I-44) | 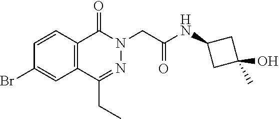 [SMILES: CCc1nn(CC(=O)N[C@H]2C[C@@](C)(O)C2)c(=O)c2ccc(Br)cc12] | 18 |
| 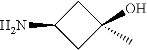 [1363381-58-1] | 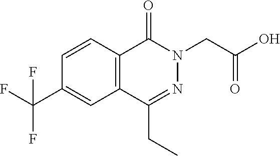 [SMILES: CCc1nn(CC(=O)O)c(=O)c2ccc(C(F)(F)F)cc12] (I-51) | 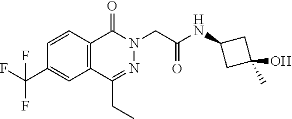 [SMILES: CCc1nn(CC(=O)N[C@H]2C[C@@](C)(O)C2)c(=O)c2ccc(C(F)(F)F)cc12] | 19 |
| 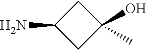 [1363381-58-1] | 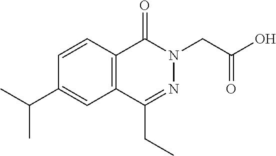 [SMILES: CCc1nn(CC(=O)O)c(=O)c2ccc(C(C)C)cc12] (I-48) | 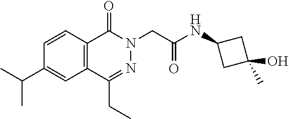 [SMILES: CCc1nn(CC(=O)N[C@H]2C[C@@](C)(O)C2)c(=O)c2ccc(C(C)C)cc12] | 20 |
| 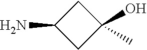 [1363381-58-1] | 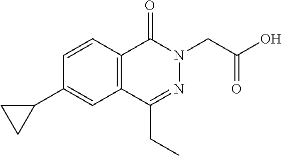 [SMILES: CCc1nn(CC(=O)O)c(=O)c2ccc(C3CC3)cc12] (I-51) | 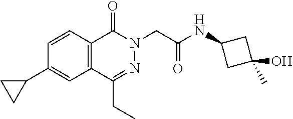 [SMILES: CCc1nn(CC(=O)N[C@H]2C[C@@](C)(O)C2)c(=O)c2ccc(C3CC3)cc12] | 21 |
| 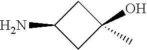 [1363381-58-1] | 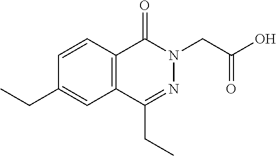 [SMILES: CCc1ccc2c(=O)n(CC(=O)O)nc(CC)c2c1] (I-49) | 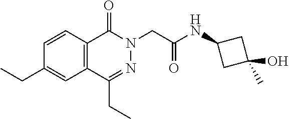 [SMILES: CCc1ccc2c(=O)n(CC(=O)N[C@H]3C[C@@](C)(O)C3)nc(CC)c2c1] | 22 |
| 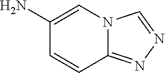 [1087448-58-5] | 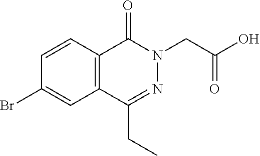 [SMILES: CCc1nn(CC(=O)O)c(=O)c2ccc(Br)cc12] (I-44) | 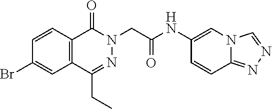 [SMILES: CCc1nn(CC(=O)Nc2ccc3nncn3c2)c(=O)c2ccc(Br)cc12] | 1 |
| 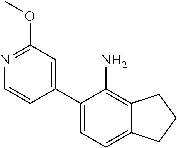 [SMILES: COc1cc(-c2ccc3c(c2N)CCC3)ccn1] I-1125 | 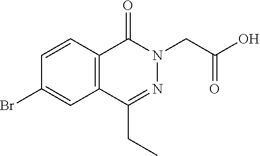 [SMILES: CCc1nn(CC(=O)O)c(=O)c2ccc(Br)cc12] (I-44) | 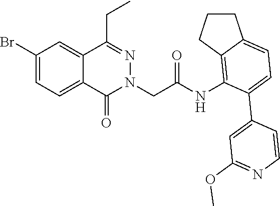 [SMILES: CCc1nn(CC(=O)Nc2c(-c3ccnc(OC)c3)ccc3c2CCC3)c(=O)c2ccc(Br)cc12] | X-1013 |
| 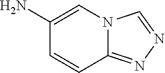 [1087448-58-5] | 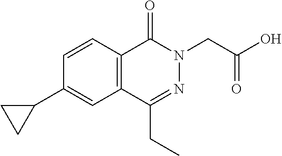 [SMILES: CCc1nn(CC(=O)O)c(=O)c2ccc(C3CC3)cc12] (I-51) | 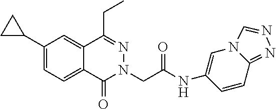 [SMILES: CCc1nn(CC(=O)Nc2ccc3nncn3c2)c(=O)c2ccc(C3CC3)cc12] | 3 |
| 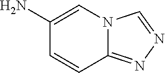 [1087448-58-5] | 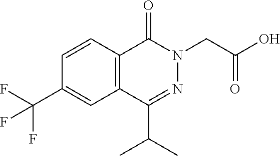 [SMILES: CC(C)c1nn(CC(=O)O)c(=O)c2ccc(C(F)(F)F)cc12] (I-52) | 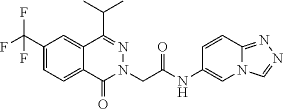 [SMILES: CC(C)c1nn(CC(=O)Nc2ccc3nncn3c2)c(=O)c2ccc(C(F)(F)F)cc12] | 8 |
| 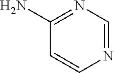 [591-54-8] | 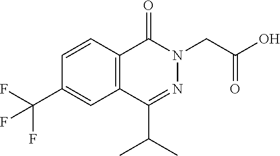 [SMILES: CC(C)c1nn(CC(=O)O)c(=O)c2ccc(C(F)(F)F)cc12] (I-52) | 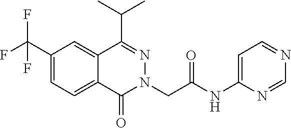 [SMILES: CC(C)c1nn(CC(=O)Nc2ccncn2)c(=O)c2ccc(C(F)(F)F)cc12] | 15 |
| 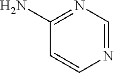 [591-54-8] | 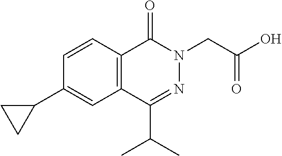 [SMILES: CC(C)c1nn(CC(=O)O)c(=O)c2ccc(C3CC3)cc12] (I-54) | 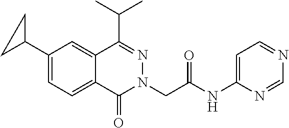 [SMILES: CC(C)c1nn(CC(=O)Nc2ccncn2)c(=O)c2ccc(C3CC3)cc12] | 23 |
| 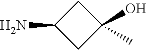 [1363381-58-1] | 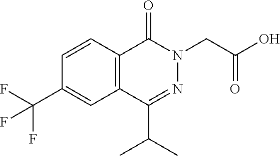 [SMILES: CC(C)c1nn(CC(=O)O)c(=O)c2ccc(C(F)(F)F)cc12] (I-52) | 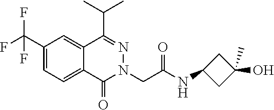 [SMILES: CC(C)c1nn(CC(=O)N[C@H]2C[C@@](C)(O)C2)c(=O)c2ccc(C(F)(F)F)cc12] | 25 |
| 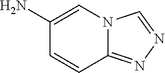 [1087448-58-5] | 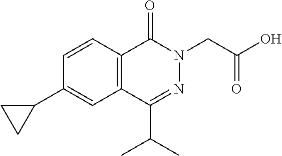 [SMILES: CC(C)c1nn(CC(=O)O)c(=O)c2ccc(C3CC3)cc12] (I-54) | 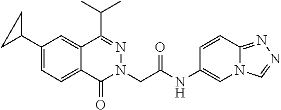 [SMILES: CC(C)c1nn(CC(=O)Nc2ccc3nncn3c2)c(=O)c2ccc(C3CC3)cc12] | 31 |
| 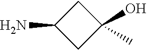 [1363381-58-1] | 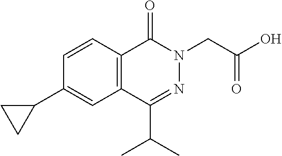 [SMILES: CC(C)c1nn(CC(=O)O)c(=O)c2ccc(C3CC3)cc12] (I-54) | 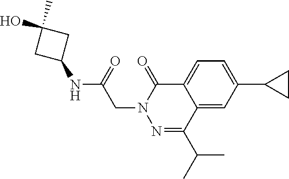 [SMILES: CC(C)c1nn(CC(=O)N[C@H]2C[C@@](C)(O)C2)c(=O)c2ccc(C3CC3)cc12] | 32 |
| 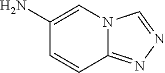 [1087448-58-5] | 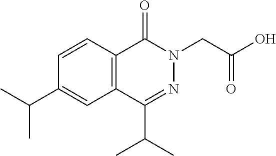 [SMILES: CC(C)c1ccc2c(=O)n(CC(=O)O)nc(C(C)C)c2c1] (I-55) | 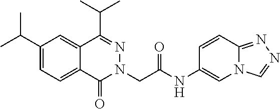 [SMILES: CC(C)c1ccc2c(=O)n(CC(=O)Nc3ccc4nncn4c3)nc(C(C)C)c2c1] | 33 |
| 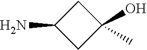 [1363381-58-1] | 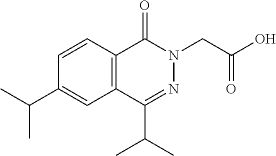 [SMILES: CC(C)c1ccc2c(=O)n(CC(=O)O)nc(C(C)C)c2c1] (I-55) | 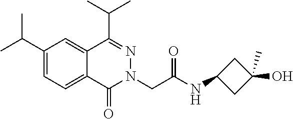 [SMILES: CC(C)c1ccc2c(=O)n(CC(=O)N[C@H]3C[C@@](C)(O)C3)nc(C(C)C)c2c1] | 34 |
| 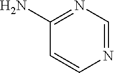 [591-54-8] | 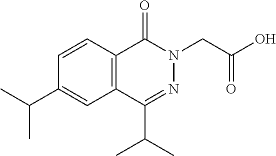 [SMILES: CC(C)c1ccc2c(=O)n(CC(=O)O)nc(C(C)C)c2c1] (I-55) | 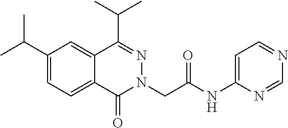 [SMILES: CC(C)c1ccc2c(=O)n(CC(=O)Nc3ccncn3)nc(C(C)C)c2c1] | 35 |
| 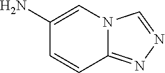 [1087448-58-5] | 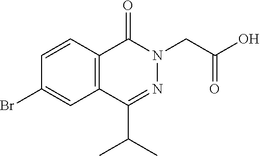 [SMILES: CC(C)c1nn(CC(=O)O)c(=O)c2ccc(Br)cc12] (I-53) | 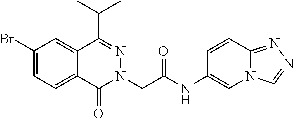 [SMILES: CC(C)c1nn(CC(=O)Nc2ccc3nncn3c2)c(=O)c2ccc(Br)cc12] | 36 |
| 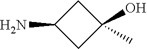 [1363381-58-1] | 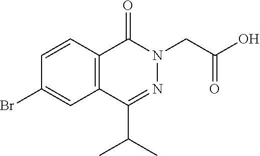 [SMILES: CC(C)c1nn(CC(=O)O)c(=O)c2ccc(Br)cc12] (I-53) | 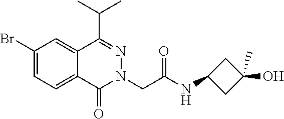 [SMILES: CC(C)c1nn(CC(=O)N[C@H]2C[C@@](C)(O)C2)c(=O)c2ccc(Br)cc12] | 37 |
| 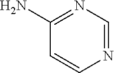 [591-54-8] | 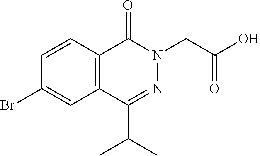 [SMILES: CC(C)c1nn(CC(=O)O)c(=O)c2ccc(Br)cc12] (I-53) | 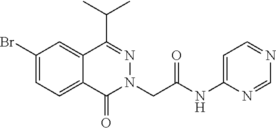 [SMILES: CC(C)c1nn(CC(=O)Nc2ccncn2)c(=O)c2ccc(Br)cc12] | 38 |
| 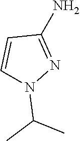 I-1087 | 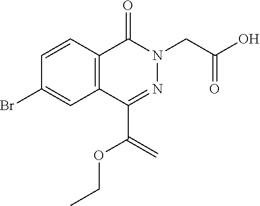 [SMILES: C=C(OCC)c1nn(CC(=O)O)c(=O)c2ccc(Br)cc12] I-1101 | 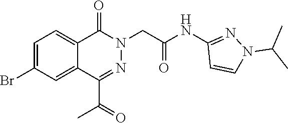 [SMILES: CC(=O)c1nn(CC(=O)Nc2ccn(C(C)C)n2)c(=O)c2ccc(Br)cc12] | X-1014 |
| 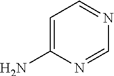 [591-54-8] | 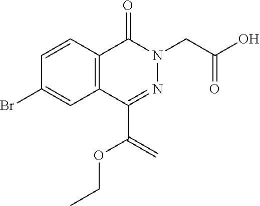 [SMILES: C=C(OCC)c1nn(CC(=O)O)c(=O)c2ccc(Br)cc12] I-1101 | 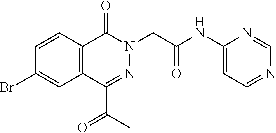 [SMILES: CC(=O)c1nn(CC(=O)Nc2ccncn2)c(=O)c2ccc(Br)cc12] 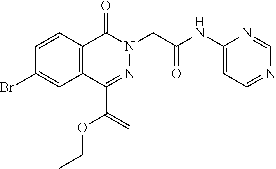 [SMILES: C=C(OCC)c1nn(CC(=O)Nc2ccncn2)c(=O)c2ccc(Br)cc12] | I-1128 |
| 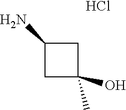 [1523606-23-6] | 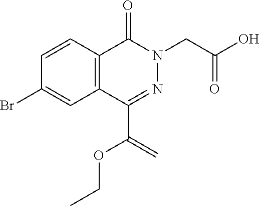 [SMILES: C=C(OCC)c1nn(CC(=O)O)c(=O)c2ccc(Br)cc12] I-1101 | 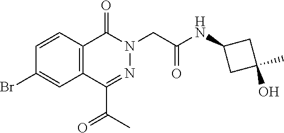 [SMILES: CC(=O)c1nn(CC(=O)N[C@H]2C[C@@](C)(O)C2)c(=O)c2ccc(Br)cc12] | I-1129 |
| 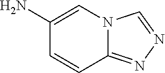 [1087448-58-5] | 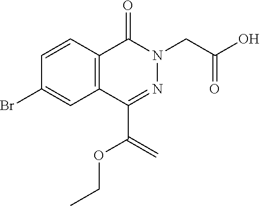 [SMILES: C=C(OCC)c1nn(CC(=O)O)c(=O)c2ccc(Br)cc12] I-1101 | 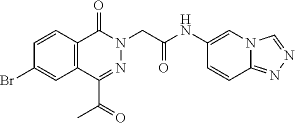 [SMILES: CC(=O)c1nn(CC(=O)Nc2ccc3nncn3c2)c(=O)c2ccc(Br)cc12] | I-1130 |
| 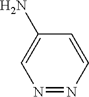 [20744-39-2] | 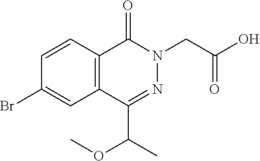 [SMILES: COC(C)c1nn(CC(=O)O)c(=O)c2ccc(Br)cc12] I-1102 | 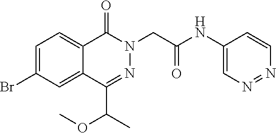 [SMILES: COC(C)c1nn(CC(=O)Nc2ccnnc2)c(=O)c2ccc(Br)cc12] | X-1015 |
| 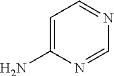 [591-54-8] | 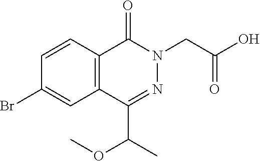 [SMILES: COC(C)c1nn(CC(=O)O)c(=O)c2ccc(Br)cc12] I-1102 | 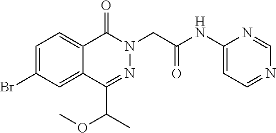 [SMILES: COC(C)c1nn(CC(=O)Nc2ccncn2)c(=O)c2ccc(Br)cc12] | X-1016 |
| 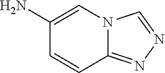 [1087448-58-5] | 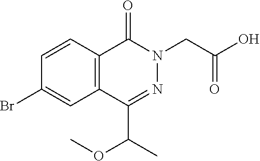 [SMILES: COC(C)c1nn(CC(=O)O)c(=O)c2ccc(Br)cc12] I-1102 | 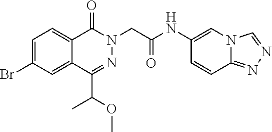 [SMILES: COC(C)c1nn(CC(=O)Nc2ccc3nncn3c2)c(=O)c2ccc(Br)cc12] | X-1017 |
| 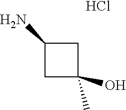 [1523606-23-6] | 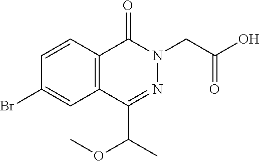 [SMILES: COC(C)c1nn(CC(=O)O)c(=O)c2ccc(Br)cc12] I-1102 | 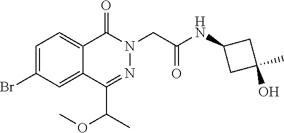 [SMILES: COC(C)c1nn(CC(=O)N[C@H]2C[C@@](C)(O)C2)c(=O)c2ccc(Br)cc12] | X-1018 |
| 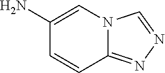 [1087448-58-5] | 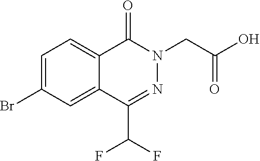 [SMILES: O=C(O)Cn1nc(C(F)F)c2cc(Br)ccc2c1=O] I-1114 | 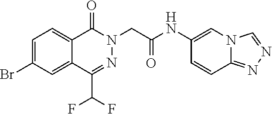 [SMILES: O=C(Cn1nc(C(F)F)c2cc(Br)ccc2c1=O)Nc1ccc2nncn2c1] | X-1019 |
| 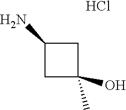 [1523606-23-6] | 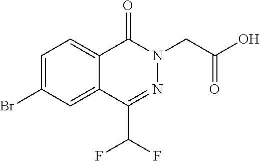 [SMILES: O=C(O)Cn1nc(C(F)F)c2cc(Br)ccc2c1=O] I-1114 | 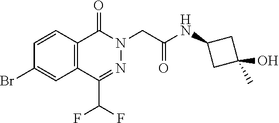 [SMILES: C[C@]1(O)C[C@@H](NC(=O)Cn2nc(C(F)F)c3cc(Br)ccc3c2=O)C1] | X-1020 |
| 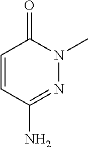 [13506-28-0] | 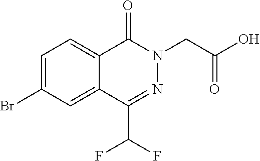 [SMILES: O=C(O)Cn1nc(C(F)F)c2cc(Br)ccc2c1=O] I-1114 | 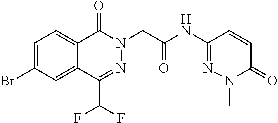 [SMILES: Cn1nc(NC(=O)Cn2nc(C(F)F)c3cc(Br)ccc3c2=O)ccc1=O] | X-1021 |
| 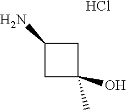 [1523606-23-6] | 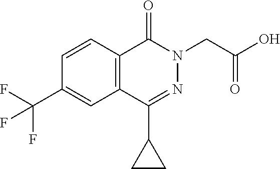 [SMILES: O=C(O)Cn1nc(C2CC2)c2cc(C(F)(F)F)ccc2c1=O] (I-59) | 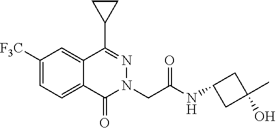 [SMILES: C[C@]1(O)C[C@@H](NC(=O)Cn2nc(C3CC3)c3cc(C(F)(F)F)ccc3c2=O)C1] | X4 |
| 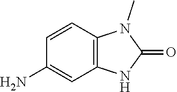 [54732-89-7] | 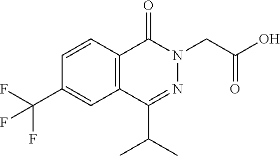 [SMILES: CC(C)c1nn(CC(=O)O)c(=O)c2ccc(C(F)(F)F)cc12] (I-52) | 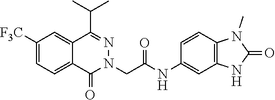 [SMILES: CC(C)c1nn(CC(=O)Nc2ccc3c(c2)[nH]c(=O)n3C)c(=O)c2ccc(C(F)(F)F)cc12] | X5 |
| 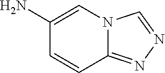 [1087448-58-5] | 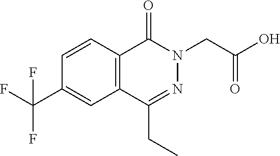 [SMILES: CCc1nn(CC(=O)O)c(=O)c2ccc(C(F)(F)F)cc12] (I-51) | 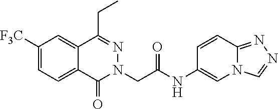 [SMILES: CCc1nn(CC(=O)Nc2ccc3nncn3c2)c(=O)c2ccc(C(F)(F)F)cc12] | X8 |
| 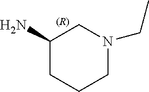 [1020396-26-2] | 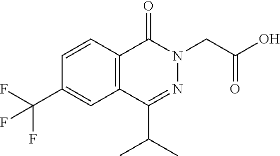 [SMILES: CC(C)c1nn(CC(=O)O)c(=O)c2ccc(C(F)(F)F)cc12] (I-52) | 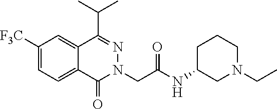 [SMILES: CCN1CCC[C@@H](NC(=O)Cn2nc(C(C)C)c3cc(C(F)(F)F)ccc3c2=O)C1] | X9 |
| 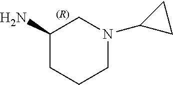 (I-200) | 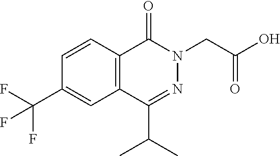 [SMILES: CC(C)c1nn(CC(=O)O)c(=O)c2ccc(C(F)(F)F)cc12] (I-52) | 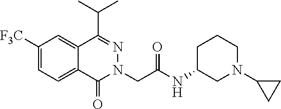 [SMILES: CC(C)c1nn(CC(=O)N[C@@H]2CCCN(C3CC3)C2)c(=O)c2ccc(C(F)(F)F)cc12] | X10 |
| 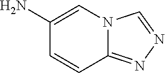 [1087448-58-5] | 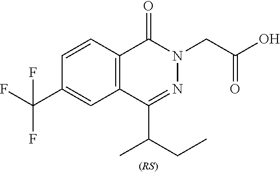 [SMILES: CCC(C)c1nn(CC(=O)O)c(=O)c2ccc(C(F)(F)F)cc12] (I-57) | 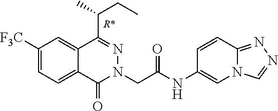 [SMILES: CC[C@@H](C)c1nn(CC(=O)Nc2ccc3nncn3c2)c(=O)c2ccc(C(F)(F)F)cc12] | X12 |
| 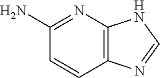 [69825-84-9] | 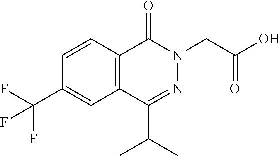 [SMILES: CC(C)c1nn(CC(=O)O)c(=O)c2ccc(C(F)(F)F)cc12] (I-52) | 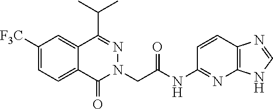 [SMILES: CC(C)c1nn(CC(=O)Nc2ccc3nc[nH]c3n2)c(=O)c2ccc(C(F)(F)F)cc12] | X13 |
| 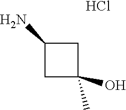 [1523606-23-6] | 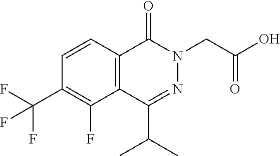 [SMILES: CC(C)c1nn(CC(=O)O)c(=O)c2ccc(C(F)(F)F)c(F)c12] (I-58) | 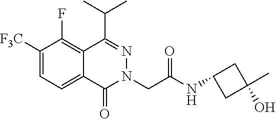 [SMILES: CC(C)c1nn(CC(=O)N[C@H]2C[C@@](C)(O)C2)c(=O)c2ccc(C(F)(F)F)c(F)c12] | X17 |
| 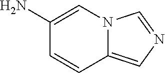 [1508379-00-7] | 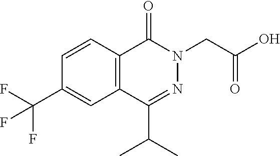 [SMILES: CC(C)c1nn(CC(=O)O)c(=O)c2ccc(C(F)(F)F)cc12] (I-52) | 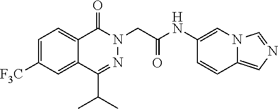 [SMILES: CC(C)c1nn(CC(=O)Nc2ccc3cncn3c2)c(=O)c2ccc(C(F)(F)F)cc12] | X20 |
| 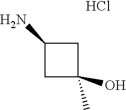 [1523606-23-6] | 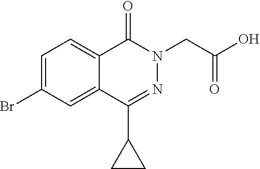 [SMILES: O=C(O)Cn1nc(C2CC2)c2cc(Br)ccc2c1=O] I-1103 | 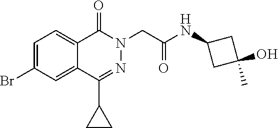 [SMILES: C[C@]1(O)C[C@@H](NC(=O)Cn2nc(C3CC3)c3cc(Br)ccc3c2=O)C1] | X-1022 |
| 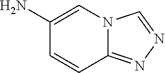 [1082448-58-5] | 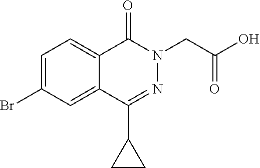 [SMILES: O=C(O)Cn1nc(C2CC2)c2cc(Br)ccc2c1=O] I-1103 | 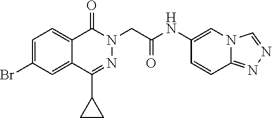 [SMILES: O=C(Cn1nc(C2CC2)c2cc(Br)ccc2c1=O)Nc1ccc2nncn2c1] | X-1023 |
| 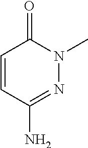 [13506-28-0] | 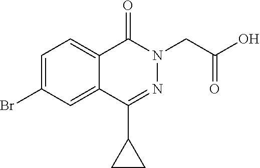 [SMILES: O=C(O)Cn1nc(C2CC2)c2cc(Br)ccc2c1=O] I-1103 | 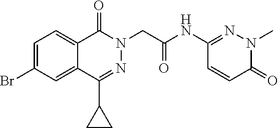 [SMILES: Cn1nc(NC(=O)Cn2nc(C3CC3)c3cc(Br)ccc3c2=O)ccc1=O] | X-1024 |
| 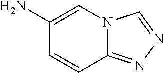 [1087448-58-5] | 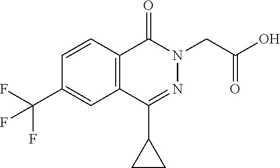 [SMILES: O=C(O)Cn1nc(C2CC2)c2cc(C(F)(F)F)ccc2c1=O] (I-59) | 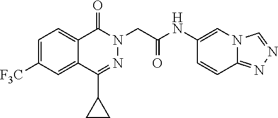 [SMILES: O=C(Cn1nc(C2CC2)c2cc(C(F)(F)F)ccc2c1=O)Nc1ccc2nncn2c1] | X23 |
| 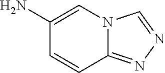 [1087448-58-5] | 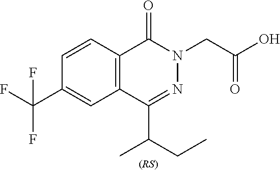 [SMILES: CCC(C)c1nn(CC(=O)O)c(=O)c2ccc(C(F)(F)F)cc12] (I-57) | 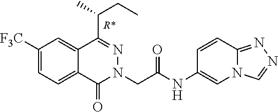 [SMILES: CC[C@@H](C)c1nn(CC(=O)Nc2ccc3nncn3c2)c(=O)c2ccc(C(F)(F)F)cc12] | X24 |
| 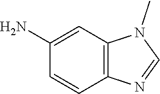 [26530-93-8] | 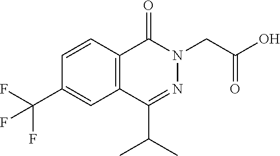 [SMILES: CC(C)c1nn(CC(=O)O)c(=O)c2ccc(C(F)(F)F)cc12] (I-52) | 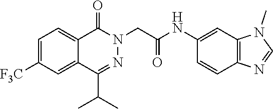 [SMILES: CC(C)c1nn(CC(=O)Nc2ccc3ncn(C)c3c2)c(=O)c2ccc(C(F)(F)F)cc12] | X25 |
| 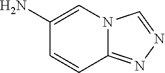 [1087448-58-5] | 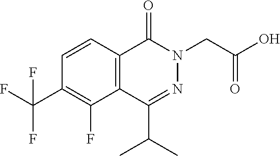 [SMILES: CC(C)c1nn(CC(=O)O)c(=O)c2ccc(C(F)(F)F)c(F)c12] (I-58) | 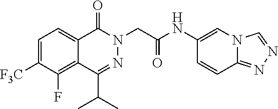 [SMILES: CC(C)c1nn(CC(=O)Nc2ccc3nncn3c2)c(=O)c2ccc(C(F)(F)F)c(F)c12] | X26 |
| 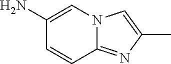 [860258-05-5] | 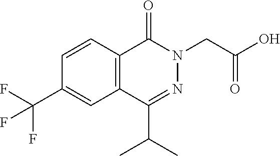 [SMILES: CC(C)c1nn(CC(=O)O)c(=O)c2ccc(C(F)(F)F)cc12] (I-52) | 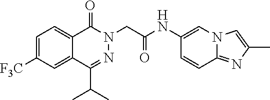 [SMILES: Cc1cn2cc(NC(=O)Cn3nc(C(C)C)c4cc(C(F)(F)F)ccc4c3=O)ccc2n1] | X27 |
| 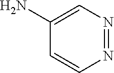 [20744-39-2] | 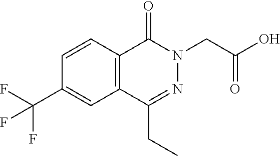 [SMILES: CCc1nn(CC(=O)O)c(=O)c2ccc(C(F)(F)F)cc12] (I-51) | 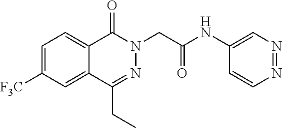 [SMILES: CCc1nn(CC(=O)Nc2ccnnc2)c(=O)c2ccc(C(F)(F)F)cc12] | X30 |
| 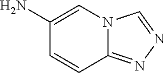 [235106-53-3] | 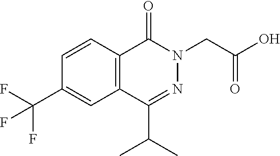 [SMILES: CC(C)c1nn(CC(=O)O)c(=O)c2ccc(C(F)(F)F)cc12] (I-52) | 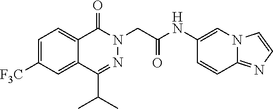 [SMILES: CC(C)c1nn(CC(=O)Nc2ccc3nccn3c2)c(=O)c2ccc(C(F)(F)F)cc12] | X31 |
| 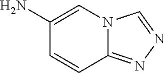 [1087448-58-5] | 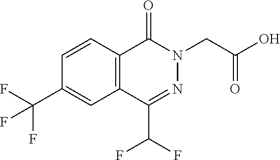 [SMILES: O=C(O)Cn1nc(C(F)F)c2cc(C(F)(F)F)ccc2c1=O] (I-60) | 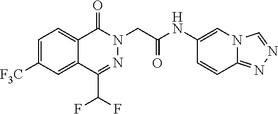 [SMILES: O=C(Cn1nc(C(F)F)c2cc(C(F)(F)F)ccc2c1=O)Nc1ccc2nncn2c1] | X32 |
| 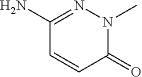 [13506-28-0] | 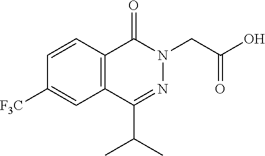 [SMILES: CC(C)c1nn(CC(=O)O)c(=O)c2ccc(C(F)(F)F)cc12] (I-52) | 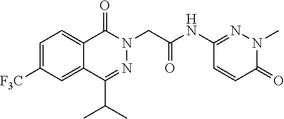 [SMILES: CC(C)c1nn(CC(=O)Nc2ccc(=O)n(C)n2)c(=O)c2ccc(C(F)(F)F)cc12] | X33 |
| 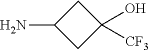 [1251924-07-8] Mixture of diastereomers | 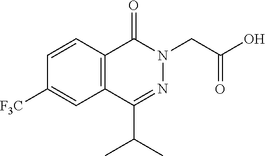 [SMILES: CC(C)c1nn(CC(=O)O)c(=O)c2ccc(C(F)(F)F)cc12] (I-52) | 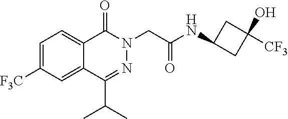 [SMILES: CC(C)c1nn(CC(=O)N[C@H]2C[C@](O)(C(F)(F)F)C2)c(=O)c2ccc(C(F)(F)F)cc12] | X35 |
| 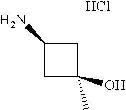 [1523606-23-6] | 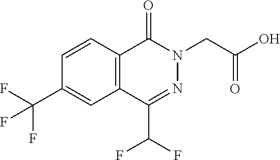 [SMILES: O=C(O)Cn1nc(C(F)F)c2cc(C(F)(F)F)ccc2c1=O] (I-60) | 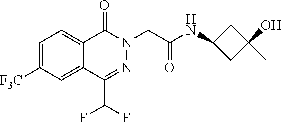 [SMILES: C[C@]1(O)C[C@@H](NC(=O)Cn2nc(C(F)F)c3cc(C(F)(F)F)ccc3c2=O)C1] | X36 |
| 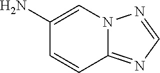 [31052-94-5] | 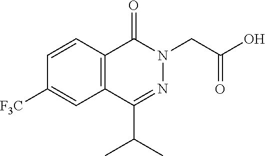 [SMILES: CC(C)c1nn(CC(=O)O)c(=O)c2ccc(C(F)(F)F)cc12] (I-52) | 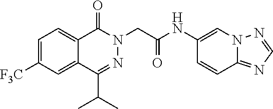 [SMILES: CC(C)c1nn(CC(=O)Nc2ccc3ncnn3c2)c(=O)c2ccc(C(F)(F)F)cc12] | X38 |
| 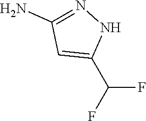 [1284220-49-0] | 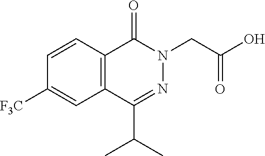 [SMILES: CC(C)c1nn(CC(=O)O)c(=O)c2ccc(C(F)(F)F)cc12] (I-52) | 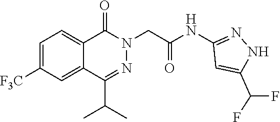 [SMILES: CC(C)c1nn(CC(=O)Nc2cc(C(F)F)[nH]n2)c(=O)c2ccc(C(F)(F)F)cc12] | X39 |
| 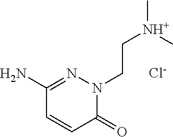 (I-193) | 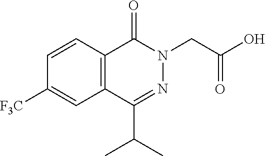 [SMILES: CC(C)c1nn(CC(=O)O)c(=O)c2ccc(C(F)(F)F)cc12] (I-52) | 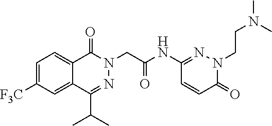 [SMILES: CC(C)c1nn(CC(=O)Nc2ccc(=O)n(CCN(C)C)n2)c(=O)c2ccc(C(F)(F)F)cc12] | X40 |
| 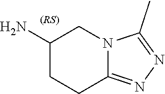 [1251923-84-8] | 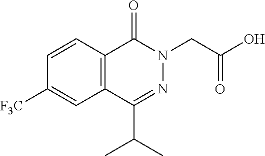 [SMILES: CC(C)c1nn(CC(=O)O)c(=O)c2ccc(C(F)(F)F)cc12] (I-52) | 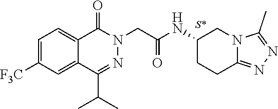 [SMILES: Cc1nnc2n1C[C@@H](NC(=O)Cn1nc(C(C)C)c3cc(C(F)(F)F)ccc3c1=O)CC2] | X41 |
| 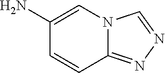 [1087448-58-5] | 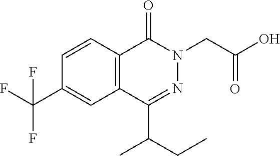 [SMILES: CCC(C)c1nn(CC(=O)O)c(=O)c2ccc(C(F)(F)F)cc12] (I-57) | 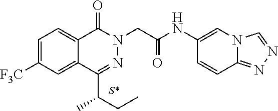 [SMILES: CC[C@H](C)c1nn(CC(=O)Nc2ccc3nncn3c2)c(=O)c2ccc(C(F)(F)F)cc12] | X43 |
| 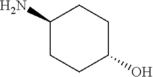 [27489-62-9] | 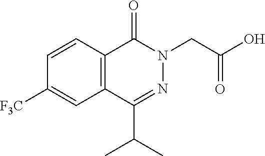 [SMILES: CC(C)c1nn(CC(=O)O)c(=O)c2ccc(C(F)(F)F)cc12] (I-52) | 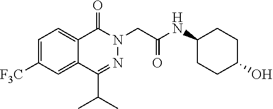 [SMILES: CC(C)c1nn(CC(=O)N[C@H]2CC[C@H](O)CC2)c(=O)c2ccc(C(F)(F)F)cc12] | X44 |
| 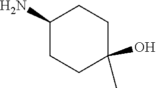 [177906-46-6] | 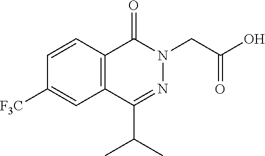 [SMILES: CC(C)c1nn(CC(=O)O)c(=O)c2ccc(C(F)(F)F)cc12] (I-52) | 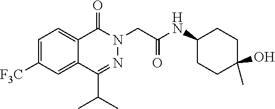 [SMILES: CC(C)c1nn(CC(=O)N[C@H]2CC[C@@](C)(O)CC2)c(=O)c2ccc(C(F)(F)F)cc12] | X46 |
| 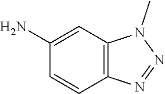 [26861-23-4] | 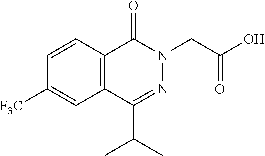 [SMILES: CC(C)c1nn(CC(=O)O)c(=O)c2ccc(C(F)(F)F)cc12] (I-52) | 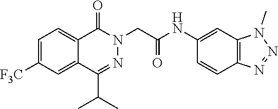 [SMILES: CC(C)c1nn(CC(=O)Nc2ccc3nnn(C)c3c2)c(=O)c2ccc(C(F)(F)F)cc12] | X49 |
| 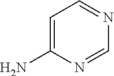 [591-54-8] | 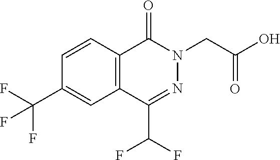 [SMILES: O=C(O)Cn1nc(C(F)F)c2cc(C(F)(F)F)ccc2c1=O] (I-60) | 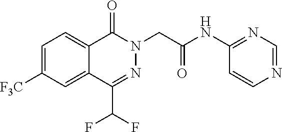 [SMILES: O=C(Cn1nc(C(F)F)c2cc(C(F)(F)F)ccc2c1=O)Nc1ccncn1] | X50 |
| 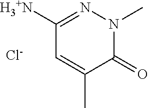 (I-190) | 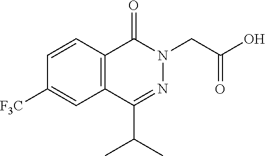 [SMILES: CC(C)c1nn(CC(=O)O)c(=O)c2ccc(C(F)(F)F)cc12] (I-52) | 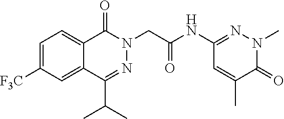 [SMILES: Cc1cc(NC(=O)Cn2nc(C(C)C)c3cc(C(F)(F)F)ccc3c2=O)nn(C)c1=O] | X51 |
| 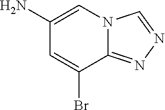 [SMILES: Nc1cc(Br)c2nncn2c1] (I-162) | 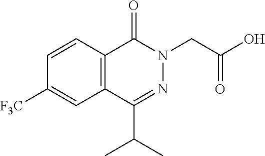 [SMILES: CC(C)c1nn(CC(=O)O)c(=O)c2ccc(C(F)(F)F)cc12] (I-52) | 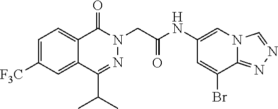 [SMILES: CC(C)c1nn(CC(=O)Nc2cc(Br)c3nncn3c2)c(=O)c2ccc(C(F)(F)F)cc12] | X52 |
| 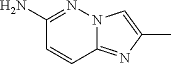 [154704-35-5] | 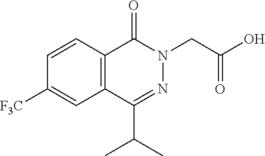 [SMILES: CC(C)c1nn(CC(=O)O)c(=O)c2ccc(C(F)(F)F)cc12] (I-52) | 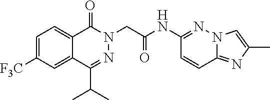 [SMILES: Cc1cn2nc(NC(=O)Cn3nc(C(C)C)c4cc(C(F)(F)F)ccc4c3=O)ccc2n1] | X53 |
| 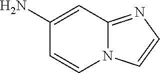 [421595-81-5] | 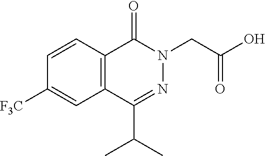 [SMILES: CC(C)c1nn(CC(=O)O)c(=O)c2ccc(C(F)(F)F)cc12] (I-52) | 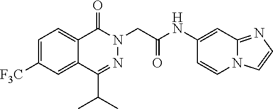 [SMILES: CC(C)c1nn(CC(=O)Nc2ccn3ccnc3c2)c(=O)c2ccc(C(F)(F)F)cc12] | X54 |
| 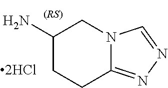 [2260932-36-1] | 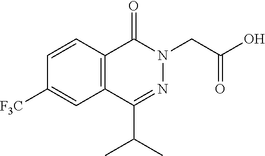 [SMILES: CC(C)c1nn(CC(=O)O)c(=O)c2ccc(C(F)(F)F)cc12] (I-52) | 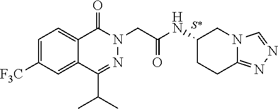 [SMILES: CC(C)c1nn(CC(=O)N[C@H]2CCc3nncn3C2)c(=O)c2ccc(C(F)(F)F)cc12] | X55 |
| 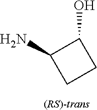 [1609406-69-0] | 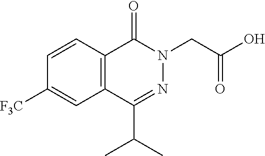 [SMILES: CC(C)c1nn(CC(=O)O)c(=O)c2ccc(C(F)(F)F)cc12] (I-52) | 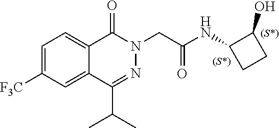 [SMILES: CC(C)c1nn(CC(=O)N[C@H]2CC[C@@H]2O)c(=O)c2ccc(C(F)(F)F)cc12] | X56 |
| 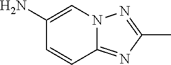 [7169-94-0] | 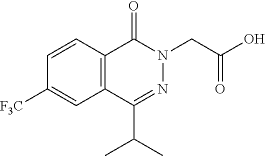 [SMILES: CC(C)c1nn(CC(=O)O)c(=O)c2ccc(C(F)(F)F)cc12] (I-52) | 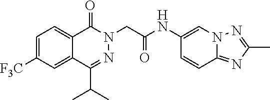 [SMILES: Cc1nc2ccc(NC(=O)Cn3nc(C(C)C)c4cc(C(F)(F)F)ccc4c3=O)cn2n1] | X57 |
| 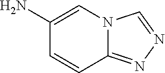 [1087448-58-5] | 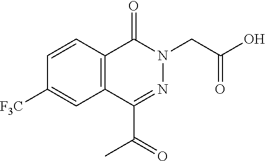 [SMILES: CC(=O)c1nn(CC(=O)O)c(=O)c2ccc(C(F)(F)F)cc12] (I-61) | 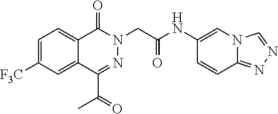 [SMILES: CC(=O)c1nn(CC(=O)Nc2ccc3nncn3c2)c(=O)c2ccc(C(F)(F)F)cc12] | X58 |
| 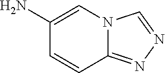 [1087448-58-5] | 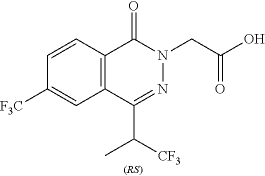 [SMILES: CC(c1nn(CC(=O)O)c(=O)c2ccc(C(F)(F)F)cc12)C(F)(F)F] (I-62) | 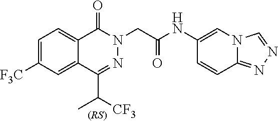 [SMILES: CC(c1nn(CC(=O)Nc2ccc3nncn3c2)c(=O)c2ccc(C(F)(F)F)cc12)C(F)(F)F] | X59 |
| 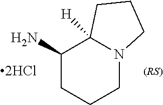 [MFCD28892924] | 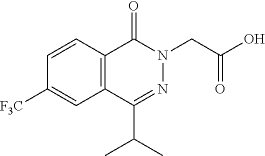 [SMILES: CC(C)c1nn(CC(=O)O)c(=O)c2ccc(C(F)(F)F)cc12] (I-52) | 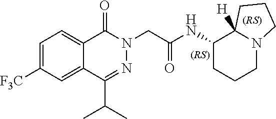 [SMILES: CC(C)c1nn(CC(=O)N[C@H]2CCCN3CCC[C@@H]23)c(=O)c2ccc(C(F)(F)F)cc12] | X60 |
| 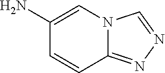 [1087448-58-5] | 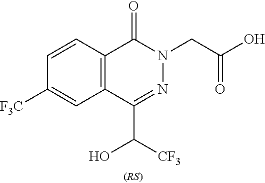 [SMILES: O=C(O)Cn1nc(C(O)C(F)(F)F)c2cc(C(F)(F)F)ccc2c1=O] (I-63) | 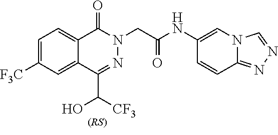 [SMILES: O=C(Cn1nc(C(O)C(F)(F)F)c2cc(C(F)(F)F)ccc2c1=O)Nc1ccc2nncn2c1] | X61 |
| 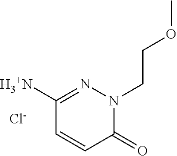 (I-187) | 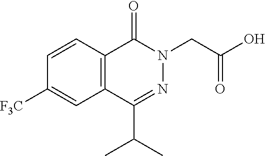 [SMILES: CC(C)c1nn(CC(=O)O)c(=O)c2ccc(C(F)(F)F)cc12] (I-52) | 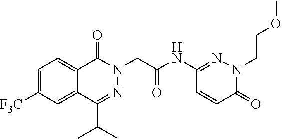 [SMILES: COCCn1nc(NC(=O)Cn2nc(C(C)C)c3cc(C(F)(F)F)ccc3c2=O)ccc1=O] | X62 |
| 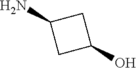 [1036260-43-1] | 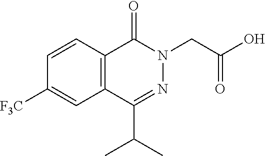 [SMILES: CC(C)c1nn(CC(=O)O)c(=O)c2ccc(C(F)(F)F)cc12] (I-52) | 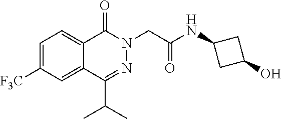 [SMILES: CC(C)c1nn(CC(=O)N[C@H]2C[C@@H](O)C2)c(=O)c2ccc(C(F)(F)F)cc12] | X67 |
| 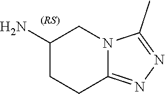 [1251923-84-8] | 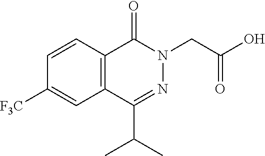 [SMILES: CC(C)c1nn(CC(=O)O)c(=O)c2ccc(C(F)(F)F)cc12] (I-52) | 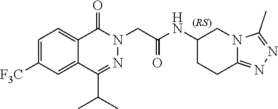 [SMILES: Cc1nnc2n1CC(NC(=O)Cn1nc(C(C)C)c3cc(C(F)(F)F)ccc3c1=O)CC2] | X68 |
| 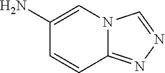 [1087448-58-5] | 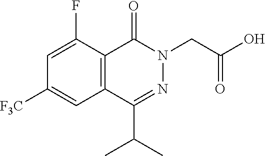 [SMILES: CC(C)c1nn(CC(=O)O)c(=O)c2c(F)cc(C(F)(F)F)cc12] (I-64) | 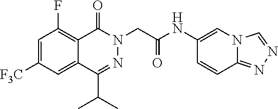 [SMILES: CC(C)c1nn(CC(=O)Nc2ccc3nncn3c2)c(=O)c2c(F)cc(C(F)(F)F)cc12] | X71 |
| 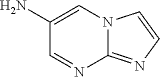 [944900-19-0] | 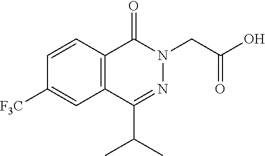 [SMILES: CC(C)c1nn(CC(=O)O)c(=O)c2ccc(C(F)(F)F)cc12] (I-52) | 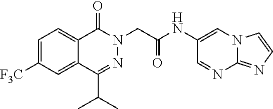 [SMILES: CC(C)c1nn(CC(=O)Nc2cnc3nccn3c2)c(=O)c2ccc(C(F)(F)F)cc12] | X73 |
| 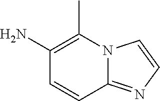 [573764-90-6] | 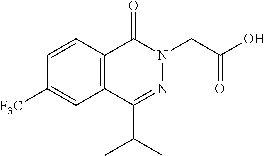 [SMILES: CC(C)c1nn(CC(=O)O)c(=O)c2ccc(C(F)(F)F)cc12] (I-52) | 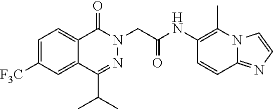 [SMILES: Cc1c(NC(=O)Cn2nc(C(C)C)c3cc(C(F)(F)F)ccc3c2=O)ccc2nccn12] | X74 |
| 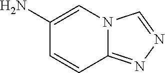 [1087448-58-5] | 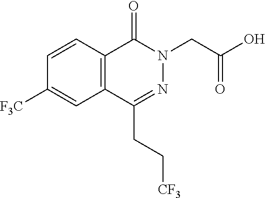 [SMILES: O=C(O)Cn1nc(CCC(F)(F)F)c2cc(C(F)(F)F)ccc2c1=O] (I-65) | 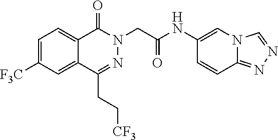 [SMILES: O=C(Cn1nc(CCC(F)(F)F)c2cc(C(F)(F)F)ccc2c1=O)Nc1ccc2nncn2c1] | X76 |
| 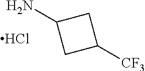 cis/trans mixture [1803601-06-0] | 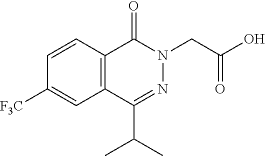 [SMILES: CC(C)c1nn(CC(=O)O)c(=O)c2ccc(C(F)(F)F)cc12] (I-52) | 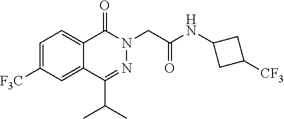 [SMILES: CC(C)c1nn(CC(=O)NC2CC(C(F)(F)F)C2)c(=O)c2ccc(C(F)(F)F)cc12] cis/trans mixture | X77 |
| 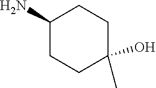 [177908-37-1] | 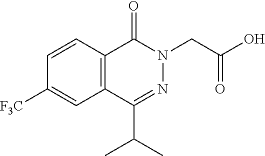 [SMILES: CC(C)c1nn(CC(=O)O)c(=O)c2ccc(C(F)(F)F)cc12] (I-52) | 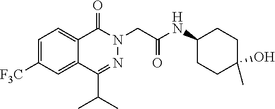 [SMILES: CC(C)c1nn(CC(=O)N[C@H]2CC[C@](C)(O)CC2)c(=O)c2ccc(C(F)(F)F)cc12] | X78 |
| 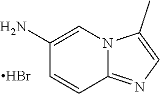 [1803596-49-7] | 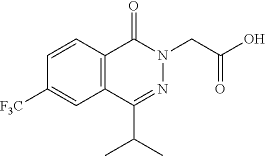 [SMILES: CC(C)c1nn(CC(=O)O)c(=O)c2ccc(C(F)(F)F)cc12] (I-52) | 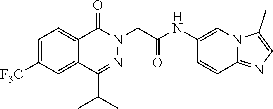 [SMILES: Cc1cnc2ccc(NC(=O)Cn3nc(C(C)C)c4cc(C(F)(F)F)ccc4c3=O)cn12] | X79 |
| 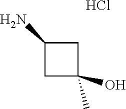 [1523606-23-6] | 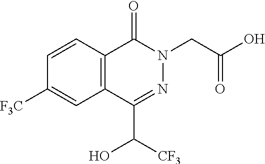 [SMILES: O=C(O)Cn1nc(C(O)C(F)(F)F)c2cc(C(F)(F)F)ccc2c1=O] (I-63) | 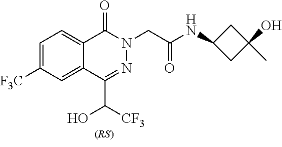 [SMILES: C[C@]1(O)C[C@@H](NC(=O)Cn2nc(C(O)C(F)(F)F)c3cc(C(F)(F)F)ccc3c2=O)C1] | X84 |
| 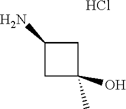 [1523606-23-6] | 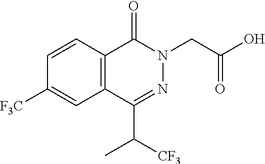 [SMILES: CC(c1nn(CC(=O)O)c(=O)c2ccc(C(F)(F)F)cc12)C(F)(F)F] (I-62) | 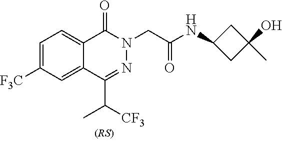 [SMILES: CC(c1nn(CC(=O)N[C@H]2C[C@@](C)(O)C2)c(=O)c2ccc(C(F)(F)F)cc12)C(F)(F)F] | X85 |
| 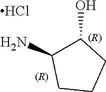 [68327-11-7] | 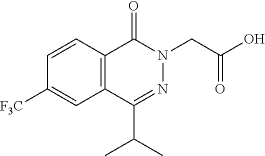 [SMILES: CC(C)c1nn(CC(=O)O)c(=O)c2ccc(C(F)(F)F)cc12] (I-52) | 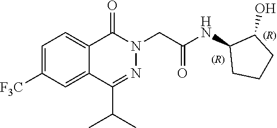 | X86 |
| 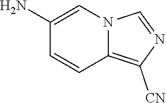 [SMILES: N#Cc1ncn2cc(N)ccc12] 1-181 | 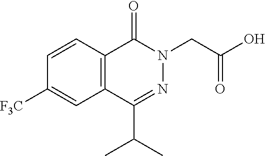 [SMILES: CC(C)c1nn(CC(=O)O)c(=O)c2ccc(C(F)(F)F)cc12] (I-52) | 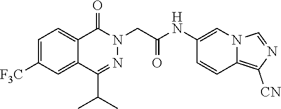 [SMILES: CC(C)c1nn(CC(=O)Nc2ccc3c(C#N)ncn3c2)c(=O)c2ccc(C(F)(F)F)cc12] | X88 |
| 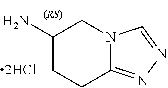 [2260932-36-1] | 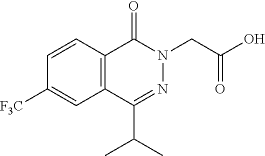 [SMILES: CC(C)c1nn(CC(=O)O)c(=O)c2ccc(C(F)(F)F)cc12] (I-52) | 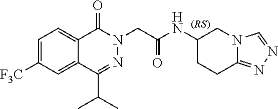 [SMILES: CC(C)c1nn(CC(=O)NC2CCc3nncn3C2)c(=O)c2ccc(C(F)(F)F)cc12] | X89 |
| 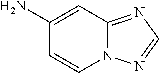 [1396312-30-3] | 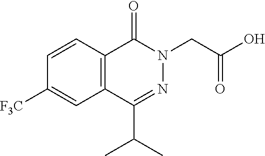 [SMILES: CC(C)c1nn(CC(=O)O)c(=O)c2ccc(C(F)(F)F)cc12] (I-52) | 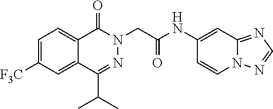 [SMILES: CC(C)c1nn(CC(=O)Nc2ccn3ncnc3c2)c(=O)c2ccc(C(F)(F)F)cc12] | X90 |
| 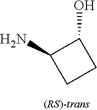 [1609406-69-0] | 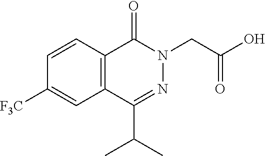 [SMILES: CC(C)c1nn(CC(=O)O)c(=O)c2ccc(C(F)(F)F)cc12] (I-52) | 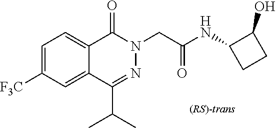 [SMILES: CC(C)c1nn(CC(=O)N[C@H]2CC[C@@H]2O)c(=O)c2ccc(C(F)(F)F)cc12] | X91 |
| 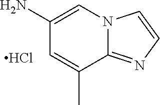 [1803591-03-8] | 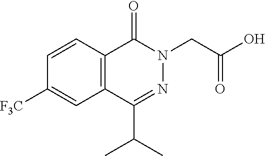 [SMILES: CC(C)c1nn(CC(=O)O)c(=O)c2ccc(C(F)(F)F)cc12] (I-52) | 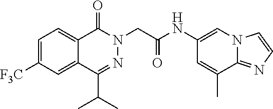 [SMILES: Cc1cc(NC(=O)Cn2nc(C(C)C)c3cc(C(F)(F)F)ccc3c2=O)cn2ccnc12] | X92 |
| 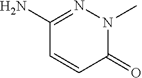 [13506-28-0] | 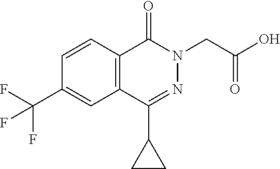 [SMILES: O=C(O)Cn1nc(C2CC2)c2cc(C(F)(F)F)ccc2c1=O] (I-59) | 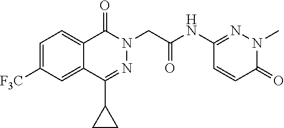 [SMILES: Cn1nc(NC(=O)Cn2nc(C3CC3)c3cc(C(F)(F)F)ccc3c2=O)ccc1=O] | X93 |
| 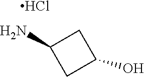 [1205037-95-1] | 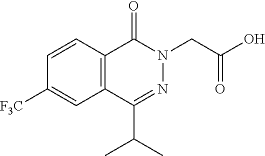 [SMILES: CC(C)c1nn(CC(=O)O)c(=O)c2ccc(C(F)(F)F)cc12] (I-52) | 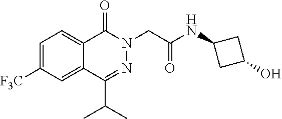 [SMILES: CC(C)c1nn(CC(=O)N[C@H]2C[C@H](O)C2)c(=O)c2ccc(C(F)(F)F)cc12] | X94 |
| 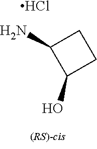 [2070860-49-8] | 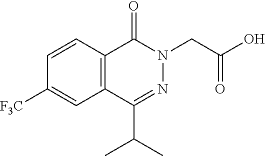 [SMILES: CC(C)c1nn(CC(=O)O)c(=O)c2ccc(C(F)(F)F)cc12] (I-52) | 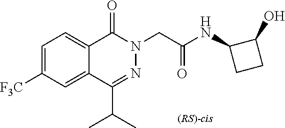 [SMILES: CC(C)c1nn(CC(=O)N[C@@H]2CC[C@@H]2O)c(=O)c2ccc(C(F)(F)F)cc12] | X95 |
| 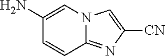 [SMILES: N#Cc1cn2cc(N)ccc2n1] (I-178) | 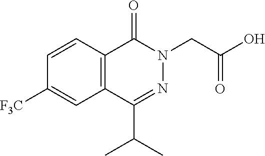 [SMILES: CC(C)c1nn(CC(=O)O)c(=O)c2ccc(C(F)(F)F)cc12] (I-52) | 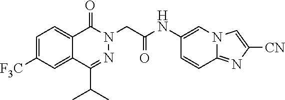 [SMILES: CC(C)c1nn(CC(=O)Nc2ccc3nc(C#N)cn3c2)c(=O)c2ccc(C(F)(F)F)cc12] | X97 |
| 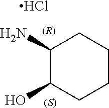 [2070860-49-8] | 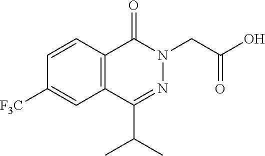 [SMILES: CC(C)c1nn(CC(=O)O)c(=O)c2ccc(C(F)(F)F)cc12] (I-52) | 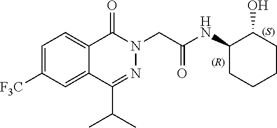 | X98 |
| 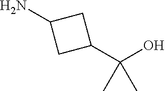 Cis/trans mixture [1609546-13-5] | 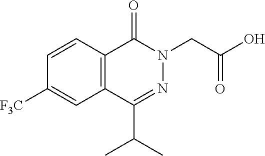 [SMILES: CC(C)c1nn(CC(=O)O)c(=O)c2ccc(C(F)(F)F)cc12] (I-52) | 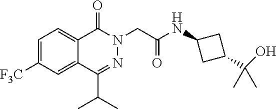 [SMILES: CC(C)c1nn(CC(=O)N[C@H]2C[C@H](C(C)(C)O)C2)c(=O)c2ccc(C(F)(F)F)cc12] | X99 |
| 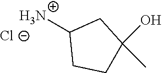 (RS)-cis/trans mixture I-177 | 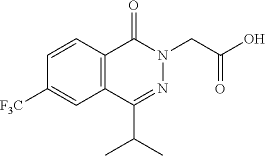 [SMILES: CC(C)c1nn(CC(=O)O)c(=O)c2ccc(C(F)(F)F)cc12] (I-52) | 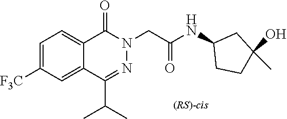 [SMILES: CC(C)c1nn(CC(=O)N[C@@H]2CC[C@](C)(O)C2)c(=O)c2ccc(C(F)(F)F)cc12] | X100 |
| 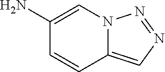 (I-173) | 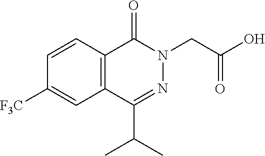 [SMILES: CC(C)c1nn(CC(=O)O)c(=O)c2ccc(C(F)(F)F)cc12] (I-52) | 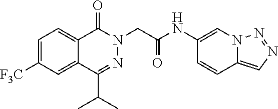 [SMILES: CC(C)c1nn(CC(=O)Nc2ccc3cnnn3c2)c(=O)c2ccc(C(F)(F)F)cc12] | X101 |
| 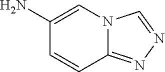 [1087448-58-5] | 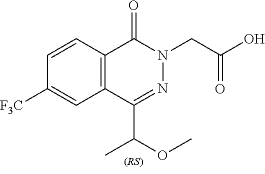 [SMILES: COC(C)c1nn(CC(=O)O)c(=O)c2ccc(C(F)(F)F)cc12] (I-66) | 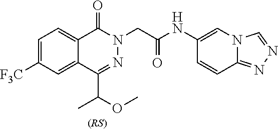 [SMILES: COC(C)c1nn(CC(=O)Nc2ccc3nncn3c2)c(=O)c2ccc(C(F)(F)F)cc12] | X102 |
| 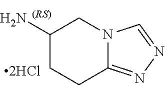 [2260932-36-1] | 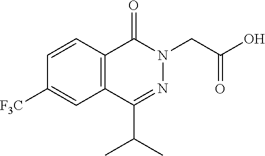 [SMILES: CC(C)c1nn(CC(=O)O)c(=O)c2ccc(C(F)(F)F)cc12] (I-52) | 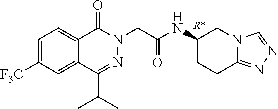 [SMILES: CC(C)c1nn(CC(=O)N[C@@H]2CCc3nncn3C2)c(=O)c2ccc(C(F)(F)F)cc12] | X106 |
| 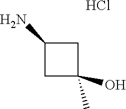 [1523606-23-6] | 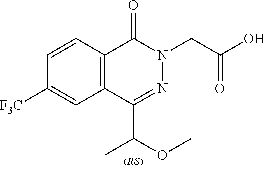 [SMILES: COC(C)c1nn(CC(=O)O)c(=O)c2ccc(C(F)(F)F)cc12] (I-66) | 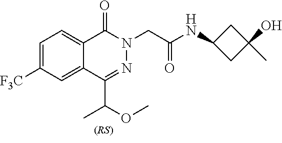 [SMILES: COC(C)c1nn(CC(=O)N[C@H]2C[C@@](C)(O)C2)c(=O)c2ccc(C(F)(F)F)cc12] | X107 |
| 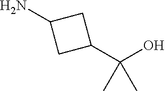 Cis/trans mixture [1609546-13-5] | 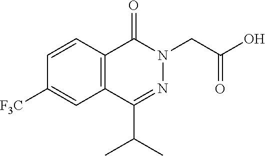 [SMILES: CC(C)c1nn(CC(=O)O)c(=O)c2ccc(C(F)(F)F)cc12] (I-52) | 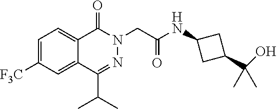 [SMILES: CC(C)c1nn(CC(=O)N[C@H]2C[C@@H](C(C)(C)O)C2)c(=O)c2ccc(C(F)(F)F)cc12] | X108 |
| 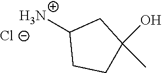 (RS)-cis/trans mixture I-177 | 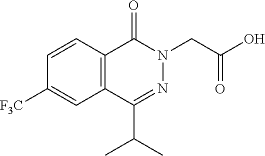 [SMILES: CC(C)c1nn(CC(=O)O)c(=O)c2ccc(C(F)(F)F)cc12] (I-52) | 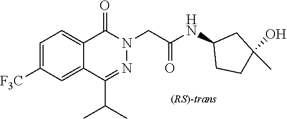 [SMILES: CC(C)c1nn(CC(=O)N[C@@H]2CC[C@@](C)(O)C2)c(=O)c2ccc(C(F)(F)F)cc12] | X110 |
| 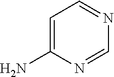 [591-54-8 | 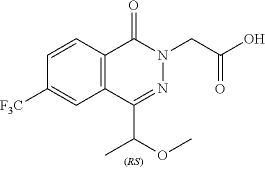 [SMILES: COC(C)c1nn(CC(=O)O)c(=O)c2ccc(C(F)(F)F)cc12] (I-66) | 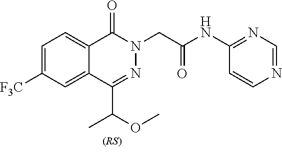 [SMILES: COC(C)c1nn(CC(=O)Nc2ccncn2)c(=O)c2ccc(C(F)(F)F)cc12] | X111 |
| 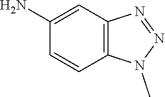 [27799-83-3] | 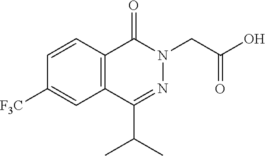 [SMILES: CC(C)c1nn(CC(=O)O)c(=O)c2ccc(C(F)(F)F)cc12] (I-52) | 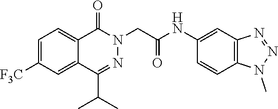 [SMILES: CC(C)c1nn(CC(=O)Nc2ccc3c(c2)nnn3C)c(=O)c2ccc(C(F)(F)F)cc12] | X113 |
| 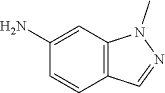 [74728-65-7] | 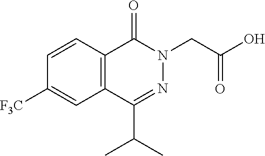 [SMILES: CC(C)c1nn(CC(=O)O)c(=O)c2ccc(C(F)(F)F)cc12] (I-52) | 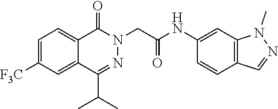 [SMILES: CC(C)c1nn(CC(=O)Nc2ccc3cnn(C)c3c2)c(=O)c2ccc(C(F)(F)F)cc12] | X114 |
| 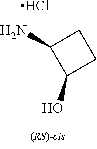 [2070860-49-8] | 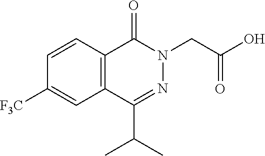 [SMILES: CC(C)c1nn(CC(=O)O)c(=O)c2ccc(C(F)(F)F)cc12] (I-52) | 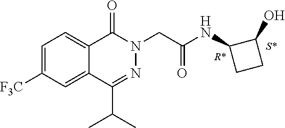 [SMILES: CC(C)c1nn(CC(=O)N[C@@H]2CC[C@@H]2O)c(=O)c2ccc(C(F)(F)F)cc12] | X115 |
| 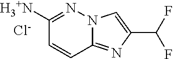 (I-169) | 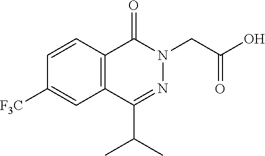 [SMILES: CC(C)c1nn(CC(=O)O)c(=O)c2ccc(C(F)(F)F)cc12] (I-52) | 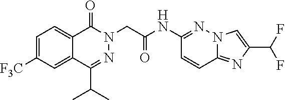 [SMILES: CC(C)c1nn(CC(=O)Nc2ccc3nc(C(F)F)cn3n2)c(=O)c2ccc(C(F)(F)F)cc12] | X118 |
| 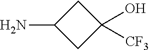 [1251924-07-8] Mixture of diastereomers | 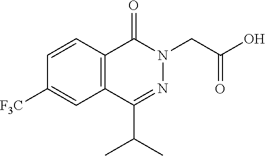 [SMILES: CC(C)c1nn(CC(=O)O)c(=O)c2ccc(C(F)(F)F)cc12] (I-52) | 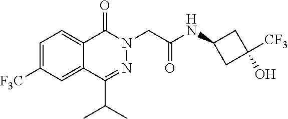 [SMILES: CC(C)c1nn(CC(=O)N[C@H]2C[C@@](O)(C(F)(F)F)C2)c(=O)c2ccc(C(F)(F)F)cc12] | X119 |
| 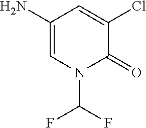 [SMILES: Nc1cc(Cl)c(=O)n(C(F)F)c1] (I-167) | 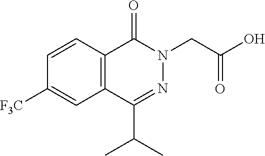 [SMILES: CC(C)c1nn(CC(=O)O)c(=O)c2ccc(C(F)(F)F)cc12] (I-52) | 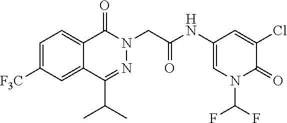 [SMILES: CC(C)c1nn(CC(=O)Nc2cc(Cl)c(=O)n(C(F)F)c2)c(=O)c2ccc(C(F)(F)F)cc12] | X120 |
| 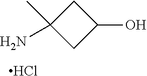 [1403766-64-2] Mixture of diastereomers | 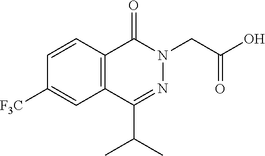 [SMILES: CC(C)c1nn(CC(=O)O)c(=O)c2ccc(C(F)(F)F)cc12] (I-52) | 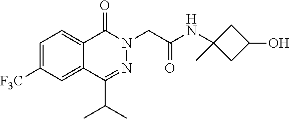 [SMILES: CC(C)c1nn(CC(=O)NC2(C)CC(O)C2)c(=O)c2ccc(C(F)(F)F)cc12] Cis/trans mixture | X121 |
| 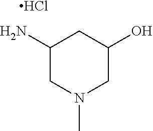 Cis/trans mixture (1-163) | 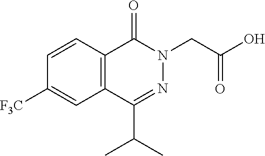 [SMILES: CC(C)c1nn(CC(=O)O)c(=O)c2ccc(C(F)(F)F)cc12] (I-52) | 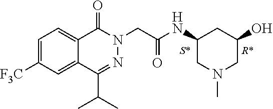 [SMILES: CC(C)c1nn(CC(=O)N[C@H]2C[C@@H](O)CN(C)C2)c(=O)c2ccc(C(F)(F)F)cc12] | XI22 |
| 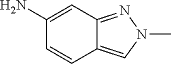 [50593-30-1] | 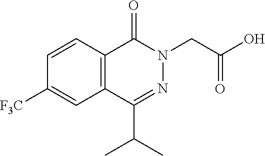 [SMILES: CC(C)c1nn(CC(=O)O)c(=O)c2ccc(C(F)(F)F)cc12] (I-52) | 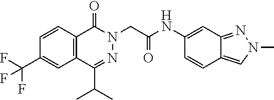 [SMILES: CC(C)c1nn(CC(=O)Nc2ccc3cn(C)nc3c2)c(=O)c2ccc(C(F)(F)F)cc12] | X123 |
| 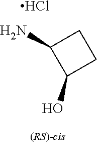 [2070860-49-8] | 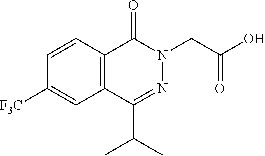 [SMILES: CC(C)c1nn(CC(=O)O)c(=O)c2ccc(C(F)(F)F)cc12] (I-52) | 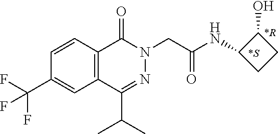 [SMILES: CC(C)c1nn(CC(=O)N[C@H]2CC[C@H]2O)c(=O)c2ccc(C(F)(F)F)cc12] | X124 |
| 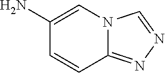 [1087448-58-5] | 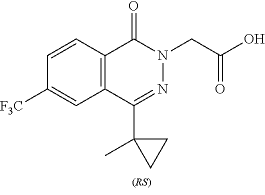 [SMILES: CC1(c2nn(CC(=O)O)c(=O)c3ccc(C(F)(F)F)cc23)CC1] (I-67) | 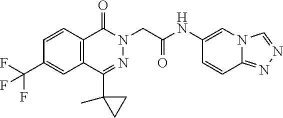 [SMILES: CC1(c2nn(CC(=O)Nc3ccc4nncn4c3)c(=O)c3ccc(C(F)(F)F)cc23)CC1] | X125 |
| 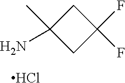 [1408076-03-8] | 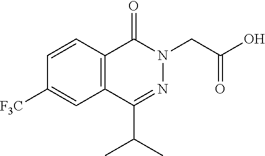 [SMILES: CC(C)c1nn(CC(=O)O)c(=O)c2ccc(C(F)(F)F)cc12] (I-52) | 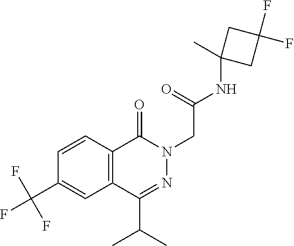 [SMILES: CC(C)c1nn(CC(=O)NC2(C)CC(F)(F)C2)c(=O)c2ccc(C(F)(F)F)cc12] | X126 |
| 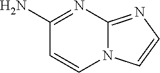 [462651-80-5] | 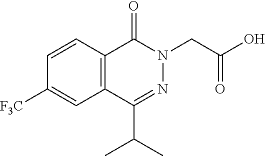 [SMILES: CC(C)c1nn(CC(=O)O)c(=O)c2ccc(C(F)(F)F)cc12] (I-52) | 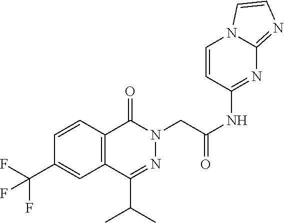 [SMILES: CC(C)c1nn(CC(=O)Nc2ccn3ccnc3n2)c(=O)c2ccc(C(F)(F)F)cc12] | X127 |
| 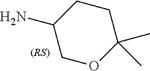 [1785762-88-0] | 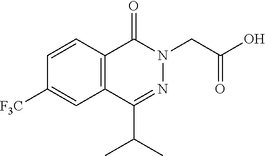 [SMILES: CC(C)c1nn(CC(=O)O)c(=O)c2ccc(C(F)(F)F)cc12] (I-52) | 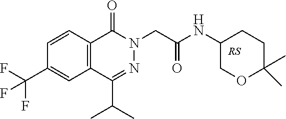 [SMILES: CC(C)c1nn(CC(=O)NC2CCC(C)(C)OC2)c(=O)c2ccc(C(F)(F)F)cc12] | X128 |
| 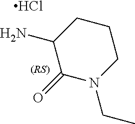 [2126160-37-8] | 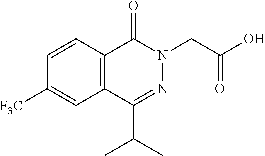 [SMILES: CC(C)c1nn(CC(=O)O)c(=O)c2ccc(C(F)(F)F)cc12] (I-52) | 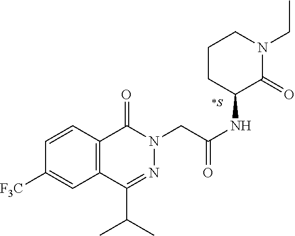 [SMILES: CCN1CCC[C@H](NC(=O)Cn2nc(C(C)C)c3cc(C(F)(F)F)ccc3c2=O)C1=O] | X129 |
| 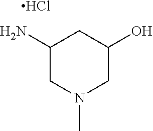 Cis/trans mixture (I-163) | 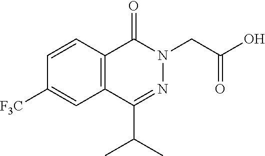 [SMILES: CC(C)c1nn(CC(=O)O)c(=O)c2ccc(C(F)(F)F)cc12] (I-52) | 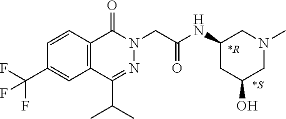 [SMILES: CC(C)c1nn(CC(=O)N[C@@H]2C[C@H](O)CN(C)C2)c(=O)c2ccc(C(F)(F)F)cc12] | X130 |
| 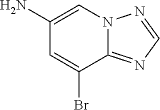 [SMILES: Nc1cc(Br)c2ncnn2c1] (I-159) | 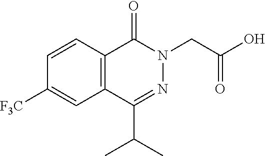 [SMILES: CC(C)c1nn(CC(=O)O)c(=O)c2ccc(C(F)(F)F)cc12] (I-52) | 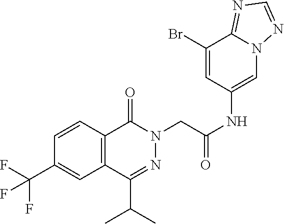 [SMILES: CC(C)c1nn(CC(=O)Nc2cc(Br)c3ncnn3c2)c(=O)c2ccc(C(F)(F)F)cc12] | X132 |
| 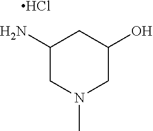 Cis/trans mixture (I-163) | 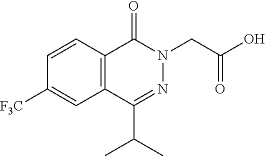 [SMILES: CC(C)c1nn(CC(=O)O)c(=O)c2ccc(C(F)(F)F)cc12] (I-52) | 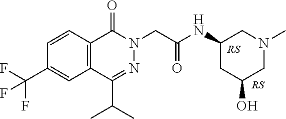 [SMILES: CC(C)c1nn(CC(=O)N[C@@H]2C[C@H](O)CN(C)C2)c(=O)c2ccc(C(F)(F)F)cc12] | X133 |
| 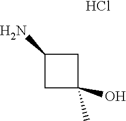 [1523606-23-6] | 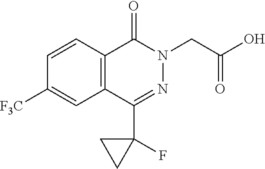 [SMILES: O=C(O)Cn1nc(C2(F)CC2)c2cc(C(F)(F)F)ccc2c1=O] (I-68) | 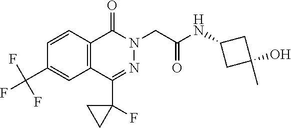 [SMILES: C[C@]1(O)C[C@@H](NC(=O)Cn2nc(C3(F)CC3)c3cc(C(F)(F)F)ccc3c2=O)C1] | X134 |
| 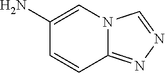 [1087448-58-5] | 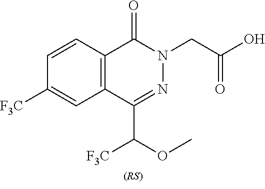 [SMILES: COC(c1nn(CC(=O)O)c(=O)c2ccc(C(F)(F)F)cc12)C(F)(F)F] (I-69) | 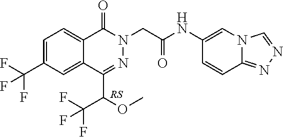 [SMILES: COC(c1nn(CC(=O)Nc2ccc3nncn3c2)c(=O)c2ccc(C(F)(F)F)cc12)C(F)(F)F] | X135 |
| 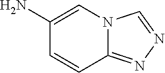 [1087448-58-5] | 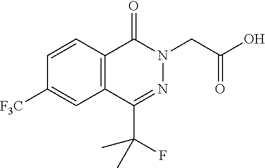 [SMILES: CC(C)(F)c1nn(CC(=O)O)c(=O)c2ccc(C(F)(F)F)cc12] (I-70) | 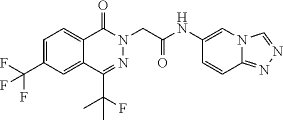 [SMILES: CC(C)(F)c1nn(CC(=O)Nc2ccc3nncn3c2)c(=O)c2ccc(C(F)(F)F)cc12] | X136 |
| 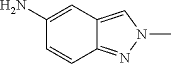 (I-158) | 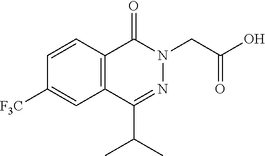 [SMILES: CC(C)c1nn(CC(=O)O)c(=O)c2ccc(C(F)(F)F)cc12] (I-52) | 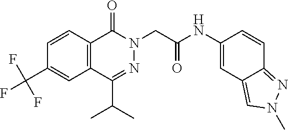 [SMILES: CC(C)c1nn(CC(=O)Nc2ccc3nn(C)cc3c2)c(=O)c2ccc(C(F)(F)F)cc12] | X137 |
| 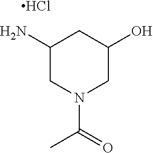 Cis/trans mixture (I-165) | 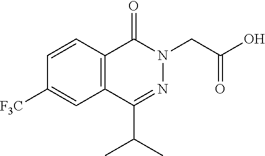 [SMILES: CC(C)c1nn(CC(=O)O)c(=O)c2ccc(C(F)(F)F)cc12] (I-52) | 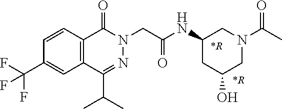 [SMILES: CC(=O)N1C[C@H](O)C[C@@H](NC(=O)Cn2nc(C(C)C)c3cc(C(F)(F)F)ccc3c2=O)C1] | X138 |
| 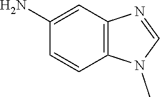 [10394-38-4] | 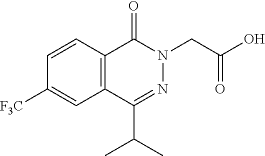 [SMILES: CC(C)c1nn(CC(=O)O)c(=O)c2ccc(C(F)(F)F)cc12] (I-52) | 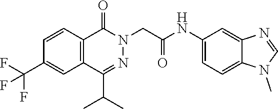 [SMILES: CC(C)c1nn(CC(=O)Nc2ccc3c(c2)ncn3C)c(=O)c2ccc(C(F)(F)F)cc12] | X141 |
| 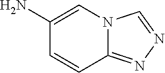 [1087448-58-5] | 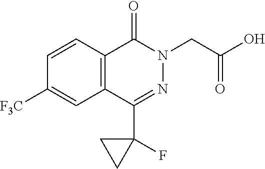 [SMILES: O=C(O)Cn1nc(C2(F)CC2)c2cc(C(F)(F)F)ccc2c1=O] (I-68) | 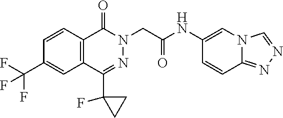 [SMILES: O=C(Cn1nc(C2(F)CC2)c2cc(C(F)(F)F)ccc2c1=O)Nc1ccc2nncn2c1] | X142 |
| 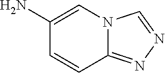 [1087448-58-5] | 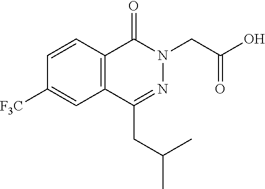 [SMILES: CC(C)Cc1nn(CC(=O)O)c(=O)c2ccc(C(F)(F)F)cc12] (I-71) | 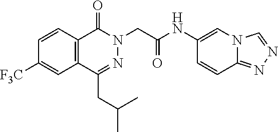 [SMILES: CC(C)Cc1nn(CC(=O)Nc2ccc3nncn3c2)c(=O)c2ccc(C(F)(F)F)cc12] | X144 |
| 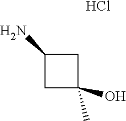 [1523606-23-6] | 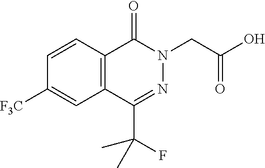 [SMILES: CC(C)(F)c1nn(CC(=O)O)c(=O)c2ccc(C(F)(F)F)cc12] (I-70) | 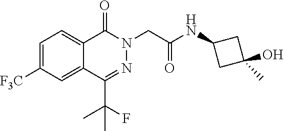 [SMILES: CC(C)(F)c1nn(CC(=O)N[C@H]2C[C@@](C)(O)C2)c(=O)c2ccc(C(F)(F)F)cc12] | X145 |
| 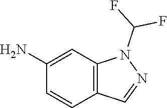 [SMILES: Nc1ccc2cnn(C(F)F)c2c1] I-156 | 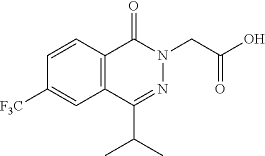 [SMILES: CC(C)c1nn(CC(=O)O)c(=O)c2ccc(C(F)(F)F)cc12] (I-52) | 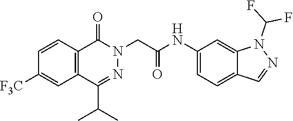 [SMILES: CC(C)c1nn(CC(=O)Nc2ccc3cnn(C(F)F)c3c2)c(=O)c2ccc(C(F)(F)F)cc12] | X146 |
| 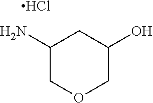 Cis/trans mixture [2102408-50-2] | 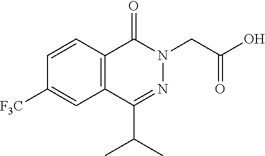 [SMILES: CC(C)c1nn(CC(=O)O)c(=O)c2ccc(C(F)(F)F)cc12] (I-52) | 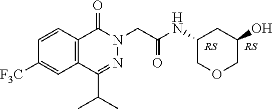 [SMILES: CC(C)c1nn(CC(=O)N[C@H]2COC[C@H](O)C2)c(=O)c2ccc(C(F)(F)F)cc12] | X148 |
| 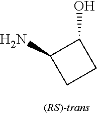 [1609406-69-0] | 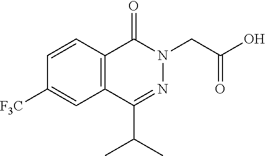 [SMILES: CC(C)c1nn(CC(=O)O)c(=O)c2ccc(C(F)(F)F)cc12] (I-52) | 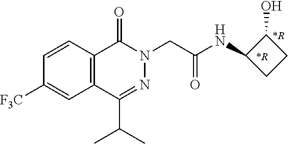 [SMILES: CC(C)c1nn(CC(=O)N[C@@H]2CC[C@H]2O)c(=O)c2ccc(C(F)(F)F)cc12] | X149 |
| 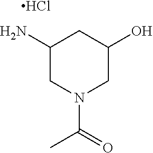 Cis/trans mixture (1-165) | 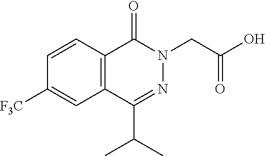 [SMILES: CC(C)c1nn(CC(=O)O)c(=O)c2ccc(C(F)(F)F)cc12] (I-52) | 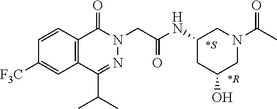 [SMILES: CC(=O)N1C[C@H](O)C[C@H](NC(=O)Cn2nc(C(C)C)c3cc(C(F)(F)F)ccc3c2=O)C1] | X150 |
| 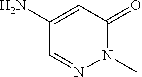 [13506-27-9] | 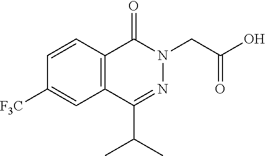 [SMILES: CC(C)c1nn(CC(=O)O)c(=O)c2ccc(C(F)(F)F)cc12] (I-52) | 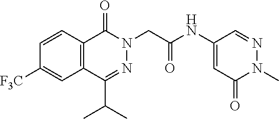 [SMILES: CC(C)c1nn(CC(=O)Nc2cnn(C)c(=O)c2)c(=O)c2ccc(C(F)(F)F)cc12] | X151 |
| 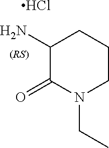 [2126160-37-8] | 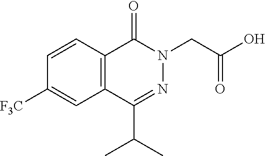 [SMILES: CC(C)c1nn(CC(=O)O)c(=O)c2ccc(C(F)(F)F)cc12] (I-52) | 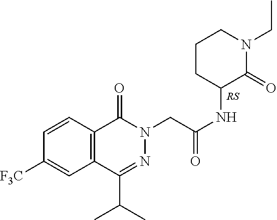 [SMILES: CCN1CCCC(NC(=O)Cn2nc(C(C)C)c3cc(C(F)(F)F)ccc3c2=O)C1=O] | X152 |
| 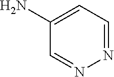 [20744-39-2] | 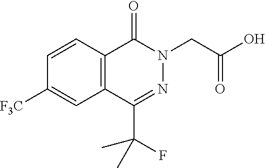 [SMILES: CC(C)(F)c1nn(CC(=O)O)c(=O)c2ccc(C(F)(F)F)cc12] (I-70) | 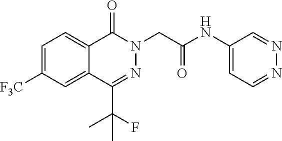 [SMILES: CC(C)(F)c1nn(CC(=O)Nc2ccnnc2)c(=O)c2ccc(C(F)(F)F)cc12] | X155 |
| 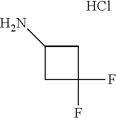 [637031-93-7] | 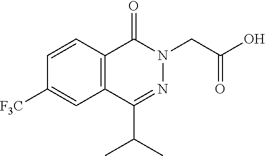 [SMILES: CC(C)c1nn(CC(=O)O)c(=O)c2ccc(C(F)(F)F)cc12] (I-52) | 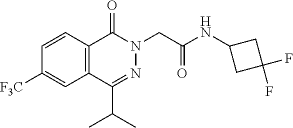 [SMILES: CC(C)c1nn(CC(=O)NC2CC(F)(F)C2)c(=O)c2ccc(C(F)(F)F)cc12] | X156 |
| 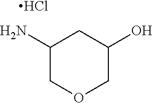 Cis/trans mixture [2102408-50-2] | 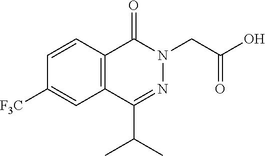 [SMILES: CC(C)c1nn(CC(=O)O)c(=O)c2ccc(C(F)(F)F)cc12] (I-52) | 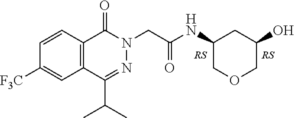 [SMILES: CC(C)c1nn(CC(=O)N[C@@H]2COC[C@H](O)C2)c(=O)c2ccc(C(F)(F)F)cc12] | X157 |
| 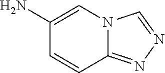 [1087448-58-5] | 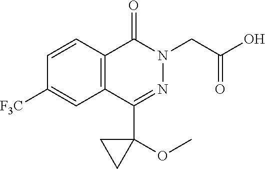 [SMILES: COC1(c2nn(CC(=O)O)c(=O)c3ccc(C(F)(F)F)cc23)CC1] (I-72) | 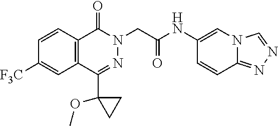 [SMILES: COC1(c2nn(CC(=O)Nc3ccc4nncn4c3)c(=O)c3ccc(C(F)(F)F)cc23)CC1] | X158 |
| 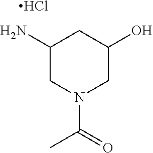 Cis/trans mixture (1-165) | 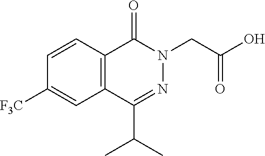 [SMILES: CC(C)c1nn(CC(=O)O)c(=O)c2ccc(C(F)(F)F)cc12] (I-52) | 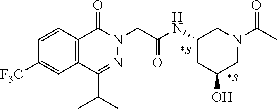 [SMILES: CC(=O)N1C[C@@H](O)C[C@H](NC(=O)Cn2nc(C(C)C)c3cc(C(F)(F)F)ccc3c2=O)C1] | X159 |
| 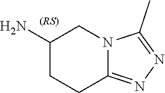 [1251923-84-8] | 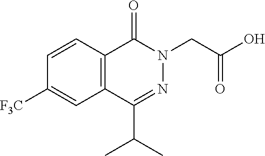 [SMILES: CC(C)c1nn(CC(=O)O)c(=O)c2ccc(C(F)(F)F)cc12] (I-52) | 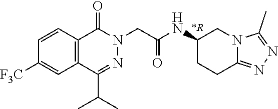 [SMILES: Cc1nnc2n1C[C@H](NC(=O)Cn1nc(C(C)C)c3cc(C(F)(F)F)ccc3c1=O)CC2] | X163 |
| 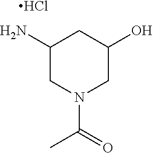 Cis/trans mixture (I-165) | 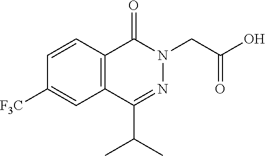 [SMILES: CC(C)c1nn(CC(=O)O)c(=O)c2ccc(C(F)(F)F)cc12] (I-52) | 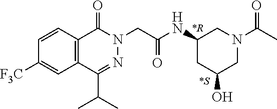 [SMILES: CC(=O)N1C[C@@H](O)C[C@@H](NC(=O)Cn2nc(C(C)C)c3cc(C(F)(F)F)ccc3c2=O)C1] | X164 |
| 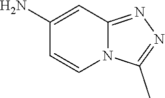 [1214900-87-4] | 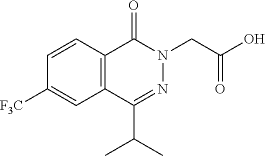 [SMILES: CC(C)c1nn(CC(=O)O)c(=O)c2ccc(C(F)(F)F)cc12] (I-52) | 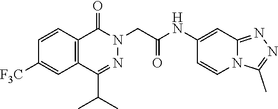 [SMILES: Cc1nnc2cc(NC(=O)Cn3nc(C(C)C)c4cc(C(F)(F)F)ccc4c3=O)ccn12] | X165 |
| 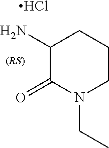 [2126160-37-8] | 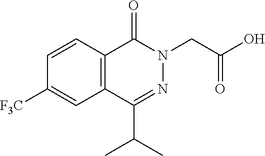 [SMILES: CC(C)c1nn(CC(=O)O)c(=O)c2ccc(C(F)(F)F)cc12] (I-52) | 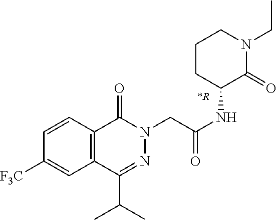 [SMILES: CCN1CCC[C@@H](NC(=O)Cn2nc(C(C)C)c3cc(C(F)(F)F)ccc3c2=O)C1=O] | X166 |
| 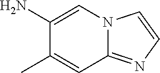 [1215984-92-1] | 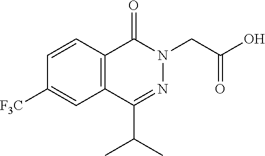 [SMILES: CC(C)c1nn(CC(=O)O)c(=O)c2ccc(C(F)(F)F)cc12] (I-52) | 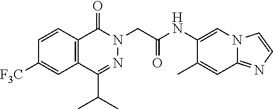 [SMILES: Cc1cc2nccn2cc1NC(=O)Cn1nc(C(C)C)c2cc(C(F)(F)F)ccc2c1=O] | X167 |
| 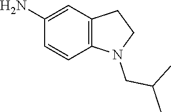 (I-146) | 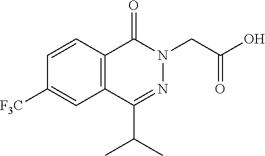 [SMILES: CC(C)c1nn(CC(=O)O)c(=O)c2ccc(C(F)(F)F)cc12] (I-52) | 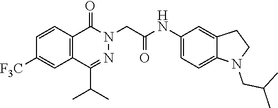 [SMILES: CC(C)CN1CCc2cc(NC(=O)Cn3nc(C(C)C)c4cc(C(F)(F)F)ccc4c3=O)ccc21] | X168 |
| 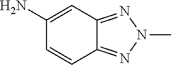 [89852-83-5] | 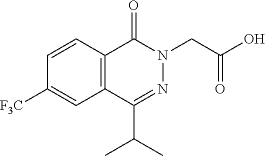 [SMILES: CC(C)c1nn(CC(=O)O)c(=O)c2ccc(C(F)(F)F)cc12] (I-52) | 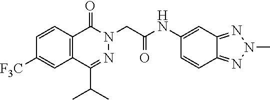 [SMILES: CC(C)c1nn(CC(=O)Nc2ccc3nn(C)nc3c2)c(=O)c2ccc(C(F)(F)F)cc12] | X169 |
| 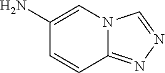 [1087448-58-5] | 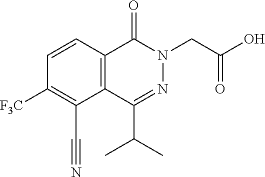 [SMILES: CC(C)c1nn(CC(=O)O)c(=O)c2ccc(C(F)(F)F)c(C#N)c12] (I-73) | 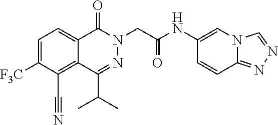 [SMILES: CC(C)c1nn(CC(=O)Nc2ccc3nncn3c2)c(=O)c2ccc(C(F)(F)F)c(C#N)c12] | X170 |
| 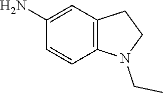 (I-145) | 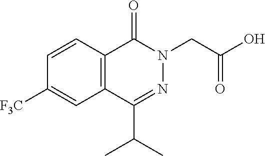 [SMILES: CC(C)c1nn(CC(=O)O)c(=O)c2ccc(C(F)(F)F)cc12] (I-52) | 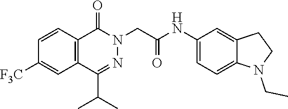 [SMILES: CCN1CCc2cc(NC(=O)Cn3nc(C(C)C)c4cc(C(F)(F)F)ccc4c3=O)ccc21] | X171 |
| 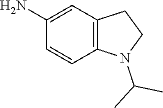 (I-144) | 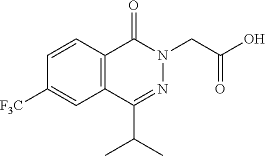 [SMILES: CC(C)c1nn(CC(=O)O)c(=O)c2ccc(C(F)(F)F)cc12] (I-52) | 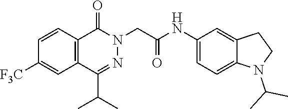 [SMILES: CC(C)c1nn(CC(=O)Nc2ccc3c(c2)CCN3C(C)C)c(=O)c2ccc(C(F)(F)F)cc12] | X173 |
| 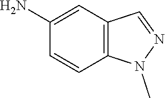 [50593-24-3] | 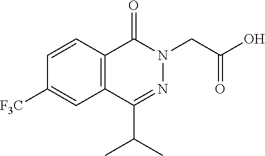 [SMILES: CC(C)c1nn(CC(=O)O)c(=O)c2ccc(C(F)(F)F)cc12] (I-52) | 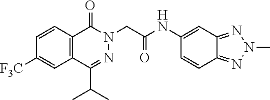 [SMILES: CC(C)c1nn(CC(=O)Nc2ccc3nn(C)nc3c2)c(=O)c2ccc(C(F)(F)F)cc12] | X175 |
| 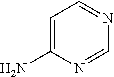 [591-54-8 | 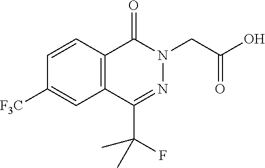 [SMILES: CC(C)(F)c1nn(CC(=O)O)c(=O)c2ccc(C(F)(F)F)cc12] (I-70) | 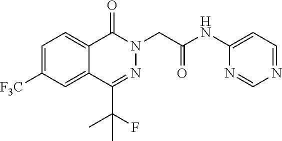 [SMILES: CC(C)(F)c1nn(CC(=O)Nc2ccncn2)c(=O)c2ccc(C(F)(F)F)cc12] | X180 |
| 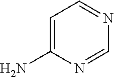 [591-54-8] | 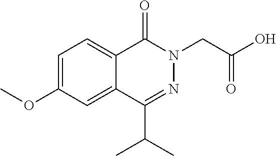 [SMILES: COc1ccc2c(=O)n(CC(=O)O)nc(C(C)C)c2c1] | 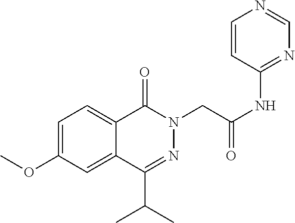 [SMILES: COc1ccc2c(=O)n(CC(=O)Nc3ccncn3)nc(C(C)C)c2c1] | X-1025 |
| 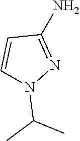 I-1087 | 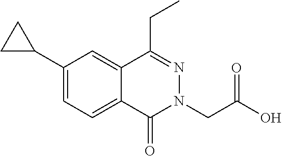 [SMILES: CCc1nn(CC(=O)O)c(=O)c2ccc(C3CC3)cc12] | 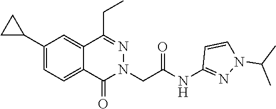 [SMILES: CCc1nn(CC(=O)Nc2ccn(C(C)C)n2)c(=O)c2ccc(C3CC3)cc12] | X-1026 |
| 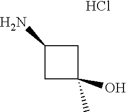 [1523606-23-6] | 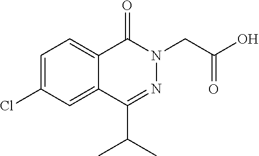 [SMILES: CC(C)c1nn(CC(=O)O)c(=O)c2ccc(Cl)cc12] I-1104 | 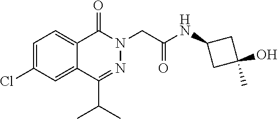 [SMILES: CC(C)c1nn(CC(=O)N[C@H]2C[C@@](C)(O)C2)c(=O)c2ccc(Cl)cc12] | X-1027 |
| 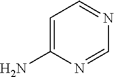 [591-54-8] | 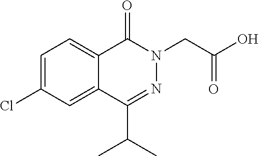 [SMILES: CC(C)c1nn(CC(=O)O)c(=O)c2ccc(Cl)cc12] I-1104 | 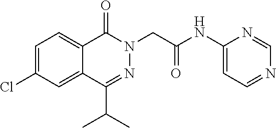 [SMILES: CC(C)c1nn(CC(=O)Nc2ccncn2)c(=O)c2ccc(Cl)cc12] | X-1028 |
| 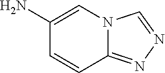 [1082448-58-5] | 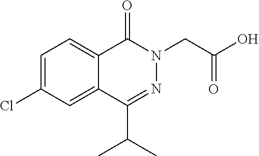 [SMILES: CC(C)c1nn(CC(=O)O)c(=O)c2ccc(Cl)cc12] I-1104 | 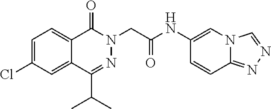 [SMILES: CC(C)c1nn(CC(=O)Nc2ccc3nncn3c2)c(=O)c2ccc(Cl)cc12] | X-1029 |
| 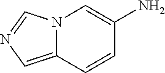 [1508379-00-7] | 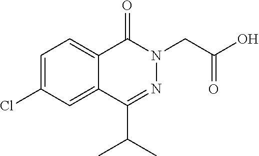 [SMILES: CC(C)c1nn(CC(=O)O)c(=O)c2ccc(Cl)cc12] I-1104 | 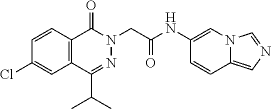 [SMILES: CC(C)c1nn(CC(=O)Nc2ccc3cncn3c2)c(=O)c2ccc(Cl)cc12] | X-1030 |
| 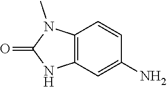 [54732-89-7] | 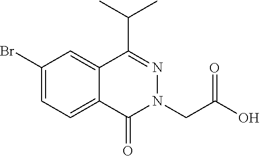 [SMILES: CC(C)c1nn(CC(=O)O)c(=O)c2ccc(Br)cc12] I-1131 | 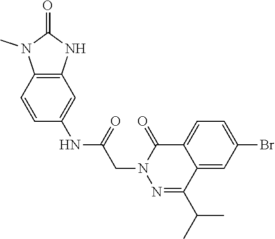 [SMILES: CC(C)c1nn(CC(=O)Nc2ccc3c(c2)[nH]c(=O)n3C)c(=O)c2ccc(Br)cc12] | X-1031 |
| 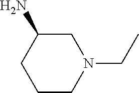 I-1002 | 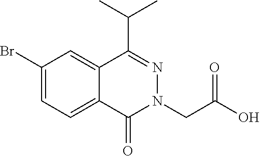 [SMILES: CC(C)c1nn(CC(=O)O)c(=O)c2ccc(Br)cc12] I-1131 | 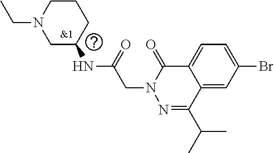 [SMILES: CCN1CCC[C@@H](NC(=O)Cn2nc(C(C)C)c3cc(Br)ccc3c2=O)C1] | X-1032 |
| 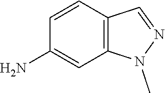 [74728-65-7] | 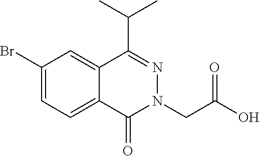 [SMILES: CC(C)c1nn(CC(=O)O)c(=O)c2ccc(Br)cc12] I-1131 | 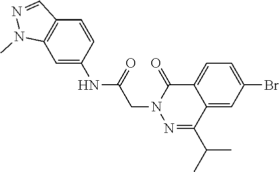 [SMILES: CC(C)c1nn(CC(=O)Nc2ccc3cnn(C)c3c2)c(=O)c2ccc(Br)cc12] | X-1033 |
| 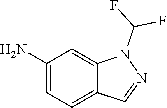 [SMILES: Nc1ccc2cnn(C(F)F)c2c1] I-156 | 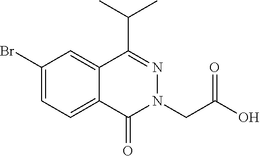 [SMILES: CC(C)c1nn(CC(=O)O)c(=O)c2ccc(Br)cc12] I-1131 | 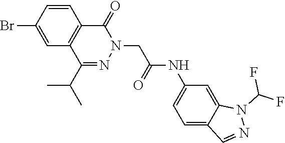 [SMILES: CC(C)c1nn(CC(=O)Nc2ccc3cnn(C(F)F)c3c2)c(=O)c2ccc(Br)cc12] | X-1034 |
| 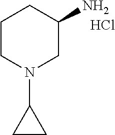 I-200 | 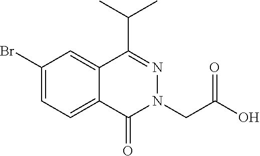 [SMILES: CC(C)c1nn(CC(=O)O)c(=O)c2ccc(Br)cc12] I-1131 | 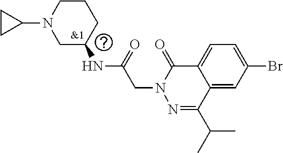 [SMILES: CC(C)c1nn(CC(=O)N[C@@H]2CCCN(C3CC3)C2)c(=O)c2ccc(Br)cc12] | X-1035 |
| 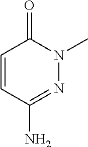 [13506-28-0] | 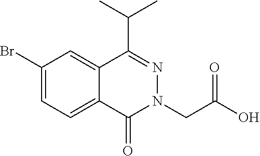 [SMILES: CC(C)c1nn(CC(=O)O)c(=O)c2ccc(Br)cc12] I-1131 | 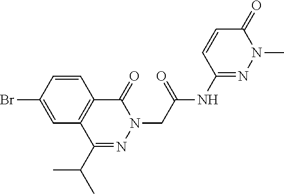 [SMILES: CC(C)c1nn(CC(=O)Nc2ccc(=O)n(C)n2)c(=O)c2ccc(Br)cc12] | X-1036 |
| 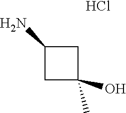 [1523606-23-6] | 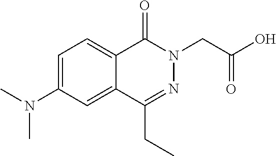 [SMILES: CCc1nn(CC(=O)O)c(=O)c2ccc(N(C)C)cc12] I-1106 | 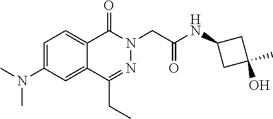 | X-1037 |
| 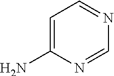 [591-54-8] | 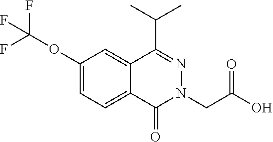 [SMILES: CC(C)c1nn(CC(=O)O)c(=O)c2ccc(OC(F)(F)F)cc12] I-1107 | 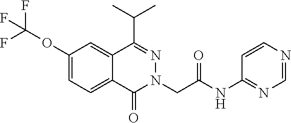 [SMILES: CC(C)c1nn(CC(=O)Nc2ccncn2)c(=O)c2ccc(OC(F)(F)F)cc12] | X-1038 |
| 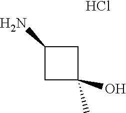 [1523606-23-6] | 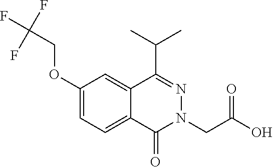 [SMILES: CC(C)c1nn(CC(=O)O)c(=O)c2ccc(OCC(F)(F)F)cc12] I-1116 | 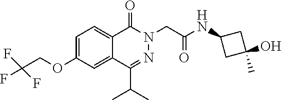 [SMILES: CC(C)c1nn(CC(=O)N[C@H]2C[C@@](C)(O)C2)c(=O)c2ccc(OCC(F)(F)F)cc12] | X-1039 |
| 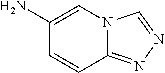 [1082448-58-5] | 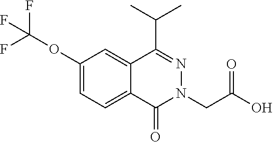 [SMILES: CC(C)c1nn(CC(=O)O)c(=O)c2ccc(OC(F)(F)F)cc12] I-1107 | 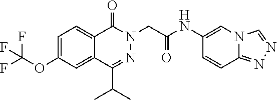 [SMILES: CC(C)c1nn(CC(=O)Nc2ccc3nncn3c2)c(=O)c2ccc(OC(F)(F)F)cc12] | X-1040 |
| 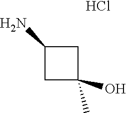 [1523606-23-6] | 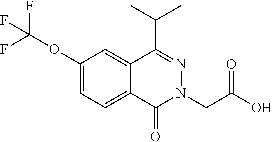 [SMILES: CC(C)c1nn(CC(=O)O)c(=O)c2ccc(OC(F)(F)F)cc12] I-1107 | 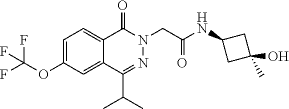 [SMILES: CC(C)c1nn(CC(=O)N[C@H]2C[C@@](C)(O)C2)c(=O)c2ccc(OC(F)(F)F)cc12] | X-1041 |
| 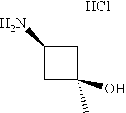 [1523606-23-6] | 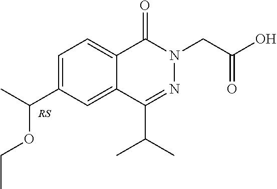 [SMILES: CCOC(C)c1ccc2c(=O)n(CC(=O)O)nc(C(C)C)c2c1] I-1117 | 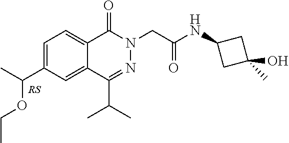 [SMILES: CCOC(C)c1ccc2c(=O)n(CC(=O)N[C@H]3C[C@@](C)(O)C3)nc(C(C)C)c2c1] | X-1042 |
| 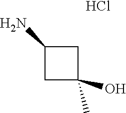 [1523606-23-6] | 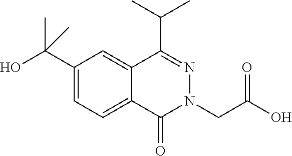 [SMILES: CC(C)c1nn(CC(=O)O)c(=O)c2ccc(C(C)(C)O)cc12] I-1052 | 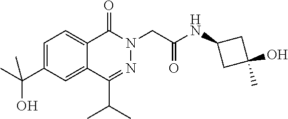 [SMILES: CC(C)c1nn(CC(=O)N[C@H]2C[C@@](C)(O)C2)c(=O)c2ccc(C(C)(C)O)cc12] | X-1043 |
| 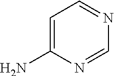 [591-54-8] | 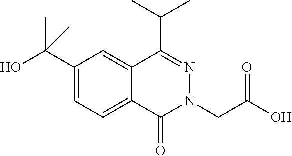 [SMILES: CC(C)c1nn(CC(=O)O)c(=O)c2ccc(C(C)(C)O)cc12] I-1052 | 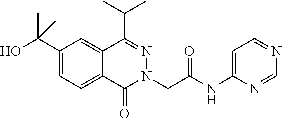 [SMILES: CC(C)c1nn(CC(=O)Nc2ccncn2)c(=O)c2ccc(C(C)(C)O)cc12] | X-1044 |
| 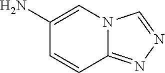 [1082448-58-5] | 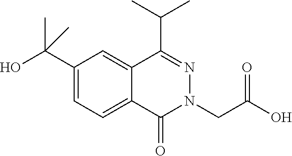 [SMILES: CC(C)c1nn(CC(=O)O)c(=O)c2ccc(C(C)(C)O)cc12] I-1052 | 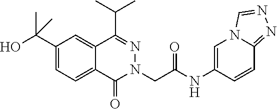 [SMILES: CC(C)c1nn(CC(=O)Nc2ccc3nncn3c2)c(=O)c2ccc(C(C)(C)O)cc12] | X-1045 |
| 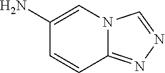 [1082448-58-5] | 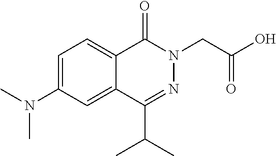 [SMILES: CC(C)c1nn(CC(=O)O)c(=O)c2ccc(N(C)C)cc12] I-1105 | 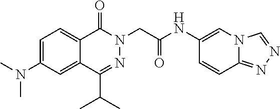 [SMILES: CC(C)c1nn(CC(=O)Nc2ccc3nncn3c2)c(=O)c2ccc(N(C)C)cc12] | X-1046 |
| 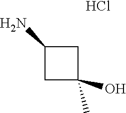 [1523606-23-6] | 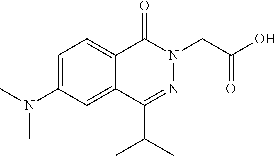 [SMILES: CC(C)c1nn(CC(=O)O)c(=O)c2ccc(N(C)C)cc12] I-1105 | 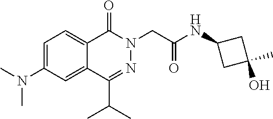 | X-1047 |
| 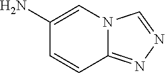 [1082448-58-5] | 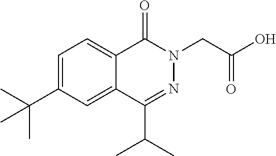 [SMILES: CC(C)c1nn(CC(=O)O)c(=O)c2ccc(C(C)(C)C)cc12] I-1108 | 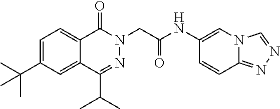 [SMILES: CC(C)c1nn(CC(=O)Nc2ccc3nncn3c2)c(=O)c2ccc(C(C)(C)C)cc12] | X-1048 |
| 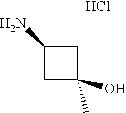 [1523606-23-6] | 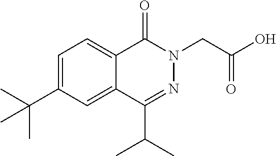 [SMILES: CC(C)c1nn(CC(=O)O)c(=O)c2ccc(C(C)(C)C)cc12] I-1108 | 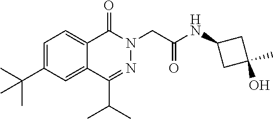 [SMILES: CC(C)c1nn(CC(=O)N[C@H]2C[C@@](C)(O)C2)c(=O)c2ccc(C(C)(C)C)cc12] | X-1049 |
| 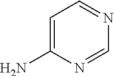 [591-54-8] | 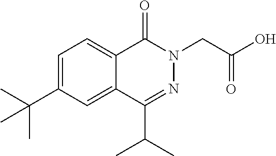 [SMILES: CC(C)c1nn(CC(=O)O)c(=O)c2ccc(C(C)(C)C)cc12] I-1108 | 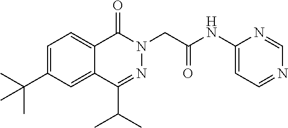 [SMILES: CC(C)c1nn(CC(=O)Nc2ccncn2)c(=O)c2ccc(C(C)(C)C)cc12] | X-1050 |
| 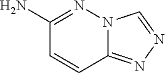 | 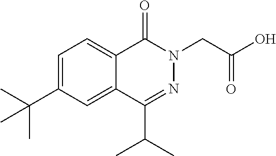 [SMILES: CC(C)c1nn(CC(=O)O)c(=O)c2ccc(C(C)(C)C)cc12] I-1108 | 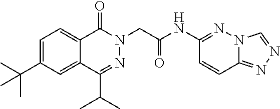 [SMILES: CC(C)c1nn(CC(=O)Nc2ccc3nncn3n2)c(=O)c2ccc(C(C)(C)C)cc12] | X-1051 |
| 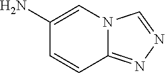 [1082448-58-5] | 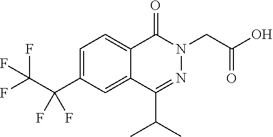 [SMILES: CC(C)c1nn(CC(=O)O)c(=O)c2ccc(C(F)(F)C(F)(F)F)cc12] I-1109 | 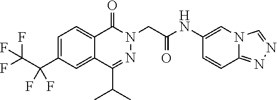 [SMILES: CC(C)c1nn(CC(=O)Nc2ccc3nncn3c2)c(=O)c2ccc(C(F)(F)C(F)(F)F)cc12] | X-1052 |
| 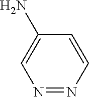 [20744-39-2 | 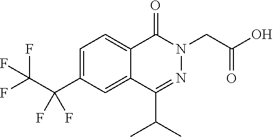 [SMILES: CC(C)c1nn(CC(=O)O)c(=O)c2ccc(C(F)(F)C(F)(F)F)cc12] I-1109 | 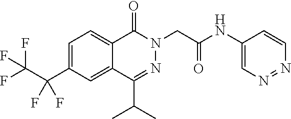 [SMILES: CC(C)c1nn(CC(=O)Nc2ccnnc2)c(=O)c2ccc(C(F)(F)C(F)(F)F)cc12] | X-1053 |
| 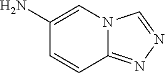 [1082448-58-5] | 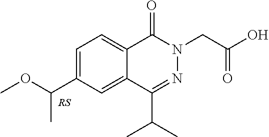 [SMILES: COC(C)c1ccc2c(=O)n(CC(=O)O)nc(C(C)C)c2c1] I-1118 | 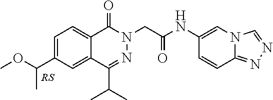 [SMILES: COC(C)c1ccc2c(=O)n(CC(=O)Nc3ccc4nncn4c3)nc(C(C)C)c2c1] | X-1054 |
| 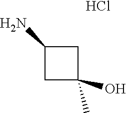 [1523606-23-6] | 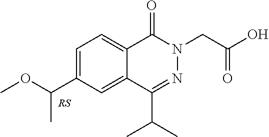 [SMILES: COC(C)c1ccc2c(=O)n(CC(=O)O)nc(C(C)C)c2c1] I-1118 | 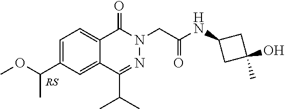 [SMILES: COC(C)c1ccc2c(=O)n(CC(=O)N[C@H]3C[C@@](C)(O)C3)nc(C(C)C)c2c1] | X-1055 |
| 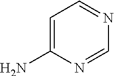 [591-54-8] | 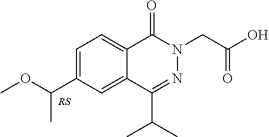 [SMILES: COC(C)c1ccc2c(=O)n(CC(=O)O)nc(C(C)C)c2c1] I-1118 | 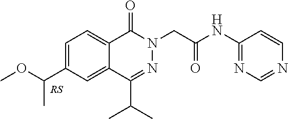 [SMILES: COC(C)c1ccc2c(=O)n(CC(=O)Nc3ccncn3)nc(C(C)C)c2c1] | X-1056 |
| 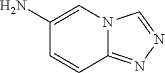 [1082448-58-5] | 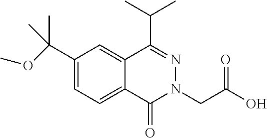 [SMILES: COC(C)(C)c1ccc2c(=O)n(CC(=O)O)nc(C(C)C)c2c1] I-1119 | 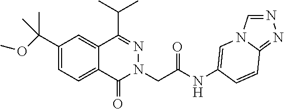 [SMILES: COC(C)(C)c1ccc2c(=O)n(CC(=O)Nc3ccc4nncn4c3)nc(C(C)C)c2c1] | X-1057 |
| 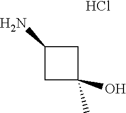 [1523606-23-6] | 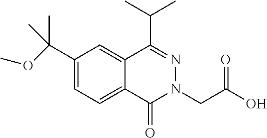 [SMILES: COC(C)(C)c1ccc2c(=O)n(CC(=O)O)nc(C(C)C)c2c1] I-1119 | 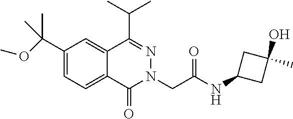 | X-1058 |
| 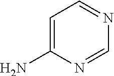 [591-54-8] | 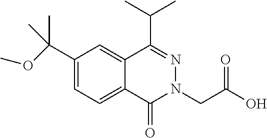 [SMILES: COC(C)(C)c1ccc2c(=O)n(CC(=O)O)nc(C(C)C)c2c1] I-1119 | 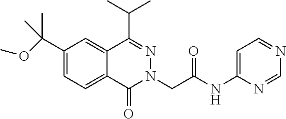 [SMILES: COC(C)(C)c1ccc2c(=O)n(CC(=O)Nc3ccncn3)nc(C(C)C)c2c1] | X-1059 |
| 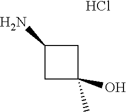 [1523606-23-6] | I-1110 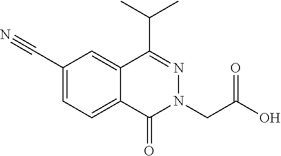 [SMILES: CC(C)c1nn(CC(=O)O)c(=O)c2ccc(C#N)cc12] | 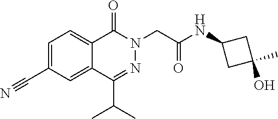 [SMILES: CC(C)c1nn(CC(=O)N[C@H]2C[C@@](C)(O)C2)c(=O)c2ccc(C#N)cc12] | X-1060 |
| 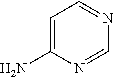 [591-54-8] | I-1110 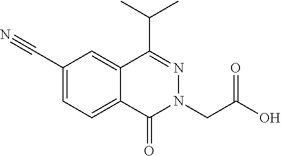 [SMILES: CC(C)c1nn(CC(=O)O)c(=O)c2ccc(C#N)cc12] | 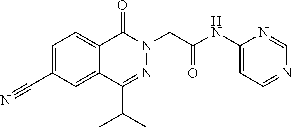 [SMILES: CC(C)c1nn(CC(=O)Nc2ccncn2)c(=O)c2ccc(C#N)cc12] | X-1061 |
| 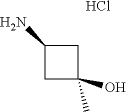 [1523606-23-6] | I-1111 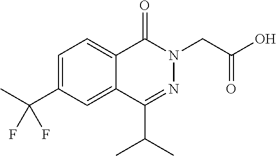 [SMILES: CC(C)c1nn(CC(=O)O)c(=O)c2ccc(C(C)(F)F)cc12] | 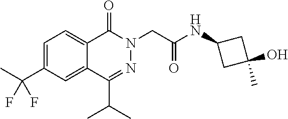 [SMILES: CC(C)c1nn(CC(=O)N[C@H]2C[C@@](C)(O)C2)c(=O)c2ccc(C(C)(F)F)cc12] | X-1062 |
| 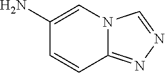 [1082448-58-5] | I-1111 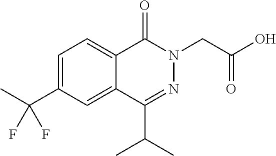 [SMILES: CC(C)c1nn(CC(=O)O)c(=O)c2ccc(C(C)(F)F)cc12] | 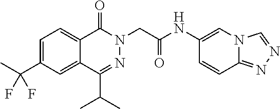 [SMILES: CC(C)c1nn(CC(=O)Nc2ccc3nncn3c2)c(=O)c2ccc(C(C)(F)F)cc12] | X-1063 |
| 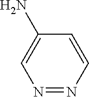 [20744-39-2 | I-1111 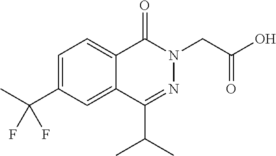 [SMILES: CC(C)c1nn(CC(=O)O)c(=O)c2ccc(C(C)(F)F)cc12] | 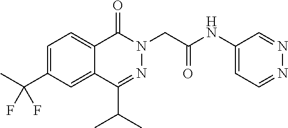 [SMILES: CC(C)c1nn(CC(=O)Nc2ccnnc2)c(=O)c2ccc(C(C)(F)F)cc12] | X-1064 |
| 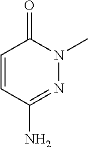 [13506-28-0] | I-1111 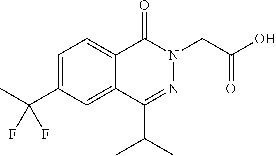 [SMILES: CC(C)c1nn(CC(=O)O)c(=O)c2ccc(C(C)(F)F)cc12] | 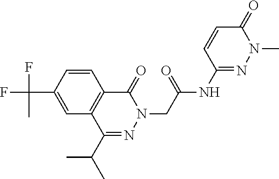 [SMILES: CC(C)c1nn(CC(=O)Nc2ccc(=O)n(C)n2)c(=O)c2ccc(C(C)(F)F)cc12] | X-1065 |
| 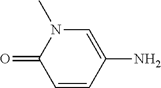 [33630-96-5] | I-1111 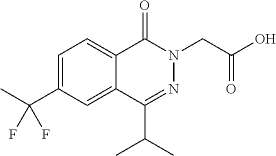 [SMILES: CC(C)c1nn(CC(=O)O)c(=O)c2ccc(C(C)(F)F)cc12] | 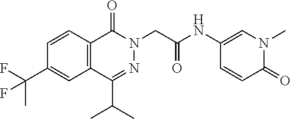 [SMILES: CC(C)c1nn(CC(=O)Nc2ccc(=O)n(C)c2)c(=O)c2ccc(C(C)(F)F)cc12] | X-1066 |
| 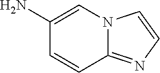 [235106-53-3] | I-1111 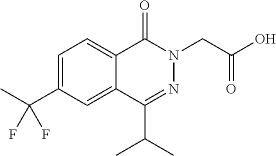 [SMILES: CC(C)c1nn(CC(=O)O)c(=O)c2ccc(C(C)(F)F)cc12] | 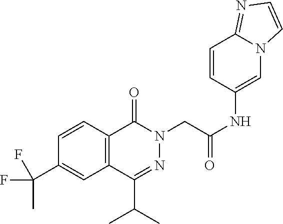 [SMILES: CC(C)c1nn(CC(=O)Nc2ccc3nccn3c2)c(=O)c2ccc(C(C)(F)F)cc12] | X-1067 |
| 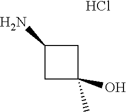 [1523606-23-6] | 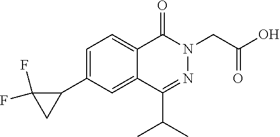 [SMILES: CC(C)c1nn(CC(=O)O)c(=O)c2ccc(C3CC3(F)F)cc12] I-1112 | 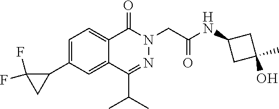 [SMILES: CC(C)c1nn(CC(=O)N[C@H]2C[C@@](C)(O)C2)c(=O)c2ccc(C3CC3(F)F)cc12] | X-1068 |
| 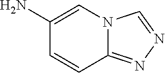 [1082448-58-5] | 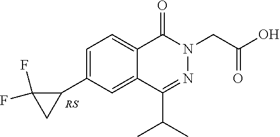 [SMILES: CC(C)c1nn(CC(=O)O)c(=O)c2ccc(C3CC3(F)F)cc12] I-1112 | 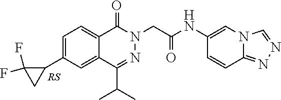 [SMILES: CC(C)c1nn(CC(=O)Nc2ccc3nncn3c2)c(=O)c2ccc(C3CC3(F)F)cc12] | X-1069 |
| 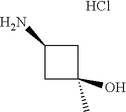 [1523606-23-6] | 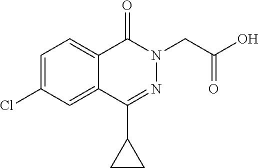 [SMILES: O=C(O)Cn1nc(C2CC2)c2cc(Cl)ccc2c1=O] I-1115 | 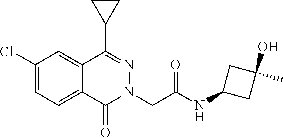 [SMILES: C[C@]1(O)C[C@@H](NC(=O)Cn2nc(C3CC3)c3cc(Cl)ccc3c2=O)C1] | X-1070 |
| 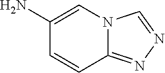 [1082448-58-5] | 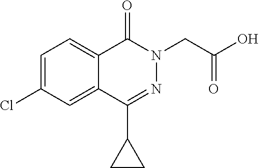 [SMILES: O=C(O)Cn1nc(C2CC2)c2cc(Cl)ccc2c1=O] I-1115 | 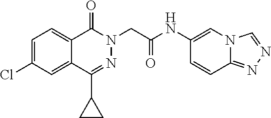 [SMILES: O=C(Cn1nc(C2CC2)c2cc(Cl)ccc2c1=O)Nc1ccc2nncn2c1] | X-1071 |
| 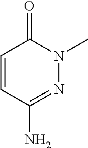 [13506-28-0] | 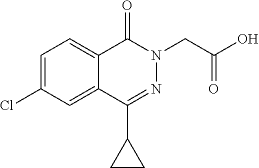 [SMILES: O=C(O)Cn1nc(C2CC2)c2cc(Cl)ccc2c1=O] I-1115 | 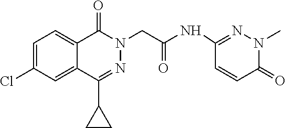 [SMILES: Cn1nc(NC(=O)Cn2nc(C3CC3)c3cc(Cl)ccc3c2=O)ccc1=O] | X-1072 |
| 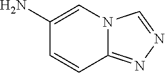 [1082448-58-5] | 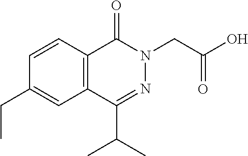 [SMILES: CCc1ccc2c(=O)n(CC(=O)O)nc(C(C)C)c2c1] I-1100 | 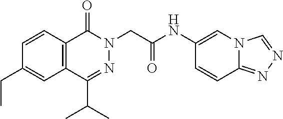 [SMILES: CCc1ccc2c(=O)n(CC(=O)Nc3ccc4nncn4c3)nc(C(C)C)c2c1] | X-1073 |
| 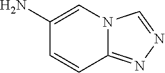 [1082448-58-5] | I-1120 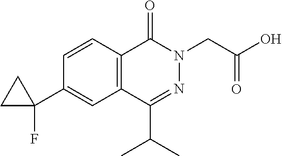 [SMILES: CC(C)c1nn(CC(=O)O)c(=O)c2ccc(C3(F)CC3)cc12] | 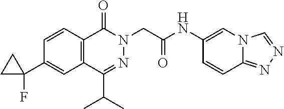 [SMILES: CC(C)c1nn(CC(=O)Nc2ccc3nncn3c2)c(=O)c2ccc(C3(F)CC3)cc12] | X-1074 |
| 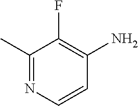 [15931-21-2] | I-1120 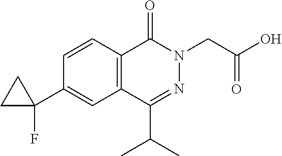 [SMILES: CC(C)c1nn(CC(=O)O)c(=O)c2ccc(C3(F)CC3)cc12] | 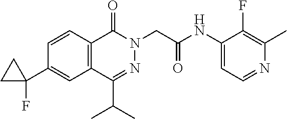 [SMILES: Cc1nccc(NC(=O)Cn2nc(C(C)C)c3cc(C4(F)CC4)ccc3c2=O)c1F] | X-1075 |
| 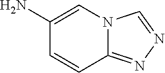 [1082448-58-5] | 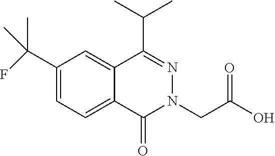 [SMILES: CC(C)c1nn(CC(=O)O)c(=O)c2ccc(C(C)(C)F)cc12] I-1113 | 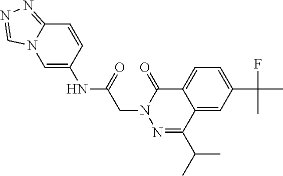 [SMILES: CC(C)c1nn(CC(=O)Nc2ccc3nncn3c2)c(=O)c2ccc(C(C)(C)F)cc12] | X-1076 |
| 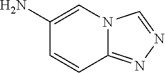 [235106-53-3] | 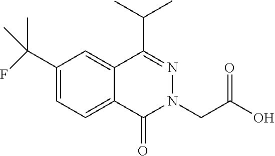 [SMILES: CC(C)c1nn(CC(=O)O)c(=O)c2ccc(C(C)(C)F)cc12] I-1113 | 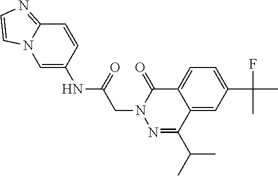 [SMILES: CC(C)c1nn(CC(=O)Nc2ccc3nccn3c2)c(=O)c2ccc(C(C)(C)F)cc12] | X-1077 |
| 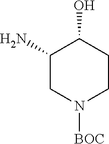 [SMILES: CC(C)(C)OC(=O)N1CC[C@@H](O)[C@@H](N)C1] [1820579-78-9] | 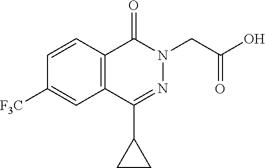 [SMILES: O=C(O)Cn1nc(C2CC2)c2cc(C(F)(F)F)ccc2c1=O] (I-59) | 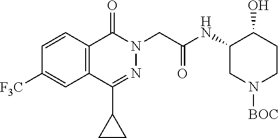 [SMILES: CC(C)(C)OC(=O)N1CC[C@@H](O)[C@@H](NC(=O)Cn2nc(C3CC3)c3cc(C(F)(F)F)ccc3c2=O)C1] | (I-142) |
| 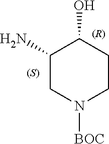 [1638759-83-7] | 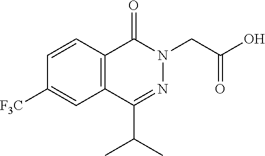 [SMILES: CC(C)c1nn(CC(=O)O)c(=O)c2ccc(C(F)(F)F)cc12] (I-52) | 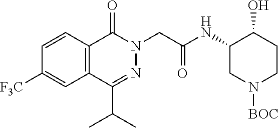 [SMILES: CC(C)c1nn(CC(=O)N[C@H]2CN(C(=O)OC(C)(C)C)CC[C@H]2O)c(=O)c2ccc(C(F)(F)F)cc12] | (I-143) |
| 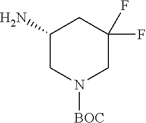 [SMILES: CC(C)(C)OC(=O)N1C[C@H](N)CC(F)(F)C1] [1392473-32-3] | 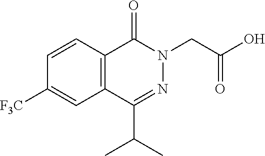 [SMILES: CC(C)c1nn(CC(=O)O)c(=O)c2ccc(C(F)(F)F)cc12] (I-52) | 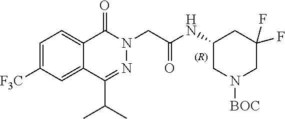 | (I-150) |
| 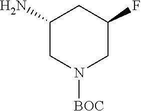 [SMILES: CC(C)(C)OC(=O)N1C[C@H](N)C[C@@H](F)C1] (RS)-trans [1638772-27-6] | 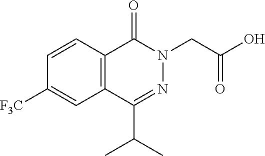 [SMILES: CC(C)c1nn(CC(=O)O)c(=O)c2ccc(C(F)(F)F)cc12] (I-52) | 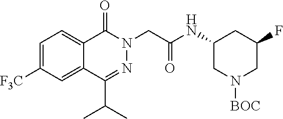 [SMILES: CC(C)c1nn(CC(=O)N[C@@H]2C[C@@H](F)CN(C(=O)OC(C)(C)C)C2)c(=O)c2ccc(C(F)(F)F)cc12] (RS)-trans mixture | (I-152) |
| 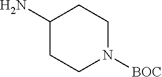 [SMILES: CC(C)(C)OC(=O)N1CCC(N)CC1] [87120-72-7] | 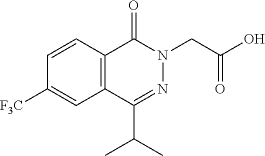 [SMILES: CC(C)c1nn(CC(=O)O)c(=O)c2ccc(C(F)(F)F)cc12] (I-52) | 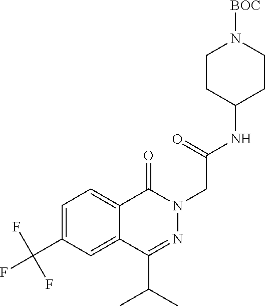 [SMILES: CC(C)c1nn(CC(=O)NC2CCN(C(=O)OC(C)(C)C)CC2)c(=O)c2ccc(C(F)(F)F)cc12] | (I-153) |
| 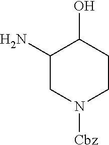 [SMILES: NC1CN(C(=O)OCc2ccccc2)CCC1O] (RS)-cis/trans mixture [203503-30-4] | 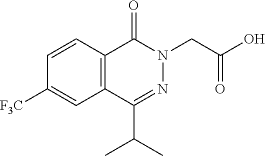 [SMILES: CC(C)c1nn(CC(=O)O)c(=O)c2ccc(C(F)(F)F)cc12] (I-52) | 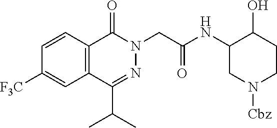 [SMILES: CC(C)c1nn(CC(=O)NC2CN(C(=O)OCc3ccccc3)CCC2O)c(=O)c2ccc(C(F)(F)F)cc12] | (I-151) |
| 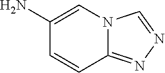 [1082448-58-5] | 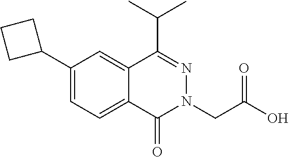 [SMILES: CC(C)c1nn(CC(=O)O)c(=O)c2ccc(C3CCC3)cc12] I-1121 | 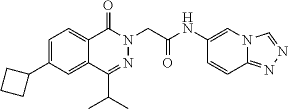 [SMILES: CC(C)c1nn(CC(=O)Nc2ccc3nncn3c2)c(=O)c2ccc(C3CCC3)cc12] | X-1078 |

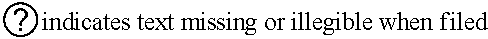

Note: X12 & X43 were isolated by chiral SFC separation of X24; X41 was isolated by chiral SFC separation of X68; X55 and X106 were isolated by chiral SFC separation of X89; X56 and X149 were isolated by chiral SFC separation of X91; X124 and X115 were isolated by chiral SFC separation of X95; X122 and X130 were isolated by chiral SFC separation of X133; X163 was isolated by chiral SFC separation of X68; X166 was isolated by chiral SFC separation of X152

### Example A4

### Synthesis of trans-2-(6-methyl-4-ethyl-1-oxo-phthalazin-2-yl)-N-(4-hydroxycyclohexyl)acetamide (Final Compound 26)

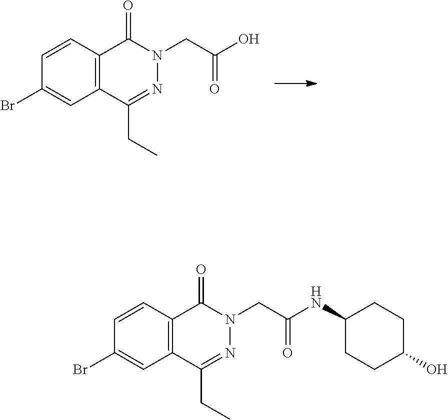 [SMILES: CCc1nn(CC(=O)O)c(=O)c2ccc(Br)cc12 ; CCc1nn(CC(=O)N[C@H]2CC[C@H](O)CC2)c(=O)c2ccc(Br)cc12]

1-Hydroxybenzotriazole [123333-53-9] (84.9 mg, 0.72 mmol) was added to a stirred solution of 2-(6-bromo-4-ethyl-1-oxo-phthalazin-2-yl)acetic acid (I-44) (150 mg, 0.48 mmol) and trans-4-aminocyclohexanol [27489-62-9] (85.5 mg, 0.62 mmol) in anhydrous DCM (5 mL). The mixture was stirred at room temperature for 15 min. Then, 1-(3-dimethylaminopropyl)-3-ethylcarbodiimide hydrochloride [25952-53-8](120 mg, 0.62 mmol) was added and the mixture was stirred at room temperature for 5 h. The mixture was diluted with water and extracted with DCM. The organic layer was separated, dried (MgSO4), filtered and the solvents evaporated in vacuo. The crude product was purified by flash column chromatography (silica, MeOH in DCM 0/100 to 10/90). The desired fractions were collected and concentrated in vacuo to yield trans-2-(6-bromo-4-ethyl-1-oxo-phthalazin-2-yl)-N-(4-hydroxycyclohexyl)acetamide (Final compound 23) (29.2 mg, 15%) as a white solid.

Additional analogs were accessed using similar reaction conditions, using the appropriate reagent.

| Reagent | Intermediate XX | Final compound | Number |
|---|---|---|---|
| 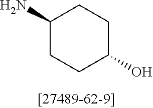 | 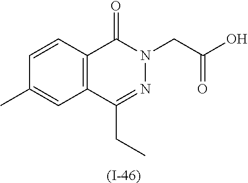 | 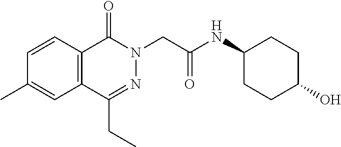 [SMILES: CCc1nn(CC(=O)N[C@H]2CC[C@H](O)CC2)c(=O)c2ccc(C)cc12] | 26 |
| 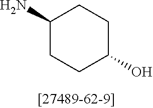 | 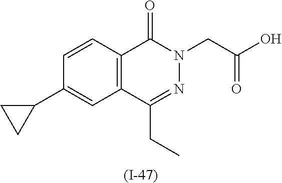 | 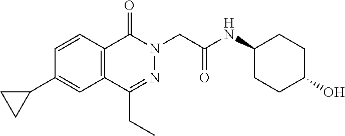 [SMILES: CCc1nn(CC(=O)N[C@H]2CC[C@H](O)CC2)c(=O)c2ccc(C3CC3)cc12] | 27 |
| 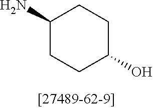 | 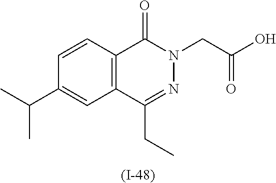 | 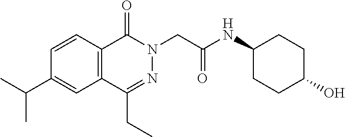 [SMILES: CCc1nn(CC(=O)N[C@H]2CC[C@H](O)CC2)c(=O)c2ccc(C(C)C)cc12] | 28 |
| 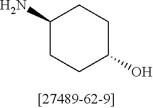 | 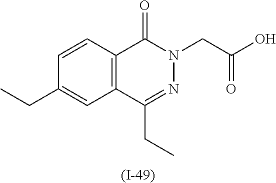 | 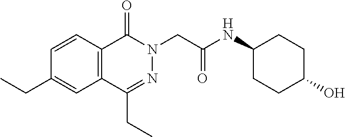 [SMILES: CCc1ccc2c(=O)n(CC(=O)N[C@H]3CC[C@H](O)CC3)nc(CC)c2c1] | 29 |
| 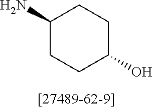 | 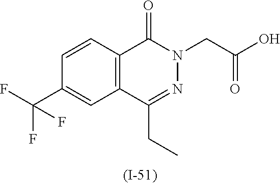 | 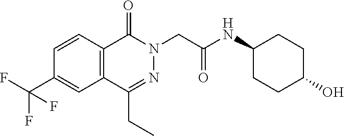 [SMILES: CCc1nn(CC(=O)N[C@H]2CC[C@H](O)CC2)c(=O)c2ccc(C(F)(F)F)cc12] | 30 |

### Example A5

### Synthesis of 2-(6-bromo-4-(1-hydroxyethyl)-1-oxophthalazin-2(1H)-yl)-N-(1-isopropyl-1H-pyrazol-3-yl)acetamide X-1079

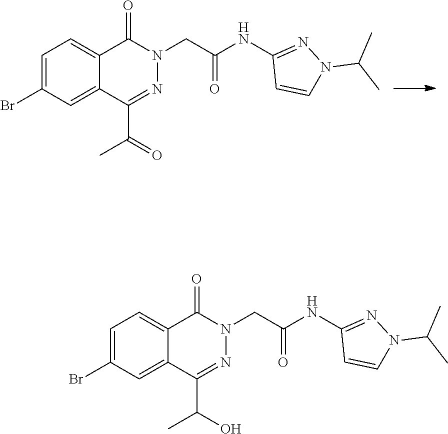 [SMILES: CC(=O)c1nn(CC(=O)Nc2ccn(C(C)C)n2)c(=O)c2ccc(Br)cc12 ; CC(O)c1nn(CC(=O)Nc2ccn(C(C)C)n2)c(=O)c2ccc(Br)cc12]

Sodium borohydride [16940-66-2] (4 mg, 0.1 mmol) was added to a stirred solution of 2-(4-acetyl-6-bromo-1-oxophthalazin-2(1H)-yl)-N-(1-isopropyl-1H-pyrazol-3-yl)acetamide X-1014 (45 mg, 0.1 mmol) in THF (3 mL) and water (1 mL) at 0° C. The resulting mixture was stirred at rt for 30 min NaHCO3 sat. aq. and EtOAc were added and the organic layer was separated, dried over MgSO4 anh, filtered and the solvent was concentrated in vacuo. The crude was purified by flash column chromatography (silica, DCM/MeOH (9:1)/DCM from 0/100 to 100/0). The desired fractions were collected and concentrated in vacuo. The product was triturated with DIPE to yield 2-(6-bromo-4-(1-hydroxyethyl)-1-oxophthalazin-2(1H)-yl)-N-(1-isopropyl-1H-pyrazol-3-yl)acetamide X-1079 (34 mg, 74%) as a white solid.

Structural analogues were synthesized using the same procedure.

| Intermediate | Final compound | Number |
|---|---|---|
| 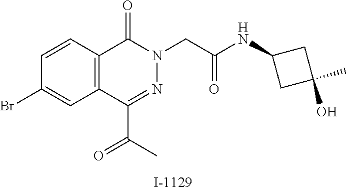 [SMILES: CC(=O)c1nn(CC(=O)N[C@H]2C[C@@](C)(O)C2)c(=O)c2ccc(Br)cc12] | 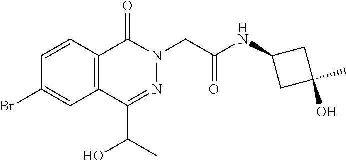 [SMILES: CC(O)c1nn(CC(=O)N[C@H]2C[C@@](C)(O)C2)c(=O)c2ccc(Br)cc12] | X-1080 |
| 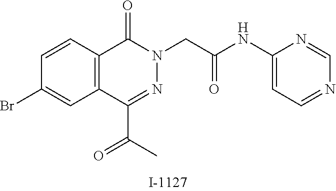 [SMILES: CC(=O)c1nn(CC(=O)Nc2ccncn2)c(=O)c2ccc(Br)cc12] | 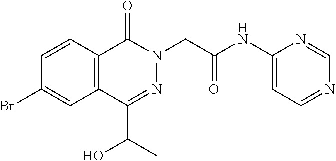 [SMILES: CC(O)c1nn(CC(=O)Nc2ccncn2)c(=O)c2ccc(Br)cc12] | X-1081 |
| 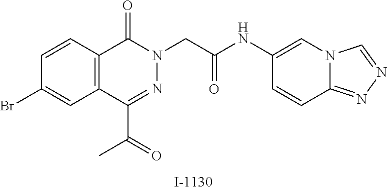 [SMILES: CC(=O)c1nn(CC(=O)Nc2ccc3nncn3c2)c(=O)c2ccc(Br)cc12] | 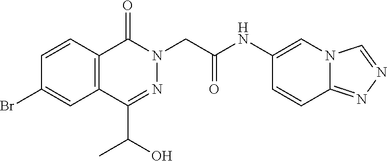 [SMILES: CC(O)c1nn(CC(=O)Nc2ccc3nncn3c2)c(=O)c2ccc(Br)cc12] | X-1082 |
| 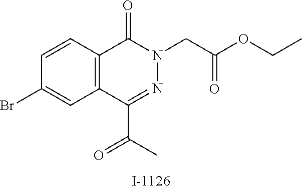 [SMILES: CCOC(=O)Cn1nc(C(C)=O)c2cc(Br)ccc2c1=O] | 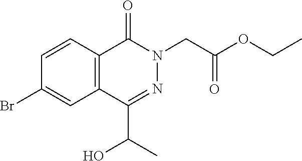 [SMILES: CCOC(=O)Cn1nc(C(C)O)c2cc(Br)ccc2c1=O] | I-1093 |
| 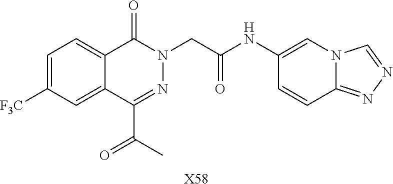 | 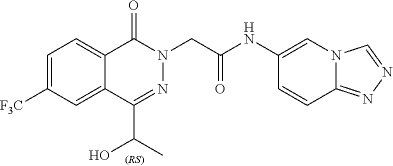 [SMILES: CC(O)c1nn(CC(=O)Nc2ccc3nncn3c2)c(=O)c2ccc(C(F)(F)F)cc12] | X66 |
| 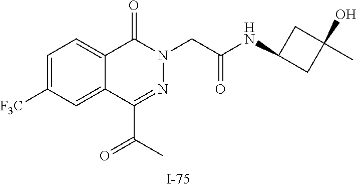 | 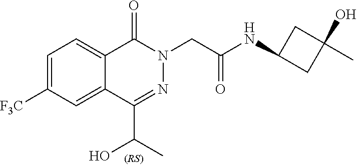 [SMILES: CC(O)c1nn(CC(=O)N[C@H]2C[C@@](C)(O)C2)c(=O)c2ccc(C(F)(F)F)cc12] | X75 |
| 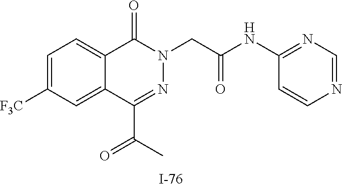 | 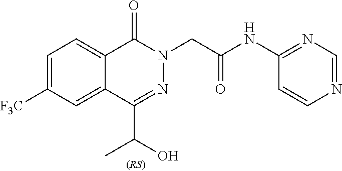 [SMILES: CC(O)c1nn(CC(=O)Nc2ccncn2)c(=O)c2ccc(C(F)(F)F)cc12] | X103 |

### Example A6

### Synthesis of 2-(4-isopropyl-1-oxo-6-(trifluoromethyl)phthalazin-2(1H)-yl)-N-(9H-purin-2-yl)acetamide (Final Compound X1)

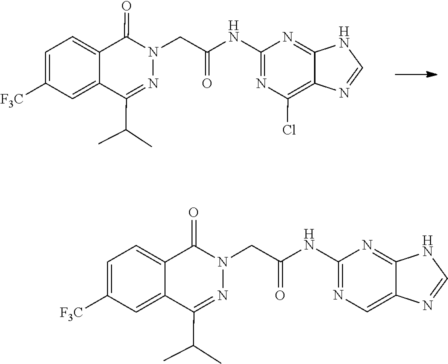 [SMILES: CC(C)c1nn(CC(=O)Nc2nc(Cl)c3nc[nH]c3n2)c(=O)c2ccc(C(F)(F)F)cc12 ; CC(C)c1nn(CC(=O)Nc2ncc3nc[nH]c3n2)c(=O)c2ccc(C(F)(F)F)cc12]

Pd/C (10% wt Pd, 14.8 mg, 0.014 mmol) was added to a stirred solution of N-(6-chloro-9H-purin-2-yl)-2-(4-isopropyl-1-oxo-6-(trifluoromethyl)phthalazin-2(1H)-yl)acetamide X11 (57 mg, 0.12 mmol) and triethylamine (20 μL, 0.15 mmol) in THF (10 mL) at rt. The reaction vessel was filled with hydrogen (3 vacuum/hydrogen cycles) and the mixture stirred under hydrogen atmosphere for 5 hours at rt. The suspension was filtered over Decalite, washing thoroughly with THF and the filtrate was concentrated in vacuo. The crude colorless solid obtained was further purified by preparative HPLC (Stationary phase: RP XBridge Prep C18 OBD-10 μm, 50×250 mm, Mobile phase: 0.25% NH₄HCO₃ solution in water, CH₃CN) followed by preparative SFC (Stationary phase: Chiralpak Daicel IG 20×250 mm, Mobile phase: CO₂, EtOH+0.4 iPrNH₂) to afford X1 as a colorless solid (5 mg, 9%).

### Example A7

### Synthesis of N-((3S,4R)-4-hydroxypiperidin-3-yl)-2-(4-isopropyl-1-oxo-6-(trifluoromethyl)phthalazin-2(1H)-yl)acetamide hydrochloride (I-136)

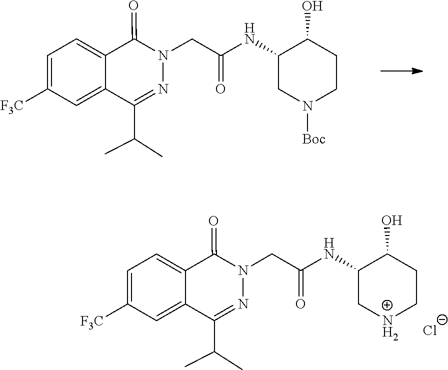 [SMILES: CC(C)c1nn(CC(=O)N[C@H]2CN(C(=O)OC(C)(C)C)CC[C@H]2O)c(=O)c2ccc(C(F)(F)F)cc12 ; CC(C)c1nn(CC(=O)N[C@H]2C[NH2+]CC[C@H]2O)c(=O)c2ccc(C(F)(F)F)cc12 ; [Cl-]]

A 4M solution of HCl in 1,4-dioxane [7647-01-0] (7 mL, 28 mmol) was added to a suspension of tert-butyl (3S,4R)-4-hydroxy-3-(2-(4-isopropyl-1-oxo-6-(trifluoromethyl)phthalazin-2(1H)-yl)acetamido)piperidine-1-carboxylate (I-143) (936 mg, 1.826 mmol) in 1,4-dioxane (6 mL) in a 100-mL RB flask. The reaction mixture was stirred at rt for 1 hour. The solvent was removed to afford crude N-((3S,4R)-4-hydroxypiperidin-3-yl)-2-(4-isopropyl-1-oxo-6-(trifluoromethyl)phthalazin-2(1H)-yl)acetamide (I-136) (880 mg, 98%) as a white powder that was used without further purification.

Additional analogs were accessed using similar reaction conditions, using the appropriate substrate.

| Substrate | Product |
|---|---|
| 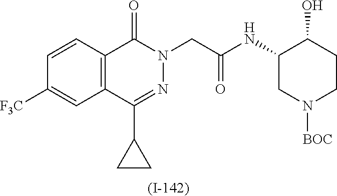 | 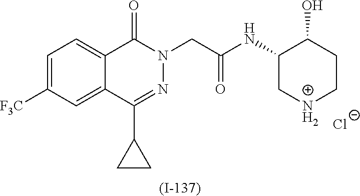 |
| 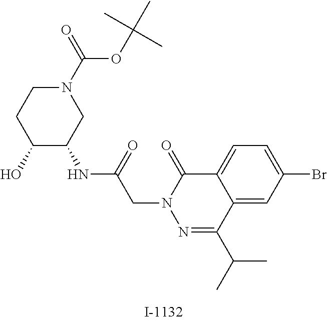 [SMILES: CC(C)c1nn(CC(=O)N[C@H]2CN(C(=O)OC(C)(C)C)CC[C@H]2O)c(=O)c2ccc(Br)cc12] | 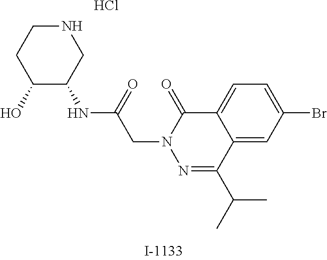 [SMILES: Cl ; CC(C)c1nn(CC(=O)N[C@H]2CNCC[C@H]2O)c(=O)c2ccc(Br)cc12] |
| 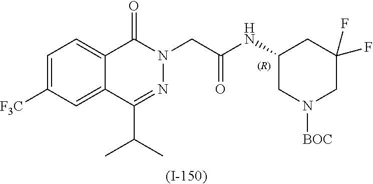 | 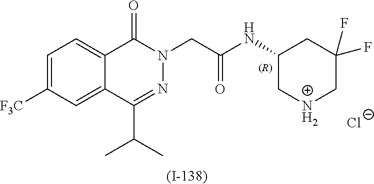 |
| 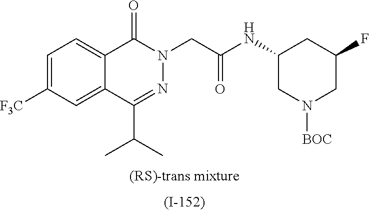 | 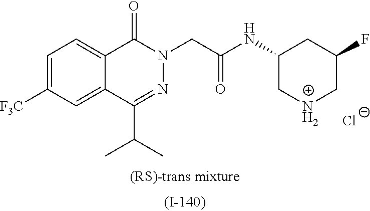 |
| 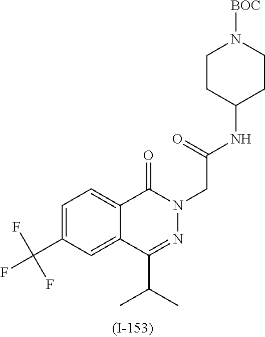 [SMILES: CC(C)c1nn(CC(=O)NC2CCN(C(=O)OC(C)(C)C)CC2)c(=O)c2ccc(C(F)(F)F)cc12] | 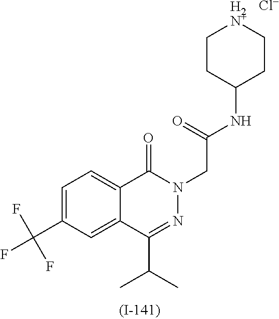 |

### Synthesis of N-(4-hydroxypiperidin-3-yl)-2-(4-isopropyl-1-oxo-6-(trifluoromethyl)phthalazin-2(1H)-yl)acetamide (I-139)

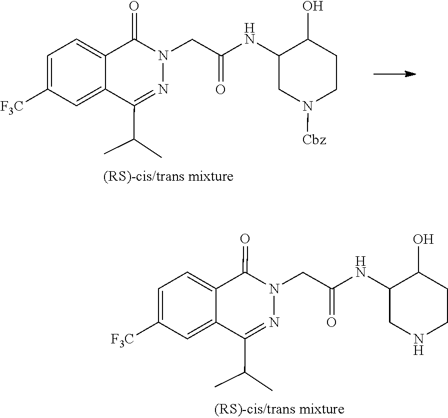 [SMILES: CC(C)c1nn(CC(=O)NC2CN(C(=O)OCc3ccccc3)CCC2O)c(=O)c2ccc(C(F)(F)F)cc12 ; CC(C)c1nn(CC(=O)NC2CNCCC2O)c(=O)c2ccc(C(F)(F)F)cc12]

Pd/C (10% wt. Pd, 75 mg, 0.07 mmol) was added to a solution of I-151 (450 mg, 0.82 mmol) in EtOH (30 mL) under nitrogen. The reaction vessel was placed under hydrogen atmosphere and the reaction mixture stirred at rt for 16 hours. It was filtered over Celite under nitrogen and the filtrate was concentrated under reduced pressure at 40° C. The crude material was dissolved in DCM (50 mL) and filtered on teflon filter and the filtrate was concentrated under reduced pressure at 40° C. to afford I-139 (347 mg, 98%) as a white solid that was used without further purification.

### Synthesis of N-((3S,4R)-1-ethyl-4-hydroxypiperidin-3-yl)-2-(4-isopropyl-1-oxo-6-(trifluoromethyl)phthalazin-2(1H)-yl)acetamide (Final Compound X6)

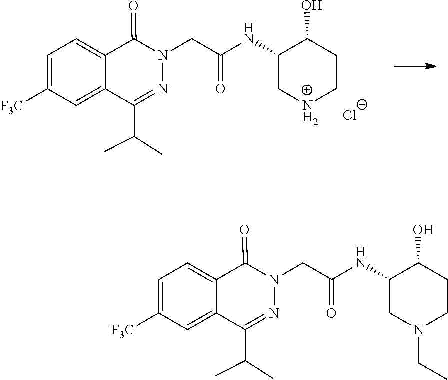 [SMILES: CC(C)c1nn(CC(=O)N[C@H]2C[NH2+]CC[C@H]2O)c(=O)c2ccc(C(F)(F)F)cc12 ; [Cl-] ; CCN1CC[C@@H](O)[C@@H](NC(=O)Cn2nc(C(C)C)c3cc(C(F)(F)F)ccc3c2=O)C1]

Iodoethane [75-03-6] (0.1 mL, 1.244 mmol) was added to a suspension of N-((3S,4R)-4-hydroxypiperidin-3-yl)-2-(4-isopropyl-1-oxo-6-(trifluoromethyl)phthalazin-2(1H)-yl)acetamide hydrochloride (I-136) (203 mg, 0.412 mmol) and triethylamine [121-44-8] (0.6 mL, 4.328 mmol) in dry MeCN (4 mL) in a dry MW vial. The reaction mixture was stirred overnight at rt. The crude mixture was diluted with MeOH (˜18 mL) and purified by preparative HPLC. (Stationary phase: RP XBridge Prep C18 OBD-10 μm, 50×250 mm, Mobile phase: 0.25% NH₄HCO₃ solution in water, MeOH) to afford X6 (149 mg, 82%) as a white powder.

Additional analogs were accessed using similar reaction conditions, using the appropriate substrate.

| Substrate | Final compound | Cpd No |
|---|---|---|
| 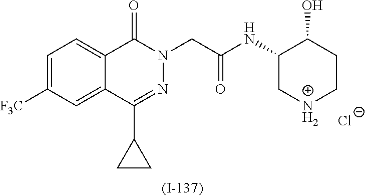 | 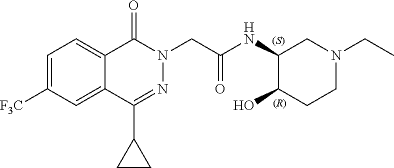 | X19 |
| 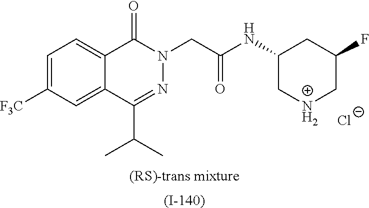 | 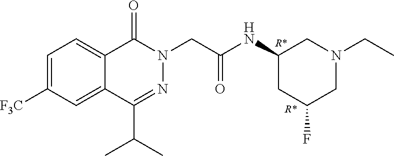 | X37 |
| 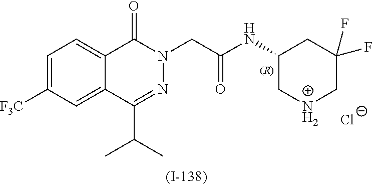 | 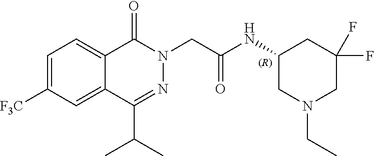 | X117 |
| 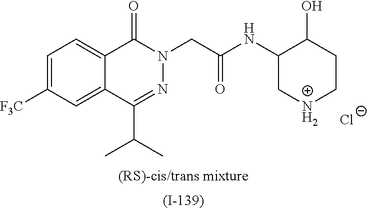 | 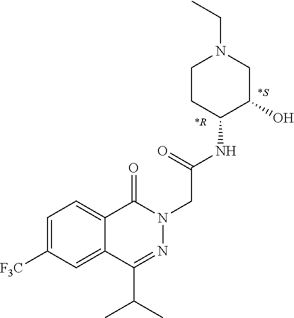 [SMILES: CCN1CC[C@@H](NC(=O)Cn2nc(C(C)C)c3cc(C(F)(F)F)ccc3c2=O)[C@@H](O)C1] | X139 |
| 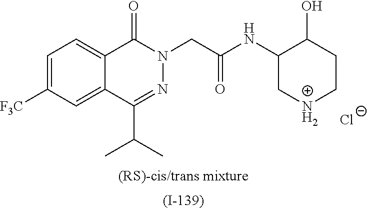 | 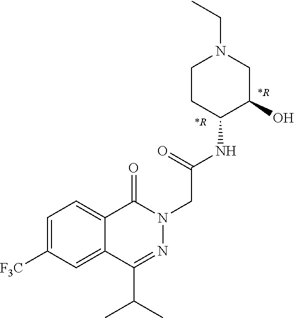 [SMILES: CCN1CC[C@@H](NC(=O)Cn2nc(C(C)C)c3cc(C(F)(F)F)ccc3c2=O)[C@H](O)C1] | X140 |
| 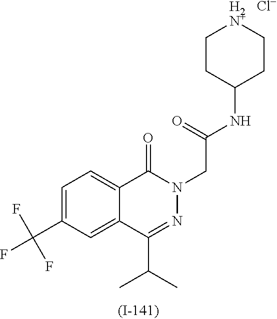 | 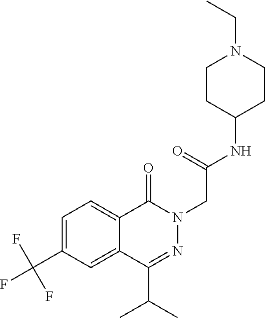 [SMILES: CCN1CCC(NC(=O)Cn2nc(C(C)C)c3cc(C(F)(F)F)ccc3c2=O)CC1] | X143 |
| 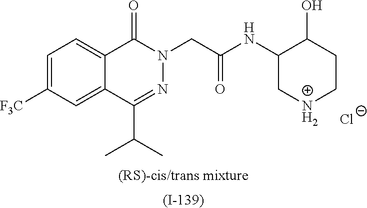 | 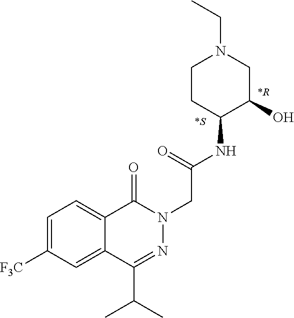 [SMILES: CCN1CC[C@H](NC(=O)Cn2nc(C(C)C)c3cc(C(F)(F)F)ccc3c2=O)[C@H](O)C1] | X147 |
| 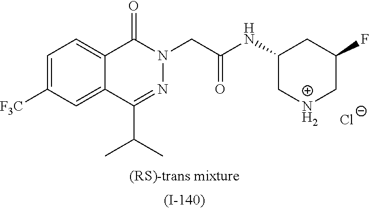 | 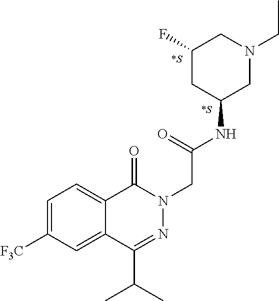 [SMILES: CCN1C[C@@H](F)C[C@H](NC(=O)Cn2nc(C(C)C)c3cc(C(F)(F)F)ccc3c2=O)C1] | X153 |
| 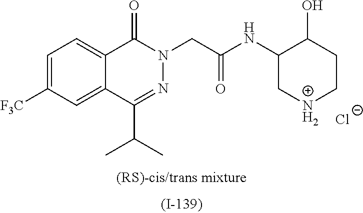 [SMILES: CC(C)c1nn(CC(=O)NC2C[NH2+]CCC2O)c(=O)c2ccc(C(F)(F)F)cc12 ; [Cl-]] | 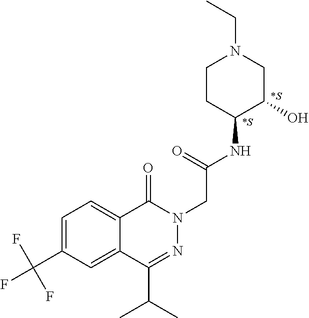 [SMILES: CCN1CC[C@H](NC(=O)Cn2nc(C(C)C)c3cc(C(F)(F)F)ccc3c2=O)[C@@H](O)C1] | X172 |
| 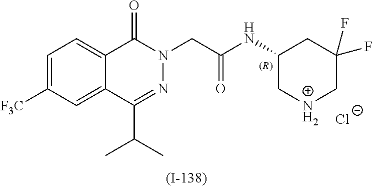 | 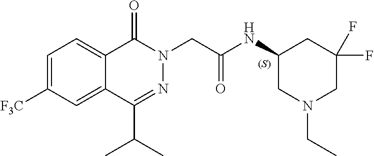 | X174 |
| 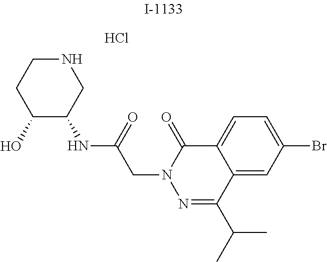 [SMILES: Cl ; CC(C)c1nn(CC(=O)N[C@H]2CNCC[C@H]2O)c(=O)c2ccc(Br)cc12] | 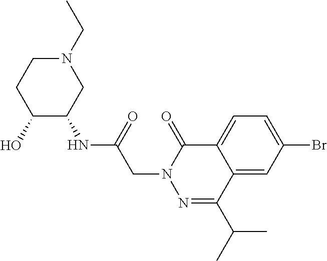 [SMILES: CCN1CC[C@@H](O)[C@@H](NC(=O)Cn2nc(C(C)C)c3cc(Br)ccc3c2=O)C1] | X-1083 |

### Example A8

### Synthesis of N-((3S,4R)-1-cyclopropyl-4-hydroxypiperidin-3-yl)-2-(4-isopropyl-1-oxo-6-(trifluoromethyl)phthalazin-2(1H)-yl)acetamide (Final Compound X15)

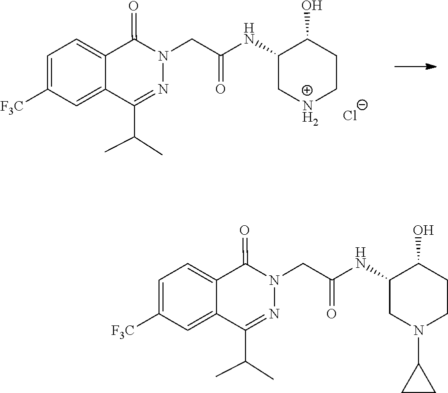 [SMILES: CC(C)c1nn(CC(=O)N[C@H]2C[NH2+]CC[C@H]2O)c(=O)c2ccc(C(F)(F)F)cc12 ; [Cl-] ; CC(C)c1nn(CC(=O)N[C@H]2CN(C3CC3)CC[C@H]2O)c(=O)c2ccc(C(F)(F)F)cc12]

Sodium cyanoborohydride [25895-60-7] (30 mg, 0.477 mmol) was added to a suspension of (I-136) (105 mg, 0.213 mmol), (1-ethoxycyclopropoxy)trimethylsilane [27374-25-0] (45 μL, 0.225 mmol) and acetic acid (0.15 mL, 2.62 mmol) in MeOH (1.5 mL) in a MW vial under nitrogen. The reaction mixture was stirred overnight at 70° C. The crude mixture was purified by preparative HPLC (Stationary phase: RP XBridge Prep C18 OBD-10 μm, 50×250 mm, Mobile phase: 0.25% NH₄HCO₃ solution in water, MeOH) to afford X15 (68 mg, 71%) as a white powder.

Additional analogs were accessed using similar reaction conditions, using the appropriate substrate.

| Substrate | Final compound | Cpd No |
|---|---|---|
| 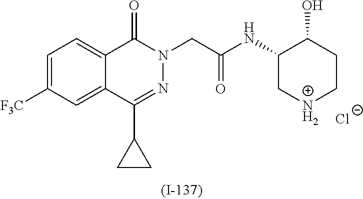 [SMILES: O=C(Cn1nc(C2CC2)c2cc(C(F)(F)F)ccc2c1=O)N[C@H]1C[NH2+]CC[C@H]1O ; [Cl-]] | 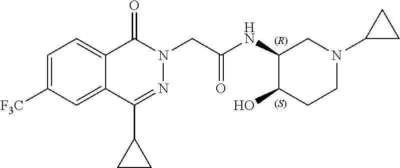 | X45 |
| 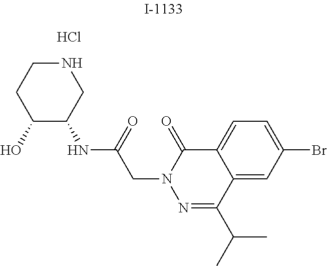 [SMILES: Cl ; CC(C)c1nn(CC(=O)N[C@H]2CNCC[C@H]2O)c(=O)c2ccc(Br)cc12] | 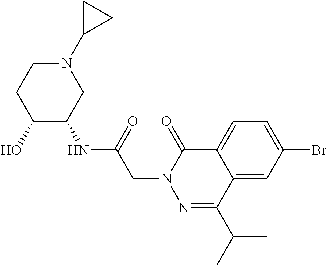 [SMILES: CC(C)c1nn(CC(=O)N[C@H]2CN(C3CC3)CC[C@H]2O)c(=O)c2ccc(Br)cc12] | X-1084 |

### Example A9

### Synthesis of N-((3S,4R)-1-cyclopropyl-4-hydroxy-1-((R*)-3-hydroxybutyl)piperidin-3-yl)-2-(4-isopropyl-1-oxo-6-(trifluoromethyl)phthalazin-2(1H)-yl)acetamide, N-((3S,4R)-4-hydroxy-1-((S*)-3-hydroxybutyl)piperidinhydroxypiperidin-3-yl)-2-(4-isopropyl-1-oxo-6-(trifluoromethyl)phthalazin-2(1H)-yl)acetamide (Final Compounds X70, X81 and X83)

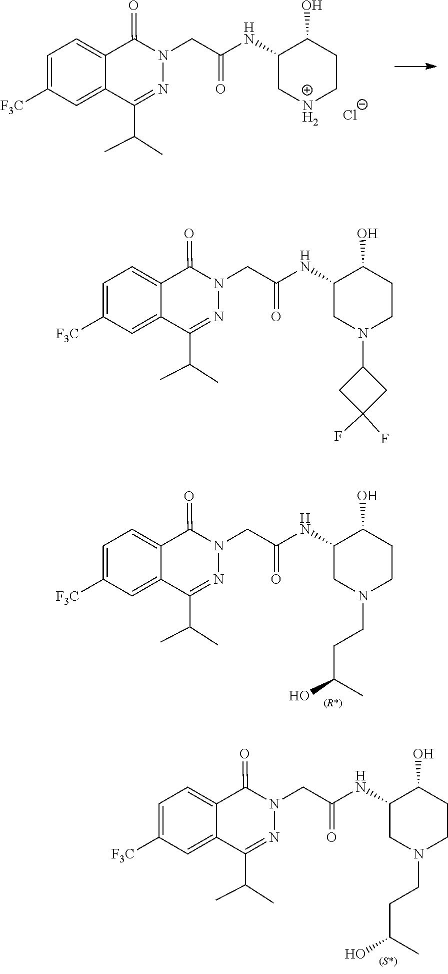 [SMILES: CC(C)c1nn(CC(=O)N[C@H]2C[NH2+]CC[C@H]2O)c(=O)c2ccc(C(F)(F)F)cc12 ; [Cl-] ; CC(C)c1nn(CC(=O)N[C@H]2CN(C3CC(F)(F)C3)CC[C@H]2O)c(=O)c2ccc(C(F)(F)F)cc12 ; CC(C)c1nn(CC(=O)N[C@H]2CN(CC[C@@H](C)O)CC[C@H]2O)c(=O)c2ccc(C(F)(F)F)cc12 ; CC(C)c1nn(CC(=O)N[C@H]2CN(CC[C@H](C)O)CC[C@H]2O)c(=O)c2ccc(C(F)(F)F)cc12]

Sodium cyanoborohydride [25895-60-7] (25 mg, 0.398 mmol) was added to a suspension of (I-136) (95 mg, 0.193 mmol) and 3,3-difluorocyclobutanone [1273564-99-0] (50 mg, 0.471 mmol) in MeOH (1.5 mL) in a MW vial under nitrogen. The reaction mixture was stirred overnight at 40° C. The crude mixture was purified by preparative HPLC (Stationary phase: RP XBridge Prep C18 OBD-10 μm, 50×250 mm, Mobile phase: 0.25% NH₄HCO₃ solution in water, MeOH) to afford X70 (8 mg, 9%) as an off-white solid, X81 (11 mg, 12%) as an off-white solid and X83 (46 mg, 48%) as a colorless solid.

Additional analogs were accessed using similar reaction conditions, using the appropriate reagent.

| Reagent | Intermediate XX | Final compound | Number |
|---|---|---|---|
| 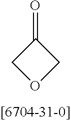 | 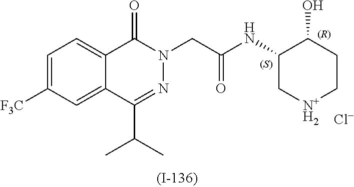 | 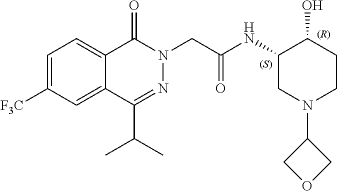 | X104 |

### Example A10

### Synthesis of N-([1,2,4]triazolo[4,3-a]pyrazin-6-yl)-2-(4-isopropyl-1-oxo-6-(trifluoromethyl)phthalazin-2(1H)-yl)acetamide (X14)

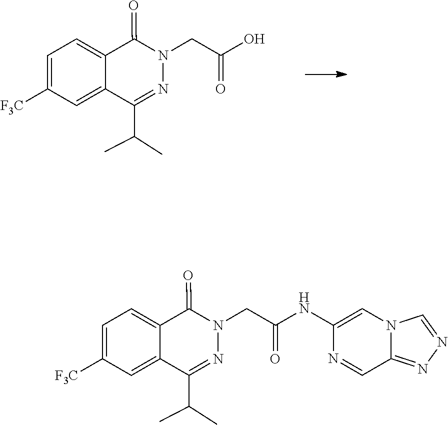 [SMILES: CC(C)c1nn(CC(=O)O)c(=O)c2ccc(C(F)(F)F)cc12 ; CC(C)c1nn(CC(=O)Nc2cn3cnnc3cn2)c(=O)c2ccc(C(F)(F)F)cc12]

TCFH [207915-99-9] (268 mg, 0.95 mmol) was added to a solution of (1-52) (150 mg, 0.48 mmol), [1,2,4]triazolo[4,3-a]pyrazin-6-amine [2111465-25-7] (97 mg, 0.72 mmol) and 1-methylimidazole [616-47-7] (0.19 mL, 1.03 g/mL, 2.39 mmol) in dry MeCN (3.7 mL). The reaction mixture was stirred at rt for 16 hours. The crude mixture was purified by preparative HPLC (Stationary phase: RP XBridge Prep C18 OBD-10 μm, 50×250 mm, Mobile phase: 0.25% NH₄HCO₃ solution in water, CH₃CN) to afford X14 (101 mg, 49%) as a pale tan solid.

Additional analogs were accessed using similar reaction conditions, using the appropriate reagent

| | | | Num- |
|---|---|---|---|
| Reagent | Intermediate XX | Final compound | ber |
| 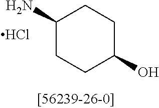 | 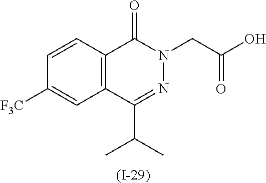 | 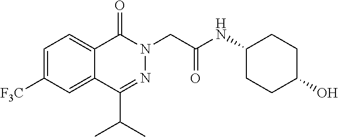 [SMILES: CC(C)c1nn(CC(=O)N[C@H]2CC[C@@H](O)CC2)c(=O)c2ccc(C(F)(F)F)cc12] | X42 |
| 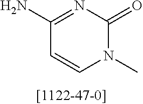 | 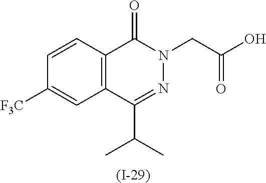 | 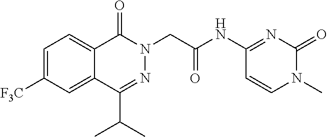 [SMILES: CC(C)c1nn(CC(=O)Nc2ccn(C)c(=O)n2)c(=O)c2ccc(C(F)(F)F)cc12] | X112 |
| 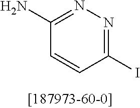 | 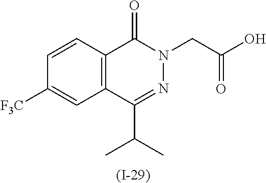 | 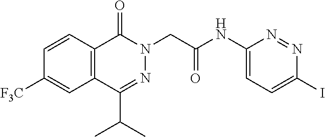 [SMILES: CC(C)c1nn(CC(=O)Nc2ccc(I)nn2)c(=O)c2ccc(C(F)(F)F)cc12] | X154 |
| 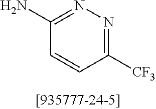 [SMILES: Nc1ccc(C(F)(F)F)nn1] | 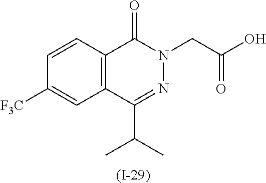 | 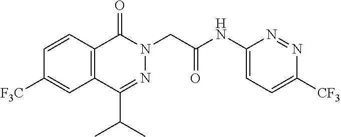 [SMILES: CC(C)c1nn(CC(=O)Nc2ccc(C(F)(F)F)nn2)c(=O)c2ccc(C(F)(F)F)cc12] | X161 |
| 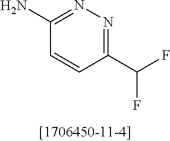 | 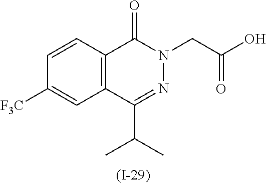 | 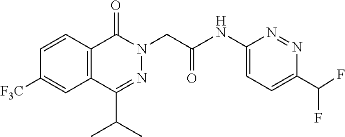 [SMILES: CC(C)c1nn(CC(=O)Nc2ccc(C(F)F)nn2)c(=O)c2ccc(C(F)(F)F)cc12] | X176 |
| 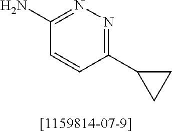 | 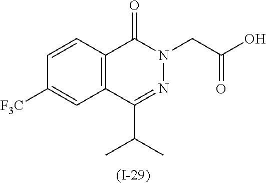 | 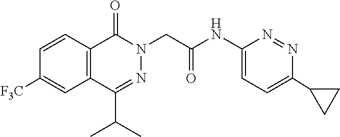 [SMILES: CC(C)c1nn(CC(=O)Nc2ccc(C3CC3)nn2)c(=O)c2ccc(C(F)(F)F)cc12] | X179 |
| 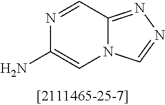 | 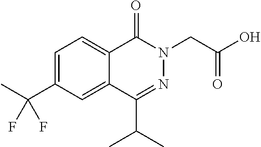 [SMILES: CC(C)c1nn(CC(=O)O)c(=O)c2ccc(C(C)(F)F)cc12] | 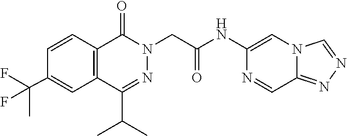 [SMILES: CC(C)c1nn(CC(=O)Nc2cn3cnnc3cn2)c(=O)c2ccc(C(C)(F)F)cc12] | X- 1085 |
| 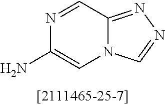 | 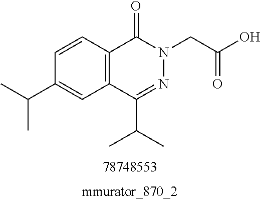 [SMILES: CC(C)c1ccc2c(=O)n(CC(=O)O)nc(C(C)C)c2c1] | 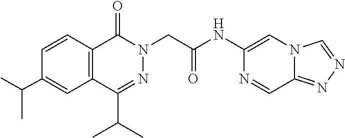 [SMILES: CC(C)c1ccc2c(=O)n(CC(=O)Nc3cn4cnnc4cn3)nc(C(C)C)c2c1] | X- 1086 |
| 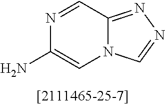 | 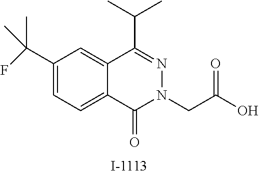 [SMILES: CC(C)c1nn(CC(=O)O)c(=O)c2ccc(C(C)(C)F)cc12] | 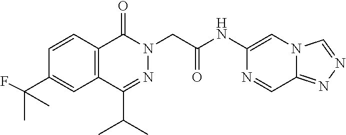 [SMILES: CC(C)c1nn(CC(=O)Nc2cn3cnnc3cn2)c(=O)c2ccc(C(C)(C)F)cc12] | X- 1087 |

### Example A11

### Synthesis of 2-(4-isopropyl-1-oxo-6-(trifluoromethyl)phthalazin-2(1H)-yl)-N-(2-(trifluoromethyl)imidazo[1,2-a]pyridin-6-yl)acetamide (X64)

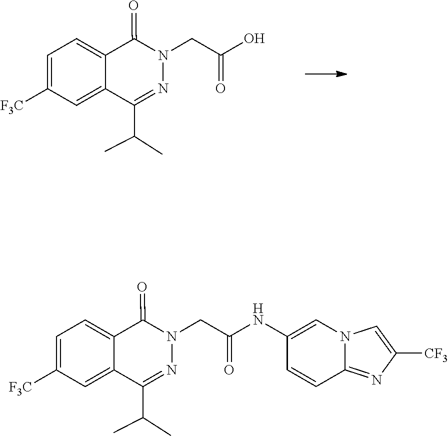 [SMILES: CC(C)c1nn(CC(=O)O)c(=O)c2ccc(C(F)(F)F)cc12 ; CC(C)c1nn(CC(=O)Nc2ccc3nc(C(F)(F)F)cn3c2)c(=O)c2ccc(C(F)(F)F)cc12]

1-chloro-N,N,2-trimethyl-1-propenylamine [26189-59-3] (104 μL, 0.78 mmol) was added to a mixture of carboxylic acid (I-29) (80 mg, 0.25 mmol) in dioxane (2 mL) in a dry vial under nitrogen. The mixture was stirred at rt for 1 hour. 2-(Trifluoromethyl)imidazo[1,2-a]pyridin-6-amine [1343040-93-6] (61.5 mg, 0.31 mmol) was then added, followed by addition of pyridine [110-86-1] (70 μL, 0.98 g/mL, 0.87 mmol). The mixture was stirred at rt for 5 hours. Water was added and the crude product was extracted with EtOAc (3×5 ml) the combined organic layers were dried, filtered and evaporated in vacuo. A purification was performed via preparative HPLC (Stationary phase: RP XBridge Prep C18 OBD-10 μm, 50×250 mm, Mobile phase: 0.25% NH₄HCO₃ solution in water, CH₃CN).to amide X64 (46 mg, 36%) as a dark green solid.

Additional analogs were accessed using similar reaction conditions, using the appropriate reagent.

| | | | Num- |
|---|---|---|---|
| Amine | Substrate | Final compound | ber |
| 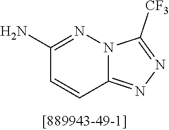 [SMILES: Nc1ccc2nnc(C(F)(F)F)n2n1] | 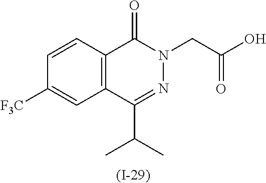 | 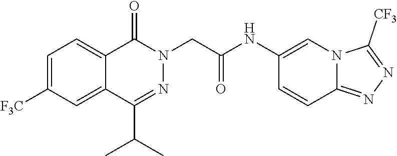 [SMILES: CC(C)c1nn(CC(=O)Nc2ccc3nnc(C(F)(F)F)n3c2)c(=O)c2ccc(C(F)(F)F)cc12] | X87 |
| 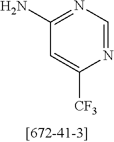 [SMILES: Nc1cc(C(F)(F)F)ncn1] | 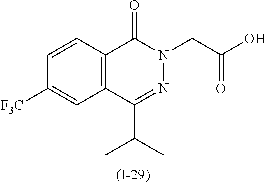 | 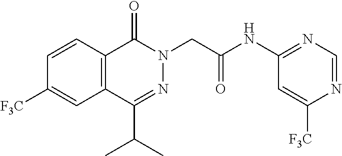 [SMILES: CC(C)c1nn(CC(=O)Nc2cc(C(F)(F)F)ncn2)c(=O)c2ccc(C(F)(F)F)cc12] | X178 |

### Example A12

### Synthesis of 2-(4-isopropyl-1-oxo-6-(trifluoromethyl)phthalazin-2(1H)-yl)-N-(8-methyl-[1,2,4]triazolo[4,3-a]pyridin-6-yl)acetamide (X28)

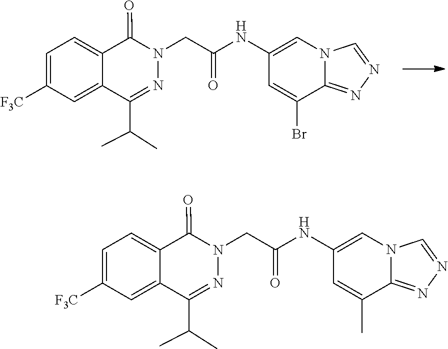 [SMILES: CC(C)c1nn(CC(=O)Nc2cc(Br)c3nncn3c2)c(=O)c2ccc(C(F)(F)F)cc12 ; Cc1cc(NC(=O)Cn2nc(C(C)C)c3cc(C(F)(F)F)ccc3c2=O)cn2cnnc12]

N-(8-Bromo-[1,2,4]triazolo[4,3-a]pyridin-6-yl)-2-(4-isopropyl-1-oxo-6-(trifluoromethyl)phthalazin-2(1H)-yl)acetamide X52 (50 mg, 0.1 mmol, 1 equiv) and bis(tri-tert-butylphosphine)palladium(0) [53199-31-8] (20 mg, 0.039 mmol, 40 mol %) were placed in a dry 8-mL MW vial. The vial was sealed and placed under nitrogen (3 vacuum/nitrogen cycles) and cooled to 0° C. with an ice-bath. Anhydrous THF (1 mL) was added, the mixture was allowed to stir for 2 minutes at 0° C. and a solution of MeZnCl [5158-46-3] (2 M in THF, 147 μL, 0.29 mmol, 3 equiv) was added dropwise over 2 min. The resulting solution was stirred vigorously at rt for 18 hours. The crude mixture was quenched by addition of 0.2M aqueous HCl (ca. 5 mL) and extracted with EtOAc (4×5 mL). The combined organic layers were dried over Na₂SO₄, filtered and concentrated in vacuo. The obtained residue was purified by preparative HPLC (Stationary phase: RP XBridge Prep C18 OBD-10 μm, 50×250 mm, Mobile phase: 0.25% NH₄HCO₃ solution in water, CH₃CN) to give X28 (20 mg, 46%) as a colorless solid.

### Example A13

### Synthesis of N-(8-cyano-[1,2,4]triazolo[4,3-a]pyridin-6-yl)-2-(4-isopropyl-1-oxo-6-trifluoromethyl)phthalazin-2(1H)-yl)acetamide (X72)

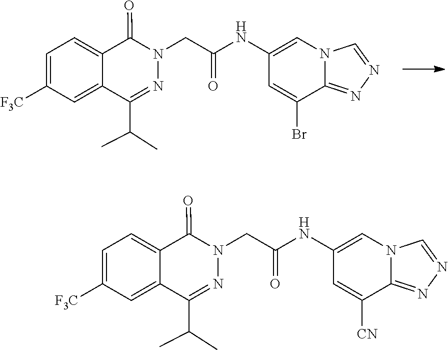 [SMILES: CC(C)c1nn(CC(=O)Nc2cc(Br)c3nncn3c2)c(=O)c2ccc(C(F)(F)F)cc12 ; CC(C)c1nn(CC(=O)Nc2cc(C#N)c3nncn3c2)c(=O)c2ccc(C(F)(F)F)cc12]

^{t}BuXPhos Pd G3 [1447963-75-8] (15.2 mg, 19 μmol), Zn(CN)₂ [557-21-1] (27 mg, 0.23 mmol) and N-(8-bromo-[1,2,4]triazolo[4,3-a]pyridin-6-yl)-2-(4-isopropyl-1-oxo-6-(trifluoromethyl)phthalazin-2(1H)-yl)acetamide X52 (65 mg, 0.13 mmol) were placed in a dry MW vial. The vial was sealed and placed under nitrogen (3 vacuum/nitrogen cycles) and 1.4 mL of degassed 1:2 mixture of THF/DI water was added. The vial was stirred vigorously at 55° C. for 18 h. The mixture was then sonicated until a fine suspension was observed and it was heated at 60° C. for further 24 hours.

The mixture was partitioned between DI water (10 mL) and DCM (10 mL). The organic layer was collected and the aqueous re-extracted with DCM (2×10 mL) then DCM/MeOH 95:5 (4×10 mL). The combined organic layers were dried over Na₂SO₄, filtered and concentrated in vacuo. The obtained residue was further purified by preparative HPLC (Stationary phase: RP XBridge Prep C18 OBD-5 μm, 50×250 mm, Mobile phase: 0.25% NH₄HCO₃ solution in water, CH₃CN) followed by preparative SFC (Stationary phase: Chiralpak Daicel ID 20×250 mm, Mobile phase: CO₂, EtOH+0.4 iPrNH₂) to give X72 (9 mg, 15%) as a pale tan solid.

Additional analogs were accessed using similar reaction conditions, using the appropriate reagent.

| Intermediate XX | Final compound | Number |
|---|---|---|
| 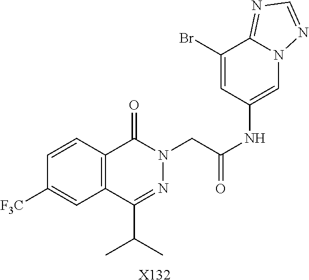 | 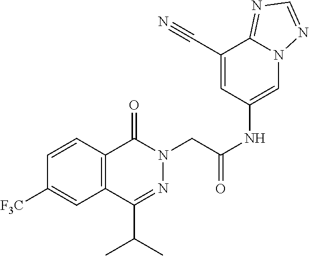 [SMILES: CC(C)c1nn(CC(=O)Nc2cc(C#N)c3ncnn3c2)c(=O)c2ccc(C(F)(F)F)cc12] | X162 |

### Example A14

### Synthesis of 2-(4-isopropyl-1-oxo-6-(trifluoromethyl)phthalazin-2(1H)-yl)-N-((1s,3s)-3-methoxy-3-methylcyclobutyl)acetamide (X29)

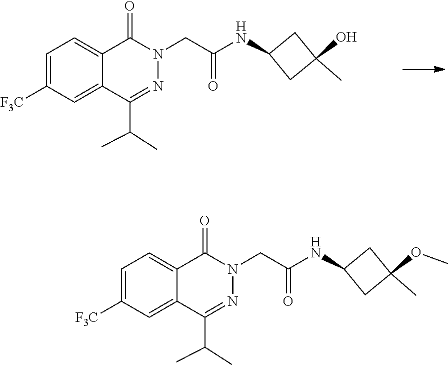 [SMILES: CC(C)c1nn(CC(=O)N[C@H]2C[C@@](C)(O)C2)c(=O)c2ccc(C(F)(F)F)cc12 ; CO[C@]1(C)C[C@H](NC(=O)Cn2nc(C(C)C)c3cc(C(F)(F)F)ccc3c2=O)C1]

N-((1s,3s)-3-Hydroxy-3-methylcyclobutyl)-2-(4-isopropyl-1-oxo-6-(trifluoromethyl)phthalazin-2(1H)-yl)acetamide X25 (108 mg, 0.27 mmol, 1 equiv) was placed in a 20-mL vial and dissolved in anhydrous DCM (10.4 mL). The solution was cooled to 0° C. in an ice-bath and placed under nitrogen. After 10 min at 0° C., 2,6-di-tert-butyl-4-methylpyridine [38222-83-2] (223.2 mg, 1.09 mmol, 4 equiv) and trimethyloxonium tetrafluoroborate [420-37-1] (120.6 mg, 0.82 mmol, 3 equiv) were added sequentially. The vial was flushed with nitrogen and sealed and the mixture stirred at 0° C. for 5 mdi then allowed to warm to m and stirred for 2 hours. The mixture was then quenched by addition of a saturated aqueous solution of NaHCO3 (ca. 5 mL) and extracted with DCM (2×8 mL). The combined organic extracts were concentrated and purified by FCC (Hept/EtOAc 4:1 to 0:1) to afford the title amide X29 (74 mg, 660%) as a colorless solid.

Additional analogs were accessed using similar reaction conditions, using the appropriate reagent.

| Intermediate XX | Final compound | Number |
|---|---|---|
| 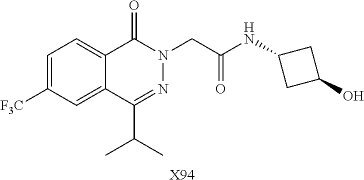 | 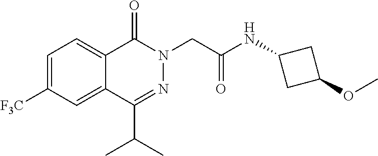 [SMILES: CO[C@H]1C[C@H](NC(=O)Cn2nc(C(C)C)c3cc(C(F)(F)F)ccc3c2=O)C1] | X105 |
| 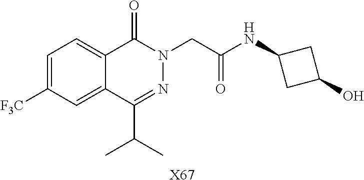 | 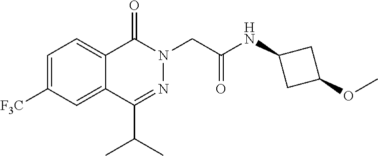 [SMILES: CO[C@H]1C[C@@H](NC(=O)Cn2nc(C(C)C)c3cc(C(F)(F)F)ccc3c2=O)C1] | X116 |
| 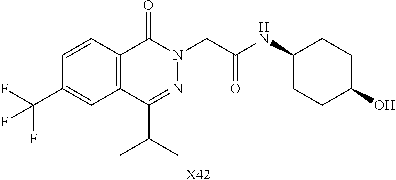 | 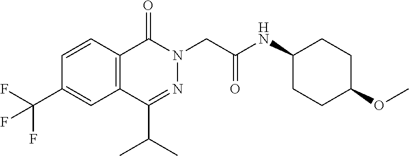 [SMILES: CO[C@H]1CC[C@@H](NC(=O)Cn2nc(C(C)C)c3cc(C(F)(F)F)ccc3c2=O)CC1] | X131 |
| 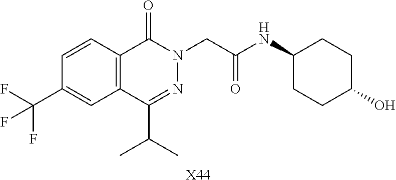 | 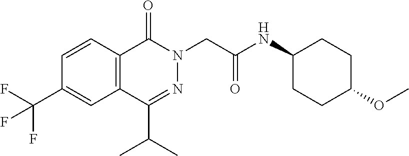 [SMILES: CO[C@H]1CC[C@H](NC(=O)Cn2nc(C(C)C)c3cc(C(F)(F)F)ccc3c2=O)CC1] | X177 |
| 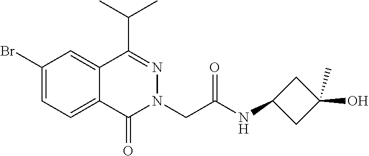 [SMILES: CC(C)c1nn(CC(=O)N[C@H]2C[C@@](C)(O)C2)c(=O)c2ccc(Br)cc12] | 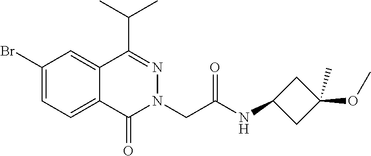 [SMILES: CO[C@]1(C)C[C@H](NC(=O)Cn2nc(C(C)C)c3cc(Br)ccc3c2=O)C1] | X-1088 |

### Example A15

### Synthesis of 2-(4-isopropyl-1-oxo-6-(trifluoromethyl)phthalazin-2(1H)-yl)-N-(3-methyl-3H-imidazo[4,5-b]pyridin-5-yl)acetamide (X47)

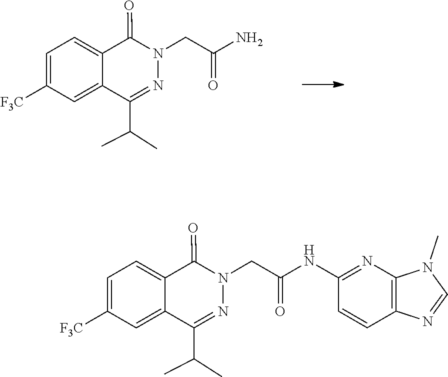 [SMILES: CC(C)c1nn(CC(N)=O)c(=O)c2ccc(C(F)(F)F)cc12 ; CC(C)c1nn(CC(=O)Nc2ccc3ncn(C)c3n2)c(=O)c2ccc(C(F)(F)F)cc12]

2-(4-Isopropyl-1-oxo-6-(trifluoromethyl)phthalazin-2(1H)-yl)acetamide (1-77) (20 mg, 0.119 mmol), 5-chloro-3-methyl-3H-imidazo[4,5-b]pyridine (1-135) (50 mg, 0.16 mmol), K₂CO₃ (36 mg, 0.26 mmol), CuI (1.2 mg, 0.0063 mmol) and trans-N,N-dimethylcyclohexane-1,2-diamine [67579-81-1] (1.2 mg, 8.4 μmol) were suspended in 2 mL of anhydrous 1,4-dioxane in a microwave vessel. The resulting mixture was degassed 5 min with nitrogen before being heated in the closed vessel at 170° C. during 18 h. The mixture was filtered over PTFE filter and washed with MeOH and purified by preparative HPLC (Stationary phase: RP XBridge Prep C18 OBD-10 μm, 50×250 mm, Mobile phase: 0.25% NH₄HCO₃ solution in water, MeOH) to afford X47 (9 mg, 17%) as white solid.

Additional analogs were accessed using similar reaction conditions, using the appropriate reagent.

Halide Amide substrate Final compound Number

| | | | Num- |
|---|---|---|---|
| Halide | Amide substrate | Final compound | ber |
| 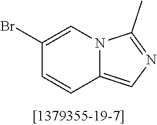 | 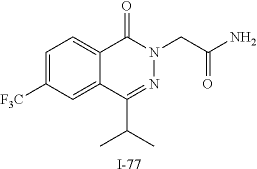 | 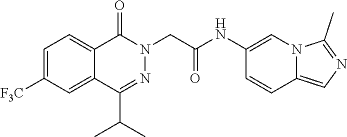 [SMILES: Cc1ncc2ccc(NC(=O)Cn3nc(C(C)C)c4cc(C(F)(F)F)ccc4c3=O)cn12] | X48 |
| 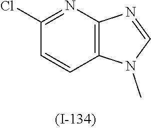 | 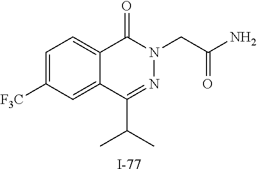 | 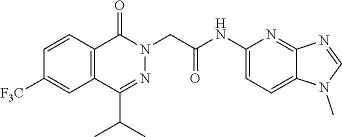 [SMILES: CC(C)c1nn(CC(=O)Nc2ccc3c(ncn3C)n2)c(=O)c2ccc(C(F)(F)F)cc12] | X160 |

### Example A16

### Synthesis of 2-(4-isopropyl-1-oxo-6-(trifluoromethyl)phthalazin-2(1H)-yl)-N-(2-(trifluoromethyl)imidazo[1,2-a]pyridin-6-yl)acetamide (X109)

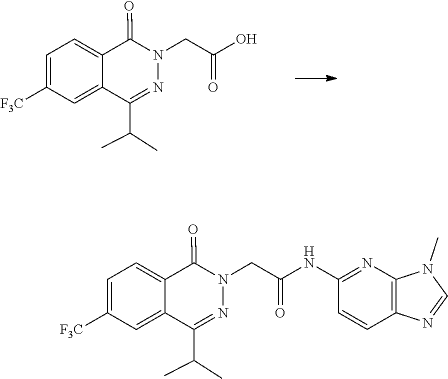 [SMILES: CC(C)c1nn(CC(=O)O)c(=O)c2ccc(C(F)(F)F)cc12 ; CC(C)c1nn(CC(=O)Nc2ccc3ncn(C)c3n2)c(=O)c2ccc(C(F)(F)F)cc12]

A mixture of carboxylic acid I-29) (100 mg, 0.32 mmol) and 1-methyl-1H-pyrazolo[3,4-b]pyridin-6-amine (I-131) (88.1 mg, 0.48 mmol) in dry pyridine [110-86-1] (8 ml) under nitrogen was sonicated for 10 min and then stirred for 40 min at rt. A 1M solution of titanium(IV) chloride in DCM [7550-45-0] (1.27 mL, 1.27 mmol) was added dropwise at rt. The mixture was stirred for at rt for 1 hour and then heated at 80° C. for 30 hours. The solvent was evaporated in vacuo and the crude mixture treated with 1M aqueous HCl until pH<7. The crude product was extracted with AcOEt and the combined organic layers were dried over MgSO₄, filtered and evaporated in vacuo. This crude compound was recrystallized from 15 mL of hot acetonitrile to yield X109 (90 mg, 64%) as a colorless solid.

### Example A17

### Synthesis of tert-butyl (3S,4R)-3-(2-(6-bromo-4-isopropyl-1-oxophthalazin-2(1H)-yl)acetamido)-4-hydroxypiperidine-1-carboxylate I-1132

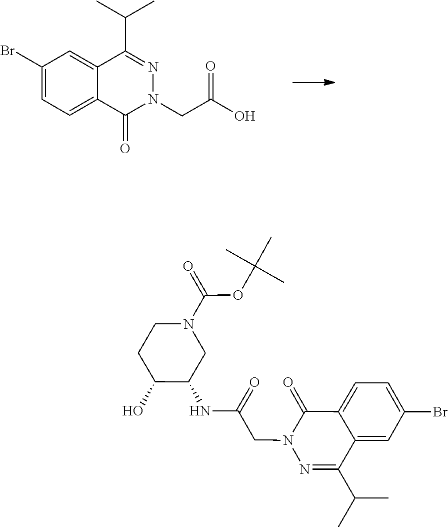 [SMILES: CC(C)c1nn(CC(=O)O)c(=O)c2ccc(Br)cc12 ; CC(C)c1nn(CC(=O)N[C@H]2CN(C(=O)OC(C)(C)C)CC[C@H]2O)c(=O)c2ccc(Br)cc12]

HATU [148893-10-1] (1.17 g, 3.08 mmol) was added to a stirred solution of 2-(6-bromo-4-isopropyl-1-oxophthalazin-2(1H)-yl)acetic acid I-1131 (500 mg, 1.54 mmol) in DMF (3.5 mL) at rt followed by the addition of tert-butyl (3S,4R)-3-amino-4-hydroxypiperidine-1-carboxylate [1820579-78-9] (379.54 mg, 1.75 mmol) and DIPEA [7087-68-5] (2.65 mL, 0.75 g/mL, 15.38 mmol). The mixture was stirred at RT for 18 h. Water is added, and reaction mixture is stirred for further 30 minutes, then the white solid is filtered and washed with water. The solid is dried in the oven at 50° C. overnight to obtain tert-butyl (3S,4R)-3-(2-(6-bromo-4-isopropyl-1-oxophthalazin-2(1H)-yl)acetamido)-4-hydroxypiperidine-1-carboxylate I-1132 (570 mg, yield 67%) as a white solid.

| | | | Num- |
|---|---|---|---|
| Reagent | Intermediate | Final cpd | ber |
| 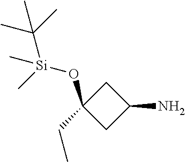 | 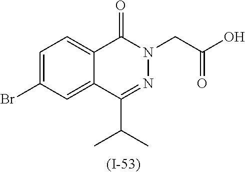 | 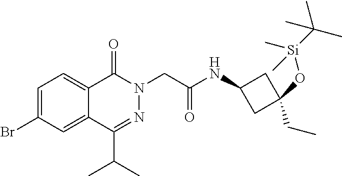 [SMILES: CC[C@]1(O[Si](C)(C)C(C)(C)C)C[C@@H](NC(=O)Cn2nc(C(C)C)c3cc(Br)ccc3c2=O)C1] | X- 1089 |

### Synthesis of 2-(6-bromo-4-isopropyl-1-oxophthalazin-2(1H)-yl)-N-((1r,3s)-3-ethyl-3-hydroxycyclobutyl)acetamide X-1090

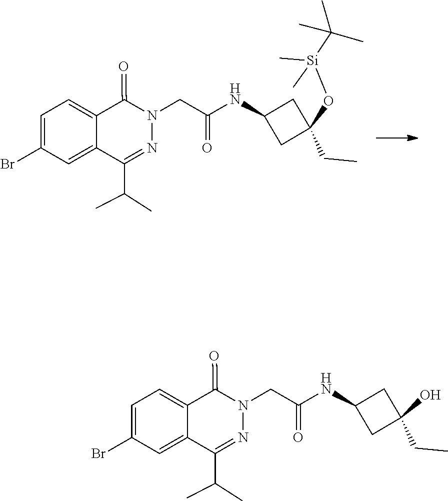 [SMILES: CC[C@]1(O[Si](C)(C)C(C)(C)C)C[C@@H](NC(=O)Cn2nc(C(C)C)c3cc(Br)ccc3c2=O)C1 ; CC[C@]1(O)C[C@@H](NC(=O)Cn2nc(C(C)C)c3cc(Br)ccc3c2=O)C1]

To a mixture of 2-(6-bromo-4-isopropyl-1-oxophthalazin-2(1H)-yl)-N-((1r,3s)-3-((tert-butyldimethylsilyl)oxy)-3-ethylcyclobutyl)acetamide X-1089 (120 mg, 0.2236 mmol) in DCM (5 mL), TFA [76-05-1] (0.1711 mL, 1.49 g/mL, 2.2364 mmol) was added at rt. The mixture was stirred for 16 h. The crude was evaporated in vacuo and diluted with sat. Na2CO3 and the mixture was stirred at RT for 30 min. Then, the mixture was extracted with DCM, the organic layer was separated, and the aqueous phase was further extracted with additional DCM (2×). The combined organic layers were dried (Na2SO4), filtered and the solvents evaporated in vacuo. The crude product was purified by flash column chromatography (Silica MeOH in DCM 0/100 to 3/97). The desired fractions were collected, the solvent evaporated in vacuo to yield 2-(6-bromo-4-isopropyl-1-oxophthalazin-2(1H)-yl)-N-((1 r, 3s)-3-ethyl-3-hydroxycyclobutyl)acetamide X-1090 (57 mg, yield 601) as white solid.

Characterising Data—LC-MS and Melting Point

LCMS: [M+H]⁺ means the protonated mass of the free base of the compound, Rₜ means retention time (in minutes), method refers to the method used for LCMS.

| | | | | | LCMS |
|---|---|---|---|---|---|
| Final Cpd | M.p. ° C. (Method) | | [M + H]⁺ | Rₜ | Method |
| 2 | 265.1 | (A) | 388 | 2.90 | 1 |
| 4 | 246.6 | (A) | 324 | 2.64 | 1 |
| 5 | 193.2 | (A) | 350 | 2.96 | 1 |
| 6 | 179.7 | (A) | 352 | 3.18 | 1 |
| 7 | 186.5 | (A) | 338 | 2.95 | 1 |
| 9 | 251.7 | (A) | 378 | 3.05 | 1 |
| 10 | 196.74 | (B) | 354 | 2.10 | 2 |
| 11 | n.t. | | 245 | 1.65 | 3 |
| 12 | 215.30 | (B) | 310 | 1.52 | 4 |
| 13 | | | 309 | 1.52 | 4 |
| 14 | 213.52 | (B) | 272 | 1.52 | 4 |
| 16 | 253.3 | (A) | 360 | 2.71 | 1 |
| 17 | 204.9 | (A) | 366 | 2.43 | 1 |
| 18 | 208.2 | (A) | 395 | 2.58 | 1 |
| 19 | 218.3 | (A) | 384 | 2.74 | 1 |
| 20 | 193.2 | (A) | 358 | 2.86 | 1 |
| 21 | 199.9 | (A) | 356 | 2.65 | 1 |
| 22 | 191.5 | (A) | 344 | 2.63 | 1 |
| 24 | 245.0 | (A) | 408 | 2.60 | 1 |
| 26 | 229.9 | (A) | 344 | 2.38 | 1 |
| 27 | 209.8 | (A) | 370 | 2.67 | 1 |
| 28 | 188.1 | (A) | 372 | 2.88 | 1 |
| 29 | 199.9 | (A) | 358 | 2.65 | 1 |
| 30 | 243.4 | (A) | 398 | 2.75 | 1 |
| X1 | NA | | 432 | 1.71 | 3 |
| X2 | 246.08 (major)/ | | 432 | 1.75 | 4 |
| | 258.21 (minor) (B) | | | | |
| X3 | NA | | 450 | 1.86 | 3 |
| X4 | 236.7 | (A) | 396 | 2.84 | 1 |
| X5 | 276.97 | (B) | 460 | 1.81 | 3 |
| X6 | 177.95 | (B) | 441 | 1.81 | 4 |
| X8 | >300 | (A) | 417 | 2.76 | 1 |
| X9 | N.D. | | 425 | 1.98 | 4 |
| X10 | 194.52 | (B) | 437 | 2.15 | 4 |
| X11 | — | | 466 | 2.26 | 6 |
| X12 | — | | 445 | 1.81 | 3 |
| X13 | 295.46 | | 431 | 1.76 | 4 |
| X14 | ongoing | | 432 | 1.73 | 4 |
| X15 | 209.37 | (B) | 453 | 1.91 | 3 |
| X16 | 238.55 (minor)/ | | 431 | 1.86 | 4 |
| | 251.14 (major) (B) | | | | |
| X17 | — | | 416 | 2.15 | 5 |
| X18 | — | | 433 | 1.90 | 4 |
| X19 | 178.86 | | 439 | 1.57 | 3 |
| X20 | 222.09 | | 430 | 2.28 | 2 |
| X21 | — | | 393 | 1.80 | 4 |
| X22 | 232.18 | | 425 | 1.70 | 4 |
| X23 | 294.49 | | 429 | 1.67 | 4 |
| X24 | 247.79 | (B) | 445 | 2.02 | 4 |
| X25 | 228.24 | (B) | 444 | 1.85 | 4 |
| X26 | — | | 449 | 1.24 | 7 |
| X27 | 247.46 | (B) | 444 | 1.90 | 4 |
| X28 | 274.69 | (B) | 445 | 1.84 | 4 |
| X29 | 162.05 | (A) | 412 | 1.96 | 4 |
| X30 | 246.7 | (A) | 378 | 2.77 | 1 |
| X31 | 237.79 | (B) | 430 | 1.83 | 4 |
| X32 | >300 | (A) | 439 | 2.85 | 1 |
| X33 | 272/30 | (B) | 422 | 1.81 | 3 |
| X34 | 276.8 | (A) | 430 | 2.93 | 1 |
| X35 | 218.22 | (B) | 452 | 1.95 | 4 |
| X36 | 208.3 | (A) | 406 | 2.86 | 1 |
| X37 | N.D. | | 444 | 2.07 | 3 |
| X38 | 233.38 | (B) | 431 | 1.87 | 4 |
| X39 | — | | 430 | 1.91 | 3 |
| X40 | 242.67 | (B) | 479 | 1.69 | 3 |
| X41 | N.D. | | 449 | 1.66 | 3 |
| X42 | N.D. | | 412 | 1.78 | 4 |
| X43 | — | | 445 | 1.81 | 3 |
| X44 | 235.29 | (B) | 412 | 1.80 | 4 |
| X45 | 219.21 | (B) | 451 | 1.83 | 4 |
| X46 | 197.64 | (B) | 426 | 1.88 | 3 |
| X47 | — | | 445 | 1.87 | 4 |
| X48 | — | | 444 | 1.92 | 3 |
| X49 | 220.00 | (B) | 445 | 1.91 | 4 |
| X50 | 236.7 | (A) | 400 | 3.15 | 1 |
| X51 | 210.92 | (B) | 436 | 1.95 | 4 |
| X52 | — | | 509/511 | 1.82 | 4 |
| X53 | 269.23 | (B) | 445 | 1.94 | 4 |
| X54 | 188.06 & 230.41 (B) | | 430 | 1.83 | 4 |
| X55 | 238.68 | (B) | 435 | 1.64 | 3 |
| X56 | 219.24 | (B) | 384 | 1.76 | 4 |
| X57 | 239.33 | (B) | 445 | 1.88 | 4 |
| X58 | >300 | (A) | 431 | 2.74 | 1 |
| X59 | 265 | (A) | 485 | 3.07 | 1 |
| X60 | — | | 437 | 1.94 | 4 |
| X61 | >300 | (A) | 487 | 2.74 | 1 |
| X62 | 241.74 & 362.78 (B) | | 466 | 1.83 | 3 |
| X63 | 241.31 | (B) | 466 | 1.89 | 4 |
| X64 | N.D. | | 498 | 2.10 | 4 |
| X66 | >300 | (A) | 433 | 2.25 | 1 |
| X67 | — | | 384 | 1.71 | 4 |
| X68 | 222.63 | (B) | 449 | 1.66 | 4 |
| X69 | 254.43 | (B) | 446 | 1.81 | 4 |
| X70 | — | | 485 | 1.73 | 4 |
| X71 | 233.7 | (A) | 449 | 2.97 | 1 |
| X72 | — | | 456 | 1.95 | 4 |
| X73 | 260.73 | (B) | 431 | 1.77 | 4 |
| X74 | — | | 444 | 1.81 | 4 |
| X75 | 203.3 | (A) | 400 | 2.26 | 1 |
| X76 | 304.93 | (B) | 485 | 1.74 | 4 |
| X77 | 163.33 | (B) | 436 | 2.09 | 4 |
| X78 | 178.70 | (B) | 426 | 0.96 | 8 |
| X79 | — | | 444 | 1.97 | 4 |
| X80 | | | | | |
| X81 | | | | | |
| X82 | 229.96 | (B) | 448 | 1.89 | 4 |
| X83 | — | | 485 | 1.73 | 4 |
| X84 | 171.4 | (A) | 454 | 2.75 | 1 |
| X85 | 198.1 | (A) | 452 | 3.07 | 1 |
| X86 | 177.19 | (B) | 398 | 1.82 | 4 |
| X87 | 211.35 | (B) | 500 | 2.06 | 3 |
| X88 | 224.18 | (B) | 455 | 1.04 | 8 |
| X89 | N.D. | | 435 | 1.62 | 4 |
| X90 | 215.56 | | 431 | 1.82 | 4 |
| X91 | N.D. | | 384 | 1.80 | 4 |
| X92 | — | | 444 | 1.88 | 4 |
| X93 | 247.78 | (B) | 420 | 1.70 | 4 |
| X-1027 | 193.2 | (A) | 364 | 2.80 | 1 |
| X-1028 | 253.4 | (A) | 358 | 3.17 | 1 |
| X-1029 | >300 | (A) | 397 | 2.81 | 1 |
| X-1088 | — | | 422/424 | 1.92 | 4 |
| X-1031 | — | | 472 | 1.77 | 3 |
| X-1080 | 218.3 | (A) | 410 | 2.07 | 1 |
| X-1081 | 258.5 | (A) | 404 | 2.28 | 1 |
| X-1046 | 286.9 | (A) | 406 | 2.71 | 1 |
| X-1048 | 171.4 | (A) | 419 | 3.32 | 1 |
| X-1049 | 183.2 | (A) | 387 | 3.30 | 1 |
| X-1050 | 158.1 | (A) | 380 | 3.73 | 1 |
| X-1082 | >300 | (A) | 443 | 2.08 | 1 |
| X-1054 | 158.0 | (A) | 421 | 2.67 | 1 |
| X-1090 | — | | 422 | 1.1 | |
| X-1018 | 164.8 | (A) | 424 | 2.53 | 1 |
| X-1017 | 258.4 | (A) | 457 | 2.56 | 1 |
| X-1016 | 209.9 | (A) | 418 | 2.87 | 1 |
| X-1062 | 189.9 | (A) | 394 | 2.92 | 1 |
| X-1063 | 268.5 | (A) | 427 | 2.92 | 1 |
| X-1051 | 226.6 | (A) | 420 | 3.42 | 1 |
| X-1030 | 231.00 | (B) | 396 | 1.83 | 4 |
| X-1032 | 169.86 | (B) | 435 | 1.93 | 4 |
| X-1033 | 177.71 & 233.44 (B) | | 454 | 2.00 | 3 |
| X-1022 | 216.6 | (A) | 406 | 2.69 | 1 |
| X-1023 | >300 | (A) | 439 | 2.72 | 1 |
| X-1070 | 214.9 | (A) | 362 | 2.62 | 1 |
| X-1002 | 290.3 | (A) | 396 | 2.75 | 1 |
| X-1003 | 223.3 | (A) | 428 | 3.00 | 1 |
| X-1064 | 139.7 | (A) | 388 | 2.93 | 1 |
| X-1071 | >300 | (A) | 395 | 2.66 | 1 |
| X-1004 | — | | 442/444 | 1.71 | 4 |
| X-1019 | >300 | (A) | 449/451 | 2.73 | 1 |
| X-1001 | 285.2 | (A) | 440 | 2.82 | 1 |
| X-1000 | >300 | (A) | 452 | 2.81 | 1 |
| X-1035 | 185.61 & 208.57 (B) | | 447 | 2.09 | 3 |
| X-1020 | 226.6 | (A) | 416/418 | 2.70 | 1 |
| X-1024 | 243.3 | (A) | 430 | 2.86 | 1 |
| X-1036 | 237.49 & 254.22 (B) | | 434 | 1.86 | 4 |
| X-1083 | 141.19 & 216.66 (B) | | | | |
| X-1021 | 290.2 | (A) | 440/442 | 2.84 | 1 |
| X-1084 | 241.81 | (B) | 465 | 1.88 | 3 |
| X-1065 | 243.5 | (A) | 418 | 3.08 | 1 |
| X-1073 | — | | 391 | 1.82 | 5 |
| X-1006 | 237.41 | (B) | 427 | 1.81 | 4 |
| X-1007 | — | | 422 | 1.70 | 4 |
| X-1075 | — | | 413 | 2.00 | 4 |
| X-1074 | — | | 421 | 1.67 | 4 |
| X-1066 | 189.73 | (B) | 417 | 1.64 | 4 |
| X-1008 | 220.53 | (B) | 404 | 1.73 | 4 |
| X-1067 | 214.59 | (B) | 426 | 1.79 | 4 |
| X-1009 | 214.11 | (B) | 406 | 1.83 | 4 |
| X-1085 | 145.85 & 266.38 (B) | | 428 | 1.71 | 4 |
| X-1086 | 236.53 | (B) | 406 | 1.86 | 4 |
| X-1012 | 206.6 | (A) | 418 | 3.37 | 1 |
| X-1010 | 235.83 | (B) | 424 | 1.74 | 4 |
| X-1011 | 239.24 | (B) | 423 | 1.85 | 4 |
| X-1076 | 184.50 | (B) | 423 | 1.72 | 3 |
| X-1077 | 185.30 | (B) | 422 | 1.83 | 3 |
| X-1087 | 254.67 | (B) | 424 | 1.76 | 4 |

Characterising Data—Compound+NMR

This is depicted in the following table:

| Compound | NMR |
|---|---|
| Final Cpd 1 | ¹H NMR (400 MHz, CDCl₃) δ ppm 1.38 (t, J = 7.4 Hz, 3 H) 3.00 (q, |
| | J = 7.4 Hz, 2 H) 5.04 (s, 2 H) 6.97 (dd, J = 9.8, 1.9 Hz, 1 H) 7.71 (br d, |
| | J = 9.9 Hz, 1 H) 7.94 (dd, J = 8.5, 1.8 Hz, 1 H) 8.01 (t, J = 1.9 Hz, 1 H) |
| | 8.37 (dd, J = 8.4, 1.9 Hz, 1 H) 8.75 (br s, 1 H) 9.15 (br s, 1 H) 9.20 (s, 1 H) |
| Final Cpd 2 | ¹H NMR (400 MHz, CDCl₃) δ ppm 1.38 (t, J = 7.4 Hz, 3 H) 2.99 (q, |
| | J = 7.4 Hz, 2 H) 5.05 (s, 2 H) 7.91 (dd, J = 8.5, 1.8 Hz, 1 H) 7.99 (d, |
| | J = 1.7 Hz, 1 H) 8.14 (dd, J = 5.7, 1.0 Hz, 1 H) 8.37 (d, J = 8.5 Hz, 1 H) |
| | 8.62 (d, J = 5.7 Hz, 1 H) 8.83 (br s, 1 H) 8.86 (s, 1 H) |
| Final Cpd 3 | ¹H NMR (400 MHz, CDCl₃) δ ppm 0.83-0.93 (m, 2 H) 1.15-1.23 |
| | (m, 2 H) 1.38 (t, J = 7.4 Hz, 3 H) 2.07-2.16 (m, 1 H) 3.01 (q, J = 7.4 |
| | Hz, 2 H) 5.05 (s, 2 H) 6.96 (dd, J = 9.7, 1.9 Hz, 1 H) 7.45 (dd, J = 8.3, |
| | 1.6 Hz, 1 H) 7.53 (d, J = 1.3 Hz, 1 H) 7.69 (d, J = 9.7 Hz, 1 H) 8.38 (d, |
| | J = 8.3 Hz, 1 H) 8.74 (s, 1 H) 9.19 (s, 1 H) 9.44 (s, 1 H) |
| Final Cpd 4 | ¹H NMR (400 MHz, CDCl₃) δ ppm 1.38 (t, J = 7.4 Hz, 3 H) 2.58 (s, 3 |
| | H) 3.01 (q, J = 7.4 Hz, 2 H) 5.06 (s, 2 H) 7.56-7.67 (m, 2 H) 8.15 |
| | (dd, J = 5.7, 1.2 Hz, 1 H) 8.40 (d, J = 8.7 Hz, 1 H) 8.61 (d, J = 5.8 Hz, 1 |
| | H) 8.84 (d, J = 1.0 Hz, 1 H) 9.04 (br s, 1 H) |
| Final Cpd 5 | ¹H NMR (300 MHz, CDCl₃) δ ppm 0.80-0.93 (m, 2 H) 1.10-1.22 |
| | (m, 2 H) 1.38 (t, J = 7.4 Hz, 3 H) 2.06-2.18 (m, 1 H) 3.00 (q, J = 7.4 |
| | Hz, 2 H) 5.05 (s, 2 H) 7.42 (br d, J = 8.4 Hz, 1 H) 7.51 (s, 1 H) 8.15 |
| | (br d, J = 5.6 Hz, 1 H) 8.38 (d, J = 8.2 Hz, 1 H) 8.61 (d, J = 5.8 Hz, 1 H) |
| | 8.84 (s, 1 H) 9.03 (br s, 1 H) |
| Final Cpd 6 | ¹H NMR (400 MHz, CDCl₃) δ ppm 1.35 (d, J = 6.9 Hz, 6 H) 1.39 (t, |
| | J = 7.4 Hz, 3 H) 3.03 (q, J = 7.4 Hz, 2 H) 3.13 (spt, J = 6.9 Hz, 1 H) |
| | 5.06 (s, 2 H) 7.65 (d, J = 1.5 Hz, 1 H) 7.69 (dd, J = 8.3, 1.5 Hz, 1 H) |
| | 8.15 (dd, J = 5.8, 1.3 Hz, 1 H) 8.44 (d, J = 8.2 Hz, 1 H) 8.61 (d, J = 5.7 |
| | Hz, 1 H) 8.84 (d, J = 1.1 Hz, 1 H) 8.99 (br s, 1 H) |
| Final Cpd 7 | ¹H NMR (400 MHz, CDCl₃) δ ppm 1.34 (t, J = 7.6 Hz, 3 H) 1.38 (t, |
| | J = 7.4 Hz, 3 H) 2.87 (q, J = 7.6 Hz, 2 H) 3.02 (q, J = 7.4 Hz, 2 H) 5.06 |
| | (s, 2 H) 7.62-7.68 (m, 2 H) 8.15 (dd, J = 5.7, 1.3 Hz, 1 H) 8.43 (d, |
| | J = 8.2 Hz, 1 H) 8.61 (d, J = 5.8 Hz, 1 H) 8.84 (d, J = 1.1 Hz, 1 H) 9.00 |
| | (br s, 1 H) |
| Final Cpd 8 | ¹H NMR (400 MHz, DMSO-d₆) δ ppm 1.29 (d, J = 6.6 Hz, 6 H) 3.77 |
| | (spt, J = 6.7 Hz, 1 H) 5.02 (s, 2 H) 7.31 (dd, J = 9.8, 1.9 Hz, 1 H) 7.79 |
| | (dt, J = 9.7, 1.0 Hz, 1 H) 8.22 (dd, J = 8.3, 1.2 Hz, 1 H) 8.42 (s, 1 H) |
| | 8.52 (d, J = 8.4 Hz, 1 H) 9.19-9.21 (m, 1 H) 9.23 (d, J = 0.7 Hz, 1 H) |
| | 10.57 (s, 1 H) |
| Final Cpd 9 | ¹H NMR (400 MHz, CDCl₃) δ ppm 1.41 (t, J = 7.4 Hz, 3 H) 3.07 (q, |
| | J = 7.4 Hz, 2 H) 5.08 (s, 2 H) 8.02 (dd, J = 8.3, 1.3 Hz, 1 H) 8.11 (s, 1 |
| | H) 8.13 (dd, J = 5.8, 1.2 Hz, 1 H) 8.63 (d, J = 5.8 Hz, 1 H) 8.65 (d, |
| | J = 8.4 Hz, 1 H) 8.74 (br s, 1 H) 8.87 (d, J = 1.1 Hz, 1 H) |
| Final Cpd 10 | ¹H NMR (400 MHz, DMSO-d₆) δ ppm 1.27 (d, J = 6.6 Hz, 6 H) 3.61 |
| | (spt, J = 6.7 Hz, 1 H) 3.98 (s, 3 H) 5.00 (s, 2 H) 7.41 (d, J = 2.4 Hz, 1 |
| | H) 7.46 (dd, J = 8.8, 2.4 Hz, 1 H) 7.98 (dd, J = 5.8, 1.2 Hz, 1 H) 8.22 |
| | (d, J = 8.8 Hz, 1 H) 8.65 (d, J = 5.9 Hz, 1 H) 8.91 (d, J = 0.9 Hz, 1 H) |
| | 11.26 (br s, 1 H) |
| Final Cpd 11 | ¹H NMR (400 MHz, DMSO-d₆) δ ppm 1.26 (t, J = 7.5 Hz, 3 H) 2.98 |
| | (q, J = 7.3 Hz, 2 H) 3.98 (s, 3 H) 5.00 (s, 2 H) 7.35 (d, J = 2.4 Hz, 1 H) |
| | 7.45 (dd, J = 8.8, 2.4 Hz, 1 H) 7.97 (dd, J = 5.8, 1.2 Hz, 1 H) 8.20 (d, |
| | J = 8.8 Hz, 1 H) 8.64 (d, J = 5.7 Hz, 1 H) 8.90 (d, J = 0.9 Hz, 1 H) |
| | 11.26 (br s, 1 H) |
| Final Cpd 12 | ¹H NMR (400 MHz, CDCl₃) δ ppm 1.38 (t, J = 7.5 Hz, 3 H) 3.03 (q, |
| | J = 7.5 Hz, 2 H) 5.08 (s, 2 H) 7.79-7.83 (m, 1 H) 7.84-7.90 (m, 2 |
| | H) 8.15 (dd, J = 5.7, 1.3 Hz, 1 H) 8.51-8.54 (m, 1 H) 8.61 (d, J = 5.7 |
| | Hz, 1 H) 8.84 (d, J = 1.1 Hz, 1 H) 9.03 (br s, 1 H) |
| Final Cpd 13 | ¹H NMR (400 MHz, CDCl₃) δ ppm 1.38 (t, J = 7.4 Hz, 3 H) 3.03 (q, |
| | J = 7.4 Hz, 2 H) 5.07 (s, 2 H) 7.19 (dd, J = 8.3, 4.8 Hz, 1 H) 7.77- |
| | 7.84 (m, 1 H) 7.84-7.90 (m, 2 H) 8.07-8.12 (m, 1 H) 8.29 (dd, |
| | J = 4.8, 1.4 Hz, 1 H) 8.51 (dt, J = 7.8, 1.0 Hz, 1 H) 8.57 (d, J = 2.2 Hz, |
| | 1 H) 9.11 (br s, 1 H) |
| Final Cpd 14 | ¹H NMR (400 MHz, DMSO-d₆) δ ppm 0.36-0.49 (m, 2 H) 0.55- |
| | 0.68 (m, 2 H) 1.25 (t, J = 7.5 Hz, 3 H) 2.64 (tq, J = 7.4, 3.9 Hz, 1 H) |
| | 2.97 (q, J = 7.6 Hz, 2 H) 4.64 (s, 2 H) 7.84-7.89 (m, 1 H) 7.95 (td, |
| | J = 7.6, 1.4 Hz, 1 H) 8.00-8.05 (m, 1 H) 8.15 (br d, J = 3.7 Hz, 1 H) |
| | 8.25-8.31 (m, 1 H) |
| Final Cpd 15 | ¹H NMR (400 MHz, DMSO-d₆) δ ppm 1.28 (d, J = 6.8 Hz, 6 H) 3.76 |
| | (spt, J = 6.8 Hz, 1 H) 5.07 (s, 2 H) 7.98 (dd, J = 5.7, 1.3 Hz, 1 H) 8.21 |
| | (dd, J = 8.4, 1.3 Hz, 1 H) 8.41 (s, 1 H) 8.51 (d, J = 8.4 Hz, 1 H) 8.66 |
| | (d, J = 5.7 Hz, 1 H) 8.92 (d, J = 0.9 Hz, 1 H) 11.31 (br s, 1 H) |
| Final Cpd 16 | ¹H NMR (400 MHz, CDCl₃) δ ppm 1.40 (t, J = 7.4 Hz, 3 H) 3.06 (q, |
| | J = 7.4 Hz, 2 H) 5.09 (s, 2 H) 6.83 (t, J = 55.8 Hz, 1 H) 7.92 (d, J = 8.2 |
| | Hz, 1 H) 7.99 (s, 1 H) 8.14 (dd, J = 5.8, 1.2 Hz, 1 H) 8.61 (d, J = 3.0 |
| | Hz, 1 H) 8.63 (s, 1 H) 8.86 (d, J = 1.0 Hz, 1 H) 8.89 (br s, 1 H) |
| Final Cpd 17 | ¹H NMR (400 MHz, CDCl₃) δ ppm 1.33-1.41 (m, 6 H) 1.98 (s, 1 |
| | H) 1.98-2.07 (m, 2 H) 2.46-2.56 (m, 2 H) 3.03 (q, J = 7.4 Hz, 2 H) |
| | 4.00 (sxt, J = 7.9 Hz, 1 H) 4.85 (s, 2 H) 6.38 (br d, J = 7.0 Hz, 1 H) |
| | 6.83 (t, J = 55.8 Hz, 1 H) 7.90 (d, J = 8.2 Hz, 1 H) 7.97 (s, 1 H) 8.57 |
| | (d, J = 8.2 Hz, 1 H) |
| Final Cpd 18 | ¹H NMR (400 MHz, CDCl₃) δ ppm 1.29-1.41 (m, 6 H) 1.92-2.07 |
| | (m, 3 H) 2.44-2.57 (m, 2 H) 2.96 (q, J = 7.4 Hz, 2 H) 4.00 (sxt, |
| | J = 7.9 Hz, 1 H) 4.82 (s, 2 H) 6.38 (br d, J = 6.5 Hz, 1 H) 7.88 (dd, |
| | J = 8.5, 1.8 Hz, 1 H) 7.97 (d, J = 1.7 Hz, 1 H) 8.32 (d, J = 8.6 Hz, 1 H) |
| Final Cpd 19 | ¹H NMR (400 MHz, CDCl₃) δ ppm 1.36 (s, 3 H) 1.38 (t, J = 7.4 Hz, 3 |
| | H) 1.96-2.09 (m, 3 H) 2.43-2.58 (m, 2 H) 3.04 (q, J = 7.4 Hz, 2 H) |
| | 4.01 (sxt, J = 7.9 Hz, 1 H) 4.85 (s, 2 H) 6.38 (br d, J = 7.0 Hz, 1 H) |
| | 7.99 (dd, J = 8.4, 1.1 Hz, 1 H) 8.08 (s, 1 H) 8.60 (d, J = 8.4 Hz, 1 H) |
| Final Cpd 20 | ¹H NMR (400 MHz, DMSO-d₆) δ ppm 1.38-1.33 (m, 12 H) 2.05- |
| | 1.96 (m, 2 H) 2.10 (s, 1 H) 2.55-2.46 (m, 2 H) 2.99 (p, J = 7.4 Hz, 2 |
| | H) 3.12 (dt, J = 10.8, 6.9 Hz, 1 H) 3.98 (dd, J = 15.8, 7.9 Hz, 1 H) 4.84 |
| | (s, 2 H) 6.54 (d, J = 7.2 Hz, 1 H) 7.63 (d, J = 1.4 Hz, 1 H) 7.67 (dd, |
| | J = 8.3, 1.6 Hz, 1 H) 8.39 (d, J = 8.2 Hz, 1 H) |
| Final Cpd 21 | ¹H NMR (400 MHz, CDCl₃) δ ppm 0.89-0.83 (m, 2 H) 1.21-1.13 |
| | (m, 2 H) 1.40-1.30 (m, 6 H) 2.05-1.97 (m, 2 H) 2.10 (td, J = 8.4, |
| | 4.2 Hz, 1 H) 2.55-2.44 (m, 2 H) 3.04-2.91 (m, 2 H) 3.98 (dd, |
| | J = 15.8, 7.9 Hz, 1 H) 4.83 (s, 2 H) 6.53 (d, J = 5.9 Hz, 1H) 7.41 (dd, |
| | J = 8.3, 1.6 Hz, 1 H) 7.49 (s, 1 H) 8.33 (d, J = 8.3 Hz, 1 H) |
| Final Cpd 22 | ¹H NMR (400 MHz, CDCl₃) δ ppm 1.38-1.31 (m, 9 H) 2.05-1.99 |
| | (m, 2 H) 2.54-2.45 (m, 2 H) 2.91-2.80 (m, 2 H) 3.05-2.95 (m, 2 |
| | H) 3.99 (dd, J = 15.8, 7.9 Hz, 1 H) 4.84 (s, 2 H) 6.51 (d, J = 5.7 Hz, 1 |
| | H) 7.63 (d, J = 10.3 Hz, 2 H) 8.38 (d, J = 8.1 Hz, 1 H) |
| Final Cpd 23 | ¹H NMR (400 MHz, DMSO-d₆) δ ppm 0.87-0.95 (m, 2 H) 1.08- |
| | 1.18 (m, 2 H) 1.26 (d, J = 6.6 Hz, 6 H) 2.16-2.28 (m, 1 H) 3.64 (spt, |
| | J = 6.8 Hz, 1 H) 5.00 (s, 2 H) 7.52 (dd, J = 8.4, 1.8 Hz, 1 H) 7.76 (d, |
| | J = 1.3 Hz, 1 H) 7.98 (dd, J = 5.7, 1.3 Hz, 1 H) 8.16 (d, J = 8.1 Hz, 1 H) |
| | 8.65 (d, J = 5.7 Hz, 1 H) 8.91 (d, J = 0.9 Hz, 1 H) 11.26 (s, 1 H) |
| Final Cpd 24 | ¹H NMR (400 MHz, CDCl₃) δ ppm 1.13-1.26 (m, 2 H) 1.30-1.45 |
| | (m, 6 H) 1.87-2.09 (m, 4 H) 2.96 (q, J = 7.4 Hz, 2 H) 3.53-3.64 (m, |
| | 1 H) 3.77 (tdt, J = 11.3, 11.3, 7.6, 3.8, 3.8 Hz, 1 H) 4.81 (s, 2 H) 6.01 |
| | (br d, J = 7.2 Hz, 1 H) 7.88 (dd, J = 8.5, 1.8 Hz, 1 H) 7.96 (d, J = 1.7 |
| | Hz, 1 H) 8.32 (d, J = 8.5 Hz, 1 H) |
| Final Cpd 25 | ¹H NMR (400 MHz, DMSO-d₆) δ ppm 1.21 (s, 3 H) 1.26 (d, J = 6.7 |
| | Hz, 6 H) 1.89-2.00 (m, 2 H) 2.17-2.27 (m, 2 H) 3.67-3.84 (m, 2 |
| | H) 4.69 (s, 2 H) 4.98 (s, 1 H) 8.19 (dd, J = 8.5, 1.3 Hz, 1 H) 8.28 (d, |
| | J = 7.2 Hz, 1 H) 8.39 (s, 1 H) 8.49 (d, J = 8.4 Hz, 1 H) |
| Final Cpd 26 | ¹H NMR (400 MHz, DMSO-d₆) δ ppm 1.11-1.31 (m, 7 H) 1.70- |
| | 1.84 (m, 4 H) 2.54 (s, 3 H) 2.94 (q, J = 7.4 Hz, 2 H) 3.32-3.41 (m, 1 |
| | H) 3.43-3.52 (m, 1 H) 4.51 (d, J = 4.4 Hz, 1 H) 4.64 (s, 2 H) 7.68 |
| | (dd, J = 8.2, 1.0 Hz, 1 H) 7.81 (s, 1 H) 7.92 (d, J = 7.7 Hz, 1 H) 8.16 |
| | (d, J = 8.1 Hz, 1 H) |
| Final Cpd 27 | ¹H NMR (400 MHz, CDCl₃) δ ppm 0.81-0.92 (m, 2 H) 1.08-1.24 |
| | (m, 4 H) 1.27-1.46 (m, 6 H) 1.84-2.04 (m, 4 H) 2.05-2.14 (m, 1 |
| | H) 2.97 (q, J = 7.4 Hz, 2 H) 3.49-3.64 (m, 1 H) 3.76 (tdt, J = 11.3, |
| | 11.3, 7.7, 3.9, 3.9 Hz, 1 H) 4.82 (s, 2 H) 6.16 (br d, J = 7.8 Hz, 1 H) |
| | 7.40 (dd, J = 8.3, 1.6 Hz, 1 H) 7.49 (d, J = 1.3 Hz, 1 H) 8.33 (d, J = 8.3 |
| | Hz, 1 H) |
| Final Cpd 28 | ¹H NMR (400 MHz, CDCl₃) δ ppm 1.11-1.24 (m, 2 H) 1.29-1.44 |
| | (m, 12 H) 1.88-2.04 (m, 4 H) 3.00 (q, J = 7.4 Hz, 2 H) 3.12 (spt, |
| | J = 6.9 Hz, 1 H) 3.52-3.62 (m, 1 H) 3.76 (tdt, J = 11.4, 11.4, 7.6, 3.9, |
| | 3.9 Hz, 1 H) 4.83 (s, 2 H) 6.16 (br d, J = 7.7 Hz, 1 H) 7.63 (d, J = 1.4 |
| | Hz, 1 H) 7.67 (dd, J = 8.3, 1.5 Hz, 1 H) 8.39 (d, J = 8.2 Hz, 1 H) |
| Final Cpd 29 | ¹H NMR (400 MHz, CDCl₃) δ ppm 1.09-1.24 (m, 2 H) 1.29-1.44 |
| | (m, 9 H) 1.86-2.05 (m, 4 H) 2.86 (q, J = 7.6 Hz, 2 H) 2.99 (q, J = 7.4 |
| | Hz, 2 H) 3.51-3.63 (m, 1 H) 3.76 (tdt, J = 11.4, 11.4.7.6, 3.9, 3.9 |
| | Hz, 1 H) 4.83 (s, 2 H) 6.16 (br d, J = 7.2 Hz, 1 H) 7.59-7.66 (m, 2 |
| | H) 8.38 (d, J = 8.1 Hz, 1 H) |
| Final Cpd 30 | ¹H NMR (400 MHz, CDCl₃) δ ppm 1.14-1.28 (m, 2 H) 1.32-1.45 |
| | (m, 2 H) 1.38 (t, J = 7.4 Hz, 3 H) 1.90-2.07 (m, 4 H) 3.04 (q, J = 7.4 |
| | Hz, 2 H) 3.53-3.65 (m, 1 H) 3.78 (tdt, J = 11.4, 11.4, 7.6, 4.0, 4.0 |
| | Hz, 1 H) 4.84 (s, 2 H) 5.97 (br d, J = 6.6 Hz, 1 H) 7.99 (dd, J = 8.3, 1.3 |
| | Hz, 1 H) 8.08 (br s, 1 H) 8.60 (d, J = 8.4 Hz, 1 H); 1H exchanged |
| Final Cpd 31 | ¹H NMR (400 MHz, DMSO-d₆) δ ppm 0.86-0.98 (m, 2 H) 1.08- |
| | 1.19 (m, 2 H) 1.27 (d, J = 6.8 Hz, 6 H) 2.18-2.28 (m, 1 H) 3.65 (spt, |
| | J = 6.8 Hz, 1 H) 4.96 (s, 2 H) 7.32 (dd, J = 9.8, 1.9 Hz, 1 H) 7.52 (dd, |
| | J = 8.4, 1.6 Hz, 1 H) 7.78 (d, J = 1.4 Hz, 1 H) 7.80 (dt, J = 9.7, 0.9 Hz, |
| | 1 H) 8.17 (d, J = 8.3 Hz, 1 H) 9.21 (dd, J = 1.7, 1.0 Hz, 1 H) 9.24 (d, |
| | J = 0.7 Hz, 1 H) 10.57 (br s, 1 H) |
| Final Cpd 32 | ¹H NMR (400 MHz, DMSO-d₆) δ ppm 0.84-0.96 (m, 2 H) 1.07- |
| | 1.17 (m, 2 H) 1.21 (s, 3 H) 1.25 (d, J = 6.8 Hz, 6 H) 1.90-1.99 (m, 2 |
| | H) 2.17-2.27 (m, 3 H) 3.61 (spt, J = 6.7 Hz, 1 H) 3.71-3.83 (m, 1 |
| | H) 4.63 (s, 2 H) 4.98 (s, 1 H) 7.49 (dd, J = 8.4, 1.6 Hz, 1 H) 7.73 (d, |
| | J = 1.6 Hz, 1 H) 8.14 (d, J = 8.3 Hz, 1 H) 8.25 (d, J = 7.2 Hz, 1 H) |
| Final Cpd 33 | ¹H NMR (400 MHz, DMSO-d₆) δ ppm 1.29 (d, J = 6.7 Hz, 6 H) 1.31 |
| | (d, J = 6.9 Hz, 6 H) 3.18 (spt, J = 6.9 Hz, 1 H) 3.67 (spt, J = 6.7 Hz, 1 |
| | H) 4.97 (s, 2 H) 7.32 (dd, J = 9.8, 1.9 Hz, 1 H) 7.77-7.83 (m, 2 H) |
| | 7.91 (dt, J = 1.4, 0.7 Hz, 1 H) 8.24 (d, J = 8.2 Hz, 1 H) 9.20 (dd, J = 1.8, |
| | 1.0 Hz, 1 H) 9.24 (d, J = 0.8 Hz, 1 H) 10.55 (br s, 1 H) |
| Final Cpd 34 | ¹H NMR (400 MHz, DMSO-d₆) δ ppm 1.21 (s, 3 H) 1.26 (d, J = 6.8 |
| | Hz, 6 H) 1.30 (d, J = 6.9 Hz, 6 H) 1.90-1.98 (m, 2 H) 2.18-2.25 (m, |
| | 2 H) 3.16 (spt, J = 7.1 Hz, 1 H) 3.62 (spt, J = 6.7 Hz, 1 H) 3.77 (sxt, |
| | J = 8.1 Hz, 1 H) 4.64 (s, 2 H) 4.97 (s, 1 H) 7.78 (dd, J = 8.3, 1.5 Hz, 1 |
| | H) 7.86 (d, J = 1.1 Hz, 1 H) 8.21 (d, J = 8.4 Hz, 1 H) 8.21-8.26 (m, 1 H) |
| Final Cpd 35 | ¹H NMR (400 MHz, DMSO-d₆) δ ppm 1.28 (d, J = 6.7 Hz, 6 H) 1.31 |
| | (d, J = 6.9 Hz, 6 H) 3.18 (spt, J = 6.9 Hz, 1 H) 3.66 (spt, J = 6.7 Hz, 1 |
| | H) 5.02 (s, 2 H) 7.80 (dd, J = 8.3, 1.5 Hz, 1 H) 7.90 (d, J = 1.0 Hz, 1 |
| | H) 7.98 (dd, J = 5.8, 1.3 Hz, 1 H) 8.22 (d, J = 8.2 Hz, 1 H) 8.66 (d, |
| | J = 6.0 Hz, 1 H) 8.92 (d, J = 0.8 Hz, 1 H) 11.29 (br s, 1 H) |
| Final Cpd 36 | ¹H NMR (400 MHz, DMSO-d₆) δ ppm 1.26 (d, J = 6.7 Hz, 6 H) 3.65 |
| | (spt, J = 6.8 Hz, 1 H) 4.98 (s, 2 H) 7.31 (dd, J = 9.7, 1.9 Hz, 1 H) 7.80 |
| | (dt, J = 9.8, 0.8 Hz, 1 H) 8.07 (dd, J = 8.5, 1.8 Hz, 1 H) 8.22 (d, J = 8.6 |
| | Hz, 1 H) 8.32 (d, J = 1.8 Hz, 1 H) 9.21 (dd, J = 1.7, 0.9 Hz, 1 H) 9.23 |
| | (d, J = 0.7 Hz, 1 H) 10.59 (s, 1 H) |
| Final Cpd 37 | ¹H NMR (400 MHz, DMSO-d₆) δ ppm 1.21 (s, 3 H) 1.24 (d, J = 6.8 |
| | Hz, 6 H) 1.89-1.98 (m, 2 H) 2.18-2.26 (m, 2 H) 3.60 (spt, J = 6.7 |
| | Hz, 1 H) 3.70-3.83 (m, 1 H) 4.65 (s, 2 H) 8.04 (dd, J = 8.5, 1.8 Hz, 1 |
| | H) 8.19 (d, J = 8.6 Hz, 1 H) 8.28 (br d, J = 7.0 Hz, 1 H) 8.28 (d, J = 1.9 |
| | Hz, 1 H); 1H exchanged |
| Final Cpd 38 | ¹H NMR (400 MHz, DMSO-d₆) δ ppm 1.25 (d, J = 6.7 Hz, 6 H) 3.64 |
| | (spt, J = 6.7 Hz, 1 H) 4.97-5.14 (m, 2 H) 7.98 (dd, J = 5.9, 1.3 Hz, 1 |
| | H) 8.06 (dd, J = 8.5, 1.8 Hz, 1 H) 8.21 (d, J = 8.5 Hz, 1 H) 8.32 (d, |
| | J = 1.9 Hz, 1 H) 8.66 (d, J = 5.7 Hz, 1 H) 8.91-8.93 (m, 1 H) 11.32 (s, 1 H) |
| Final Cpd X2 | ¹H NMR (400 MHz, DMSO-d6) δ ppm 1.29 (d, J = 6.7 Hz, 6 H) 3.77 |
| | (spt, J = 6.7 Hz, 1 H) 5.10 (s, 2 H) 7.92 (br d, J = 10.0 Hz, 1 H) 8.22 |
| | (dd, J = 8.4, 1.1 Hz, 1 H) 8.35 (dd, J = 10.1, 0.6 Hz, 1 H) 8.42 (s, 1 H) |
| | 8.51 (d, J = 8.4 Hz, 1 H) 9.52 (d, J = 0.6 Hz, 1 H) 11.47 (br s, 1 H) |
| Final Cpd X3 | ¹H NMR (400 MHz, DMSO-d₆) δ ppm 1.28 (d, J = 6.4 Hz, 6 H) 3.55- |
| | 3.71 (m, 1 H) 5.11 (s, 2 H) 7.93 (br d, J = 9.9 Hz, 1 H) 8.22-8.29 |
| | (m, 1 H) 8.29-8.38 (m, 2 H) 9.52 (s, 1 H) 11.53 (br s, 1 H) |
| Final Cpd X4 | ¹H NMR (400 MHz, DMSO-d₆) δ ppm 0.86-0.93 (m, 2 H) 0.99 (dt, |
| | J = 9.7, 4.5 Hz, 2 H) 1.21 (s, 3 H) 1.87-2.00 (m, 2 H) 2.17-2.27 |
| | (m, 2 H) 2.61 (ddd, J = 13.1, 8.2, 5.0 Hz, 1 H) 3.70-3.82 (m, 1 H) |
| | 4.64 (s, 2 H) 4.95 (s, 1H) 8.22 (dd, J = 12.5, 7.8 Hz, 2 H) 8.47 (d, J = |
| | 8.3 Hz, 1 H) 8.59 (s, 1 H) |
| Final Cpd X5 | ¹H NMR (400 MHz, DMSO-d₆) δ ppm 1.25-1.32 (m, 6 H) 3.20- |
| | 3.27 (m, 3 H) 3.72-3.80 (m, 1 H) 4.91-4.97 (m, 2 H) 7.01 (d, |
| | J = 8.5 Hz, 1 H), 7.11 (d, J = 8.5 Hz, 1H), 7.43 (s, 1 H), 8.21 (d, J = 8.5 |
| | Hz, 1 H), 8.39-8.43 (m, 1H), 8.52 (dt, J = 8.4, 0.9 Hz, 1 H), 10.13- |
| | 10.18 (m, 1 H) |
| Final Cpd X6 | ¹H NMR (400 MHz, DMSO-d₆, 61° C.) δ ppm 0.95 (t, J = 7.2 Hz, 3 |
| | H) 1.29 (d, J = 6.7 Hz, 6 H) 1.56-1.74 (m, 2 H) 2.20-2.46 (m, 6 H) |
| | 3.71 (spt, J = 6.6 Hz, 2 H) 3.80-3.91 (m, 1 H) 4.54 (br s, 1 H) 4.80 |
| | (d, J = 1.5 Hz, 2 H) 7.52 (br d, J = 8.3 Hz, 1 H) 8.17 (d, J = 8.3 Hz, 1 H) |
| | 8.35 (s, 1 H) 8.51 (d, J = 8.3 Hz, 1 H) |
| Final Cpd X8 | ¹H NMR (400 MHz, CDCl₃) δ ppm 1.41 (t, J = 7.4 Hz, 3 H), 3.08 (q, |
| | J = 7.4 Hz, 2 H) 5.10 (s, 2 H) 7.02 (d, J = 9.6 Hz, 1 H) 7.70 (d, J = 9.7 |
| | Hz, 1 H) 8.04 (d, J = 8.5 Hz, 1 H) 8.12 (s, 1 H) 8.65 (d, J = 8.3 Hz, 1 |
| | H) 8.74 (s, 1 H) 9.20 (s, 1 H) 9.24 (s, 1 H) |
| Final Cpd X9 | ¹H NMR (400 MHz, DMSO-d₆) δ ppm 0.95 (t, J = 7.2 Hz, 3 H) 1.17- |
| | 1.26 (m, 7 H) 1.37-1.55 (m, 1 H) 1.59-1.74 (m, 2 H) 1.81 (brt, |
| | J = 9.7 Hz, 1 H) 1.93 (brt, J = 10.1 Hz, 1 H) 2.20-2.38 (m, 2 H) 2.55- |
| | 2.64 (m, 1 H) 2.72 (br d, J = 7.9 Hz, 1 H) 3.66-3.79 (m, 2 H) 4.72 |
| | (d, J = 2.2 Hz, 2 H) 7.95 (br d, J = 8.1 Hz, 1 H) 8.19 (d, J = 8.4 Hz, 1 H) |
| | 8.38 (s, 1 H) 8.50 (d, J = 8.4 Hz, 1 H) |
| Final Cpd X10 | ¹H NMR (400 MHz, DMSO-d₆) δ ppm 0.21-0.27 (m, 2 H) 0.37 (br |
| | d, J = 6.4 Hz, 2 H) 1.22 (br s, 1 H) 1.26 (d, J = 6.6 Hz, 6 H) 1.32-1.47 |
| | (m, 1 H) 1.56-1.60 (m, 1 H) 1.60-1.65 (m, 1 H) 1.65-1.73 (m, 1 |
| | H) 2.04 (br t, J = 9.8 Hz, 1 H) 2.17 (br t, J = 9.5 Hz, 1 H) 2.69 (br d, |
| | J = 10.6 Hz, 1 H) 2.79-2.93 (m, 1 H) 3.58-3.69 (m, 1 H) 3.69-3.76 |
| | (m, 1 H) 4.72 (s, 2 H) 7.90 (br d, J = 7.7 Hz, 1 H) 8.19 (d, J = 8.4 Hz, |
| | 1 H) 8.38 (s, 1 H) 8.50 (d, J = 8.4 Hz, 1 H) |
| Final Cpd X11 | ¹H NMR (400 MHz, DMSO-d₆) δ ppm 1.28 ppm (d, J = 6.7 Hz, 6 H) |
| | 3.70-3.81 (m, 1 H) 5.18 (s, 2 H) 8.21 (d, J = 8.4 Hz, 1 H) 8.41 (s, 1 |
| | H) 8.46 (s, 1 H) 8.52 (d, J = 8.4 Hz, 1 H) 11.09 (s, 1 H) |
| Final Cpd X13 | ¹H NMR (400 MHz, DMSO-d₆, 150° C.) δ ppm 1.34 (d, J = 6.8 Hz, 6 |
| | H) 3.69 (spt, J = 6.7 Hz, 1 H) 5.09 (s, 2 H) 7.82-7.89 (m, 1 H) 7.90- |
| | 7.99 (m, 1 H) 8.14 (dd, J = 8.4, 1.1 Hz, 1 H) 8.21 (s, 1 H) 8.32 (s, 1 |
| | H) 8.55 (d, J = 8.4 Hz, 1 H) 10.17 (br s, 1 H) 11.92-12.73 (m, 1 H) |
| Final Cpd X14 | ¹H NMR (400 MHz, DMSO-d₆) δ ppm 1.28 (d, J = 6.6 Hz, 6 H) 3.76 |
| | (dt, J = 13.4, 6.6 Hz, 1 H) 5.08 (s, 2 H) 8.21 (br d, J = 8.6 Hz, 1 H) |
| | 8.41 (s, 1 H) 8.51 (d, J = 8.1 Hz, 1 H) 9.19 (s, 1 H) 9.35 (s, 1 H) 9.41 |
| | (s, 1 H) 11.15 (br s, 1 H) |
| Final Cpd X15 | ¹H NMR (400 MHz, DMSO-d₆, 101° C.) δ ppm 0.18-0.24 (m, 2 H) |
| | 0.33-0.40 (m, 2 H) 1.31 (d, J = 6.8 Hz, 6 H) 1.56-1.60 (m, 1 H) |
| | 1.61-1.66 (m, 2 H) 2.42-2.63 (m, 4 H) 3.66-3.75 (m, 2 H) 3.77- |
| | 3.87 (m, 1 H) 4.38 (br s, 1 H) 4.80 (d, J = 2.2 Hz, 2 H) 7.24 (br d, |
| | J = 7.3 Hz, 1 H) 8.15 (dd, J = 8.4, 1.1 Hz, 1 H) 8.33 (s, 1 H) 8.53 (d, |
| | J = 8.4 Hz, 1 H) |
| Final Cpd X16 | ¹H NMR (400 MHz, DMSO-d₆) δ ppm 1.29 (d, J = 6.5 Hz, 6 H) 3.76 |
| | (spt, J = 6.7 Hz, 1 H) 5.07 (s, 2 H) 7.73 (d, J = 1.2 Hz, 1 H) 7.83 (br d, |
| | J = 9.4 Hz, 1 H) 8.11 (d, J = 10.6 Hz, 1 H) 8.16 (s, 1 H) 8.21 (dd, J = |
| | 8.6, 1.2 Hz, 1 H) 8.41 (s, 1 H) 8.52 (d, J = 8.2 Hz, 1 H) 11.25 (br s, 1 H) |
| Final Cpd X17 | ¹H NMR (500 MHz, DMSO-d₆) δ ppm 1.21 (s, 3 H) 1.23-1.30 (m, |
| | 6 H) 1.90-1.98 (m, 2 H) 2.19-2.26 (m, 2 H) 3.55-3.64 (m, 1 H) |
| | 3.73-3.83 (m, 1 H) 4.68 (s, 2 H) 4.97 (s, 1 H) 8.22 (dd, J = 8.3, 6.6 |
| | Hz, 1 H) 8.26-8.36 (m, 1 H) |
| Final Cpd X18 | ¹H NMR (400 MHz, DMSO-d₆) δ ppm 1.29 (d, J = 6.7 Hz, 6 H) 3.78 |
| | (spt, J = 6.7 Hz, 1 H) 5.14 (s, 2 H) 8.22 (dd, J = 8.4, 1.1 Hz, 1 H) 8.38 |
| | (d. J = 9.9 Hz, 1 H) 8.43 (s, 1 H) 8.52 (d, J = 8.3 Hz, 1 H) 8.74 (d, |
| | J = 9.8 Hz, 1 H) 11.92 (br s, 1 H) |
| Final Cpd X19 | ¹H NMR (400 MHz, DMSO-d₆) δ ppm 0.88-1.04 (m, 7 H) 1.56- |
| | 1.69 (m, 2 H) 2.18-2.45 (m, 6 H) 2.52-2.67 (m, 1 H) 3.68-3.85 |
| | (m, 2 H) 4.69-4.79 (m, 3 H) 7.75 (d, J = 8.4 Hz, 1 H) 8.21 (d, J = 8.5 |
| | Hz, 1 H) 8.47 (d, J = 8.3 Hz, 1 H) 8.59 (s, 1 H) |
| Final Cpd X20 | ¹H NMR (400 MHz, DMSO-d₆) δ ppm 1.27-1.32 (m, 6 H) 3.77 (dt, |
| | J = 13.4, 6.7 Hz, 1 H) 5.00 (s, 2 H) 6.74 (dd, J = 9.7, 1.7 Hz, 1 H) 7.31 |
| | (s, 1 H) 7.55 (d, J = 9.6 Hz, 1 H) 8.21 (dd, J = 8.4, 1.6 Hz, 1 H) 8.34 |
| | (s, 1 H) 8.42 (s, 1 H) 8.52 (d, J = 8.1 Hz, 1 H) 8.98 (s, 1 H) 10.29- |
| | 10.35 (m, 1 H) |
| Final Cpd X21 | ¹H NMR (400 MHz, DMSO-d₆) ppm 1.28 (d, J = 6.6 Hz, 6 H) 3.77 |
| | (spt, J = 6.6 Hz, 1 H) 5.13 (s, 2 H) 8.22 (dd, J = 8.4, 1.1 Hz, 1 H) 8.42 |
| | (s, 1 H) 8.51 (d, J = 8.4 Hz, 1 H) 9.42 (d, J = 2.0 Hz, 1 H) 9.91 (d, |
| | J = 2.0 Hz, 1 H) 11.77 (br s, 1 H) |
| Final Cpd X22 | ¹H NMR (400 MHz, DMSO-d₆) δ ppm 1.28 (d, J = 6.5 Hz, 6 H) 3.76 |
| | (spt, J = 6.8 Hz, 1 H) 4.98 (s, 2 H) 6.71 (s, 1 H) 6.75 (s, 1 H) 8.21 |
| | (dd, J = 8.2, 1.2 Hz, 1 H) 8.41 (s, 1 H) 8.51 (d, J = 8.6 Hz, 1 H) 10.78 |
| | (br s, 1 H) 11.23 (br s, 1 H) |
| Final Cpd X23 | ¹H NMR (400 MHz, DMSO-d₆) δ ppm 0.98 (m, 4 H), 2.65 (m, 1 H), |
| | 4.98 (s, 2H), 7.31 (dd, J = 9.8, 1.9 Hz, 1 H), 7.79 (d, J = 9.7 Hz, 1 H), |
| | 8.23 (dd, J = 8.4, 1.3 Hz, 1 H), 8.50 (d, J = 8.4 Hz, 1 H), 8.63 (s, 1H), |
| | 9.19 (s, 1 H), 9.23 (d, J = 0.7 Hz, 1 H), 10.54 (s, 1H). |
| Final Cpd X24 | ¹H NMR (400 MHz, DMSO-d₆) δ ppm 0.89 (t, J = 7.4 Hz, 3 H), 1.26 |
| | (d, J = 6.8 Hz, 3 H), 1.54-1.71 (m, 1 H), 1.83 (dquin, J = 13.8, 7.1, |
| | 7.1, 7.1, 7.1 Hz, 1 H), 3.35-3.63 (m, 1 H), 4.96-5.12 (m, 2 H), |
| | 7.32 (dd, J = 9.7, 2.0 Hz, 1 H), 7.80 (d, J = 9.7 Hz, 1 H), 8.22 (d, J = 8.4 |
| | Hz, 1 H), 8.37-8.55 (m, 2 H), 9.19-9.26 (m, 2 H), 10.61 (br s, 1 H) |
| Final Cpd X25 | ¹H NMR (400 MHz, DMSO-d₆) δ ppm 1.29 (d, J = 6.7 Hz, 6 H) 3.71- |
| | 3.83 (m, 4 H) 5.00 (s, 2 H) 7.20 (dd, J = 8.7, 2.0 Hz, 1 H) 7.58 (d, |
| | J = 8.7 Hz, 1 H) 8.03 (d, J = 1.7 Hz, 1 H) 8.10 (s, 1 H) 8.22 (dd, J = 8.4, |
| | 1.3 Hz, 1 H) 8.42 (s, 1 H) 8.52 (d, J = 8.3 Hz, 1 H) 10.38 (s, 1 H) |
| | ¹⁹F NMR (376 MHz, DMSO-d₆) δ ppm −61.28 (s, 3 F) |
| Final Cpd X26 | ¹H NMR (400 MHz, DMSO-d₆) δ ppm 1.28 (d, J = 6.0 Hz, 6 H) 3.56- |
| | 3.70 (m, 1 H) 5.02 (s, 2 H) 7.31 (dd, J = 9.7, 1.9 Hz, 1 H) 7.80 (d, |
| | J = 9.7 Hz, 1 H) 8.21-8.28 (m, 1 H) 8.30-8.37 (m, 1 H) 9.17-9.22 |
| | (m, 1 H) 9.23 (d, J = 0.7 Hz, 1 H) 10.59 (s, 1 H) |
| Final Cpd X27 | ¹H NMR (400 MHz, DMSO-d₆) δ ppm 1.29 (d, J = 6.6 Hz, 6 H) 2.29 |
| | (s, 3 H) 3.77 (spt, J = 6.6 Hz, 1 H) 4.99 (s, 2 H) 7.12 (dd, J = 9.6, 2.1 |
| | Hz, 1 H) 7.42 (d, J = 9.5 Hz, 1 H) 7.67 (s, 1 H) 8.21 (dd, J = 8.5, 1.2 |
| | Hz, 1 H) 8.42 (s, 1 H) 8.52 (d, J = 8.4 Hz, 1 H) 9.02 (d, J = 1.1 Hz, 1 |
| | H) 10.34 (s, 1 H) |
| | ¹⁹F NMR (377 MHz, DMSO-d₆) δ ppm −61.29 (s, 3 F) |
| Final Cpd X28 | 1H NMR (400 MHz, DMSO-d₆) δ ppm 1.29 (d, J = 6.9 Hz, 6 H) 2.54 |
| | (s, 3 H) 3.77 (spt, J = 6.7 Hz, 1 H) 5.01 (s, 2 H) 7.10 (t, J = 1.4 Hz, 1 |
| | H) 8.22 (dd, J = 8.4, 1.4 Hz, 1 H) 8.42 (s, 1 H) 8.52 (d, J = 8.2 Hz, 1 |
| | H) 9.04 (s, 1 H) 9.21 (s, 1 H) 10.49 (br s, 1 H) |
| | ¹⁹F NMR (377 MHz, DMSO-d₆) δ ppm −61.30 (s, 3 F) |
| Final Cpd X29 | ¹H NMR (400 MHz, CDCl₃) δ ppm 1.29 (s, 3 H) 1.38 (d, J = 6.8 Hz, |
| | 6 H) 1.87-1.94 (m, 2 H) 2.35-2.43 (m, 2 H) 3.12 (s, 3 H) 3.52 |
| | (spt, J = 6.8 Hz, 1 H) 4.14 (sxt, J = 8.0 Hz, 1 H) 4.86 (s, 2 H) 6.28 (br |
| | d, J = 7.0 Hz, 1 H) 7.99 (dd, J = 8.4, 1.1 Hz, 1 H) 8.14 (s, 1 H) 8.63 (d, |
| | J = 8.4 Hz, 1 H) |
| | ¹⁹F NMR (377 MHz, CDCl₃) δ ppm −63.00 (s, 3 F) |
| Final Cpd X30 | ¹H NMR (400 MHz, CDCl₃) δ ppm 1.41 (t, J = 7.4 Hz, 3H) 3.08 (q, |
| | J = 7.4 Hz, 2H) 5.11 (s, 2H) 8.04 (dd, J = 9.6, 6.1 Hz, 2H) 8.13 (s, 1H) |
| | 8.66 (d, J = 8.3 Hz, 1H) 9.02 (d, J = 5.9 Hz, 1H) 9.14 (d, J = 2.1 Hz, |
| | 1H) 9.44 (s, 1H) |
| Final Cpd X31 | ¹H NMR (400 MHz, DMSO-d₆) δ ppm 1.29 (d, J = 6.5 Hz, 6 H) 3.77 |
| | (spt, J = 6.8 Hz, 1 H) 5.01 (s, 2 H) 7.18 (dd, J = 9.8, 2.0 Hz, 1 H) 7.52 |
| | (d, J = 1.2 Hz, 1 H) 7.56 (d, J = 9.8 Hz, 1 H) 7.91-7.99 (m, 1 H) 8.21 |
| | (dd, J = 8.6, 1.2 Hz, 1 H) 8.42 (s, 1 H) 8.52 (d, J = 8.2 Hz, 1 H) 9.14 |
| | (dd, J = 2.0, 0.8 Hz, 1 H) 10.39 (s, 1 H) |
| | ¹⁹F NMR (377 MHz, DMSO-d₆) δ ppm −61.30 (s, 3 F) |
| Final Cpd X32 | ¹H NMR (400 MHz, DMSO-d₆) δ ppm 5.12 (s, 2 H) 7.35 (br t, |
| | J = 52.5 Hz, 1 H) 7.33 (br d, J = 9.9 Hz, 2 H) 7.81 (d, J = 9.7 Hz, 1 H) |
| | 8.35 (br d, J = 8.5 Hz, 1 H) 8.38 (s, 1 H) 8.59 (d, J = 8.3 Hz, 1 H) 9.20 |
| | (s, 1 H) 9.24 (s, 1 H) 10.71 (br s, 1 H) |
| Final Cpd X33 | ¹H NMR (400 MHz, DMSO-d₆) δ ppm 1.24-1.31 (m, 6 H) 3.60 (t, |
| | J = 0.5 Hz, 3 H) 3.70-3.81 (m, 1 H) 4.94-5.02 (m, 2 H) 6.98 (d, |
| | J = 9.9 Hz, 1 H) 7.89-7.98 (m, 1 H) 8.22 (d, J = 8.1 Hz, 1 H) 8.39- |
| | 8.43 (m, 1 H) 8.51 (dt, J = 8.4, 0.9 Hz, 1H) 10.98 (br s, 1 H) |
| Final Cpd X34 | ¹H NMR (400 MHz, DMSO-d₆) δ ppm 0.91-0.96 (m, 2 H) 1.00- |
| | 1.04 (m, 2 H) 2.59-2.71 (m, 1 H) 5.05 (s, 2 H) 7.91 (d, J = 10.0 Hz, |
| | 1 H) 8.24 (dd, J = 8.4, 1.3 Hz, 1 H) 8.35 (d, J = 10.1 Hz, 1 H) 8.49 (d, |
| | J = 8.3 Hz, 1 H) 8.63 (s, 1 H) 9.53 (d, J = 0.5 Hz, 1 H) 11.46 (s, 1 H) |
| Final Cpd X35 | ¹H NMR (400 MHz, CDCl₃) δ ppm 1.39 (d, J = 6.8 Hz, 6 H) 2.25- |
| | 2.36 (m, 2 H) 2.89-3.02 (m, 2 H) 3.53 (spt, J = 6.8 Hz, 1 H) 3.62 (s, |
| | 1 H) 3.98-4.11 (m, 1 H) 4.88 (s, 2 H) 6.70 (br d, J = 6.6 Hz, 1 H) |
| | 7.98 (dd, J = 8.4, 1.3 Hz, 1 H) 8.15 (s, 1 H) 8.61 (d, J = 8.4 Hz, 1 H) |
| | 8.69-8.74 (m, 1 H) |
| | ¹⁹F NMR (376 MHz, CDCl₃) δ ppm −84.42 (s, 3 F), −63.02 (s, 3 F) |
| Final Cpd X36 | ¹H NMR (400 MHz, DMSO-d₆) δ ppm 8.55 (d, J = 8.3 Hz, 1H) 8.39 |
| | (d, J = 7.0 Hz, 1H) 8.34-8.29 (m, 2H) 7.28 (t, J = 52.4 Hz, 1H) 4.99 |
| | (s, 1H) 4.77 (s, 2H) 3.83-3.70 (m, 1H) 2.27-2.19 (m, 2H) 1.94 (td, |
| | J = 8.9, 2.2 Hz, 2H) 1.21 (s, 3H) |
| Final Cpd X37 | ¹H NMR (400 MHz, DMSO-d₆, 81° C.) δ ppm 1.09 (br t, J = 6.8 Hz, 3 |
| | H) 1.34 (d, J = 6.7 Hz, 6 H) 1.64-1.87 (m, 1 H) 1.96-2.12 (m, 1 H) |
| | 2.14-2.41 (m, 1 H) 2.57-2.75 (m, 3 H) 2.84-3.01 (m, 2 H) 3.74 |
| | (spt, J = 6.8 Hz, 1 H) 4.09-4.25 (m, 1 H) 4.80 (d, J = 2.6 Hz, 2 H) |
| | 4.87-5.12 (m, 1 H) 7.94 (br d, J = 3.7 Hz, 1 H) 8.20 (dd, J = 8.4, 1.3 |
| | Hz, 1 H) 8.38 (s, 1 H) 8.56 (d, J = 8.4 Hz, 1 H) |
| | ¹⁹F NMR (377 MHz, DMSO-d₆) δ ppm −[182.95-180.16] (m, |
| | 1 F), −61.31 (s, 3 F) |
| Final Cpd X38 | ¹H NMR (400 MHz, DMSO-d₆) δ ppm 1.26-1.33 (m, 6 H) 3.71- |
| | 3.84 (m, 1 H) 5.01-5.09 (m, 2 H) 7.64 (dd, J = 9.5, 2.0 Hz, 1 H) 7.87 |
| | (dd, J = 9.6, 0.8 Hz, 1 H) 8.22 (d, J = 8.5 Hz, 1 H) 8.41-8.49 (m, 2 H) |
| | 8.53 (dt, J = 8.4, 0.9 Hz, 1 H) 9.36-9.41 (m, 1 H) 10.68-10.78 (m, 1 H) |
| Final Cpd X39 | ¹H NMR (400 MHz, CDCl₃) δ ppm 1.36-1.43 (m, 6 H) 3.55 (quin, |
| | J = 6.8 Hz, 1 H) 5.04-5.15 (m, 2 H) 6.21 (s, 1 H) 6.62 (t, J = 54.9 Hz, |
| | 1 H) 8.02 (d, J = 7.9 Hz, 1 H) 8.13-8.18 (m, 1 H) 8.63 (d, J = 8.3 Hz, |
| | 1 H) 9.67 (br s, 1 H) |
| Final Cpd X40 | ¹H NMR (400 MHz, DMSO-d₆) δ ppm 1.24-1.31 (m, 6 H), 2.15- |
| | 2.22 (m, 6 H), 2.52-2.63 (m, 2 H), 3.76 (quin, J = 6.7 Hz, 1 H), 4.09 |
| | (t, J = 6.6 Hz, 2 H), 4.96-5.01 (m, 2 H), 6.96 (d, J = 9.9 Hz, 1 H), |
| | 7.91 (br d, J = 10.0 Hz, 1 H), 8.21 (dd, J = 8.4, 1.5 Hz, 1 H), 8.41 (s, 1 |
| | H), 8.50 (d, J = 8.3 Hz, 1 H), 10.94-11.00 (m, 1 H) |
| Final Cpd X41 | ¹H NMR (400 MHz, CDCl₃) δ ppm 1.38 (d, J = 6.7 Hz, 6 H) 1.90- |
| | 2.01 (m, 1 H) 2.09-2.20 (m, 1 H) 2.28 (s, 3 H) 2.91-3.10 (m, 2 H) |
| | 3.51 (spt, J = 6.8 Hz, 1 H) 3.77 (dd, J = 12.4, 4.7 Hz, 1 H) 3.97 (dd, |
| | J = 12.5, 4.7 Hz, 1 H) 4.52-4.68 (m, 1 H) 4.92-5.04 (m, 2 H) 7.63 |
| | (br d, J = 7.4 Hz, 1 H) 7.97 (dd, J = 8.4, 1.1 Hz, 1 H) 8.11 (s, 1 H) 8.58 |
| | (d, J = 8.4 Hz, 1 H) |
| | ¹⁹F NMR (376 MHz, CDCl₃) δ ppm −62.97 (s, 3 F) |
| Final Cpd X42 | ¹H NMR (400 MHz, CDCl₃) δ ppm 1.38 (d, J = 6.8 Hz, 6 H) 1.58- |
| | 1.75 (m, 8 or 9 H) 3.53 (spt, J = 6.7 Hz, 1 H) 3.82-3.97 (m, 2 H) |
| | 4.88 (s, 2 H) 5.95-6.17 (m, 1 H) 7.99 (dd, J = 8.3, 1.2 Hz, 1 H) 8.14 |
| | (s, 1 H) 8.63 (d, J = 8.4 Hz, 1 H) |
| | ¹⁹F NMR (377 MHz, CDCl₃) δ ppm −63.00 (s, 3 F) |
| Final Cpd X44 | ¹H NMR (400 MHz, DMSO-d₆) δ ppm 1.16-1.29 (m, 10 H) 1.70- |
| | 1.86 (m, 4 H) 3.32-3.42 (m, 1 H) 3.44-3.58 (m, 1 H) 3.72 (spt, |
| | J = 6.7 Hz, 1 H) 4.50 (d, J = 4.5 Hz, 1 H) 4.69 (s, 2 H) 7.91 (d, J = 7.8 |
| | Hz, 1 H) 8.18 (dd, J = 8.2, 1.2 Hz, 1 H) 8.38 (s, 1 H) 8.49 (d, J = 8.2 |
| | Hz, 1 H) |
| | ¹⁹F NMR (377 MHz, DMSO-d₆) δ ppm −61.30 (s, 3 F) |
| Final Cpd X45 | ¹H NMR (400 MHz, DMSO-d₆) δ ppm 0.19-0.31 ppm (m, 2 H) |
| | 0.31-0.41 (m, 2 H) 0.87-0.96 (m, 2 H) 0.96-1.04 (m, 2 H) 1.51- |
| | 1.65 (m, 3 H) 2.40-2.57 (m, 4 H) 2.57-2.65 (m, 1 H) 3.66-3.77 |
| | (m, 2 H) 4.68-4.79 (m, 2 H) 4.81 (br d, J = 3.6 Hz, 1 H) 7.78 (br d, |
| | J = 7.7 Hz, 1 H) 8.21 (d, J = 8.5 Hz, 1H) 8.47 (d, J = 8.3 Hz, 1 H) 8.57- |
| | 8.62 (m, 1 H) |
| Final Cpd X46 | ¹H NMR (400 MHz, DMSO-d₆) δ ppm 1.04-1.13 (m, 3 H), 1.21- |
| | 1.28 (m, 6 H), 1.28-1.33 (m, 2 H), 1.43-1.62 (m, 6 H), 2.05-2.10 |
| | (m, 1 H), 3.45-3.56 (m, 1 H), 3.72 (quin, J = 6.7 Hz, 1 H), 4.00 (s, 1 |
| | H), 4.69 (s, 2 H), 7.93 (br d, J = 7.8 Hz, 1 H), 8.18 (d, J = 8.4 Hz, 1 H), |
| | 8.37 (s, 1 H), 8.49 (d, J = 8.3 Hz, 1 H) |
| Final Cpd X47 | ¹H NMR (400 MHz, CDCl₃) δ ppm 1.44 (d, J = 6.8 Hz, 6 H) 3.57 |
| | (quin, J = 6.8 Hz, 1 H) 3.80 (s, 3 H) 5.13 (s, 2 H) 7.94 (br s, 1 H) 7.98- |
| | 8.07 (m, 2 H) 8.16 (s, 2 H) 8.68 (d, J = 8.4 Hz, 1 H) 8.76 (br s, 1 H) |
| Final Cpd X48 | ¹H NMR (400 MHz, CDCl₃) δ ppm 1.42 (d, J = 6.8 Hz, 6 H) 2.53 (br |
| | s, 3 H) 3.56 (dt, J = 13.6, 6.7 Hz, 1 H) 5.09 (s, 2 H) 6.59 (br d, J = 6.4 |
| | Hz, 1 H) 7.01-7.24 (m, 1 H) 7.43 (br d, J = 7.3 Hz, 1 H) 7.76 (br s, 1 |
| | H) 8.01 (d, J = 8.1 Hz, 1 H) 8.16 (s, 1 H) 8.65 (br d, J = 8.1 Hz, 1 H) |
| | 9.11 (br s, 1 H) |
| Final Cpd X49 | ¹H NMR (400 MHz, DMSO-d₆) δ ppm 1.29 (d, J = 6.6 Hz, 6 H) 3.77 |
| | (dt, J = 13.4, 6.6 Hz, 1 H) 4.20 (s, 3 H) 5.04 (s, 2 H) 7.30-7.41 (m, 1 |
| | H) 7.98 (d, J = 8.8 Hz, 1 H) 8.21 (br d, J = 8.4 Hz, 1 H) 8.28 (s, 1 H) |
| | 8.42 (s, 1 H) 8.52 (br d, J = 8.4 Hz, 1 H) 10.69 (s, 1 H) |
| | ¹⁹F NMR (377 MHz, DMSO-d₆) δ ppm −61.30 (s, 3 F) |
| Final Cpd X50 | ¹H NMR (400 MHz, CDCl₃) δ ppm 5.10 (s, 2 H) 6.67 (t, J = 53.2 Hz, |
| | 1 H) 8.09 (d, J = 7.0 Hz, 2 H) 8.46 (s, 2 H) 8.64 (s, 1 H) 8.66 (d, |
| | J = 2.7 Hz, 1 H) 8.90 (s, 1 H) |
| Final Cpd X51 | ¹H NMR (400 MHz, DMSO-d₆) δ ppm 1.27 (d, J = 6.7 Hz, 6 H) 2.07 |
| | (d, J = 1.2 Hz, 3 H) 3.60 (s, 3 H) 3.74 (spt, J = 6.8 Hz, 1 H) 4.96 (s, 2 |
| | H) 7.85 (br s, 1 H) 8.19 (dd, J = 8.4, 1.2 Hz, 1 H) 8.39 (s, 1 H) 8.49 |
| | (d, J = 8.3 Hz, 1 H) 10.83 (s, 1 H) |
| | ¹⁹F NMR (376 MHz, DMSO-d₆) δ ppm −61.34 (s, 3 F) |
| Final Cpd X52 | ¹H NMR (400 MHz, DMSO-d₆) δ ppm 1.29 (d, J = 6.9 Hz, 6 H) 3.77 |
| | (spt, J = 6.7 Hz, 1 H) 5.01 (s, 2 H) 7.68 (d, J = 1.6 Hz, 1 H) 8.22 (dd, |
| | J = 8.6, 1.2 Hz, 1 H) 8.43 (s, 1 H) 8.52 (d, J = 8.2 Hz, 1 H) 9.20 (d, |
| | J = 1.6 Hz, 1 H) 9.37 (s, 1 H) 10.58 (br s, 1 H) |
| | ¹⁹F NMR (377 MHz, DMSO-d₆) δ ppm −61.31 (s, 3 F) |
| Final Cpd X53 | ¹H NMR (400 MHz, DMSO-d₆) δ ppm 1.28 (d, J = 6.5 Hz, 6 H) 2.37 |
| | (s, 3 H) 3.76 (spt, J = 6.7 Hz, 1 H) 5.05 (s, 2 H) 7.76 (br d, J = 9.8 Hz, |
| | 1 H) 7.92 (s, 1 H) 7.98 (s, 1 H) 8.21 (dd, J = 8.4, 1.4 Hz, 1 H) 8.41 (s, |
| | 1 H) 8.51 (d, J = 8.6 Hz, 1 H) 11.18 (br s, 1 H) |
| | ¹⁹F NMR (376 MHz, DMSO-d₆) δ ppm −61.32 (s, 3 F) |
| Final Cpd X54 | ¹H NMR (400 MHz, DMSO-d₆) δ ppm 1.29 (d, J = 6.8 Hz, 6 H) 3.77 |
| | (spt, J = 6.7 Hz, 1 H) 5.01 (s, 2 H) 6.99 (dd, J = 7.3, 2.1 Hz, 1 H) 7.46 |
| | (d, J = 1.1 Hz, 1 H) 7.80-7.86 (m, 1 H) 7.87-7.94 (m, 1 H) 8.22 |
| | (dd, J = 8.4, 1.3 Hz, 1 H) 8.42 (s, 1 H) 8.47 (dd, J = 7.3, 0.7 Hz, 1 H) |
| | 8.52 (d, J = 8.3 Hz, 1 H) 10.55 (s, 1 H) |
| | ¹⁹F NMR (377 MHz, DMSO-d₆) δ ppm −61.31 (s, 3 F) |
| Final Cpd X55 | ¹H NMR (400 MHz, CDCl₃) δ ppm 1.37 (d, J = 6.9 Hz, 6 H) 1.95- |
| | 2.07 (m, 1 H) 2.16-2.28 (m, 1 H) 2.94-3.10 (m, 2 H) 3.50 (spt, |
| | J = 6.8 Hz, 1 H) 4.04 (dd, J = 12.6, 4.1 Hz, 1 H) 4.14 (dd, J = 12.6, 4.5 |
| | Hz, 1 H) 4.51-4.67 (m, 1 H) 4.93 (d, J = 15.1 Hz, 1 H) 5.01 (d, |
| | J = 15.1 Hz, 1 H) 7.68 (br d, J = 7.3 Hz, 1 H) 7.87 (s, 1 H) 7.96 (dd, |
| | J = 8.2, 1.2 Hz, 1 H) 8.11 (s, 1 H) 8.57 (d, J = 8.2 Hz, 1 H) |
| | ¹⁹F NMR (377 MHz, CDCl₃) δ ppm −62.98 (s, 3 F) |
| Final Cpd X56 | ¹H NMR (400 MHz, CDCl₃) δ ppm 1.31-1.41 (m, 7 H) 1.68 (tt, |
| | J = 10.9, 8.9 Hz, 1 H) 2.01-2.10 (m, 1 H) 2.10-2.20 (m, 1 H) 3.37 |
| | (d, J = 2.7 Hz, 1 H) 3.53 (spt, J = 6.8 Hz, 1 H) 3.80-3.92 (m, 1 H) |
| | 3.96-4.06 (m, 1 H) 4.88 (d, J = 1.5 Hz, 2 H) 6.53 (br s, 1 H) 7.99 |
| | (dd, J = 8.4, 1.2 Hz, 1 H) 8.14 (s, 1 H) 8.61 (d, J = 8.4 Hz, 1 H) |
| | ¹⁹F NMR (376 MHz, CDCl₃) δ ppm −63.00 (s, 3 F) |
| Final Cpd X57 | ¹H NMR (400 MHz, DMSO-d₆) δ ppm 1.25-1.33 (m, 6 H), 2.41- |
| | 2.47 (m, 3 H), 3.77 (quin, J = 6.7 Hz, 1 H), 4.99-5.05 (m, 2 H), 7.57 |
| | (dd, J = 9.5, 2.0 Hz, 1 H), 7.72 (dd, J = 9.5, 0.9 Hz, 1 H), 8.22 (dd, |
| | J = 8.4, 1.6 Hz, 1 H), 8.41-8.43 (m, 1 H), 8.52 (d, J = 8.3 Hz, 1 H), |
| | 9.26 (dd, J = 2.1, 0.9 Hz, 1 H), 10.63-10.67 (m, 1 H) |
| Final Cpd X58 | ¹H NMR (400 MHz, DMSO-d₆) δ ppm 2.63 (s, 3 H) 5.18 (s, 2 H) |
| | 7.33 (dd, J = 9.8, 1.8 Hz, 1 H) 7.81 (d, J = 9.8 Hz, 1 H) 8.26 (dd, |
| | J = 8.5, 1.3 Hz, 1 H) 8.52 (d, J = 8.4 Hz, 1 H) 9.15 (s, 1 H) 9.20 (s, 1 |
| | H) 9.24 (s, 1 H) 10.72 (s, 1H) |
| Final Cpd X59 | ¹H NMR (400 MHz, DMSO-d₆) δ ppm 1.52 (d, J = 6.9 Hz, 3 H) 4.94- |
| | 5.05 (m, 1 H) 5.08 (s, 2 H) 7.31 (dd, J = 9.8, 1.8 Hz, 1 H) 7.80 (d, |
| | J = 9.8 Hz, 1 H) 8.25 (t, J = 8.3 Hz, 1 H) 8.53 (d, J = 8.3 Hz, 1 H) 8.68 |
| | (s, 1 H) 9.22 (s, 1 H) 9.24 (s, 1 H) 10.64 (s, 1 H) |
| Final Cpd X60 | ¹H NMR (400 MHz, CDCl₃) δ ppm 0.97-1.14 (m, 1 H) 1.38 (d, |
| | J = 6.8 Hz, 6 H) 1.51-1.96 (m, 10 H) 2.00-2.18 (m, 2 H) 2.97-3.11 |
| | (m, 2 H) 3.52 (spt, J = 6.8 Hz, 1 H) 3.77 (ddt, J = 15.8, 8.9, 4.3, 4.3 |
| | Hz, 1 H) 4.79-4.93 (m, 2 H) 5.92 (br d, J = 6.8 Hz, 1 H) 7.98 (dd, |
| | J = 8.4, 1.3 Hz, 1 H) 8.13 (s, 1 H) 8.62 (d, J = 8.4 Hz, 1 H) |
| | ¹⁹F NMR (377 MHz, CDCl₃) δ ppm −62.98 (s, 3 F) |
| Final Cpd X61 | ¹H NMR (400 MHz, DMSO-d₆) δ ppm 5.04 (d, J = 16.0 Hz, 1 H) |
| | 5.10 (d, J = 16.0 Hz, 1 H) 5.62-5.72 (m, 1 H) 7.31 (dd, J = 9.8, 1.9 |
| | Hz, 1 H) 7.69 (br s, 1 H) 7.80 (d, J = 9.8 Hz, 1 H) 8.26 (dd, J = 8.4, 1.3 |
| | Hz, 1 H) 8.54 (d, J = 8.4 Hz, 1 H) 8.74 (s, 1 H) 9.20 (s, 1 H) 9.23 (s, 1 |
| | H) 10.66 (br s, 1 H) |
| Final Cpd X62 | ¹H NMR (400 MHz, DMSO-d₆) δ ppm 1.22-1.34 (m, 6 H) 3.20- |
| | 3.27 (m, 3 H) 3.64-3.71 (m, 2 H) 3.76 (dt, J = 13.3, 6.7 Hz, 1 H) |
| | 4.12-4.22 (m, 2 H) 4.93-5.05 (m, 2 H) 6.98 (d, J = 9.9 Hz, 1 H) |
| | 7.86-7.99 (m, 1 H) 8.21 (br d, J = 8.3 Hz, 1 H) 8.41 (s, 1 H) 8.50 (br |
| | d, J = 8.3 Hz, 1 H) 10.93-11.05 (m, 1 H) |
| Final Cpd X63 | ¹H NMR (400 MHz, DMSO-d₆) δ ppm 1.25-1.32 (m, 6 H) 3.77 |
| | (quin, J = 6.7 Hz, 1 H) 5.08-5.14 (m, 2 H) 8.04 (d, J = 10.1 Hz, 1 H) |
| | 8.22 (dd, J = 8.4, 1.6 Hz, 1 H) 8.39 (d, J = 10.1 Hz, 1 H) 8.43 (t, J = 1.9 |
| | Hz, 1 H) 8.51 (d, J = 8.3 Hz, 1 H) 11.69-11.81 (m, 1 H) |
| Final Cpd X64 | ¹H NMR (400 MHz, DMSO-d₆) δ ppm 1.27-1.32 (m, 6 H) 3.72- |
| | 3.82 (m, 1 H) 4.99-5.08 (m, 2 H) 7.38 (dd, J = 9.7, 2.0 Hz, 1 H) 7.70 |
| | (dt, J = 9.7, 0.9 Hz, 1 H) 8.22 (d, J = 8.5 Hz, 1 H) 8.41-8.60 (m, 3 H) |
| | 9.24-9.28 (m, 1 H) 10.54-10.62 (m, 1 H) |
| Final Cpd X66 | ¹H NMR (400 MHz, DMSO-d₆) δ ppm 1.53 (d, J = 6.5 Hz, 3 H) 5.00 |
| | (d, J = 16.0 Hz, 1 H) 5.06 (d, J = 16.0 Hz, 1 H) 5.17 (p, J = 6.4 Hz, 1 H) |
| | 5.91 (d, J = 5.4 Hz, 1 H) 7.32 (dd, J = 9.8, 1.8 Hz, 1 H) 7.80 (d, J = 9.8 |
| | Hz, 1 H) 8.21 (dd, J = 8.4, 0.9 Hz, 1 H) 8.51 (d, J = 8.3 Hz, 1 H) 8.75 |
| | (s, 1 H) 9.19 (s, 1 H) 9.23 (s, 1 H) 10.64 (s, 1H) |
| Final Cpd X67 | ¹H NMR (400 MHz, DMSO-d₆) δ ppm 1.26 (d, J = 6.5 Hz, 6 H) 1.70- |
| | 1.80 (m, 2 H) 2.44-2.49 (m, 2 H) 3.64-3.82 (m, 3 H) 4.68 (s, 2 |
| | H) 5.07 (d, J = 5.3 Hz, 1 H) 8.19 (dd, J = 8.4, 1.4 Hz, 1 H) 8.24 (d, |
| | J = 7.3 Hz, 1 H) 8.38 (s, 1 H) 8.49 (d, J = 8.2 Hz, 1 H) |
| | ¹⁹F NMR (377 MHz, DMSO-d₆) δ ppm −61.30 (s, 3 F) |
| Final Cpd X68 | ¹H NMR (400 MHz, CDCl₃) δ ppm 1.38 (d, J = 6.6 Hz, 6 H) 1.92 |
| | (dtd, J = 13.6, 8.1, 2.8 Hz, 1 H) 2.14-2.24 (m, 1 H) 2.30 (s, 3 H) |
| | 2.98 (dd, J = 7.9, 5.7 Hz, 2 H) 3.50 (spt, J = 6.8 Hz, 1 H) 3.81 (dd, |
| | J = 12.5, 4.2 Hz, 1 H) 3.94 (dd, J = 12.5, 4.8 Hz, 1 H) 4.59-4.69 (m, 1 |
| | H) 5.00 (s, 2 H) 7.96 (dd, J = 8.5, 1.2 Hz, 1 H) 8.00 (s, 1 H) 8.10 (s, 1 |
| | H) 8.58 (d, J = 8.4 Hz, 1 H) |
| | ¹⁹F NMR (376 MHz, CDCl₃) δ ppm −62.96 (s, 3 F) |
| Final Cpd X69 | ¹H NMR (400 MHz, DMSO-d₆) δ ppm 1.29 (d, J = 6.7 Hz, 6 H), 3.71- |
| | 3.81 (m, 1 H), 3.84 (s, 3 H), 5.28 (s, 2 H), 8.21 (dd, J = 8.5, 1.6 Hz, |
| | 1 H), 8.41 (s, 1 H), 8.46 (s, 1 H), 8.52 (d, J = 8.2 Hz, 1 H), 8.67 (s, 1 |
| | H), 11.14 (s, 1 H) |
| Final Cpd X70 | ¹H NMR (400 MHz, CDCl₃) δ ppm 0.93-1.04 (m, 3 H) 1.21-1.31 |
| | (m, 3 H) 1.39 (dd, J = 6.8, 0.9 Hz, 6 H) 1.43 (br s, 3 H) 1.66-1.87 |
| | (m, 2 H) 2.43 (br d, J = 1.3 Hz, 2 H) 2.48-2.61 (m, 3 H) 2.62-2.73 |
| | (m, 1 H) 3.53 (dquin, J = 13.5, 6.8 Hz, 1 H) 3.77-3.96 (m, 2 H) 4.24 |
| | (br s, 1 H) 4.82-5.03 (m, 2 H) 6.61-6.82 (m, 1 H) 7.97 (d, J = 8.4 |
| | Hz, 1 H) 8.13 (s, 1 H) 8.61 (d, J = 8.1 Hz, 1 H) |
| Final Cpd X71 | ¹H NMR (400 MHz, DMSO-d₆) δ ppm 1.27 (d, J = 6.7 Hz, 6 H) 3.66- |
| | 3.81 (m, 1 H) 4.98 (s, 2 H) 7.31 (dd, J = 9.8, 1.8 Hz, 1 H) 7.80 (d, |
| | J = 9.7 Hz, 1 H) 8.16 (d, J = 11.0 Hz, 1 H) 8.23 (s, 1 H) 9.21 (s, 1 H) |
| | 9.24 (s, 1 H) 10.57 (s, 1 H) |
| Final Cpd X72 | ¹H NMR (400 MHz, DMSO-d₆) δ ppm 1.29 (d, J = 6.5 Hz, 6 H) 3.77 |
| | (spt, J = 6.7 Hz, 1 H) 5.03 (s, 2 H) 8.01 (d, J = 2.0 Hz, 1 H) 8.22 (dd, |
| | J = 8.2, 1.2 Hz, 1 H) 8.43 (s, 1 H) 8.52 (d, J = 8.6 Hz, 1 H) 9.41 (s, 1 |
| | H) 9.44 (d, J = 2.0 Hz, 1 H) 10.76 (br s, 1 H) |
| | ¹⁹F NMR (376 MHz, DMSO-d₆) δ ppm −61.31 (s, 3 F) |
| Final Cpd X73 | ¹H NMR (400 MHz, DMSO-d₆) δ ppm 1.29 (d, J = 6.7 Hz, 6 H) 3.78 |
| | (spt, J = 6.7 Hz, 1 H) 5.05 (s, 2 H) 7.68 (d, J = 1.3 Hz, 1 H) 7.94 (d, |
| | J = 1.3 Hz, 1 H) 8.22 (dd, J = 8.4, 1.3 Hz, 1 H) 8.43 (s, 1 H) 8.49 (d, |
| | J = 2.7 Hz, 1 H) 8.53 (d, J = 8.4 Hz, 1 H) 9.48 (d, J = 2.6 Hz, 1 H) |
| | 10.70 (s, 1 H) |
| | ¹⁹F NMR (376 MHz, DMSO-d₆) δ ppm −61.30 (s, 3 F) |
| Final Cpd X74 | ¹H NMR (400 MHz, DMSO-d₆) δ ppm 1.32 (d, J = 6.8 Hz, 6 H) 2.46- |
| | 2.48 (m, 1 H) 3.16 (s, 3 H) 3.74 (dt, J = 13.4, 6.6 Hz, 1 H) 5.00 (s, 2 |
| | H) 7.14 (d, J = 9.5 Hz, 1 H) 7.44 (d, J = 9.5 Hz, 1 H) 7.59-7.66 (m, 1 |
| | H) 7.82 (s, 1 H) 8.18 (d, J = 8.4 Hz, 1 H) 8.37 (s, 1 H) 8.54 (br d, |
| | J = 8.4 Hz, 1 H) 9.81 (br s, 1 H) |
| Final Cpd X75 | ¹H NMR (400 MHz, CDCl₃) δ ppm 1.36 (s, 3 H) 1.67 (d, J = 6.5 Hz, |
| | 3 H) 1.99 (s, 1 H) 2.04 (dd, J = 8.5, 3.0 Hz, 2 H) 2.49-2.56 (m, 2 H) |
| | 2.90 (d, J = 6.9 Hz, 1 H) 4.01 (h, J = 7.9 Hz, 1 H) 4.85 (s, 2 H) 5.30 (p, |
| | J = 6.6 Hz, 1 H) 6.33 (d, J = 6.6 Hz, 1 H) 8.01 (d, J = 8.4 Hz, 1 H) 8.33 |
| | (s, 1 H) 8.61 (d, J = 8.4 Hz, 1 H) |
| Final Cpd X76 | ¹H NMR (400 MHz, DMSO-d₆) δ ppm 2.70-2.83 (m, 2 H), 3.32- |
| | 3.42 (m, 2 H), 5.04 (s, 2 H), 7.32 (dd, J = 9.8, 1.9 Hz, 1 H), 7.80 (d, |
| | J = 9.7 Hz, 1 H), 8.24 (d, J = 8.5 Hz, 1 H), 8.46 (s, 1 H), 8.51 (d, J = 8.4 |
| | Hz, 1 H), 9.18-9.19 (m, 1 H), 9.23 (s, 1 H), 10.59 (s, 1 H) |
| Final Cpd X77 | ¹H NMR (400 MHz, DMSO-d₆) δ ppm 1.10-1.30 (m, 6 H), 2.25- |
| | 2.46 (m, 4 H), 2.99-3.13 (m, 1 H), 3.74 (spt, J = 6.7 Hz, 1 H), 4.20- |
| | 4.40 (m, 1 H), 4.68-4.76 (m, 2 H), 8.19 (d, J = 8.5 Hz, 1 H), 8.39 (s, |
| | 1 H), 8.44-8.55 (m, 2 H) |
| Final Cpd X78 | ¹H NMR (400 MHz, DMSO-d₆) δ ppm 1.12 (s, 3 H), 1.20-1.30 (m, |
| | 6 H), 1.30-1.42 (m, 4 H), 1.49-1.60 (m, 2 H), 1.67-1.78 (m, 2 H), |
| | 3.64 (dt, J = 7.3, 4.4 Hz, 1 H), 3.68-3.78 (m, 1 H), 4.19 (s, 1 H), |
| | 4.72 (s, 2 H), 7.94 (br d, J = 7.7 Hz, 1 H), 8.15-8.22 (m, 1 H), 8.37 |
| | (s, 1 H), 8.50 (d, J = 8.4 Hz, 1 H) |
| Final Cpd X80 | ¹H NMR (400 MHz, DMSO-d₆) δ ppm 1.29 (d, J = 6.8 Hz, 6 H) 2.37 |
| | (s, 3 H) 3.77 (quin, J = 6.7 Hz, 1 H) 5.02 (s, 2 H) 7.14-7.38 (m, 2 H) |
| | 7.55 (d, J = 9.5 Hz, 1 H) 8.22 (d, J = 8.4 Hz, 1 H) 8.42 (s, 1 H) 8.52 (d, |
| | J = 8.3 Hz, 1 H) 8.81 (s, 1 H) 10.50 (s, 1 H) |
| Final Cpd X81 | ¹H NMR (400 MHz, CDCl₃) δ ppm 1.00 (d, J = 6.2 Hz, 2 H) 1.38 |
| | (dd, J = 6.8, 2.0 Hz, 7 H) 1.43-1.55 (m, 2 H) 1.61-1.72 (m, 1 H) |
| | 1.75-1.89 (m, 1 H) 2.15 (br d, J = 1.8 Hz, 2 H) 2.42-2.62 (m, 4 H) |
| | 2.79 (br d, J = 3.1 Hz, 1 H) 3.45-3.60 (m, 2 H) 3.71 (br s, 1 H) 3.82- |
| | 3.90 (m, 1 H) 4.24 (br s, 1 H) 4.83-5.00 (m, 2 H) 6.62 (br d, J = 6.8 Hz, |
| | 1 H) 7.92-8.01 (m, 1 H) 8.09-8.16 (m, 1 H) 8.61 (d, J = 8.4 Hz, 1 H) |
| Final Cpd X82 | ¹H NMR (400 MHz, DMSO-d₆) δ ppm 1.27 (d, J = 6.5 Hz, 6 H) 3.70 |
| | (spt, J = 6.7 Hz, 1 H) 5.14 (s, 2 H) 7.57 (d, J = 0.8 Hz, 1 H) 7.81 (s, 1 |
| | H) 7.93 (br d, J = 10.2 Hz, 1 H) 8.37 (d, J = 9.8 Hz, 1 H) 9.55 (s, 1 H) |
| | 11.57 (s, 1 H) 12.70 (br s, 1 H) |
| | ¹⁹F NMR (377 MHz, DMSO-d₆) δ ppm −61.79 (s, 3 F) |
| Final Cpd X83 | ¹H NMR (400 MHz, DMSO-d₆, 101° C.) δ ppm 1.31 (d, J = 6.8 Hz, 6 |
| | H) 1.59-1.72 (m, 2 H) 2.21-2.43 (m, 6 H) 2.56-2.77 (m, 3 H) |
| | 3.64-3.72 (m, 1 H) 3.74 (br d, J = 5.9 Hz, 1 H) 3.84-3.93 (m, 1 H) |
| | 4.44 (br s, 1 H) 4.81 (d, J = 1.1 Hz, 2 H) 7.34 (br d, J = 8.4 Hz, 1 H) |
| | 8.12-8.18 (m, 1 H) 8.33 (s, 1 H) 8.52 (d, J = 8.4 Hz, 1 H) |
| | ¹⁹F NMR (377 MHz, DMSO-d₆) δ ppm −[96.05-94.85] (m, 1 F) −79.67 |
| | (br dd, J = 191.7, 5.0 Hz, 1 F) −61.31 (s, 3 F) |
| Final Cpd X84 | ¹H NMR (400 MHz, DMSO-d₆) δ ppm 1.21 (s, 3 H) 1.90-1.99 (m, |
| | 2 H) 2.18-2.27 (m, 2 H) 3.70-3.84 (m, 1 H) 4.69 (d, J = 15.9 Hz, 1 |
| | H) 4.77 (d, J = 15.9 Hz, 1 H) 4.98 (br s, 1 H) 5.61 (q, J = 7.4 Hz, 1 H) |
| | 7.65 (br s, 1 H) 8.23 (dd, J = 8.4, 1.0 Hz, 1 H) 8.35 (br d, J = 7.2 Hz, 1 |
| | H) 8.50 (d, J = 8.4 Hz, 1 H) 8.70 (s, 1 H) |
| Final Cpd X85 | ¹H NMR (400 MHz, DMSO-d₆) δ ppm 1.21 (s, 3 H) 1.49 (d, J = 6.9 |
| | Hz, 3 H) 1.94 (t, J = 9.7 Hz, 2 H) 2.17-2.26 (m, 2 H) 3.69-3.83 (m, |
| | 1 H) 4.73 (q, J = 15.8 Hz, 2 H) 4.92-5.03 (m, 2 H) 8.23 (d, J = 8.4 |
| | Hz, 1 H) 8.31 (d, J = 7.2 Hz, 1 H) 8.50 (d, J = 8.3 Hz, 1 H) 8.63 (s, 1 H) |
| Final Cpd X86 | ¹H NMR (400 MHz, DMSO-d₆) δ ppm 1.27 (d, J = 6.5 Hz, 6 H) 1.31- |
| | 1.50 (m, 2 H) 1.54-1.71 (m, 2 H) 1.74-1.86 (m, 1 H) 1.95 (td, |
| | J = 13.5, 7.8 Hz, 1 H) 3.67-3.90 (m, 3 H) 4.67-4.78 (m, 3 H) 8.01 |
| | (d, J = 7.3 Hz, 1 H) 8.19 (dd, J = 8.6, 1.2 Hz, 1 H) 8.38 (s, 1 H) 8.50 |
| | (d, J = 8.2 Hz, 1 H) |
| | ¹⁹F NMR (377 MHz, DMSO-d₆) δ ppm −61.31 (s, 3 F) |
| Final Cpd X87 | ¹H NMR (400 MHz, DMSO-d₆) δ ppm 1.27-1.32 (m, 6 H) 2.07 (s, |
| | 1 H) 3.77 (quin, J = 6.7 Hz, 1 H) 5.09-5.18 (m, 2 H) 8.13-8.26 (m, |
| | 2 H) 8.41-8.57 (m, 3 H) 11.70-11.78 (m, 1 H) |
| Final Cpd X88 | ¹H NMR (400 MHz, CDCl₃) δ ppm 1.42 (d, J = 6.8 Hz, 6 H) 3.57 |
| | (spt, J = 6.8 Hz, 1 H) 5.11 (s, 2 H) 6.84 (dd, J = 9.6, 1.7 Hz, 1 H) 7.57 |
| | (d, J = 9.6 Hz, 1 H) 8.00 (s, 1 H) 8.05 (dd, J = 8.4, 1.6 Hz, 1 H) 8.19 |
| | (s, 1 H) 8.67 (d, J = 8.4 Hz, 1 H) 9.15 (s, 1 H) 9.20 (s, 1 H) |
| Final Cpd X89 | ¹H NMR (400 MHz, CDCl₃) δ ppm 1.37 (app. dd, J = 6.9, 0.8 Hz, 6 |
| | H) 2.01-2.23 (m, 2 H) 3.06 (t, J = 6.7 Hz, 2 H) 3.51 (spt, J = 6.7 Hz, 1 |
| | H) 3.99 (dd, J = 12.6, 5.7 Hz, 1 H) 4.22 (dd, J = 12.2, 4.5 Hz, 1 H) |
| | 4.50-4.58 (m, 1 H) 4.93 (s, 2 H) 7.21 (br d, J = 7.8 Hz, 1 H) 7.96 (s, |
| | 1 H) 7.97-8.01 (m, 1 H) 8.12 (s, 1 H) 8.58 (d, J = 8.2 Hz, 1 H) |
| | ¹⁹F NMR (377 MHz, CDCl₃) δ ppm −63.00 (s, 3 F) |
| Final Cpd X90 | ¹H NMR (400 MHz, DMSO-d₆) δ ppm 1.29 (d, J = 6.5 Hz, 6 H) 3.78 |
| | (spt, J = 6.7 Hz, 1 H) 5.05 (s, 2 H) 7.24 (dd, J = 7.5, 2.2 Hz, 1 H) 8.13 |
| | (d, J = 1.6 Hz, 1 H) 8.22 (dd, J = 8.4, 1.0 Hz, 1 H) 8.38 (s, 1 H) 8.42 |
| | (s, 1 H) 8.52 (d, J = 8.2 Hz, 1 H) 8.87 (d, J = 7.3 Hz, 1 H) 10.88 (s, 1 H) |
| | ¹⁹F NMR (377 MHz, DMSO-d₆) δ ppm −61.32 (s, 3 F) |
| Final Cpd X91 | ¹H NMR (400 MHz, DMSO-d₆) δ ppm 1.14-1.29 (m, 7 H) 1.31- |
| | 1.44 (m, 1 H) 1.80-1.96 (m, 2 H) 3.73 (spt, J = 6.7 Hz, 1 H) 3.85 |
| | (quin, J = 7.7 Hz, 1 H) 3.96 (quin, J = 8.3 Hz, 1 H) 4.65-4.76 (m, 2 |
| | H) 5.25 (d, J = 7.5 Hz, 1 H) 8.19 (dd, J = 8.4, 1.1 Hz, 1 H) 8.30 (d, |
| | J = 8.3 Hz, 1 H) 8.39 (s, 1 H) 8.50 (d, J = 8.4 Hz, 1 H) |
| | ¹⁹F NMR (376 MHz, DMSO-d₆) δ ppm −61.30 (s, 3 F) |
| Final Cpd X92 | ¹H NMR (400 MHz, DMSO-d₆) δ ppm 1.29 (d, J = 6.7 Hz, 6 H), 2.47- |
| | 2.48 (m, 3 H), 3.77 (quin, J = 6.7 Hz, 1 H), 5.00 (s, 2 H), 7.00 (s, 1 H), |
| | 7.48 (d, J = 1.1 Hz, 1 H), 7.93 (d, J = 1.2 Hz, 1 H), 8.21 (d, J = 8.5 Hz, 1 |
| | H), 8.42 (s, 1 H), 8.52 (d, J = 8.3 Hz, 1 H), 8.98 (s, 1 H), 10.31 (s, 1 H) |
| Final Cpd X93 | ¹H NMR (400 MHz, DMSO-d₆) δ ppm 0.83-0.97 (m, 2 H), 0.97- |
| | 1.06 (m, 2 H), 2.54-2.67 (m, 1 H), 3.59 (s, 3 H), 4.92 (s, 2 H), 6.97 |
| | (d, J = 9.9 Hz, 1 H), 7.91 (br d, J = 9.9 Hz, 1 H), 8.22 (d, J = 8.5 Hz, 1 |
| | H), 8.48 (d, J = 8.3 Hz, 1 H), 8.61 (s, 1 H), 10.83-10.98 (m, 1 H) |
| X-1027 | 1H NMR (400 MHz, CDCl3) d 1.36 (s, 3H), 1.35 (d, J = 6.8 Hz, |
| | 6H), 2.04-1.96 (m, 2H), 2.54-2.46 (m, 2H), 2.10 (s, 1H), 3.42 |
| | (hept, J = 6.7 Hz, 1H), 4.00 (h, J = 7.9 Hz, 1H), 4.83 (s, 2H), 6.46 |
| | (d, J = 7.1 Hz, 1H),. 7.72 (dd, J = 8.6, 1.9 Hz, 1H), 7.84 (d, J = 1.8 |
| | Hz, 1H), 8.41 (d, J = 8.6 Hz, 1H). |
| X-1028 | 1H NMR (400 MHz, DMSO) d 1.25 (d, J = 6.7 Hz, 6H), 3.63 (hept, |
| | J = 6.7 Hz, 1H), 5.03 (s, 2H), 7.93 (dd, J = 8.5, 1.9 Hz, 1H), 7.98 (dd, |
| | J = 5.8, 1.0 Hz, 1H), 8.18 (d, J = 1.9 Hz, 1H), 8.29 (d, J = 8.5 Hz, 1H), |
| | 8.65 (d, J = 5.8 Hz, 1H), 8.92 (d, J = 1.0 Hz, 1H), 11.30 (br s, 1H). |
| X-1029 | 1H NMR (400 MHz, DMSO) d 1.26 (d, J = 6.7 Hz, 6H), 3.64 (hept, |
| | J = 6.7 Hz, 1H), 4.98 (s, 2H), 7.31 (dd, J = 9.8, 1.8 Hz, 1H), 7.79 (d, |
| | J = 9.8 Hz, 1H), 7.93 (dd, J = 8.5, 1.9 Hz, 1H), 8.19 (d, J = 1.9 Hz, |
| | 1H), 8.31 (d, J = 8.5 Hz, 1H), 9.21 (s, 1H), 9.23 (s, 1H), 10.58 (s, 1H). |
| X-1088 | 1H NMR (400 MHz, CHLOROFORM-d) 1.29 (s, 3 H), 1.36 (d, |
| | J = 6.9 Hz, 6 H), 1.84-1.95 (m, 2 H), 2.33-2.43 (m, 2 H), 3.12 (s, 3 |
| | H), 3.43 (spt, J = 6.7 Hz, 1 H), 4.13 (sxt, J = 8.0 Hz, 1 H), 4.83 (s, 2 |
| | H), 6.31 (br d, J = 6.9 Hz, 1 H), 7.88 (dd, J = 8.4, 1.8 Hz, 1 H), 8.02 (d, |
| | J = 2.0 Hz, 1 H), 8.34 (d, J = 8.6 Hz, 1 H). |
| X-1031 | 1H NMR (400 MHz, DMSO-d6) δ ppm 1.23-1.29 (m, 6 H), 3.21- |
| | 3.26 (m, 3 H), 3.58-3.67 (m, 1 H), 4.87-4.92 (m, 2 H), 7.01 (d, |
| | J = 8.4 Hz, 1 H), 7.11 (d, J = 8.1 Hz, 1 H), 7.42-7.44 (m, 1 H), 8.06 |
| | (d, J = 8.6 Hz, 1 H), 8.22 (d, J = 8.2 Hz, 1 H), 8.30 (s, 1 H), 10.11- |
| | 10.14 (m, 1 H), 10.76-10.80 (m, 1 H) |
| X-1080 | 1H NMR (400 MHz, DMSO) d 1.21 (s, 3H), 1.48 (d, J = 6.5 Hz, |
| | 3H), 1.89-1.99 (m, 2H), 2.17-2.28 (m, 2H), 3.68-3.82 (m, 1H), |
| | 4.65 (dd, J = 32.1, 15.8 Hz, 2H), 4.97 (s, 1H), 5.05 (p, J = 6.5 Hz, |
| | 1H), 5.76 (d, J = 5.4 Hz, 1H), 8.03 (dd, J = 8.5, 1.9 Hz, 1H), 8.18 (d, |
| | J = 8.5 Hz, 1H), 8.31 (d, J = 7.2 Hz, 1H), 8.54 (d, J = 1.8 Hz, 1H). |
| X-1081 | 1H NMR (400 MHz, DMSO) d 1.49 (d, J = 6.4 Hz, 3H), 5.14-5.00 |
| | (m, 3H), 5.80 (d, J = 5.4 Hz, 1H), 7.98 (d, J = 5.4 Hz, 1H), 8.05 (d, J = |
| | 8.6 Hz, 1H), 8.26-8.15 (m, 1H), 8.56 (s, 1H), 8.66 (d, J = 5.7 Hz, |
| | 1H), 8.92 (s, 1H), 11.32 (s, 1H), |
| X-1046 | 1H NMR (400 MHz, DMSO) d 1.26 (s, 3H), 1.28 (s, 3H), 3.10 (d, J = |
| | 9.2 Hz, 6H), 3.57 (dt, J = 13.5, 6.7 Hz, 1H), 4.91 (d, J = 9.8 Hz, |
| | 2H), 6.90 (d, J = 2.0 Hz, 1H), 7.24 (dd, J = 9.0, 2.2 Hz, 1H), 7.32 |
| | (dd, J = 9.8, 1.6 Hz, 1H), 7.79 (d, J = 9.8 Hz, 1H), 8.07 (d, J = 9.0 |
| | Hz, 1H), 9.22 (d, J = 9.1 Hz, 2H), 10.52 (s, 1H). |
| X-1048 | 1H NMR (400 MHz, DMSO) d 1.29 (d, J = 6.7 Hz, 6H), 1.41 (s, |
| | 9H), 3.64-3.75 (m, J = 13.6, 6.9 Hz, 1H), 4.97 (s, 2H), 7.32 (d, J = |
| | 9.4 Hz, 1H), 7.79 (d, J = 9.7 Hz, 1H), 7.97 (d, J = 10.3 Hz, 2H), 8.24 |
| | (d, J = 8.2 Hz, 1H), 9.20 (s, 1H), 9.24 (s, 1H), 10.54 (s, 1H). |
| X-1049 | 1H NMR (400 MHz, DMSO) d 1.21 (s, 3H), 1.27 (d, J = 6.7 Hz, |
| | 6H), 1.39 (s, 9H), 1.94 (t, J = 9.9 Hz, 2H), 2.17-2.26 (m, 2H), 3.69- |
| | 3.59 (m, 1H), 3.72-3.83 (m, 1H), 4.64 (s, 2H), 4.95 (s, 1H), 7.90- |
| | 7.96 (m, 2H), 8.22 (d, J = 8.2 Hz, 2H). |
| X-1050 | 1H NMR (400 MHz, DMSO) d 1.28 (d, J = 6.7 Hz, 6H), 1.40 (s, |
| | 9H), 3.68 (dt, J = 13.5, 6.8 Hz, 1H), 5.02 (s, 2H), 7.93-8.01 (m, |
| | 3H), 8.23 (d, J = 8.3 Hz, 1H), 8.65 (d, J = 5.8 Hz, 1H), 8.91 (s, 1H), |
| | 11.28 (s, 1H). |
| X-1082 | 1H NMR (400 MHz, DMSO) d 1.50 (d, J = 6.5 Hz, 3H), 4.98 (q, J = |
| | 16.0 Hz, 2H), 5.10 (p, J = 6.5 Hz, 1H), 5.81 (d, J = 5.5 Hz, 1H), 7.31 |
| | (dd, J = 9.8, 1.8 Hz, 1H), 7.80 (d, J = 9.7 Hz, 1H), 8.06 (dd, J = 8.5, |
| | 1.8 Hz, 1H), 8.21 (d, J = 8.5 Hz, 1H), 8.57 (d, J = 1.8 Hz, 1H), 9.19 |
| | (s, 1H), 9.23 (s, 1H), 10.61 (s, 1H). |
| X-1054 | 1H NMR (400 MHz, CDCl3) d 1.39 (dd, J = 6.8, 1.3 Hz, 6H), 1.49 |
| | (d, J = 6.5 Hz, 3H), 3.30 (s, 3H), 3.51-3.62 (m, 1H), 4.51 (q, J = |
| | 6.5 Hz, 1H), 5.10 (s, 2H), 7.01 (dd, J = 9.7, 1.8 Hz, 1H), 7.67 (d, J = |
| | 9.7 Hz, 1H), 7.76 (dd, J = 8.3, 1.1 Hz, 1H), 7.87 (s, 1H), 8.51 (d, J = |
| | 8.2 Hz, 1H), 8.70 (s, 1H), 9.17 (s, 1H), 9.64 (s, 1H). |
| X-1090 | 1H NMR (400 MHz, CHLOROFORM-d) d 0.92 (t, J = 7.28 Hz, 3H), |
| | 1.36 (d, J = 6.70 Hz, 6H), 1.54-1.64 (m, 4H), 1.86-1.97 (m, 2H), |
| | 1.99-2.17 (m, 1H), 2.47-2.59 (m, 2H), 3.43 (td, J = 6.73, 13.58 Hz, |
| | 1H), 3.98 (sxt, J = 7.81 Hz, 1H), 4.84 (s, 2H), 6.50 (br d, J = 7.40 Hz, |
| | 1H), 7.88 (dd, J = 1.85, 8.55 Hz, 1H), 8.02 (d, J = 1.62 Hz, 1H), 8.00- |
| | 8.04 (m, 1H), 8.28-8.28 (m, 1H), 8.34 (d, J = 8.55 Hz, 1H). |
| X-1018 | 1H NMR (400 MHz, DMSO) d 1.21 (s, 3H), 1.50 (d, J = 6.6 Hz, |
| | 3H), 1.93 (dd, J = 11.1, 9.1 Hz, 2H), 2.17-2.26 (m, 2H), 3.26 (s, |
| | 3H), 3.75 (dt, J = 15.4, 7.8 Hz, 1H), 4.67 (s, 2H), 4.74 (q, J = 6.6 Hz, |
| | 1H), 4.97 (s, 1H), 8.05 (dd, J = 8.6, 1.7 Hz, 1H), 8.21 (d, J = 8.6 Hz, |
| | 1H), 8.29 (d, J = 7.1 Hz, 1H), 8.41 (d, J = 1.6 Hz, 1H). |
| X-1017 | 1H NMR (400 MHz, DMSO) d 1.54 (d, J = 6.6 Hz, 3H), 3.28 (s, |
| | 3H), 4.80 (q, J = 6.6 Hz, 1H), 5.02 (s, 2H), 7.31 (dd, J = 9.8, 1.6 Hz, |
| | 1H), 7.80 (d, J = 9.7 Hz, 1H), 8.09 (dd, J = 8.5, 1.6 Hz, 1H), 8.25 (d, |
| | J = 8.5 Hz, 1H), 8.46 (d, J = 1.6 Hz, 1H), 9.23 (d, J = 8.2 Hz, 2H), |
| | 10.62 (s, 1H). |
| X-1016 | 1H NMR (400 MHz, DMSO) d 1.52 (d, J = 6.6 Hz, 3H), 3.27 (s, 3H), |
| | 4.78 (q, J = 6.6 Hz, 1H), 5.00-5.16 (m, 2H), 7.97 (d, J = 5.7 Hz, 1H), |
| | 8.08 (dd, J = 8.6, 1.8 Hz, 1H), 8.22 (d, J = 8.5 Hz, 1H), 8.44 (d, |
| | J = 1.8 Hz, 1H), 8.66 (d, J = 5.8 Hz, 1H), 8.92 (s, 1H), 11.32 (s, 1H). |
| X-1062 | 1H NMR (400 MHz, CDCl3) d 1.36 (s, 3H), 1.37 (d, J = 6.8 Hz, |
| | 6H), 1.94-2.05 (m, 5H), 2.06 (s, 1H), 2.47-2.54 (m, 2H), 3.53 |
| | (hept, J = 6.8 Hz, 1H), 4.01 (h, J = 7.9 Hz, 1H), 4.86 (s, 2H), 6.45 |
| | (d, J = 7.1 Hz, 1H), 7.87 (dd, J = 8.4, 1.3 Hz, 1H), 8.01 (s, 1H), 8.54 |
| | (d, J = 8.3 Hz, 1H). |
| X-1063 | 1H NMR (400 MHz, DMSO) d 1.29 (d, J = 6.7 Hz, 6H), 2.11 (t, J = |
| | 19.1 Hz, 3H), 3.72 (hept, J = 6.7 Hz, 1H), 5.01 (s, 2H), 7.32 (dd, J = |
| | 9.8, 1.8 Hz, 1H), 7.79 (d, J = 9.7 Hz, 1H), 8.06 (dd, J = 8.4, 1.2 Hz, |
| | 1H), 8.19 (s, 1H), 8.42 (d, J = 8.3 Hz, 1H), 9.20 (s, 1H), 9.23 (s, |
| | 1H), 10.58 (s, 1H). |
| X-1051 | 1H NMR (400 MHz, DMSO) d 1.29 (d, J = 6.7 Hz, 6H), 1.41 (s, |
| | 9H), 3.69 (dt, J = 13.5, 6.8 Hz, 1H), 5.04 (s, 2H), 7.87-8.00 (m, |
| | 3H), 8.24 (d, J = 8.3 Hz, 1H), 8.35 (d, J = 10.0 Hz, 1H), 9.52 (s, |
| | 1H), 11.44 (s, 1H). |
| X-1030 | 1H NMR (400 MHz, DMSO-d6) ppm 1.27 (d, J = 6.8 Hz, 6 H), 3.64 |
| | (spt, J = 6.8 Hz, 1 H), 4.95 (s, 2 H), 6.73 (dd, J = 9.6, 1.7 Hz, 1 H), |
| | 7.31 (s, 1 H), 7.55 (d, J = 9.5 Hz, 1 H), 7.93 (dd, J = 8.6, 2.0 Hz, 1 H), |
| | 8.18 (d, J = 2.0 Hz, 1 H), 8.31 (d, J = 8.6 Hz, 1 H), 8.34 (s, 1 H), 8.98 |
| | (s, 1 H), 10.30 (s, 1 H). |
| X-1032 | 1H NMR (400 MHz, DMSO-d6) δ ppm 0.95 (t, J = 7.1 Hz, 3 H), 1.24 |
| | (d, J = 6.7 Hz, 7 H), 1.37-1.51 (m, 1 H), 1.61-1.73 (m, 2 H), 1.81 |
| | (br t, J = 9.8 Hz, 1 H), 1.93 (br t, J = 10.4 Hz, 1 H), 2.24-2.37 (m, 2 |
| | H), 2.58 (br d, J = 10.8 Hz, 1 H), 2.72 (br d, J = 10.1 Hz, 1 H), 3.60 |
| | (dquin, J = 13.4, 6.7, 6.7, 6.7, 6.7 Hz, 1 H), 3.67-3.78 (m, 1 H), 4.63- |
| | 4.74 (m, 2 H), 7.93 (br d, J = 7.9 Hz, 1 H), 8.03 (dd, J = 8.6, 1.8 Hz, 1 |
| | H), 8.20 (d, J = 8.4 Hz, 1 H), 8.27 (d, J = 1.8 Hz, 1 H) |
| X-1033 | 1H NMR (400 MHz, DMSO-d6) δ ppm 1.24-1.30 (m, 6 H), 3.64 |
| | (quin, J = 6.7 Hz, 1 H), 3.91-3.95 (m, 3 H), 4.95-5.00 (m, 2 H), |
| | 7.11 (dd, J = 8.7, 1.7 Hz, 1 H), 7.69 (d, J = 8.6 Hz, 1 H), 7.94 (d, J = 1.0 |
| | Hz, 1 H), 8.06 (dd, J = 8.4, 1.8 Hz, 1 H), 8.09 (d, J = 1.5 Hz, 1 H), 8.23 |
| | (d, J = 8.5 Hz, 1 H), 8.31 (d, J = 1.8 Hz, 1 H), 10.48 (s, 1 H) |
| X-1022 | 1H NMR (400 MHz, DMSO) d 0.86 (d, J = 2.5 Hz, 2H), 0.93-1.01 |
| | (m, 2H), 1.20 (s, 3H), 1.22 (d, J = 6.9 Hz, 1H), 1.93 (t, J = 9.8 Hz, |
| | 2H), 2.13-2.27 (m, 2H), 3.69-3.81 (m, 1H), 4.59 (s, 2H), 4.97 (s, |
| | 1H), 8.05 (d, J = 8.5 Hz, 1H), 8.17 (d, J = 8.5 Hz, 1H), 8.24 (d, J = |
| | 7.0 Hz, 1H), 8.46 (s, 1H). |
| X-1023 | 1H NMR (400 MHz, DMSO) d 0.86-0.92 (m, 2H), 0.96-1.02 (m, 2H), |
| | 2.51-2.57 (m, 1H), 4.93 (s, 2H), 7.30 (dd, J = 9.8, 1.7 Hz, 1H), 7.79 |
| | (d, J = 9.8 Hz, 1H), 8.08 (dd, J = 8.5, 1.7 Hz, 1H), 8.20 (d, J = 8.5 Hz, |
| | 1H), 8.51 (d, J = 1.5 Hz, 1H), 9.19 (s, 1H), 9.23 (s, 1H), 10.54 (s, 1H). |
| X-1070 | 1H NMR (400 MHz, CDCl3) d 1.07 (d, J = 6.7 Hz, 4H), 1.38 (s, |
| | 3H), 2.02 (dd, J = 13.7, 4.3 Hz, 2H), 2.15-2.26 (m, 1H), 2.46- |
| | 2.56 (m, 2H), 4.01 (h, J = 7.9 Hz, 1H), 4.79 (s, 2H), 6.41 (d, J = 6.9 |
| | Hz, 1H), 7.76 (dd, J = 8.5, 1.9 Hz, 1H), 8.12 (d, J = 1.9 Hz, 1H), |
| | 8.41 (d, J = 8.5 Hz, 1H). |
| X-1002 | 1H NMR (400 MHz, CDCl3) d 1.10 (d, J = 6.6 Hz, 4H), 2.18-2.28 |
| | (m, 1H), 5.04 (s, 2H), 7.79 (dd, J = 8.5, 1.9 Hz, 1H), 8.10 (d, J = |
| | 10.1 Hz, 1H), 8.14 (d, J = 1.9 Hz, 1H), 8.21 (d, J = 10.1 Hz, 1H), |
| | 8.46 (d, J = 8.6 Hz, 1H), 8.91 (s, 1H), 9.36 (s, 1H). |
| X-1003 | 1H NMR (400 MHz, CDCl3) d 1.41 (d, J = 6.8 Hz, 6H), 2.01 (t, J = |
| | 18.2 Hz, 3H), 3.53-3.63 (m, 1H), 5.13 (s, 2H), 7.92 (dd, J = 8.3, |
| | 0.7 Hz, 1H), 8.06 (s, 1H), 8.10 (d, J = 10.1 Hz, 1H), 8.23 (d, J = 10.1 |
| | Hz, 1H), 8.62 (d, J = 8.3 Hz, 1H), 8.90 (s, 1H), 9.50 (s, 1H). |
| X-1064 | 1H NMR (400 MHz, DMSO) d 1.28 (d, J = 6.7 Hz, 6H), 2.10 (t, J = |
| | 19.1 Hz, 3H), 3.66-3.78 (m, 1H), 5.03 (s, 2H), 7.88 (dd, J = 5.9, |
| | 2.7 Hz, 1H), 8.06 (d, J = 8.3 Hz, 1H), 8.19 (s, 1H), 8.41 (d, J = 8.3 |
| | Hz, 1H), 9.04 (d, J = 5.9 Hz, 1H), 9.29 (d, J = 2.0 Hz, 1H), 11.00 (s, 1H). |
| X-1071 | 1H NMR (400 MHz, DMSO) d 0.84-0.94 (m, 2H), 0.95-1.05 (m, 2H), |
| | 2.51-2.58 (m, 1H), 4.93 (s, 2H), 7.30 (dd, J = 9.8, 1.7 Hz, 1H), 7.79 |
| | (d, J = 9.7 Hz, 1H), 7.95 (dd, J = 8.5, 1.9 Hz, 1H), 8.28 (d, J = 8.5 Hz, |
| | 1H), 8.37 (d, J = 1.8 Hz, 1H), 9.19 (s, 1H), 9.23 (s, 1H), 10.55 (s, 1H). |
| X-1004 | 1H NMR (400 MHz, DMSO-d6) ppm 1.26 (d, J = 6.7 Hz, 6 H), 3.64 |
| | (spt, J = 6.7 Hz, 1 H), 5.05 (s, 2 H), 7.91 (br d, J = 10.0 Hz, 1 H), 8.06 |
| | (dd, J = 8.5, 1.8 Hz, 1 H), 8.22 (d, J = 8.5 Hz, 1 H), 8.30-8.37 (m, 2 |
| | H), 9.51 (s, 1 H), 11.47 (br s, 1 H). |
| X-1019 | 1H NMR (400 MHz, DMSO) d 5.07 (s, 2H), 7.09-7.42 (m, 1H), |
| | 7.31 (dd, J = 9.8, 1.5 Hz, 1H), 7.80 (d, J = 9.8 Hz, 1H), 8.18 (d, J = |
| | 8.5 Hz, 1H), 8.25 (s, 1H), 8.28 (d, J = 8.6 Hz, 1H), 9.19 (s, 1H), 9.23 |
| | (s, 1H), 10.69 (s, 1H). |
| X-1001 | 1H NMR (400 MHz, DMSO) d 0.87-0.94 (m, 2H), 0.95-1.03 (m, |
| | 2H), 5.00 (s, 2H), 7.91 (d, J = 10.1 Hz, 1H), 8.08 (dd, J = 8.5, 1.7 |
| | Hz, 1H), 8.19 (d, J = 8.5 Hz, 1H), 8.34 (d, J = 10.1 Hz, 1H), 8.51 (d, |
| | J = 1.6 Hz, 1H), 9.52 (s, 1H), 11.44 (s, 1H), 1H is overlapped with |
| | the solvent. |
| X-1000 | 1H NMR (400 MHz, DMSO) d 5.14 (s, 2H), 7.24 (t, J = 52.5 Hz, |
| | 1H), 7.90 (d, J = 10.0 Hz, 1H), 8.18 (dd, J = 8.6, 1.6 Hz, 1H), 8.24 |
| | (s, 1H), 8.27 (d, J = 8.6 Hz, 1H), 8.35 (d, J = 10.0 Hz, 1H), 9.53 (s, |
| | 1H), 11.54 (s, 1H). |
| X-1035 | ¹H NMR (400 MHz, DMSO-d₆) δ ppm 0.18-0.30 (m, 2 H), 0.34- |
| | 0.41 (m, 2 H), 1.19-1.43 (m, 8 H), 1.55-1.71 (m, 3 H), 2.00-2.22 |
| | (m, 2 H), 2.69 (br d, J = 11.4 Hz, 1 H), 2.84 (br d, J = 9.9 Hz, 1 H), |
| | 3.57-3.69 (m, 2 H), 4.67 (s, 2 H), 7.87 (br d, J = 7.9 Hz, 1 H), 8.04 (dd, |
| | J = 8.5, 1.8 Hz, 1 H), 8.20 (d, J = 8.6 Hz, 1 H), 8.28 (d, J = 1.8 Hz, 1 H) |
| X-1020 | 1H NMR (400 MHz, DMSO) d 1.21 (s, 3H), 1.90-1.98 (m, 2H), |
| | 2.20-2.27 (m, 2H), 3.71-3.81 (m, 1H), 4.74 (s, 2H), 4.99 (s, 1H), |
| | 8.16 (dd, J = 8.6, 1.5 Hz, 1H), 8.22 (br s, 1H), 8.26 (d, J = 8.6 Hz, |
| | 1H), 8.40 (d, J = 7.0 Hz, 1H). |
| X-1024 | 1H NMR (400 MHz, DMSO) d 0.84-0.92 (m, 2H), 0.99 (dd, J = |
| | 10.2, 4.5 Hz, 2H), 2.51-2.55 (m, 1H), 3.59 (s, 3H), 4.88 (s, 2H), |
| | 6.97 (d, J = 9.9 Hz, 1H), 7.91 (d, J = 9.7 Hz, 1H), 8.07 (d, J = 8.5 |
| | Hz, 1H), 8.18 (d, J = 8.5 Hz, 1H), 8.49 (s, 1H), 10.91 (s, 1H). |
| X-1036 | ¹H NMR (400 MHz, DMSO-d₆) δ ppm 1.22-1.28 (m, 6 H), 3.57- |
| | 3.66 (m, 4 H), 4.90-4.96 (m, 2 H), 6.97 (d, J = 9.9 Hz, 1 H), 7.93 |
| | (dt, J = 9.5, 2.0 Hz, 1 H), 8.06 (dd, J = 8.5, 1.8 Hz, 1 H), 8.20 (d, J = 8.5 |
| | Hz, 1 H), 8.29-8.32 (m, 1 H), 10.92-10.98 (m, 1 H) |
| X-1083 | ¹H NMR (400 MHz, DMSO-d₆) δ ppm 0.92-0.99 (m, 3 H), 1.21- |
| | 1.28 (m, 6 H), 1.56-1.69 (m, 2 H), 2.17-2.43 (m, 6 H), 3.61 (dt, |
| | J = 13.5, 6.7 Hz, 1 H), 3.66-3.75 (m, 1 H), 3.81 (br d, J = 6.8 Hz, 1 |
| | H), 4.70-4.80 (m, 3 H), 7.76 (d, J = 8.4 Hz, 1 H), 8.04 (dd, J = 8.5, |
| | 1.8 Hz, 1 H), 8.20 (d, J = 8.5 Hz, 1 H), 8.28 (d, J = 1.8 Hz, 1 H) |
| X-1021 | ¹H NMR (400 MHz, DMSO) d 3.59 (s, 3H), 5.02 (s, 2H), 6.97 (d, J = |
| | 9.9 Hz, 1H), 7.22 (t, J = 52.5 Hz, 1H), 7.91 (d, J = 9.9 Hz, 1H), |
| | 8.17 (dd, J = 8.6, 1.6 Hz, 1H), 8.23 (s, 1H), 8.26 (d, J = 8.6 Hz, 1H), |
| | 11.02 (br s, 1H). |
| X-1084 | 1H NMR (400 MHz, DMSO-d₆) δ ppm 0.18-0.30 (m, 2 H), 0.30- |
| | 0.41 (m, 2 H), 1.25 (dt, J = 6.7, 0.7 Hz, 6 H), 1.51-1.66 (m, 3 H), |
| | 2.40-2.58 (m, 4 H), 3.56-3.66 (m, 1 H), 3.66-3.77 (m, 2 H), 4.69- |
| | 4.79 (m, 3 H), 5.75 (s, 1 H), 7.66 (br d, J = 8.1 Hz, 1 H), 8.04 (dd, |
| | J = 8.5, 1.8 Hz, 1 H), 8.20 (d, J = 8.6 Hz, 1 H), 8.28 (d, J = 1.8 Hz, 1 H) |
| X-1065 | 1H NMR (400 MHz, CDCl3) d 1.40 (d, J = 6.8 Hz, 6H), 2.00 (t, J = |
| | 18.2 Hz, 3H), 3.51-3.61 (m, 1H), 3.65 (s, 3H), 5.03 (s, 2H), 6.94 |
| | (d, J = 9.9 Hz, 1H), 7.90 (d, J = 8.4 Hz, 1H), 8.04 (s, 1H), 8.17 (d, J = |
| | 9.9 Hz, 1H), 8.59 (d, J = 8.3 Hz, 1H), 8.74 (s, 1H). |
| X-1073 | 1H NMR (500 MHz, DMSO-d6) d ppm 1.22-1.34 (m, 10 H) 2.86 |
| | (q, J = 7.6 Hz, 2 H) 3.56-3.72 (m, 1 H) 4.88-5.02 (m, 2 H) 7.32 |
| | (dd, J = 9.8, 1.8 Hz, 1 H) 7.69-7.83 (m, 2 H) 7.88-7.97 (m, 1 H) |
| | 8.18-8.28 (m, 1 H) 9.13-9.27 (m, 2 H) 10.35-10.81 (m, 1 H). |
| X-1006 | 1H NMR (400 MHz, DMSO-d6) ppm 1.29 (d, J = 6.6 Hz, 6 H), 2.11 |
| | (t, J = 19.0 Hz, 3 H), 3.72 (spt, J = 6.8 Hz, 1 H), 5.06 (s, 2 H), 7.73 (d, |
| | J = 1.3 Hz, 1 H), 7.83 (br d, J = 9.7 Hz, 1 H), 8.06 (dd, J = 8.4, 1.3 Hz, 1 |
| | H), 8.11 (d, J = 9.9 Hz, 1 H), 8.16 (s, 1 H), 8.18 (s, 1 H), 8.42 (d, |
| | J = 8.4 Hz, 1 H), 11.23 (br s, 1 H). |
| | 19F NMR (377 MHz, DMSO-d6) ppm −85.51 (q, J = 19.1 Hz, 2 F). |
| X-1007 | 1H NMR (400 MHz, DMSO-d6) d ppm 1.28 (d, J = 6.8 Hz, 6 H) 1.40- |
| | 1.46 (m, 2 H) 1.62-1.71 (m, 2 H) 3.64-3.72 (m, 1 H) 5.06 (s, 2 |
| | H) 7.70 (dd, J = 8.4, 1.5 Hz, 1 H) 7.84 (s, 1 H) 7.92 (br d, J = 10.5 Hz, |
| | 1 H) 8.30 (d, J = 8.4 Hz, 1 H) 8.34 (d, J = 10.8 Hz, 1 H) 9.52 (d, J = 0.7 |
| | Hz, 1 H) 11.34-11.52 (m, 1 H) |
| X-1075 | 1H NMR (400 MHz, CHLOROFORM-d) d ppm 1.06 (br d, J = 4.7 |
| | Hz, 1 H) 1.20-1.30 (m, 1 H) 1.20-1.29 (m, 1 H) 1.22-1.29 (m, 1 |
| | H) 1.35-1.44 (m, 7 H) 1.61-1.76 (m, 4 H) 1.67-1.76 (m, 1 H) |
| | 2.01 (s, 1 H) 2.47 (d, J = 3.2 Hz, 3 H) 3.47-3.54 (m, 1 H) 3.54-3.60 |
| | (m, 1 H) 5.07 (s, 2 H) 7.40 (d, J = 8.6 Hz, 1 H) 7.89 (d, J = 1.5 Hz, 1 |
| | H) 8.15-8.21 (m, 2 H) 8.50 (dd, J = 8.4, 0.8 Hz, 1 H) 9.06 (br s, 1 H) |
| X-1074 | 1H NMR (400 MHz, DMSO-d6) d ppm 1.27 (s, 3 H) 1.25-1.31 (m, |
| | 1 H) 1.29 (s, 3 H) 1.33-1.46 (m, 1 H) 1.62-1.72 (m, 2 H) 3.68 (dt, |
| | J = 13.5, 6.8 Hz, 1 H) 4.99 (s, 2 H) 7.32 (dd, J = 9.8, 1.9 Hz, 1 H) 7.70 |
| | (d, J = 8.0 Hz, 1 H) 7.79 (d, J = 9.8 Hz, 1 H) 7.84 (s, 1 H) 8.31 (d, |
| | J = 8.4 Hz, 1 H) 9.21 (s, 1 H) 9.23 (s, 1 H) 10.56 (s, 1 H) |
| X-1066 | 1H NMR (400 MHz, DMSO-d6) ppm 1.28 (d, J = 6.6 Hz, 6 H), 2.10 |
| | (t, J = 19.1 Hz, 3 H), 3.39 (s, 3 H), 3.71 (spt, J = 6.8 Hz, 1 H), 4.89 (s, |
| | 2 H), 6.40 (d, J = 9.7 Hz, 1 H), 7.40 (dd, J = 9.6, 3.0 Hz, 1 H), 8.05 |
| | (dd, J = 8.4, 1.5 Hz, 1 H), 8.07 (d, J = 2.9 Hz, 1 H), 8.17 (s, 1 H), 8.41 |
| | (d, J = 8.4 Hz, 1 H), 9.97 (s, 1 H). |
| | 19F NMR (377 MHz, CHLOROFORM-d) ppm −85.43 (q, J = 19.1 |
| | Hz, 2 F). |
| X-1008 | 1H NMR (400 MHz, DMSO-d6) ppm 0.88-0.96 (m, 2 H), 1.10- |
| | 1.18 (m, 2 H), 1.27 (d, J = 6.5 Hz, 6 H), 2.17-2.29 (m, 1 H), 3.65 |
| | (spt, J = 6.8 Hz, 1 H), 5.03 (s, 2 H), 7.52 (dd, J = 8.6, 1.6 Hz, 1 H), |
| | 7.77 (d, J = 1.2 Hz, 1 H), 7.92 (d, J = 9.8 Hz, 1 H), 8.16 (d, J = 8.2 Hz, 1 |
| | H), 8.29-8.38 (m, 1 H), 9.51 (d, J = 0.8 Hz, 1 H), 11.43 (br s, 1 H). |
| X-1067 | 1H NMR (400 MHz, DMSO-d6) d ppm 1.30 (d, J = 6.6 Hz, 6 H), |
| | 2.11 (t, J = 19.1 Hz, 3 H), 3.72 (spt, J = 6.7 Hz, 1 H), 4.99 (s, 2 H), |
| | 7.19 (dd, J = 9.5, 2.0 Hz, 1 H), 7.52 (d, J = 1.1 Hz, 1 H), 7.56 (d, J = 9.5 |
| | Hz, 1 H), 7.95 (s, 1 H), 8.06 (dd, J = 8.4, 1.5 Hz, 1 H), 8.19 (s, 1 H), |
| | 8.43 (d, J = 8.1 Hz, 1 H), 9.14 (d, J = 1.1 Hz, 1 H), 10.38 (s, 1 H). |
| | 19F NMR (377 MHz, DMSO-d6) d ppm −85.42 (q, J = 19.1 Hz, 2 F). |
| X-1009 | 1H NMR (400 MHz, DMSO-d6) d ppm 1.29 (d, J = 6.8 Hz, 6 H), |
| | 1.31 (d, J = 7.0 Hz, 6 H), 3.18 (spt, J = 6.9 Hz, 1 H), 3.66 (spt, J = 6.8 |
| | Hz, 1 H), 5.05 (s, 2 H), 7.80 (dd, J = 8.4, 1.3 Hz, 1 H), 7.90 (s, 1 H), |
| | 7.92 (br d, J = 10.1 Hz, 1 H), 8.24 (d, J = 8.1 Hz, 1 H), 8.34 (d, J = 9.9 |
| | Hz, 1 H), 9.52 (s, 1 H), 11.44 (br s, 1 H). |
| X-1085 | 1H NMR (400 MHz, DMSO-d6) d ppm 1.29 (d, J = 6.8 Hz, 6 H), 2.11 (t, |
| | J = 19.1 Hz, 3 H), 3.72 (spt, J = 6.8 Hz, 1 H), 5.07 (s, 2 H), 8.06 (dd, |
| | J = 8.4, 1.5 Hz, 1 H), 8.19 (s, 1 H), 8.42 (d, J = 8.3 Hz, 1 H), 9.18 (d, |
| | J = 1.6 Hz, 1 H), 9.33-9.38 (m, 1 H), 9.42 (s, 1 H), 11.16 (br s, 1 H). |
| | 19F NMR (377 MHz, DMSO-d6) d ppm −85.45 (q, J = 18.1 Hz, 2 F). |
| X-1086 | 1H NMR (400 MHz, DMSO-d6) d ppm 1.28 (d, J = 6.5 Hz, 6 H) 1.31 |
| | (d, J = 6.9 Hz, 6 H) 3.18 (spt, J = 6.9 Hz, 1 H) 3.66 (spt, J = 6.6 Hz, 1 |
| | H) 5.03 (s, 2 H) 7.80 (dd, J = 8.2, 1.2 Hz, 1 H) 7.90 (s, 1 H) 8.24 (d, |
| | J = 8.2 Hz, 1 H) 9.17 (d, J = 1.6 Hz, 1 H) 9.35 (d, J = 1.2 Hz, 1 H) 9.42 |
| | (s, 1 H) 11.12 (s, 1 H) |
| X-1012 | 1H NMR (400 MHz, CDCl3) d 1.40 (d, J = 6.8 Hz, 6H), 2.05 (ddt, J = |
| | 18.7, 9.2, 7.9 Hz, 2H), 2.18-2.27 (m, 2H), 2.44-2.54 (m, 2H), |
| | 3.51-3.62 (m, 1H), 3.71-3.82 (m, 1H), 5.10 (s, 2H), 7.69 (d, J = |
| | 8.1 Hz, 2H), 8.09 (d, J = 10.1 Hz, 1H), 8.24 (d, J = 10.1 Hz, 1H), |
| | 8.48 (d, J = 8.3 Hz, 1H), 8.89 (s, 1H), 9.67 (s, 1H). |
| X-1010 | ¹H NMR (400 MHz, DMSO-d₆) δ ppm 1.27-1.31 (m, 6 H), 1.73- |
| | 1.81 (m, 6 H), 3.68 (quin, J = 6.8 Hz, 1 H), 5.04-5.09 (m, 2 H), 7.89- |
| | 7.96 (m, 2 H), 8.02 (s, 1 H), 8.30-8.36 (m, 2 H), 9.52 (d, J = 0.8 |
| | Hz, 1 H), 11.15-11.58 (m, 1 H) |
| X-1011 | ¹H NMR (400 MHz, DMSO-d₆) δ ppm 1.23-1.35 (m, 6 H), 1.73- |
| | 1.85 (m, 6 H), 3.63-3.73 (m, 1 H), 5.00-5.09 (m, 2 H), 7.73 (d, |
| | J = 1.2 Hz, 1 H), 7.84 (br d, J = 9.7 Hz, 1 H), 7.94 (dd, J = 8.4, 1.6 Hz, |
| | 1 H), 8.02 (d, J = 1.6 Hz, 1 H), 8.11 (d, J = 9.9 Hz, 1 H), 8.16 (s, 1 H), |
| | 8.32 (d, J = 8.3 Hz, 1 H), 11.19-11.27 (m, 1 H) |
| X-1076 | ¹H NMR (400 MHz, DMSO-d₆) δ ppm 1.27-1.31 (m, 6 H), 1.77 |
| | (dt, J = 22.5, 0.7 Hz, 6 H), 3.69 (dt, J = 13.5, 6.8 Hz, 1 H), 4.97-5.00 |
| | (m, 2 H), 7.32 (dd, J = 9.8, 1.9 Hz, 1 H), 7.80 (dt, J = 9.7, 1.0 Hz, 1 H), |
| | 7.94 (dd, J = 8.4, 1.6 Hz, 1 H), 8.01-8.03 (m, 1 H), 8.33 (d, J = 8.3 |
| | Hz, 1 H), 9.20 (s, 1 H), 9.24 (d, J = 0.8 Hz, 1 H), 10.53-10.57 (m, 1 H) |
| X-1077 | ¹H NMR (400 MHz, DMSO-d₆) δ ppm 1.27-1.32 (m, 6 H), 1.72- |
| | 1.83 (m, 6 H), 3.68 (quin, J = 6.7 Hz, 1 H), 4.94-5.00 (m, 2 H), 7.18 |
| | (dd, J = 9.6, 2.0 Hz, 1 H), 7.51 (d, J = 1.1 Hz, 1 H), 7.56 (dt, J = 9.6, 0.9 |
| | Hz, 1 H), 7.92-7.99 (m, 2 H), 8.02 (d, J = 1.7 Hz, 1 H), 8.33 (d, |
| | J = 8.3 Hz, 1 H), 9.13 (dd, J = 2.0, 1.0 Hz, 1 H), 10.33-10.40 (m, 1 H) |
| X-1087 | ¹H NMR (400 MHz, DMSO-d₆) δ ppm 1.25-1.33 (m, 6 H), 1.72- |
| | 1.82 (m, 6 H), 3.68 (quin, J = 6.7 Hz, 1 H), 5.02-5.08 (m, 2 H), 7.94 |
| | (dd, J = 8.4, 1.4 Hz, 1 H), 8.01-8.03 (m, 1 H), 8.32 (d, J = 8.3 Hz, 1 |
| | H), 9.18 (d, J = 1.6 Hz, 1 H), 9.34-9.38 (m, 1 H), 9.43 (s, 1 H), |
| | 11.11-11.17 (m, 1 H) |

### Example B—Pharmaceutical Compositions

A compound of the invention (for instance, a compound of the examples) is brought into association with a pharmaceutically acceptable carrier, thereby providing a pharmaceutical composition comprising such active compound. A therapeutically effective amount of a compound of the invention (e.g. a compound of the examples) is intimately mixed with a pharmaceutically acceptable carrier, in a process for preparing a pharmaceutical composition.

### Example C—Biological Examples

The activity of a compound according to the present invention can be assessed by in vitro methods. A compound the invention exhibits valuable pharmacological properties, e.g. properties susceptible to inhibit NLRP3 activity, for instance as indicated the following test, and are therefore indicated for therapy related to NLRP3 inflammasome activity.

### PBMC Assay

Peripheral venous blood s collected from health individuals and human peripheral blood mononuclear cells (PBMCs) ere isolated from blood by Ficoll Histopaque (Sigma-Aldrich, A0561) density gradient centrifugation. After isolation, PBMCs were stored in liquid nitrogen for later use. Upon thawing, PBMC cell viability was determined in growth medium (RPMI media supplemented with 10% fetal bovine serum, 1% Pen-Strep and 1% L-glutamine). Compounds were spotted in a 1:3 serial dilution in DMSO and diluted to the final concentration in 30 μl medium in 96 well plates (Falcon, 353072). PBMCs were added at a density of 7.5×10⁴ cells per well and incubated for 30 min in a 5% CO₂ incubator at 37° C. LPS stimulation was performed by addition of 100 ng/ml LPS (final concentration, Invivogen, tlrl-smlps) for 6 hrs followed by collection of cellular supernatant and the analysis of IL-1β (μM) and TNF cytokines levels (μM) via MSD technology according to manufacturers' guidelines (MSD, K151A0H).

The IC₅₀ values (for IL-1β) and EC₅₀ values (TNF) were obtained on compounds of the invention/examples, and are depicted in the following table:

| | | IL1β | TNF |
|---|---|---|---|
| | | IC₅₀ | EC₅₀ |
| Number | Compound | (μM) | (μM) |
| Final Cpd 2 | 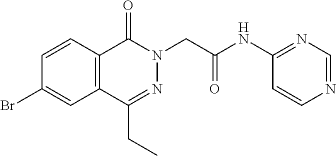 [SMILES: CCc1nn(CC(=O)Nc2ccncn2)c(=O)c2ccc(Br)cc12] | 0.19 | >10 |
| Final Cpd 4 | 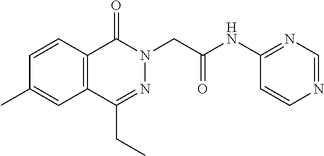 [SMILES: CCc1nn(CC(=O)Nc2ccncn2)c(=O)c2ccc(C)cc12] | 2.91 | >10 |
| Final Cpd 5 | 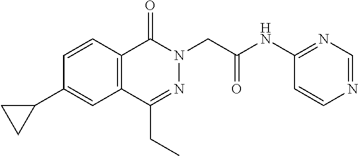 [SMILES: CCc1nn(CC(=O)Nc2ccncn2)c(=O)c2ccc(C3CC3)cc12] | 0.42 | >10 |
| Final Cpd 6 | 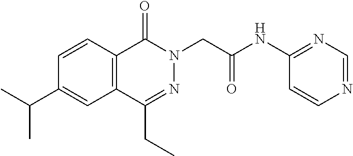 [SMILES: CCc1nn(CC(=O)Nc2ccncn2)c(=O)c2ccc(C(C)C)cc12] | 0.45 | >10 |
| Final Cpd 7 | 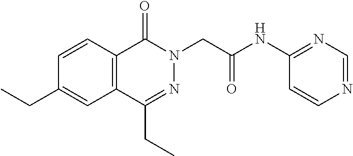 [SMILES: CCc1ccc2c(=O)n(CC(=O)Nc3ccncn3)nc(CC)c2c1] | 0.70 (384 well plate) | >10 |
| Final Cpd 9 | 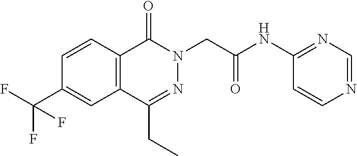 [SMILES: CCc1nn(CC(=O)Nc2ccncn2)c(=O)c2ccc(C(F)(F)F)cc12] | 0.58 | >10 |
| Final Cpd 10 | 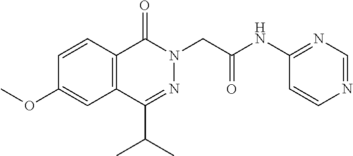 [SMILES: COc1ccc2c(=O)n(CC(=O)Nc3ccncn3)nc(C(C)C)c2c1] | 2.32 | >10 |
| Final Cpd 11 | 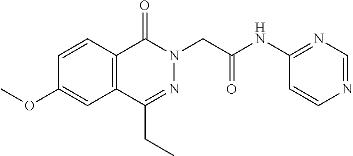 [SMILES: CCc1nn(CC(=O)Nc2ccncn2)c(=O)c2ccc(OC)cc12] | 1.24 | >10 |
| Final Cpd 12 | 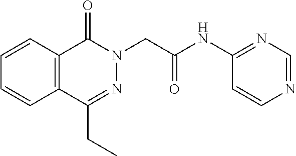 [SMILES: CCc1nn(CC(=O)Nc2ccncn2)c(=O)c2ccccc12] | >20 | >20 |
| Final Cpd 13 | 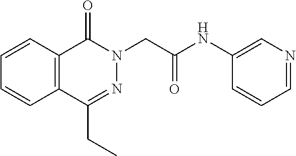 [SMILES: CCc1nn(CC(=O)Nc2cccnc2)c(=O)c2ccccc12] | >20 | >20 |
| Final Cpd 14 | 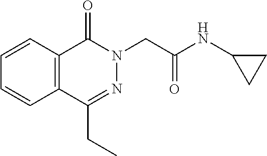 [SMILES: CCc1nn(CC(=O)NC2CC2)c(=O)c2ccccc12] | >20 | >20 |
| Final Cpd 16 | 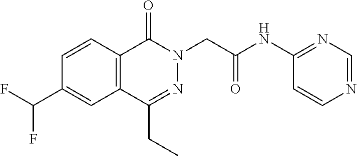 [SMILES: CCc1nn(CC(=O)Nc2ccncn2)c(=O)c2ccc(C(F)F)cc12] | 0.92 (384 well plate) | >10 |
| Final Cpd 17 | 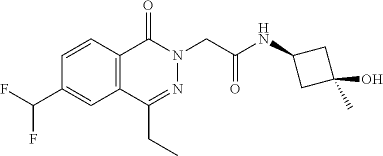 [SMILES: CCc1nn(CC(=O)N[C@H]2C[C@@](C)(O)C2)c(=O)c2ccc(C(F)F)cc12] | 0.54 | >10 |
| Final Cpd 18 | 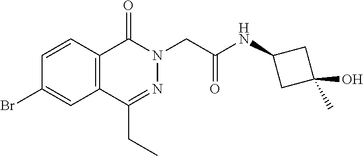 [SMILES: CCc1nn(CC(=O)N[C@H]2C[C@@](C)(O)C2)c(=O)c2ccc(Br)cc12] | 0.03 | >10 |
| Final Cpd 19 | 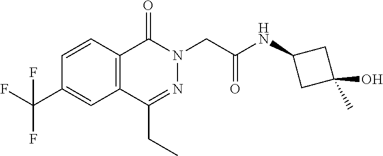 [SMILES: CCc1nn(CC(=O)N[C@H]2C[C@@](C)(O)C2)c(=O)c2ccc(C(F)(F)F)cc12] | 0.14 | >10 |
| Final Cpd 20 | 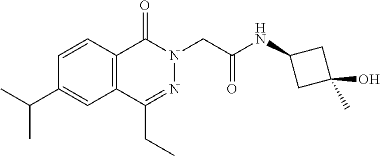 [SMILES: CCc1nn(CC(=O)N[C@H]2C[C@@](C)(O)C2)c(=O)c2ccc(C(C)C)cc12] | 0.24 | >10 |
| Final Cpd 21 | 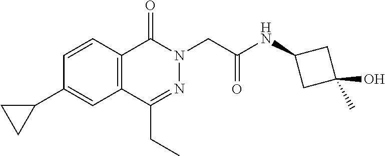 [SMILES: CCc1nn(CC(=O)N[C@H]2C[C@@](C)(O)C2)c(=O)c2ccc(C3CC3)cc12] | 0.29 | >10 |
| Final Cpd 22 | 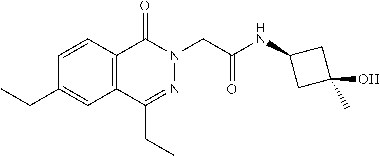 [SMILES: CCc1ccc2c(=O)n(CC(=O)N[C@H]3C[C@@](C)(O)C3)nc(CC)c2c1] | 0.34 | >10 |
| Final Cpd 24 | 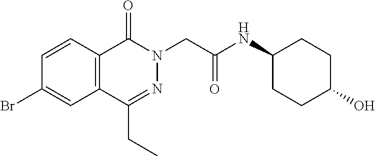 [SMILES: CCc1nn(CC(=O)N[C@H]2CC[C@H](O)CC2)c(=O)c2ccc(Br)cc12] | 0.89 | >10 |
| Final Cpd 26 | 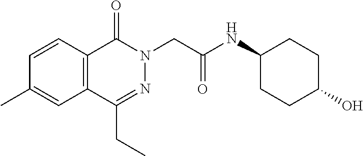 [SMILES: CCc1nn(CC(=O)N[C@H]2CC[C@H](O)CC2)c(=O)c2ccc(C)cc12] | >10 | >10 |
| Final Cpd 27 | 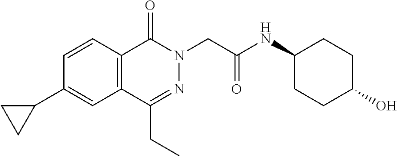 [SMILES: CCc1nn(CC(=O)N[C@H]2CC[C@H](O)CC2)c(=O)c2ccc(C3CC3)cc12] | 1.66 | >10 |
| Final Cpd 28 | 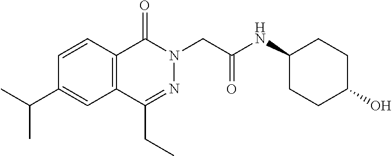 [SMILES: CCc1nn(CC(=O)N[C@H]2CC[C@H](O)CC2)c(=O)c2ccc(C(C)C)cc12] | 0.97 | >10 |
| Final Cpd 29 | 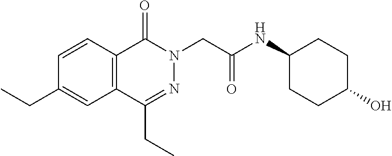 [SMILES: CCc1ccc2c(=O)n(CC(=O)N[C@H]3CC[C@H](O)CC3)nc(CC)c2c1] | 3.59 | >10 |
| Final Cpd 30 | 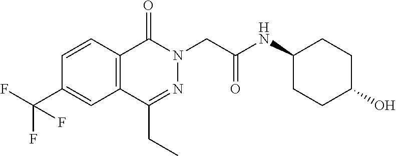 [SMILES: CCc1nn(CC(=O)N[C@H]2CC[C@H](O)CC2)c(=O)c2ccc(C(F)(F)F)cc12] | 1.29 | >10 |
| Final Cpd 1 | 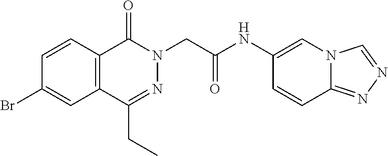 [SMILES: CCc1nn(CC(=O)Nc2ccc3nncn3c2)c(=O)c2ccc(Br)cc12] | 0.115 | >20 |
| Final Cpd 3 | 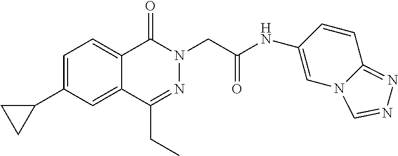 [SMILES: CCc1nn(CC(=O)Nc2ccc3nncn3c2)c(=O)c2ccc(C3CC3)cc12] | 0.124 | 14.86 |
| Final Cpd 8 | 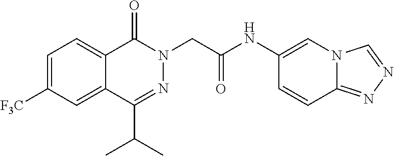 [SMILES: CC(C)c1nn(CC(=O)Nc2ccc3nncn3c2)c(=O)c2ccc(C(F)(F)F)cc12] | 0.076 | 13.65 |
| Final Cpd 15 | 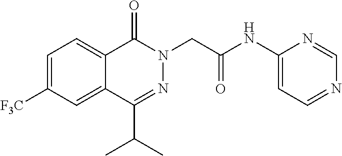 [SMILES: CC(C)c1nn(CC(=O)Nc2ccncn2)c(=O)c2ccc(C(F)(F)F)cc12] | 0.176 | >20 |
| Final Cpd 23 | 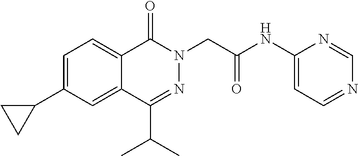 [SMILES: CC(C)c1nn(CC(=O)Nc2ccncn2)c(=O)c2ccc(C3CC3)cc12] | 0.287 | 11.97 |
| Final Cpd 25 | 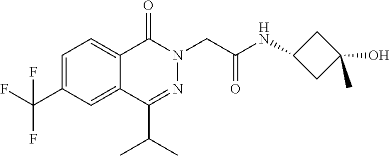 [SMILES: CC(C)c1nn(CC(=O)N[C@H]2C[C@@](C)(O)C2)c(=O)c2ccc(C(F)(F)F)cc12] | 0.058 | 15.37 |
| Final Cpd 31 | 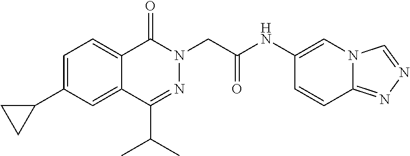 [SMILES: CC(C)c1nn(CC(=O)Nc2ccc3nncn3c2)c(=O)c2ccc(C3CC3)cc12] | 0.103 | 13.61 |
| Final Cpd 32 | 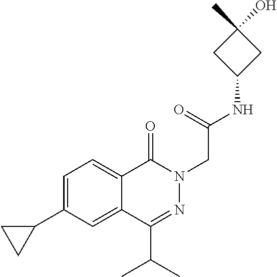 [SMILES: CC(C)c1nn(CC(=O)N[C@H]2C[C@@](C)(O)C2)c(=O)c2ccc(C3CC3)cc12] | 0.213 | >20 |
| Final Cpd 33 | 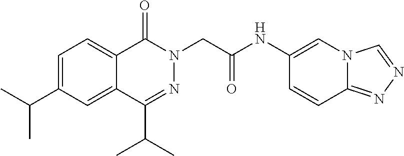 [SMILES: CC(C)c1ccc2c(=O)n(CC(=O)Nc3ccc4nncn4c3)nc(C(C)C)c2c1] | <0.12 | >10 |
| Final Cpd 34 | 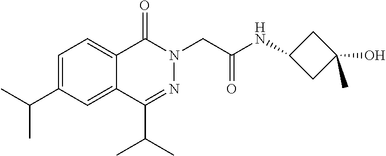 [SMILES: CC(C)c1ccc2c(=O)n(CC(=O)N[C@H]3C[C@@](C)(O)C3)nc(C(C)C)c2c1] | <0.12 | >10 |
| Final Cpd 35 | 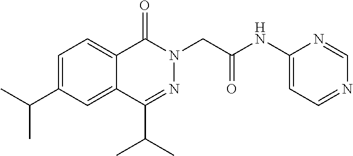 [SMILES: CC(C)c1ccc2c(=O)n(CC(=O)Nc3ccncn3)nc(C(C)C)c2c1] | 0.17 | >10 |
| Final Cpd 36 | 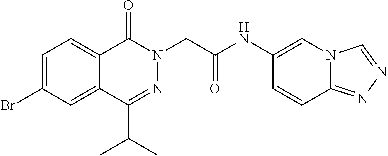 [SMILES: CC(C)c1nn(CC(=O)Nc2ccc3nncn3c2)c(=O)c2ccc(Br)cc12] | <0.025 | >10 |
| Final Cpd 37 | 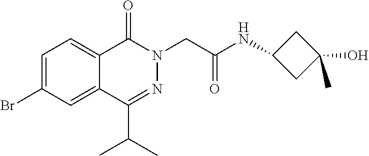 [SMILES: CC(C)c1nn(CC(=O)N[C@H]2C[C@@](C)(O)C2)c(=O)c2ccc(Br)cc12] | <0.025 | >10 |
| Final Cpd 38 | 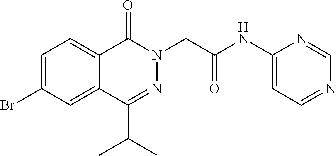 [SMILES: CC(C)c1nn(CC(=O)Nc2ccncn2)c(=O)c2ccc(Br)cc12] | <0.025 | >10 |
| Final Cpd X1 | 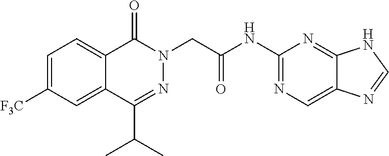 [SMILES: CC(C)c1nn(CC(=O)Nc2ncc3nc[nH]c3n2)c(=O)c2ccc(C(F)(F)F)cc12] | 0.024 | >20 |
| Final Cpd X2 | 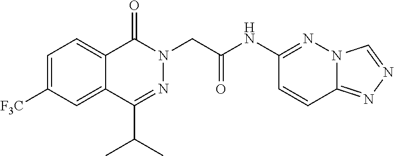 [SMILES: CC(C)c1nn(CC(=O)Nc2ccc3nncn3n2)c(=O)c2ccc(C(F)(F)F)cc12] | 0.03 | 5.58 |
| Final Cpd X3 | 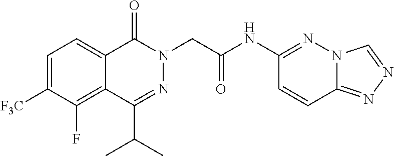 [SMILES: CC(C)c1nn(CC(=O)Nc2ccc3nncn3n2)c(=O)c2ccc(C(F)(F)F)c(F)c12] | 0.051 | 4.01 |
| Final Cpd X4 | 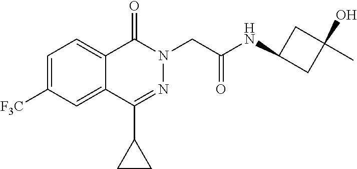 [SMILES: C[C@]1(O)C[C@@H](NC(=O)Cn2nc(C3CC3)c3cc(C(F)(F)F)ccc3c2=O)C1] | 0.06 | >20 |
| Final Cpd X5 | 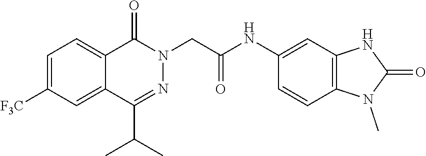 [SMILES: CC(C)c1nn(CC(=O)Nc2ccc3c(c2)[nH]c(=O)n3C)c(=O)c2ccc(C(F)(F)F)cc12] | 0.064 | >20 |
| Final Cpd X6 | 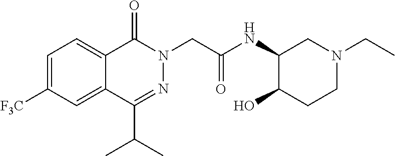 [SMILES: CCN1CC[C@@H](O)[C@@H](NC(=O)Cn2nc(C(C)C)c3cc(C(F)(F)F)ccc3c2=O)C1] | 0.066 | >20 |
| Final Cpd X8 | 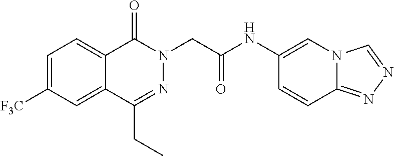 [SMILES: CCc1nn(CC(=O)Nc2ccc3nncn3c2)c(=O)c2ccc(C(F)(F)F)cc12] | 0.075 | 12.17 |
| Final Cpd X9 | 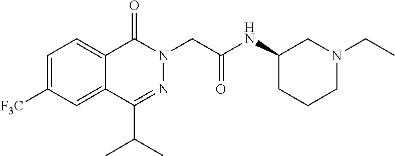 [SMILES: CCN1CCC[C@@H](NC(=O)Cn2nc(C(C)C)c3cc(C(F)(F)F)ccc3c2=O)C1] | 0.09 | >20 |
| Final Cpd X10 | 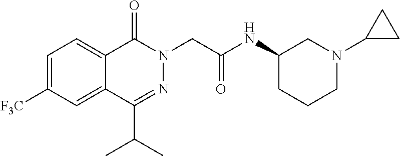 [SMILES: CC(C)c1nn(CC(=O)N[C@@H]2CCCN(C3CC3)C2)c(=O)c2ccc(C(F)(F)F)cc12] | 0.092 | >20 |
| Final Cpd X11 | 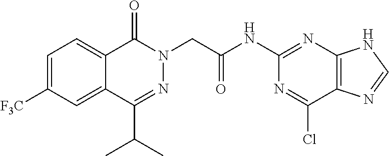 [SMILES: CC(C)c1nn(CC(=O)Nc2nc(Cl)c3nc[nH]c3n2)c(=O)c2ccc(C(F)(F)F)cc12] | 0.105 | >20 |
| Final Cpd X12 | 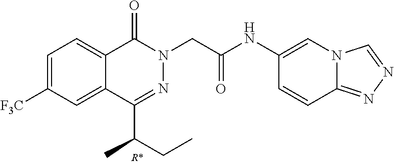 [SMILES: CC[C@@H](C)c1nn(CC(=O)Nc2ccc3nncn3c2)c(=O)c2ccc(C(F)(F)F)cc12] | 0.113 | >20 |
| Final Cpd X13 | 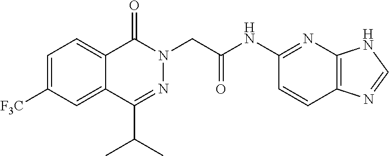 [SMILES: CC(C)c1nn(CC(=O)Nc2ccc3nc[nH]c3n2)c(=O)c2ccc(C(F)(F)F)cc12] | 0.114 | >20 |
| Final Cpd X14 | 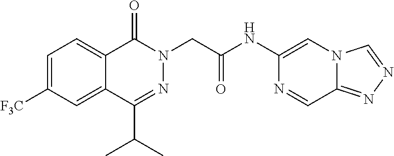 [SMILES: CC(C)c1nn(CC(=O)Nc2cn3cnnc3cn2)c(=O)c2ccc(C(F)(F)F)cc12] | 0.116 | >20 |
| Final Cpd X15 | 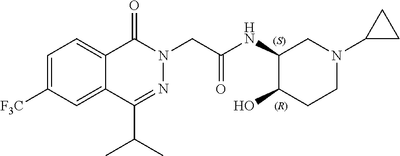 | 0.122 | >20 |
| Final Cpd X16 | 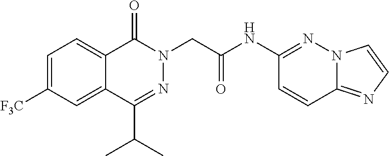 [SMILES: CC(C)c1nn(CC(=O)Nc2ccc3nccn3n2)c(=O)c2ccc(C(F)(F)F)cc12] | 0.137 | >20 |
| Final Cpd X17 | 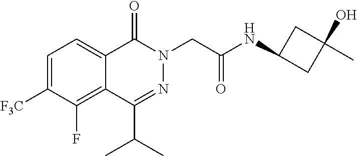 [SMILES: CC(C)c1nn(CC(=O)N[C@H]2C[C@@](C)(O)C2)c(=O)c2ccc(C(F)(F)F)c(F)c12] | 0.147 | >20 |
| Final Cpd X18 | 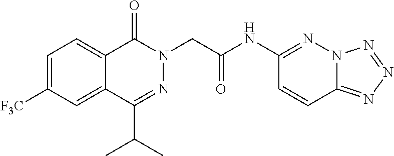 [SMILES: CC(C)c1nn(CC(=O)Nc2ccc3nnnn3n2)c(=O)c2ccc(C(F)(F)F)cc12] | 0.156 | >20 |
| Final Cpd X19 | 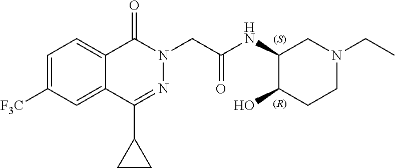 | 0.161 | >20 |
| Final Cpd X20 | 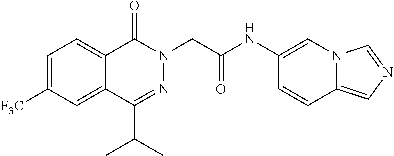 [SMILES: CC(C)c1nn(CC(=O)Nc2ccc3cncn3c2)c(=O)c2ccc(C(F)(F)F)cc12] | 0.164 | 15.53 |
| Final Cpd X21 | 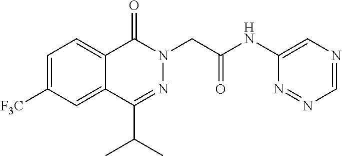 [SMILES: CC(C)c1nn(CC(=O)Nc2cncnn2)c(=O)c2ccc(C(F)(F)F)cc12] | 0.169 | >20 |
| Final Cpd X22 | 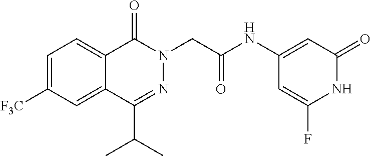 [SMILES: CC(C)c1nn(CC(=O)Nc2cc(F)[nH]c(=O)c2)c(=O)c2ccc(C(F)(F)F)cc12] | 0.17 | >20 |
| Final Cpd X23 | 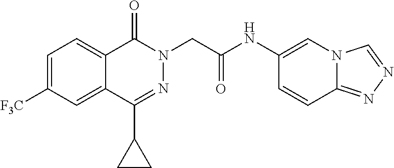 [SMILES: O=C(Cn1nc(C2CC2)c2cc(C(F)(F)F)ccc2c1=O)Nc1ccc2nncn2c1] | 0.172 | >20 |
| Final Cpd X24 | 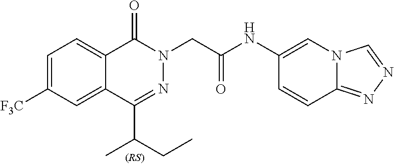 [SMILES: CCC(C)c1nn(CC(=O)Nc2ccc3nncn3c2)c(=O)c2ccc(C(F)(F)F)cc12] | 0.175 | >20 |
| Final Cpd X25 | 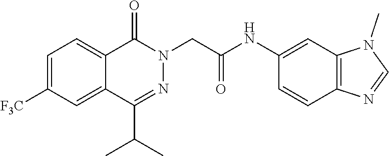 [SMILES: CC(C)c1nn(CC(=O)Nc2ccc3ncn(C)c3c2)c(=O)c2ccc(C(F)(F)F)cc12] | 0.178 | >20 |
| Final Cpd X26 | 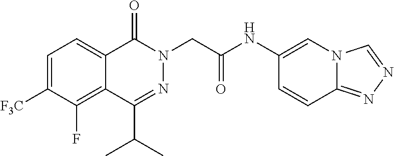 [SMILES: CC(C)c1nn(CC(=O)Nc2ccc3nncn3c2)c(=O)c2ccc(C(F)(F)F)c(F)c12] | 0.185 | >20 |
| Final Cpd X27 | 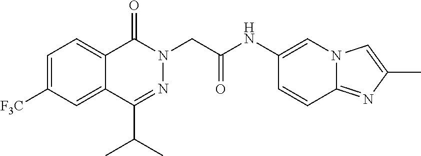 [SMILES: Cc1cn2cc(NC(=O)Cn3nc(C(C)C)c4cc(C(F)(F)F)ccc4c3=O)ccc2n1] | 0.189 | >20 |
| Final Cpd X28 | 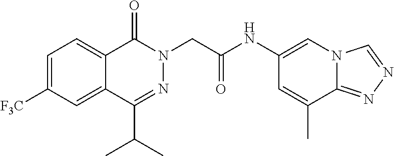 [SMILES: Cc1cc(NC(=O)Cn2nc(C(C)C)c3cc(C(F)(F)F)ccc3c2=O)cn2cnnc12] | 0.193 | >20 |
| Final Cpd X29 | 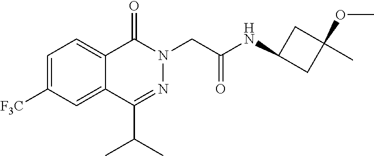 [SMILES: CO[C@]1(C)C[C@H](NC(=O)Cn2nc(C(C)C)c3cc(C(F)(F)F)ccc3c2=O)C1] | 0.216 | >20 |
| Final Cpd X30 | 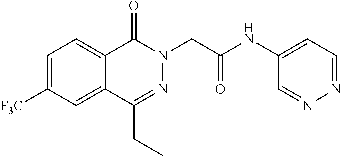 [SMILES: CCc1nn(CC(=O)Nc2ccnnc2)c(=O)c2ccc(C(F)(F)F)cc12] | 0.219 | >20 |
| Final Cpd X31 | 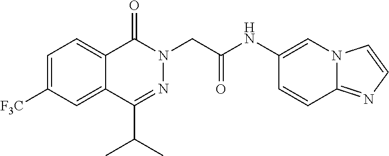 [SMILES: CC(C)c1nn(CC(=O)Nc2ccc3nccn3c2)c(=O)c2ccc(C(F)(F)F)cc12] | 0.233 | >20 |
| Final Cpd X32 | 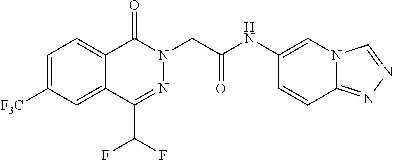 [SMILES: O=C(Cn1nc(C(F)F)c2cc(C(F)(F)F)ccc2c1=O)Nc1ccc2nncn2c1] | 0.235 | >20 |
| Final Cpd X33 | 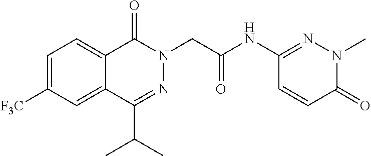 [SMILES: CC(C)c1nn(CC(=O)Nc2ccc(=O)n(C)n2)c(=O)c2ccc(C(F)(F)F)cc12] | 0.252 | >20 |
| Final Cpd X34 | 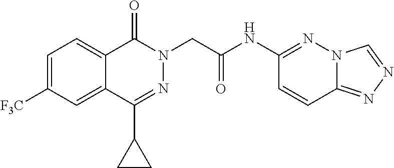 [SMILES: O=C(Cn1nc(C2CC2)c2cc(C(F)(F)F)ccc2c1=O)Nc1ccc2nncn2n1] | 0.255 | >20 |
| Final Cpd X35 | 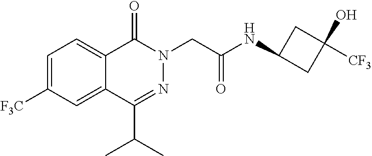 [SMILES: CC(C)c1nn(CC(=O)N[C@H]2C[C@](O)(C(F)(F)F)C2)c(=O)c2ccc(C(F)(F)F)cc12] | 0.303 | >20 |
| Final Cpd X36 | 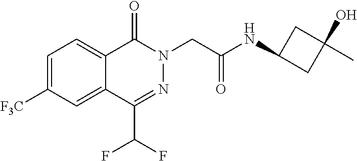 [SMILES: C[C@]1(O)C[C@@H](NC(=O)Cn2nc(C(F)F)c3cc(C(F)(F)F)ccc3c2=O)C1] | 0.304 | >20 |
| Final Cpd X37 | 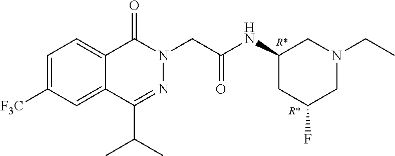 [SMILES: CCN1C[C@H](F)C[C@@H](NC(=O)Cn2nc(C(C)C)c3cc(C(F)(F)F)ccc3c2=O)C1] | 0.316 | >20 |
| Final Cpd X38 | 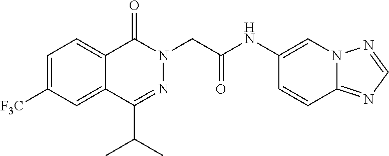 [SMILES: CC(C)c1nn(CC(=O)Nc2ccc3ncnn3c2)c(=O)c2ccc(C(F)(F)F)cc12] | 0.331 | >20 |
| Final Cpd X39 | 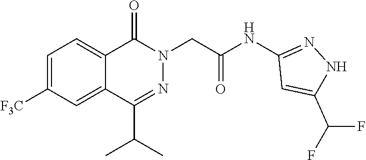 [SMILES: CC(C)c1nn(CC(=O)Nc2cc(C(F)F)[nH]n2)c(=O)c2ccc(C(F)(F)F)cc12] | 0.347 | 12.62 |
| Final Cpd X40 | 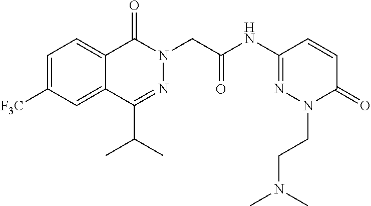 [SMILES: CC(C)c1nn(CC(=O)Nc2ccc(=O)n(CCN(C)C)n2)c(=O)c2ccc(C(F)(F)F)cc12] | 0.349 | >20 |
| Final Cpd X41 | 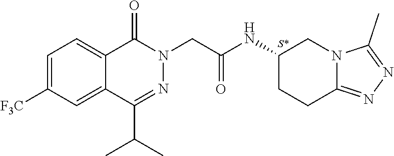 [SMILES: Cc1nnc2n1C[C@@H](NC(=O)Cn1nc(C(C)C)c3cc(C(F)(F)F)ccc3c1=O)CC2] | 0.351 | >20 |
| Final Cpd X42 | 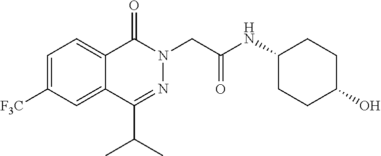 [SMILES: CC(C)c1nn(CC(=O)N[C@H]2CC[C@@H](O)CC2)c(=O)c2ccc(C(F)(F)F)cc12] | 0.353 | 15 |
| Final Cpd X43 | 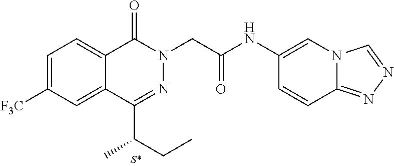 [SMILES: CC[C@H](C)c1nn(CC(=O)Nc2ccc3nncn3c2)c(=O)c2ccc(C(F)(F)F)cc12] | 0.374 | >20 |
| Final Cpd X44 | 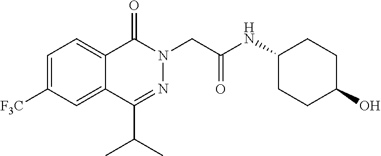 [SMILES: CC(C)c1nn(CC(=O)N[C@H]2CC[C@H](O)CC2)c(=O)c2ccc(C(F)(F)F)cc12] | 0.374 | >20 |
| Final Cpd X45 | 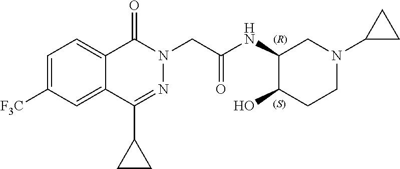 | 0.384 | >20 |
| Final Cpd X46 | 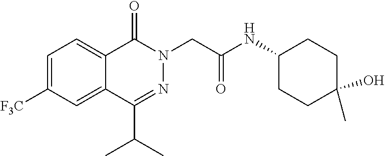 [SMILES: CC(C)c1nn(CC(=O)N[C@H]2CC[C@@](C)(O)CC2)c(=O)c2ccc(C(F)(F)F)cc12] | 0.388 | >20 |
| Final Cpd X47 | 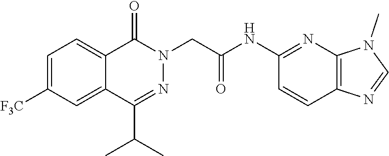 [SMILES: CC(C)c1nn(CC(=O)Nc2ccc3ncn(C)c3n2)c(=O)c2ccc(C(F)(F)F)cc12] | 0.394 | >20 |
| Final Cpd X48 | 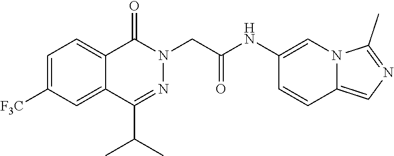 [SMILES: Cc1ncc2ccc(NC(=O)Cn3nc(C(C)C)c4cc(C(F)(F)F)ccc4c3=O)cn12] | 0.398 | >20 |
| Final Cpd X49 | 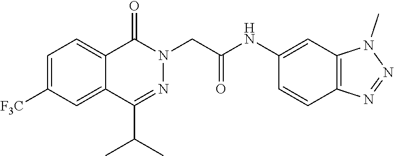 [SMILES: CC(C)c1nn(CC(=O)Nc2ccc3nnn(C)c3c2)c(=O)c2ccc(C(F)(F)F)cc12] | 0.4 | 9.70 |
| Final Cpd X50 | 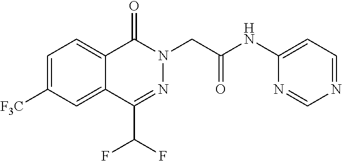 [SMILES: O=C(Cn1nc(C(F)F)c2cc(C(F)(F)F)ccc2c1=O)Nc1ccncn1] | 0.438 | >20 |
| Final Cpd X51 | 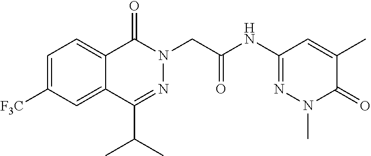 [SMILES: Cc1cc(NC(=O)Cn2nc(C(C)C)c3cc(C(F)(F)F)ccc3c2=O)nn(C)c1=O] | 0.44 | >20 |
| Final Cpd X52 | 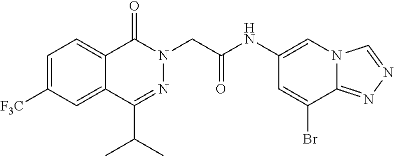 [SMILES: CC(C)c1nn(CC(=O)Nc2cc(Br)c3nncn3c2)c(=O)c2ccc(C(F)(F)F)cc12] | 0.442 | >20 |
| Final Cpd X53 | 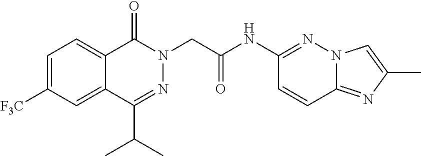 [SMILES: Cc1cn2nc(NC(=O)Cn3nc(C(C)C)c4cc(C(F)(F)F)ccc4c3=O)ccc2n1] | 0.444 | >20 |
| Final Cpd X54 | 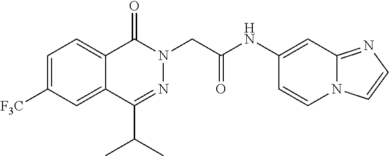 [SMILES: CC(C)c1nn(CC(=O)Nc2ccn3ccnc3c2)c(=O)c2ccc(C(F)(F)F)cc12] | 0.451 | >20 |
| Final Cpd X55 | 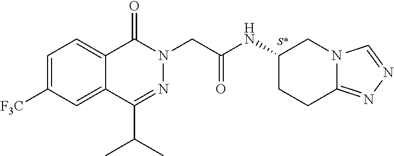 [SMILES: CC(C)c1nn(CC(=O)N[C@H]2CCc3nncn3C2)c(=O)c2ccc(C(F)(F)F)cc12] | 0.46 | >20 |
| Final Cpd X56 | 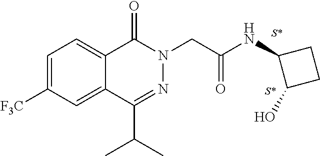 [SMILES: CC(C)c1nn(CC(=O)N[C@H]2CC[C@@H]2O)c(=O)c2ccc(C(F)(F)F)cc12] | 0.493 | >20 |
| Final Cpd X57 | 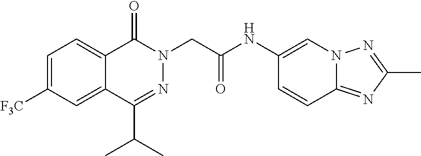 [SMILES: Cc1nc2ccc(NC(=O)Cn3nc(C(C)C)c4cc(C(F)(F)F)ccc4c3=O)cn2n1] | 0.545 | >20 |
| Final Cpd X58 | 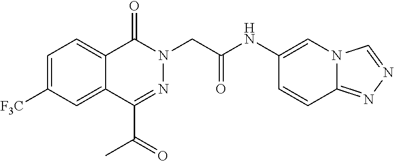 [SMILES: CC(=O)c1nn(CC(=O)Nc2ccc3nncn3c2)c(=O)c2ccc(C(F)(F)F)cc12] | 0.551 | >20 |
| Final Cpd X59 | 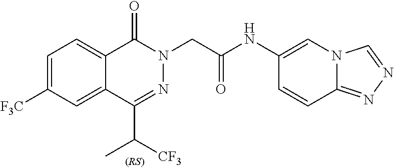 [SMILES: CC(c1nn(CC(=O)Nc2ccc3nncn3c2)c(=O)c2ccc(C(F)(F)F)cc12)C(F)(F)F] | 0.561 | >20 |
| Final Cpd X60 | 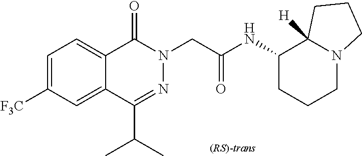 [SMILES: CC(C)c1nn(CC(=O)N[C@H]2CCCN3CCC[C@@H]23)c(=O)c2ccc(C(F)(F)F)cc12] | 0.585 | >20 |
| Final Cpd X61 | 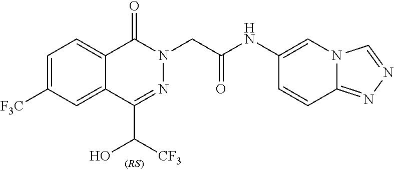 [SMILES: O=C(Cn1nc(C(O)C(F)(F)F)c2cc(C(F)(F)F)ccc2c1=O)Nc1ccc2nncn2c1] | 0.588 | >20 |
| Final Cpd X62 | 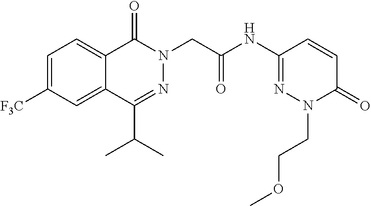 [SMILES: COCCn1nc(NC(=O)Cn2nc(C(C)C)c3cc(C(F)(F)F)ccc3c2=O)ccc1=O] | 0.591 | >20 |
| Final Cpd X63 | 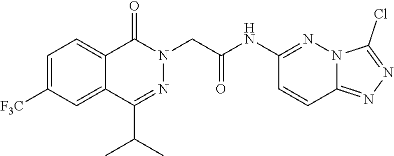 [SMILES: CC(C)c1nn(CC(=O)Nc2ccc3nnc(Cl)n3n2)c(=O)c2ccc(C(F)(F)F)cc12] | 0.6 | >20 |
| Final Cpd X64 | 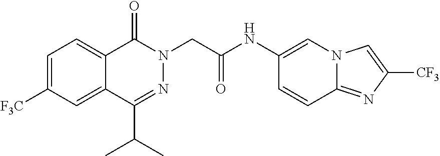 [SMILES: CC(C)c1nn(CC(=O)Nc2ccc3nc(C(F)(F)F)cn3c2)c(=O)c2ccc(C(F)(F)F)cc12] | 0.668 | >20 |
| Final Cpd X65 | 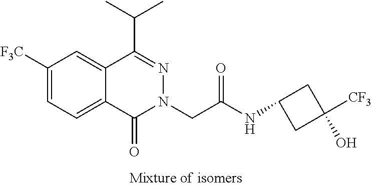 [SMILES: CC(C)c1nn(CC(=O)N[C@H]2C[C@](O)(C(F)(F)F)C2)c(=O)c2ccc(C(F)(F)F)cc12] | 0.668 | >20 |
| Final Cpd X66 | 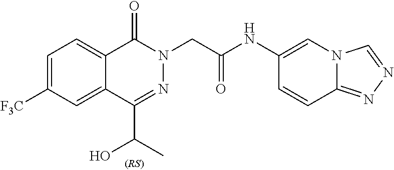 [SMILES: CC(O)c1nn(CC(=O)Nc2ccc3nncn3c2)c(=O)c2ccc(C(F)(F)F)cc12] | 0.67 | >20 |
| Final Cpd X67 | 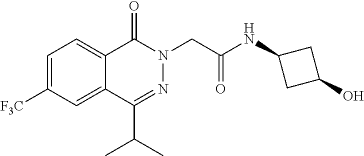 [SMILES: CC(C)c1nn(CC(=O)N[C@H]2C[C@@H](O)C2)c(=O)c2ccc(C(F)(F)F)cc12] | 0.679 | >20 |
| Final Cpd X68 | 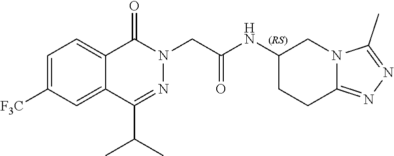 [SMILES: Cc1nnc2n1CC(NC(=O)Cn1nc(C(C)C)c3cc(C(F)(F)F)ccc3c1=O)CC2] | 0.728 | >20 |
| Final Cpd X69 | 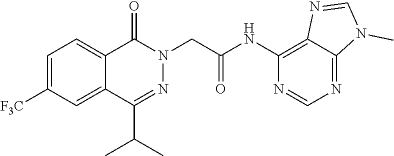 [SMILES: CC(C)c1nn(CC(=O)Nc2ncnc3c2ncn3C)c(=O)c2ccc(C(F)(F)F)cc12] | 0.735 | >20 |
| Final Cpd X70 | 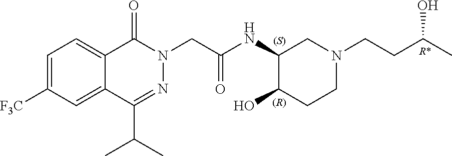 | 0.74 | >20 |
| Final Cpd X71 | 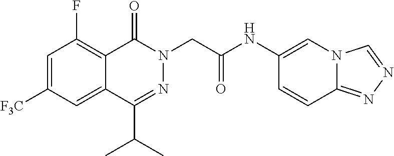 [SMILES: CC(C)c1nn(CC(=O)Nc2ccc3nncn3c2)c(=O)c2c(F)cc(C(F)(F)F)cc12] | 0.751 | >20 |
| Final Cpd X72 | 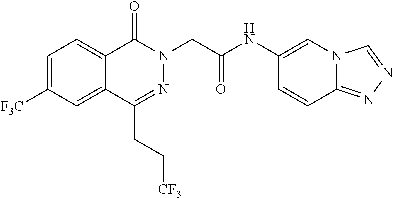 [SMILES: O=C(Cn1nc(CCC(F)(F)F)c2cc(C(F)(F)F)ccc2c1=O)Nc1ccc2nncn2c1] | 0.776 | >20 |
| Final Cpd X73 | 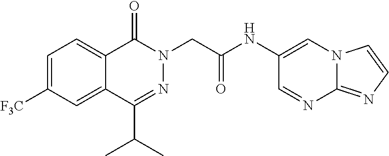 [SMILES: CC(C)c1nn(CC(=O)Nc2cnc3nccn3c2)c(=O)c2ccc(C(F)(F)F)cc12] | 0.792 | >20 |
| Final Cpd X74 | 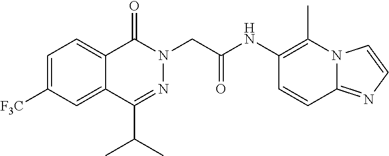 [SMILES: Cc1c(NC(=O)Cn2nc(C(C)C)c3cc(C(F)(F)F)ccc3c2=O)ccc2nccn12] | 0.804 | >20 |
| Final Cpd X75 | 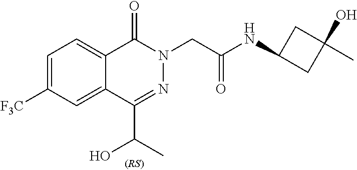 [SMILES: CC(O)c1nn(CC(=O)N[C@H]2C[C@@](C)(O)C2)c(=O)c2ccc(C(F)(F)F)cc12] | 0.818 | >20 |
| Final Cpd X76 | 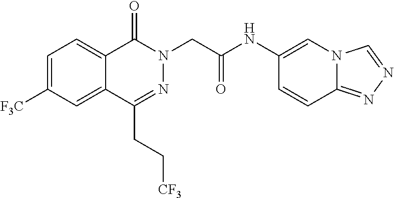 [SMILES: O=C(Cn1nc(CCC(F)(F)F)c2cc(C(F)(F)F)ccc2c1=O)Nc1ccc2nncn2c1] | 0.829 | >20 |
| Final Cpd X77 | 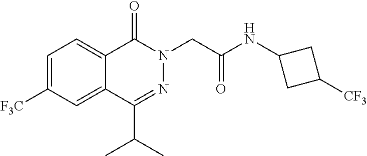 [SMILES: CC(C)c1nn(CC(=O)NC2CC(C(F)(F)F)C2)c(=O)c2ccc(C(F)(F)F)cc12] | 0.83 | >20 |
| Final Cpd X78 | 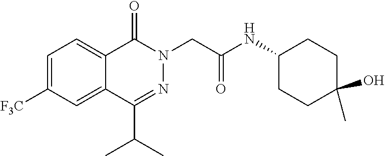 [SMILES: CC(C)c1nn(CC(=O)N[C@H]2CC[C@](C)(O)CC2)c(=O)c2ccc(C(F)(F)F)cc12] | 0.861 | >20 |
| Final Cpd X79 | 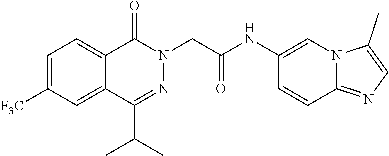 [SMILES: Cc1cnc2ccc(NC(=O)Cn3nc(C(C)C)c4cc(C(F)(F)F)ccc4c3=O)cn12] | 0.865 | >20 |
| Final Cpd X80 | 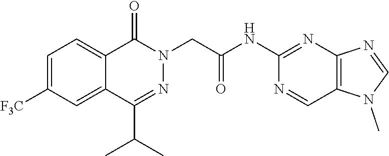 [SMILES: CC(C)c1nn(CC(=O)Nc2ncc3c(ncn3C)n2)c(=O)c2ccc(C(F)(F)F)cc12] | 0.866 | >20 |
| Final Cpd X81 | 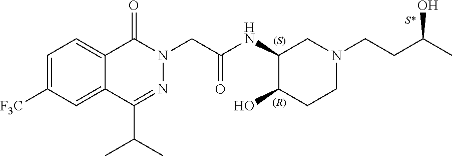 | 0.879 | >20 |
| Final Cpd X82 | 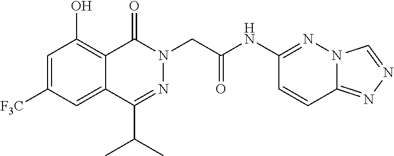 [SMILES: CC(C)c1nn(CC(=O)Nc2ccc3nncn3n2)c(=O)c2c(O)cc(C(F)(F)F)cc12] | 0.888 | 12.86 |
| Final Cpd X83 | 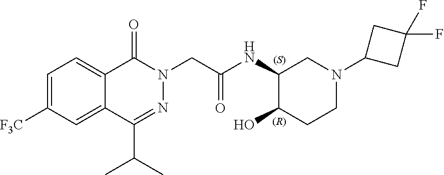 | 0.91 | 12.86 |
| Final Cpd X84 | 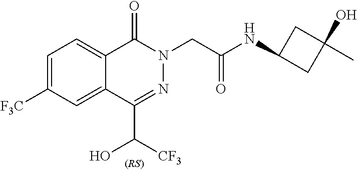 [SMILES: C[C@]1(O)C[C@@H](NC(=O)Cn2nc(C(O)C(F)(F)F)c3cc(C(F)(F)F)ccc3c2=O)C1] | 0.911 | >20 |
| Final Cpd X85 | 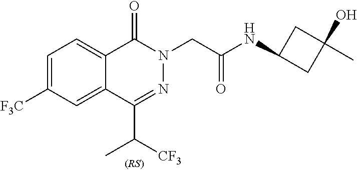 [SMILES: CC(c1nn(CC(=O)N[C@H]2C[C@@](C)(O)C2)c(=O)c2ccc(C(F)(F)F)cc12)C(F)(F)F] | 0.915 | >20 |
| Final Cpd X86 | 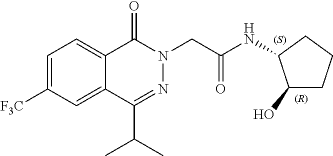 | 0.915 | >20 |
| Final Cpd X87 | 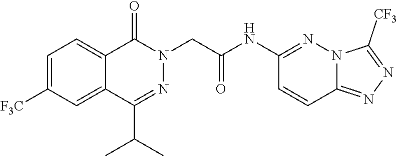 [SMILES: CC(C)c1nn(CC(=O)Nc2ccc3nnc(C(F)(F)F)n3n2)c(=O)c2ccc(C(F)(F)F)cc12] | 0.916 | >20 |
| Final Cpd X88 | 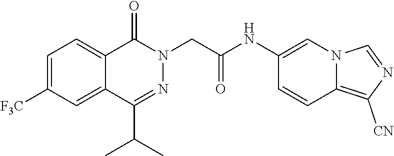 [SMILES: CC(C)c1nn(CC(=O)Nc2ccc3c(C#N)ncn3c2)c(=O)c2ccc(C(F)(F)F)cc12] | 0.924 | >20 |
| Final Cpd X89 | 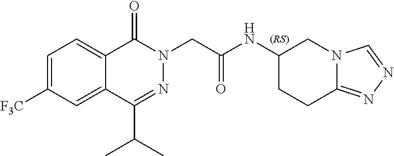 [SMILES: CC(C)c1nn(CC(=O)NC2CCc3nncn3C2)c(=O)c2ccc(C(F)(F)F)cc12] | 0.924 | >20 |
| Final Cpd X90 | 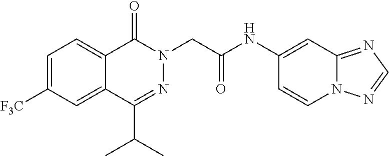 [SMILES: CC(C)c1nn(CC(=O)Nc2ccn3ncnc3c2)c(=O)c2ccc(C(F)(F)F)cc12] | 0.936 | >20 |
| Final Cpd X91 | 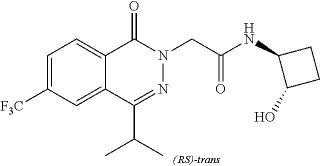 [SMILES: CC(C)c1nn(CC(=O)N[C@H]2CC[C@@H]2O)c(=O)c2ccc(C(F)(F)F)cc12] | 0.954 | >20 |
| Final Cpd X92 | 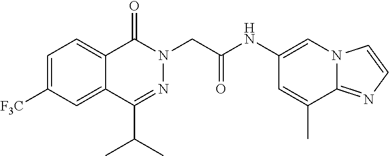 [SMILES: Cc1cc(NC(=O)Cn2nc(C(C)C)c3cc(C(F)(F)F)ccc3c2=O)cn2ccnc12] | 0.97 | >20 |
| Final Cpd X93 | 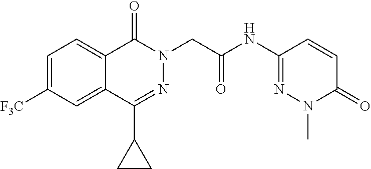 [SMILES: Cn1nc(NC(=O)Cn2nc(C3CC3)c3cc(C(F)(F)F)ccc3c2=O)ccc1=O] | 1.007 | >20 |
| Final Cpd X94 | 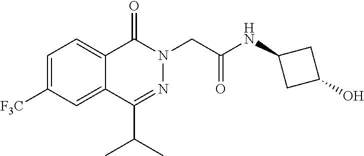 [SMILES: CC(C)c1nn(CC(=O)N[C@H]2C[C@H](O)C2)c(=O)c2ccc(C(F)(F)F)cc12] | 1.072 | >20 |
| Final Cpd X95 | 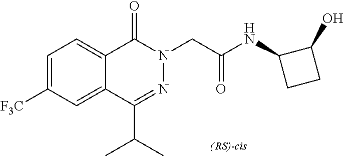 [SMILES: CC(C)c1nn(CC(=O)N[C@@H]2CC[C@@H]2O)c(=O)c2ccc(C(F)(F)F)cc12] | 1.103 | >20 |
| Final Cpd X96 | 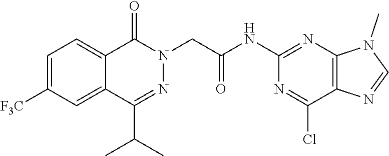 [SMILES: CC(C)c1nn(CC(=O)Nc2nc(Cl)c3ncn(C)c3n2)c(=O)c2ccc(C(F)(F)F)cc12] | 1.111 | >20 |
| Final Cpd X97 | 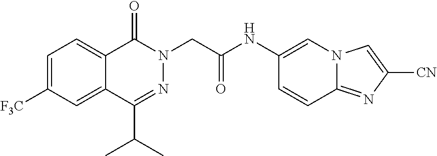 [SMILES: CC(C)c1nn(CC(=O)Nc2ccc3nc(C#N)cn3c2)c(=O)c2ccc(C(F)(F)F)cc12] | 1.123 | >20 |
| Final Cpd X98 | 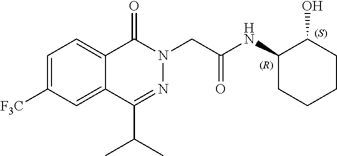 | 1.15 | >20 |
| Final Cpd X99 | 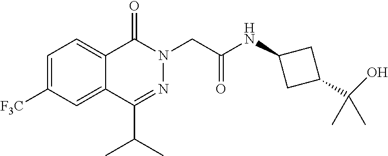 [SMILES: CC(C)c1nn(CC(=O)N[C@H]2C[C@H](C(C)(C)O)C2)c(=O)c2ccc(C(F)(F)F)cc12] | 1.162 | >20 |
| Final Cpd X100 | 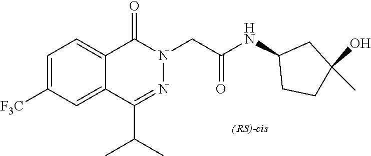 [SMILES: CC(C)c1nn(CC(=O)N[C@@H]2CC[C@](C)(O)C2)c(=O)c2ccc(C(F)(F)F)cc12] | 1.257 | >20 |
| Final Cpd X101 | 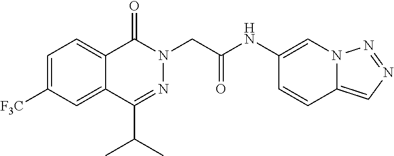 [SMILES: CC(C)c1nn(CC(=O)Nc2ccc3cnnn3c2)c(=O)c2ccc(C(F)(F)F)cc12] | 1.278 | >20 |
| Final Cpd X102 | 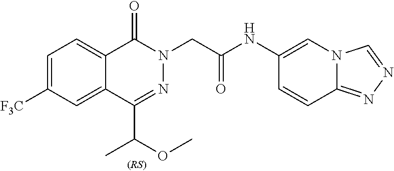 [SMILES: COC(C)c1nn(CC(=O)Nc2ccc3nncn3c2)c(=O)c2ccc(C(F)(F)F)cc12] | 1.297 | >20 |
| Final Cpd X103 | 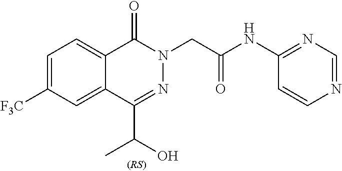 [SMILES: CC(O)c1nn(CC(=O)Nc2ccncn2)c(=O)c2ccc(C(F)(F)F)cc12] | 1.317 | >20 |
| Final Cpd X104 | 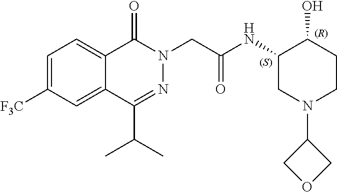 | 1.406 | >20 |
| Final Cpd X105 | 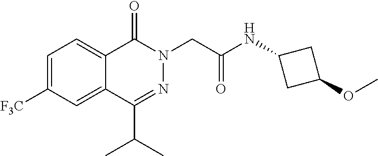 [SMILES: CO[C@H]1C[C@H](NC(=O)Cn2nc(C(C)C)c3cc(C(F)(F)F)ccc3c2=O)C1] | 1.518 | >20 |
| Final Cpd X106 | 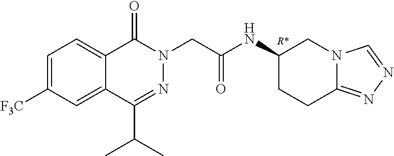 [SMILES: CC(C)c1nn(CC(=O)N[C@@H]2CCc3nncn3C2)c(=O)c2ccc(C(F)(F)F)cc12] | 1.61 | >20 |
| Final Cpd X107 | 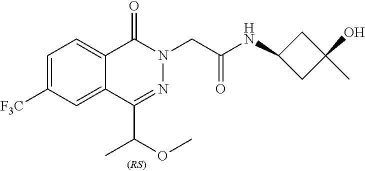 [SMILES: COC(C)c1nn(CC(=O)N[C@H]2C[C@@](C)(O)C2)c(=O)c2ccc(C(F)(F)F)cc12] | 1.611 | >20 |
| Final Cpd X108 | 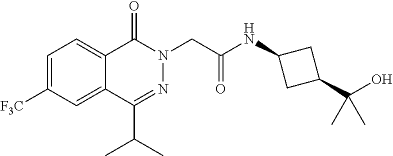 [SMILES: CC(C)c1nn(CC(=O)N[C@H]2C[C@@H](C(C)(C)O)C2)c(=O)c2ccc(C(F)(F)F)cc12] | 1.627 | >20 |
| Final Cpd X109 | 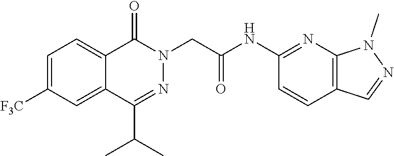 [SMILES: CC(C)c1nn(CC(=O)Nc2ccc3cnn(C)c3n2)c(=O)c2ccc(C(F)(F)F)cc12] | 1.632 | 19.2 |
| Final Cpd X110 | 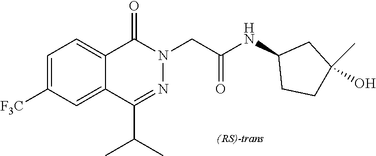 [SMILES: CC(C)c1nn(CC(=O)N[C@@H]2CC[C@@](C)(O)C2)c(=O)c2ccc(C(F)(F)F)cc12] | 1.635 | >20 |
| Final Cpd X111 | 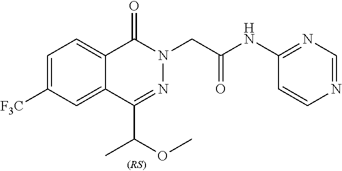 [SMILES: COC(C)c1nn(CC(=O)Nc2ccncn2)c(=O)c2ccc(C(F)(F)F)cc12] | 1.697 | 7.93 |
| Final Cpd X112 | 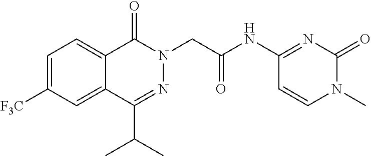 [SMILES: CC(C)c1nn(CC(=O)Nc2ccn(C)c(=O)n2)c(=O)c2ccc(C(F)(F)F)cc12] | 1.728 | >20 |
| Final Cpd X113 | 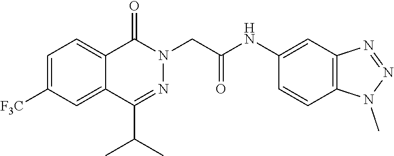 [SMILES: CC(C)c1nn(CC(=O)Nc2ccc3c(c2)nnn3C)c(=O)c2ccc(C(F)(F)F)cc12] | 1.865 | >20 |
| Final Cpd X114 | 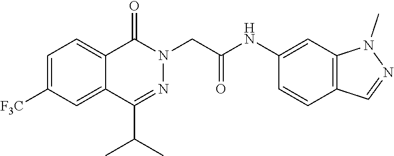 [SMILES: CC(C)c1nn(CC(=O)Nc2ccc3cnn(C)c3c2)c(=O)c2ccc(C(F)(F)F)cc12] | 1.936 | >20 |
| Final Cpd X115 | 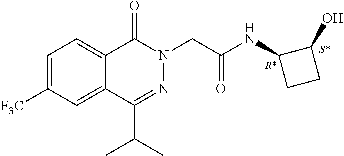 [SMILES: CC(C)c1nn(CC(=O)N[C@@H]2CC[C@@H]2O)c(=O)c2ccc(C(F)(F)F)cc12] | 2.021 | >20 |
| Final Cpd X116 | 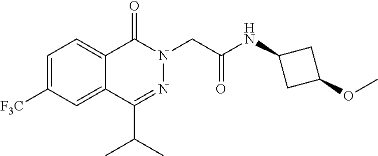 [SMILES: CO[C@H]1C[C@@H](NC(=O)Cn2nc(C(C)C)c3cc(C(F)(F)F)ccc3c2=O)C1] | 2.089 | >20 |
| Final Cpd X117 | 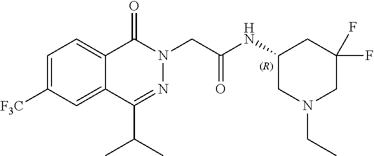 | 2.114 | >20 |
| Final Cpd X118 | 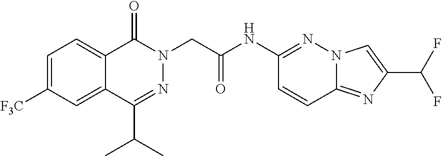 [SMILES: CC(C)c1nn(CC(=O)Nc2ccc3nc(C(F)F)cn3n2)c(=O)c2ccc(C(F)(F)F)cc12] | 2.126 | >20 |
| Final Cpd X119 | 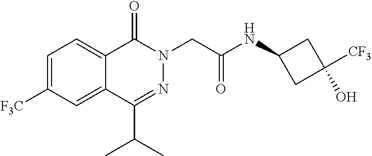 [SMILES: CC(C)c1nn(CC(=O)N[C@H]2C[C@@](O)(C(F)(F)F)C2)c(=O)c2ccc(C(F)(F)F)cc12] | 2.128 | >20 |
| Final Cpd X120 | 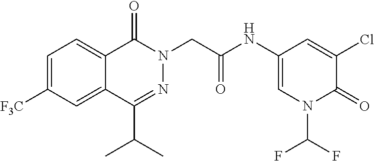 [SMILES: CC(C)c1nn(CC(=O)Nc2cc(Cl)c(=O)n(C(F)F)c2)c(=O)c2ccc(C(F)(F)F)cc12] | 2.57 | >20 |
| Final Cpd X121 | 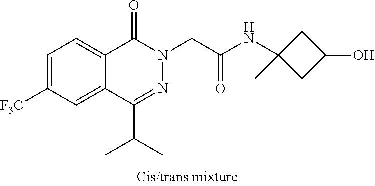 [SMILES: CC(C)c1nn(CC(=O)NC2(C)CC(O)C2)c(=O)c2ccc(C(F)(F)F)cc12] | 2.65 | >20 |
| Final Cpd X122 | 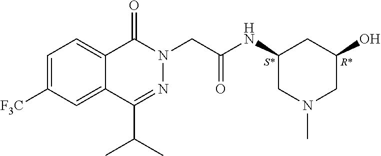 [SMILES: CC(C)c1nn(CC(=O)N[C@H]2C[C@@H](O)CN(C)C2)c(=O)c2ccc(C(F)(F)F)cc12] | 2.675 | >20 |
| Final Cpd X123 | 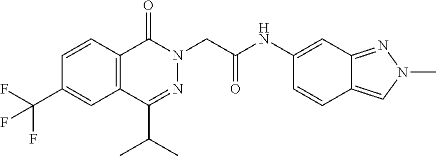 [SMILES: CC(C)c1nn(CC(=O)Nc2ccc3cn(C)nc3c2)c(=O)c2ccc(C(F)(F)F)cc12] | 2.697 | >20 |
| Final Cpd X124 | 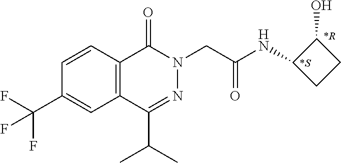 [SMILES: CC(C)c1nn(CC(=O)N[C@H]2CC[C@H]2O)c(=O)c2ccc(C(F)(F)F)cc12] | 2.756 | >20 |
| Final Cpd X125 | 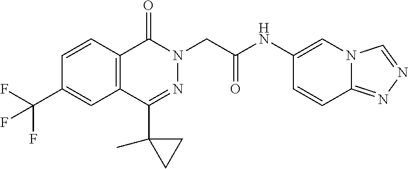 [SMILES: CC1(c2nn(CC(=O)Nc3ccc4nncn4c3)c(=O)c3ccc(C(F)(F)F)cc23)CC1] | 2.883 | >20 |
| Final Cpd X126 | 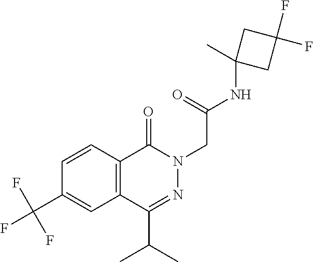 [SMILES: CC(C)c1nn(CC(=O)NC2(C)CC(F)(F)C2)c(=O)c2ccc(C(F)(F)F)cc12] | 2.938 | >20 |
| Final Cpd X127 | 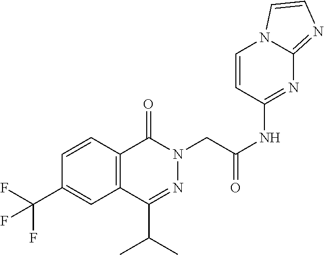 [SMILES: CC(C)c1nn(CC(=O)Nc2ccn3ccnc3n2)c(=O)c2ccc(C(F)(F)F)cc12] | 2.941 | >20 |
| Final Cpd X128 | 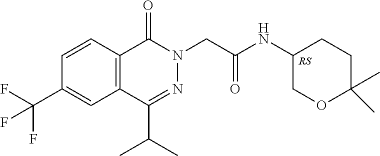 [SMILES: CC(C)c1nn(CC(=O)NC2CCC(C)(C)OC2)c(=O)c2ccc(C(F)(F)F)cc12] | 2.954 | >20 |
| Final Cpd X129 | 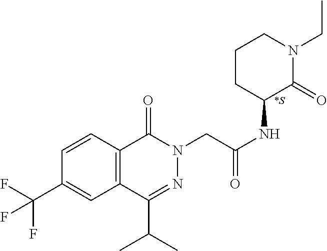 [SMILES: CCN1CCC[C@H](NC(=O)Cn2nc(C(C)C)c3cc(C(F)(F)F)ccc3c2=O)C1=O] | 3.068 | >20 |
| Final Cpd X130 | 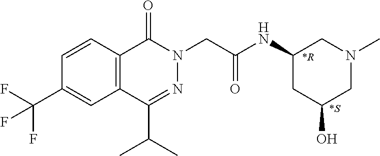 [SMILES: CC(C)c1nn(CC(=O)N[C@@H]2C[C@H](O)CN(C)C2)c(=O)c2ccc(C(F)(F)F)cc12] | 3.079 | >20 |
| Final Cpd X131 | 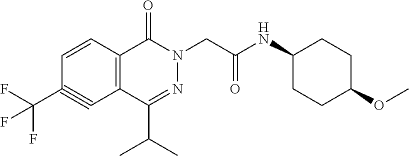 | 3.087 | >20 |
| Final Cpd X132 | 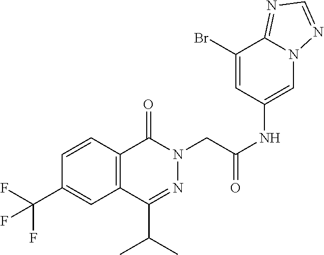 [SMILES: CC(C)c1nn(CC(=O)Nc2cc(Br)c3ncnn3c2)c(=O)c2ccc(C(F)(F)F)cc12] | 3.255 | >20 |
| Final Cpd X133 | 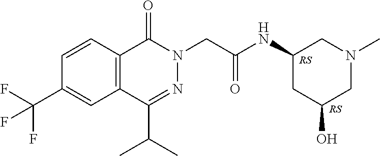 [SMILES: CC(C)c1nn(CC(=O)N[C@@H]2C[C@H](O)CN(C)C2)c(=O)c2ccc(C(F)(F)F)cc12] | 3.383 | >20 |
| Final Cpd X134 | 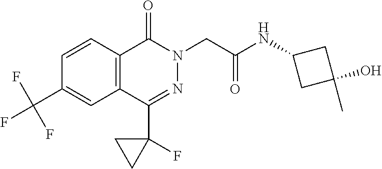 [SMILES: C[C@]1(O)C[C@@H](NC(=O)Cn2nc(C3(F)CC3)c3cc(C(F)(F)F)ccc3c2=O)C1] | 3.408 | >20 |
| Final Cpd X135 | 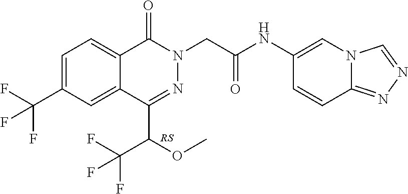 [SMILES: COC(c1nn(CC(=O)Nc2ccc3nncn3c2)c(=O)c2ccc(C(F)(F)F)cc12)C(F)(F)F] | 3.435 | >20 |
| Final Cpd X136 | 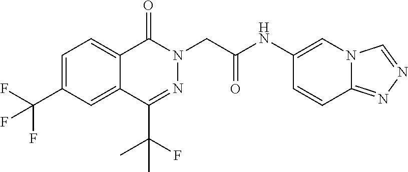 [SMILES: CC(C)(F)c1nn(CC(=O)Nc2ccc3nncn3c2)c(=O)c2ccc(C(F)(F)F)cc12] | 3.766 | >20 |
| Final Cpd X137 | 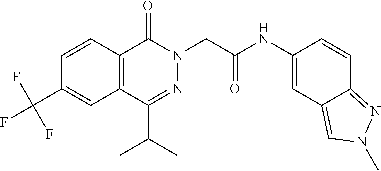 [SMILES: CC(C)c1nn(CC(=O)Nc2ccc3nn(C)cc3c2)c(=O)c2ccc(C(F)(F)F)cc12] | 3.864 | >20 |
| Final Cpd X138 | 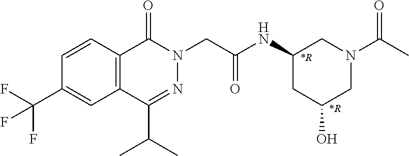 [SMILES: CC(=O)N1C[C@H](O)C[C@@H](NC(=O)Cn2nc(C(C)C)c3cc(C(F)(F)F)ccc3c2=O)C1] | 4.021 | >20 |
| Final Cpd X139 | 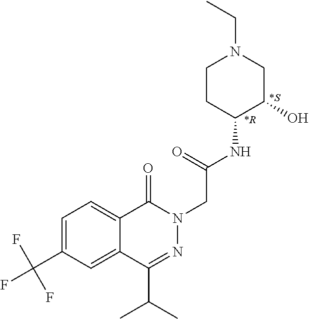 [SMILES: CCN1CC[C@@H](NC(=O)Cn2nc(C(C)C)c3cc(C(F)(F)F)ccc3c2=O)[C@@H](O)C1] | 4.276 | >20 |
| Final Cpd X140 | 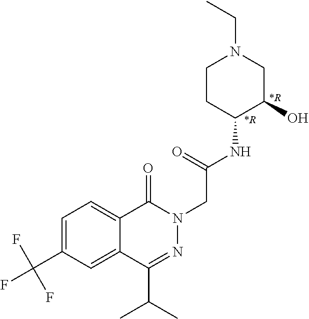 [SMILES: CCN1CC[C@@H](NC(=O)Cn2nc(C(C)C)c3cc(C(F)(F)F)ccc3c2=O)[C@H](O)C1] | 4.606 | >20 |
| Final Cpd X141 | 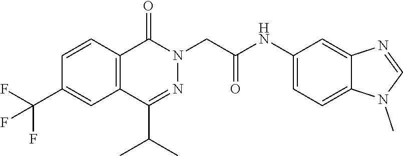 [SMILES: CC(C)c1nn(CC(=O)Nc2ccc3c(c2)ncn3C)c(=O)c2ccc(C(F)(F)F)cc12] | 4.709 | >20 |
| Final Cpd X142 | 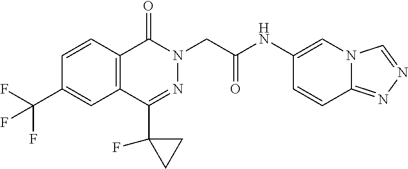 [SMILES: O=C(Cn1nc(C2(F)CC2)c2cc(C(F)(F)F)ccc2c1=O)Nc1ccc2nncn2c1] | 4.739 | >20 |
| Final Cpd X143 | 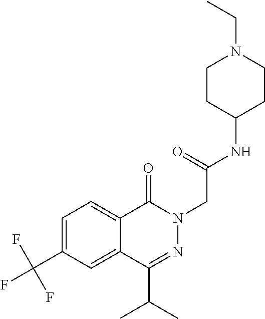 [SMILES: CCN1CCC(NC(=O)Cn2nc(C(C)C)c3cc(C(F)(F)F)ccc3c2=O)CC1] | 4.929 | >20 |
| Final Cpd X144 | 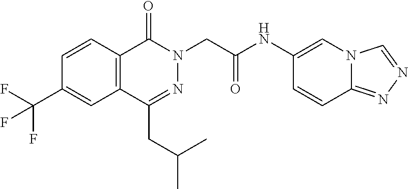 [SMILES: CC(C)Cc1nn(CC(=O)Nc2ccc3nncn3c2)c(=O)c2ccc(C(F)(F)F)cc12] | 4.945 | >20 |
| Final Cpd X145 | 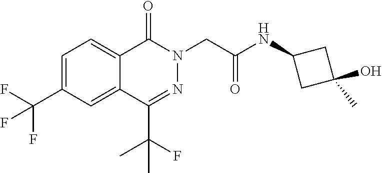 [SMILES: CC(C)(F)c1nn(CC(=O)N[C@H]2C[C@@](C)(O)C2)c(=O)c2ccc(C(F)(F)F)cc12] | 4.993 | >20 |
| Final Cpd X146 | 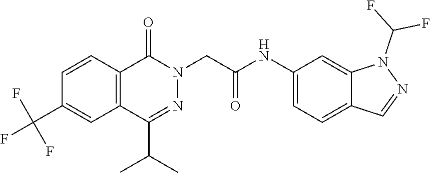 [SMILES: CC(C)c1nn(CC(=O)Nc2ccc3cnn(C(F)F)c3c2)c(=O)c2ccc(C(F)(F)F)cc12] | 5.723 | >20 |
| Final Cpd X147 | 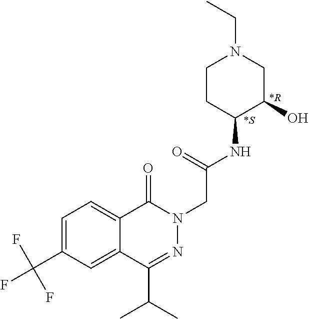 [SMILES: CCN1CC[C@H](NC(=O)Cn2nc(C(C)C)c3cc(C(F)(F)F)ccc3c2=O)[C@H](O)C1] | 5.942 | >20 |
| Final Cpd X148 | 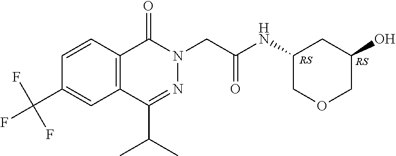 [SMILES: CC(C)c1nn(CC(=O)N[C@H]2COC[C@H](O)C2)c(=O)c2ccc(C(F)(F)F)cc12] | 6.321 | >20 |
| Final Cpd X149 | 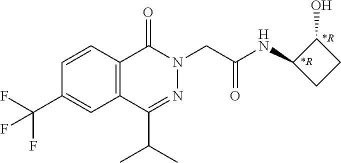 [SMILES: CC(C)c1nn(CC(=O)N[C@@H]2CC[C@H]2O)c(=O)c2ccc(C(F)(F)F)cc12] | 6.494 | >20 |
| Final Cpd X150 | 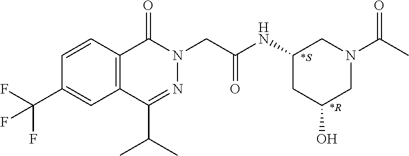 [SMILES: CC(=O)N1C[C@H](O)C[C@H](NC(=O)Cn2nc(C(C)C)c3cc(C(F)(F)F)ccc3c2=O)C1] | 7.367 | >20 |
| Final Cpd X151 | 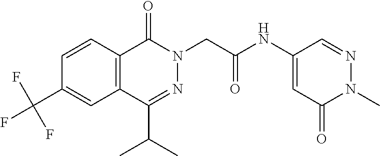 [SMILES: CC(C)c1nn(CC(=O)Nc2cnn(C)c(=O)c2)c(=O)c2ccc(C(F)(F)F)cc12] | 7.449 | >20 |
| Final Cpd X152 | 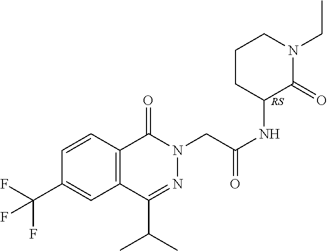 [SMILES: CCN1CCCC(NC(=O)Cn2nc(C(C)C)c3cc(C(F)(F)F)ccc3c2=O)C1=O] | 7.504 | >20 |
| Final Cpd X153 | 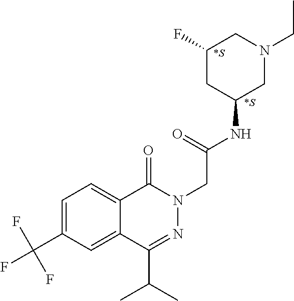 [SMILES: CCN1C[C@@H](F)C[C@H](NC(=O)Cn2nc(C(C)C)c3cc(C(F)(F)F)ccc3c2=O)C1] | 7.63 | >20 |
| Final Cpd X154 | 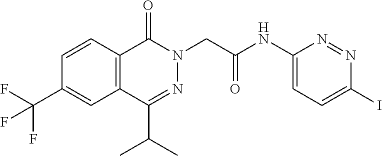 [SMILES: CC(C)c1nn(CC(=O)Nc2ccc(I)nn2)c(=O)c2ccc(C(F)(F)F)cc12] | 3.087 | >20 |
| Final Cpd X155 | 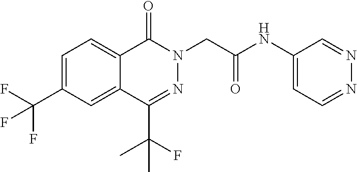 [SMILES: CC(C)(F)c1nn(CC(=O)Nc2ccnnc2)c(=O)c2ccc(C(F)(F)F)cc12] | 7.909 | 17.63 |
| Final Cpd X156 | 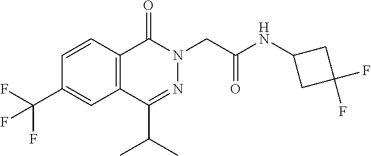 [SMILES: CC(C)c1nn(CC(=O)NC2CC(F)(F)C2)c(=O)c2ccc(C(F)(F)F)cc12] | 8.772 | >20 |
| Final Cpd X157 | 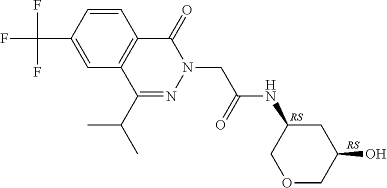 [SMILES: CC(C)c1nn(CC(=O)N[C@@H]2COC[C@H](O)C2)c(=O)c2ccc(C(F)(F)F)cc12] | 9.815 | >20 |
| Final Cpd X158 | 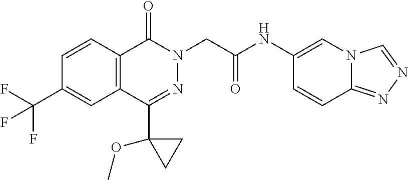 [SMILES: COC1(c2nn(CC(=O)Nc3ccc4nncn4c3)c(=O)c3ccc(C(F)(F)F)cc23)CC1] | 9.988 | >20 |
| Final Cpd X159 | 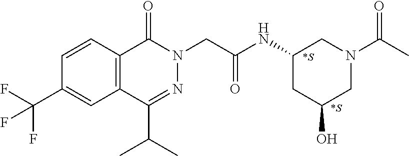 [SMILES: CC(=O)N1C[C@@H](O)C[C@H](NC(=O)Cn2nc(C(C)C)c3cc(C(F)(F)F)ccc3c2=O)C1] | 10.34 | >20 |
| Final Cpd X160 | 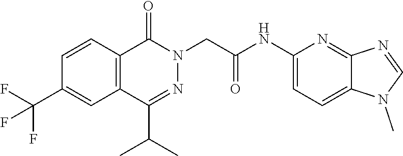 [SMILES: CC(C)c1nn(CC(=O)Nc2ccc3c(ncn3C)n2)c(=O)c2ccc(C(F)(F)F)cc12] | 13.449 | >20 |
| Final Cpd X161 | 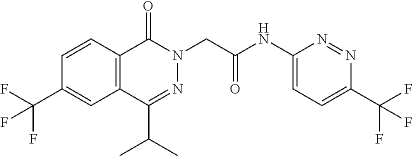 [SMILES: CC(C)c1nn(CC(=O)Nc2ccc(C(F)(F)F)nn2)c(=O)c2ccc(C(F)(F)F)cc12] | 14.213 | >20 |
| Final Cpd X162 | 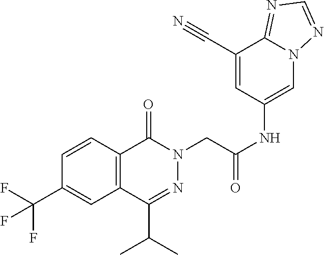 [SMILES: CC(C)c1nn(CC(=O)Nc2cc(C#N)c3ncnn3c2)c(=O)c2ccc(C(F)(F)F)cc12] | 14.98 | >20 |
| Final Cpd X163 | 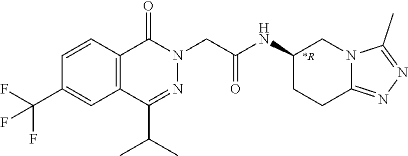 [SMILES: Cc1nnc2n1C[C@H](NC(=O)Cn1nc(C(C)C)c3cc(C(F)(F)F)ccc3c1=O)CC2] | 15.08 | >20 |
| Final Cpd X164 | 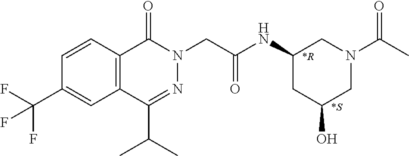 [SMILES: CC(=O)N1C[C@@H](O)C[C@@H](NC(=O)Cn2nc(C(C)C)c3cc(C(F)(F)F)ccc3c2=O)C1] | 15.329 | >20 |
| Final Cpd X165 | 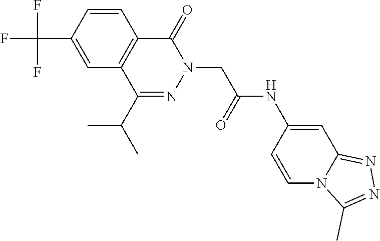 [SMILES: Cc1nnc2cc(NC(=O)Cn3nc(C(C)C)c4cc(C(F)(F)F)ccc4c3=O)ccn12] | 16.132 | >20 |
| Final Cpd X166 | 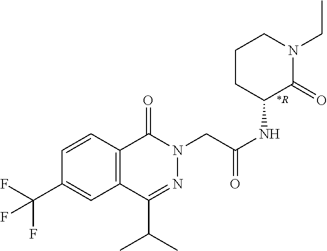 [SMILES: CCN1CCC[C@@H](NC(=O)Cn2nc(C(C)C)c3cc(C(F)(F)F)ccc3c2=O)C1=O] | 17.478 | >20 |
| Final Cpd X167 | 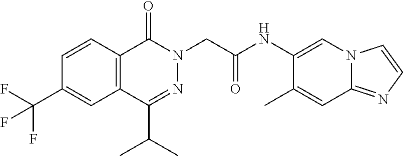 [SMILES: Cc1cc2nccn2cc1NC(=O)Cn1nc(C(C)C)c2cc(C(F)(F)F)ccc2c1=O] | 17.828 | >20 |
| Final Cpd X168 | 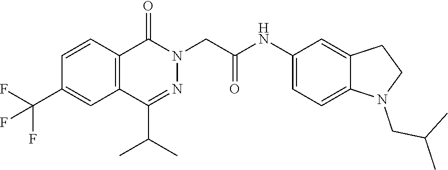 [SMILES: CC(C)CN1CCc2cc(NC(=O)Cn3nc(C(C)C)c4cc(C(F)(F)F)ccc4c3=O)ccc21] | >20 | >20 |
| Final Cpd X169 | 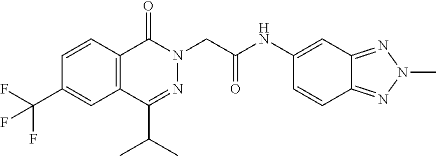 [SMILES: CC(C)c1nn(CC(=O)Nc2ccc3nn(C)nc3c2)c(=O)c2ccc(C(F)(F)F)cc12] | >20 | >20 |
| Final Cpd X170 | 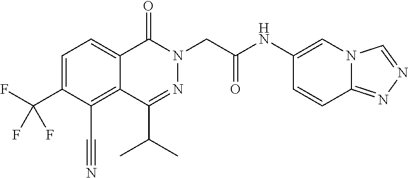 [SMILES: CC(C)c1nn(CC(=O)Nc2ccc3nncn3c2)c(=O)c2ccc(C(F)(F)F)c(C#N)c12] | >20 | >20 |
| Final Cpd X171 | 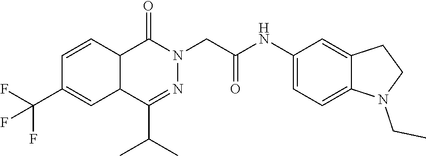 [SMILES: CCN1CCc2cc(NC(=O)CN3N=C(C(C)C)C4C=C(C(F)(F)F)C=CC4C3=O)ccc21] | >20 | >20 |
| Final Cpd X172 | 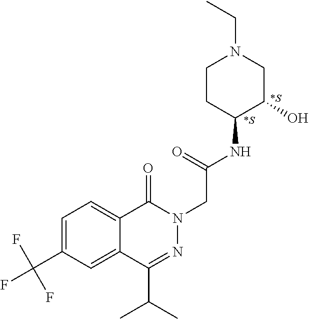 [SMILES: CCN1CC[C@H](NC(=O)Cn2nc(C(C)C)c3cc(C(F)(F)F)ccc3c2=O)[C@@H](O)C1] | >20 | >20 |
| Final Cpd X173 | 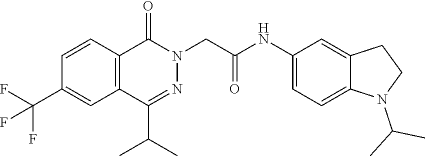 [SMILES: CC(C)c1nn(CC(=O)Nc2ccc3c(c2)CCN3C(C)C)c(=O)c2ccc(C(F)(F)F)cc12] | >20 | >20 |
| Final Cpd X174 | 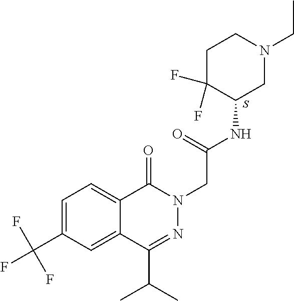 | >20 | >20 |
| Final Cpd X175 | 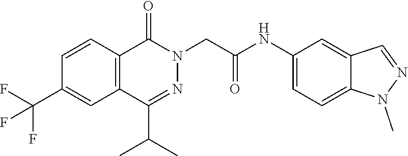 [SMILES: CC(C)c1nn(CC(=O)Nc2ccc3c(cnn3C)c2)c(=O)c2ccc(C(F)(F)F)cc12] | >20 | >20 |
| Final Cpd X176 | 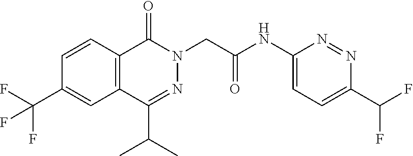 [SMILES: CC(C)c1nn(CC(=O)Nc2ccc(C(F)F)nn2)c(=O)c2ccc(C(F)(F)F)cc12] | >20 | >20 |
| Final Cpd X177 | 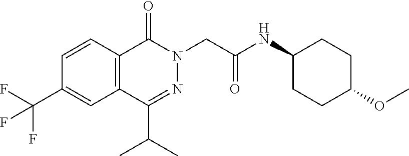 [SMILES: CO[C@H]1CC[C@H](NC(=O)Cn2nc(C(C)C)c3cc(C(F)(F)F)ccc3c2=O)CC1] | >20 | >20 |
| Final Cpd X178 | 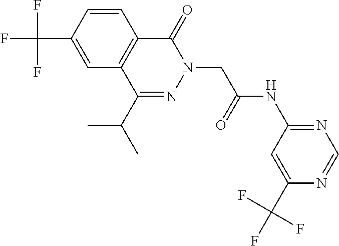 [SMILES: CC(C)c1nn(CC(=O)Nc2cc(C(F)(F)F)ncn2)c(=O)c2ccc(C(F)(F)F)cc12] | >20 | >20 |
| Final Cpd X179 | 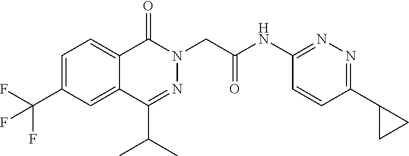 [SMILES: CC(C)c1nn(CC(=O)Nc2ccc(C3CC3)nn2)c(=O)c2ccc(C(F)(F)F)cc12] | >20 | >20 |
| Final Cpd X180 | 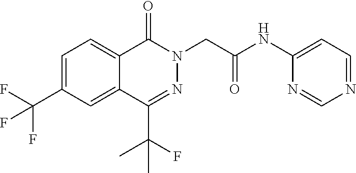 [SMILES: CC(C)(F)c1nn(CC(=O)Nc2ccncn2)c(=O)c2ccc(C(F)(F)F)cc12] | >20 | >20 |
| Final Cpd X181 | 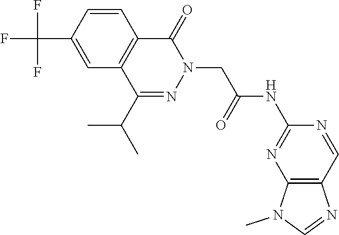 [SMILES: CC(C)c1nn(CC(=O)Nc2ncc3ncn(C)c3n2)c(=O)c2ccc(C(F)(F)F)cc12] | >20 | >20 |
| X-1013 | 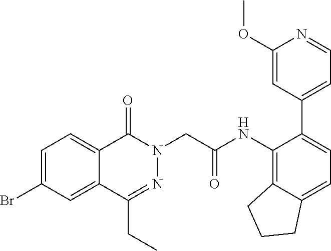 [SMILES: CCc1nn(CC(=O)Nc2c(-c3ccnc(OC)c3)ccc3c2CCC3)c(=O)c2ccc(Br)cc12] | >20 | >20 |
| X-1080 | 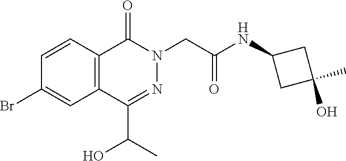 [SMILES: CC(O)c1nn(CC(=O)N[C@H]2C[C@@](C)(O)C2)c(=O)c2ccc(Br)cc12] | 0.502 | >20 |
| X-1081 | 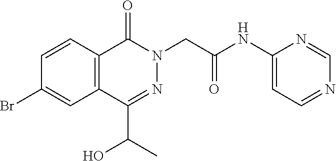 [SMILES: CC(O)c1nn(CC(=O)Nc2ccncn2)c(=O)c2ccc(Br)cc12] | 0.894 | >20 |
| X-1014 | 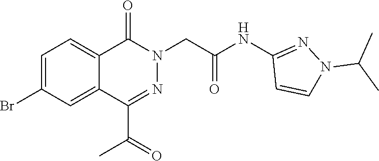 [SMILES: CC(=O)c1nn(CC(=O)Nc2ccn(C(C)C)n2)c(=O)c2ccc(Br)cc12] | >20 | >20 |
| X-1079 | 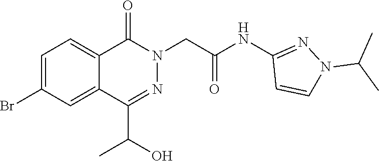 [SMILES: CC(O)c1nn(CC(=O)Nc2ccn(C(C)C)n2)c(=O)c2ccc(Br)cc12] | 9.786 | >20 |
| X-1082 | 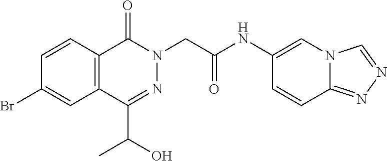 [SMILES: CC(O)c1nn(CC(=O)Nc2ccc3nncn3c2)c(=O)c2ccc(Br)cc12] | 0.346 | >20 |
| X-1018 | 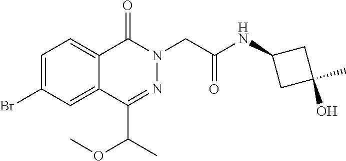 [SMILES: COC(C)c1nn(CC(=O)N[C@H]2C[C@@](C)(O)C2)c(=O)c2ccc(Br)cc12] | 0.832 | >20 |
| X-1017 | 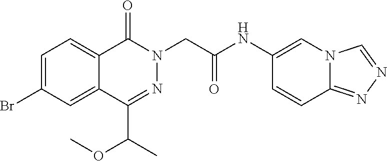 [SMILES: COC(C)c1nn(CC(=O)Nc2ccc3nncn3c2)c(=O)c2ccc(Br)cc12] | 0.506 | >20 |
| X-1016 | 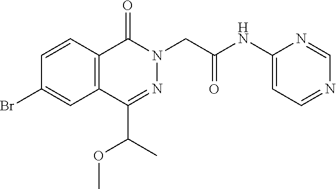 [SMILES: COC(C)c1nn(CC(=O)Nc2ccncn2)c(=O)c2ccc(Br)cc12] | 0.819 | >20 |
| X-1015 | 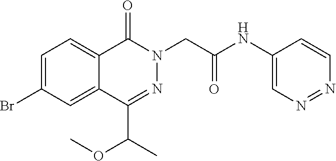 [SMILES: COC(C)c1nn(CC(=O)Nc2ccnnc2)c(=O)c2ccc(Br)cc12] | 2.131 | >20 |
| X-1019 | 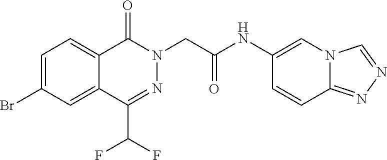 [SMILES: O=C(Cn1nc(C(F)F)c2cc(Br)ccc2c1=O)Nc1ccc2nncn2c1] | 0.183 | >20 |
| X-1000 | 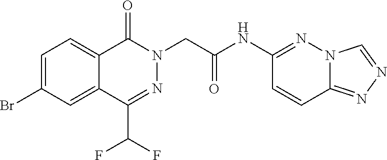 [SMILES: O=C(Cn1nc(C(F)F)c2cc(Br)ccc2c1=O)Nc1ccc2nncn2n1] | 0.122 | >20 |
| X-1020 | 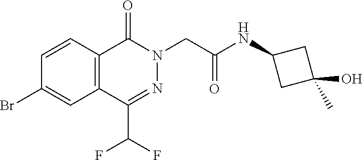 [SMILES: C[C@]1(O)C[C@@H](NC(=O)Cn2nc(C(F)F)c3cc(Br)ccc3c2=O)C1] | 0.133 | >20 |
| X-1021 | 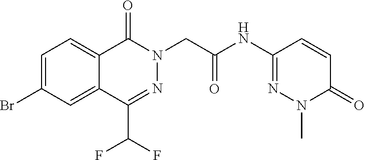 [SMILES: Cn1nc(NC(=O)Cn2nc(C(F)F)c3cc(Br)ccc3c2=O)ccc1=O] | 0.437 | >20 |
| X-1022 | 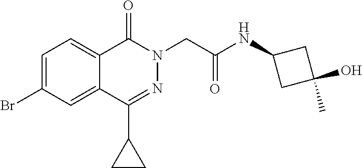 [SMILES: C[C@]1(O)C[C@@H](NC(=O)Cn2nc(C3CC3)c3cc(Br)ccc3c2=O)C1] | 0.057 | >20 |
| X-1023 | 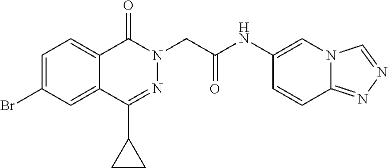 [SMILES: O=C(Cn1nc(C2CC2)c2cc(Br)ccc2c1=O)Nc1ccc2nncn2c1] | 0.101 | >20 |
| X-1001 | 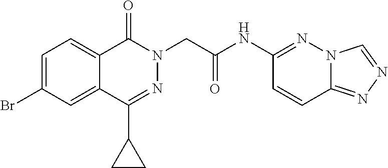 [SMILES: O=C(Cn1nc(C2CC2)c2cc(Br)ccc2c1=O)Nc1ccc2nncn2n1] | 0.224 | >20 |
| X-1024 | 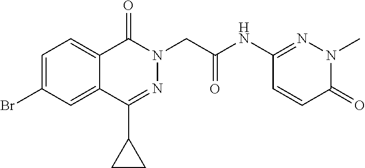 [SMILES: Cn1nc(NC(=O)Cn2nc(C3CC3)c3cc(Br)ccc3c2=O)ccc1=O] | 0.497 | >20 |
| X-1088 | 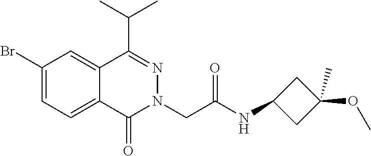 [SMILES: CO[C@]1(C)C[C@H](NC(=O)Cn2nc(C(C)C)c3cc(Br)ccc3c2=O)C1] | 0.196 | >20 |
| X-1031 | 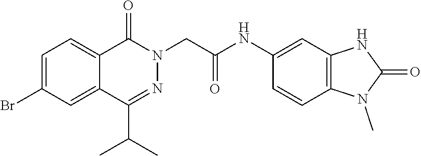 [SMILES: CC(C)c1nn(CC(=O)Nc2ccc3c(c2)[nH]c(=O)n3C)c(=O)c2ccc(Br)cc12] | 0.03 | 4.643 |
| X-1090 | 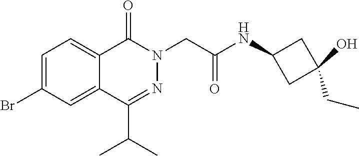 [SMILES: CC[C@]1(O)C[C@@H](NC(=O)Cn2nc(C(C)C)c3cc(Br)ccc3c2=O)C1] | 0.122 | >20 |
| X-1089 | 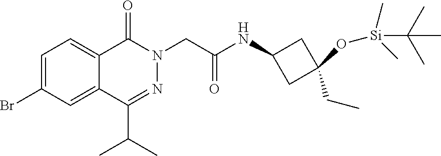 [SMILES: CC[C@]1(O[Si](C)(C)C(C)(C)C)C[C@@H](NC(=O)Cn2nc(C(C)C)c3cc(Br)ccc3c2=O)C1] | >20 | >20 |
| X-1032 | 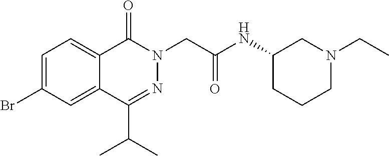 [SMILES: CCN1CCC[C@H](NC(=O)Cn2nc(C(C)C)c3cc(Br)ccc3c2=O)C1] | 0.031 | >20 |
| X-1033 | 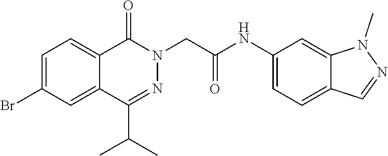 [SMILES: CC(C)c1nn(CC(=O)Nc2ccc3cnn(C)c3c2)c(=O)c2ccc(Br)cc12] | 0.848 | >20 |
| X-1004 | 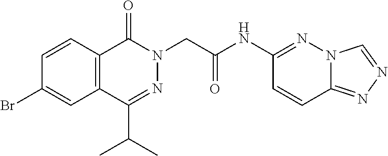 [SMILES: CC(C)c1nn(CC(=O)Nc2ccc3nncn3n2)c(=O)c2ccc(Br)cc12] | 0.041 | >20 |
| X-1035 | 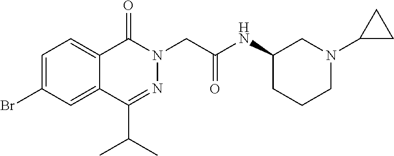 [SMILES: CC(C)c1nn(CC(=O)N[C@@H]2CCCN(C3CC3)C2)c(=O)c2ccc(Br)cc12] | 0.032 | >20 |
| X-1036 | 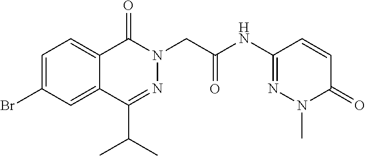 [SMILES: CC(C)c1nn(CC(=O)Nc2ccc(=O)n(C)n2)c(=O)c2ccc(Br)cc12] | 0.141 | >20 |
| X-1083 | 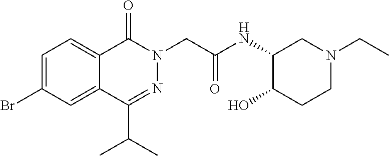 [SMILES: CCN1CC[C@H](O)[C@H](NC(=O)Cn2nc(C(C)C)c3cc(Br)ccc3c2=O)C1] | 0.018 | >20 |
| X-1005 | 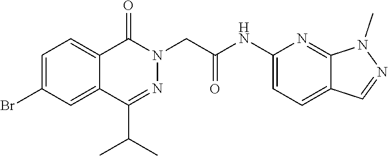 [SMILES: CC(C)c1nn(CC(=O)Nc2ccc3cnn(C)c3n2)c(=O)c2ccc(Br)cc12] | 1.47 | >20 |
| X-1034 | 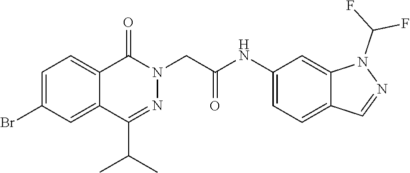 [SMILES: CC(C)c1nn(CC(=O)Nc2ccc3cnn(C(F)F)c3c2)c(=O)c2ccc(Br)cc12] | 3.01 | >20 |
| X-1063 | 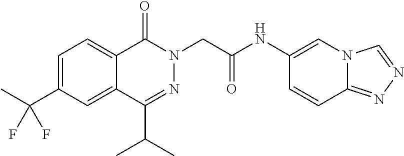 [SMILES: CC(C)c1nn(CC(=O)Nc2ccc3nncn3c2)c(=O)c2ccc(C(C)(F)F)cc12] | 0.016 | 1.85 |
| X-1003 | 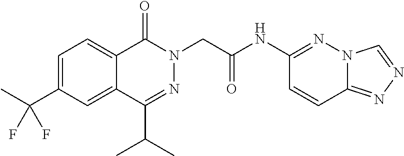 [SMILES: CC(C)c1nn(CC(=O)Nc2ccc3nncn3n2)c(=O)c2ccc(C(C)(F)F)cc12] | 0.04 | 9.895 |
| X-1062 | 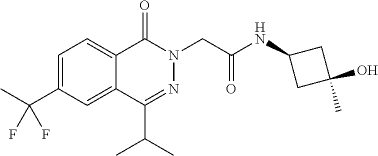 [SMILES: CC(C)c1nn(CC(=O)N[C@H]2C[C@@](C)(O)C2)c(=O)c2ccc(C(C)(F)F)cc12] | 0.09 | >20 |
| X-1006 | 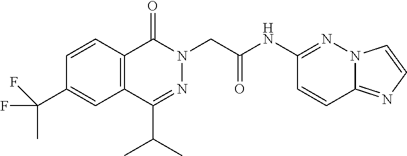 [SMILES: CC(C)c1nn(CC(=O)Nc2ccc3nccn3n2)c(=O)c2ccc(C(C)(F)F)cc12] | 0.128 | 4.47 |
| X-1085 | 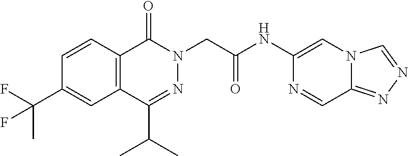 [SMILES: CC(C)c1nn(CC(=O)Nc2cn3cnnc3cn2)c(=O)c2ccc(C(C)(F)F)cc12] | 0.143 | >20 |
| X-1066 | 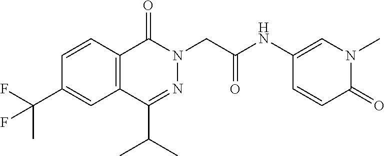 [SMILES: CC(C)c1nn(CC(=O)Nc2ccc(=O)n(C)c2)c(=O)c2ccc(C(C)(F)F)cc12] | 0.156 | 6.783 |
| X-1067 | 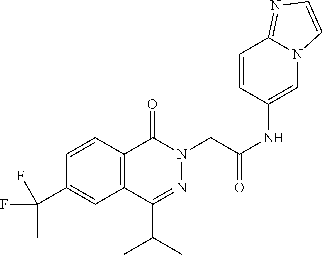 [SMILES: CC(C)c1nn(CC(=O)Nc2ccc3nccn3c2)c(=O)c2ccc(C(C)(F)F)cc12] | 0.175 | 13.195 |
| X-1065 | 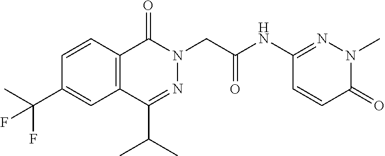 [SMILES: CC(C)c1nn(CC(=O)Nc2ccc(=O)n(C)n2)c(=O)c2ccc(C(C)(F)F)cc12] | 0.293 | 15.689 |
| X-1064 | 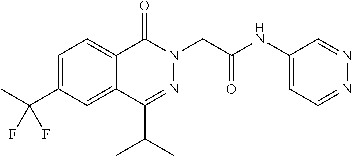 [SMILES: CC(C)c1nn(CC(=O)Nc2ccnnc2)c(=O)c2ccc(C(C)(F)F)cc12] | 0.533 | 18.932 |
| X-1052 | 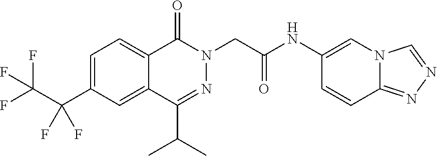 [SMILES: CC(C)c1nn(CC(=O)Nc2ccc3nncn3c2)c(=O)c2ccc(C(F)(F)C(F)(F)F)cc12] | 1.262 | >20 |
| X-1053 | 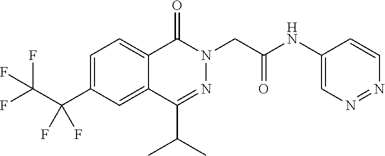 [SMILES: CC(C)c1nn(CC(=O)Nc2ccnnc2)c(=O)c2ccc(C(F)(F)C(F)(F)F)cc12] | 3.183 | >20 |
| X-1060 | 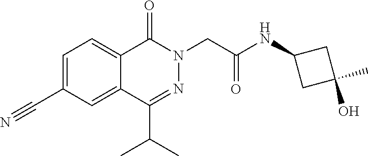 [SMILES: CC(C)c1nn(CC(=O)N[C@H]2C[C@@](C)(O)C2)c(=O)c2ccc(C#N)cc12] | 1.741 | >20 |
| X-1061 | 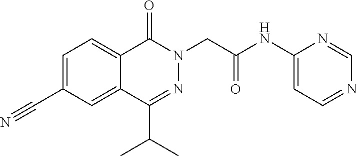 [SMILES: CC(C)c1nn(CC(=O)Nc2ccncn2)c(=O)c2ccc(C#N)cc12] | >20 | >20 |
| X-1029 | 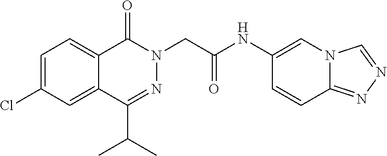 [SMILES: CC(C)c1nn(CC(=O)Nc2ccc3nncn3c2)c(=O)c2ccc(Cl)cc12] | 0.041 | >20 |
| X-1027 | 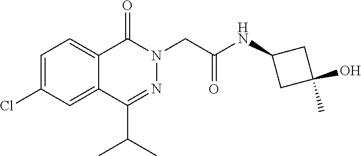 [SMILES: CC(C)c1nn(CC(=O)N[C@H]2C[C@@](C)(O)C2)c(=O)c2ccc(Cl)cc12] | 0.06 | >20 |
| X-1028 | 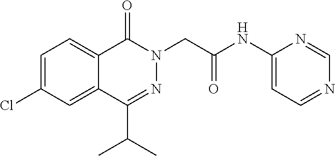 [SMILES: CC(C)c1nn(CC(=O)Nc2ccncn2)c(=O)c2ccc(Cl)cc12] | 0.128 | >20 |
| X-1071 | 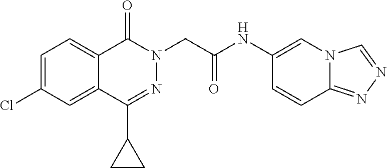 [SMILES: O=C(Cn1nc(C2CC2)c2cc(Cl)ccc2c1=O)Nc1ccc2nncn2c1] | 0.398 | >20 |
| X-1070 | 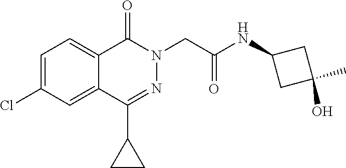 [SMILES: C[C@]1(O)C[C@@H](NC(=O)Cn2nc(C3CC3)c3cc(Cl)ccc3c2=O)C1] | 0.406 | >20 |
| X-1030 | 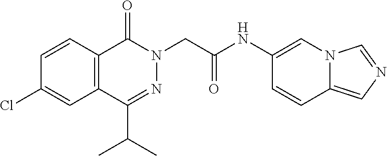 [SMILES: CC(C)c1nn(CC(=O)Nc2ccc3cncn3c2)c(=O)c2ccc(Cl)cc12] | 0.436 | >20 |
| X-1002 | 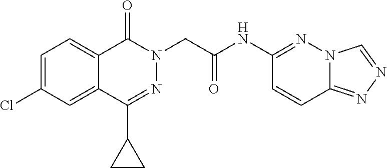 [SMILES: O=C(Cn1nc(C2CC2)c2cc(Cl)ccc2c1=O)Nc1ccc2nncn2n1] | 0.453 | >20 |
| X-1072 | 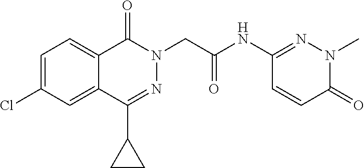 [SMILES: Cn1nc(NC(=O)Cn2nc(C3CC3)c3cc(Cl)ccc3c2=O)ccc1=O] | 1.4 | >20 |
| X-1046 | 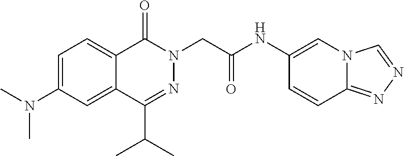 [SMILES: CC(C)c1nn(CC(=O)Nc2ccc3nncn3c2)c(=O)c2ccc(N(C)C)cc12] | 0.422 | >20 |
| X-1047 | 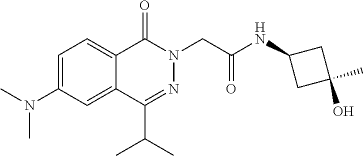 | 1.566 | >20 |
| X-1037 | 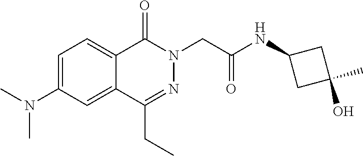 | 2.335 | >20 |
| X-1040 | 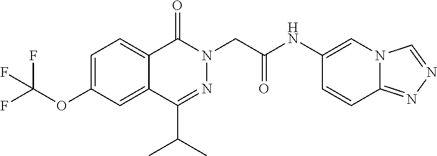 [SMILES: CC(C)c1nn(CC(=O)Nc2ccc3nncn3c2)c(=O)c2ccc(OC(F)(F)F)cc12] | 1.133 | 7.193 |
| X-1041 | 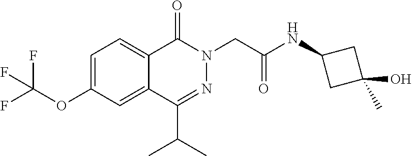 [SMILES: CC(C)c1nn(CC(=O)N[C@H]2C[C@@](C)(O)C2)c(=O)c2ccc(OC(F)(F)F)cc12] | 1.334 | >20 |
| X-1038 | 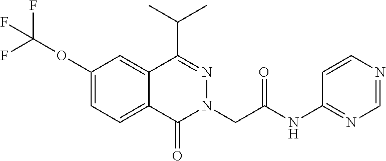 [SMILES: CC(C)c1nn(CC(=O)Nc2ccncn2)c(=O)c2ccc(OC(F)(F)F)cc12] | 2.511 | 9.546 |
| X-1025 | 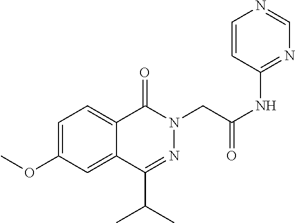 [SMILES: COc1ccc2c(=O)n(CC(=O)Nc3ccncn3)nc(C(C)C)c2c1] | 2.515 | >20 |
| X-1039 | 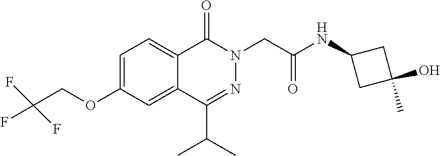 [SMILES: CC(C)c1nn(CC(=O)N[C@H]2C[C@@](C)(O)C2)c(=O)c2ccc(OCC(F)(F)F)cc12] | 13.029 | >20 |
| X-1054 | 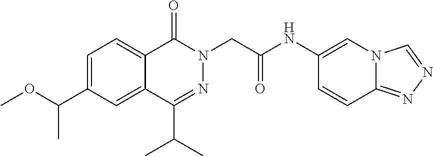 [SMILES: COC(C)c1ccc2c(=O)n(CC(=O)Nc3ccc4nncn4c3)nc(C(C)C)c2c1] | 0.539 | 18.561 |
| X-1056 | 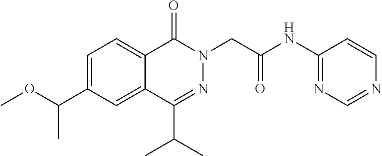 [SMILES: COC(C)c1ccc2c(=O)n(CC(=O)Nc3ccncn3)nc(C(C)C)c2c1] | 1.609 | 14.002 |
| X-1057 | 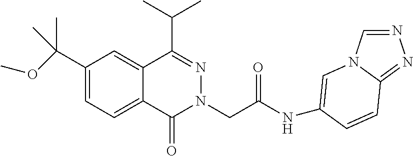 [SMILES: COC(C)(C)c1ccc2c(=O)n(CC(=O)Nc3ccc4nncn4c3)nc(C(C)C)c2c1] | 1.912 | 17.454 |
| X-1055 | 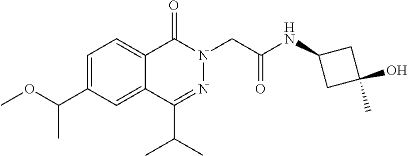 [SMILES: COC(C)c1ccc2c(=O)n(CC(=O)N[C@H]3C[C@@](C)(O)C3)nc(C(C)C)c2c1] | 2.133 | >20 |
| X-1059 | 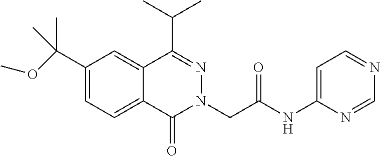 [SMILES: COC(C)(C)c1ccc2c(=O)n(CC(=O)Nc3ccncn3)nc(C(C)C)c2c1] | 2.424 | >20 |
| X-1058 | 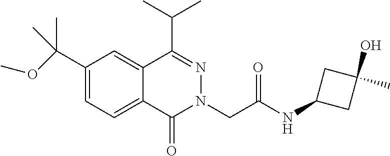 | 4.555 | >20 |
| X-1042 | 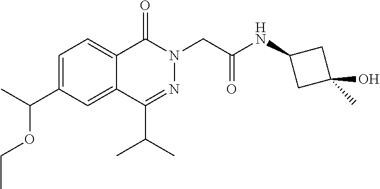 [SMILES: CCOC(C)c1ccc2c(=O)n(CC(=O)N[C@H]3C[C@@](C)(O)C3)nc(C(C)C)c2c1] | 12.204 | >20 |
| X-1007 | 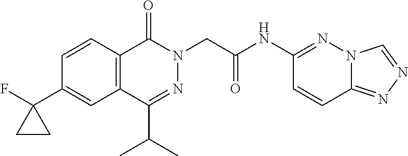 [SMILES: CC(C)c1nn(CC(=O)Nc2ccc3nncn3n2)c(=O)c2ccc(C3(F)CC3)cc12] | 0.015 | 2.133 |
| X-1074 | 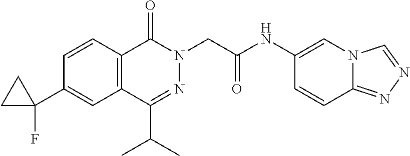 [SMILES: CC(C)c1nn(CC(=O)Nc2ccc3nncn3c2)c(=O)c2ccc(C3(F)CC3)cc12] | 0.027 | 14.781 |
| X-1008 | 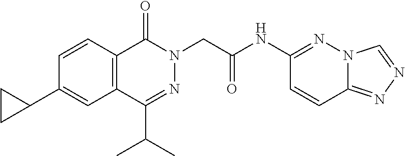 [SMILES: CC(C)c1nn(CC(=O)Nc2ccc3nncn3n2)c(=O)c2ccc(C3CC3)cc12] | 0.033 | 2.606 |
| X-1075 | 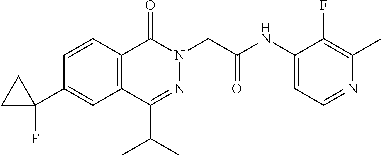 [SMILES: Cc1nccc(NC(=O)Cn2nc(C(C)C)c3cc(C4(F)CC4)ccc3c2=O)c1F] | 0.153 | >20 |
| X-1069 | 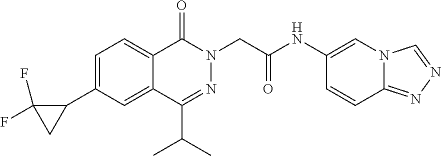 [SMILES: CC(C)c1nn(CC(=O)Nc2ccc3nncn3c2)c(=O)c2ccc(C3CC3(F)F)cc12] | 1.136 | 14.109 |
| X-1068 | 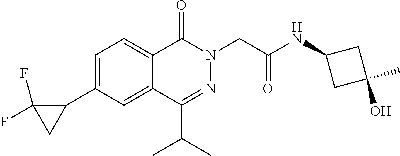 [SMILES: CC(C)c1nn(CC(=O)N[C@H]2C[C@@](C)(O)C2)c(=O)c2ccc(C3CC3(F)F)cc12] | 2.796 | >20 |
| X-1026 | 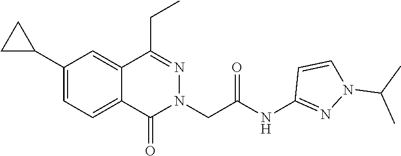 [SMILES: CCc1nn(CC(=O)Nc2ccn(C(C)C)n2)c(=O)c2ccc(C3CC3)cc12] | >20 | >20 |
| X-1073 | 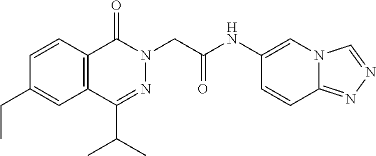 [SMILES: CCc1ccc2c(=O)n(CC(=O)Nc3ccc4nncn4c3)nc(C(C)C)c2c1] | 0.22 | >20 |
| X-1010 | 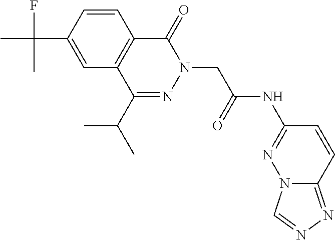 [SMILES: CC(C)c1nn(CC(=O)Nc2ccc3nncn3n2)c(=O)c2ccc(C(C)(C)F)cc12] | 0.017 | 3.113 |
| X-1076 | 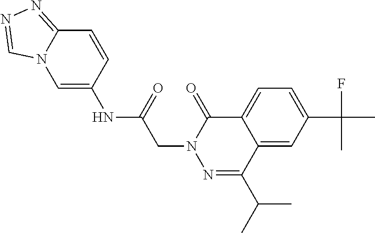 [SMILES: CC(C)c1nn(CC(=O)Nc2ccc3nncn3c2)c(=O)c2ccc(C(C)(C)F)cc12] | 0.033 | 19.692 |
| X-1009 | 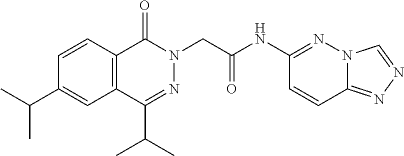 [SMILES: CC(C)c1ccc2c(=O)n(CC(=O)Nc3ccc4nncn4n3)nc(C(C)C)c2c1] | 0.045 | 2.829 |
| X-1011 | 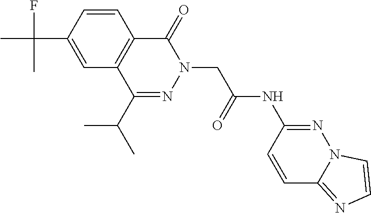 [SMILES: CC(C)c1nn(CC(=O)Nc2ccc3nccn3n2)c(=O)c2ccc(C(C)(C)F)cc12] | 0.089 | 7.561 |
| X-1051 | 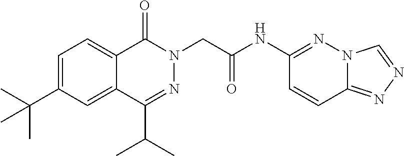 [SMILES: CC(C)c1nn(CC(=O)Nc2ccc3nncn3n2)c(=O)c2ccc(C(C)(C)C)cc12] | 0.106 | 7.019 |
| X-1086 | 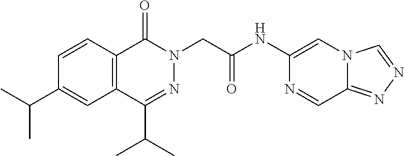 [SMILES: CC(C)c1ccc2c(=O)n(CC(=O)Nc3cn4cnnc4cn3)nc(C(C)C)c2c1] | 0.137 | >20 |
| X-1077 | 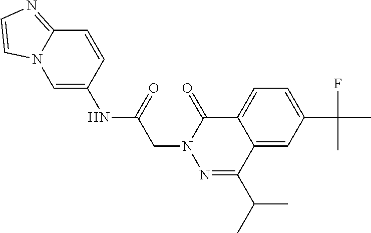 [SMILES: CC(C)c1nn(CC(=O)Nc2ccc3nccn3c2)c(=O)c2ccc(C(C)(C)F)cc12] | 0.144 | 16.527 |
| X-1048 | 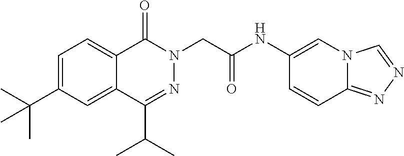 [SMILES: CC(C)c1nn(CC(=O)Nc2ccc3nncn3c2)c(=O)c2ccc(C(C)(C)C)cc12] | 0.27 | 8.084 |
| X-1049 | 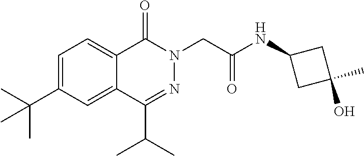 [SMILES: CC(C)c1nn(CC(=O)N[C@H]2C[C@@](C)(O)C2)c(=O)c2ccc(C(C)(C)C)cc12] | 0.426 | >20 |
| X-1050 | 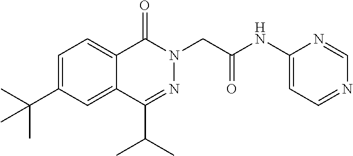 [SMILES: CC(C)c1nn(CC(=O)Nc2ccncn2)c(=O)c2ccc(C(C)(C)C)cc12] | 0.99 | >20 |
| X-1045 | 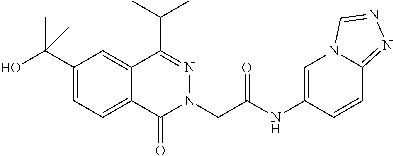 [SMILES: CC(C)c1nn(CC(=O)Nc2ccc3nncn3c2)c(=O)c2ccc(C(C)(C)O)cc12] | 3.758 | >20 |
| X-1044 | 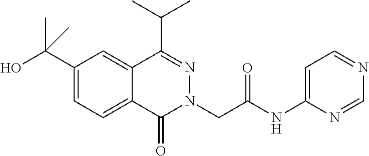 [SMILES: CC(C)c1nn(CC(=O)Nc2ccncn2)c(=O)c2ccc(C(C)(C)O)cc12] | 4.38 | 8.291 |
| X-1043 | 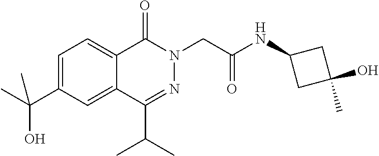 [SMILES: CC(C)c1nn(CC(=O)N[C@H]2C[C@@](C)(O)C2)c(=O)c2ccc(C(C)(C)O)cc12] | 10.975 | >20 |
| X-1012 | 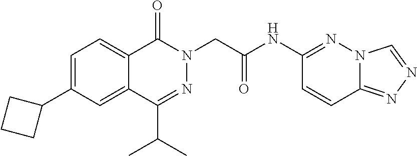 [SMILES: CC(C)c1nn(CC(=O)Nc2ccc3nncn3n2)c(=O)c2ccc(C3CCC3)cc12] | 0.18 | 1.083 |
| X-1078 | 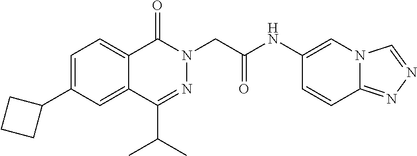 [SMILES: CC(C)c1nn(CC(=O)Nc2ccc3nncn3c2)c(=O)c2ccc(C3CCC3)cc12] | 1.309 | >20 |

### Example D—Further Testing

One or more compound(s) of the invention (including compounds of the final examples) is/are tested in a number of other methods to evaluate, amongst other properties, permeability, stability (including metabolic stability and blood stability) and solubility.

### Permeability Test

The in vitro passive permeability and the ability to be a transported substrate of P-glycoprotein (P-gp) is tested using MDCKcells stably transduced with MDR1 (this may be performed at a commercial organisation offering ADME, PK services, e.g. Cyprotex). Permeability experiments are conducted in duplicate at a single concentration (5 μM) in a transwell system with an incubation of 120 min. The apical to basolateral (AtoB) transport in the presence and absence of the P-gp inhibitor GF120918 and the basolateral to apical (BtoA) transport in the absence of the P-gp inhibitor is measured and permeation rates (Apparent Permeability) of the test compounds (Pₐₚₚ×10⁻⁶ cm/sec) are calculated.

### Metabolic Stability Test in Liver Microsomes

The metabolic stability of a test compound is tested (this may be performed at a commercial organisation offering ADME, PK services, e.g. Cyprotex) by using liver microsomes (0.5 mg/ml protein) from human and preclinical species incubated up to 60 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (ml/min/mg microsomal protein) is calculated using the following formula:

$${Cl}_{int} = \frac{0.693}{{t}_{1 / 2}} \times \frac{{V}_{i n c}}{{W}_{mic prot , inc}}$$

Where: V_{inc}=incubation volume,
W_{mic prot,inc}=weight of microsomal protein in the incubation.

### Metabolic Stability Test in Liver Hepatocytes

The metabolic stability of a test compound is tested using liver hepatocytes (1 milj cells) from human and preclinical species incubated up to 120 minutes at 37° C. with 1 μM test compound.

The in vitro metabolic half-life (t_{1/2}) is calculated using the slope of the log-linear regression from the percentage parent compound remaining versus time relationship (κ),

t_{1/2}=−ln(2)/κ.

The in vitro intrinsic clearance (Clᵢₙₜ) (μl/min/million cells) is calculated using the following formula:

$${Cl}_{int} = \frac{0.693}{{t}_{1 / 2}} \times \frac{{V}_{i n c}}{\# {cells}_{inc}} \times 1000$$

Where: V_{inc}=incubation volume,
#cells_{inc}=number of cells (×10⁶) in the incubation

### Solubility Test

The test/assay is run in triplicate and is semi-automated using the Tecan Fluent for all liquid handling with the following general steps:
20 μl of 10 mM stock solution is dispensed in a 500p 96 well plate
DMSO is evaporated (Genevac)
a stir bar and 400 μl of buffer/biorelevant media is added
the solution is stirred for 72 h (pH2 and pH7) or 24 h (FaSSIF and FeSSIF)
the solution is filtered
the filtrate is quantified by UPLC/UV using a three-points calibration curve
   The LC conditions are:
Waters Acquity UPLC
Mobile phase A: 0.1% formic acid in H2O, B: 0.1% formic acid in CH3CN
Column: Waters HSS T3 1.8 μm 2.1×50 mm
Column temp.: 55° C.
Inj.vol.: 2 μl
Flow: 0.6 ml/min
Wavelength UV: 250_350 nm
Gradient: 0 min: 0% B, 0.3 min: 5% B, 1.8 min: 95% B, 2.6 min: 95% B

### BLOOD STABILITY ASSAY

The compound of the invention/examples is spiked at a certain concentration in plasma or blood from the agreed preclinical species; then after incubating to predetermined times and conditions (37° C., 0° C. (ice) or room temperature) the concentration of the test compound in the blood or plasma matrix with LCMS/MS can then be determined.

1. A compound of formula (I), 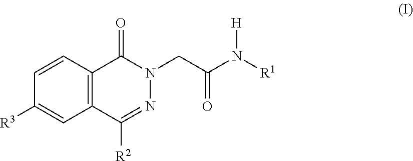
or a pharmaceutically acceptable salt thereof, wherein:
R¹ represents:
(i) C₃₋₆ cycloalkyl optionally substituted with one or more substituents independently selected from —OH and —C₁₋₃ alkyl;
(ii) aryl or heteroaryl, each of which is optionally substituted with 1 to 3 substituents independently selected from halo, —OH, —O—C₁₋₃ alkyl, —C₁₋₃ alkyl, haloC₁₋₃alkyl, hydroxyC₁₋₃ alkyl, C₁₋₃ alkoxy, haloC₁₋₃alkoxy; or
(iii) heterocyclyl, optionally substituted with 1 to 3 substituents independently selected from C₁₋₃ alkyl and C₃₋₆ cycloalkyl;
R² represents:
(i) C₁₋₃ alkyl optionally substituted with one or more substituents independently selected from halo, —OH and —OC₁₋₃ alkyl;
(ii) C₃₋₆ cycloalkyl;
(iii) C₂₋₄ alkenyl optionally substituted with —OC₁₋₃ alkyl; or
(iv) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
(i) hydrogen;
(ii) halo;
(iii) C₁₋₄ alkyl optionally substituted with one or more substituents independently selected from halo, —OH and —OC₁₋₃ alkyl;
(iv) C₂₋₄ alkenyl optionally substituted with —OC₁₋₃ alkyl;
(v) C₃₋₆ cycloalkyl; or
(vi) —OC₁₋₃ alkyl,
provided that:
(i) when R³ represents hydrogen, R² represents methyl, then R¹ does not represent 4-methylphenyl;
(ii) when R³ represents hydrogen, R² represents cyclohexyl, then R¹ does not represent 2-indanyl (2,3-dihydro-1H-indene linked at the 2-position).

2. The compound of claim 1, wherein:
R³ represents:
(i) halo;
(ii) C₁₋₄ alkyl optionally substituted with one or more substituents independently selected from halo, —OH and —OC₁₋₃ alkyl;
(iii) C₂₋₄ alkenyl optionally substituted with —OC₁₋₃ alkyl;
(iv) C₃₋₆ cycloalkyl; or
(v) —OC₁₋₃ alkyl.

3. The compound of claim 1, wherein R¹ represents C₃₋₆ cycloalkyl optionally substituted by one or two substituents selected from C₁₋₃ alkyl and —OH.

4. The compound of claim 3, wherein R¹ represents: 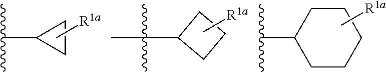
where each R^{1a} represents one or two optional substituents selected from —OH and C₁₋₃ alkyl.

5. The compound of claim 1, wherein R¹ represents: (i) phenyl; (ii) a 6-membered mono-cyclic heteroaryl group; or (iii) a 9- or 10-membered bicyclic heteroaryl group, all of which are optionally substituted with one or two substituent(s) selected from halo, —OH, C₁₋₃ alkyl and —OC₁₋₃ alkyl.

6. The compound of claim 5, wherein R¹ represents phenyl or a mono-cyclic 6-membered heteroaryl group: 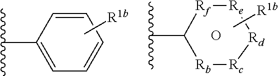
wherein R^{1b} represents one or two optional substituents selected from halo, —CH₃, —OH and —OCH₃, and, either one or two of R_{b}, R_{c}, R_{d}, Rₑ and R_{f} represent(s) a nitrogen heteroatom (and the others represent a CH).

7. The compound of claim 5, wherein R¹ represents a 9- or 10-membered bicyclic heteroaryl group, for instance: 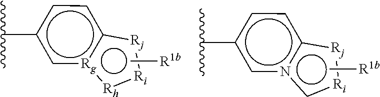
wherein R^{1b} represents one or two optional substituent selected from halo, —OH and —OCH₃, each ring of the bicyclic system is aromatic, R_{g} represents a N or C atom and any one or two of Rₕ, Rᵢ and Rⱼ represents N and the other(s) represent(s) C.

8. The compound of claim 1, wherein R² represents: (i) C₁₋₃ alkyl optionally substituted with one or more substituents independently selected from halo, —OH and —OC₁₋₂ alkyl; (ii) C₃₋₆ cycloalkyl; or (iii) C₂₋₄ alkenyl optionally substituted with —OC₁₋₂ alkyl.

9. The compound of claim 8, wherein R² represents unsubstituted C₁₋₃ alkyl.

10. The compound of claim 1, wherein R³ represents (i) hydrogen; (ii) halo; (iii) C₁₋₄ alkyl optionally substituted with one or more substituents independently selected from halo, —OH and —OC₁₋₂ alkyl; (iv) C₃₋₆ cycloalkyl; or (v) —OC₁₋₃ alkyl.

11. The compound of claim 1, wherein R³ represents (i) hydrogen; (ii) bromo; (iii) C₁₋₃ alkyl optionally substituted by one or more fluoro atoms; (iv) cyclopropyl; or (v) —OC₁₋₂ alkyl.

12. A compound of formula (I) as defined in claim 1, or a pharmaceutically acceptable salt thereof, wherein:
R¹ represents:
(i) C₃₋₆ cycloalkyl optionally substituted with one or more substituents independently selected from halo, —OH, —C₁₋₃ alkyl (itself optionally substituted by one or more substituents selected from fluoro and —OH) and —OC₁₋₃alkyl;
(ii) aryl or heteroaryl, each of which is optionally substituted with 1 to 3 substituents independently selected from halo, —CN, —OH, —O—C₁₋₃ alkyl, —C₁₋₆ alkyl (e.g. —C₁₋₃ alkyl), haloC₁₋₃alkyl, hydroxyC₁₋₃ alkyl, C₁₋₃ alkoxyC₁₋₃alkyl, haloC₁₋₃alkoxy, aminoC₁₋₃alkyl (e.g. H₂N—C₁₋₃alkyl or (CH₃)₂N—C₁₋₃ alkyl), C₃₋₆ cycloalkyl or aryl/heteroaryl (wherein such latter groups are themselves optionally substituted by one or more substituents selected from halo, C₁₋₃ alkyl and —OC₁₋₃ alkyl); or
(iii) heterocyclyl, optionally substituted with 1 to 3 substituents independently selected from halo, ═O, —OH, —C₁₋₄ alkyl (itself optionally substituted by one or more substituents selected from fluoro, ═O and —OH), —OC₁₋₃alkyl, C₃₋₆ cycloalkyl and a 3-6 membered heterocyclyl ring;
R² represents:
(i) C₁₋₆ alkyl (e.g C₁₋₄ alkyl or C₁₋₃ alkyl) optionally substituted with one or more substituents independently selected from halo, ═O, —OH and —OC₁₋₃ alkyl;
(ii) C₃₋₆ cycloalkyl optionally substituted by one or more substituents selected from halo (e.g. fluoro), C₁₋₃ alkyl and —OC₁₋₃ alkyl;
(iii) C₂₋₄ alkenyl optionally substituted with —OC₁₋₃ alkyl; or
(iv) —N(R^{2a})R^{2b};
R^{2a} and R^{2b} each represent hydrogen or C₁₋₄ alkyl, or R^{2a} and R^{2b} may be linked together to form a 3- to 4-membered ring optionally substituted by one or more fluoro atoms;
R³ represents:
(i) hydrogen;
(ii) halo or —CN;
(iii) C₁₋₆ alkyl (e.g. C₁₋₄ alkyl) optionally substituted with one or more substituents independently selected from halo, —OH and —OC₁₋₃ alkyl;
(iv) C₂₋₄ alkenyl optionally substituted with —OC₁₋₃ alkyl;
(v) C₃₋₆ cycloalkyl optionally substituted by one or more fluoro atoms;
(vi) —NH2, —N(H)(C₁₋₃alkyl) or N(C₁₋₃alkyl)₂; or
(vii) —OC₁₋₃ alkyl optionally substituted by one or more fluoro atoms;
and wherein the R³ containing benzene ring may also be optionally substituted (at the three relevant positions) with one substituent selected from halo (e.g. fluoro), —OH and —CN.

13. A compound as claimed in claim 12, wherein:
R³ represents:
(i) halo or —CN;
(ii) C₁₋₆ alkyl (e.g. C₁₋₄ alkyl) optionally substituted with one or more substituents independently selected from halo, —OH and —OC₁₋₃ alkyl;
(iii) C₂₋₄ alkenyl optionally substituted with —OC₁₋₃ alkyl;
(iv) C₃₋₆ cycloalkyl optionally substituted by one or more fluoro atoms;
(v) —NH₂, —N(H)(C₁₋₃alkyl) or N(C₁₋₃alkyl)₂; or
(vi) —OC₁₋₃ alkyl optionally substituted by one or more fluoro atoms.

14. A pharmaceutical composition comprising a therapeutically effective amount of a compound as defined in claim 1 and a pharmaceutically acceptable carrier.

15. (canceled)

16. (canceled)

17. A combination comprising: (a) a compound according to claim 1; and (b) one or more other therapeutic agents.

18. (canceled)

19. A method of treating a disease or disorder associated with inhibition of NLRP3 inflammasome activity in a subject in need thereof, the method comprising administering to said subject a therapeutically effective amount of a compound according to claim 1.

20. The method of treating according to claim 19 wherein the disease or disorder associated with inhibition of NLRP3 inflammasome activity is selected from inflammasome related diseases and disorders, immune diseases, inflammatory diseases, auto-immune diseases, auto-inflammatory fever syndromes, cryopyrin-associated periodic syndrome, chronic liver disease, viral hepatitis, non-alcoholic steatohepatitis, alcoholic steatohepatitis, alcoholic liver disease, inflammatory arthritis related disorders, gout, chondrocalcinosis, osteoarthritis, rheumatoid arthritis, chronic arthropathy, acute arthropathy, kidney related disease, hyperoxaluria, lupus nephritis, Type I and Type II diabetes, nephropathy, retinopathy, hypertensive nephropathy, hemodialysis related inflammation, neuroinflammation-related diseases, multiple sclerosis, brain infection, acute injury, neurodegenerative diseases, Alzheimer's disease, cardiovascular diseases, metabolic diseases, cardiovascular risk reduction, hypertension, atherosclerosis, peripheral artery disease, acute heart failure, inflammatory skin diseases, acne, wound healing and scar formation, asthma, sarcoidosis, age-related macular degeneration, colon cancer, lung cancer, myeloproliferative neoplasms, leukemias, myelodysplastic syndromes and myelofibrosis.

21. A process for the preparation of a compound of formula (I) as claimed in claim 1, which comprises:
(i) reaction of a compound of formula (II), 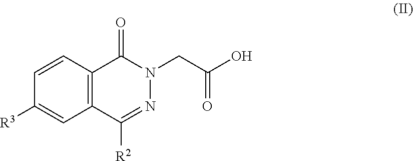
or a derivative thereof, wherein R² and R³ are as defined in claim 1, with a compound of formula (III),
H₂N—R¹ (III)
or a derivative thereof, wherein R¹ is as defined in claim 1, under amide-forming reaction conditions;
(ii) reaction of a compound of formula (IV), 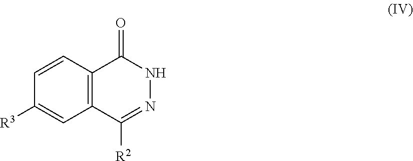
wherein R² and R³ are as defined in claim 1, with a compound of formula (V),
LG^{a}-CH₂—C(O)—N(H)R¹ (V)
wherein LG^{a} represents a suitable leaving group and R¹ is as defined in claim 1;
(iii) by transformation of a certain compound of formula (I) into another.

22. A compound of formula (II) or a compound of formula (IV): 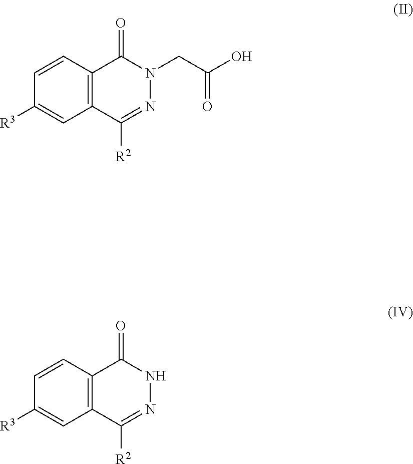
wherein R² and R³ are as defined in claim 1.
